(19) United States
(12) Patent Application Publication
(10) Pub. No.: US 2024/0309349 A1
(43) Pub. Date: Sep. 19, 2024

(54) COMPOSITIONS AND METHODS FOR NUCLEIC ACID MODIFICATIONS

(71) Applicant: ACRIGEN BIOSCIENCES, Berkeley, CA (US)

(72) Inventors: David Rabuka, Berkeley, CA (US); Allison Sharrar, Berkeley, CA (US); Michael Schelle, Berkeley, CA (US); Luisa Mayumi Arake De Tacca, Berkeley, CA (US); Mia Christina Pulos-Holmes, Berkeley, CA (US)

(21) Appl. No.: 18/577,942

(22) PCT Filed: Jul. 8, 2022
(86) PCT No.: PCT/US22/73556

§ 371 Date: Jan. 9, 2024

Related U.S. Application Data
(60) Provisional application No. 63/220,008, filed on Jul. 9, 2021.
(60) Provisional application No. 63/343,810, filed on May 19, 2022.

(51) Int. Cl. C12N 9/22; A61K 48/00; C07K 16/28; C12N 5/0783; C12N 15/11; C12N 15/82; C12N 15/90

(52) U.S. Cl. CPC C12N 9/22; A61K 48/005; C07K 16/2803; C12N 5/0636; C12N 15/11; C12N 15/8213; C12N 15/907; C07K 2317/53; C07K 2319/03; C07K 2319/09; C12N 2310/20; C12N 2510/00; C12N 2800/80

(57) ABSTRACT

The present disclosure provides nucleases and compositions, methods, and systems thereof for nucleic acid modification. More particularly, the present disclosure provides compositions and system comprising a nuclease comprising an amino acid sequence having at least 70% identity, at least 80% identity, at least 90% identity, at least 95% identity or at least 99% identity to any of SEQ ID NOs: 1-23 or 32-35 and at least one gRNA.

### CROSS-REFERENCE TO RELATED APPLICATIONS

This application claims the benefit of U.S. Provisional Application Nos. 63/220,008, filed Jul. 9, 2021, and 63/343,810, filed May 19, 2022, the contents of which are herein incorporated by reference in their entirety.

### FIELD

The present invention relates to nucleases and compositions, methods, and systems thereof for nucleic acid modification.

### SEQUENCE LISTING STATEMENT

The contents of the electronic sequence listing titled (ACRIG-39644.601.xml; Size: 720,155 bytes; and Date of Creation: Jul. 7, 2022) is herein incorporated by reference in its entirety.

### BACKGROUND

Clustered regularly interspaced short palindromic repeats (CRISPR)-associated (Cas) nucleases dominate the nucleic acid-editing landscape because they are versatile, rapid, and easy-to-use editing tools. The most well-characterized CRISPR-Cas nuclease, Cas9, utilizes one or more RNAs to act as a sequence-specific targeting element linking the nuclease to the target nucleic acid. However, presently CRISPR/Cas systems have some limitations for use in eukaryotic organisms including: inefficient delivery to mature cells in large numbers, low efficiency of editing, off-target events, target sequence preferences, and optimal temperatures and conditions for enzymatic activity.

### SUMMARY

Provided herein are engineered nucleases having the amino acid sequence of at least 70% identity, at least 80% identity, at least 85% identity, at least 90% identity, or at least 95% identity to an amino acid sequence of SEQ ID NOs: 1-23 or 32-35. Provided herein are engineered nucleases having the amino acid sequence of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to an amino acid sequence of SEQ ID NOs: 1-23 or 32-35. In some embodiments, the engineered nuclease comprises an amino acid sequence having at least 90% identity (e.g., at least 90%, at least 91%, at least 92%, at least 93%, at least 94%, at least 95%, at least 96%, at least 97%, at least 98%, at least 99%) to an amino acid sequence of SEQ ID NOs: 1-23 or 32-35. In select embodiments, the engineered nuclease comprises an amino acid sequence selected from the group consisting of SEQ ID NOs: 1-23, 26-29 or 32-236. In some embodiments, the engineered nuclease comprises an amino acid sequence with less than 50% sequence identity with SEQ ID NO: 24.

Provided herein are engineered nucleases comprising an amino acid sequence with at least 90% or at least 95% identity to SEQ ID NO: 8 with one or more amino acid replacements corresponding to positions selected from the group consisting of 174, 283, 604, and 598. In some embodiments, the one or more amino acid replacements is selected from the group consisting of E174R, N283A, S598R, and K604R. In some embodiments, the amino acid sequence lacks one or more of the following features: E at position 174, N at position 283, K at position 604 and S at position 598. In some embodiments, the engineered nuclease comprises two amino acid replacements at positions selected from the group consisting of 174, 283, 604, and 598. In some embodiments, the engineered nuclease comprises three amino acid replacements at positions selected from the group consisting of 174, 283, 604, and 598. In some embodiments, the engineered nuclease comprises one or more of SEQ ID NOs: 419-421.

Provided herein are engineered nucleases comprising an amino acid sequence with at least 90% or at least 95% identity to SEQ ID NO: 1 with one or more amino acid replacements corresponding to positions selected from the group consisting of 139, 231, 491, 496, 502 and 779. In some embodiments, the one or more amino acid replacements is selected from the group consisting of S139R, N231A, N491R, G496R, K502R and A779L. In some embodiments, the amino acid sequence Jacks one or more of the following features: S at position 139, N at position 231, N at position 491, G at position 496, K at position 502 and A at position 779. In some embodiments, the engineered nuclease comprises two amino acid replacements at positions selected from the group consisting of 139, 231, 491, 496, 502 and 779. In some embodiments, the engineered nuclease comprises three amino acid replacements at positions selected from the group consisting of 139, 231, 491, 496, 502 and 779. In some embodiments, the engineered nuclease comprises one or more of SEQ ID NOs: 422-424.

Provided herein are engineered nucleases comprising an amino acid sequence with at least 90% or at least 95% identity to SEQ ID NO: 2 with one or more amino acid replacements corresponding to positions selected from the group consisting of 139, 231, 496, 491, and 502. In some embodiments, the one or more amino acid replacements is selected from the group consisting of S139R, N231A, N491R, D496R, and Y502R. In some embodiments, the amino acid sequence lacks one or more of the following features: S at position 139, N at position 231, N at position 491, D at position 496, and Y at position 502. In some embodiments, the engineered nuclease comprises two amino acid replacements at positions selected from the group consisting of 139, 231, 491, 496, and 502. In some embodiments, the engineered nuclease comprises three amino acid replacements at positions selected from the group consisting of 139, 231, 491, 496, and 502. In some embodiments, the engineered nuclease comprises one or more of SEQ ID NOs: 425-427.

Provided herein are engineered nucleases comprising an amino acid sequence with at least 90% or at least 95% identity to SEQ ID NO: 3 with one or more amino acid replacements corresponding to positions selected from the group consisting of 175, 272, 544, 549, and 555. In some embodiments, the one or more amino acid replacements is selected from the group consisting of E175R, N272A, N544R, T549R, and K555R. In some embodiments, the amino acid sequence lacks one or more of the following features: E at position 175, N at position 272, N at position 544, T at position 549, and K at position 555. In some embodiments, the engineered nuclease comprises two amino acid replacements at positions selected from the group consisting of 175, 272, 544, 549, and 555. In some embodiments, the engineered nuclease comprises three amino acid replacements at positions selected from the group consisting of 175, 272, 544, 549, and 555. In some embodiments, the engineered nuclease comprises one or more of SEQ ID NOs: 428-430.

Provided herein are engineered nucleases comprising an amino acid sequence with at least 90% or at least 95% identity to SEQ ID NO: 4 with one or more amino acid replacements corresponding to positions selected from the group consisting of 179, 261, 544, 549, and 555. In some embodiments, the one or more amino acid replacements is selected from the group consisting of K179R, N261A, Y544R, G549R, and K555R. In some embodiments, the amino acid sequence lacks one or more of the following features: K at position 179, N at position 261, Y at position 544, G at position 549, and K at position 555. In some embodiments, the engineered nuclease comprises two amino acid replacements at positions selected from the group consisting of 179, 261, 544, 549, and 555. In some embodiments, the engineered nuclease comprises three amino acid replacements at positions selected from the group consisting of 179, 261, 544, 549, and 555. In some embodiments, the engineered nuclease comprises one or more of SEQ ID NOs: 431-433.

Provided herein are engineered nucleases comprising an amino acid sequence with at least 90% or at least 95% identity to SEQ ID NO: 5 with one or more amino acid replacements corresponding to positions selected from the group consisting of 161, 261, 534, 539, 545, and 803. In some embodiments, the one or more amino acid replacements is selected from the group consisting of T161R, N261A, C534R, N539R, K545R and Q803L. In some embodiments, the amino acid sequence lacks one or more of the following features: T at position 161, N at position 261, C at position 534, N at position 539, K at position 545, G at position 549, and Q at position 803. In some embodiments, the engineered nuclease comprises two amino acid replacements at positions selected from the group consisting of 161, 261, 534, 539, 545, and 803. In some embodiments, the engineered nuclease comprises three amino acid replacements at positions selected from the group consisting of 161, 261, 534, 539, 545, and 803. In some embodiments, the engineered nuclease comprises one or more of SEQ ID NOs: 434-436.

Provided herein are engineered nucleases comprising an amino acid sequence with at least 90% or at least 95% identity to SEQ ID NO: 6 with one or more amino acid replacements corresponding to positions selected from the group consisting of 137, 242, 523, 528, and 534. In some embodiments, the one or more amino acid replacements is selected from the group consisting of T137R, N242A, N523R, N528R, and K534R. In some embodiments, the amino acid sequence lacks one or more of the following features: T at position 137, N at position 242, N at position 523, N at position 528, and K at position 534. In some embodiments, the engineered nuclease comprises two amino acid replacements at positions selected from the group consisting of 137, 242, 523, 528, and 534. In some embodiments, the engineered nuclease comprises three amino acid replacements at positions selected from the group consisting of 137, 242, 523, 528, and 534. In some embodiments, the engineered nuclease comprises one or more of SEQ ID NOs: 437-439.

Provided herein are engineered nucleases comprising an amino acid sequence with at least 90% or at least 95% identity to SEQ ID NO: 7 with one or more amino acid replacements corresponding to positions selected from the group consisting of 160, 265, 542, 547, 553, and 842. In some embodiments, the one or more amino acid replacements is selected from the group consisting of E160R, N265A, N542R, D547R, K553R, and E842L. In some embodiments, the amino acid sequence lacks one or more of the following features: E at position 160, N at position 265, N at position 542, D at position 547, K at position 553, and E at position 842. In some embodiments, the engineered nuclease comprises two amino acid replacements at positions selected from the group consisting of 160, 265, 542, 547, 553, and 842. In some embodiments, the engineered nuclease comprises three amino acid replacements at positions selected from the group consisting of 160, 265, 542, 547, 553, and 842. In some embodiments, the engineered nuclease comprises one or more of SEQ ID NOs: 440-442.

Provided herein are engineered nucleases comprising an amino acid sequence with at least 90% or at least 95% identity to SEQ ID NO: 9 with one or more amino acid replacements corresponding to positions selected from the group consisting of 161, 244, 516, 521, and 527. In some embodiments, the one or more amino acid replacements is selected from the group consisting of E161R, N244A, S516R, G521R, and K527R. In some embodiments, the amino acid sequence lacks one or more of the following features: E at position 161, N at position 244, S at position 516, G at position 521, and K at position 527. In some embodiments, the engineered nuclease comprises two amino acid replacements at positions selected from the group consisting of 161, 244, 516, 521, and 527. In some embodiments, the engineered nuclease comprises three amino acid replacements at positions selected from the group consisting of 161, 244, 516, 521, and 527. In some embodiments, the engineered nuclease comprises one or more of SEQ ID NOs: 443-445.

Provided herein are engineered nucleases comprising an amino acid sequence with at least 90% or at least 95% identity to SEQ ID NO. 10 with one or more amino acid replacements corresponding to positions selected from the group consisting of 151, 255, 523, 528, and 534. In some embodiments, the one or more amino acid replacements is selected from the group consisting of N151R, N255A, N523R, G528R, and K534R. In some embodiments, the amino acid sequence lacks one or more of the following features: N at position 151, N at position 255, N at position 523, G at position 528, and K at position 534. In some embodiments, the engineered nuclease comprises two amino acid replacements at positions selected from the group consisting of 151, 255, 523, 528, and 534. In some embodiments, the engineered nuclease comprises three amino acid replacements at positions selected from the group consisting of 151, 255, 523, 528, and 534. In some embodiments, the engineered nuclease comprises one or more of SEQ ID NOs: 446-448.

Provided herein are engineered nucleases comprising an amino acid sequence with at least 90% or at least 95% identity to SEQ ID NO: 11 with one or more amino acid replacements corresponding to positions selected from the group consisting of 187, 267, 547, 552, and 558. In some embodiments, the one or more amino acid replacements is selected from the group consisting of Q187R, N267A, K547R, M552R, and E558R. In some embodiments, the amino acid sequence lacks one or more of the following features: Q at position 187, N at position 267, K at position 547, M at position 552, and E at position 558. In some embodiments, the engineered nuclease comprises two amino acid replacements at positions selected from the group consisting of 187, 267, 547, 552, and 558. In some embodiments, the engineered nuclease comprises three amino acid replacements at positions selected from the group consisting of 187, 267, 547, 552, and 558. In some embodiments, the engineered nuclease comprises one or more of SEQ ID NOs: 449-451.

Provided herein are engineered nucleases comprising an amino acid sequence with at least 90% or at least 95% identity to SEQ ID NO: 12 with one or more amino acid replacements corresponding to positions selected from the group consisting of 158, 263, 531, 526, and 537 In some embodiments, the one or more amino acid replacements is selected from the group consisting of K158R, N263A, N531R, C526R, and K537R. In some embodiments, the amino acid sequence lacks one or more of the following features: K at position 158, N at position 263, N at position 531, C at position 526, and K at position 537. In some embodiments, the engineered nuclease comprises two amino acid replacements at positions selected from the group consisting of 158, 263, 531, 526, and 537. In some embodiments, the engineered nuclease comprises three amino acid replacements at positions selected from the group consisting of 158, 263, 531, 526, and 537. In some embodiments, the engineered nuclease comprises one or more of SEQ ID NOs: 452-454.

Provided herein are engineered nucleases comprising an amino acid sequence with at least 90% or at least 95% identity to SEQ ID NO: 13 with one or more amino acid replacements corresponding to positions selected from the group consisting of 158, 263, 531, 526, and 537. In some embodiments, the one or more amino acid replacements is selected from the group consisting of K158R, N263A, N531R, C526R, and K537R. In some embodiments, the amino acid sequence lacks one or more of the following features: K at position 158, N at position 263, N at position 531, C at position 526, and K at position 537. In some embodiments, the engineered nuclease comprises two amino acid replacements at positions selected from the group consisting of 158, 263, 531, 526, and 537. In some embodiments, the engineered nuclease comprises three amino acid replacements at positions selected from the group consisting of 158, 263, 531, 526, and 537. In some embodiments, the engineered nuclease comprises one or more of SEQ ID NOs: 455-457.

Provided herein are engineered nucleases comprising an amino acid sequence with at least 90% or at least 95% identity to SEQ ID NO: 14 with one or more amino acid replacements corresponding to positions selected from the group consisting of 159, 259, 532, 537, 543 and 801. In some embodiments, the one or more amino acid replacements is selected from the group consisting of K159R, N259A, N532R, N537R, K543R and Q801L. In some embodiments, the amino acid sequence lacks one or more of the following features: K at position 159, N at position 259, N at position 532, N at position 537, K at position 543, and Q at position 801. In some embodiments, the engineered nuclease comprises two amino acid replacements at positions selected from the group consisting of 159, 259, 532, 537, 543 and 801. In some embodiments, the engineered nuclease comprises three amino acid replacements at positions selected from the group consisting of 159, 259, 532, 537, 543 and 801. In some embodiments, the engineered nuclease comprises one or more of SEQ ID NOs: 458-460.

Provided herein are engineered nucleases comprising an amino acid sequence with at least 90% or at least 95% identity to SEQ ID NO. 15 with one or more amino acid replacements corresponding to positions selected from the group consisting of 158, 264, 525, 530, and 536. In some embodiments, the one or more amino acid replacements is selected from the group consisting of E158R, N264A, N525R, N530R, and K536R. In some embodiments, the amino acid sequence lacks one or more of the following features. E at position 158, N at position 264, N at position 525, N at position 530, and K at position 536. In some embodiments, the engineered nuclease comprises two amino acid replacements at positions selected from the group consisting of 158, 264, 525, 530, and 536. In some embodiments, the engineered nuclease comprises three amino acid replacements at positions selected from the group consisting of 158, 264, 525, 530, and 536. In some embodiments, the engineered nuclease comprises one or more of SEQ ID NOs: 461-463.

Provided herein are engineered nucleases comprising an amino acid sequence with at least 90% or at least 95% identity to SEQ ID NO: 16 with one or more amino acid replacements corresponding to positions selected from the group consisting of 175, 256, 542, 548, and 537. In some embodiments, the one or more amino acid replacements is selected from the group consisting of K175R, N256A, C537R, T542R and K548R. In some embodiments, the amino acid sequence lacks one or more of the following features: K at position 175, N at position 256, C at position 537, T at position 542, and K at position 548. In some embodiments, the engineered nuclease comprises two amino acid replacements at positions selected from the group consisting of 175, 256, 542, 548, and 537. In some embodiments, the engineered nuclease comprises three amino acid replacements at positions selected from the group consisting of 175, 256, 542, 548, and 537. In some embodiments, the engineered nuclease comprises one or more of SEQ ID NOs: 464-466.

Provided herein are engineered nucleases comprising an amino acid sequence with at least 90% or at least 95% identity to SEQ ID NO: 17 with one or more amino acid replacements corresponding to positions selected from the group consisting of 160, 265, 527, 532, and 538. In some embodiments, the one or more amino acid replacements is selected from the group consisting of K160R, N265A, C527R, S532R, and K538R. In some embodiments, the amino acid sequence lacks one or more of the following features. K at position 160, N at position 265, C at position 527, S at position 532, and K at position 538. In some embodiments, the engineered nuclease comprises two amino acid replacements at positions selected from the group consisting of 160, 265, 527, 532, and 538. In some embodiments, the engineered nuclease comprises three amino acid replacements at positions selected from the group consisting of 160, 265, 527, 532, and 538. In some embodiments, the engineered nuclease comprises one or more of SEQ ID NOs: 467-469.

Provided herein are engineered nucleases comprising an amino acid sequence with at least 90% or at least 95% identity to SEQ ID NO: 18 with one or more amino acid replacements corresponding to positions selected from the group consisting of 152, 256, 515, 520, 526, and 775. In some embodiments, the one or more amino acid replacements is selected from the group consisting of E152R, N256A, N515R, DS20R, K526R and Q775L. In some embodiments, the amino acid sequence lacks one or more of the following features: E at position 152, N at position 256, N at position 515, D at position 520, K at position 526, and Q at position 775. In some embodiments, the engineered nuclease comprises two amino acid replacements at positions selected from the group consisting of 152, 256, 515, 520, 526, and 775. In some embodiments, the engineered nuclease comprises three amino acid replacements at positions selected from the group consisting of 152, 256, 515, 520, 526, and 775. In some embodiments, the engineered nuclease comprises one or more of SEQ ID NOs: 470-472.

Provided herein are engineered nucleases comprising an amino acid sequence with at least 90% or at least 95% identity to SEQ ID NO: 19 with one or more amino acid replacements corresponding to positions selected from the group consisting of 154, 259, 531, 536, 542, and 802. In some embodiments, the one or more amino acid replacements is selected from the group consisting of T154R, N259A, N531R, G536R, K542R and S802L. In some embodiments, the amino acid sequence lacks one or more of the following features: T at position 154, N at position 259, N at position 531, G at position 536, K at position 542, and S at position 802. In some embodiments, the engineered nuclease comprises two amino acid replacements at positions selected from the group consisting of 154, 259, 531, 536, 542, and 802. In some embodiments, the engineered nuclease comprises three amino acid replacements at positions selected from the group consisting of 154, 259, 531, 536, 542, and 802. In some embodiments, the engineered nuclease comprises one or more of SEQ ID NOs: 473-475.

Provided herein are engineered nucleases comprising an amino acid sequence with at least 90% or at least 95% identity to SEQ ID NO: 20 with one or more amino acid replacements corresponding to positions selected from the group consisting of 155, 261, 540, 545, and 551. In some embodiments, the one or more amino acid replacements is selected from the group consisting of E155R, N261A, N540R, G545R, and K551R. In some embodiments, the amino acid sequence lacks one or more of the following features: E at position 155, N at position 261, N at position 540, G at position 545, and K at position 551. In some embodiments, the engineered nuclease comprises two amino acid replacements at positions selected from the group consisting of 155, 261, 540, 545, and 551. In some embodiments, the engineered nuclease comprises three amino acid replacements at positions selected from the group consisting of 155, 261, 540, 545, and 551. In some embodiments, the engineered nuclease comprises one or more of SEQ ID NOs: 476-478.

Provided herein are engineered nucleases comprising an amino acid sequence with at least 90% or at least 95% identity to SEQ ID NO: 21 with one or more amino acid replacements corresponding to positions selected from the group consisting of 155, 264, 542, 547, and 553. In some embodiments, the one or more amino acid replacements is selected from the group consisting of K155R, N264A, N542R, D547R and K553R. In some embodiments, the amino acid sequence lacks one or more of the following features: K at position 155, N at position 264, N at position 542, D at position 547, and K at position 553. In some embodiments, the engineered nuclease comprises two amino acid replacements at positions selected from the group consisting of 155, 264, 542, 547, and 553. In some embodiments, the engineered nuclease comprises three amino acid replacements at positions selected from the group consisting of 155, 264, 542, 547, and 553. In some embodiments, the engineered nuclease comprises one or more of SEQ ID NOs: 479-481.

Provided herein are engineered nucleases comprising an amino acid sequence with at least 90% or at least 95% identity to SEQ ID NO: 22 with one or more amino acid replacements corresponding to positions selected from the group consisting of 175, 280, 543, 548, and 554. In some embodiments, the one or more amino acid replacements is selected from the group consisting of K175R, N280A, C543R, N548R and K554R. In some embodiments, the amino acid sequence lacks one or more of the following features: K at position 175, N at position 280, C at position 543, N at position 548, and K at position 554. In some embodiments, the engineered nuclease comprises two amino acid replacements at positions selected from the group consisting of 175, 280, 543, 548, and 554. In some embodiments, the engineered nuclease comprises three amino acid replacements at positions selected from the group consisting of 175, 280, 543, 548, and 554. In some embodiments, the engineered nuclease comprises one or more of SEQ ID NOs: 482-484.

Provided herein are engineered nucleases comprising an amino acid sequence with at least 90% or at least 95% identity to SEQ ID NO. 23 with one or more amino acid replacements corresponding to positions selected from the group consisting of 171, 277, 558, 563, 569, and 827. In some embodiments, the one or more amino acid replacements is selected from the group consisting of E171R, N277A, N558R, DS63R, K569R and Q827L. In some embodiments, the amino acid sequence lacks one or more of the following features: E at position 171, N at position 277, N at position 558, N at position 563, K at position 569 and Q at position 827. In some embodiments, the engineered nuclease comprises two amino acid replacements at positions selected from the group consisting of 171, 277, 558, 563, 569, and 827. In some embodiments, the engineered nuclease comprises three amino acid replacements at positions selected from the group consisting of 171, 277, 558, 563, 569, and 827. In some embodiments, the engineered nuclease comprises one or more of SEQ ID NOs: 485-487.

In some embodiments, the engineered nuclease does not contain an amino acid sequence having SEQ ID NO: 488.

In some embodiments, the engineered nucleases disclosed herein further comprise a nuclear localization sequence (NLS). In some embodiments, the NLS sequence is on the C-terminal end of the nuclease.

Nucleic acid molecules comprising a sequence encoding an engineered nuclease, as disclosed herein, are also provided. In some embodiments, the nucleic acid molecule comprises a mRNA or a vector.

Compositions and systems comprising the engineered nucleases disclosed herein are additionally provided. Also provided are compositions and systems comprising a nuclease comprising an amino acid sequence having at least 70% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) identity to any of SEQ ID NOs: 1-23 or 32-35 or a nucleic acid molecule comprising a sequence encoding the nuclease. In some embodiments, the nuclease comprises an amino acid sequence having at least 90% (e.g., 90%, 91%, 92%, 93%, 94%, 95%, 96%, 97%, 98%, 99%, or 100%) identity to any of SEQ ID NOs. 1-23 or 32-35. In some embodiments, the amino acid sequence has less than 50% sequence identity with SEQ ID NO: 24. In some embodiments, the nuclease comprises an amino acid sequence selected from the group consisting of SEQ ID NOs: 1-23, 26-29, and 32-236. In some embodiments, the nuclease further comprises a nuclear localization sequence (NLS). In some embodiments, the nucleic acid molecule comprising a sequence encoding the nuclease comprises a messenger RNA or a vector.

In some embodiments, the compositions or systems further comprise at least one guide RNA (gRNA) or a nucleic acid comprising a sequence encoding the gRNA.

In some embodiments, the at least one gRNA comprises a non-naturally occurring gRNA. In some embodiments, the at least one gRNA is encoded in a CRISPR RNA array.

In some embodiments, the nucleic acid molecule encoding each one or both of the nuclease or engineered nuclease and the gRNA comprises a messenger RNA, a vector, or a combination thereof. In some embodiments, the nuclease or engineered nuclease and the gRNA are encoded on the same nucleic acid. In some embodiments, the nuclease or engineered nuclease and the gRNA are encoded on different nucleic acids.

In some embodiments, the system further comprises a target nucleic acid.

In some embodiments, the system is a cell-free system.

Also provided are cells comprising the disclosed compositions and systems. In some embodiments, the cell is a prokaryotic cell. In some embodiments, the cell is a eukaryotic cell (e.g., a mammalian cell or a human cell).

Further provided are methods for modifying a target nucleic acid comprising contacting the target nucleic acid with a nuclease, composition or system as described herein.

In some embodiments, the target nucleic acid sequence is in a cell. In some embodiments, the cell is a prokaryotic cell. In some embodiments, the cell is a eukaryotic cell (e.g., a mammalian cell or a human cell).

In some embodiments, introducing the system or composition into the cell comprises administering the system or composition to a subject. In some embodiments, administering comprises in vivo administration. In some embodiments, administering comprises transplantation of ex vivo treated cells comprising the system or composition.

Additionally provided are methods of generating a cell that expresses a recombinant receptor. In some embodiments, the methods comprise introducing into the cell: an engineered nuclease, as disclosed herein, or a nucleic acid molecule comprising a sequence encoding the nuclease; at least one guide RNA (gRNA) complementary to at least a portion of a target nucleic acid or a nucleic acid comprising a sequence encoding the at least one gRNA; and a nucleic acid encoding the recombinant receptor. In some embodiments, the methods comprise introducing into the cell a nuclease comprising an amino acid sequence having at least 70% identity, at least 80% identity, at least 90% identity, at least 95% identity or at least 99% identity to any of SEQ ID NOs: 1-23, 26-29, or 32-236 or a nucleic acid molecule comprising a sequence encoding the nuclease; at least one guide RNA (gRNA) complementary to at least a portion of a target nucleic acid or a nucleic acid comprising a sequence encoding the at least one gRNA; and a nucleic acid encoding the recombinant receptor. In some embodiments, the methods comprise introducing into the cell a nuclease comprising an amino acid sequence having about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or 100% identity to any of SEQ ID NOs: 1-23, 26-29, or 32-236 or a nucleic acid molecule comprising a sequence encoding the nuclease; at least one guide RNA (gRNA) complementary to at least a portion of a target nucleic acid or a nucleic acid comprising a sequence encoding the at least one gRNA; and a nucleic acid encoding the recombinant receptor.

In some embodiments, the nucleic acid encoding the recombinant receptor is integrated into genomic DNA of the cell.

In some embodiments, the recombinant receptor is a T cell receptor (TCR) or a chimeric antigen receptor (CAR). In some embodiments, the system and the recombinant receptor are encoded by separate nucleic acids.

In some embodiments, the cell is a T cell. In some embodiments, the T cell is from a subject. In some embodiments, the T cell is expanded in vitro.

Also provided are T cells comprising a disclosed nuclease, composition or system and a recombinant receptor or a nucleic acid encoding the recombinant receptor. In some embodiments, the T cell is from a subject.

Provided herein are methods for modifying a target nucleic acid in a plant. In some embodiments, the methods comprise providing to the plant, or a plant cell, seed, fruit, plant part, or propagation material of the plant an engineered nuclease, as disclosed herein, or a nucleic acid encoding thereof, and at least on gRNA complementary to at least a portion of the target nucleic acid or a nucleic acid comprising a sequence encoding the at least one gRNA. In some embodiments, the methods comprise providing to the plant, or a plant cell, seed, fruit, plant part, or propagation material of the plant a nuclease comprising an amino acid sequence having at least 70% identity, at least 80% identity, at least 90% identity, at least 95% identity or at least 99% identity to any of SEQ ID NOs: 1-23, 26-29, or 32-236 or a nucleic acid molecule comprising a sequence encoding the nuclease; and at least one guide RNA (gRNA) complementary to at least a portion of the target nucleic acid or a nucleic acid comprising a sequence encoding the at least one gRNA. In some embodiments, the methods comprise providing to the plant, or a plant cell, seed, fruit, plant part, or propagation material of the plant a nuclease comprising an amino acid sequence having about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or 100% identity to any of SEQ ID NOs: 1-23, 26-29, or 32-236 or a nucleic acid molecule comprising a sequence encoding the nuclease; and at least one guide RNA (gRNA) complementary to at least a portion of the target nucleic acid or a nucleic acid comprising a sequence encoding the at least one gRNA.

In some embodiments, the methods further comprise providing to the plant a donor polynucleotide. In some embodiments, the nucleic acid encodes a gene product.

In some embodiments, the plant is a grain crop, a fruit crop, a forage crop, a root vegetable crop, a leafy vegetable crop, a flowering plant, a conifer, an oil crop, a plant used in phytoremediation, an industrial crop, a medicinal crop, or a laboratory model plant.

In some embodiments, the nucleic acid molecule comprising a sequence encoding the nuclease or engineered nuclease and the at least one gRNA or the nucleic acid encoding the at least one guide RNA are provided via Agrobacterium-mediated transformation.

In some embodiments, the method confers one or more of the following traits to the plant or a plant cell, seed, fruit, plant part, or propagation material of the plant: herbicide tolerance, drought tolerance, male sterility, insect resistance, abiotic stress tolerance, modified fatty acid metabolism, modified carbohydrate metabolism, modified seed yield, modified oil percent, modified protein content, disease resistance, cold and frost tolerance, improved taste, increased germination, increased micronutrient uptake, improved flower longevity, modified fragrance, modified nutritional value, modified fruit or flower size or number, modified growth, and modified plant size.

Provided herein are method for treating a disease or disorder in a subject. In some embodiments, the methods comprise administering to the subject an engineered nuclease as disclosed herein, or a nucleic acid encoding thereof, and at least on gRNA complementary to at least a portion of the target nucleic acid or a nucleic acid comprising a sequence encoding the at least one gRNA, or cell as described herein. In some embodiments, the methods comprise administering to the subject: a nuclease comprising an amino acid sequence having at least 70% identity, at least 80% identity, at least 90% identity, at least 95% identity or at least 99% identity to any of SEQ ID NOs: 1-23, 26-29, or 32-236 or a nucleic acid molecule comprising a sequence encoding the nuclease; and at least one guide RNA (gRNA) complementary to at least a portion of a target nucleic acid or a nucleic acid comprising a sequence encoding the at least one gRNA; or a cell comprising a recombinant receptor or a nucleic acid encoding the recombinant receptor, the nuclease, or a nucleic acid encoding thereof, and at least one gRNA, or a nucleic acid encoding thereof. In some embodiments, the subject is a human. In some embodiments, the methods comprise administering to the subject: a nuclease comprising an amino acid sequence having about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or 100% identity to any of SEQ ID NOs: 1-23, 26-29, or 32-236 or a nucleic acid molecule comprising a sequence encoding the nuclease; and at least one guide RNA (gRNA) complementary to at least a portion of a target nucleic acid or a nucleic acid comprising a sequence encoding the at least one gRNA

In some embodiments, the nucleic acid molecule encoding each one or both of the nuclease and the at least one gRNA comprises a messenger RNA, a vector, or a combination thereof. In some embodiments, the nuclease and the at least one gRNA are encoded on a single nucleic acid. In some embodiments, the amino acid sequence has less than 50% sequence identity with SEQ ID NO. 24. In some embodiments, the nuclease further comprises a nuclear localization sequence (NLS).

In some embodiments, the at least one gRNA comprises a non-naturally occurring gRNA. In some embodiments, the at least one gRNA is encoded in a CRISPR RNA array.

In some embodiments, the cell is a T cell. In some embodiments, the T cell is from a subject. In some embodiments, the T cell is expanded in vitro. In some embodiments, the nucleic acid encoding the recombinant receptor is integrated into genomic DNA of the cell.

In some embodiments, the target nucleic acid is a disease-associated gene.

In some embodiments, the methods further comprise administering a donor polynucleotide. In some embodiments, the donor polynucleotide comprises a therapeutic protein, functional gene product, or a combination thereof.

In some embodiments, the methods further comprise administering a therapeutic agent.

Kits comprising any or all of the components of the compositions or systems described herein are also provided. In some embodiments, the kit further comprises one or more reagent, shipping and/or packaging containers, one or more buffers, a delivery device, instructions, software, a computing device, or a combination thereof.

Other aspects and embodiments of the disclosure will be apparent in light of the following detailed description.

### BRIEF DESCRIPTION OF THE DRAWINGS

FIG. 1 is vector diagrams of an exemplary nuclease expression vector and an exemplary targeting guide vector.

FIG. 2 is an image of an agarose gel of nuclease activity of a lysate.

FIGS. 3A and 3B show crRNA stem-loop modifications (FIG. 3A) and a graph of editing efficiencies of NUX019 targeting FANCF1 (FIG. 3B).

FIG. 4 shows crRNA spacer length and sequences (SEQ ID NO: 410-415) (top) and a graph of editing efficiencies of NUX019 targeting DNMT1-3 (bottom).

FIG. 5 shows results and profiling of cut sites resulting from nuclease activity on double stranded DNA (SEQ ID NO: 416).

FIG. 6 shows exemplary positioning of nuclear localization sites (NLS) and other protein tags.

FIG. 7 is a graph of editing efficiencies for NUX019 multiplex guide RNA targeting.

FIG. 8 is a graph of editing efficiencies for NUX019 with guide RNA mismatches at the indicated positions.

FIG. 9 is a graph of editing efficiencies for NUX019 and AsCas12a for direct repeats as indicated.

FIG. 10 shows the editing efficiency of NUX019, NUX063 and NUX082 with on-target and mismatched MM5 and MM9 DNMT1 guides in HEK293T cells.

FIGS. 11A-11C show the complete mismatch panel for the DNMT1 spacer for three nucleases: NUX019 (FIG. 11A), NUX063 (FIG. 11B), and NUX082 (FIG. 11C).

FIG. 12 shows the editing of DNMT1 by NUX019 with different repeats of C-myc NLS on the C-terminus.

FIG. 13 shows the editing of the DNMT1 gene (on target) or a mismatched DNMT1 guide (Mismatch 8) by NUX063 mRNA transfected into HEK293T cells.

FIG. 14 shows the editing of primary mouse hepatocytes by NUX063 mRNA and guides targeting either TTR or PCSK9 delivered by LNP.

FIG. 15 shows the editing of mouse livers via systemic LNP delivery of NUX063. mRNA of NUX063 and a guide targeting the TTR gene were systemically delivered via LNP to mice.

FIG. 16 shows the dose response of NUX019 or NUX063 RNP nucleofected into HEK293T cells targeting the DNMT1 gene.

FIG. 17A shows the editing of the TCRA, PD1, and β2M loci in primary human T cells by NUX138 RNP. FIG. 17B shows the staining of cell surface proteins stained with antibodies targeting TCRA, PD-1, or β2M and analyzed by flow cytometry. NT=non-edited T cells.

FIG. 18 is a qPCR analysis of CD-19 CAR expression in primary T cells.

FIG. 19 is a PCR analysis of CD-19 CAR integration in genomic DNA.

FIG. 20 shows the reduction of CD-19 positive NALM6 cancer cells by treatment with engineered CD-19 CAR T cells. Control=untreated NALM6 cells, CD-19 CAR T=NALM6 cells treated with CAR T.

FIG. 21 shows the editing efficiency of NUX058, NUX059, NUX069, NUX079, NUX081 and NUX063.

FIG. 22 is a PAM analysis of NUX019 and engineered NUX063 and NUX082 showing increases in PAM diversity.

FIG. 23 shows NUX019 and NUX063 optimized with an OPT NLS compared against non-optimized versions for editing efficiency.

FIG. 24 is a graph of editing efficiencies various nucleases (NUX #indicated on x axis).

### DETAILED DESCRIPTION

The disclosed compositions, systems, kits, and methods comprise nucleases useful for nucleic acid modification. The disclosed nucleases allow for high precision gene editing with improved efficacy and safety for use in in vivo and ex vivo applications of eukaryotic (e.g., mammalian (e.g., human)) therapeutics, diagnostics, and research.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of cell and tissue culture, molecular biology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

Nucleic acid or amino acid sequence “identity,” as described herein, can be determined by comparing a nucleic acid or amino acid sequence of interest to a reference nucleic acid or amino acid sequence. The percent identity is the number of nucleotides or amino acid residues that are the same (e.g., that are identical) as between the sequence of interest and the reference sequence divided by the length of the longest sequence (e.g., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3×, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings. Trees and Sequences, Cambridge University Press, Cambridge UK (1997)).

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which it is naturally associated in nature and as found in nature, and/or the nucleic acid molecule or the polypeptide is associated with at least one other component with which it is not naturally associated in nature and/or that there is one or more changes in nucleic acid or amino acid sequence as compared with such sequence as it is found in nature.

A “vector” or “expression vector” is a replicon, such as plasmid, phage, virus, or cosmid, to which another DNA segment, e.g., an “insert,” may be attached or incorporated so as to bring about the replication of the attached segment in a cell.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. For example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

The term “contacting” as used herein refers to bring or put in contact, to be in or come into contact. The term “contact” as used herein refers to a state or condition of touching or of immediate or local proximity. Contacting a composition to a target destination, such as, but not limited to, an organ, tissue, cell, or tumor, may occur by any means of administration known to the skilled artisan.

As used herein, the terms “providing,” “administering,” and “introducing” are used interchangeably herein and refer to the placement of the composition or systems of the disclosure into a cell, organism, or subject by a method or route which results in at least partial localization to a desired site. The composition or systems can be administered by any appropriate route which results in delivery to a desired location in the cell, organism, or subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Engineered Nucleases

Advances and developments in CRISPR-Cas genome editing tools including nucleases and other Cas protein drive major advances in nucleic acid editing. Nucleic acid editing has many uses including in the diagnostics and therapeutics field. Such breadth is accompanied by a diversity of nucleic acid targets and environments in which to engineer editing activity. As such, there is a need for diverse and additional nucleases and associated methods that provide a toolbox for nucleic acid editing.

Disclosed herein are compositions that include nucleases (NUX) that have Cas-like activity. The disclosed engineered nucleases comprise a sequence having at least 70% identity (e.g., at least 75%, at least 80%, at least 85%, at least 90%, at least 93%, at least 95%, at least 98%, or at least 99%) to an amino acid sequence of SEQ ID NOs: 1-23, 26-29, or 32-236. The disclosed engineered nucleases comprise a sequence having about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or 100% identity to an amino acid sequence of SEQ ID NOs: 1-23, 26-29, or 32-236. In some embodiments, the engineered nuclease comprises a sequence having at least 90% identity (e.g., about 90%, about 91%, about 92%, about 93%, about 94%, about 95%, about 96%, about 97%, about 98%, about 99%, or 100%) an amino acid sequence of SEQ ID NOs: 1-23, 26-29, or 32-236. In certain embodiments, the engineered nuclease comprises an amino acid sequence of SEQ ID NOs: 1-23, 26-29, or 32-236.

In some embodiments, the amino acid sequence of the nuclease lacks identity or significant amino acid homology with certain known Cas nucleases. In some embodiments, the amino acid sequence of the nuclease lacks identity or significant amino acid homology with Cas12a (Cpf1) protein. In some embodiments, the amino acid sequence of the nuclease lacks identity or significant amino acid homology with a Cas9 such as SaCas9 or SpyCas9. In some embodiments, the amino acid sequence of the nuclease has less than 50%, less than 48%, less than 45%, less than 40%, less than 35% or less than 34% with Cas12a (Cpf1) protein, SaCas9 or SpyCas9 proteins. In some embodiments, the amino acid sequence of the nuclease has less than 50%, less than 48%, less than 45%, less than 40%, less than 35% or less than 34% with SEQ ID NO: 24.

Any of the nucleases described herein (e.g., nucleases having at least 75%, at least 80%, at least 85%, at least 90%, at least 93%, at least 95%, at least 98%, or at least 99% to an amino acid sequence of SEQ ID NOs: 1-23, 26-29, or 32-236) may comprise one or more (e.g., 1, 2, 3, 4, 5, 6, 7, 8, 9, 10, 15, 20, 25, 50, 100, 150, 200, etc.) amino acid substitutions. An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free-OH can be maintained, and glutamine for asparagine such that a free-NH: can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In some embodiments, an engineered nuclease comprises one or more amino acid substitutions and has an amino acid sequence having at least 70% identity (e.g., at least 75%, at least 80%, at least 85%, at least 90%, at least 93%, at least 95%, at least 98%, or at least 99% identity) to an amino acid sequence of SEQ ID NOs: 1-23 or 32-35. In some embodiments, an engineered nuclease comprises one or more amino acid substitutions and has an amino acid sequence having about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or 100% identity to an amino acid sequence of SEQ ID NOs: 1-23 or 32-35. In some embodiments, a nuclease comprises one or more amino acid substitutions and comprises an amino acid sequence of SEQ ID NOs: 26-29 or 36-236.

In some embodiments, the engineered nuclease comprises an amino acid sequence having at least 90% (e.g., at least 90%, at least 91%, at least 92%, at least 93%, at least 94%, at least 95%, at least 96%, at least 97%, at least 98%, at least 99%) to SEQ ID NO: 8 with one or more amino acid replacements or substitutions corresponding to positions selected from the group consisting of 174, 283, 604, and 598. In some embodiments, the engineered nuclease comprises a replacement or substitution at position 174. In some embodiments, the engineered nuclease comprises a replacement or substitution at position 283. In some embodiments, the engineered nuclease comprises a replacement or substitution at position 604. In some embodiments, the engineered nuclease comprises a replacement or substitution at position 598.

In some embodiments, the engineered nuclease comprises two amino acid replacements at positions selected from the group consisting of 174, 283, 604, and 598, in reference to SEQ ID NO: 8. In some embodiments, the engineered nuclease comprises a replacement or substitution at positions 174 and 283. In some embodiments, the engineered nuclease comprises a replacement or substitution at positions 174 and 604. In some embodiments, the engineered nuclease comprises a replacement or substitution at positions 174 and 598. In some embodiments, the engineered nuclease comprises a replacement or substitution at positions 283 and 604. In some embodiments, the engineered nuclease comprises a replacement or substitution at positions 283 and 598. In some embodiments, the engineered nuclease comprises a replacement or substitution at positions 604 and 598.

In some embodiments, the engineered nuclease comprises three amino acid replacements at positions selected from the group consisting of 174, 283, 604, and 598, in reference to SEQ ID NO: 8. In some embodiments, the engineered nuclease comprises a replacement or substitution at positions 174, 283 and 604. In some embodiments, the engineered nuclease comprises a replacement or substitution at positions 174, 283 and 598. In some embodiments, the engineered nuclease comprises a replacement or substitution at positions 283, 604 and 598. In some embodiments, the engineered nuclease comprises a replacement or substitution at positions 174, 604 and 598.

In some embodiments, the engineered nuclease comprises a replacement or substitution at positions 174, 283, 604 and 598, in reference to SEQ ID NO: 8.

In some embodiments, the amino acid sequence lacks one or more of the following features: E at position 174, N at position 283, K at position 604 and S at position 598. In some embodiments, the replacement at position 174 is an E174R replacement. In some embodiments, the replacement at position 283 is a N283A replacement. In some embodiments, the replacement at position 598 is an S598R replacement. In some embodiments, the replacement at 604 is a K604R replacement.

In some embodiments, the one or more amino acid replacements is selected from the group consisting of E174R, N283A, S598R, and K604R, in reference to SEQ ID NO: 8. In some embodiments, the one or more amino acid replacements comprises an E174R replacement, and N283 A replacement, an S598R replacement, or an K604R replacement, in reference to SEQ ID NO: 8.

In some embodiments, an engineered nuclease comprises an amino acid sequence having at least 90% (e.g., at least 90%, at least 91%, at least 92%, at least 93%, at least 94%, at least 95%, at least 96%, at least 97%, at least 98%, at least 99%) to SEQ ID NO: 1 with one or more amino acid replacements or substitutions corresponding to positions selected from the group consisting of 139, 231, 491, 496, 502 and 779. In some embodiments, the engineered nuclease comprises a replacement or substitution at position 139. In some embodiments, the engineered nuclease comprises a replacement or substitution at position 231. In some embodiments, the engineered nuclease comprises a replacement or substitution at position 491. In some embodiments, the engineered nuclease comprises a replacement or substitution at position 496. In some embodiments, the engineered nuclease comprises a replacement or substitution at position 502. In some embodiments, the engineered nuclease comprises a replacement or substitution at position 779.

In some embodiments, the engineered nuclease comprises two amino acid replacements at positions selected from the group consisting of 139, 231, 491, 496, 502 and 779, in reference to SEQ ID NO: 1. In some embodiments, the engineered nuclease comprises a replacement or substitution at positions 139 and 231. In some embodiments, the engineered nuclease comprises a replacement or substitution at positions 139 and 491. In some embodiments, the engineered nuclease comprises a replacement or substitution at positions 139 and 496. In some embodiments, the engineered nuclease comprises a replacement or substitution at positions 139 and 502. In some embodiments, the engineered nuclease comprises a replacement or substitution at positions 139 and 779. In some embodiments, the engineered nuclease comprises a replacement or substitution at positions 231 and 491. In some embodiments, the engineered nuclease comprises a replacement or substitution at positions 231 and 496. In some embodiments, the engineered nuclease comprises a replacement or substitution at positions 231 and 502. In some embodiments, the engineered nuclease comprises a replacement or substitution at positions 231 and 779. In some embodiments, the engineered nuclease comprises a replacement or substitution at positions 491 and 496. In some embodiments, the engineered nuclease comprises a replacement or substitution at positions 491 and 502. In some embodiments, the engineered nuclease comprises a replacement or substitution at positions 491 and 779. In some embodiments, the engineered nuclease comprises a replacement or substitution at positions 496 and 502. In some embodiments, the engineered nuclease comprises a replacement or substitution at positions 496 and 779. In some embodiments, the engineered nuclease comprises a replacement or substitution at positions 502 and 779.

In some embodiments, the engineered nuclease comprises three amino acid replacements at positions selected from the group consisting of 139, 231, 491, 496, 502 and 779, in reference to SEQ ID NO: 1. In some embodiments, the engineered nuclease comprises a replacement or substitution at positions 139, 231, and 491. In some embodiments, the engineered nuclease comprises a replacement or substitution at positions 139, 231, and 496. In some embodiments, the engineered nuclease comprises a replacement or substitution at positions 139, 231, and 502. In some embodiments, the engineered nuclease comprises a replacement or substitution at positions 139, 231, and 779. In some embodiments, the engineered nuclease comprises a replacement or substitution at positions 139, 491, and 496. In some embodiments, the engineered nuclease comprises a replacement or substitution at positions 139, 491, and 502. In some embodiments, the engineered nuclease comprises a replacement or substitution at positions 139, 491, and 779. In some embodiments, the engineered nuclease comprises a replacement or substitution at positions 139, 496, and 502. In some embodiments, the engineered nuclease comprises a replacement or substitution at positions 139, 496, and 779. In some embodiments, the engineered nuclease comprises a replacement or substitution at positions 139, 502, and 779. In some embodiments, the engineered nuclease comprises a replacement or substitution at positions 231, 491, and 496. In some embodiments, the engineered nuclease comprises a replacement or substitution at positions 231, 491, and 502. In some embodiments, the engineered nuclease comprises a replacement or substitution at positions 231, 491, and 779. In some embodiments, the engineered nuclease comprises a replacement or substitution at positions 231, 496, and 502. In some embodiments, the engineered nuclease comprises a replacement or substitution at positions 231, 496, and 779. In some embodiments, the engineered nuclease comprises a replacement or substitution at positions 231, 502, and 779. In some embodiments, the engineered nuclease comprises a replacement or substitution at positions 491, 496, and 502. In some embodiments, the engineered nuclease comprises a replacement or substitution at positions 491, 496, and 779. In some embodiments, the engineered nuclease comprises a replacement or substitution at positions 491, 502, and 779. In some embodiments, the engineered nuclease comprises a replacement or substitution at positions 496, 502 and 779.

In some embodiments, the amino acid sequence lacks one or more of the following features: S at position 139, N at position 231, N at position 491, G at position 496, K at position 502 and A at position 779. In some embodiments, the replacement at 779 is a A779L replacement. In some embodiments, the replacement at position 139 is an S139R replacement. In some embodiments, the replacement at position 231 is a N231A replacement. In some embodiments, the replacement at position 491 is an N491R replacement. In some embodiments, the replacement at 496 is a G496R replacement. In some embodiments, the replacement at 502 is a K502R replacement.

In some embodiments, the one or more amino acid replacements is selected from the group consisting of S139R, N231A, N491R, G496R, K502R and A779L, in reference to SEQ ID NO: 1. In some embodiments, the one or more amino acid replacements comprises an S139R replacement, and N231A replacement, an N491R replacement, an G496R replacement, an K502R replacement, or an A779L replacement, in reference to SEQ ID NO: 1.

In some embodiments, an engineered nuclease comprises an amino acid sequence having at least 90% (e.g., at least 90%, at least 91%, at least 92%, at least 93%, at least 94%, at least 95%, at least 96%, at least 97%, at least 98%, at least 99%) to SEQ ID NO: 2 with one or more amino acid replacements or substitutions corresponding to positions selected from the group consisting of 139, 231, 496, 491, and 502. In some embodiments, the engineered nuclease comprises a replacement or substitution at position 139. In some embodiments, the engineered nuclease comprises a replacement or substitution at position 231. In some embodiments, the engineered nuclease comprises a replacement or substitution at position 491. In some embodiments, the engineered nuclease comprises a replacement or substitution at position 496. In some embodiments, the engineered nuclease comprises a replacement or substitution at position 502.

In some embodiments, the engineered nuclease comprises two amino acid replacements at positions selected from the group consisting of 139, 231, 491, 496, and 502, in reference to SEQ ID NO: 2. In some embodiments, the engineered nuclease comprises a replacement or substitution at positions 139 and 231. In some embodiments, the engineered nuclease comprises a replacement or substitution at positions 139 and 491. In some embodiments, the engineered nuclease comprises a replacement or substitution at positions 139 and 496. In some embodiments, the engineered nuclease comprises a replacement or substitution at positions 139 and 502. In some embodiments, the engineered nuclease comprises a replacement or substitution at positions 231 and 491. In some embodiments, the engineered nuclease comprises a replacement or substitution at positions 231 and 496. In some embodiments, the engineered nuclease comprises a replacement or substitution at positions 231 and 502. In some embodiments, the engineered nuclease comprises a replacement or substitution at positions 491 and 496. In some embodiments, the engineered nuclease comprises a replacement or substitution at positions 491 and 502. In some embodiments, the engineered nuclease comprises a replacement or substitution at positions 496 and 502.

In some embodiments, the engineered nuclease comprises three amino acid replacements at positions selected from the group consisting of 139, 231, 496, 491, and 502, in reference to SEQ ID NO: 2. In some embodiments, the engineered nuclease comprises a replacement or substitution at positions 139, 231, and 491. In some embodiments, the engineered nuclease comprises a replacement or substitution at positions 139, 231, and 496. In some embodiments, the engineered nuclease comprises a replacement or substitution at positions 139, 231, and 502. In some embodiments, the engineered nuclease comprises a replacement or substitution at positions 139, 491, and 496. In some embodiments, the engineered nuclease comprises a replacement or substitution at positions 139, 491, and 502. In some embodiments, the engineered nuclease comprises a replacement or substitution at positions 139, 496, and 502. In some embodiments, the engineered nuclease comprises a replacement or substitution at positions 231, 491, and 496. In some embodiments, the engineered nuclease comprises a replacement or substitution at positions 231, 491, and 502. In some embodiments, the engineered nuclease comprises a replacement or substitution at positions 231, 496, and 502. In some embodiments, the engineered nuclease comprises a replacement or substitution at positions 491, 496, and 502.

In some embodiments, the amino acid sequence lacks one or more of the following features: S at position 139, N at position 231, N at position 491, D at position 496, and Y at position 502. In some embodiments, the replacement at position 139 is an S139R replacement. In some embodiments, the replacement at position 231 is a N231A replacement. In some embodiments, the replacement at position 491 is an N491R replacement. In some embodiments, the replacement at 496 is a D496R replacement. In some embodiments, the replacement at 502 is a Y502R replacement.

In some embodiments, the one or more amino acid replacements is selected from the group consisting of S139R, N231A, N491R, D496R, and Y502R, in reference to SEQ ID NO: 2. In some embodiments, the one or more amino acid replacements comprises an S139R replacement, an N231A replacement, an N491R replacement, a D496R replacement, or a Y502R replacement, in reference to SEQ ID NO: 2.

In some embodiments, an engineered nuclease comprises an amino acid sequence having at least 90% (e.g., at least 90%, at least 91%, at least 92%, at least 93%, at least 94%, at least 95%, at least 96%, at least 97%, at least 98%, at least 99%) to SEQ ID NO: 3 with one or more amino acid replacements or substitutions corresponding to positions selected from the group consisting of 175, 272, 544, 549, and 555. In some embodiments, the engineered nuclease comprises a replacement or substitution at position 175. In some embodiments, the engineered nuclease comprises a replacement or substitution at position 272. In some embodiments, the engineered nuclease comprises a replacement or substitution at position 544. In some embodiments, the engineered nuclease comprises a replacement or substitution at position 549. In some embodiments, the engineered nuclease comprises a replacement or substitution at position 555.

In some embodiments, the engineered nuclease comprises two amino acid replacements at positions selected from the group consisting of 175, 272, 544, 549, and 555, in reference to SEQ ID NO: 3. In some embodiments, the engineered nuclease comprises a replacement or substitution at positions 175 and 272. In some embodiments, the engineered nuclease comprises a replacement or substitution at positions 175 and 544. In some embodiments, the engineered nuclease comprises a replacement or substitution at positions 175 and 549. In some embodiments, the engineered nuclease comprises a replacement or substitution at positions 175 and 255. In some embodiments, the engineered nuclease comprises a replacement or substitution at positions 272 and 544. In some embodiments, the engineered nuclease comprises a replacement or substitution at positions 272 and 549. In some embodiments, the engineered nuclease comprises a replacement or substitution at positions 272 and 555. In some embodiments, the engineered nuclease comprises a replacement or substitution at positions 544 and 549. In some embodiments, the engineered nuclease comprises a replacement or substitution at positions 544 and 555. In some embodiments, the engineered nuclease comprises a replacement or substitution at positions 549 and 555.

In some embodiments, the engineered nuclease comprises three amino acid replacements at positions selected from the group consisting of 175, 272, 544, 549, and 555, in reference to SEQ ID NO: 3. In some embodiments, the engineered nuclease comprises a replacement or substitution at positions 175, 272, and 544. In some embodiments, the engineered nuclease comprises a replacement or substitution at positions 175, 272, and 549. In some embodiments, the engineered nuclease comprises a replacement or substitution at positions 175, 272, and 555. In some embodiments, the engineered nuclease comprises a replacement or substitution at positions 175, 544, and 549. In some embodiments, the engineered nuclease comprises a replacement or substitution at positions 175, 544, and 555. In some embodiments, the engineered nuclease comprises a replacement or substitution at positions 175, 549, and 555. In some embodiments, the engineered nuclease comprises a replacement or substitution at positions 272, 544, and 549. In some embodiments, the engineered nuclease comprises a replacement or substitution at positions 272, 544, and 555. In some embodiments, the engineered nuclease comprises a replacement or substitution at positions 272, 549, and 555. In some embodiments, the engineered nuclease comprises a replacement or substitution at positions 544, 549, and 555.

In some embodiments, the amino acid sequence lacks one or more of the following features: E at position 175, N at position 272, N at position 544, T at position 549, and K at position 555. In some embodiments, the replacement at position 175 is an E175R replacement. In some embodiments, the replacement at position 272 is a N272A replacement. In some embodiments, the replacement at position 544 is an N544R replacement. In some embodiments, the replacement at 549 is a T549R replacement. In some embodiments, the replacement at 555 is a K555R replacement.

In some embodiments, the one or more amino acid replacements is selected from the group consisting of E175R, N272A, N544R, T549R, and K555R, in reference to SEQ ID NO: 3. In some embodiments, the one or more amino acid replacements comprises an E175R replacement, an N272A replacement, an N544R replacement, a T549R replacement, or a K555R replacement, in reference to SEQ ID NO: 3.

In some embodiments, an engineered nuclease comprises an amino acid sequence having at least 90% (e.g., at least 90%, at least 91%, at least 92%, at least 93%, at least 94%, at least 95%, at least 96%, at least 97%, at least 98%, at least 99%) to SEQ ID NO: 4 with one or more amino acid replacements or substitutions corresponding to positions selected from the group consisting of 179, 261, 544, 549, and 555. In some embodiments, the engineered nuclease comprises a replacement or substitution at position 179. In some embodiments, the engineered nuclease comprises a replacement or substitution at position 261. In some embodiments, the engineered nuclease comprises a replacement or substitution at position 544. In some embodiments, the engineered nuclease comprises a replacement or substitution at position 549. In some embodiments, the engineered nuclease comprises a replacement or substitution at position 555.

In some embodiments, the engineered nuclease comprises two amino acid replacements at positions selected from the group consisting of 179, 261, 544, 549, and 555, in reference to SEQ ID NO: 4. In some embodiments, the engineered nuclease comprises a replacement or substitution at positions 179 and 261. In some embodiments, the engineered nuclease comprises a replacement or substitution at positions 179 and 544. In some embodiments, the engineered nuclease comprises a replacement or substitution at positions 179 and 549. In some embodiments, the engineered nuclease comprises a replacement or substitution at positions 179 and 255. In some embodiments, the engineered nuclease comprises a replacement or substitution at positions 261 and 544. In some embodiments, the engineered nuclease comprises a replacement or substitution at positions 261 and 549. In some embodiments, the engineered nuclease comprises a replacement or substitution at positions 261 and 555. In some embodiments, the engineered nuclease comprises a replacement or substitution at positions 544 and 549. In some embodiments, the engineered nuclease comprises a replacement or substitution at positions 544 and 555. In some embodiments, the engineered nuclease comprises a replacement or substitution at positions 549 and 555.

In some embodiments, the engineered nuclease comprises three amino acid replacements at positions selected from the group consisting of 179, 261, 544, 549, and 555, in reference to SEQ ID NO: 4. In some embodiments, the engineered nuclease comprises a replacement or substitution at positions 179, 261, and 544. In some embodiments, the engineered nuclease comprises a replacement or substitution at positions 179, 261, and 549. In some embodiments, the engineered nuclease comprises a replacement or substitution at positions 179, 261, and 555. In some embodiments, the engineered nuclease comprises a replacement or substitution at positions 179, 544, and 549. In some embodiments, the engineered nuclease comprises a replacement or substitution at positions 179, 544, and 555. In some embodiments, the engineered nuclease comprises a replacement or substitution at positions 179, 549, and 555. In some embodiments, the engineered nuclease comprises a replacement or substitution at positions 261, 544, and 549. In some embodiments, the engineered nuclease comprises a replacement or substitution at positions 261, 544, and 555. In some embodiments, the engineered nuclease comprises a replacement or substitution at positions 261, 549, and 555. In some embodiments, the engineered nuclease comprises a replacement or substitution at positions 544, 549, and 555.

In some embodiments, the amino acid sequence lacks one or more of the following features: K at position 179, N at position 261, Y at position 544, G at position 549, and K at position 555. In some embodiments, the replacement at position 179 is an K179R replacement. In some embodiments, the replacement at position 261 is a N261A replacement. In some embodiments, the replacement at position 544 is an N544R replacement. In some embodiments, the replacement at 549 is a T549R replacement. In some embodiments, the replacement at 555 is a K555R replacement. In some embodiments, the one or more amino acid replacements is selected from the group consisting of K179R, N261A, N544R, T549R, and K555R, in reference to SEQ ID NO: 4. In some embodiments, the one or more amino acid replacements comprises an K179R replacement, an N261A replacement, an N544R replacement, a T549R replacement, or a K555R replacement, in reference to SEQ ID NO. 4.

In some embodiments, an engineered nuclease comprises an amino acid sequence with at least 90% (e.g., at least 90%, at least 91%, at least 92%, at least 93%, at least 94%, at least 95%, at least 96%, at least 97%, at least 98%, at least 99%) to SEQ ID NO: 5 with one or more amino acid replacements corresponding to positions selected from the group consisting of 161, 261, 534, 539, 545, and 803. In some embodiments, the engineered nuclease comprises a replacement or substitution at position 161. In some embodiments, the engineered nuclease comprises a replacement or substitution at position 261. In some embodiments, the engineered nuclease comprises a replacement or substitution at position 534. In some embodiments, the engineered nuclease comprises a replacement or substitution at position 539. In some embodiments, the engineered nuclease comprises a replacement or substitution at position 803.

In some embodiments, the engineered nuclease comprises two amino acid replacements at positions selected from the group consisting of 161, 261, 534, 539, 545, and 803, in reference to SEQ ID NO: 5. In some embodiments, the engineered nuclease comprises a replacement or substitution at positions 161 and 261. In some embodiments, the engineered nuclease comprises a replacement or substitution at positions 161 and 534. In some embodiments, the engineered nuclease comprises a replacement or substitution at positions 161 and 539. In some embodiments, the engineered nuclease comprises a replacement or substitution at positions 161 and 545. In some embodiments, the engineered nuclease comprises a replacement or substitution at positions 161 and 803. In some embodiments, the engineered nuclease comprises a replacement or substitution at positions 261 and 534. In some embodiments, the engineered nuclease comprises a replacement or substitution at positions 261 and 539. In some embodiments, the engineered nuclease comprises a replacement or substitution at positions 261 and 545. In some embodiments, the engineered nuclease comprises a replacement or substitution at positions 261 and 803. In some embodiments, the engineered nuclease comprises a replacement or substitution at positions 534 and 539. In some embodiments, the engineered nuclease comprises a replacement or substitution at positions 534 and 545. In some embodiments, the engineered nuclease comprises a replacement or substitution at positions 534 and 803. In some embodiments, the engineered nuclease comprises a replacement or substitution at positions 539 and 545. In some embodiments, the engineered nuclease comprises a replacement or substitution at positions 539 and 803. In some embodiments, the engineered nuclease comprises a replacement or substitution at positions 545 and 803.

In some embodiments, the engineered nuclease comprises three amino acid replacements at positions selected from the group consisting of 161, 261, 534, 539, 545, and 803, in reference to SEQ ID NO: 5. In some embodiments, the engineered nuclease comprises a replacement or substitution at positions 161, 261, and 534. In some embodiments, the engineered nuclease comprises a replacement or substitution at positions 161, 261, and 539. In some embodiments, the engineered nuclease comprises a replacement or substitution at positions 161, 261, and 545. In some embodiments, the engineered nuclease comprises a replacement or substitution at positions 161, 261, and 803. In some embodiments, the engineered nuclease comprises a replacement or substitution at positions 161, 534, and 539. In some embodiments, the engineered nuclease comprises a replacement or substitution at positions 161, 534, and 545. In some embodiments, the engineered nuclease comprises a replacement or substitution at positions 161, 534, and 803. In some embodiments, the engineered nuclease comprises a replacement or substitution at positions 161, 539, and 545. In some embodiments, the engineered nuclease comprises a replacement or substitution at positions 161, 539, and 803. In some embodiments, the engineered nuclease comprises a replacement or substitution at positions 161, 545, and 803. In some embodiments, the engineered nuclease comprises a replacement or substitution at positions 261, 534, and 539. In some embodiments, the engineered nuclease comprises a replacement or substitution at positions 261, 534, and 545. In some embodiments, the engineered nuclease comprises a replacement or substitution at positions 261, 534, and 803. In some embodiments, the engineered nuclease comprises a replacement or substitution at positions 261, 539, and 545. In some embodiments, the engineered nuclease comprises a replacement or substitution at positions 261, 539, and 803. In some embodiments, the engineered nuclease comprises a replacement or substitution at positions 261, 545, and 803. In some embodiments, the engineered nuclease comprises a replacement or substitution at positions 534, 539, and 545. In some embodiments, the engineered nuclease comprises a replacement or substitution at positions 534, 539, and 803. In some embodiments, the engineered nuclease comprises a replacement or substitution at positions 534, 545, and 803. In some embodiments, the engineered nuclease comprises a replacement or substitution at positions 539, 545 and 803.

In some embodiments, the amino acid sequence lacks one or more of the following features: T at position 161, N at position 261, C at position 534, N at position 539, K at position 545, G at position 549, and Q at position 803. In some embodiments, the replacement at position 161 is an T161R replacement. In some embodiments, the replacement at position 261 is a N261A replacement. In some embodiments, the replacement at position 534 is an C534R replacement. In some embodiments, the replacement at 539 is a NS39R replacement. In some embodiments, the replacement at 545 is a K545R replacement. In some embodiments, the replacement at 803 is a Q803L replacement.

In some embodiments, the one or more amino acid replacements is selected from the group consisting of T161R, N261A, C534R, N539R, K545R and Q803L, in reference to SEQ ID NO: 5. In some embodiments, the one or more amino acid replacements comprises an T161R replacement, and N261A replacement, an C534R replacement, an N539R replacement, an K545R replacement, or an Q803L replacement, in reference to SEQ ID NO: 5.

In some embodiments, an engineered nuclease comprises an amino acid sequence with at least 90% (e.g., at least 90%, at least 91%, at least 92%, at least 93%, at least 94%, at least 95%, at least 96%, at least 97%, at least 98%, at least 99%) to SEQ ID NO: 6 with one or more amino acid replacements corresponding to positions selected from the group consisting of 137, 242, 523, 528, and 534. In some embodiments, the engineered nuclease comprises a replacement or substitution at position 137. In some embodiments, the engineered nuclease comprises a replacement or substitution at position 242. In some embodiments, the engineered nuclease comprises a replacement or substitution at position 523. In some embodiments, the engineered nuclease comprises a replacement or substitution at position 528. In some embodiments, the engineered nuclease comprises a replacement or substitution at position 534.

In some embodiments, the engineered nuclease comprises two amino acid replacements at positions selected from the group consisting of 137, 242, 523, 528, and 534, in reference to SEQ ID NO: 6. In some embodiments, the engineered nuclease comprises a replacement or substitution at positions 137 and 242. In some embodiments, the engineered nuclease comprises a replacement or substitution at positions 137 and 523. In some embodiments, the engineered nuclease comprises a replacement or substitution at positions 137 and 528. In some embodiments, the engineered nuclease comprises a replacement or substitution at positions 137 and 534. In some embodiments, the engineered nuclease comprises a replacement or substitution at positions 242 and 523. In some embodiments, the engineered nuclease comprises a replacement or substitution at positions 242 and 528. In some embodiments, the engineered nuclease comprises a replacement or substitution at positions 242 and 534. In some embodiments, the engineered nuclease comprises a replacement or substitution at positions 523 and 528. In some embodiments, the engineered nuclease comprises a replacement or substitution at positions 523 and 534. In some embodiments, the engineered nuclease comprises a replacement or substitution at positions 528 and 534.

In some embodiments, the engineered nuclease comprises three amino acid replacements at positions selected from the group consisting of 137, 242, 523, 528, and 534, in reference to SEQ ID NO: 6. In some embodiments, the engineered nuclease comprises a replacement or substitution at positions 137, 242, and 523. In some embodiments, the engineered nuclease comprises a replacement or substitution at positions 137, 242, and 528. In some embodiments, the engineered nuclease comprises a replacement or substitution at positions 137, 242, and 534. In some embodiments, the engineered nuclease comprises a replacement or substitution at positions 137, 523, and 528. In some embodiments, the engineered nuclease comprises a replacement or substitution at positions 137, 523, and 534. In some embodiments, the engineered nuclease comprises a replacement or substitution at positions 137, 523, and 528. In some embodiments, the engineered nuclease comprises a replacement or substitution at positions 242, 523, and 528. In some embodiments, the engineered nuclease comprises a replacement or substitution at positions 242, 523, and 534. In some embodiments, the engineered nuclease comprises a replacement or substitution at positions 242, 528, and 534. In some embodiments, the engineered nuclease comprises a replacement or substitution at positions 523, 528, and 534.

In some embodiments, the amino acid sequence lacks one or more of the following features: T at position 137, N at position 242, N at position 523, N at position 528, and K at position 534. In some embodiments, the replacement at position 137 is an T137R replacement. In some embodiments, the replacement at position 242 is a N242A replacement. In some embodiments, the replacement at position 523 is an N523R replacement. In some embodiments, the replacement at 528 is a N528R replacement. In some embodiments, the replacement at 534 is a K534R replacement. In some embodiments, the one or more amino acid replacements is selected from the group consisting of T137R, N242A, N523R, N528R, and K534R, in reference to SEQ ID NO: 6. In some embodiments, the one or more amino acid replacements comprises an T137R replacement, an N242A replacement, an N523R replacement, a N528R replacement, or a K534R replacement, in reference to SEQ ID NO: 6.

In some embodiments, an engineered nuclease comprises an amino acid sequence with at least 90% or at least 95% identity to SEQ ID NO: 7 with one or more amino acid replacements corresponding to positions selected from the group consisting of 160, 265, 542, 547, 553, and 842. In some embodiments, the engineered nuclease comprises a replacement or substitution at position 160. In some embodiments, the engineered nuclease comprises a replacement or substitution at position 265. In some embodiments, the engineered nuclease comprises a replacement or substitution at position 542. In some embodiments, the engineered nuclease comprises a replacement or substitution at position 547. In some embodiments, the engineered nuclease comprises a replacement or substitution at position 553. In some embodiments, the engineered nuclease comprises a replacement or substitution at position 842.

In some embodiments, the engineered nuclease comprises two amino acid replacements at positions selected from the group consisting of 160, 265, 542, 547, 553, and 842, in reference to SEQ ID NO: 7. In some embodiments, the engineered nuclease comprises a replacement or substitution at positions 160 and 265. In some embodiments, the engineered nuclease comprises a replacement or substitution at positions 160 and 542. In some embodiments, the engineered nuclease comprises a replacement or substitution at positions 160 and 547. In some embodiments, the engineered nuclease comprises a replacement or substitution at positions 160 and 553. In some embodiments, the engineered nuclease comprises a replacement or substitution at positions 160 and 842. In some embodiments, the engineered nuclease comprises a replacement or substitution at positions 265 and 542. In some embodiments, the engineered nuclease comprises a replacement or substitution at positions 265 and 547. In some embodiments, the engineered nuclease comprises a replacement or substitution at positions 265 and 553. In some embodiments, the engineered nuclease comprises a replacement or substitution at positions 265 and 842. In some embodiments, the engineered nuclease comprises a replacement or substitution at positions 542 and 547. In some embodiments, the engineered nuclease comprises a replacement or substitution at positions 542 and 553. In some embodiments, the engineered nuclease comprises a replacement or substitution at positions 542 and 842. In some embodiments, the engineered nuclease comprises a replacement or substitution at positions 547 and 553. In some embodiments, the engineered nuclease comprises a replacement or substitution at positions 547 and 842. In some embodiments, the engineered nuclease comprises a replacement or substitution at positions 553 and 842.

In some embodiments, the engineered nuclease comprises three amino acid replacements at positions selected from the group consisting of 160, 265, 542, 547, 553, and 842, in reference to SEQ ID NO: 7. In some embodiments, the engineered nuclease comprises a replacement or substitution at positions 160, 265, and 542. In some embodiments, the engineered nuclease comprises a replacement or substitution at positions 160, 265, and 547. In some embodiments, the engineered nuclease comprises a replacement or substitution at positions 160, 265, and 553. In some embodiments, the engineered nuclease comprises a replacement or substitution at positions 160, 265, and 842. In some embodiments, the engineered nuclease comprises a replacement or substitution at positions 160, 542, and 547. In some embodiments, the engineered nuclease comprises a replacement or substitution at positions 160, 542, and 553. In some embodiments, the engineered nuclease comprises a replacement or substitution at positions 160, 542, and 842. In some embodiments, the engineered nuclease comprises a replacement or substitution at positions 160, 547, and 553. In some embodiments, the engineered nuclease comprises a replacement or substitution at positions 160, 547, and 842. In some embodiments, the engineered nuclease comprises a replacement or substitution at positions 160, 553, and 842. In some embodiments, the engineered nuclease comprises a replacement or substitution at positions 265, 542, and 547. In some embodiments, the engineered nuclease comprises a replacement or substitution at positions 265, 542, and 553. In some embodiments, the engineered nuclease comprises a replacement or substitution at positions 265, 542, and 842. In some embodiments, the engineered nuclease comprises a replacement or substitution at positions 265, 547, and 553. In some embodiments, the engineered nuclease comprises a replacement or substitution at positions 265, 547, and 842. In some embodiments, the engineered nuclease comprises a replacement or substitution at positions 265, 553, and 842. In some embodiments, the engineered nuclease comprises a replacement or substitution at positions 542, 547, and 553. In some embodiments, the engineered nuclease comprises a replacement or substitution at positions 542, 547, and 842. In some embodiments, the engineered nuclease comprises a replacement or substitution at positions 542, 553, and 842. In some embodiments, the engineered nuclease comprises a replacement or substitution at positions 547, 553 and 842.

In some embodiments, the amino acid sequence lacks one or more of the following features: E at position 160, N at position 265, N at position 542, D at position 547, K at position 553, and E at position 842. In some embodiments, the replacement at position 160 is an E160R replacement. In some embodiments, the replacement at position 265 is a N265A replacement. In some embodiments, the replacement at position 542 is an N542R replacement. In some embodiments, the replacement at 547 is a D547R replacement. In some embodiments, the replacement at 553 is a K553R replacement. In some embodiments, the replacement at 842 is a E842L replacement.

In some embodiments, the one or more amino acid replacements is selected from the group consisting of E160R, N265A, N542R, D547R, K553R, and E842L, in reference to SEQ ID NO: 7. In some embodiments, the one or more amino acid replacements comprises an E160R replacement, and N265A replacement, an N542R replacement, an D547R replacement, an K553R replacement, or an E842L replacement, in reference to SEQ ID NO: 7.

In some embodiments, an engineered nuclease comprises an amino acid sequence with at least 90% (e.g., at least 90%, at least 91%, at least 92%, at least 93%, at least 94%, at least 95%, at least 96%, at least 97%, at least 98%, at least 99%) to SEQ ID NO: 9 with one or more amino acid replacements corresponding to positions selected from the group consisting of 161, 244, 516, 521, and 527. In some embodiments, the engineered nuclease comprises a replacement or substitution at position 161. In some embodiments, the engineered nuclease comprises a replacement or substitution at position 244. In some embodiments, the engineered nuclease comprises a replacement or substitution at position 516. In some embodiments, the engineered nuclease comprises a replacement or substitution at position 521. In some embodiments, the engineered nuclease comprises a replacement or substitution at position 527.

In some embodiments, the engineered nuclease comprises two amino acid replacements at positions selected from the group consisting of 161, 244, 516, 521, and 527, in reference to SEQ ID NO: 9. In some embodiments, the engineered nuclease comprises a replacement or substitution at positions 161 and 244. In some embodiments, the engineered nuclease comprises a replacement or substitution at positions 161 and 516. In some embodiments, the engineered nuclease comprises a replacement or substitution at positions 161 and 521. In some embodiments, the engineered nuclease comprises a replacement or substitution at positions 161 and 527. In some embodiments, the engineered nuclease comprises a replacement or substitution at positions 244 and 516. In some embodiments, the engineered nuclease comprises a replacement or substitution at positions 244 and 521. In some embodiments, the engineered nuclease comprises a replacement or substitution at positions 244 and 527. In some embodiments, the engineered nuclease comprises a replacement or substitution at positions 516 and 521. In some embodiments, the engineered nuclease comprises a replacement or substitution at positions 516 and 527. In some embodiments, the engineered nuclease comprises a replacement or substitution at positions 521 and 527.

In some embodiments, the engineered nuclease comprises three amino acid replacements at positions selected from the group consisting of 161, 244, 516, 521, and 527, in reference to SEQ ID NO: 9. In some embodiments, the engineered nuclease comprises a replacement or substitution at positions 161, 244, and 516. In some embodiments, the engineered nuclease comprises a replacement or substitution at positions 161, 244, and 521. In some embodiments, the engineered nuclease comprises a replacement or substitution at positions 161, 244, and 527. In some embodiments, the engineered nuclease comprises a replacement or substitution at positions 161, 516, and 521. In some embodiments, the engineered nuclease comprises a replacement or substitution at positions 161, 516, and 527. In some embodiments, the engineered nuclease comprises a replacement or substitution at positions 161, 521, and 527. In some embodiments, the engineered nuclease comprises a replacement or substitution at positions 244, 516, and 521. In some embodiments, the engineered nuclease comprises a replacement or substitution at positions 244, 516, and 527. In some embodiments, the engineered nuclease comprises a replacement or substitution at positions 244, 521, and 527. In some embodiments, the engineered nuclease comprises a replacement or substitution at positions 516, 521, and 527.

In some embodiments, the amino acid sequence lacks one or more of the following features: E at position 161, N at position 244, S at position 516, G at position 521, and K at position 527. In some embodiments, the replacement at position 161 is an E161R replacement. In some embodiments, the replacement at position 244 is a N244A replacement. In some embodiments, the replacement at position 516 is an S516R replacement. In some embodiments, the replacement at 521 is a G521R replacement. In some embodiments, the replacement at 527 is a K527R replacement. In some embodiments, the one or more amino acid replacements is selected from the group consisting of E161R, N244A, S516R, G521R, and K527R, in reference to SEQ ID NO: 9. In some embodiments, the one or more amino acid replacements comprises an E161R replacement, an N244A replacement, an S516R replacement, a G521R replacement, or a K527R replacement, in reference to SEQ ID NO: 9.

In some embodiments, an engineered nuclease comprises an amino acid sequence with at least 90% (e.g., at least 90%, at least 91%, at least 92%, at least 93%, at least 94%, at least 95%, at least 96%, at least 97%, at least 98%, at least 99%) to SEQ ID NO: 10 with one or more amino acid replacements corresponding to positions selected from the group consisting of 151, 255, 523, 528, and 534. In some embodiments, the engineered nuclease comprises a replacement or substitution at position 151. In some embodiments, the engineered nuclease comprises a replacement or substitution at position 255. In some embodiments, the engineered nuclease comprises a replacement or substitution at position 523. In some embodiments, the engineered nuclease comprises a replacement or substitution at position 528. In some embodiments, the engineered nuclease comprises a replacement or substitution at position 534.

In some embodiments, the engineered nuclease comprises two amino acid replacements at positions selected from the group consisting of 151, 255, 523, 528, and 534, in reference to SEQ ID NO: 10. In some embodiments, the engineered nuclease comprises a replacement or substitution at positions 151 and 255. In some embodiments, the engineered nuclease comprises a replacement or substitution at positions 151 and 523. In some embodiments, the engineered nuclease comprises a replacement or substitution at positions 151 and 528. In some embodiments, the engineered nuclease comprises a replacement or substitution at positions 151 and 534. In some embodiments, the engineered nuclease comprises a replacement or substitution at positions 255 and 523. In some embodiments, the engineered nuclease comprises a replacement or substitution at positions 255 and 528. In some embodiments, the engineered nuclease comprises a replacement or substitution at positions 255 and 534. In some embodiments, the engineered nuclease comprises a replacement or substitution at positions 523 and 528. In some embodiments, the engineered nuclease comprises a replacement or substitution at positions 523 and 534. In some embodiments, the engineered nuclease comprises a replacement or substitution at positions 528 and 534.

In some embodiments, the engineered nuclease comprises three amino acid replacements at positions selected from the group consisting of 151, 255, 523, 528, and 534, in reference to SEQ ID NO: 10. In some embodiments, the engineered nuclease comprises a replacement or substitution at positions 151, 255, and 523. In some embodiments, the engineered nuclease comprises a replacement or substitution at positions 151, 255, and 528. In some embodiments, the engineered nuclease comprises a replacement or substitution at positions 151, 255, and 534. In some embodiments, the engineered nuclease comprises a replacement or substitution at positions 151, 523, and 528. In some embodiments, the engineered nuclease comprises a replacement or substitution at positions 151, 523, and 534. In some embodiments, the engineered nuclease comprises a replacement or substitution at positions 151, 528, and 534. In some embodiments, the engineered nuclease comprises a replacement or substitution at positions 255, 523, and 528. In some embodiments, the engineered nuclease comprises a replacement or substitution at positions 255, 523, and 534. In some embodiments, the engineered nuclease comprises a replacement or substitution at positions 255, 528, and 534. In some embodiments, the engineered nuclease comprises a replacement or substitution at positions 523, 528, and 534.

In some embodiments, the amino acid sequence lacks one or more of the following features: N at position 151, N at position 255, N at position 523, G at position 528, and K at position 534. In some embodiments, the replacement at position 151 is an N151R replacement. In some embodiments, the replacement at position 255 is a N255A replacement. In some embodiments, the replacement at position 523 is an N523R replacement. In some embodiments, the replacement at 528 is a G528R replacement. In some embodiments, the replacement at 534 is a K534R replacement. In some embodiments, the one or more amino acid replacements is selected from the group consisting of NISIR, N255A, N523R, G528R, and K534R, in reference to SEQ ID NO: 10. In some embodiments, the one or more amino acid replacements comprises an N151R replacement, an N255A replacement, an N523R replacement, a G528R replacement, or a K534R replacement, in reference to SEQ ID NO: 10.

In some embodiments, an engineered nuclease comprises an amino acid sequence with at least 90% (e.g., at least 90%, at least 91%, at least 92%, at least 93%, at least 94%, at least 95%, at least 96%, at least 97%, at least 98%, at least 99%) to SEQ ID NO: 11 with one or more amino acid replacements corresponding to positions selected from the group consisting of 187, 267, 547, 552, and 558. In some embodiments, the engineered nuclease comprises a replacement or substitution at position 187. In some embodiments, the engineered nuclease comprises a replacement or substitution at position 267. In some embodiments, the engineered nuclease comprises a replacement or substitution at position 547. In some embodiments, the engineered nuclease comprises a replacement or substitution at position 552. In some embodiments, the engineered nuclease comprises a replacement or substitution at position 558.

In some embodiments, the engineered nuclease comprises two amino acid replacements at positions selected from the group consisting of 187, 267, 547, 552, and 558, in reference to SEQ ID NO: 11. In some embodiments, the engineered nuclease comprises a replacement or substitution at positions 187 and 267. In some embodiments, the engineered nuclease comprises a replacement or substitution at positions 187 and 547. In some embodiments, the engineered nuclease comprises a replacement or substitution at positions 187 and 552. In some embodiments, the engineered nuclease comprises a replacement or substitution at positions 187 and 558. In some embodiments, the engineered nuclease comprises a replacement or substitution at positions 267 and 547. In some embodiments, the engineered nuclease comprises a replacement or substitution at positions 267 and 552. In some embodiments, the engineered nuclease comprises a replacement or substitution at positions 267 and 558. In some embodiments, the engineered nuclease comprises a replacement or substitution at positions 547 and 552. In some embodiments, the engineered nuclease comprises a replacement or substitution at positions 547 and 558. In some embodiments, the engineered nuclease comprises a replacement or substitution at positions 552 and 558.

In some embodiments, the engineered nuclease comprises three amino acid replacements at positions selected from the group consisting of 187, 267, 547, 552, and 558, in reference to SEQ ID NO: 11. In some embodiments, the engineered nuclease comprises a replacement or substitution at positions 187, 267, and 547. In some embodiments, the engineered nuclease comprises a replacement or substitution at positions 187, 267, and 552. In some embodiments, the engineered nuclease comprises a replacement or substitution at positions 187, 267, and 558. In some embodiments, the engineered nuclease comprises a replacement or substitution at positions 187, 547, and 552. In some embodiments, the engineered nuclease comprises a replacement or substitution at positions 187, 547, and 558. In some embodiments, the engineered nuclease comprises a replacement or substitution at positions 187, 552, and 558. In some embodiments, the engineered nuclease comprises a replacement or substitution at positions 267, 547, and 552. In some embodiments, the engineered nuclease comprises a replacement or substitution at positions 267, 547, and 558. In some embodiments, the engineered nuclease comprises a replacement or substitution at positions 267, 552, and 558. In some embodiments, the engineered nuclease comprises a replacement or substitution at positions 547, 552, and 558.

In some embodiments, the amino acid sequence lacks one or more of the following features: Q at position 187, N at position 267, K at position 547, M at position 552, and E at position 558. In some embodiments, the replacement at position 187 is an Q187R replacement. In some embodiments, the replacement at position 267 is a N267A replacement. In some embodiments, the replacement at position 547 is an K547R replacement. In some embodiments, the replacement at 552 is a M552R replacement. In some embodiments, the replacement at 558 is a E558R replacement. In some embodiments, the one or more amino acid replacements is selected from the group consisting of Q187R, N267A, K547R, M552R, and E558R, in reference to SEQ ID NO: 11. In some embodiments, the one or more amino acid replacements comprises an Q187R replacement, an N267A replacement, an K547R replacement, a M552R replacement, or a E558R replacement, in reference to SEQ ID NO: 11.

In some embodiments, an engineered nuclease comprises an amino acid sequence with at least 90% (e.g., at least 90%, at least 91%, at least 92%, at least 93%, at least 94%, at least 95%, at least 96%, at least 97%, at least 98%, at least 99%) to SEQ ID NO: 12 with one or more amino acid replacements corresponding to positions selected from the group consisting of 158, 263, 531, 526, and 537. In some embodiments, the engineered nuclease comprises a replacement or substitution at position 158. In some embodiments, the engineered nuclease comprises a replacement or substitution at position 263. In some embodiments, the engineered nuclease comprises a replacement or substitution at position 531. In some embodiments, the engineered nuclease comprises a replacement or substitution at position 526. In some embodiments, the engineered nuclease comprises a replacement or substitution at position 537.

In some embodiments, the engineered nuclease comprises two amino acid replacements at positions selected from the group consisting of 158, 263, 547, 526, and 537, in reference to SEQ ID NO: 12. In some embodiments, the engineered nuclease comprises a replacement or substitution at positions 158 and 263. In some embodiments, the engineered nuclease comprises a replacement or substitution at positions 158 and 547. In some embodiments, the engineered nuclease comprises a replacement or substitution at positions 158 and 526. In some embodiments, the engineered nuclease comprises a replacement or substitution at positions 158 and 537. In some embodiments, the engineered nuclease comprises a replacement or substitution at positions 263 and 547. In some embodiments, the engineered nuclease comprises a replacement or substitution at positions 263 and 526. In some embodiments, the engineered nuclease comprises a replacement or substitution at positions 263 and 537. In some embodiments, the engineered nuclease comprises a replacement or substitution at positions 547 and 526. In some embodiments, the engineered nuclease comprises a replacement or substitution at positions 547 and 537. In some embodiments, the engineered nuclease comprises a replacement or substitution at positions 526 and 537.

In some embodiments, the engineered nuclease comprises three amino acid replacements at positions selected from the group consisting of 158, 263, 547, 526, and 537, in reference to SEQ ID NO: 12. In some embodiments, the engineered nuclease comprises a replacement or substitution at positions 158, 263, and 547. In some embodiments, the engineered nuclease comprises a replacement or substitution at positions 158, 263, and 526. In some embodiments, the engineered nuclease comprises a replacement or substitution at positions 158, 263, and 537. In some embodiments, the engineered nuclease comprises a replacement or substitution at positions 158, 547, and 526. In some embodiments, the engineered nuclease comprises a replacement or substitution at positions 158, 547, and 537. In some embodiments, the engineered nuclease comprises a replacement or substitution at positions 158, 526, and 537. In some embodiments, the engineered nuclease comprises a replacement or substitution at positions 263, 547, and 526. In some embodiments, the engineered nuclease comprises a replacement or substitution at positions 263, 547, and 537 In some embodiments, the engineered nuclease comprises a replacement or substitution at positions 263, 526, and 537. In some embodiments, the engineered nuclease comprises a replacement or substitution at positions 547, 526, and 537.

In some embodiments, the amino acid sequence lacks one or more of the following features: K at position 158, N at position 263, N at position 547, C at position 526, and K at position 537. In some embodiments, the replacement at position 158 is an K158R replacement. In some embodiments, the replacement at position 263 is a N263A replacement. In some embodiments, the replacement at position 547 is an N547R replacement. In some embodiments, the replacement at 526 is a C526R replacement. In some embodiments, the replacement at 537 is a K537R replacement. In some embodiments, the one or more amino acid replacements is selected from the group consisting of K158R, N263A, N547R, C526R, and K537R, in reference to SEQ ID NO: 12. In some embodiments, the one or more amino acid replacements comprises an K158R replacement, an N263A replacement, an N547R replacement, a C526R replacement, or a K537R replacement, in reference to SEQ ID NO: 12.

In some embodiments, an engineered nuclease comprises an amino acid sequence with at least 90% (e.g., at least 90%, at least 91%, at least 92%, at least 93%, at least 94%, at least 95%, at least 96%, at least 97%, at least 98%, at least 99%) to SEQ ID NO: 13 with one or more amino acid replacements corresponding to positions selected from the group consisting of 158, 263, 531, 526, and 537. In some embodiments, the engineered nuclease comprises a replacement or substitution at position 158. In some embodiments, the engineered nuclease comprises a replacement or substitution at position 263. In some embodiments, the engineered nuclease comprises a replacement or substitution at position 531. In some embodiments, the engineered nuclease comprises a replacement or substitution at position 526. In some embodiments, the engineered nuclease comprises a replacement or substitution at position 537.

In some embodiments, the engineered nuclease comprises two amino acid replacements at positions selected from the group consisting of 158, 263, 531, 526, and 537, in reference to SEQ ID NO: 13. In some embodiments, the engineered nuclease comprises a replacement or substitution at positions 158 and 263. In some embodiments, the engineered nuclease comprises a replacement or substitution at positions 158 and 531. In some embodiments, the engineered nuclease comprises a replacement or substitution at positions 158 and 526. In some embodiments, the engineered nuclease comprises a replacement or substitution at positions 158 and 537. In some embodiments, the engineered nuclease comprises a replacement or substitution at positions 263 and 531. In some embodiments, the engineered nuclease comprises a replacement or substitution at positions 263 and 526. In some embodiments, the engineered nuclease comprises a replacement or substitution at positions 263 and 537. In some embodiments, the engineered nuclease comprises a replacement or substitution at positions 531 and 526. In some embodiments, the engineered nuclease comprises a replacement or substitution at positions 531 and 537. In some embodiments, the engineered nuclease comprises a replacement or substitution at positions 526 and 537.

In some embodiments, the engineered nunclease comprises three amino acid replacements at positions selected from the group consisting of 158, 263, 531, 526, and 537, in reference to SEQ ID NO: 13. In some embodiments, the engineered nuclease comprises a replacement or substitution at positions 158, 263, and 531. In some embodiments, the engineered nuclease comprises a replacement or substitution at positions 158, 263, and 526. In some embodiments, the engineered nuclease comprises a replacement or substitution at positions 158, 263, and 537. In some embodiments, the engineered nuclease comprises a replacement or substitution at positions 158, 531, and 526. In some embodiments, the engineered nuclease comprises a replacement or substitution at positions 158, 531, and 537. In some embodiments, the engineered nuclease comprises a replacement or substitution at positions 158, 526, and 537. In some embodiments, the engineered nuclease comprises a replacement or substitution at positions 263, 531, and 526. In some embodiments, the engineered nuclease comprises a replacement or substitution at positions 263, 531, and 537. In some embodiments, the engineered nuclease comprises a replacement or substitution at positions 263, 526, and 537. In some embodiments, the engineered nuclease comprises a replacement or substitution at positions 531, 526, and 537.

In some embodiments, the amino acid sequence lacks one or more of the following features: K at position 158, N at position 263, N at position 531, C at position 526, and K at position 537. In some embodiments, the replacement at position 158 is an K158R replacement. In some embodiments, the replacement at position 263 is a N263A replacement. In some embodiments, the replacement at position 531 is an N531R replacement. In some embodiments, the replacement at 526 is a C526R replacement. In some embodiments, the replacement at 537 is a K537R replacement. In some embodiments, the one or more amino acid replacements is selected from the group consisting of K158R, N263A, N531R, C526R, and K537R, in reference to SEQ ID NO: 13. In some embodiments, the one or more amino acid replacements comprises an K158R replacement, an N263A replacement, an N531R replacement, a C526R replacement, or a K537R replacement, in reference to SEQ ID NO: 13.

In some embodiments, an engineered nuclease comprises an amino acid sequence with at least 90% (e.g., at least 90%, at least 91%, at least 92%, at least 93%, at least 94%, at least 95%, at least 96%, at least 97%, at least 98%, at least 99%) to SEQ ID NO: 14 with one or more amino acid replacements corresponding to positions selected from the group consisting of 159, 259, 532, 537, 543 and 801. In some embodiments, the engineered nuclease comprises a replacement or substitution at position 159. In some embodiments, the engineered nuclease comprises a replacement or substitution at position 259. In some embodiments, the engineered nuclease comprises a replacement or substitution at position 532. In some embodiments, the engineered nuclease comprises a replacement or substitution at position 537. In some embodiments, the engineered nuclease comprises a replacement or substitution at position 542. In some embodiments, the engineered nuclease comprises a replacement or substitution at position 801.

In some embodiments, the engineered nuclease comprises two amino acid replacements at positions selected from the group consisting of 159, 259, 532, 537, 543 and 801, in reference to SEQ ID NO: 14. In some embodiments, the engineered nuclease comprises a replacement or substitution at positions 159 and 259. In some embodiments, the engineered nuclease comprises a replacement or substitution at positions 159 and 532. In some embodiments, the engineered nuclease comprises a replacement or substitution at positions 159 and 537. In some embodiments, the engineered nuclease comprises a replacement or substitution at positions 159 and 543. In some embodiments, the engineered nuclease comprises a replacement or substitution at positions 159 and 801. In some embodiments, the engineered nuclease comprises a replacement or substitution at positions 259 and 532. In some embodiments, the engineered nuclease comprises a replacement or substitution at positions 259 and 537. In some embodiments, the engineered nuclease comprises a replacement or substitution at positions 259 and 543. In some embodiments, the engineered nuclease comprises a replacement or substitution at positions 259 and 801. In some embodiments, the engineered nuclease comprises a replacement or substitution at positions 532 and 537. In some embodiments, the engineered nuclease comprises a replacement or substitution at positions 532 and 543. In some embodiments, the engineered nuclease comprises a replacement or substitution at positions 532 and 801. In some embodiments, the engineered nuclease comprises a replacement or substitution at positions 537 and 543. In some embodiments, the engineered nuclease comprises a replacement or substitution at positions 537 and 801. In some embodiments, the engineered nuclease comprises a replacement or substitution at positions 543 and 801.

In some embodiments, the engineered nuclease comprises three amino acid replacements at positions selected from the group consisting of 159, 259, 532, 537, 543 and 801, in reference to SEQ ID NO: 14. In some embodiments, the engineered nuclease comprises a replacement or substitution at positions 159, 259, and 532. In some embodiments, the engineered nuclease comprises a replacement or substitution at positions 159, 259, and 537. In some embodiments, the engineered nuclease comprises a replacement or substitution at positions 159, 259, and 543. In some embodiments, the engineered nuclease comprises a replacement or substitution at positions 159, 259, and 801. In some embodiments, the engineered nuclease comprises a replacement or substitution at positions 159, 532, and 537. In some embodiments, the engineered nuclease comprises a replacement or substitution at positions 159, 532, and 543. In some embodiments, the engineered nuclease comprises a replacement or substitution at positions 159, 532, and 801. In some embodiments, the engineered nuclease comprises a replacement or substitution at positions 159, 537, and 543. In some embodiments, the engineered nuclease comprises a replacement or substitution at positions 159, 537, and 801. In some embodiments, the engineered nuclease comprises a replacement or substitution at positions 159, 543, and 801. In some embodiments, the engineered nuclease comprises a replacement or substitution at positions 259, 532, and 537. In some embodiments, the engineered nuclease comprises a replacement or substitution at positions 259, 532, and 543. In some embodiments, the engineered nuclease comprises a replacement or substitution at positions 259, 532, and 801. In some embodiments, the engineered nuclease comprises a replacement or substitution at positions 259, 537, and 543. In some embodiments, the engineered nuclease comprises a replacement or substitution at positions 259, 537, and 801. In some embodiments, the engineered nuclease comprises a replacement or substitution at positions 259, 543, and 801. In some embodiments, the engineered nuclease comprises a replacement or substitution at positions 532, 537, and 543. In some embodiments, the engineered nuclease comprises a replacement or substitution at positions 532, 537, and 801. In some embodiments, the engineered nuclease comprises a replacement or substitution at positions 532, 543, and 801. In some embodiments, the engineered nuclease comprises a replacement or substitution at positions 537, 543 and 801.

In some embodiments, the amino acid sequence lacks one or more of the following features: K at position 159, N at position 259, N at position 532, N at position 537, K at position 543, and Q at position 801. In some embodiments, the replacement at position 159 is an K159R replacement. In some embodiments, the replacement at position 259 is a N259A replacement. In some embodiments, the replacement at position 532 is an N532R replacement. In some embodiments, the replacement at 537 is a N537R replacement. In some embodiments, the replacement at 543 is a K543R replacement. In some embodiments, the replacement at 801 is a Q801L replacement.

In some embodiments, the one or more amino acid replacements is selected from the group consisting of K159R, N259A, N532R, N537R, K543R and Q801L, in reference to SEQ ID NO: 14. In some embodiments, the one or more amino acid replacements comprises an K159R replacement, and N259A replacement, an N532R replacement, an N537R replacement, an K543R replacement, or an Q801L replacement, in reference to SEQ ID NO: 14.

In some embodiments, an engineered nuclease comprises an amino acid sequence with at least 90% or (e.g., at least 90%, at least 91%, at least 92%, at least 93%, at least 94%, at least 95%, at least 96%, at least 97%, at least 98%, at least 99%) to SEQ ID NO: 15 with one or more amino acid replacements corresponding to positions selected from the group consisting of 158, 264, 525, 530, and 536. In some embodiments, the engineered nuclease comprises a replacement or substitution at position 158. In some embodiments, the engineered nuclease comprises a replacement or substitution at position 264. In some embodiments, the engineered nuclease comprises a replacement or substitution at position 525. In some embodiments, the engineered nuclease comprises a replacement or substitution at position 530. In some embodiments, the engineered nuclease comprises a replacement or substitution at position 536.

In some embodiments, the engineered nuclease comprises two amino acid replacements at positions selected from the group consisting of 158, 264, 525, 530, and 536 in reference to SEQ ID NO: 15. In some embodiments, the engineered nuclease comprises a replacement or substitution at positions 158 and 264. In some embodiments, the engineered nuclease comprises a replacement or substitution at positions 158 and 525. In some embodiments, the engineered nuclease comprises a replacement or substitution at positions 158 and 530. In some embodiments, the engineered nuclease comprises a replacement or substitution at positions 158 and 536. In some embodiments, the engineered nuclease comprises a replacement or substitution at positions 264 and 525. In some embodiments, the engineered nuclease comprises a replacement or substitution at positions 264 and 530. In some embodiments, the engineered nuclease comprises a replacement or substitution at positions 264 and 536. In some embodiments, the engineered nuclease comprises a replacement or substitution at positions 525 and 530. In some embodiments, the engineered nuclease comprises a replacement or substitution at positions 525 and 536. In some embodiments, the engineered nuclease comprises a replacement or substitution at positions 530 and 536.

In some embodiments, the engineered nuclease comprises three amino acid replacements at positions selected from the group consisting of 158, 264, 525, 530, and 536, in reference to SEQ ID NO: 15. In some embodiments, the engineered nuclease comprises a replacement or substitution at positions 158, 264, and 525. In some embodiments, the engineered nuclease comprises a replacement or substitution at positions 158, 264, and 530. In some embodiments, the engineered nuclease comprises a replacement or substitution at positions 158, 264, and 536. In some embodiments, the engineered nuclease comprises a replacement or substitution at positions 158, 525, and 530. In some embodiments, the engineered nuclease comprises a replacement or substitution at positions 158, 525, and 536. In some embodiments, the engineered nuclease comprises a replacement or substitution at positions 158, 530, and 536. In some embodiments, the engineered nuclease comprises a replacement or substitution at positions 264, 525, and 530. In some embodiments, the engineered nuclease comprises a replacement or substitution at positions 264, 525, and 536. In some embodiments, the engineered nuclease comprises a replacement or substitution at positions 264, 530, and 536. In some embodiments, the engineered nuclease comprises a replacement or substitution at positions 525, 530, and 536.

In some embodiments, the amino acid sequence lacks one or more of the following features: E at position 158, N at position 264, N at position 525, N at position 530, and K at position 536. In some embodiments, the replacement at position 158 is an E158R replacement. In some embodiments, the replacement at position 264 is a N264A replacement. In some embodiments, the replacement at position 525 is an N525R replacement. In some embodiments, the replacement at 530 is a N530R replacement. In some embodiments, the replacement at 536 is a K536R replacement. In some embodiments, the one or more amino acid replacements is selected from the group consisting of E158R, N264A, N525R, N530R, and K536R, in reference to SEQ ID NO: 15. In some embodiments, the one or more amino acid replacements comprises an E158R replacement, an N264A replacement, an N525R replacement, a N530R replacement, or a K536R replacement, in reference to SEQ ID NO: 15.

In some embodiments, an engineered nuclease comprises an amino acid sequence with at least 90% (e.g., at least 90%, at least 91%, at least 92%, at least 93%, at least 94%, at least 95%, at least 96%, at least 97%, at least 98%, at least 99%) to SEQ ID NO: 16 with one or more amino acid replacements corresponding to positions selected from the group consisting of 175, 256, 542, 548, and 537. In some embodiments, the engineered nuclease comprises a replacement or substitution at position 175. In some embodiments, the engineered nuclease comprises a replacement or substitution at position 256. In some embodiments, the engineered nuclease comprises a replacement or substitution at position 542. In some embodiments, the engineered nuclease comprises a replacement or substitution at position 548. In some embodiments, the engineered nuclease comprises a replacement or substitution at position 537.

In some embodiments, the engineered nuclease comprises two amino acid replacements at positions selected from the group consisting of 175, 256, 542, 548, and 537 in reference to SEQ ID NO: 16. In some embodiments, the engineered nuclease comprises a replacement or substitution at positions 175 and 256. In some embodiments, the engineered nuclease comprises a replacement or substitution at positions 175 and 542. In some embodiments, the engineered nuclease comprises a replacement or substitution at positions 175 and 548. In some embodiments, the engineered nuclease comprises a replacement or substitution at positions 175 and 537. In some embodiments, the engineered nuclease comprises a replacement or substitution at positions 256 and 542. In some embodiments, the engineered nuclease comprises a replacement or substitution at positions 256 and 548. In some embodiments, the engineered nuclease comprises a replacement or substitution at positions 256 and 537. In some embodiments, the engineered nuclease comprises a replacement or substitution at positions 542 and 548. In some embodiments, the engineered nuclease comprises a replacement or substitution at positions 542 and 537. In some embodiments, the engineered nuclease comprises a replacement or substitution at positions 548 and 537.

In some embodiments, the engineered nuclease comprises three amino acid replacements at positions selected from the group consisting of 175, 256, 542, 548, and 537, in reference to SEQ ID NO: 16. In some embodiments, the engineered nuclease comprises a replacement or substitution at positions 175, 256, and 542. In some embodiments, the engineered nuclease comprises a replacement or substitution at positions 175, 256, and 548. In some embodiments, the engineered nuclease comprises a replacement or substitution at positions 175, 256, and 537. In some embodiments, the engineered nuclease comprises a replacement or substitution at positions 175, 542, and 548. In some embodiments, the engineered nuclease comprises a replacement or substitution at positions 175, 542, and 537. In some embodiments, the engineered nuclease comprises a replacement or substitution at positions 175, 548, and 537. In some embodiments, the engineered nuclease comprises a replacement or substitution at positions 256, 542, and 548. In some embodiments, the engineered nuclease comprises a replacement or substitution at positions 256, 542, and 537. In some embodiments, the engineered nuclease comprises a replacement or substitution at positions 256, 548, and 537. In some embodiments, the engineered nuclease comprises a replacement or substitution at positions 542, 548, and 537.

In some embodiments, the amino acid sequence lacks one or more of the following features: K at position 175, N at position 256, C at position 537, T at position 542, and K at position 548. In some embodiments, the replacement at position 175 is an K175R replacement. In some embodiments, the replacement at position 256 is a N256A replacement. In some embodiments, the replacement at position 537 is an C537R replacement. In some embodiments, the replacement at 542 is a T542R replacement. In some embodiments, the replacement at 548 is a K548R replacement. In some embodiments, the one or more amino acid replacements is selected from the group consisting of K175R, N256A, C537R, T542R and K548R, in reference to SEQ ID NO: 16. In some embodiments, the one or more amino acid replacements comprises an K175R replacement, an N256A replacement, an C537R replacement, a C537R replacement, or a K548R replacement, in reference to SEQ ID NO: 16.

In some embodiments, an engineered nuclease comprises an amino acid sequence with at least 90% (e.g., at least 90%, at least 91%, at least 92%, at least 93%, at least 94%, at least 95%, at least 96%, at least 97%, at least 98%, at least 99%) to SEQ ID NO: 17 with one or more amino acid replacements corresponding to positions selected from the group consisting of 160, 265, 527, 532, and 538. In some embodiments, the engineered nuclease comprises a replacement or substitution at position 160. In some embodiments, the engineered nuclease comprises a replacement or substitution at position 265. In some embodiments, the engineered nuclease comprises a replacement or substitution at position 527. In some embodiments, the engineered nuclease comprises a replacement or substitution at position 532. In some embodiments, the engineered nuclease comprises a replacement or substitution at position 538.

In some embodiments, the engineered nuclease comprises two amino acid replacements at positions selected from the group consisting of 160, 265, 527, 532, and 538, in reference to SEQ ID NO: 17. In some embodiments, the engineered nuclease comprises a replacement or substitution at positions 160 and 265. In some embodiments, the engineered nuclease comprises a replacement or substitution at positions 160 and 527. In some embodiments, the engineered nuclease comprises a replacement or substitution at positions 160 and 532. In some embodiments, the engineered nuclease comprises a replacement or substitution at positions 160 and 538. In some embodiments, the engineered nuclease comprises a replacement or substitution at positions 265 and 527. In some embodiments, the engineered nuclease comprises a replacement or substitution at positions 265 and 532. In some embodiments, the engineered nuclease comprises a replacement or substitution at positions 265 and 538. In some embodiments, the engineered nuclease comprises a replacement or substitution at positions 527 and 532. In some embodiments, the engineered nuclease comprises a replacement or substitution at positions 527 and 538. In some embodiments, the engineered nuclease comprises a replacement or substitution at positions 532 and 538.

In some embodiments, the engineered nuclease comprises three amino acid replacements at positions selected from the group consisting of 160, 265, 527, 532, and 538, in reference to SEQ ID NO: 17. In some embodiments, the engineered nuclease comprises a replacement or substitution at positions 160, 265, and 527. In some embodiments, the engineered nuclease comprises a replacement or substitution at positions 160, 265, and 532. In some embodiments, the engineered nuclease comprises a replacement or substitution at positions 160, 265, and 538. In some embodiments, the engineered nuclease comprises a replacement or substitution at positions 160, 527, and 532. In some embodiments, the engineered nuclease comprises a replacement or substitution at positions 160, 527, and 538. In some embodiments, the engineered nuclease comprises a replacement or substitution at positions 160, 532, and 538. In some embodiments, the engineered nuclease comprises a replacement or substitution at positions 265, 527, and 532. In some embodiments, the engineered nuclease comprises a replacement or substitution at positions 265, 527, and 538. In some embodiments, the engineered nuclease comprises a replacement or substitution at positions 265, 532, and 538. In some embodiments, the engineered nuclease comprises a replacement or substitution at positions 527, 532, and 538.

In some embodiments, the amino acid sequence lacks one or more of the following features: K at position 160, N at position 265, C at position 527, S at position 532, and K at position 538. In some embodiments, the replacement at position 160 is an K160R replacement. In some embodiments, the replacement at position 265 is a N265A replacement. In some embodiments, the replacement at position 537 is an C527R replacement. In some embodiments, the replacement at 532 is a S532R replacement. In some embodiments, the replacement at 538 is a K538R replacement. In some embodiments, the one or more amino acid replacements is selected from the group consisting of K160R, N265A, C527R, S532R, and K538R, in reference to SEQ ID NO: 17. In some embodiments, the one or more amino acid replacements comprises an K160R replacement, an N265A replacement, an C527R replacement, a S532R replacement, or a K538R replacement, in reference to SEQ ID NO: 17.

In some embodiments, an engineered nuclease comprises an amino acid sequence with at least 90% (e.g., at least 90%, at least 91%, at least 92%, at least 93%, at least 94%, at least 95%, at least 96%, at least 97%, at least 98%, at least 99%) to SEQ ID NO: 18 with one or more amino acid replacements corresponding to positions selected from the group consisting of 152, 256, 515, 520, 526, and 775. In some embodiments, the engineered nuclease comprises a replacement or substitution at position 152. In some embodiments, the engineered nuclease comprises a replacement or substitution at position 256. In some embodiments, the engineered nuclease comprises a replacement or substitution at position 515. In some embodiments, the engineered nuclease comprises a replacement or substitution at position 520. In some embodiments, the engineered nuclease comprises a replacement or substitution at position 526. In some embodiments, the engineered nuclease comprises a replacement or substitution at position 775.

In some embodiments, the engineered nuclease comprises two amino acid replacements at positions selected from the group consisting of 152, 256, 515, 520, 526, and 775, in reference to SEQ ID NO: 18. In some embodiments, the engineered nuclease comprises a replacement or substitution at positions 152 and 256. In some embodiments, the engineered nuclease comprises a replacement or substitution at positions 152 and 515. In some embodiments, the engineered nuclease comprises a replacement or substitution at positions 152 and 520. In some embodiments, the engineered nuclease comprises a replacement or substitution at positions 152 and 526. In some embodiments, the engineered nuclease comprises a replacement or substitution at positions 152 and 775. In some embodiments, the engineered nuclease comprises a replacement or substitution at positions 256 and 515. In some embodiments, the engineered nuclease comprises a replacement or substitution at positions 256 and 520. In some embodiments, the engineered nuclease comprises a replacement or substitution at positions 256 and 526. In some embodiments, the engineered nuclease comprises a replacement or substitution at positions 256 and 775. In some embodiments, the engineered nuclease comprises a replacement or substitution at positions 515 and 520. In some embodiments, the engineered nuclease comprises a replacement or substitution at positions 515 and 526. In some embodiments, the engineered nuclease comprises a replacement or substitution at positions 515 and 775. In some embodiments, the engineered nuclease comprises a replacement or substitution at positions 520 and 526. In some embodiments, the engineered nuclease comprises a replacement or substitution at positions 520 and 775. In some embodiments, the engineered nuclease comprises a replacement or substitution at positions 526 and 775.

In some embodiments, the engineered nuclease comprises three amino acid replacements at positions selected from the group consisting of 152, 256, 515, 520, 526, and 775, in reference to SEQ ID NO: 18. In some embodiments, the engineered nuclease comprises a replacement or substitution at positions 152, 256, and 515. In some embodiments, the engineered nuclease comprises a replacement or substitution at positions 152, 256, and 520. In some embodiments, the engineered nuclease comprises a replacement or substitution at positions 152, 256, and 526. In some embodiments, the engineered nuclease comprises a replacement or substitution at positions 152, 256, and 775. In some embodiments, the engineered nuclease comprises a replacement or substitution at positions 152, 515, and 520. In some embodiments, the engineered nuclease comprises a replacement or substitution at positions 152, 515, and 526. In some embodiments, the engineered nuclease comprises a replacement or substitution at positions 152, 515, and 775. In some embodiments, the engineered nuclease comprises a replacement or substitution at positions 152, 520, and 526. In some embodiments, the engineered nuclease comprises a replacement or substitution at positions 152, 520, and 775. In some embodiments, the engineered nuclease comprises a replacement or substitution at positions 152, 526, and 775. In some embodiments, the engineered nuclease comprises a replacement or substitution at positions 256, 515, and 520. In some embodiments, the engineered nuclease comprises a replacement or substitution at positions 256, 515, and 526. In some embodiments, the engineered nuclease comprises a replacement or substitution at positions 256, 515, and 775. In some embodiments, the engineered nuclease comprises a replacement or substitution at positions 256, 520, and 526. In some embodiments, the engineered nuclease comprises a replacement or substitution at positions 256, 520, and 775. In some embodiments, the engineered nuclease comprises a replacement or substitution at positions 256, 526, and 775. In some embodiments, the engineered nuclease comprises a replacement or substitution at positions 515, 520, and 526. In some embodiments, the engineered nuclease comprises a replacement or substitution at positions 515, 520, and 775. In some embodiments, the engineered nuclease comprises a replacement or substitution at positions 515, 526, and 775. In some embodiments, the engineered nuclease comprises a replacement or substitution at positions 520, 526 and 775.

In some embodiments, the amino acid sequence lacks one or more of the following features: E at position 152, N at position 256, N at position 515, D at position 520, K at position 526, and Q at position 775. In some embodiments, the replacement at position 152 is an E152R replacement. In some embodiments, the replacement at position 256 is a N256A replacement. In some embodiments, the replacement at position 515 is an N515R replacement. In some embodiments, the replacement at 520 is a D520R replacement. In some embodiments, the replacement at 526 is a K526R replacement. In some embodiments, the replacement at 775 is a Q775L replacement.

In some embodiments, the one or more amino acid replacements is selected from the group consisting of E152R, N256A, N515R, D520R, K526R and Q775L, in reference to SEQ ID NO: 18. In some embodiments, the one or more amino acid replacements comprises an E152R replacement, and N256A replacement, an N515R replacement, an D520R replacement, an K526R replacement, or an Q775L replacement, in reference to SEQ ID NO: 18.

In some embodiments, an engineered nuclease comprises an amino acid sequence with at least 90% (e.g., at least 90%, at least 91%, at least 92%, at least 93%, at least 94%, at least 95%, at least 96%, at least 97%, at least 98%, at least 99%) to SEQ ID NO: 19 with one or more amino acid replacements corresponding to positions selected from the group consisting of 154, 259, 531, 536, 542, and 802. In some embodiments, the engineered nuclease comprises a replacement or substitution at position 154. In some embodiments, the engineered nuclease comprises a replacement or substitution at position 259. In some embodiments, the engineered nuclease comprises a replacement or substitution at position 531. In some embodiments, the engineered nuclease comprises a replacement or substitution at position 536. In some embodiments, the engineered nuclease comprises a replacement or substitution at position 542. In some embodiments, the engineered nuclease comprises a replacement or substitution at position 802.

In some embodiments, the engineered nuclease comprises two amino acid replacements at positions selected from the group consisting of 154, 259, 531, 536, 542, and 802, in reference to SEQ ID NO: 19. In some embodiments, the engineered nuclease comprises a replacement or substitution at positions 154 and 259. In some embodiments, the engineered nuclease comprises a replacement or substitution at positions 154 and 531. In some embodiments, the engineered nuclease comprises a replacement or substitution at positions 154 and 536. In some embodiments, the engineered nuclease comprises a replacement or substitution at positions 154 and 542. In some embodiments, the engineered nuclease comprises a replacement or substitution at positions 154 and 802. In some embodiments, the engineered nuclease comprises a replacement or substitution at positions 259 and 531. In some embodiments, the engineered nuclease comprises a replacement or substitution at positions 259 and 536. In some embodiments, the engineered nuclease comprises a replacement or substitution at positions 259 and 542. In some embodiments, the engineered nuclease comprises a replacement or substitution at positions 259 and 802. In some embodiments, the engineered nuclease comprises a replacement or substitution at positions 531 and 536. In some embodiments, the engineered nuclease comprises a replacement or substitution at positions 531 and 542. In some embodiments, the engineered nuclease comprises a replacement or substitution at positions 531 and 802. In some embodiments, the engineered nuclease comprises a replacement or substitution at positions 536 and 542. In some embodiments, the engineered nuclease comprises a replacement or substitution at positions 536 and 802. In some embodiments, the engineered nuclease comprises a replacement or substitution at positions 542 and 802.

In some embodiments, the engineered nuclease comprises three amino acid replacements at positions selected from the group consisting of 154, 259, 531, 536, 542, and 802, in reference to SEQ ID NO: 19. In some embodiments, the engineered nuclease comprises a replacement or substitution at positions 154, 259, and 531. In some embodiments, the engineered nuclease comprises a replacement or substitution at positions 154, 259, and 536. In some embodiments, the engineered nuclease comprises a replacement or substitution at positions 154, 259, and 542. In some embodiments, the engineered nuclease comprises a replacement or substitution at positions 154, 259, and 802. In some embodiments, the engineered nuclease comprises a replacement or substitution at positions 154, 531, and 536. In some embodiments, the engineered nuclease comprises a replacement or substitution at positions 154, 531, and 542. In some embodiments, the engineered nuclease comprises a replacement or substitution at positions 154, 531, and 802. In some embodiments, the engineered nuclease comprises a replacement or substitution at positions 154, 536, and 542. In some embodiments, the engineered nuclease comprises a replacement or substitution at positions 154, 536, and 802. In some embodiments, the engineered nuclease comprises a replacement or substitution at positions 154, 542, and 802. In some embodiments, the engineered nuclease comprises a replacement or substitution at positions 259, 531, and 536. In some embodiments, the engineered nuclease comprises a replacement or substitution at positions 259, 531, and 542. In some embodiments, the engineered nuclease comprises a replacement or substitution at positions 259, 531, and 802. In some embodiments, the engineered nuclease comprises a replacement or substitution at positions 259, 536, and 542. In some embodiments, the engineered nuclease comprises a replacement or substitution at positions 259, 536, and 802. In some embodiments, the engineered nuclease comprises a replacement or substitution at positions 259, 542, and 802. In some embodiments, the engineered nuclease comprises a replacement or substitution at positions 531, 536, and 542. In some embodiments, the engineered nuclease comprises a replacement or substitution at positions 531, 536, and 802. In some embodiments, the engineered nuclease comprises a replacement or substitution at positions 531, 542, and 802. In some embodiments, the engineered nuclease comprises a replacement or substitution at positions 536, 542 and 802.

In some embodiments, the amino acid sequence lacks one or more of the following features: T at position 154, N at position 259, N at position 531, G at position 536, K at position 542, and S at position 802. In some embodiments, the replacement at position 154 is an T154R replacement. In some embodiments, the replacement at position 259 is a N259A replacement. In some embodiments, the replacement at position 531 is an N531R replacement. In some embodiments, the replacement at 536 is a G536R replacement. In some embodiments, the replacement at 542 is a K542R replacement. In some embodiments, the replacement at 802 is a S802L replacement.

In some embodiments, the one or more amino acid replacements is selected from the group consisting of T154R, N259A, N531R, G536R, K542R and S802L, in reference to SEQ ID NO: 19. In some embodiments, the one or more amino acid replacements comprises an T154R replacement, and N259A replacement, an N531R replacement, an G536R replacement, an K542R replacement, or an S802L replacement, in reference to SEQ ID NO: 19.

In some embodiments, an engineered nuclease comprises an amino acid sequence with at least 90% (e.g., at least 90%, at least 91%, at least 92%, at least 93%, at least 94%, at least 95%, at least 96%, at least 97%, at least 98%, at least 99%) to SEQ ID NO: 20 with one or more amino acid replacements corresponding to positions selected from the group consisting of 155, 261, 540, 545, and 551. In some embodiments, the engineered nuclease comprises a replacement or substitution at position 155. In some embodiments, the engineered nuclease comprises a replacement or substitution at position 261. In some embodiments, the engineered nuclease comprises a replacement or substitution at position 540. In some embodiments, the engineered nuclease comprises a replacement or substitution at position 545. In some embodiments, the engineered nuclease comprises a replacement or substitution at position 551.

In some embodiments, the engineered nuclease comprises two amino acid replacements at positions selected from the group consisting of 155, 261, 540, 545, and 551, in reference to SEQ ID NO: 20. In some embodiments, the engineered nuclease comprises a replacement or substitution at positions 155 and 261. In some embodiments, the engineered nuclease comprises a replacement or substitution at positions 155 and 540. In some embodiments, the engineered nuclease comprises a replacement or substitution at positions 155 and 545. In some embodiments, the engineered nuclease comprises a replacement or substitution at positions 155 and 551. In some embodiments, the engineered nuclease comprises a replacement or substitution at positions 261 and 540. In some embodiments, the engineered nuclease comprises a replacement or substitution at positions 261 and 545. In some embodiments, the engineered nuclease comprises a replacement or substitution at positions 261 and 551. In some embodiments, the engineered nuclease comprises a replacement or substitution at positions 540 and 545. In some embodiments, the engineered nuclease comprises a replacement or substitution at positions 540 and 551. In some embodiments, the engineered nuclease comprises a replacement or substitution at positions 545 and 551.

In some embodiments, the engineered nuclease comprises three amino acid replacements at positions selected from the group consisting of 155, 261, 540, 545, and 551, in reference to SEQ ID NO: 20. In some embodiments, the engineered nuclease comprises a replacement or substitution at positions 155, 261, and 540. In some embodiments, the engineered nuclease comprises a replacement or substitution at positions 155, 261, and 545. In some embodiments, the engineered nuclease comprises a replacement or substitution at positions 155, 261, and 551. In some embodiments, the engineered nuclease comprises a replacement or substitution at positions 155, 540, and 545. In some embodiments, the engineered nuclease comprises a replacement or substitution at positions 155, 540, and 551. In some embodiments, the engineered nuclease comprises a replacement or substitution at positions 155, 545, and 551. In some embodiments, the engineered nuclease comprises a replacement or substitution at positions 261, 540, and 545. In some embodiments, the engineered nuclease comprises a replacement or substitution at positions 261, 540, and 551. In some embodiments, the engineered nuclease comprises a replacement or substitution at positions 261, 545, and 551. In some embodiments, the engineered nuclease comprises a replacement or substitution at positions 540, 545, and 551.

In some embodiments, the amino acid sequence lacks one or more of the following features: E at position 155, N at position 261, N at position 540, G at position 545, and K at position 551. In some embodiments, the replacement at position 155 is an E155R replacement. In some embodiments, the replacement at position 261 is a N261A replacement. In some embodiments, the replacement at position 540 is an N540R replacement. In some embodiments, the replacement at 545 is a G545R replacement. In some embodiments, the replacement at 551 is a K551R replacement. In some embodiments, the one or more amino acid replacements is selected from the group consisting of E155R, N261A, N540R, G545R, and K551R, in reference to SEQ ID NO: 20. In some embodiments, the one or more amino acid replacements comprises an E155R replacement, an N261A replacement, an N540R replacement, a G545R replacement, or a K551R replacement, in reference to SEQ ID NO: 20.

In some embodiments, an engineered nuclease comprises an amino acid sequence with at least 90% (e.g., at least 90%, at least 91%, at least 92%, at least 93%, at least 94%, at least 95%, at least 96%, at least 97%, at least 98%, at least 99%) to SEQ ID NO: 21 with one or more amino acid replacements corresponding to positions selected from the group consisting of 155, 264, 542, 547, and 553. In some embodiments, the engineered nuclease comprises a replacement or substitution at position 155. In some embodiments, the engineered nuclease comprises a replacement or substitution at position 264. In some embodiments, the engineered nuclease comprises a replacement or substitution at position 542. In some embodiments, the engineered nuclease comprises a replacement or substitution at position 547. In some embodiments, the engineered nuclease comprises a replacement or substitution at position 553.

In some embodiments, the engineered nuclease comprises two amino acid replacements at positions selected from the group consisting of 155, 264, 542, 547, and 553, in reference to SEQ ID NO: 21. In some embodiments, the engineered nuclease comprises a replacement or substitution at positions 155 and 264. In some embodiments, the engineered nuclease comprises a replacement or substitution at positions 155 and 542. In some embodiments, the engineered nuclease comprises a replacement or substitution at positions 155 and 547. In some embodiments, the engineered nuclease comprises a replacement or substitution at positions 155 and 553. In some embodiments, the engineered nuclease comprises a replacement or substitution at positions 264 and 542. In some embodiments, the engineered nuclease comprises a replacement or substitution at positions 264 and 547. In some embodiments, the engineered nuclease comprises a replacement or substitution at positions 264 and 553. In some embodiments, the engineered nuclease comprises a replacement or substitution at positions 542 and 547. In some embodiments, the engineered nuclease comprises a replacement or substitution at positions 542 and 553. In some embodiments, the engineered nuclease comprises a replacement or substitution at positions 547 and 553.

In some embodiments, the engineered nuclease comprises three amino acid replacements at positions selected from the group consisting of 155, 264, 542, 547, and 553, in reference to SEQ ID NO: 21. In some embodiments, the engineered nuclease comprises a replacement or substitution at positions 155, 264, and 542. In some embodiments, the engineered nuclease comprises a replacement or substitution at positions 155, 264, and 547. In some embodiments, the engineered nuclease comprises a replacement or substitution at positions 155, 264, and 553. In some embodiments, the engineered nuclease comprises a replacement or substitution at positions 155, 542, and 547. In some embodiments, the engineered nuclease comprises a replacement or substitution at positions 155, 542, and 553. In some embodiments, the engineered nuclease comprises a replacement or substitution at positions 155, 547, and 553. In some embodiments, the engineered nuclease comprises a replacement or substitution at positions 264, 542, and 547. In some embodiments, the engineered nuclease comprises a replacement or substitution at positions 264, 542, and 553. In some embodiments, the engineered nuclease comprises a replacement or substitution at positions 264, 547, and 553. In some embodiments, the engineered nuclease comprises a replacement or substitution at positions 542, 547, and 553.

In some embodiments, the amino acid sequence lacks one or more of the following features: K at position 155, N at position 264, N at position 542, D at position 547, and K at position 553. In some embodiments, the replacement at position 155 is an K155R replacement. In some embodiments, the replacement at position 264 is a N264A replacement. In some embodiments, the replacement at position 542 is an N542R replacement. In some embodiments, the replacement at 547 is a D547R replacement. In some embodiments, the replacement at 553 is a K553R replacement. In some embodiments, the one or more amino acid replacements is selected from the group consisting of K155R, N264A, N542R, D547R and K553R, in reference to SEQ ID NO: 21. In some embodiments, the one or more amino acid replacements comprises an K155R replacement, an N264A replacement, an N542R replacement, a D547R replacement, or a K553R replacement, in reference to SEQ ID NO: 21.

In some embodiments, an engineered nuclease comprises an amino acid sequence with at least 90% (e.g., at least 90%, at least 91%, at least 92%, at least 93%, at least 94%, at least 95%, at least 96%, at least 97%, at least 98%, at least 99%) to SEQ ID NO: 22 with one or more amino acid replacements corresponding to positions selected from the group consisting of 175, 280, 543, 548, and 554. In some embodiments, the engineered nuclease comprises a replacement or substitution at position 175. In some embodiments, the engineered nuclease comprises a replacement or substitution at position 280. In some embodiments, the engineered nuclease comprises a replacement or substitution at position 543. In some embodiments, the engineered nuclease comprises a replacement or substitution at position 548. In some embodiments, the engineered nuclease comprises a replacement or substitution at position 554.

In some embodiments, the engineered nuclease comprises two amino acid replacements at positions selected from the group consisting of 175, 280, 543, 548, and 554, in reference to SEQ ID NO: 22. In some embodiments, the engineered nuclease comprises a replacement or substitution at positions 175 and 280. In some embodiments, the engineered nuclease comprises a replacement or substitution at positions 175 and 543. In some embodiments, the engineered nuclease comprises a replacement or substitution at positions 175 and 548. In some embodiments, the engineered nuclease comprises a replacement or substitution at positions 175 and 554. In some embodiments, the engineered nuclease comprises a replacement or substitution at positions 280 and 543. In some embodiments, the engineered nuclease comprises a replacement or substitution at positions 280 and 548. In some embodiments, the engineered nuclease comprises a replacement or substitution at positions 280 and 554. In some embodiments, the engineered nuclease comprises a replacement or substitution at positions 543 and 548. In some embodiments, the engineered nuclease comprises a replacement or substitution at positions 543 and 554. In some embodiments, the engineered nuclease comprises a replacement or substitution at positions 548 and 554.

In some embodiments, the engineered nuclease comprises three amino acid replacements at positions selected from the group consisting of 175, 280, 543, 548, and 554, in reference to SEQ ID NO: 22. In some embodiments, the engineered nuclease comprises a replacement or substitution at positions 175, 280, and 543. In some embodiments, the engineered nuclease comprises a replacement or substitution at positions 175, 280, and 548. In some embodiments, the engineered nuclease comprises a replacement or substitution at positions 175, 280, and 554. In some embodiments, the engineered nuclease comprises a replacement or substitution at positions 175, 543, and 548. In some embodiments, the engineered nuclease comprises a replacement or substitution at positions 175, 543, and 554. In some embodiments, the engineered nuclease comprises a replacement or substitution at positions 175, 548, and 554. In some embodiments, the engineered nuclease comprises a replacement or substitution at positions 280, 543, and 548. In some embodiments, the engineered nuclease comprises a replacement or substitution at positions 280, 543, and 554. In some embodiments, the engineered nuclease comprises a replacement or substitution at positions 280, 548, and 554. In some embodiments, the engineered nuclease comprises a replacement or substitution at positions 543, 548, and 554.

In some embodiments, the amino acid sequence lacks one or more of the following features: K at position 175, N at position 280, C at position 543, N at position 548, and K at position 554. In some embodiments, the replacement at position 175 is an K175R replacement. In some embodiments, the replacement at position 280 is a N280A replacement. In some embodiments, the replacement at position 543 is an C543R replacement. In some embodiments, the replacement at 548 is a N548R replacement. In some embodiments, the replacement at 554 is a K554R replacement. In some embodiments, the one or more amino acid replacements is selected from the group consisting of K175R, N280A, C543R, N548R and K554R, in reference to SEQ ID NO: 22. In some embodiments, the one or more amino acid replacements comprises an K175R replacement, an N280A replacement, an C543R replacement, a N548R replacement, or a K554R replacement, in reference to SEQ ID NO: 22.

In some embodiments, an engineered nuclease comprises an amino acid sequence with at least 90% (e.g., at least 90%, at least 91%, at least 92%, at least 93%, at least 94%, at least 95%, at least 96%, at least 97%, at least 98%, at least 99%) to SEQ ID NO: 23 with one or more amino acid replacements corresponding to positions selected from the group consisting of 171, 277, 558, 563, 569, and 827. In some embodiments, the engineered nuclease comprises a replacement or substitution at position 171. In some embodiments, the engineered nuclease comprises a replacement or substitution at position 277. In some embodiments, the engineered nuclease comprises a replacement or substitution at position 558. In some embodiments, the engineered nuclease comprises a replacement or substitution at position 563. In some embodiments, the engineered nuclease comprises a replacement or substitution at position 569. In some embodiments, the engineered nuclease comprises a replacement or substitution at position 827.

In some embodiments, the engineered nuclease comprises two amino acid replacements at positions selected from the group consisting of 171, 277, 558, 563, 569, and 827, in reference to SEQ ID NO: 23. In some embodiments, the engineered nuclease comprises a replacement or substitution at positions 171 and 277. In some embodiments, the engineered nuclease comprises a replacement or substitution at positions 171 and 558. In some embodiments, the engineered nuclease comprises a replacement or substitution at positions 171 and 563. In some embodiments, the engineered nuclease comprises a replacement or substitution at positions 171 and 569. In some embodiments, the engineered nuclease comprises a replacement or substitution at positions 171 and 827. In some embodiments, the engineered nuclease comprises a replacement or substitution at positions 277 and 558. In some embodiments, the engineered nuclease comprises a replacement or substitution at positions 277 and 563. In some embodiments, the engineered nuclease comprises a replacement or substitution at positions 277 and 569. In some embodiments, the engineered nuclease comprises a replacement or substitution at positions 277 and 827. In some embodiments, the engineered nuclease comprises a replacement or substitution at positions 558 and 563. In some embodiments, the engineered nuclease comprises a replacement or substitution at positions 558 and 569. In some embodiments, the engineered nuclease comprises a replacement or substitution at positions 558 and 827. In some embodiments, the engineered nuclease comprises a replacement or substitution at positions 563 and 569. In some embodiments, the engineered nuclease comprises a replacement or substitution at positions 563 and 827. In some embodiments, the engineered nuclease comprises a replacement or substitution at positions 569 and 827.

| | In some embodiments, the engineered nuclease comprises three amino acid replacements at positions selected from the group consisting of 171, 277, 558, 563, 569, and 827, in reference to SEQ ID NO: 23. In some embodiments, the engineered nuclease comprises a replacement or substitution at positions 171, 277, and 558. In some embodiments, the engineered nuclease comprises a replacement or substitution at positions 171, 277, and 563. In some embodiments, the engineered nuclease comprises a replacement or substitution at positions 171, 277, and 569. In some embodiments, the engineered nuclease comprises a replacement or substitution at positions 171, 277, and 827. In some embodiments, the engineered nuclease comprises a replacement or substitution at positions 171, 558, and 563. In some embodiments, the engineered nuclease comprises a replacement or substitution at positions 171, 558, and 569. In some embodiments, the engineered nuclease comprises a replacement or substitution at positions 171, 558, and 827. In some embodiments, the engineered nuclease comprises a replacement or substitution at positions 171, 563, and 569. In some embodiments, the engineered nuclease comprises a replacement or substitution at positions 171, 563, and 827. In some embodiments, the engineered nuclease comprises a replacement or substitution at positions 171, 569, and 827. In some embodiments, the engineered nuclease comprises a replacement or substitution at positions 277, 558, and 563. In some embodiments, the engineered nuclease comprises a replacement or substitution at positions 277, 558, and 569. In some embodiments, the engineered nuclease comprises a replacement or substitution at positions 277, 558, and 827. In some embodiments, the engineered nuclease comprises a replacement or substitution at positions 277, 563, and 569. In some embodiments, the engineered nuclease comprises a replacement or substitution at positions 277, 563, and 827. In some embodiments, the engineered nuclease comprises a replacement or substitution at positions 277, 569, and 827. In some embodiments, the engineered nuclease comprises a replacement or substitution at positions 558, 563, and 569. In some embodiments, the engineered nuclease comprises a replacement or substitution at positions 558, 563, and 827. In some embodiments, the engineered nuclease comprises a replacement or substitution at positions 558, 569, and 827. In some embodiments, the engineered nuclease comprises a replacement or substitution at positions 563, 569 and 827.

In some embodiments, the amino acid sequence lacks one or more of the following features: E at position 171, N at position 277, N at position 558, N at position 563, K at position 569 and Q at position 827. In some embodiments, the replacement at position 171 is an E171R replacement. In some embodiments, the replacement at position 277 is a N277A replacement. In some embodiments, the replacement at position 558 is an N558R replacement. In some embodiments, the replacement at 563 is a D563R replacement. In some embodiments, the replacement at 569 is a K569R replacement. In some embodiments, the replacement at 827 is a Q827L replacement.

In some embodiments, the one or more amino acid replacements is selected from the group consisting of E171R, N277A, N558R, D563R, K569R and Q827L, in reference to SEQ ID NO: 23. In some embodiments, the one or more amino acid replacements comprises an E171R replacement, and N277A replacement, an N558R replacement, an D563R replacement, an K569R replacement, or an Q827L replacement, in reference to SEQ ID NO: 23.

In some embodiments, the engineered nuclease comprises one or more (e.g., one, two, three) of SEQ ID NOs: 419-487. In some embodiments, the engineered nuclease comprises one of SEQ ID NOs: 419-487. In some embodiments, the engineered nuclease comprises one or more (e.g., one, two, three) of SEQ ID NOs: 419-421. In some embodiments, the engineered nuclease comprises one or more of SEQ ID NOs: 422-424. In some embodiments, the engineered nuclease comprises one or more of SEQ ID NOs. 425-427. In some embodiments, the engineered nuclease comprises one or more of SEQ ID NOs: 428-430. In some embodiments, the engineered nuclease comprises one or more of SEQ ID NOs. 431-433. In some embodiments, the engineered nuclease comprises one or more of SEQ ID NOs: 434-436. In some embodiments, the engineered nuclease comprises one or more of SEQ ID NOs. 437-439. In some embodiments, the engineered nuclease comprises one or more of SEQ ID NOs: 440-442. In some embodiments, the engineered nuclease comprises one or more of SEQ ID NOs: 443-445. In some embodiments, the engineered nuclease comprises one or more of SEQ ID NOs: 446-448. In some embodiments, the engineered nuclease comprises one or more of SEQ ID NOs: 449-451. In some embodiments, the engineered nuclease comprises one or more of SEQ ID NOs: 452-454. In some embodiments, the engineered nuclease comprises one or more of SEQ ID NOs: 455-457. In some embodiments, the engineered nuclease comprises one or more of SEQ ID NOs: 458-460. In some embodiments, the engineered nuclease comprises one or more of SEQ ID NOs: 461-463. In some embodiments, the engineered nuclease comprises one or more of SEQ ID NOs: 464-466. In some embodiments, the engineered nuclease comprises one or more of SEQ ID NOs: 467-469. In some embodiments, the engineered nuclease comprises one or more of SEQ ID NOs: 470-472. In some embodiments, the engineered nuclease comprises one or more of SEQ ID NOs: 473-475. In some embodiments, the engineered nuclease comprises one or more of SEQ ID NOs: 476-478. In some embodiments, the engineered nuclease comprises one or more of SEQ ID NOs: 479-481. In some embodiments, the engineered nuclease comprises one or more of SEQ ID NOs: 482-484. In some embodiments, the engineered nuclease comprises one or more of SEQ ID NOs: 485-487.

In some embodiments, the engineered nuclease does not contain an amino acid sequence having SEQ ID NO: 488

The engineered nuclease may further comprise a nuclear localization sequence (NLS). The nuclear localization sequence may be appended, for example, to the N-terminus, the C-terminus, or a combination thereof. The nuclease localization sequence may be inserted within the coding sequence. In some embodiments, the engineered nuclease comprises two or more NLSs. The two or more NLSs may be in tandem, separated by a linker, at either end terminus of the protein, or one or more may be embedded in the protein.

The nuclear localization sequence may comprise any amino acid sequence known in the art to functionally tag or direct a protein for import into a cell's nucleus (e.g., for nuclear transport). Usually, a nuclear localization sequence comprises one or more positively charged amino acids, such as lysine and arginine.

In some embodiments, the NLS is a monopartite sequence. A monopartite NLS comprise a single cluster of positively charged or basic amino acids. In some embodiments, the monopartite NLS comprises a sequence of K-K/R-X-K/R, wherein X can be any amino acid. Exemplary monopartite NLS sequences include those from the SV40 large T-antigen, c-Myc, and TUS-proteins. In select embodiments, the NLS sequence comprises the c-Myc NLS; PAAKRVKLD (SEQ ID NO: 30).

In some embodiments, the NLS is a bipartite sequence. Bipartite NLSs comprise two clusters of basic amino acids, separated by a spacer of about 9-12 amino acids. Exemplary bipartite NLSs include the nuclear localization sequences of nucleoplasmin, EGL-12, or bipartite SV40. In select embodiments, the NLS comprises the NLS of nucleoplasmin, KR[PAATKKAGQA]KKKK (SEQ ID NO: 31).

The engineered nuclease may further comprise an epitope tag (e.g., 3×FLAG tag, an HA tag, a Myc tag, and the like). In some embodiments, the epitope tag may be adjacent, either upstream or downstream, to a nuclear localization sequence. The epitope tags may be at the N-terminus, a C-terminus, or a combination thereof of the corresponding protein.

The engineered nuclease may be part of a fusion protein comprising another protein or protein domain. For example, the engineered nuclease may be fused to another protein or protein domain that provides for tagging or visualization (e.g., GFP). The engineered nuclease may be fused to a protein or protein domain that has another functionality or activity useful to target to certain DNA sequences (e.g., nuclease activity such as that provide by FokI nuclease, protein modification activity such as histone modification activity including acetylation or deacetylation or demethylation or methyltransferase activity, transcription modulation activity such as activity of a transcriptional activator or repressor, base editing activity such as deaminase activity, DNA modifying activity such as DNA methylation activity, and the like).

In some embodiments, the engineered nuclease may be fused with one or more (e.g., two, three, four, or more) protein transduction domains or PTDs, also known as a CPP-cell penetrating peptide. A protein transduction domains is a polypeptide, polynucleotide, carbohydrate, or organic or inorganic compound that facilitates traversing a lipid bilayer, micelle, cell membrane, organelle membrane, or vesicle membrane. A PTD attached to another molecule, facilitates the molecule traversing a membrane, for example going from extracellular space to intracellular space, or cytosol to within an organelle. In some embodiments, a PTD is covalently linked to a terminus of the nuclease (e.g., N-terminus, C-terminus, or both). In some embodiments, the PTD is inserted internally at a suitable insertion site. Examples of PTDs include but are not limited to a minimal undecapeptide protein transduction domain (corresponding to residues 47-57 of HIV-1 TAT comprising); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7): 1732-1737); a truncated human calcitonin peptide (Trehin et al. (2004) Pharm. Research 21:1248-1256); polylysine (Wender et al. (2000) Proc. Natl. Acad. Sci. USA 97:13003-13008); Transportan, and the like.

The engineered nuclease may be fused via a linker polypeptide. The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers can be produced by using synthetic, linker-encoding oligonucleotides to couple the proteins, or can be encoded by a nucleic acid sequence encoding the fusion protein. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use, including but not limited to, glycine-serine polymers, glycine-alanine polymers, and alanine-serine polymers.

### Compositions and Systems

Disclosed herein are compositions comprising an engineered nuclease, as described herein, or a nucleic acid molecule comprising a sequence encoding the engineered nuclease.

Also disclosed herein are compositions comprising a nuclease comprising an amino acid sequence having at least 70% identity to any of SEQ ID NOs: 1-23 or 32-35 or a nucleic acid molecule comprising a sequence encoding the nuclease. Also disclosed herein are compositions comprising a nuclease comprising an amino acid sequence having about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or 100% identity to any of SEQ ID NOs: 1-23 or 32-35 or a nucleic acid molecule comprising a sequence encoding the nuclease. In some embodiments, the nuclease comprises an amino acid sequence having at least 90% (e.g., 90%, 91%, 92%, 93%, 94%, 95%, 96%, 97%, 98%, 99%, or 100%) identity to any of SEQ ID NOs. 1-23 or 32-35. The amino acid sequence of the nuclease may have less than 50% sequence identity with SEQ ID NO: 24. In some embodiments, the nuclease comprises an amino acid sequence selected from the group consisting of SEQ ID NOs: 1-23, 26-29, and 32-236.

Further disclosed herein are systems for modifying a target nucleic acid comprising an engineered nuclease as described herein or a nucleic acid molecule comprising a sequence encoding the engineered nuclease. Also disclosed herein are systems for modifying a target nucleic acid comprising a nuclease comprising an amino acid sequence having at least 70% identity to any of SEQ ID NOs: 1-23 or 32-35 or a nucleic acid molecule comprising a sequence encoding the nuclease. Also disclosed herein are systems for modifying a target nucleic acid comprising an amino acid sequence having about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or 100% identity to any of SEQ ID NOs: 1-23 or 32-35 or a nucleic acid molecule comprising a sequence encoding the nuclease. In some embodiments, the nuclease comprises an amino acid sequence having at least 90% identity to any of SEQ ID NOs: 1-23 or 32-35. The amino acid sequence of the nuclease may have less than 50% sequence identity with SEQ ID NO: 24. In some embodiments, the nuclease comprises an amino acid sequence selected from the group consisting of SEQ ID NOs: 1-23, 26-29, and 32-236.

In some embodiments, the components of the system may be in the form of a composition. In some embodiments, the components of the present compositions or systems may be mixed, individually or in any combination, with a carrier which are also within the scope of the present disclosure. Exemplary carriers include buffers, antioxidants, preservatives, carbohydrates, surfactants, and the like.

In some embodiments, the nuclease in the compositions or systems may further comprise one or more nuclear localization sequences (NLS), as described elsewhere herein. The nuclear localization sequence(s) may be, for example, appended to the N-terminus, appended the C-terminus, inserted within the coding sequence, or a combination thereof. KR[PAATKKAGQA]KKKK (SEQ ID NO: 31). In some embodiments, the nuclease may further comprise an epitope tag, as described elsewhere herein. In some embodiments, the epitope tag may be adjacent, either upstream or downstream, to a nuclear localization sequence. The epitope tags may be at the N-terminus, a C-terminus, or a combination thereof of the corresponding protein. In some embodiments, the nuclease may be part of a fusion protein comprising another protein or protein domain, as described elsewhere herein.

The compositions or systems disclosed herein may further comprise at least one gRNA, or a nucleic acid encoding the at least one gRNA. In instances when the composition or system comprises more than one gRNA, each may be encoded the same or different nucleic acid as the other gRNA. In some embodiments, the at least one gRNA is complementary to at least a portion of the target nucleic acid sequence.

The gRNA may be a crRNA, crRNA/tracrRNA (or single guide RNA, sgRNA). The terms “gRNA,” “guide RNA” and “CRISPR guide sequence” may be used interchangeably throughout and refer to a nucleic acid comprising a sequence that determines the sequence specificity of the nuclease. A gRNA hybridizes to (complementary to, partially or completely) a target nucleic acid sequence (e.g., the genome in a host cell). In some embodiments, the at least one gRNA is encoded in a CRISPR RNA (crRNA) array.

The gRNA or portion thereof that hybridizes to the target nucleic acid (a target site) may be between 15-40 nucleotides in length. In some embodiments, the gRNA sequence that hybridizes to the target nucleic acid is 15, 16, 17, 18, 19, 20, 21, 22, 23, 24, 25, 26, 27, 28, 29, 30, 31, 32, 33, 34, 35, 36, 37, 38, 39, or 40 nucleotides in length. gRNAs or sgRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer).

To facilitate gRNA design, many computational tools have been developed (See Prykhozhij et al. (PLOS ONE, 10(3): (2015)); Zhu et al. (PLOS ONE, 9(9) (2014)); Xiao et al. (Bioinformatics. January 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

In addition to a sequence that binds to a target nucleic acid, in some embodiments, the gRNA may also comprise a scaffold sequence (e.g., tracrRNA). In some embodiments, such a chimeric gRNA may be referred to as a single guide RNA (sgRNA). Exemplary scaffold sequences will be evident to one of skill in the art and can be found, for example, in Jinek, et al. Science (2012) 337(6096):816-821, and Ran, et al. Nature Protocols (2013) 8:2281-2308, incorporated herein by reference in their entireties.

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence.

In some embodiments, the gRNA sequence is at least 50%, 55%, 60%, 65%, 70%, 75%, 80%, 85%, 90%, 95%, 96%, 97%, 98%, 99%, or at least 100% complementary to a target nucleic acid. In some embodiments, the gRNA sequence is at least 50%, 55%, 60%, 65%, 70%, 75%, 80%, 85%, 90%, 95%, 96%, 97%, 98%, 99%, or at least 100% complementary to the 3′ end of the target nucleic acid (e.g., the last 5, 6, 7, 8, 9, or 10 nucleotides of the 3′ end of the target nucleic acid).

The gRNA may be a non-naturally occurring gRNA.

The target sequence may or may not be flanked by a protospacer adjacent motif (PAM) sequence. In certain embodiments, a nucleic acid-guided nuclease can only cleave a target sequence if an appropriate PAM is present, see, for example Doudna et al., Science, 2014, 346(6213): 1258096, incorporated herein by reference. A PAM can be 5′ or 3′ of a target sequence. A PAM can be upstream or downstream of a target sequence. In one embodiment, the target sequence is immediately flanked on the 3′ end by a PAM sequence. A PAM can be 1, 2, 3, 4, 5, 6, 7, 8, 9, 10 or more nucleotides in length. In certain embodiments, a PAM is between 2-6 nucleotides in length. Sequence requirements for PAMs for any given nuclease can be determined using known methods, for example, the protocol of Walton et al. (Walton R T, et al., Science. 2020 Apr. 17,368(6488):290-296, incorporated herein by reference in its entirety) or as described in Example 8.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization. There may be mismatches distal from the PAM.

In some cases, the compositions or systems disclosed herein may further comprise a donor polynucleotide. For example, in applications in which it is desirable to insert a polynucleotide sequence into the genome where a target sequence is cleaved, a donor polynucleotide (a nucleic acid comprising a donor sequence) can also be provided to the cell. By a “donor sequence” or “donor polynucleotide” or “donor template” it is meant a nucleic acid sequence to be inserted at the site targeted by the nuclease (e.g., after dsDNA cleavage, after nicking a target DNA, after dual nicking a target DNA, and the like). In some cases, the donor sequence is provided to the cell as single-stranded DNA. In some cases, the donor template is provided to the cell as double-stranded DNA. It may be introduced into a cell in linear or circular form. If introduced in linear form, the ends of the donor sequence may be protected (e.g., from exonucleolytic degradation) by any convenient method and such methods are known to those of skill in the art. For example, one or more dideoxynucleotide residues can be added to the 3′ terminus of a linear molecule and/or self-complementary oligonucleotides can be ligated to one or both ends. A donor template can be introduced into a cell as part of a vector molecule having additional sequences such as, for example, replication origins, promoters and genes encoding antibiotic resistance. Moreover, donor template can be introduced as naked nucleic acid, as nucleic acid complexed with an agent such as a liposome or poloxamer, or can be delivered by viruses (e.g., adenovirus, AAV).

Also disclosed is a cell comprising the compositions or systems described herein. In some embodiments, the cell is a prokaryotic cell. In some embodiments, the cell is a eukaryotic cell. In some embodiments, the cell is a mammalian cell. In some embodiments, the cell is a human cell

### Nucleic Acids

Also disclosed herein are one or more nucleic acids encoding an engineered nuclease, as disclosed herein, a nuclease comprising an amino acid sequence having at least 70% identity (e.g., about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or 100% identity) to any of SEQ ID NOs: 1-23 or 32-35, the gRNA(s), and systems thereof. In some embodiments, the one or more nucleic acids comprise one or more messenger RNAs, one or more vectors, or any combination thereof. A single nucleic acid may encode the engineered nuclease or the nuclease and the at least one gRNA, or the engineered nuclease or the nuclease may be encoded on a separate nucleic acid from the at least one gRNA.

In some embodiments, the engineered nuclease or the nuclease is provided as a split-nuclease (e.g., a nuclease can in some cases be delivered as a split-nuclease, or a nucleic acid(s) encoding a split-nuclease) such that two separate proteins together form a functional nuclease. In some such cases the sequences that encode the two parts of the split-nuclease protein are present on the same vector. In some cases, they are present on separate vectors, e.g., as part of a vector system that encodes the nucleases, the gRNA(s), and systems thereof.

In certain embodiments, engineering the nucleases for use in eukaryotic cells may involve codon-optimization. It will be appreciated that changing native codons to those most frequently used in mammals allows for maximum expression of the system proteins in mammalian cells (e.g., human cells). Such modified nucleic acid sequences are commonly described in the art as “codon-optimized,” or as utilizing “mammalian-preferred” or “human-preferred” codons. In some embodiments, the nucleic acid sequence is considered codon-optimized if at least about 60% (e.g., 65%, 70%, 75%, 80%, 85%, 90%, 95%, or 98%) of the codons encoded therein are mammalian preferred codons.

The present disclosure also provides for DNA segments encoding an engineered nuclease, as disclosed herein, or a nuclease comprising an amino acid sequence having at least 70% identity (e.g., about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or 100% identity) to any of SEQ ID NOs: 1-23 or 32-35 and nucleic acids (e.g., gRNA) disclosed herein, vectors containing these segments and cells containing the vectors. The vectors may be used to propagate the segment in an appropriate cell and/or to allow expression from the segment (e.g., an expression vector). The person of ordinary skill in the art would be aware of the various vectors available for propagation and expression of a nucleic acid sequence.

The present disclosure further provides engineered, non-naturally occurring vectors and vector systems, which can encode one or more or all of the components of the present system. The vector(s) can be introduced into a cell that is capable of expressing the polypeptide encoded thereby, including any suitable prokaryotic or eukaryotic cell.

The vectors of the present disclosure may be delivered to a eukaryotic cell in a subject. Modification of the eukaryotic cells via the present system can take place in a cell culture, where the method comprises isolating the eukaryotic cell from a subject prior to the modification. In some embodiments, the method further comprises returning said eukaryotic cell and/or cells derived therefrom to the subject.

Viral and non-viral based gene transfer methods can be used to introduce nucleic acids encoding components of the present system into cells, tissues, or a subject. Such methods can be used to administer nucleic acids encoding components of the present system to cells in culture, or in a host organism. Non-viral vector delivery systems include DNA plasmids, cosmids, RNA (e.g., a transcript of a vector described herein), a nucleic acid, and a nucleic acid complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors.

In certain embodiments, plasmids that are non-replicative, or plasmids that can be cured by high temperature may be used, such that any or all of the necessary components of the composition or system may be removed from the cells under certain conditions. For example. this may allow for DNA integration by transforming bacteria of interest, but then being left with engineered strains that have no memory of the plasmids or vectors used for the integration.

A variety of viral constructs may be used to deliver the present composition or system (such as a nuclease and one or more gRNA(s)) to the targeted cells and/or a subject. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1): 33-40; and Walther W. and Stein U., 2000 Drugs, 60(2): 249-71, incorporated herein by reference.

In one embodiment, a DNA segment encoding an engineered nuclease, as disclosed herein, or a nuclease comprising an amino acid sequence having at least 70% identity (about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or 100% identity) to any of SEQ ID NOs: 1-23 or 32-35 is contained in a plasmid vector that allows expression of the protein and subsequent isolation and purification of the protein produced by the recombinant vector. Accordingly, the nucleases disclosed herein can be purified following expression, obtained by chemical synthesis, or obtained by recombinant methods.

To construct cells that express the present system, expression vectors for stable or transient expression of the system, or any of its components, may be constructed via methods as described herein or known in the art and introduced into cells. For example, nucleic acids encoding the components of the present system may be cloned into a suitable expression vector, such as a plasmid or a viral vector in operable linkage to a suitable promoter. The selection of expression vectors/plasmids/viral vectors should be suitable for integration and replication in eukaryotic cells.

In certain embodiments, vectors of the present disclosure can drive the expression of one or more sequences in prokaryotic cells. Promoters that may be used include T7 RNA polymerase promoters, constitutive E. coli promoters, and promoters that could be broadly recognized by transcriptional machinery in a wide range of bacterial organisms. The composition or system may be used with various bacterial hosts.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EFla (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), HI (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Different promoters and regulatory elements may be used to achieve proper balance (expression level ratio) between the components of the systems (e.g., the nuclease, the at least one gRNA). For example, in some cases a nucleic acid includes a promoters and regulatory elements that is operably linked to (and therefore regulates/modulates translation of) a sequence encoding the nuclease. In some cases, a subject nucleic acid includes a promoters and regulatory elements that is operably linked to a sequence encoding the gRNA. In some cases, the sequence encoding the nuclease and the sequence encoding the gRNA are both operably linked to the same promoters and regulatory elements.

A variety of promoter types are suitable for use. A promoter can be a constitutively active promoter (e.g., a promoter that is constitutively in an active/“ON” state), it may be an inducible promoter (e.g., a promoter whose state, active/“ON” or inactive/“OFF”, is controlled by an external stimulus, e.g., the presence of a particular temperature, compound, or protein), it may be a spatially restricted promoter (e.g., tissue specific promoter, cell type specific promoter, etc.), and it may be a temporally restricted promoter (e.g., the promoter is in the “ON” state or “OFF” state during specific stages of embryonic development or during specific stages of a biological process, e.g., hair follicle cycle in mice).

Inducible and tissue specific expression of RNA or proteins can be accomplished by placing the nucleic acid encoding such a molecule under the control of an inducible or tissue specific promoter/regulatory sequence. The vectors of the present disclosure may direct expression of the nucleic acid in a particular cell type (e.g., tissue-specific regulatory elements are used to express the nucleic acid) Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters and tumor-specific are available, for example from InvivoGen. In addition, promoters that are well known in the art can be induced in response to inducing agents such as metals, glucocorticoids, tetracycline, hormones, and the like, are also contemplated for use with the invention.

Examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter (see, e.g., EMBL HSENO2, X51956); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter; a serotonin receptor promoter (see, e.g., GenBank S62283); a tyrosine hydroxylase promoter (TH); a GnRH promoter; an L7 promoter; a DNMT promoter; an enkephalin; a myelin basic protein (MBP) promoter; a Ca2+-calmodulin-dependent protein kinase II-alpha (CamKIla) promoter; a CMV enhancer/platelet-derived growth factor-β promoter; and the like. Suitable liver-specific promoters can in some cases include, but are not limited to: TTR, Albumin, and AAT promoters. Suitable CNS-specific promoters can in some cases include, but are not limited to: Synapsin 1, BM88, CHNRB2, GFAP, and CAMK2a promoters. Suitable muscle-specific promoters can in some cases include, but are not limited to: MYOD1, MYLK2, SPc5-12 (synthetic), α-MHC, MLC-2, MCK, MHCK7, human cardiac troponin C (cInC) and desmin promoters. Adipocyte-specific spatially restricted promoters include, but are not limited to, aP2 gene promoter/enhancer, e.g., a region from −5.4 kb to +21 bp of a human aP2; a glucose transporter-4 (GLUT4), a fatty acid translocase (FAT/CD36) promoter; a stearoyl-CoA desaturase-1 (SCD1) promoter; a leptin promoter; an adiponectin promoter, an adipsin promoter; a resistin promoter; and the like. Cardiomyocyte-specific spatially restricted promoters include, but are not limited to, control sequences derived from the following genes: myosin light chain-2, α-myosin heavy chain, AE3, cardiac troponin C, cardiac actin, and the like. Smooth muscle-specific spatially restricted promoters include, but are not limited to, an SM22a promoter; a smoothelin promoter, an α-smooth muscle actin promoter; and the like. For example, a 0.4 kb region of the SM22a promoter, within which lie two CArG elements, has been shown to mediate vascular smooth muscle cell-specific. Photoreceptor-specific spatially restricted promoters include, but are not limited to, a rhodopsin promoter; a rhodopsin kinase promoter; a beta phosphodiesterase gene; a retinitis pigmentosa gene promoter; an interphotoreceptor retinoid-binding protein (IRBP) gene enhancer; an IRBP gene promoter; and the like.

Examples of inducible promoters include, but are not limited to, heat shock promoter, tetracycline-regulated promoter, steroid-regulated promoter, metal-regulated promoter, estrogen receptor-regulated promoter, etc. Inducible promoters can therefore be regulated by molecules including, but not limited to, doxycycline; an estrogen receptor; an estrogen receptor fusion; an estrogen analog; IPTG; etc. Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

Inducible promoters include sugar-inducible promoters (e.g., lactose-inducible promoters, arabinose-inducible promoters); amino acid-inducible promoters; alcohol-inducible promoters; and the like. Suitable promoters include, e.g., lactose-regulated systems (e.g., lactose operon systems, sugar-regulated systems, isopropyl-beta-D-thiogalactopyranoside (IPTG) inducible systems, arabinose regulated systems (e.g., arabinose operon systems, e.g., an ARA operon promoter, pBAD, PARA, portions thereof, combinations thereof and the like), synthetic amino acid regulated systems, fructose repressors, a tac promoter/operator (pTac), tryptophan promoters, PhoA promoters, recA promoters, proU promoters, cst-1 promoters, tetA promoters, cadA promoters, nar promoters, P_{L} promoters, espA promoters, and the like, or combinations thereof. In certain cases, a promoter comprises a Lac-Z, or portions thereof. In some cases, a promoter comprises a Lac operon, or portions thereof. In some cases, an inducible promoter comprises an ARA operon promoter, or portions thereof. In certain embodiments an inducible promoter comprises an arabinose promoter or portions thereof. An arabinose promoter can be obtained from any suitable bacteria. In some cases, an inducible promoter comprises an arabinose operon of E. coli or B. subtilis. In some cases, an inducible promoter is activated by the presence of a sugar or an analog thereof. Non-limiting examples of sugars and sugar analogs include lactose, arabinose (e.g., L-arabinose), glucose, sucrose, fructose, IPTG, and the like. Suitable promoters include a T7 promoter; a pBAD promoter; a lacIQ promoter; and the like. In some cases, the promoter is a J23119 promoter. Many bacterial promoters are known in the art; bacterial promoters can be found on the internet at parts(dot)igem(dot)org/promoters.

In some cases, the promoter is a reversible promoter. Suitable reversible promoters, including reversible inducible promoters are known in the art. Such reversible promoters may be isolated and derived from many organisms. Such reversible promoters may be isolated and derived from many organisms, e.g., eukaryotes and prokaryotes. Modification of reversible promoters derived from a first organism for use in a second organism is well known in the art Modification of reversible promoters derived from a first organism for use in a second organism, e.g, a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Thus, it will be appreciated that the present disclosure includes the use of any promoter/regulatory sequence capable of driving expression of the desired nuclease or RNA operably linked thereto.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′- and 3′-untranslated regions for mRNA stability and translation efficiency from highly-expressed genes like α-globin or β-globin; SV40 polyoma origins of replication and ColEl for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. Suitable vectors and methods for producing vectors containing transgenes are well known and available in the art. Selectable markers also include chloramphenicol resistance, tetracycline resistance, spectinomycin resistance, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into the cell, the vectors may be maintained as an autonomously replicating sequence or extrachromosomal element or may be integrated into host DNA.

The present composition and system (e.g., proteins, polynucleotides encoding these proteins, or compositions comprising the proteins and/or polynucleotides described herein) may be delivered by any suitable means. In certain embodiments, the composition or system is delivered in vivo. In other embodiments, the composition or system is delivered to isolated/cultured cells (e.g., autologous iPS cells) in vitro to provide modified cells useful for in vivo delivery to patients afflicted with a disease or condition.

Vectors according to the present disclosure can be transformed, transfected, or otherwise introduced into a wide variety of host cells. Transfection refers to the taking up of a vector by a host cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes the components of the present compositions and system is also within the scope of the present disclosure. Such a vector may be delivered into host cells by a suitable method. Methods of delivering vectors to cells are well known in the art and may include DNA or RNA electroporation, transfection reagents such as liposomes or nanoparticles to delivery DNA or RNA, delivery of DNA, RNA, or protein by mechanical deformation, or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. Additionally, delivery vehicles such as nanoparticle- and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, biolistics, and the like.

In some embodiments, a subject vector is a viral construct, e.g., a recombinant adeno-associated virus construct, a recombinant adenoviral construct, a recombinant lentiviral construct, a recombinant retroviral construct, etc. Suitable viral vectors include, but are not limited to, viral vectors based on vaccinia virus; poliovirus; adenovirus; adeno-associated virus, SV40; herpes simplex virus; human immunodeficiency virus; a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

In some embodiments a subject vector is an AAV vector. By adeno-associated virus, or “AAV” it is meant the virus itself or derivatives thereof. The term covers all subtypes and both naturally occurring and recombinant forms, except where required otherwise, for example, AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV type 11 (AAV-11), avian AAV, bovine AAV, canine AAV, equine AAV, primate AAV, non-primate AAV, ovine AAV, a hybrid AAV (i.e., an AAV comprising a capsid protein of one AAV subtype and genomic material of another subtype), an AAV comprising a mutant AAV capsid protein or a chimeric AAV capsid (i.e. a capsid protein with regions or domains or individual amino acids that are derived from two or more different serotypes of AAV, e.g., AAV-DJ, AAV-LK3, AAV-LK19). “Primate AAV” refers to AAV that infect primates, “non-primate AAV” refers to AAV that infect non-primate mammals, “bovine AAV” refers to AAV that infect bovine mammals, etc.

By a “recombinant AAV vector” or “rAAV vector” it is meant an AAV virus or AAV viral chromosomal material comprising a polynucleotide sequence not of AAV origin (e.g., a polynucleotide heterologous to AAV), typically a nucleic acid sequence of interest to be integrated into the cell following the subject methods. In general, the heterologous polynucleotide is flanked by at least one, and generally by two AAV inverted terminal repeat sequences (ITRs) In some instances, the recombinant viral vector also comprises viral genes important for the packaging of the recombinant viral vector material. Packaging refers to the series of intracellular events that result in the assembly and encapsulation of a viral particle, e.g., an AAV viral particle. Examples of nucleic acid sequences important for AAV packaging include the AAV “rep” and “cap” genes, which encode for replication and encapsulation proteins of adeno-associated virus, respectively. The term rAAV vector encompasses both rAAV vector particles and rAAV vector plasmids.

A “viral particle” refers to a single unit of virus comprising a capsid encapsulating a virus-based polynucleotide, e.g., the viral genome (as in a wild-type virus), or, e.g., the subject targeting vector (as in a recombinant virus). An AAV viral particle refers to a viral particle composed of at least one AAV capsid protein (typically by all of the capsid proteins of a wild-type AAV) and an encapsulated polynucleotide AAV vector. If the particle comprises a heterologous polynucleotide (e.g., a polynucleotide other than a wild-type AAV genome, such as a transgene to be delivered to a mammalian cell), it is typically referred to as an “rAAV vector particle” or simply an “rAAV vector.” Thus, production of rAAV particle necessarily includes production of rAAV vector, as such a vector is contained within an rAAV particle.

A rAAV virion can be constructed a variety of methods. For example, the heterologous sequence(s) can be directly inserted into an AAV genome which has had the major AAV open reading frames (“ORFs”) excised therefrom. Other portions of the AAV genome can also be deleted, so long as a sufficient portion of the ITRs remain to allow for replication and packaging functions. In order to produce rAAV virions, an AAV expression vector can be introduced into a suitable host cell using known techniques, such as by transfection. Particularly suitable transfection methods include calcium phosphate co-, direct micro-injection into cultured cells, electroporation, liposome mediated gene transfer, lipid-mediated transduction, and nucleic acid delivery using high-velocity microprojectiles. Suitable cells for producing rAAV virions include microorganisms, yeast cells, insect cells, and mammalian cells, that can be, or have been, used as recipients of a heterologous DNA molecule.

AAV virus that is produced may be replication competent or replication-incompetent. A “replication-competent” virus (e.g., a replication-competent AAV) refers to a phenotypically wild-type virus that is infectious and is also capable of being replicated in an infected cell (e.g., in the presence of a helper virus or helper virus functions). In the case of AAV, replication competence generally requires the presence of functional AAV packaging genes. In general, rAAV vectors as described herein are replication-incompetent in mammalian cells (especially in human cells) by virtue of the lack of one or more AAV packaging genes. Typically, such TAAV vectors lack any AAV packaging gene sequences in order to minimize the possibility that replication competent AAV are generated by recombination between AAV packaging genes and an incoming rAAV vector.

Retroviruses, for example, lentiviruses, are suitable for use in methods of the present disclosure. Commonly used retroviral vectors are unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog, and mouse; and xenotropic for most mammalian cell types except murine cells). The appropriate packaging cell line may be used to ensure that the cells are targeted by the packaged viral particles. Methods of introducing subject vector expression vectors into packaging cell lines and of collecting the viral particles that are generated by the packaging lines are well known in the art. Nucleic acids can also introduced by direct micro-injection (e.g., injection of RNA).

As noted elsewhere herein, proteins may instead be provided to cells as RNA (e.g., an RNA comprising the translational control element as discussed elsewhere herein). Methods of introducing RNA into cells may include, for example, direct injection, transfection, or any other method used for the introduction of DNA. The nuclease may also be introduced into a host cell directly as protein. In such instances, the nuclease may be delivered as an RNP (ribonucleoprotein complex) in which it is already complexed with an appropriate guide RNA.

The disclosed nucleic acids (e.g., vectors) and proteins can be delivered to cells using any convenient method. Suitable methods include, e.g., viral infection (e.g., AAV, adenovirus, lentiviral), transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like.

In some cases, a protein (e.g., engineered nuclease or nuclease) is delivered to a cell in a particle, or associated with a particle. In some cases, a nuclease is delivered with a cationic lipid and a hydrophilic polymer, for instance wherein the cationic lipid comprises 1,2-dioleoyl-3-trimethylammonium-propane (DOTAP) or 1,2-ditetradecanoyl-sn-glycero-3-phosphocholine (DMPC) and/or wherein the hydrophilic polymer comprises ethylene glycol or polyethylene glycol (PEG), and/or wherein the particle further comprises cholesterol.

A protein (e.g., engineered nuclease or nuclease) may be delivered using particles or lipid envelopes. For example, a biodegradable core-shell structured nanoparticle with a poly (β-amino ester) (PBAE) core enveloped by a phospholipid bilayer shell can be used. In some cases, particles/nanoparticles based on self-assembling bioadhesive polymers are used; such particles/nanoparticles may be applied to oral delivery of peptides, intravenous delivery of peptides and nasal delivery of peptides, e.g., to the brain. Other embodiments, such as oral absorption and ocular delivery of hydrophobic drugs are also contemplated. A molecular envelope technology, which involves an engineered polymer envelope which is protected and delivered to the desired cell, can be used.

Lipidoid compounds (e.g., as described in U.S. Patent Application Publication No. 2011/0293703) are also useful in the delivery of polynucleotides, and can be used to deliver the disclosed engineered nuclease or nucleases (or RNA or DNA encoding thereof). In one aspect, the aminoalcohol lipidoid compounds are combined with an agent to be delivered to a cell to form microparticles, nanoparticles, liposomes, or micelles. The aminoalcohol lipidoid compounds may be combined with other aminoalcohol lipidoid compounds, polymers (synthetic or natural), surfactants, cholesterol, carbohydrates, proteins, lipids, etc. to form the particles. These particles may then optionally be combined with a pharmaceutical excipient to form a pharmaceutical composition.

A poly(beta-amino alcohol) (PBAA) can be used to deliver a nuclease, or a nucleic acid encoding thereof, and gRNA, or a nucleic acid encoding thereof, to a target cell. U.S. Patent Application Publication No. 2013/0302401 relates to a class of poly(beta-amino alcohols) (PBAAs) that has been prepared using combinatorial polymerization.

Sugar-based particles, for example GalNAc, as described in International Patent Publication No. WO2014118272 (incorporated herein by reference in its entirety and Nair, J K et al., 2014, Journal of the American Chemical Society 136 (49), 16958-16961) can be used to deliver a nuclease, or a nucleic acid encoding thereof, and gRNA, or a nucleic acid encoding thereof, to a target cell.

In some cases, lipid nanoparticles (LNPs) are used to deliver a nuclease, or a nucleic acid encoding thereof, and gRNA, or a nucleic acid encoding thereof, to a target cell. Negatively charged polymers such as RNA may be loaded into LNPs at low pH values (e.g., pH 4) where the ionizable lipids display a positive charge. However, at physiological pH values, the LNPs exhibit a low surface charge compatible with longer circulation times. Four species of ionizable cationic lipids have been focused upon, namely 1,2-dilineoyl-3-dimethylammonium-propane (DLinDAP), 1,2-dilinoleyloxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminoethyl)-[1,3]-dioxolane (DLinKC2-DMA). Preparation of LNPs and is described in, e.g., Rosin et al. (2011) Molecular Therapy 19:1286-2200). The cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-O-[2″-(methoxypolyethyleneglycol 2000) succinoyl]-1,2-dimyristoyl-sn-glycol (PEG-S-DMG), and R-3-[(omega.-methoxy-poly(ethylene glycol)2000) carbamoyl]-1,2-dimyristyloxlpropyl-3-amine (PEG-C-DOMG) may be used. A nucleic acid may be encapsulated in LNPs containing DLinDAP, DLinDMA, DLinK-DMA, and DLinKC2-DMA (cationic lipid:DSPC:CHOL:PEGS-DMG or PEG-C-DOMG at 40:10:40:10 molar ratios). In some cases, 0.2% SP-DiOC18 is incorporated.

Spherical Nucleic Acid (SNA™) constructs and other nanoparticles (particularly gold nanoparticles) can be used to deliver a nuclease, or a nucleic acid encoding thereof, and gRNA, or a nucleic acid encoding thereof, to a target cell.

Self-assembling nanoparticles with RNA may be constructed with polyethyleneimine (PEI) that is PEGylated with an Arg-Gly-Asp (RGD) peptide ligand attached at the distal end of the polyethylene glycol (PEG).

Nanoparticles suitable for use in delivering a nuclease, or a nucleic acid encoding thereof, and gRNA, or a nucleic acid encoding thereof, to a target cell may be provided in different forms, e.g., as solid nanoparticles (e.g., metal such as silver, gold, iron, titanium), non-metal, lipid-based solids, polymers), suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically below 10 nm) that quantization of electronic energy levels occurs. Such nanoscale particles are used in biomedical applications as drug carriers or imaging agents and may be adapted for similar purposes in the present disclosure. In general, a “nanoparticle” refers to any particle having a diameter of less than 1000 nm. In some cases, nanoparticles suitable for use in delivering a nuclease or nucleic acid to a target cell have a diameter of 500 nm or less, e.g., from 25 nm to 35 nm, from 35 nm to 50 nm, from 50 nm to 75 nm, from 75 nm to 100 nm, from 100 nm to 150 nm, from 150 nm to 200 nm, from 200 nm to 300 nm, from 300 nm to 400 nm, or from 400 nm to 500 nm. In some cases, nanoparticles suitable for use in delivering a nuclease or nucleic acid to a target cell have a diameter of from 25 nm to 200 nm.

In some cases, an exosome is used to deliver a nuclease or engineered nuclease, or a nucleic acid encoding thereof, and gRNA, or a nucleic acid encoding thereof, to a target cell. Exosomes are endogenous nano-vesicles that transport RNAs and proteins, and which can deliver RNA to the brain and other target organs.

In some cases, a liposome is used to deliver a nuclease or engineered nuclease, or a nucleic acid encoding thereof, and gRNA, or a nucleic acid encoding thereof, to a target cell. Liposomes are spherical vesicle structures composed of a uni- or multi-lamellar lipid bilayer surrounding internal aqueous compartments and a relatively impermeable outer lipophilic phospholipid bilayer. Liposomes can be made from several different types of lipids; however, phospholipids are most commonly used to generate liposomes. Although liposome formation is spontaneous when a lipid film is mixed with an aqueous solution, it can also be expedited by applying force in the form of shaking by using a homogenizer, sonicator, or an extrusion apparatus Several other additives may be added to liposomes in order to modify their structure and properties. For instance, either cholesterol or sphingomyelin may be added to the liposomal mixture in order to help stabilize the liposomal structure and to prevent the leakage of the liposomal inner cargo. A liposome formulation may be mainly comprised of natural phospholipids and lipids such as 1,2-distearoryl-sn-glycero-3-phosphatidyl choline (DSPC), sphingomyelin, egg phosphatidylcholines and monosialoganglioside.

A stable nucleic-acid-lipid particle (SNALP) can be used to deliver a nuclease, or a nucleic acid encoding thereof, and gRNA, or a nucleic acid encoding thereof, to a target cell. The SNALP formulation may contain the lipids 3-N-[(methoxypoly(ethylene glycol) 2000) carbamoyl]-1,2-dimyristyloxy-propylamine (PEG-C-DMA), 1,2-dilinoleyloxy-N,N-dimethyl-3-aminopropane (DLinDMA), 1,2-distearoyl-sn-glycero-3-phosphocholine (DSPC) and cholesterol, in a 2:40:10:48 molar percent ratio. The SNALP liposomes may be prepared by formulating D-Lin-DMA and PEG-C-DMA with distearoylphosphatidylcholine (DSPC), Cholesterol and siRNA using a 25:1 lipid/siRNA ratio and a 48/40/10/2 molar ratio of Cholesterol/D-Lin-DMA/DSPC/PEG-C-DMA. The resulting SNALP liposomes can be about 80-100 nm in size. A SNALP may comprise synthetic cholesterol (Sigma-Aldrich, St Louis, Mo., USA), dipalmitoylphosphatidylcholine (Avanti Polar Lipids, Alabaster, Ala., USA), 3-N-[(w-methoxy poly(ethylene glycol)2000)carbamoyl]-1,2-dimyrestyloxypropylamine, and cationic 1,2-dilinoleyloxy-3-N,Ndimethylaminopropane. A SNALP may comprise synthetic cholesterol (Sigma-Aldrich), 1,2-distearoyl-sn-glycero-3-phosphocholine (DSPC; Avanti Polar Lipids Inc.), PEG-cDMA, and 1,2-dilinoleyloxy-3-(N;N-dimethyl)aminopropane (DLinDMA).

Other cationic lipids, such as amino lipid 2,2-dilinoleyl-4-dimethylaminoethyl-[1,3]-dioxolane (DLin-KC2-DMA) can be used to deliver a nuclease or nucleic acid to a target cell. A preformed vesicle with the following lipid composition may be contemplated: amino lipid, distearoylphosphatidylcholine (DSPC), cholesterol and (R)-2,3-bis(octadecyloxy) propyl-1-(methoxy poly(ethylene glycol)2000)propylcarbamate (PEG-lipid) in the molar ratio 40/10/40/10, respectively, and a FVII siRNA/total lipid ratio of approximately 0.05 (w/w). To ensure a narrow particle size distribution in the range of 70-90 nm and a low polydispersity index of 0.11.+−. 0.04 (n=56), the particles may be extruded up to three times through 80 nm membranes prior to adding the guide RNA. Particles containing the highly potent amino lipid 16 may be used, in which the molar ratio of the four lipid components 16, DSPC, cholesterol and PEG-lipid (50/10/38.5/1.5) which may be further optimized to enhance in vivo activity.

Lipids may be formulated with a nuclease or engineered nuclease, or a nucleic acid encoding thereof, and gRNA, or a nucleic acid encoding thereof, to form lipid nanoparticles (LNPs). Suitable lipids include, but are not limited to, DLin-KC2-DMA4, C12-200 and colipids disteroylphosphatidyl choline, cholesterol, and PEG-DMG may be formulated with a nuclease or nucleic acid using a spontaneous vesicle formation procedure.

A nuclease or engineered nuclease, or a nucleic acid encoding thereof, and gRNA, or a nucleic acid encoding thereof, may be delivered encapsulated in PLGA microspheres such as that further described in US published applications 20130252281 and 20130245107 and 20130244279.

Supercharged proteins can be used to deliver a nuclease or engineered nuclease, or a nucleic acid encoding thereof, and gRNA, or a nucleic acid encoding thereof, to a target cell. Supercharged proteins are a class of engineered or naturally occurring proteins with unusually high positive or negative net theoretical charge. Both supernegatively and superpositively charged proteins exhibit the ability to withstand thermally or chemically induced aggregation. Superpositively charged proteins are also able to penetrate mammalian cells. Associating cargo with these proteins, such as plasmid DNA, RNA, or other proteins, can facilitate the functional delivery of these macromolecules into mammalian cells both in vitro and in vivo.

Cell Penetrating Peptides (CPPs) can be used to deliver a nuclease or engineered nuclease, or a nucleic acid encoding thereof, and gRNA, or a nucleic acid encoding thereof, to a target cell. CPPs typically have an amino acid composition that either contains a high relative abundance of positively charged amino acids such as lysine or arginine or has sequences that contain an alternating pattern of polar/charged amino acids and non-polar, hydrophobic amino acids.

### Methods

The disclosure also provides methods of altering a target nucleic acid sequence (e.g., DNA or RNA). The phrase “altering a nucleic acid sequence,” as used herein, refers to modifying at least one physical feature of a nucleic acid sequence of interest. Nucleic acid alterations include, for example, single or double strand breaks, deletion, or insertion of one or more nucleotides, and other modifications that affect the structural integrity or nucleotide sequence of the nucleic acid sequence.

The methods comprise contacting a target nucleic acid sequence with a composition as disclosed herein, a system disclosed herein or a composition comprising the system. In some embodiments, system comprises a nuclease comprising an amino acid sequence having at least 70% identity, at least 80% identity, at least 90% identity, at least 95% identity or at least 99% identity to any of SEQ ID NOs: 1-23, 26-29, or 32-236 or a nucleic acid molecule comprising a sequence encoding the nuclease; and at least one guide RNA (gRNA) complementary to at least a portion of the target nucleic acid or a nucleic acid comprising a sequence encoding the at least one gRNA. In some embodiments, system comprises a nuclease comprising an amino acid sequence having about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or 100% identity to any of SEQ ID NOs: 1-23, 26-29, or 32-236 or a nucleic acid molecule comprising a sequence encoding the nuclease; and at least one guide RNA (gRNA) complementary to at least a portion of the target nucleic acid or a nucleic acid comprising a sequence encoding the at least one gRNA. In some embodiments, the system comprises an engineered nuclease, as disclosed herein, or a nucleic acid encoding thereof, and at least on gRNA complementary to at least a portion of the target nucleic acid or a nucleic acid comprising a sequence encoding the at least one gRNA.

In one embodiment, the method introduces a single strand or double strand break in the target nucleic acid sequence. In this respect, the disclosed systems may direct cleavage of one or both strands of a target DNA sequence, such as within the target genomic DNA sequence and/or within the complement of the target sequence.

In some embodiments, altering a DNA sequence comprises a deletion. The deletion may be upstream or downstream of the PAM binding side, so called unidirectional deletions. The deletion may encompass sequences on either side of the PAM binding site, a bidirectional deletion. The deletion of the DNA sequence may be of any size.

In some embodiments, contacting a target nucleic acid sequence comprises introducing the composition or system into the cell. As described above the composition or system may be introduced into eukaryotic or prokaryotic cells by methods known in the art. The cell may be a prokaryotic cell, a plant cell, an insect cell, a vertebrate cell, an invertebrate cell, an animal cell, a mammalian cell, or a human cell. In some embodiments, the cell is a plant cell. In some embodiments, the cell is an insect cell. In some embodiments, the cell is a vertebrate cell. In some embodiments, the cell is an invertebrate cell. In some embodiments, the cell is a mammalian cell. In some embodiments, the cell is a human cell. In some cases, the cell is ex vivo (e.g., fresh isolate-early passage). In some cases, the cell is in vivo. In some cases, the cell is in culture in vitro (e.g., immortalized cell line).

Cells may be from established cell lines or they may be primary cells, where “primary cells,” “primary cell lines,” and “primary cultures” are used interchangeably herein to refer to cells and cells cultures that have been derived from a subject and allowed to grow in vitro for a limited number of passages of the culture. For example, primary cultures are cultures that may have been passaged 0 times, 1 time, 2 times, 4 times, 5 times, 10 times, or 15 times, but not enough times go through the crisis stage. Typically, the primary cell lines are maintained for fewer than 10 passages in culture.

Suitable cells include, but are not limited to: bacterial cell; an archaeal cell; a eukaryotic cell; a cell of a single-cell eukaryotic organism; a plant cell; a protozoa cell; an algal cell, e.g., Botryococcus braunii, Chlamydomonas reinhardtii, Nannochloropsis gaditana, Chlorella pyrenoidosa, Sargassum patens, C. agardh, and the like; a fungal cell (e.g., a yeast cell); an animal cell; a cell from an invertebrate animal (e.g., fruit fly, a cnidarian, an echinoderm, a nematode, etc.); a cell of an insect (e.g., a mosquito, a bee; an agricultural pest; etc.); a cell of an arachnid (e.g., a spider; a tick; etc.); a cell of a vertebrate animal (e.g., a fish, an amphibian, a reptile, a bird, a mammal); a cell of a mammal (e.g., a cell of a rodent; a cell of a human; a cell of a non-human mammal; a cell of a rodent (e.g., a mouse, a rat); a cell of a lagomorph (e.g., a rabbit); a cell of an ungulate (e.g., a cow, a horse, a camel, a llama, a vicuña, a sheep, a goat, etc.); a cell of a marine mammal (e.g., a whale, a seal, an elephant seal, a dolphin, a sea lion; etc.) and the like. Any type of cell may be of interest (e.g., a stem cell, e.g., an embryonic stem (ES) cell, an induced pluripotent stem (iPS) cell, a germ cell (e.g., an oocyte, a sperm, an oogonia, a spermatogonia, etc.), an adult stem cell, a somatic cell, e.g., a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.). In some cases, the cell is a cell that does not originate from a natural organism (e g., the cell can be a synthetically made cell; also referred to as an artificial cell).

Non-limiting examples of plant cell include cells from: plant crops, fruits, vegetables, grains, soybean, corn, maize, wheat, seeds, tomatoes, rice, cassava, sugarcane, pumpkin, hay, potatoes, cotton, cannabis, tobacco, flowering plants, conifers, gymnosperms, angiosperms, ferns, clubmosses, hornworts, liverworts, mosses, dicotyledons, monocotyledons, seaweeds (e.g., kelp), and the like.

Suitable cells include a stem cell (e.g., an embryonic stem (ES) cell, an induced pluripotent stem (iPS) cell; a germ cell (e.g., an oocyte, a sperm, an oogonia, a spermatogonia, etc.); a somatic cell, e.g., a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some cases, the cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

In some cases, the cell is a stem cell. Stem cells include adult stem cells. Adult stem cells are also referred to as somatic stem cells.

Adult stem cells are resident in differentiated tissue but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals, and zoo, laboratory, sports, or pet animals, such as dogs, horses, cats, cows, mice, rats, rabbits, etc. In some cases, the stem cell is a human stem cell. In some cases, the stem cell is a rodent (e.g., a mouse; a rat) stem cell. In some cases, the stem cell is a non-human primate stem cell.

In some embodiments, the stem cell is a hematopoietic stem cell (HSC). HSCs are mesoderm-derived cells that can be isolated from bone marrow, blood, cord blood, fetal liver, and yolk sac. HSCs are characterized as CD34⁺ and CD3⁻. HSCs can repopulate the erythroid, neutrophil-macrophage, megakaryocyte, and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

In other embodiments, the stem cell is a neural stem cell (NSC). Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells, respectively. Methods of obtaining NSCs are known in the art.

In other embodiments, the stem cell is a mesenchymal stem cell (MSC). MSCs originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

In some embodiments, the cell is a T cell. The invention is not limited by the type of T cell. The T cells may be selected from, for example, CD3+ T cells, CD8+ T cells, CD4+ T cells, natural killer (NK) T cells, alpha beta T cells, gamma delta T cells, or any combination thereof (e.g., a combination of CD4+ and CD8+ T cells).

In some embodiments, the T cells are naturally occurring T cells. For example, the T cells may be isolated from a subject sample. In some embodiments, the T cell is an anti-tumor T cell (e.g., a T cell with activity against a tumor (e.g., an autologous tumor) that becomes activated and expands in response to antigen). Anti-tumor T cells include, but are not limited to, T cells obtained from resected tumors or tumor biopsies (e.g., tumor infiltrating lymphocytes (TILs)) and a polyclonal or monoclonal tumor-reactive T cell (e.g., obtained by apheresis, expanded ex vivo against tumor antigens presented by autologous or artificial antigen-presenting cells). In some embodiments, the T cells are expanded ex vivo.

A cell is in some cases a plant cell. A plant cell can be a cell of a monocotyledon. A plant cell can be a cell of a dicotyledon. The cells can be root cells, leaf cells, cells of the xylem, cells of the phloem, cells of the cambium, apical meristem cells, parenchyma cells, collenchyma cells, sclerenchyma cells, and the like. Plant cells include cells of agricultural crops such as wheat, corn, rice, sorghum, millet, soybean, etc. Plant cells include cells of agricultural fruit and nut plants, e.g., plant that produce apricots, oranges, lemons, apples, plums, pears, almonds, etc.

A plant cell can be a cell of a major agricultural plant, e.g., Barley, Beans (Dry Edible), Canola, Corn, Cotton (Pima), Cotton (Upland), Flaxseed, Hay (Alfalfa), Hay (Non-Alfalfa), Oats, Peanuts, Rice, Sorghum, Soybeans, Sugarbeets, Sugarcane, Sunflowers (Oil), Sunflowers (Non-Oil), Sweet Potatoes, Tobacco (Burley), Tobacco (Flue-cured), Tomatoes, Wheat (Durum), Wheat (Spring), Wheat (Winter), and the like. As another example, the cell is a cell of a vegetable crops which include but are not limited to, e.g., alfalfa sprouts, aloe leaves, arrow root, arrowhead, artichokes, asparagus, bamboo shoots, banana flowers, bean sprouts, beans, beet tops, beets, bittermelon, bok choy, broccoli, broccoli rabe (rappini), brussels sprouts, cabbage, cabbage sprouts, cactus leaf (nopales), calabaza, cardoon, carrots, cauliflower, celery, chayote, chinese artichoke (crosnes), chinese cabbage, chinese celery, chinese chives, choy sum, chrysanthemum leaves (tung ho), collard greens, corn stalks, corn-sweet, cucumbers, daikon, dandelion greens, dasheen, dau mue (pea tips), donqua (winter melon), eggplant, endive, escarole, fiddle head ferns, field cress, frisee, gai choy (chinese mustard), gailon, galanga (siam, thai ginger), garlic, ginger root, gobo, greens, hanover salad greens, huauzontle, jerusalem artichokes, jicama, kale greens, kohlrabi, lamb's quarters (quilete), lettuce (bibb), lettuce (boston), lettuce (boston red), lettuce (green leaf), lettuce (iceberg), lettuce (lolla rossa), lettuce (oak leaf-green), lettuce (oak leaf-red), lettuce (processed), lettuce (red leaf), lettuce (romaine), lettuce (ruby romaine), lettuce (russian red mustard), linkok, lo bok, long beans, lotus root, mache, maguey (agave) leaves, malanga, mesculin mix, mizuna, moap (smooth luffa), moo, moqua (fuzzy squash), mushrooms, mustard, nagaimo, okra, ong choy, onions green, opo (long squash), ornamental corn, ornamental gourds, parsley, parsnips, peas, peppers (bell type), peppers, pumpkins, radicchio, radish sprouts, radishes, rape greens, rape greens, rhubarb, romaine (baby red), rutabagas, salicornia (sea bean), sinqua (angled/ridged luffa), spinach, squash, straw bales, sugarcane, sweet potatoes, swiss chard, tamarindo, taro, taro leaf, taro shoots, tatsoi, tepeguaje (guaje), tindora, tomatillos, tomatoes, tomatoes (cherry), tomatoes (grape type), tomatoes (plum type), tumeric, turnip tops greens, turnips, water chestnuts, yampi, yams (names), yu choy, yuca (cassava), and the like.

A cell is in some cases an arthropod cell. For example, the cell can be a cell of a sub-order, a family, a sub-family, a group, a sub-group, or a species of, e.g., Chelicerata, Myriapodia, Hexipodia, Arachnida, Insecta, Archaeognatha, Thysanura, Palaeoptera, Ephemeroptera, Odonata, Anisoptera, Zygoptera, Neoptera, Exopterygota, Plecoptera, Embioptera, Orthoptera, Zoraptera, Dermaptera, Dictyoptera, Notoptera, Grylloblattidae, Mantophasmatidae, Phasmatodea, Blattaria, Isoptera, Mantodea, Parapneuroptera, Psocoptera, Thysanoptera, Phthiraptera, Hemiptera, Endopterygota or Holometabola, Hymenoptera, Coleoptera, Strepsiptera, Raphidioptera, Megaloptera, Neuroptera, Mecoptera, Siphonaptera, Diptera, Trichoptera, or Lepidoptera.

A cell is in some cases an insect cell. For example, in some cases, the cell is a cell of a mosquito, a grasshopper, a true bug, a fly, a flea, a bee, a wasp, an ant, a louse, a moth, or a beetle.

In some embodiments, the target nucleic acid is a nucleic acid endogenous to a target cell. In some embodiments, the target nucleic acid is a genomic DNA sequence. The term “genomic,” as used herein, refers to a nucleic acid sequence (e.g., a gene or locus) that is located on a chromosome in a cell.

In some embodiments, the target nucleic acid encodes a gene or gene product. The term “gene product,” as used herein, refers to any biochemical product resulting from expression of a gene Gene products may be RNA or protein. RNA gene products include non-coding RNA, such as tRNA, rRNA, micro RNA (miRNA), and small interfering RNA (siRNA), and coding RNA, such as messenger RNA (mRNA). In some embodiments, the target nucleic acid sequence encodes a protein or polypeptide.

The disclosed method may alter a target DNA sequence in a cell so as to modulate expression of the target DNA sequence, e.g., expression of the target DNA sequence is increased, decreased, or completely eliminated (e.g., via deletion of a gene). In one embodiment, the disclosed system cleaves a target DNA sequence of the host cell to produce double strand DNA breaks. The double strand breaks can be repaired by the host cell by either non-homologous end joining (NHEJ) or homologous recombination. In NHEJ, the double-strand breaks are repaired by direct ligation of the break ends to one another. In homologous recombination repair, a donor nucleic acid molecule comprising a second DNA sequence with homology to the cleaved target DNA sequence is used as a template for repair of the cleaved target DNA sequence, resulting in the transfer of genetic information from the donor nucleic acid molecule to the target DNA. As a result, new nucleic acid material is inserted/copied into the DNA break site. The modifications of the target sequence due to NHEJ and/or homologous recombination repair may lead to, for example, gene correction, gene replacement, gene tagging, transgene insertion, nucleotide deletion, gene disruption, gene mutation, gene knock-down, etc.

In some embodiments, the systems and methods described herein may be used to correct one or more defects or mutations in a gene (referred to as “gene correction”). In such cases, the target sequence encodes a defective version of a gene, and the disclosed compositions and systems further comprise a donor nucleic acid molecule which encodes a wild-type or corrected version of the gene.

In another embodiment, the method of altering a target sequence can be used to delete nucleic acids from a target sequence in a host cell by cleaving the target sequence and allowing the host cell to repair the cleaved sequence in the absence of an exogenously provided donor nucleic acid molecule. Deletion of a nucleic acid sequence in this manner can be used in a variety of applications, such as, for example, to remove disease-causing trinucleotide repeat sequences in neurons, to create gene knock-outs or knock-downs, and to generate mutations for disease models in research.

In some embodiments, the systems and methods described herein may be used to insert a gene or fragment thereof into a cell. In particular embodiments, the disclosed systems may be used to generate a cell that expresses a recombinant receptor In some embodiments, the recombinant receptor is a T cell receptor (TCR) or a chimeric antigen receptor (CAR). Also provided herein are cells, e.g., a T cell, comprising a recombinant receptor and/or a nucleic acid encoding thereof and a system (e.g., nuclease and at least one gRNA) as described herein.

In some embodiments, the system and methods described herein may be used to genetically modify a plant or plant cell. As used herein, genetically modified plants include a plant into which has been introduced an exogenous polynucleotide. Genetically modified plants also include a plant that has been genetically manipulated such that endogenous nucleotides have been altered to include a mutation, such as a deletion, an insertion, a transition, a transversion, or a combination thereof. For instance, an endogenous coding region could be deleted. Such mutations may result in a polypeptide having a different amino acid sequence than was encoded by the endogenous polynucleotide. Another example of a genetically modified plant is one having an altered regulatory sequence, such as a promoter, to result in increased or decreased expression of an operably linked endogenous coding region. The genetically modified plant may promote a desired phenotypic or genotypic plant trait.

Genetically modified plants can potentially have improved crop yields, enhanced nutritional value, and increased shelf life. They can also be resistant to unfavorable environmental conditions, insects, and pesticides. The present systems and methods have broad applications in gene discovery and validation, mutational and cisgenic breeding, and hybrid breeding. The present systems and methods may facilitate the production of a new generation of genetically modified crops with various improved agronomic traits such as herbicide resistance, herbicide tolerance, drought tolerance, male sterility, insect resistance, abiotic stress tolerance, modified fatty acid metabolism, modified carbohydrate metabolism, modified seed yield, modified oil percent, modified protein percent, resistance to bacterial disease, disease (e.g., bacterial, fungal, and viral) resistance, high yield, and superior quality. The present systems and methods may also facilitate the production of a new generation of genetically modified crops with optimized fragrance, nutritional value, shelf-life, pigmentations (e.g., lycopene content), starch content (e.g., low-gluten wheat), toxin levels, propagation and/or breeding and growth time. See, for example, CRISPR/Cas Genome Editing and Precision Plant Breeding in Agriculture (Chen et al., Annu Rev Plant Biol. 2019 Apr. 29; 70:667-69), incorporated herein by reference.

The present system and method may confer one or more of the following traits to the plant cell: herbicide tolerance, drought tolerance, male sterility, insect resistance, abiotic stress tolerance, modified fatty acid metabolism, modified carbohydrate metabolism, modified seed yield, modified oil percent, modified protein percent, resistance to bacterial disease, resistance to fungal disease, and resistance to viral disease.

The present disclosure provides for a modified plant cell produced by the present system and method, a plant comprising the plant cell, and a seed, fruit, plant part, or propagation material of the plant. Transformed or genetically modified plant cells of the present disclosure may be as populations of cells, or as a tissue, seed, whole plant, stem, fruit, leaf, root, flower, stem, tuber, grain, animal feed, a field of plants, and the like. The present disclosure provides a transgenic plant. The transgenic plant may be homozygous or heterozygous for the genetic modification. Also provided by the present disclosure are transformed or genetically modified plant cells, tissues, plants, and products that contain the transformed or genetically modified plant cells. The present disclosure further encompasses the progeny, clones, cell lines or cells of the transgenic plants.

The present system and method may be used to modify a plant stem cell. The present disclosure further provides progeny of a genetically modified cell, where the progeny can comprise the same genetic modification as the genetically modified cell from which it was derived. The present disclosure further provides a composition comprising a genetically modified cell.

In one embodiment, the transformed or genetically modified cells, and tissues and products comprise a nucleic acid integrated into the genome, and production by plant cells of a gene product due to the transformation or genetic modification.

Methods of introducing exogenous nucleic acids into plant cells are well known in the art. Such plant cells are considered “transformed.” DNA constructs can be introduced into plant cells by various methods, including, but not limited to PEG- or electroporation-mediated protoplast transformation, tissue culture or plant tissue transformation by biolistic bombardment, or the Agrobacterium-mediated transient and stable transformation. The transformation can be transient or stable transformation. Suitable methods also include viral infection (such as double stranded DNA viruses), transfection, conjugation, protoplast fusion, electroporation, particle gun technology, calcium phosphate precipitation, direct microinjection, silicon carbide whiskers technology, Agrobacterium-mediated transformation, and the like. The choice of method is generally dependent on the type of cell being transformed and the circumstances under which the transformation is taking place (i.e., in vitro, ex vivo, or in vivo). Transformation methods based upon the soil bacterium Agrobacterium tumefaciens are useful for introducing an exogenous nucleic acid molecule into a vascular plant. The wild-type form of Agrobacterium contains a Ti (tumor-inducing) plasmid that directs production of tumorigenic crown gall growth on host plants. Transfer of the tumor-inducing T-DNA region of the Ti plasmid to a plant genome requires the Ti plasmid-encoded virulence genes as well as T-DNA borders, which are a set of direct DNA repeats that delineate the region to be transferred. An Agrobacterium-based vector is a modified form of a Ti plasmid, in which the tumor inducing functions are replaced by the nucleic acid sequence of interest to be introduced into the plant host.

Agrobacterium-mediated transformation generally employs cointegrate vectors or binary vector systems, in which the components of the Ti plasmid are divided between a helper vector, which resides permanently in the Agrobacterium host and carries the virulence genes, and a shuttle vector, which contains the gene of interest bounded by T-DNA sequences. A variety of binary vectors are well known in the art and are commercially available, for example, from Clontech (Palo Alto, Calif.). Methods of coculturing Agrobacterium with cultured plant cells or wounded tissue such as leaf tissue, root explants, hypocotyledons, stem pieces or tubers, for example, also are well known in the art. See., e.g., Glick and Thompson, (eds.), Methods in Plant Molecular Biology and Biotechnology, Boca Raton, Fla.: CRC Press (1993), incorporated herein by reference.

Microprojectile-mediated transformation also can be used to produce a transgenic plant. This method, first described by Klein et al. (Nature 327:70-73 (1987), incorporated herein by reference), relies on microprojectiles such as gold or tungsten that are coated with the desired nucleic acid molecule by precipitation with calcium chloride, spermidine, or polyethylene glycol. The microprojectile particles are accelerated at high speed into an angiosperm tissue using a device such as the BIOLISTIC PD-1000 (Biorad; Hercules Calif.).

In one embodiment, the present systems and methods may be adapted to use in plants. In one embodiment, a series of plant-specific RNA-guided Genome Editing vectors (pRGE plasmids) are provided for expression of the present system in plants. The vectors may be optimized for transient expression of the present system in plant protoplasts, or for stable integration and expression in intact plants via the Agrobacterium-mediated transformation. In one aspect, the vector constructs include a nucleotide sequence comprising a DNA-dependent RNA polymerase III promoter, wherein the promoter is operably linked to a gRNA molecule and a Pol III terminator sequence, and a nucleotide sequence comprising a DNA-dependent RNA polymerase II promoter operably linked to a nucleic acid sequence encoding the nuclease.

In certain embodiments, the present systems and methods use a monocot promoter to drive the expression of one or more components of the present systems (e.g., gRNA) in a monocot plant. In certain embodiments, the present systems and methods use a dicot promoter to drive the expression of one or more components of the present systems (e.g., gRNA) in a dicot plant. In some embodiments, the present system is transiently expressed in plant protoplasts. Vectors for transient transformation of plants include, but are not limited to, pRGE3, pRGE6, pRGE31, and pRGE32. In some embodiment, the vector may be optimized for use in a particular plant type or species, such as pStGE3.

In one embodiment, the present system may be stably integrated into the plant genome, for example via Agrobacterium-mediated transformation. Thereafter, one or more components of the present system (e.g., the transgene) may be removed by genetic cross and segregation, which may lead to the production of non-transgenic, but genetically modified plants or crops. In one embodiment, the vector is optimized for Agrobacterium-mediated transformation. In one embodiment, the vector for stable integration is pRGEB3, pRGEB6, pRGEB31, pRGEB32, or pStGEB3.

The present system may be used in various bacterial hosts, including human pathogens that are medically important, and bacterial pests that are key targets within the agricultural industry, as well as antibiotic resistant versions thereof.

The system and method may be designed to target any gene or any set of genes, such as virulence or metabolic genes, for clinical and industrial applications in other embodiments. For example, the present systems and methods may be used to target and eliminate virulence genes from the population, to perform in situ gene knockouts, or to stably introduce new genetic elements to the metagenomic pool of a microbiome. The present systems and methods may be used to treat a multi-drug resistance bacterial infection in a subject. The present systems and methods may be used for genomic engineering within complex bacterial consortia.

The present systems and methods may be used to inactivate microbial genes. In some embodiments, the gene is an antibiotic resistance gene. For example, the coding sequence of bacterial resistance genes may be disrupted in vivo by insertion of a DNA sequence, leading to non-selective re-sensitization to drug treatment.

In some embodiments, introducing the system into a cell comprises administering the system to a subject. In some embodiments, the subject is human. The administering may comprise in vivo administration. In alternative embodiments, a vector is contacted with a cell in vitro or ex vivo and the treated cell, containing the system, is transplanted into a subject.

The components of the composition, system or ex vivo treated cells may be administered to a cell or subject with a pharmaceutically acceptable carrier or excipient as a pharmaceutical composition. In some embodiments, the components of the present system may be mixed, individually or in any combination, with a pharmaceutically acceptable carrier to form pharmaceutical compositions, which are also within the scope of the present disclosure.

The methods described here also provide for treating a disease or condition in a subject. The method may comprise administering to the subject, in vivo, or by transplantation of ex vivo treated cells (e.g., disclosed T cells), a therapeutically effective amount of the present system, or components thereof. A “subject” or “patient” may be human or non-human and may include, for example, animal strains or species used as “model systems” for research purposes, such a mouse model as described herein.

In some embodiments, the systems and methods are used to treat a pathogen or parasite on or in a subject by altering the pathogen or parasite. In some embodiments, the systems and methods target a “disease-associated” gene. The term “disease-associated gene,” refers to any gene or polynucleotide whose gene products are expressed at an abnormal level or in an abnormal form in cells obtained from a disease-affected individual as compared with tissues or cells obtained from an individual not affected by the disease. A disease-associated gene may be expressed at an abnormally high level or at an abnormally low level, where the altered expression correlates with the occurrence and/or progression of the disease. A disease-associated gene also refers to a gene, the mutation or genetic variation of which is directly responsible or is in linkage disequilibrium with a gene(s) that is responsible for the etiology of a disease. Examples of genes responsible for such “single gene” or “monogenic” diseases include, but are not limited to, adenosine deaminase, α-1 antitrypsin, cystic fibrosis transmembrane conductance regulator (CFTR), β-hemoglobin (HBB), oculocutaneous albinism II (OCA2), Huntingtin (HTT), dystrophia myotonica-protein kinase (DMPK), low-density lipoprotein receptor (LDLR), apolipoprotein B (APOB), neurofibromin I (NF1), polycystic kidney disease 1 (PKD1), polycystic kidney disease 2 (PKD2), coagulation factor VIII (F8), dystrophin (DMD), phosphate-regulating endopeptidase homologue, X-linked (PHEX), methyl-CpG-binding protein 2 (MECP2), and ubiquitin-specific peptidase 9Y, Y-linked (USP9Y). Other single gene or monogenic diseases are known in the art and described in, e.g., Chial, H. Rare Genetic Disorders: Learning About Genetic Disease Through Gene Mapping, SNPs, and Microarray Data, Nature Education 1(1).192 (2008); Online Mendelian Inheritance in Man (OMIM); and the Human Gene Mutation Database (HGMD). In another embodiment, the target genomic DNA sequence can comprise a gene, the mutation of which contributes to a particular disease in combination with mutations in other genes. Diseases caused by the contribution of multiple genes which lack simple (i.e., Mendelian) inheritance patterns are referred to in the art as a “multifactorial” or “polygenic” disease. Examples of multifactorial or polygenic diseases include, but are not limited to, asthma, diabetes, epilepsy, hypertension, bipolar disorder, and schizophrenia. Certain developmental abnormalities also can be inherited in a multifactorial or polygenic pattern and include, for example, cleft lip/palate, congenital heart defects, and neural tube defects. In another embodiment, the target DNA sequence can comprise a cancer oncogene.

The present disclosure provides for gene editing methods that can ablate a disease-associated gene (e.g., a cancer oncogene), which in turn can be used for in vivo gene therapy for patients. In some embodiments, the gene editing methods include donor nucleic acids comprising therapeutic genes.

In some embodiments, an effective amount of the components of the present system or compositions as described herein can be administered. Within the context of the present disclosure, the term “effective amount” refers to that quantity of the components of the system such that modification of the target nucleic acid is achieved.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

A wide range of additional therapies may be used in conjunction with the methods of the present disclosure. The additional therapy may be administration of an additional therapeutic agent or may be an additional therapy not connected to administration of another agent. Such additional therapies include, but are not limited to, surgery, immunotherapy, radiotherapy. The additional therapy may be administered at the same time as the above methods. In some embodiments, the additional therapy may precede or follow the treatment of the disclosed methods by time intervals ranging from hours to months.

In some embodiments, a therapeutically effective amount of a system or composition as described herein, is administered alone or in combination with a therapeutically effective amount of at least one additional therapeutic agent. In some embodiments, effective combination therapy is achieved with a single composition or pharmacological formulation or with two distinct compositions or formulations, administered at the same time or separated by a time interval. The at least one additional therapeutic agent may comprise any manner of therapeutic, including protein, small molecule, nucleic acids, and the like. For example, exemplary additional therapeutic agents include, but are not limited to, immune modulators, chemotherapeutic agents, a nucleic acid (e.g., mRNA, aptamers, antisense oligonucleotides, ribozyme nucleic acids, interfering RNAs, antigene nucleic acids), decongestants, steroids, analgesics, antimicrobial agents, immunotherapies, or any combination thereof.

In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (e.g., the period prior to clinical manifestation of a disease) and/or reduce the risk of developing or worsening a disease. For example, in connection with cancer the term “treat” may mean eliminate or reduce a patient's tumor burden, or prevent, delay, or inhibit metastasis, etc.

The phrase “pharmaceutically acceptable,” as used in connection with compositions and/or cells of the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

In some cases, desirable delivery systems provide for roughly uniform distribution and have controllable rates of release of their components (e.g., vectors, proteins, nucleic acids) in vivo. A variety of different media are described below that are useful in creating composition delivery systems. It is not intended that any one medium is limiting to the present invention. Note that any medium may be combined with another medium or carrier; for example, in one embodiment a polymer microparticle attached to a compound may be combined with a gel medium. An implantable device can be used to deliver a nuclease, or a nucleic acid encoding thereof, and gRNA, or a nucleic acid encoding thereof, to, for example, a target cell in vivo.

Carriers or mediums contemplated include materials such as gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof.

In some cases, a carrier/medium can include a microparticle. Microparticles can include, but are not limited to, liposomes, nanoparticles, microspheres, nanospheres, microcapsules, and nanocapsules. In some cases, microparticle can include one or more of the following: a poly(lactide-co-glycolide), aliphatic polyesters including, but not limited to, poly-glycolic acid and poly-lactic acid, hyaluronic acid, modified polysaccharides, chitosan, cellulose, dextran, polyurethanes, polyacrylic acids, pseudo-poly(amino acids), polyhydroxybutyrate-related copolymers, polyanhydrides, polymethylmethacrylate, poly(ethylene oxide), lecithin and phospholipids-in any combination thereof.

In some cases, a carrier/medium can include a liposome that is capable of attaching and releasing therapeutic agents (e.g., the subject nucleic acids and/or proteins). Liposomes are microscopic spherical lipid bilayers surrounding an aqueous core that are made from amphiphilic molecules such as phospholipids. For example, a liposome may trap a therapeutic agent between the hydrophobic tails of the phospholipid micelle. Water soluble agents can be entrapped in the core and lipid-soluble agents can be dissolved in the shell-like bilayer. Liposomes have a special characteristic in that they enable water soluble and water insoluble chemicals to be used together in a medium without the use of surfactants or other emulsifiers. Liposomes can form spontaneously by forcefully mixing phospholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life.

In some embodiments, a cationic or anionic liposome is used as part of a subject composition or method, or liposomes having neutral lipids can also be used. Cationic liposomes can include negatively-charged materials by mixing the materials and fatty acid liposomal components and allowing them to charge-associate. The choice of a cationic or anionic liposome depends upon the desired pH of the final liposome mixture.

Any element of any suitable CRISPR/Cas gene editing system known in the art can be employed in the systems and methods described herein, as appropriate. CRISPR/Cas gene editing technology is described in detail in, for example, U.S. Pat. Nos. 8,546,553, 8,697,359; 8,771,945; 8,795,965; 8,865,406; 8,871,445; 8,889,356; 8,889,418; 8,895,308; 8,9066,616; 8,932,814; 8,945,839; 8,993,233; 8,999,641; 9,115,348; 9,149,049; 9,493,844; 9,567,603; 9,637,739; 9,663,782; 9,404,098; 9,885,026; 9,951,342; 10,087,431; 10,227,610; 10,266,850, 10,601,748; 10,604,771; and 10,760,064; and U.S. Patent Application Publication Nos. US2010/0076057; US2014/0113376; US2015/0050699; US2015/0031134; US2014/0357530, US2014/0349400; US2014/0315985; US2014/0310830; US2014/0310828; US2014/0309487; US2014/0294773; US2014/0287938; US2014/0273230; US2014/0242699; US2014/0242664, US2014/0212869; US2014/0201857; US2014/0199767; US2014/0189896; US2014/0186919; US2014/0186843; and US2014/0179770, each incorporated herein by reference.

### Kits

Also within the scope of the present disclosure are kits that include the compositions, systems, or components thereof as disclosed herein.

For example the kits may contain one or more reagents or other components useful, necessary, or sufficient for practicing any of the methods described herein, such as, CRISPR reagents (engineered nuclease, a nuclease comprising an amino acid sequence having at least 70% identity, at least 80% identity, at least 90% identity, at least 95% identity or at least 99% identity to any of SEQ ID NOs: 1-23, 26-29, or 32-236, guide RNAs, vectors, compositions, etc.), transfection or administration reagents, negative and positive control samples (e.g., cells, template DNA), cells, containers housing one or more components (e.g., microcentrifuge tubes, boxes), detectable labels, detection and analysis instruments, software, instructions, and the like.

The kit may include instructions for use in any of the methods described herein. The instructions can comprise a description of administration of the present system or composition to a subject to achieve the intended effect. The instructions generally include information as to dosage, dosing schedule, and route of administration for the intended treatment. The kit may further comprise a description of selecting a subject suitable for treatment based on identifying whether the subject is in need of the treatment.

The kits provided herein are in suitable packaging. Suitable packaging includes, but is not limited to, vials, bottles, jars, flexible packaging, and the like. A kit may have a sterile access port (for example, the container may be an intravenous solution bag or a vial having a stopper pierceable by a hypodermic injection needle). The container may also have a sterile access port.

The packaging may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

The kit may further comprise a device for holding or administering the present system or composition. The device may include an infusion device, an intravenous solution bag, a hypodermic needle, a vial, and/or a syringe.

The kit will typically be provided with its various components in one or more packages, e.g., a fiber-based, a cardboard, polymeric, or a Styrofoam box. The enclosure(s) can be configured so as to maintain a temperature differential between the interior and the exterior, for example, to provide insulating properties to keep the reagents at a preselected temperature for a preselected time. The packaging can be air-tight, waterproof (e.g., impermeable to changes in moisture or evaporation), and/or light-tight.

### EXAMPLES

The following are examples of the present invention and are not to be construed as limiting.

### Example 1

### Nuclease and Guide RNA Vectors

Nuclease expression vectors Codon-optimized genes encoding the candidate nucleases (nuclease amino acid sequences SEQ ID NOs: 1-23) were synthesized and cloned into a mammalian expression vector under the CMV promoter. All sequences included a Nucleoplasmin Nuclear Localization Sequence (NLS) on their C-terminal, followed by a 3× HA tag.

crRNA vectors To make specific guide vectors for each nuclease, Short (mature crRNA) or Long (pre-crRNA) direct repeat sequences specific for each of the nucleases were placed downstream of the U6 promoter with either a starting G or starting A. Table 1 provides the Short and Long direct repeat sequences for each nuclease. For editing to occur with the nucleases, a functional crRNA sequence is placed upstream of the spacer target sequence.

TABLE 1
crRNA sequences for exemplary nucleases

| Nuclease ID | Long | Short |
|---|---|---|
| NUX012 | GTTTAATAAGAATACATAATTTCTAC | AATTTCTACTATCGTAGAT |
| | TATCGTAGAT (SEQ ID NO: 418) | (SEQ ID NO: 237) |
| NUX014 | GACTAAAACCCCCAACAAATTTCTA | AATTTCTACTTTTGTAGAT |
| | CTTTTGTAGAT (SEQ ID NO: 238) | (SEQ ID NO: 239) |
| NUX015 | ATTTAAACATAATATCTAATTICTAC | AATTTCTACTTTTGTAGAT |
| | TTTTGTAGAT (SEQ ID NO: 240) | (SEQ ID NO: 241) |
| NUX016 | GTTTAATACCTATTTAAAATTTCTAC | AATTTCTACTATTGTAGAT |
| | TATTGTAGAT (SEQ ID NO: 242) | (SEQ ID NO: 243) |
| NUX017 | GTTTAATAACCCTATATAATTTCTAC | AATTTCTACTATTGTAGAT |
| | TATTGTAGAT (SEQ ID NO: 244) | (SEQ ID NO: 245) |
| NUX018 | GGCTATACGCCTACGAAAATTTCTA | AATTTCTACTCTTGTAGAT |
| | CTCTTGTAGAT (SEQ ID NO: 246) | (SEQ ID NO: 247) |
| NUX019 | GGCTAACAGCCTTTTAAATTTCTACT | AATTTCTACTGTGTGTAGAT |
| | GTGTGTAGAT (SEQ ID NO: 248) | (SEQ ID NO: 249) |
| NUX021 | CTTAATAGCTGATATAAATTTCTACT | AATTTCTACTGTTGTAGAT |
| | GTTGTAGAT (SEQ ID NO: 250) | (SEQ ID NO: 251) |
| NUX022 | GTTTATTCCTTATAGTAGATTICTAC | GATTTCTACTTTTGTAGAT |
| | TTTTGTAGAT (SEQ ID NO: 252) | (SEQ ID NO: 253) |
| NUX023 | GTTTAATGCCTTATATAAATTICTAC | AATTTCTACTATTGTAGAT |
| | TATTGTAGAT (SEQ ID NO: 254) | (SEQ ID NO: 255) |
| NUX024 | GTTTAATACCTTATATAAATTICTAC | AATTTCTACTATTGTAGAT |
| | TATTGTAGAT (SEQ ID NO: 256) | (SEQ ID NO: 257) |
| NUX025 | GTTTAATACCTTATATAAATTTCTAC | AATTTCTACTATTGTAGAT |
| | TATTGTAGAT (SEQ ID NO: 258) | (SEQ ID NO: 259) |
| NUX026 | GTCTAGGACAATATGAAAATTTCTA | AATTTCTACTATTGTAGAT |
| | CTATTGTAGAT (SEQ ID NO: 260) | (SEQ ID NO: 261) |
| NUX027 | GTCTAATTTTTACGGTCAATTTCTAC | AATTTCTACTAATGTAGAT |
| | TAATGTAGAT (SEQ ID NO: 262) | (SEQ ID NO: 263) |
| NUX030 | GTCTAAAGGTACCACCAAATTTCTA | AATTTCTACTGTTGTAGAT |
| | CTGTTGTAGAT (SEQ ID NO: 264) | (SEQ ID NO: 265) |
| NUX031 | GATTAATAATCCCTAATAATTTCTAC | AATTTCTACTGTTGTAGAT |
| | TGTTGTAGAT (SEQ ID NO: 266) | (SEQ ID NO: 267) |
| NUX032 | GACTAATAAGCCAAATGAATTTCTA | AATTTCTACTATTGTAGAT |
| | CTATTGTAGAT (SEQ ID NO: 268) | (SEQ ID NO: 269) |
| NUX033 | GGCTATAGGCCTTTTATAATTTCTAC | AATTTCTACTATTGTAGAT |
| | TATTGTAGAT (SEQ ID NO: 270) | (SEQ ID NO: 271) |
| NUX034 | GTTTAATACCTTATATAAATTTCTAC | AATTTCTACTATTGTAGAT |
| | TATTGTAGAT (SEQ ID NO: 272) | (SEQ ID NO: 273) |
| NUX035 | GCCTAATAAGGTAATGTAATTTCTA | AATTTCTACTATTGTAGAT |
| | CTATTGTAGAT (SEQ ID NO: 274) | (SEQ ID NO: 275) |
| NUX036 | GGCTAACAGCCTTTTAAATTTCTACT | AATTTCTACTGTGTGTAGAT |
| | GTGTGTAGAT (SEQ ID NO: 276) | (SEQ ID NO: 277) |
| NUX037 | ATAGGAACAGGTATCCTAAACGGAT | |
| | GCTGATGTGAC (SEQ ID NO: 278) | |
| NUX038 | GTGCTGTTGATCTTCACAAGGGGAT | |
| | CAATAAGGCAC (SEQ ID NO: 279) | |
| NUX039 | GATTAATAATCCCTAATAATTTCTAC | AATTTCTACTGTTGTAGAT |
| | TGTTGTAGAT (SEQ ID NO: 280) | (SEQ ID NO: 281) |
| NUX040 | GATTAATAATCCCTAATAATTTCTAC | AATTTCTACTGTTGTAGAT |
| | TGTTGTAGAT (SEQ ID NO: 282) | (SEQ ID NO: 283) |
| NUX041 | GATTAATAATCCCTAATAATTTCTAC | AATTTCTACTGTTGTAGAT |
| | TGTTGTAGAT (SEQ ID NO: 284) | (SEQ ID NO: 285) |
| NUX043 | GATTAATAATCCCTAATAATTTCTAC | |
| | TGTTGTAGAT (SEQ ID NO: 286) | |
| NUX047 | GTTTATAACTTATAAAAAATTTCTAC | AATTTCTACTATTGTAGAT |
| | TATTGTAGAT (SEQ ID NO: 287) | (SEQ ID NO: 288) |

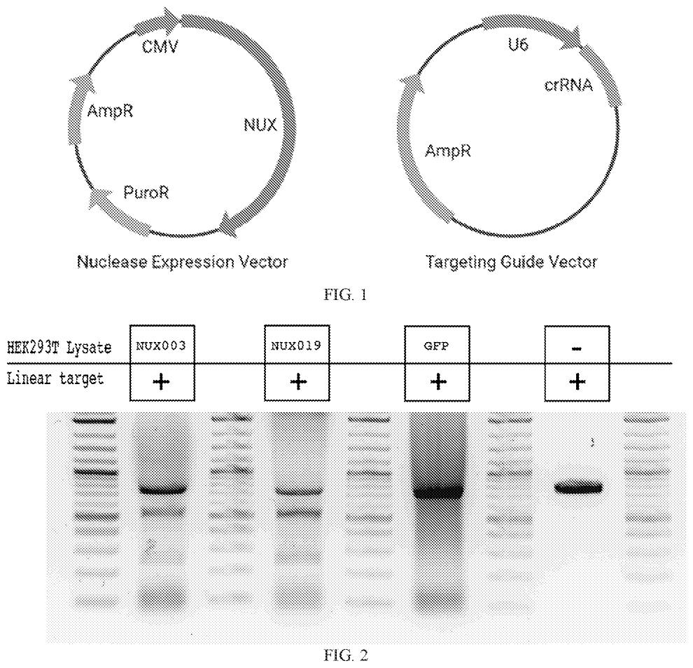

Exemplary vectors for the NUX019 expression vector and the corresponding guide vector with a NUX019 Short (pre-crRNA) sequence with a BbsI cloning cassette are shown in FIG. 1.

Targeting guide vectors To make the targeting guides, the crRNA vectors were digested with BbsI enzyme following the vendor's protocol and ligated to a 1:200 dilution of forward and reverse annealed oligos for each target. These oligonucleotide sequences (shown in Table 2) contained an overhang that matches the direct repeat and the terminator sequence of the vector.

TABLE 2
Oligonucleotides used to ligate targets
into crRNA vectors

| Target | Forward | Reverse |
|---|---|---|
| DNMT1-3 | AGATCTGATGGT | AGAATAACAGA |
| | CCATGTCTGTTA | CATGGACCATC |
| | (SEQ ID NO: 289) | AG |
| | | (SEQ ID NO: 290) |
| FANCF-1 | AGATGGCGGGG | AGAATCCCGGA |
| | TCCAGTTCCGG | ACTGGACCCCG |
| | GA | CC |
| | (SEQ ID NO: 291) | (SEQ ID NO: 292) |
| FANCF-2 | AGATGTCGGCA | AGAAGCGAATG |
| | TGGCCCCATTC | GGGCCATGCC |
| | GC | GAC |
| | (SEQ ID NO: 293) | (SEQ ID NO: 294) |
| SMN2- | AGATCAAAAGT | AGAATATGAAA |
| CR3L | AAGATTCACTTT | GTGAATCTTAC |
| | CATA | TTTTG |
| | (SEQ ID NO: 295) | (SEQ ID NO: 296) |
| GRIN2B | AGATGTGCTCA | AGAACCTTATC |
| | ATGAAAGGAGA | TCCTTTCATTGA |
| | TAAGG | GCAC |
| | (SEQ ID NO: 297) | (SEQ ID NO: 298) |

### Example 2

### Nuclease Expression Assays

Hek293T cells were transfected with each HA tagged nuclease and the respective guide following the protocol of Example 3. 72 hours post transfection the transfected cells were harvested by trypsinization and a subsequent centrifugation at 30 0 rpm for 10 minutes. Each cell pellet was lysed using 1.5× cell pellet weight to volume of NP40 lysis buffer (50 mM HEPES-KOH PH 7.5, 150 mM KCl, 2 mM EDTA, 0.5% Nonidet P-40 alternative, 0.5 mM DTT, 1 Complete EDTA-free Protease Inhibitor Cocktail tablet per 50 mL of buffer). Cells were lysed by incubation with the lysis buffer for 10 minutes on ice before a 10 minute spin at 13000 rpm. The protein content of the supernatant was normalized across samples before loading using a Bradford (sigma-B6916) assay. Normalized samples were loaded onto a 4-20% Bis-Tris gel (GenScript M00657) and run for 75 minutes at 140 volts.

For western blot analysis, the samples were transferred using program PO (20 V for 1 min 23 V for 4 min 25 V for 2 min) on the iBlot 2 dry blotting system (IB21001). The resulting PVDF membranes were then blotted using the iBind system (SLF1000) and the following primary antibodies: anti-HA tag (MAB0601) at 1:1000; anti-HSP90 (4874S) at 1:1000. The western blots were developed using Hypersensitive ECL Chemiluminescent Substrate (AR170).

For Coomassie analysis, the gel was incubated in ultrapure water for 2 minutes before being microwaved for 30 seconds in 50 mLs of GelCode Blue Safe Protein stain (Thermo scientific-1860957). The gel was further incubated for 10 minutes while shaking in the Coomassie stain before being de-stained in ultrapure water overnight while shaking.

### Example 3

### Editing Activity Assessment in Human Cells

The nucleases were tested in HEK293T cells through plasmid transfection using Mirus Transit 2020 reagent. Using the vectors constructed in Example 1, tests were performed in 96 well plates transfected with 150 ng of nuclease expression vector with 50 ng of targeting guide vector following the Mirus Transit 2020 transfection recommendations. Samples were incubated for 72 h and harvested with Quick extract. Genomic DNA was normalized to 200 ng and amplified using genomic region specific primers (Table 3). Samples were checked on a 2% agarose gel for purity, cleaned up and sequenced by Sanger sequencing. 3 uL from the sample was saved and prepared for T7E1 assays. TIDE (Tracking of Indels by Decomposition) analysis was performed following the method of Brinkman et al., (Brinkman E K, Chen T, Amendola M, van Steensel B. Nucleic Acids Res. 2014; 42(22): e168, incorporated herein by reference in its entirety) and recommendations at tide(dot)nki(dot)nl. TIDE output data on editing efficiency was plotted using Prism software. The list of targets used to validate the nucleases is provided in Table 4.

TABLE 3
Amplification primers for the genomic
regions targeted by the nucleases

| Target | Forward primer | Reverse primer |
|---|---|---|
| DNMT1-3 | CCAGAATGCACA | GCCAAAGCCCG |
| | AAGTACTGCAC | AGAGAGTGCC |
| | (SEQ ID NO: 299) | (SEQ ID NO: 300) |
| FANCF-1 | GGAGACGTTCAT | CGCCTGGGTCT |
| | GACTGGCA | TCATCAGAG |
| | (SEQ ID NO: 301) | (SEQ ID NO: 302) |
| FANCF-2 | GGAGACGTTCAT | CGCCTGGGTC |
| | GACTGGCA | TTCATCAGAG |
| | (SEQ ID NO: 303) | (SEQ ID NO: 304) |
| SMN2- | CCTCCAAAAGTG | TTCTTCCACA |
| | CAAGGC (SEQ | CAACCAACCA (SEQ |
| CR3L | ID NO: 305) | ID NO: 306) |
| GRIN2B | GCATACTCGCAT | CTCCCTGCAG |
| | GGCTACCT (SEQ | CCCCTTTTTA (SEQ |
| | ID NO: 307) | ID NO: 308) |

TABLE 4
Targets used to validate editing
of nucleases with TTTV PAM

| Target | Sequence | PAM |
|---|---|---|
| DNMT1-3 | CTGATGGTCCATGTCTGTTA | TTTC |
| | (SEQ ID NO: 309) | |
| FANCF-1 | GGCGGGGTCCAGTTCCGGGA | TTTG |
| | (SEQ ID NO: 310) | |
| FANCF-2 | GTCGGCATGGCCCCATTCGC | TTTG |
| | (SEQ ID NO: 311) | |
| SMN2-CR3L | CAAAAGTAAGATTCACTTTCATA | TTTA |
| | (SEQ ID NO: 312) | |
| GRIN2B | GTGCTCAATGAAAGGAGATAAGG | TTTG |
| | (SEQ ID NO: 313) | |

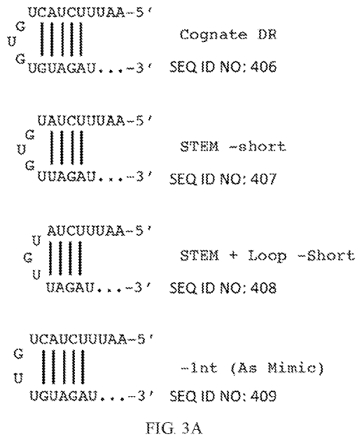

T7E1 Assay To assess the editing of the nucleases through T7E1 digestion, the target region of edited cells as well as wild type cells was amplified as described above. PCR amplicons were cleaned up and 200 ng of DNA was utilized for the T7E1 assay. Heteroduplex formation was performed by combining the pure DNA with water, Kapa GC buffer and 1 M KCl to a total of 10 μL. This reaction was then hybridized using the thermocycler with conditions: 95° C. for 10 minutes followed by slow decrease in temperature (2° C./see) until 25° C. was reached. Samples then are cooled down to 4° C. Digestion of the re-annealed DNA was performed by the addition of 5 units of T7 endonuclease I and NEB2 buffer to a total of 15 uL. Samples were then incubated at 37° C. for 30 minutes. To quench the samples, 5× loading dye with SDS was added and incubated for 10 minutes at 70° C. The reactions were then run on a 2% agarose gel at 120V for 30 minutes. An exemplary T7E1 assay with NUX0003, NUX019, and two no-nuclease controls are shown in FIG. 2. The presence of T7E1 cleavage products in the NUX003 and NUX019 conditions indicate indel formation at the target site in the genomic DNA due to CRISPR nuclease activity. The control conditions show no T7E1 cleavage products indicating no indel formation.

Additional nucleases were assayed for the ability to edit the FANCF2 target GTCGGCATGGCCCCATTCGC (SEQ ID NO: 311) using the T7E1 assay. Primers used for amplification were forward: GTCTCCAAGGTGAAAGCGGA (SEQ ID NO: 314) and reverse: GCGACAAAAGGCAGCAAAGA (SEQ ID NO: 315). AsCas12a was used as a positive control and the absence of nuclease as a negative control. Nuclease activity was confirmed for NUX012, NUX016, NUX017, NUX019, NUX021, NUX025, NUX026, NUX030, NUX031, NUX032, NUX034, NUX035 based upon the presence of T7E1 cleavage products.

### Example 4

crRNA Stem-Loop Modifications

An exploration of crRNA was performed to assess the flexibility of RNA complexing of the NUX019 using modifications to the conserved regions of NUX019 Short (pre-crRNA) on both the stem and the loop. The sequences of the pre-crRNAs tested are listed on Table 5. These short crRNAs were cloned into crRNA vectors as described in Example 1, and targeted guide vectors were created for each crRNA using the FANCF1 targeting sequence as described in Example 1.

TABLE 5
crRNA modifications tested with NUX019

| Label | pre-crRNA sequence |
|---|---|
| NUX019_−1 nt | GGCTAACAGCCTTTTAAAT |
| | TTCTACTGTTGTAGAT |
| | (SEQ ID NO: |
| | 316) |
| STEM_1_GC | GGCTAACAGCCTTTTAAA |
| | TTcCcACTGTGTGTgGgT |
| | (SEQ ID NO: |
| | 317) |
| STEM_Short | GGCTAACAGCCTTTTAAA |
| | TTTCTATGTGTTAGAT |
| | (SEQ ID NO: |
| | 318) |
| LOOP_Short | GGCTAACAGCCTTTTAAA |
| | TTTCTACTGTGTAGAT |
| | (SEQ ID NO: |
| | 319) |
| COMBO | GGCTAACAGCCTTTTAAA |
| | TTTCTATGTTAGAT |
| | (SEQ ID NO: 320) |
| STEM_Short_GC | GGCTAACAGCCTTTTAAA |
| | TTTCTcTGTGTgAGAT |
| | (SEQ ID NO: 321) |
| COMBO_GC | GGCTAACAGCCTTTTAA |
| | ATTTCTcTGTgAGAT |
| | (SEQ ID NO: 322) |

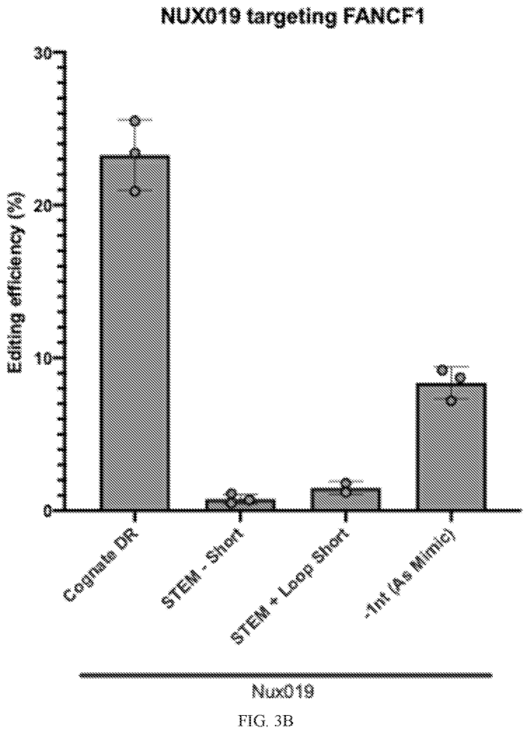

NUX019 was assayed for activity with the crRNA modifications in human cells as described in Example 3. Results are shown in FIGS. 3A-3B. The cognate direct repeat for NUX019 shows full editing activity while the −1 nt (As Mimic) condition shows partial editing, indicating some tolerance for loop size in the direct repeat. Truncating the direct repeat by removing one base pair from the stem or one base pair from the stem and one base pair from the loop was not tolerated and shows minimal editing by NUX019.

### Example 5

### Effect of Spacer Length on the Nuclease Activity

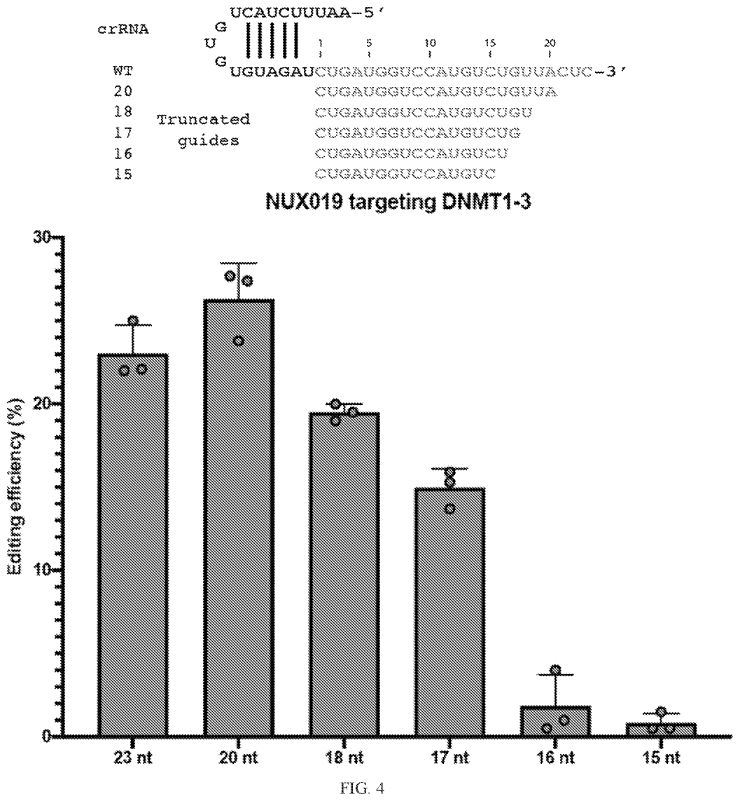

To assess the target spacer length requirements for the nucleases, targeting guide vectors were constructed with spacers targeting the DNMT1-3 region, spanning from 23 nt to 15 nt long. The list of oligonucleotides used to make these truncations are shown in Table 6. These oligonucleotides were ligated into the NUX019 Long crRNA vector and co-transfected with the NUX019 nuclease expression vector (Example 1) following the transfection and assay protocol of Example 3. Results of the nuclease editing efficiency are shown in FIG. 4. These results show that the editing efficiency was largely unaffected by a reduction in spacer length from 23 to 20 nucleotides. Editing efficiency modestly decreased as the spacer was reduced to 18 and 17 nucleotides but was significantly reduced when the spacer length was reduced to 16 or 15 nucleotides.

TABLE 6
Oligonucleotides used to make
spacer truncations

| Size | Target | Forward | Reverse |
|---|---|---|---|
| 23 | DNMT1-3 | AGATCTGATG | AGAAGAGTAA |
| | | GTCCATGTCT | CAGACATGGAC |
| | | GTTACTC | CATCAG |
| | | (SEQ ID | (SEQ ID |
| | | NO: 323) | NO: 324) |
| 20 | DNMT1-3 | AGATCTGATG | AGAATAACAG |
| | | GTCCATGTCT | ACATGGACCAT |
| | | GTTA | CAG |
| | | (SEQ ID | (SEQ ID |
| | | NO: 325) | NO: 326) |
| 18 | DNMT1-3 | AGATCTGATG | AGAAACAGAC |
| | | GTCCATGTCT | ATGGACCATCA |
| | | GT | G |
| | | (SEQ ID | (SEQ ID |
| | | NO: 327) | NO: 328) |
| 17 | DNMT1-3 | AGATCTGATG | AGAACAGACA |
| | | GTCCATGTCT | TGGACCATCAG |
| | | G (SEQ ID | (SEQ ID |
| | | NO: 329) | NO: 330) |
| 16 | DNMT1-3 | AGATCTGATG | AGAAAGACAT |
| | | GTCCATGTCT | GGACCATCAG |
| | | (SEQ ID | (SEQ ID |
| | | NO: 331) | NO: 332) |
| 15 | DNMT1-3 | AGATCTGAT | AGAAGACATG |
| | | GGTCCATGTC | GACCATCAG |
| | | (SEQ ID | (SEQ ID |
| | | NO: 333) | NO: 334) |

### Example 6

### Guide Multiplexing

To test the multiplexing abilities (e.g., multiple guides on a single RNA transcript) of the nucleases, a multiplexing guide vector was modified from the targeting guide vectors described in Example 1. The multiplexing guide vector contained four different targets (DNMT1, FANCF1, SMN2-CR3L and GRIN2B) separated by the nuclease specific short direct repeat (mature crRNA) to allow the nuclease to process the long RNA transcript into smaller guides and then complex with these guides and edit the target sites. The oligonucleotides used to make the multiplexing guide sequence for NUX019 is provided in Table 7.

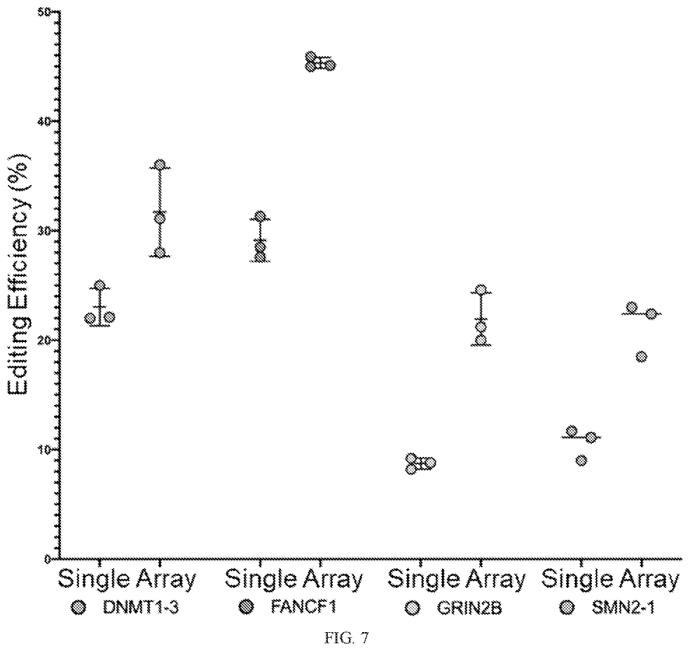

After amplification, the assembly of the multiplexing guide sequence was combined with the linearized vector by using the NEBuilder cloning kit, following NEB's protocol. The final vector was tested with the NUX019 nuclease expression vector (Example 1) using the methods in Example 3 with 150 ng of nuclease co-transfected with 50 ng of the multiplexing guide vector on a 96 well plate. Results of the multiplexing assay are shown in FIG. 7. For each target, editing is compared for single and multiplexed arrays. NUX019 is able to efficiently edit target sites using a multiplexed guide array. The multiplexed array produced a higher editing efficiency for all targets as compared with the single target.

TABLE 7
Oligonucleotides used to make multiplexing guide sequence

| Blocks | Primers |
|---|---|
| Block 1: | |
| Anneal Oligo 1F | gaaacaccgAATTTCTACTGTGTGTAGATCTGATGGTC |
| | CATGTCTGTTAC (SEQ ID NO: 335) |
| Anneal Oligo IR | CCCCGCCATCTACACACAGTAGAAATTGAGTAAC |
| | AGACATGGACCATCAG (SEQ ID NO: 336) |
| PCR amp oligo IF | TATATCTTGTGGAAAGGACgaaacacegAATTTCTACT |
| | G (SEQ ID NO: 337) |
| PCR amp oligo IR | AATTTAATCCCGGAACTGGACCCCGCCATCTACAC |
| | ACAGT (SEQ ID NO: 338) |
| Block 2: | |
| Anneal Oligo 2F | TCTACTGTGTGTAGATCAAAAGTAAGATTCACTTT |
| | CATAAATTTCTACTG (SEQ ID NO: 339) |
| Anneal Oligo 2R | TTATCTCCTTTCATTGAGCACATCTACACACAGTA |
| | GAAATTTATGAAAGT (SEQ ID NO: 340) |
| PCR amp oligo 2F | TCCAGTTCCGGGATTAAATTTCTACTGTGTGTAGA |
| | TCAAA (SEQ ID NO: 341) |
| PCR amp Oligo 2R | CTACACACAGTAGAAATTCCTTATCTCCTTTCATT |
| | GAGCA (SEQ ID NO: 342) |
| Block 3: | |
| Anneal Oligo 3F | ATTTTTTTgttttagagctagaaatagcaagttaaaat |
| | aaggctagtccg (SEQ ID NO: 343) |
| Anneal Oligo 3R | tgtctgcagaattggogcacgcgctAAAAacggacta |
| | gccttattttaac |
| | (SEQ ID NO: 344) |
| PCR amp oligo 3F | GGAATTTCTACTGTGTGTAGATTTTTTTgttttagag |
| | cta (SEQ ID NO: 345) |
| PCR amp Oligo 3R | Acggtacctctagagccatttgtctgcagaattggcg |
| | cac (SEQ ID NO: 346) |
| Vector linearization | |
| primers: | |
| VectorOpen Array 1R | GTCCTTTCCACaagatatataaagccaa |
| | (SEQ ID NO: 347) |
| VectorOpen Array 1F | Aatggctctagaggtaccgt |
| | (SEQ ID NO: 348) |

### Example 7

### Cleaving Site Characterization

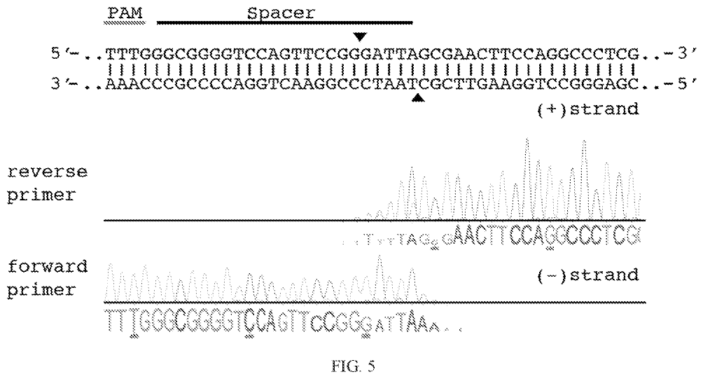

To determine how the nucleases cut double stranded DNA, a cleaving assay was performed using a protocol generally as set forth in Zetsche et al., (Zetsche B, et al., Cell. 2015 Oct. 22; 163(3):759-71, incorporated herein by reference in its entirety). A genomic DNA sequence bearing the FANCF1 target site with a TTTG PAM was amplified from wildtype HEK203T cells, cleaned up and quantified. HEK203T cells were co-transfected in a 12-well setup with NUX019 expression vector (670 ng) and NUX019 long Direct Repeat targeting FANCF1 guide vector (350 ng). Transfected cells were incubated for 48h and lysates were harvested using NP40 lysis buffer. 50 uL of lysis buffer was used for each well and 10 μL lysate aliquots were frozen at −80° C. For the cleavage assay, cleavage buffer was made to a 10× concentration and before commencing the cleavage reactions, a 1 mL aliquot of 10× cleavage buffer was supplemented with 10 μL of 1M DTT. The 1× cleavage buffer contained 10 mM HEPES, pH7.5, 150 mM NaCl, and 5 mM MgCl₂. 200 ng of amplified gDNA from WT cells was complexed with 10 μL of NUX019 cell lysate aliquot in a total of 20 μL reaction. Reactions were incubated at 37° C. for 1h and quenched by the addition of DNA loading dye with SDS and incubated at 70° C. for 10 minutes. The whole reaction was run on a 2% TAE agarose gel with SybrSafe. Gel bands for each piece of DNA were gel extracted and submitted to Sanger sequencing using forward and reverse primers. The Abl chromatogram was aligned to the sequence of the target genomic DNA to determine the end of the sequence on the plus and minus strand of DNA. Results are shown in FIG. 5. NUX019 cleaves the target strand between nucleotide position 23 at 24 and the non-target strand between nucleotide position 18 at 19 relative to the PAM domain, leaving a 5 nucleotide overhang on the 5′ ends of the double strand break.

### Example 8

### PAM Requirements

The sequence requirements for the PAM site for NUX019 and NUX003 can be assayed using the protocol of Walton et al. (Walton R T, et al., Science. 2020 Apr. 17; 368(6488): 290-296, incorporated herein by reference in its entirety) with spacer 3 (as described by Walton et al.). NUX019 and NUX003 are assayed using their long direct repeat guide vectors. Guide vectors targeting DNMT1-3 are used as a control.

### Example 9

Effect of crRNA-Target DNA Mismatch on Nuclease Editing

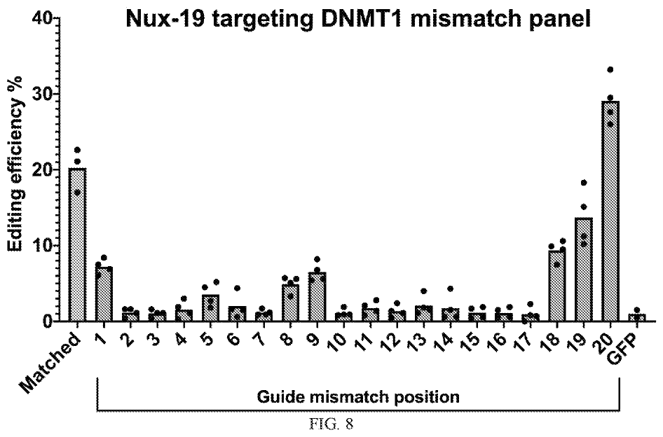

To assess the effect of sequence modifications in a target site, a series of targeting guide vectors was constructed with each with a single nucleotide mismatch across the 20 nucleotide DNMT1-3 target site. The mismatch series was constructed using the forward and reverse primers shown in Table 8. The annealed primers were ligated into the NUX019 Long crRNA vector (Example 1) and co-transfected with the NUX019 nuclease expression vector following the same protocol used in Example 3. Results are shown in FIG. 8. Generally, NUX019 was not tolerant of mutations in the guide at most positions. However, mismatch at the 3′ end (positions 18, 19 and 20) still permitted editing. The nuclease also exhibited some tolerances for mismatches at positions 1, 8 and 9, albeit with reduced editing efficiency.

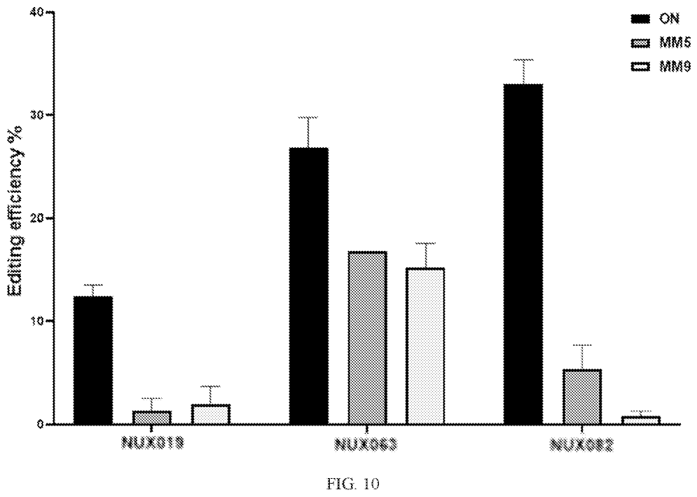

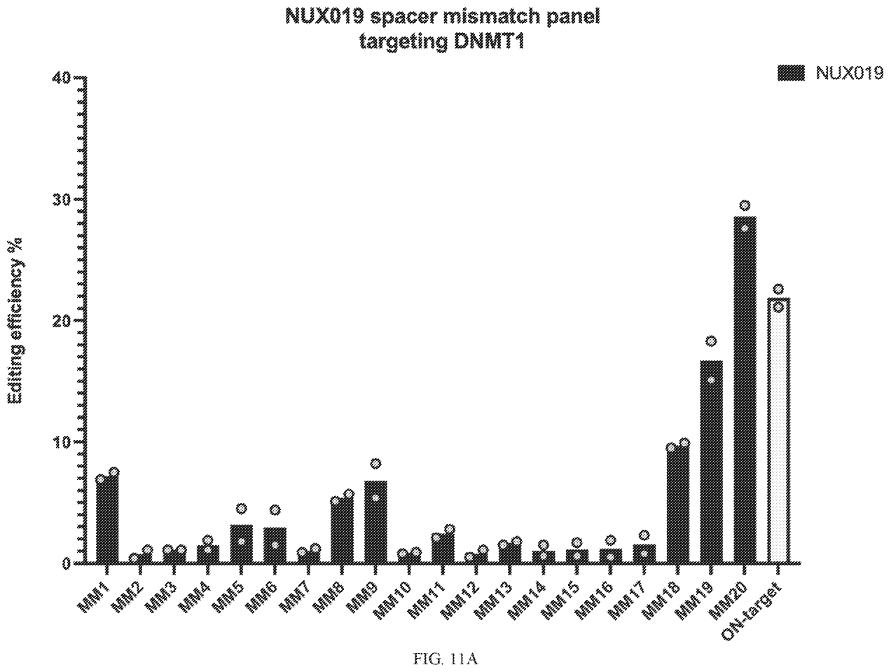

Following the methods used for NUX019 above, NUX063 and NUX082 were assessed for editing efficiency and on-target editing. Results are shown in FIG. 10 (editing efficiency) and FIG. 11 (on-target editing). NUX063 exhibited an increase in both on-target editing efficiency as well as an increase in editing with guides containing a mismatch at position 5 or 9 (MM5 or MM9, respectively). NUX082 further increased on-target editing while decreasing mismatch editing.

TABLE 8
Primers used to make mismatch targets
for NUX019 targeting guide vectors

| Target | Forward | Reverse |
|---|---|---|
| DNMT1-3_M1 | AGATGTGATGGTCCATGTCT | AGAATAACAGACATGGACCAT |
| | GTTA (SEQ ID NO: 349) | CAC (SEQ ID NO: 350) |
| DNMT1-3_M2 | AGATCAGATGGTCCATGTCT | AGAATAACAGACATGGACCAT |
| | GTTA (SEQ ID NO: 351) | CTG (SEQ ID NO: 352) |
| DNMT1-3_M3 | AGATCTCATGGTCCATGTCT | AGAATAACAGACATGGACCAT |
| | GTTA (SEQ ID NO: 353) | GAG (SEQ ID NO: 354) |
| DNMT1-3_M4 | AGATCTGTTGGTCCATGTCT | AGAATAACAGACATGGACCAA |
| | GTTA (SEQ ID NO: 355) | CAG (SEQ ID NO: 356) |
| DNMT1-3_MS | AGATCTGAAGGTCCATGTCT | AGAATAACAGACATGGACCTT |
| | GTTA (SEQ ID NO: 357) | CAG (SEQ ID NO: 358) |
| DNMT1-3_M6 | AGATCTGATCGTCCATGTCT | AGAATAACAGACATGGACGAT |
| | GTTA (SEQ ID NO: 359) | CAG (SEQ ID NO: 360) |
| DNMT1-3_M7 | AGATCTGATGCTCCATGTCT | AGAATAACAGACATGGAGCAT |
| | GTTA (SEQ ID NO: 361) | CAG (SEQ ID NO: 362) |
| DNMT1-3_M8 | AGATCTGATGGACCATGTCT | AGAATAACAGACATGGTCCAT |
| | GTTA (SEQ ID NO: 363) | CAG (SEQ ID NO: 364) |
| DNMT1-3_M9 | AGATCTGATGGTGCATGTCT | AGAATAACAGACATGCACCAT |
| | GTTA (SEQ ID NO: 365) | CAG (SEQ ID NO: 366) |
| DNMT1-3_M10 | AGATCTGATGGTCGATGTCT | AGAATAACAGACATCGACCAT |
| | GTTA (SEQ ID NO: 367) | CAG (SEQ ID NO: 368) |
| DNMT1-3_M11 | AGATCTGATGGTCCTTGTCT | AGAATAACAGACAAGGACCAT |
| | GTTA (SEQ ID NO: 369) | CAG (SEQ ID NO: 370) |
| DNMT1-3_M12 | AGATCTGATGGTCCAAGTCT | AGAATAACAGACTTGGACCAT |
| | GTTA (SEQ ID NO: 371) | CAG (SEQ ID NO: 372) |
| DNMT1-3_M13 | AGATCTGATGGTCCATCTCT | AGAATAACAGAGATGGACCAT |
| | GTTA (SEQ ID NO: 373) | CAG (SEQ ID NO: 374) |
| DNMT1-3_M14 | AGATCTGATGGTCCATGACT | AGAATAACAGTCATGGACCAT |
| | GTTA (SEQ ID NO: 375) | CAG (SEQ ID NO: 376) |
| DNMT1-3_M15 | AGATCTGATGGTCCATGTGT | AGAATAACACACATGGACCAT |
| | GTTA (SEQ ID NO: 377) | CAG (SEQ ID NO: 378) |
| DNMT1-3_M16 | AGATCTGATGGTCCATGTCA | AGAATAACTGACATGGACCAT |
| | GTTA (SEQ ID NO: 379) | CAG (SEQ ID NO: 380) |
| DNMT1-3_M17 | AGATCTGATGGTCCATGTCT | AGAATAAGAGACATGGACCAT |
| | CTTA (SEQ ID NO: 381) | CAG (SEQ ID NO: 382) |
| DNMT1-3_M18 | AGATCTGATGGTCCATGTCT | AGAATATCAGACATGGACCAT |
| | GATA (SEQ ID NO: 383) | CAG (SEQ ID NO: 384) |
| DNMT1-3_M19 | AGATCTGATGGTCCATGTCT | AGAATTACAGACATGGACCAT |
| | GTAA (SEQ ID NO: 385) | CAG (SEQ ID NO: 386) |
| DNMT1-3_M20 | AGATCTGATGGTCCATGTCT | AGAAAAACAGACATGGACCAT |
| | GTTT (SEQ ID NO: 387) | CAG (SEQ ID NO: 388) |

### Example 10

### NLS-TAG Positioning and Configuration

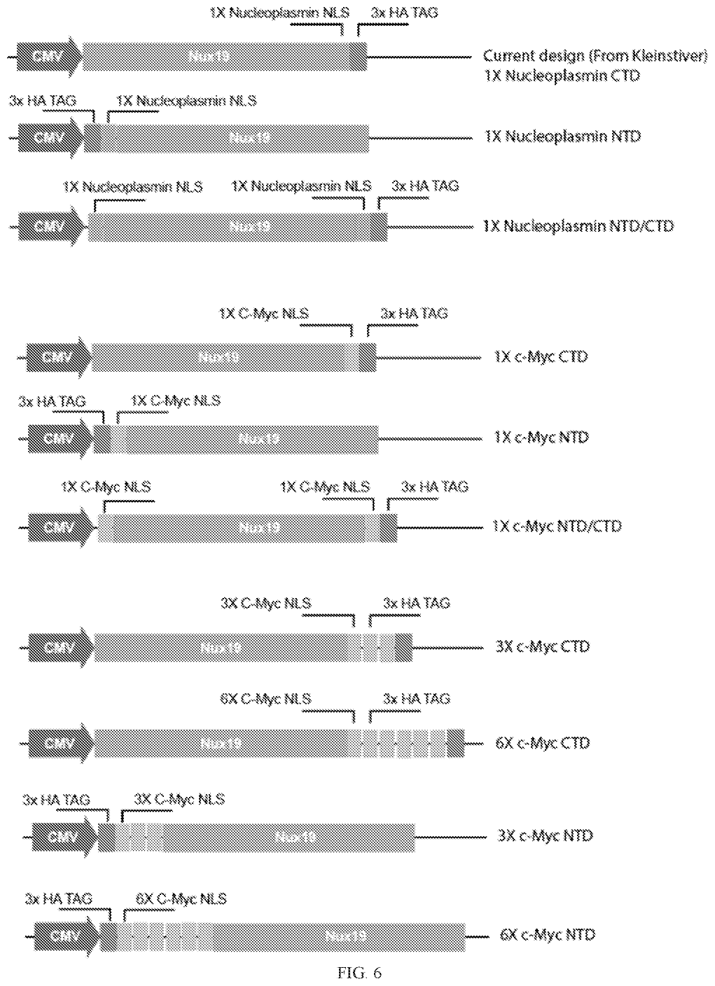

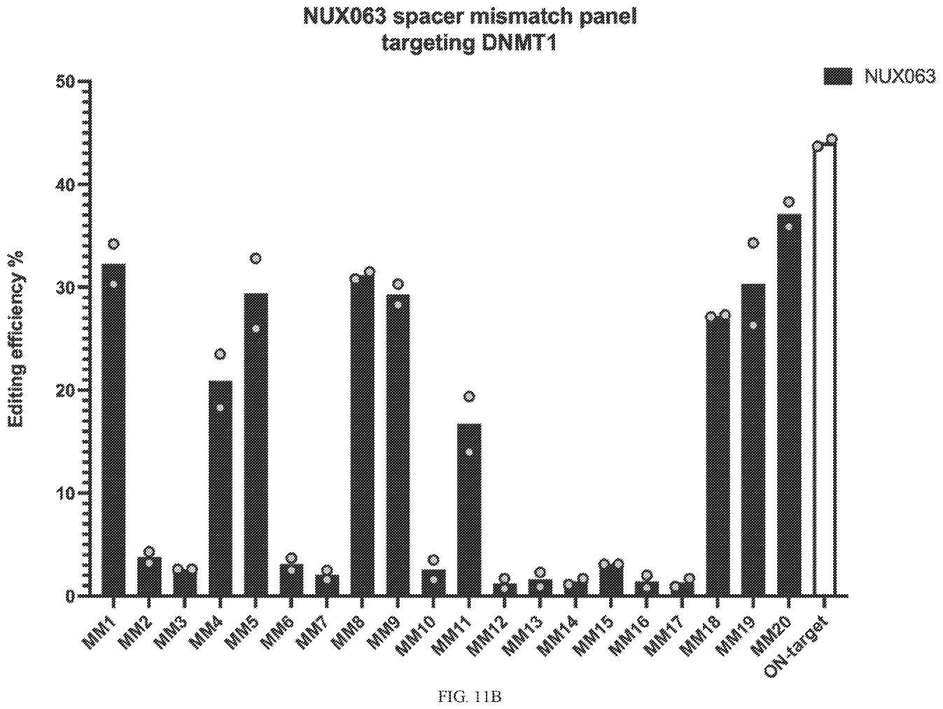

To test different nuclear localization signal (NLS) types and configurations, a series of expression vectors were constructed based on the CMV-Puro vector used for the nuclease expression vectors of Example 1. A schematic drawing of exemplary vectors for NUX019 is shown in FIG. 6. Sequences for components (gBlocks) for the vector series are provided in Table 9. These gBlocks were digested with NotI and BamHI restriction enzymes and used to replace the NUX019 cassette of the nuclease expression vector from Example 1. The constructs were assessed for activity following the same protocol used in Example 3. Results are shown in FIG. 12.

TABLE 9
NLS entry vector testing pieces made as gBlocks

| Testing | Sequence |
|---|---|
| 1x CTerm cMyc | CCAAGCTTGCCACCATGTACCTGGCCACTAGTGGCGGGAGCC |
| | CCGCCGCCAAGAGAGTGAAATTGGACGCCAGTGGGAGTGGA |
| | TACCCTTATGACGTGCCCGACTATGCCTACCCTTATGATGTCC |
| | CAGACTACGCTTATCCCTACGACGTACCGGATTATGCCGAATT |
| | CGGCAGTGGATGAGGATCCGCAGGCCTCTGC (SEQ ID NO: |
| | 389) |
| 3x Cterm cMyc | CCAAGCTTGCCACCATGTACCTGGCCACTAGTGGCGGGAGCC |
| | CCGCCGCCAAGAGAGTGAAATTGGACGGCGGCAGTCCCGCCG |
| | CTAAAAGGGTGAAGCTGGATGGTGGATCTCCCGCCGCCAAGA |
| | GAGTTAAGTTGGATGCCAGTGGGAGTGGATACCCTTATGACG |
| | TGCCCGACTATGCCTACCCTTATGATGTCCCAGACTACGCTTA |
| | TCCCTACGACGTACCGGATTATGCCGAATTCGGCAGTGGATG |
| | AGGATCCGCAGGCCTCTGC (SEQ ID NO: 390) |
| 6x Cterm cMyc | CCAAGCTTGCCACCATGTACCTGGCCACTAGTGGCGGGAGCC |
| | CCGCCGCCAAGAGAGTGAAATTGGACGGCGGCAGTCCCGCCG |
| | CTAAAAGGGTGAAGCTGGATGGTGGATCTCCCGCCGCCAAGA |
| | GAGTTAAGTTGGATGGCGGCTCCCCTGCAGCCAAACGGGTGA |
| | AACTCGACGGCGGGTCCCCGGCAGCCAAAAGGGTAAAGCTG |
| | GATGGTGGCTCTCCCGCAGCTAAACGGGTGAAGCTCGATGCC |
| | AGTGGGAGTGGATACCCTTATGACGTGCCCGACTATGCCTAC |
| | CCTTATGATGTCCCAGACTACGCTTATCCCTACGACGTACCGG |
| | ATTATGCCGAATTCGGCAGTGGATGAGGATCCGCAGGCCTCT |
| | GC (SEQ ID NO: 391) |
| 1x Nterm cMyc | CCCGCCAGCGGCCGCGCCACCATGGACTACCCTTATGACGTG |
| | CCCGACTATGCCTACCCTTATGATGTCCCAGACTACGCTTATC |
| | CCTACGACGTACCGGATTATGCCATGGGCGGGAGCCCCGCCG |
| | CCAAGAGAGTGAAATTGGACGGCGGCAGTAAGCTTGCCACCA |
| | TGTACCTGGCCACTAGTTACCTGGCCTAAGGATCCGCAGGCC |
| | TCTGCT (SEQ ID NO: 392) |
| 3x Nterm cMyc | CCCGCCAGCGGCCGCGCCACCATGGACTACCCTTATGACGTG |
| | CCCGACTATGCCTACCCTTATGATGTCCCAGACTACGCTTATC |
| | CCTACGACGTACCGGATTATGCCATGGGCGGGAGCCCCGCCG |
| | CCAAGAGAGTGAAATTGGACGGCGGCAGTCCCGCCGCTAAA |
| | AGGGTGAAGCTGGATGGTGGATCTCCCGCCGCCAAGAGAGTT |
| | AAGTTGGATGGCGGCTCCAAGCTTGCCACCATGTACCTGGCC |
| | ACTAGTTACCTGGCCTAAGGATCCGCAGGCCTCTGCT (SEQ ID |
| | NO: 393) |
| 6x Nterm cMyc | CCCGCCAGCGGCCGCGCCACCATGGACTACCCTTATGACGTG |
| | CCCGACTATGCCTACCCTTATGATGTCCCAGACTACGCTTATC |
| | CCTACGACGTACCGGATTATGCCATGGGCGGGAGCCCCGCCG |
| | CCAAGAGAGTGAAATTGGACGGCGGCAGTCCCGCCGCTAAA |
| | AGGGTGAAGCTGGATGGTGGATCTCCCGCCGCCAAGAGAGTT |
| | AAGTTGGATGGCGGCTCCCCTGCAGCCAAACGGGTGAAACTC |
| | GACGGCGGGTCCCCGGCAGCCAAAAGGGTAAAGCTGGATGG |
| | TGGCTCTCCCGCAGCTAAACGGGTGAAGCTCGATGCCAGTGG |
| | GAGTGGAAAGCTTGCCACCATGTACCTGGCCACTAGTTACCT |
| | GGCCTAAGGATCCGCAGGCCTCTGCT (SEQ ID NO: 394) |
| 1x Cterm cMyc + | CCCGCCAGCGGCCGCGCCACCatggacAAGCTTGCCACCATGTA |
| Nterm control | CCTGGCCACTAGTGGCGGGAGCCCCGCCGCCAAGAGAGTGAA |
| | ATTGGACGCCAGTGGGAGTGGATACCCTTATGACGTGCCCGA |
| | CTATGCCTACCCTTATGATGTCCCAGACTACGCTTATCCCTAC |
| | GACGTACCGGATTATGCCGAATTCGGCAGTGGATGAGGATCC |
| | GCAGGCCTCTGC (SEQ ID NO: 395) |
| 1x Nterm | CCCGCCAGCGGCCGCGCCACCATGGACTACCCTTATGACGTG |
| nucleoplasmin NLS | CCCGACTATGCCTACCCTTATGATGTCCCAGACTACGCTTATC |
| | CCTACGACGTACCGGATTATGCCATGGGCGGGAGCAAAAGGC |
| | CGGCGGCCACGAAAAAGGCCGGCCAGGCAAAAAAGAAAAAG |
| | GGCGGCAGTAAGCTTGCCACCATGTACCTGGCCACTAGTTAC |
| | CTGGCCTAAGGATCCGCAGGCCTCTGCT (SEQ ID NO: 396) |
| 1x Nterm 1x | CCCGCCAGCGGCCGCGCCACCATGGACATGGGGGGAGCCCC |
| Cterm cmyc | GCCGCCAAGAGAGTGAAATTGGACGGCGGCAGTAAGCTTGCC |
| | ACCATGTACCTGGCCACTAGTGGGGGAGCCCCGCCGCTAAA |
| | AGGGTGAAGCTGGATGCCAGTGGGAGTGGATACCCTTATGAC |
| | GTGCCCGACTATGCCTACCCTTATGATGTCCCAGACTACGCTT |
| | ATCCCTACGACGTACCGGATTATGCCGAATTCGGCAGTGGAT |
| | GAGGATCCGCAGGCCTCTGC (SEQ ID NO: 397) |
| 1x Nterm 1x | CCCGCCAGCGGCCGCGCCACCATGGACATGGGCGGGAGCAA |
| Cterm | AAGGCCGGCGGCCACGAAAAAGGCCGGCCAGGCAAAAAAGA |
| Nucleoplasmin | AAAAGGGCGGCAGTAAGCTTGCCACCATGTACCTGGCCACTA |
| | GTGGCGGGAGCAAAAGGCCGGCGGCCACGAAAAAGGCCGGC |
| | CAGGCAAAAAAGAAAAAGGCCAGTGGGAGTGGATACCCTTA |
| | TGACGTGCCCGACTATGCCTACCCTTATGATGTCCCAGACTAC |
| | GCTTATCCCTACGACGTACCGGATTATGCCGAATTCGGCAGT |
| | GGATGAGGATCCGCAGGCCTCTGC (SEQ ID NO: 398) |

### Example 11

### Comparison of NUX019 and AsCas12a for Direct Repeat Preferences

To explore the differences between NUX019 and NUX003 (AsCas12a), the two nucleases were compared on a series of direct repeat constructs following the protocols provided in Example 3.

A series of direct repeat vectors was constructed for the FANCF-1 using the short repeat sequences (Table 1) as described in Example 1.

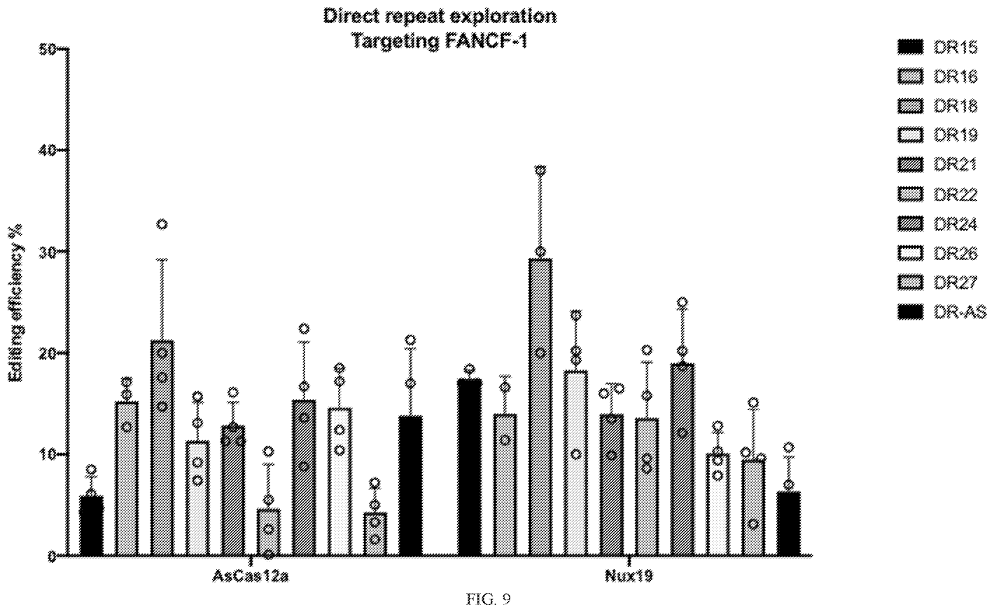

Results are shown in FIG. 9. Although both nucleases were active for editing using the full series of direct repeats, each nuclease had a distinct preference for some direct repeat sequences over others, and this preference differed as between NUX019 and AsCas12a.

### Example 12

mRNA Editing in Cell Culture

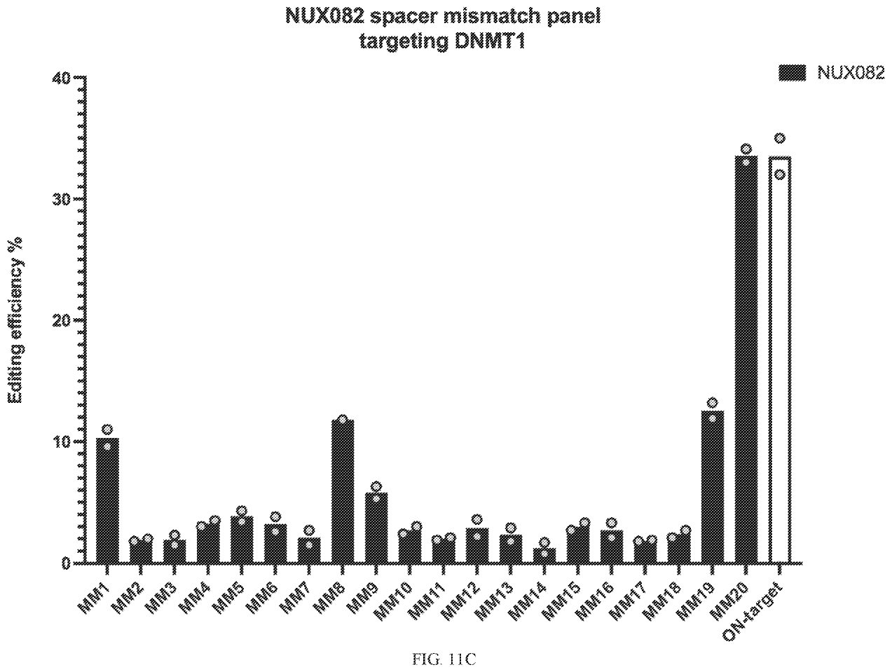

mRNA encoding NUX063 (SEQ ID NO: 405) along with an RNA guide targeting the Human DNMT1 gene (SEQ ID NO: 401) or a DNMT1 guide with a mismatch at position eight (SEQ ID NO: 402) were transfected into pre-seeded HEK293T cells using Mirus TransIT®-mRNA reagent (Mirus Bio). A total of 700 ng of RNA was transfected per 96-well of cells. Cells were allowed to incubate for 48 hours post transfection before harvesting and genomic DNA extraction using QuickExtract solution (Lucigen). About 200 ng of genomic DNA was amplified using KAPA HiFi polymerase and primers specific to the target region with Illumina adapters (Forward Primer-ACACTCTTTCCCTACACGACGCTCTTCCGATCTacgttcecttagcactetgcc (SEQ ID NO: 399), Reverse Primer-GACTGGAGTTCAGACGTGTGCTCTTCCGATCTGGGAGGGCAGAACTAGTCCT (SEQ ID NO: 400)). 2 μL of this first 25 μL PCR was input to a second PCR using Illumina P7 barcoded primers from New England BioLabs kit #E6609S. PCR products were checked on a 2% agarose gel for purity and cleaned via ZYMO kit #D4034. Samples were then sequenced on the Illumina MiSeq system, which returned 50-150k 150 bp paired-end reads per sample. Editing analysis was performed by CRISPResso2 (Clement, Kendell, et al. “CRISPResso2 provides accurate and rapid genome editing sequence analysis.” Nature biotechnology 37.3 (2019): 224-226). Editing efficiency was calculated as the percentage of nucleotide insertion or deletion mutations (indels) around the cut site without including substitution-only mutations. Results are shown in FIG. 13.

### Example 13

### LNP Editing in Cultured Primary Mouse Hepatocytes

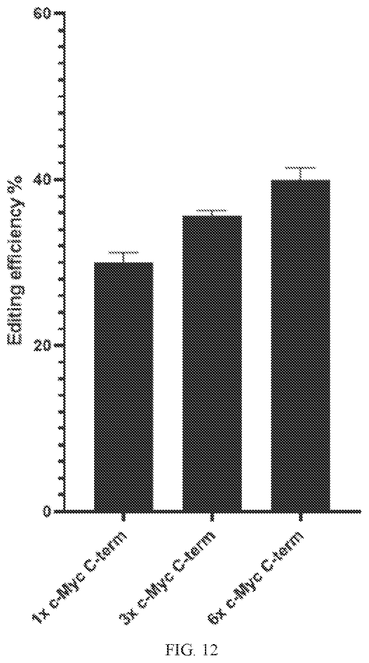

mRNA encoding NUX063 (SEQ ID NO: 405) along with an RNA guide targeting either the murine TTR gene (SEQ ID NO: 403) or PCSK9 gene (SEQ ID NO: 404) were encapsulated into a lipid nanoparticle (LNP) derived from GenVoy-ILM lipid mix (Precision Nanosystems). C57BL/6 mouse primary hepatocytes (Cell Biologics) were thawed and plated in 96 well tissue culture plates. The cells were incubated with LNPs at doses ranging from 2250 ng to 30 ng of RNA per well. Post LNP transfection, the cells were allowed to incubate for 24 hours before harvesting and genomic DNA extraction. Editing efficiencies were determined using Sanger sequencing and TIDE analysis as described in Example 3. Results are shown in FIG. 14.

### Example 14

### Editing in Mice Via Systemic Lipid Nanoparticle Delivery

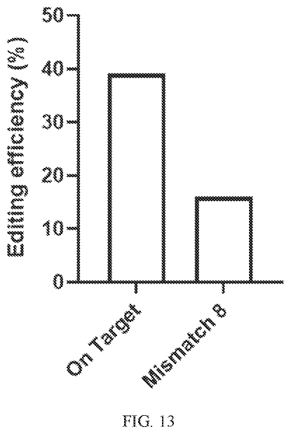

mRNA encoding NUX063 along with an RNA guide targeting the murine TTR gene were encapsulated into a lipid nanoparticle (LNP) derived from Gen Voy-ILM lipid mix (Precision Nanosystems). The LNP were buffer exchanged into phosphate buffer, concentrated, and sterile filtered. C57bl/6 mice were injected retro orbitally with LNP at 3 mg/kg dose. After seven days, the mouse livers were harvested, extracted for genomic DNA, and analyzed for TTR gene editing by next generation sequencing as described in Example 12. Results are shown in FIG. 15.

### Example 15

### Dose-Dependent RNP Editing of Human Cells

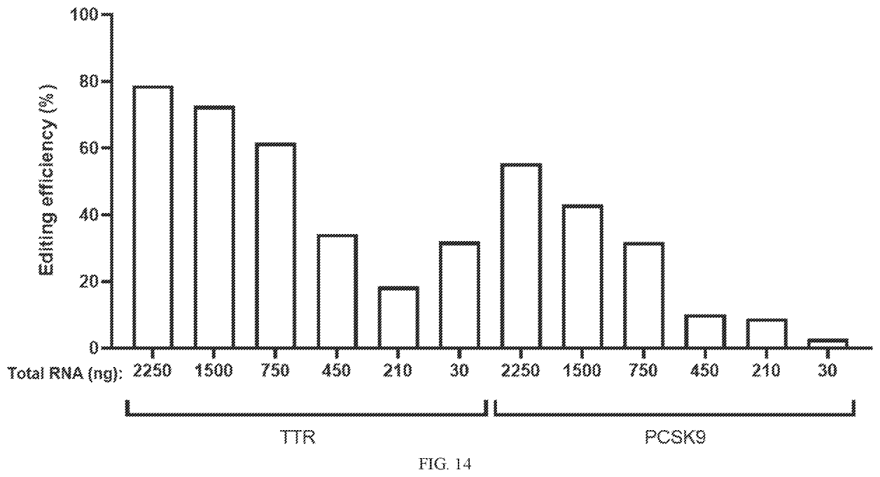

Various concentrations of NUX019 or NUX138 protein were complexed with guide RNA targeting DNMT1 for 20 minutes in phosphate buffered saline (PBS) at 25° C. The resulting ribonucleoprotein (RNP) complex was added to HEK293T cells and nucleofected using a Lonza 4D nucleofector system. Cells were allowed to incubate for 72 hours post-nucleofection before harvesting and genomic DNA extraction using Quick Extract solution. Editing efficiencies were determined using Sanger sequencing and TIDE analysis as described in Example 3. Results are shown in FIG. 16. NUX138 RNPs were approximately 2 orders of magnitude greater than NUX019 RNP in editing efficiency.

### Example 16

### Primary T Cell Editing and CAR T Generation

A CD-19 chimeric antigen receptor (CAR) integration cassette was constructed containing FMC63-CD28 hinge and transmembrane domain-CD28 intracellular domain and the CD3-zeta intracellular domain. This CAR sequence was flanked with over 600 bp on either end of homologous sequence surrounding the TCRA cut site and cloned in to an AAV6 compatible plasmid backbone. The AAV was packaged by transfecting the CAR transgene plasmid along with a helper (Aldevron pALD-X80) and capsid (Aldevron-pALD-AAV6) plasmid for AAV6 into AAVpro Hek 293T cells from Takara. Virus containing media was harvested 3 days post transfection and filtered before use in transduction. Human peripheral blood Pan-T cells (Stemcell Technologies) were thawed and expanded following manufacturer's instructions. After 48 hours, the cells were transduced with adeno-associated virus (AAV) containing a CD-19 chimeric antigen receptor (CAR) integration cassette. Cells were then nucleofected with a mixture of three ribonucleoproteins (RNPs) comprised of NUX138 and guide RNAs targeting the T cell receptor alpha chain (TCRA), beta-2-microglobulin (β2M), and Programmed Cell Death 1 (PD-1) genes as described in Example 15. After 72 hours, cells were harvested for analysis.

1×10⁶ cells for each nucleofection condition were harvested and stained with a cocktail containing antibodies against TCRA, β2M and PD-1 as well as CD25 to check for activation. All antibodies used are pre-conjugated with fluorophores and from BD biosciences: Hu CD3 FITC UCHT1 2STst, Hu CD8 APC HIT8A 25Tst, Hu CD25 APC BC96 25Tst, Hu CD279 (PD-1) PE-Cy7 EH12.1 100Tst and Hu Bta2-Microglobulin PE TU99 100Tst. For the staining procedure, cells were diluted to a concentration of 1-5×10⁶ cells/ml in ice cold FACS Buffer (PBS, 0.5-1% BSA) and stained in loBind 1.5 mL tubes. 100 μL of cell suspension was added to empty tubes on ice. 100 μl of Fc block added to each sample (Fc block diluted in FACS buffer at 1:50 ratio). Samples were incubated on ice for 20 min and centrifuged at 1500 rpm for 5 min at 4° C. Supernatant was discarded and the recommended manufacturer's concentration for the antibody cocktail was added. Samples were incubated in the dark at room temperature for 30 minutes. Cells were washed 3 times by centrifugation at 1500 rpm for 5 minutes and resuspended in 200 μl to 1 ml of ice cold FACS buffer. 100 μl 1-4% paraformaldehyde was added to each sample and tubes were incubated for 10-15 min at room temperature. After the incubation, samples were spun at 1500 rpm for 5 min, fixing agent was removed and cells were resuspended in 200 μl to 1 ml of ice cold PBS. Samples were analyzed on MACSquant analyzer 10.

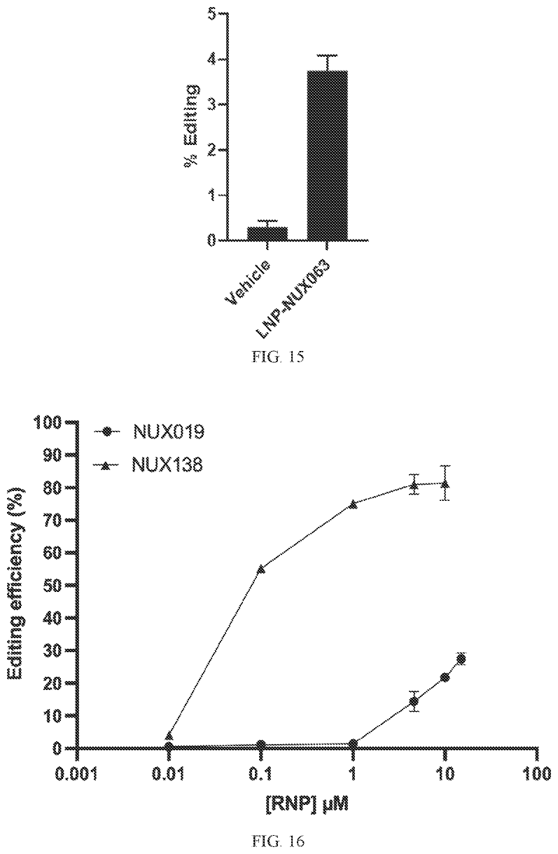

Genomic DNA was analyzed for editing by extracting edited CAR T cells using Quick Extract solution. Editing efficiencies were determined using Sanger sequencing and TIDE analysis as described in Example 3. Results are shown in FIG. 17A. Cell surface protein staining results are shown in FIG. 17B.

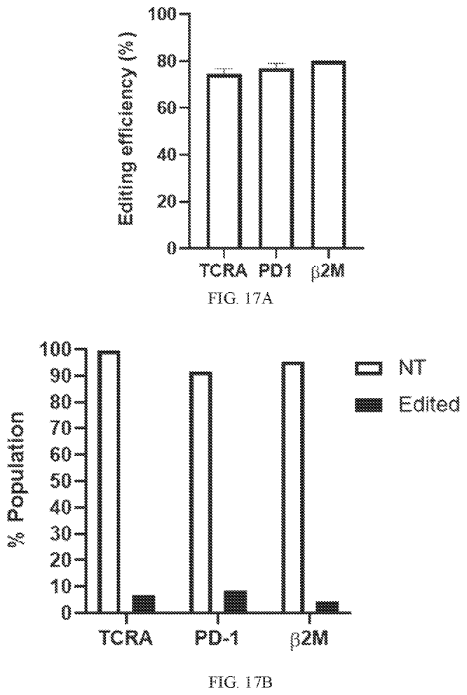

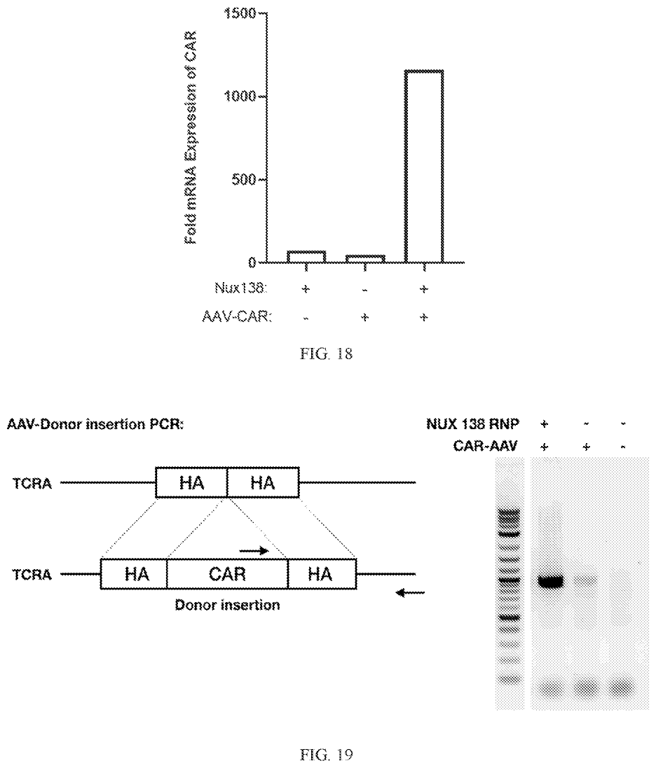

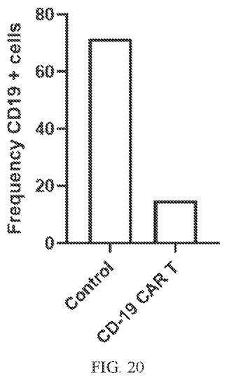

CD-19 CAR insertion was assessed by extraction of mRNA, generation of cDNA, and quantitative analysis using qPCR. Fold expression of the CD-19 CAR mRNA was calculated using the 2\hat{ }-ΔΔCT method. Results are shown in FIG. 18. Integration of the CD-19 CAR was assessed through genomic DNA amplification using PCR primers targeting the inserted CAR cassette. Results are shown in FIG. 19. The presence of a strong PCR product indicates integration of the CAR at the TCRA site. CD-19 CAR T activity was assessed using a NALM6 cancer cell model which expresses CD-19. NALM6 cells were incubated with engineered CD-19 CAR T cells for 48 hours. Viable NALM6 cells were assessed by staining for CD-19 using antibodies followed by flow cytometry analysis. Results are shown in FIG. 20.

### Example 17

### Enhancement of Nuclease Activity for Editing Efficiency and PAM Expansion

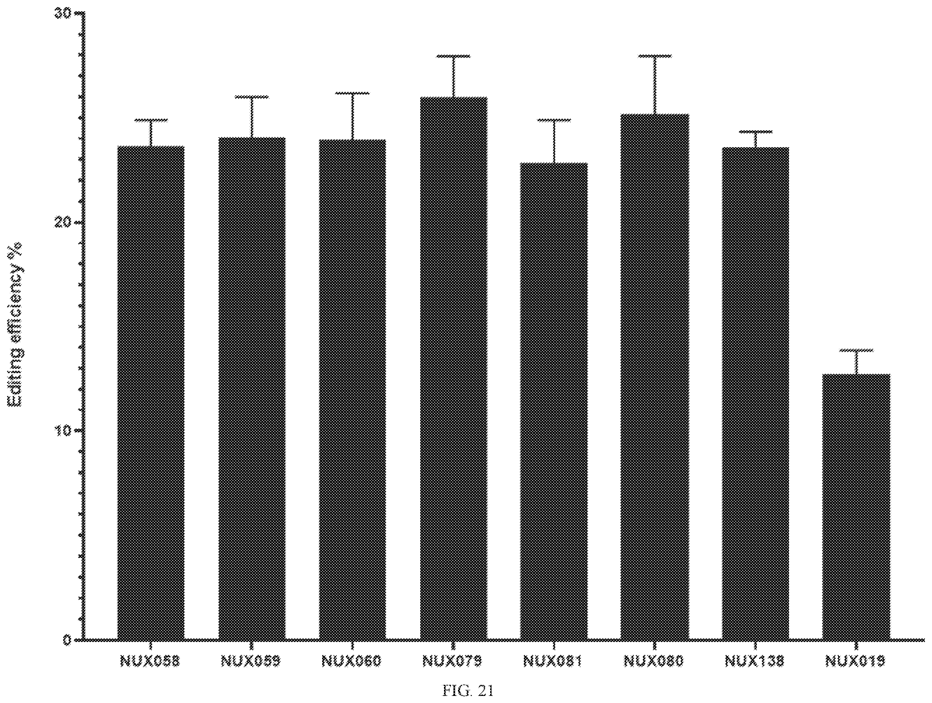

Amino acid substitutions at three distinct sites on NUX019 were assessed for editing activity and impact on PAM profile using the crRNA NUX019 Long (Table 1). Single substitutions NUX058, NUX059 and NUX069 and double substitutions NUX079, NUX081 and NUX063 were assessed for editing efficiency as described in Example 3. Results are shown in FIG. 21.

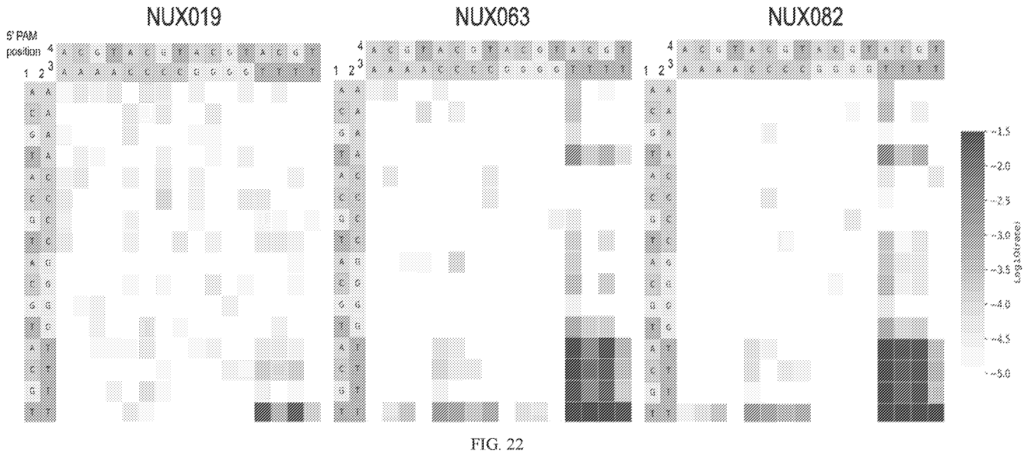

PAM sequence requirements for NUX019, NUX063, and NUX082 following the protocol and spacer 3 of Walton et al. (Walton R T, et al., Science. 2020 Apr. 17; 368(6488):290-296, incorporated herein by reference in its entirety). Results are shown in FIG. 22. The most preferred PAM sequences for each nuclease are listed in order of preference in Table 10.

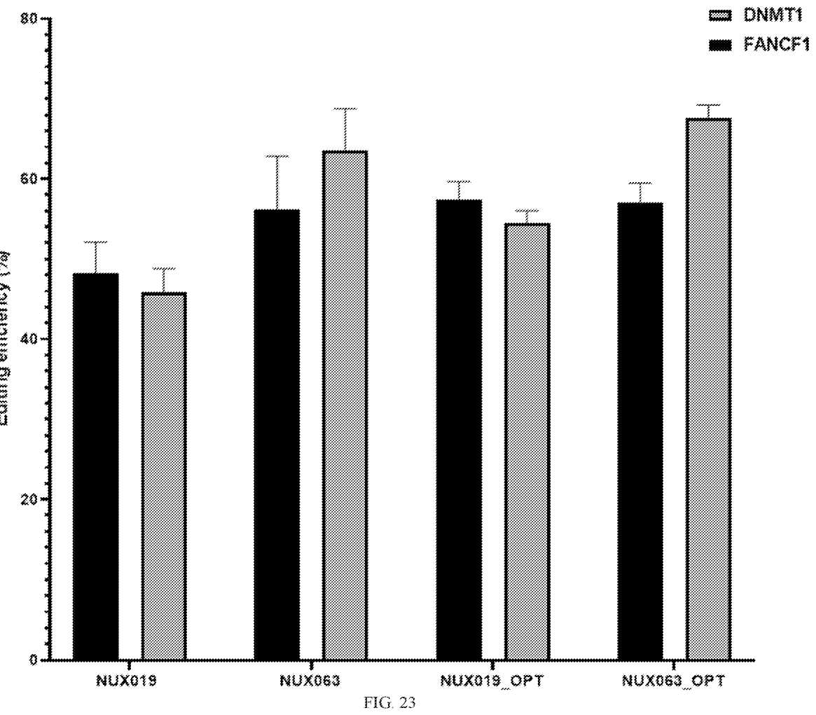

NUX019 and NUX063 were further optimized by including the OPT nuclear localization signal (NLS) and assessed for editing of the DNMT1 or FANCF1 site in HEK293T cells as described in Example 3. Results are shown in FIG. 23.

TABLE 10

| | Preferred |
|---|---|
| NUX | PAM |
| NUX019 | TTTA |
| NUX019 | TTTG |
| NUX063 | TTTA |
| NUX063 | TTTG |
| NUX063 | TTTC |
| NUX063 | CTTA |
| NUX063 | ATTA |
| NUX063 | GTTA |
| NUX063 | ATTG |
| NUX063 | TTTT |
| NUX063 | CTTG |
| NUX063 | GTTG |
| NUX082 | CTTA |
| NUX082 | TTTG |
| NUX082 | TTTA |
| NUX082 | TTTC |
| NUX082 | ATTA |
| NUX082 | GTTA |
| NUX082 | TTTT |
| NUX082 | CTTG |
| NUX082 | GTTG |
| NUX082 | ATTG |
| NUX082 | ATTC |
| NUX082 | CTTC |
| NUX082 | GTTC |

### Example 18

### Comparison of Editing Efficiency

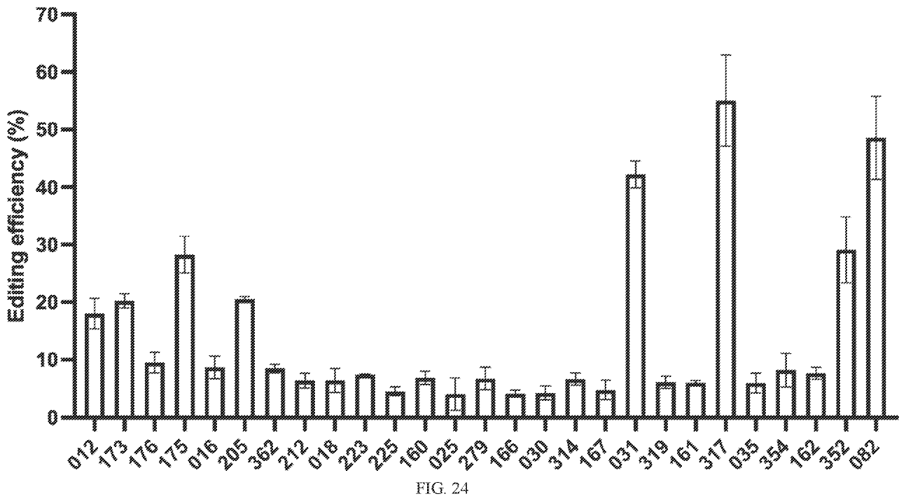

Following the methods of Example 3, nucleases were tested for editing efficiency by transient transfection into in HEK293 cells using the DMNT1 target. Results are shown in FIG. 24.

Sequences

| NUX | SEQ ID NO: |
|---|---|
| NUX012 | 1 |
| NUX013 | 2 |
| NUX014 | 3 |
| NUX015 | 4 |
| NUX016 | 5 |
| NUX017 | 6 |
| NUX018 | 7 |
| NUX019 | 8 |
| NUX020 | 9 |
| NUX021 | 10 |
| NUX022 | 11 |
| NUX023 | 12 |
| NUX024 | 13 |
| NUX025 | 14 |
| NUX026 | 15 |
| NUX027 | 16 |
| NUX028 | 17 |
| NUX030 | 18 |
| NUX031 | 19 |
| NUX032 | 20 |
| NUX033 | 21 |
| NUX034 | 22 |
| NUX035 | 23 |
| NUX003 | 24 |
| (AsCas12a) | |
| NUX058 | 26 |
| NUX059 | 27 |
| NUX060 | 28 |
| NUX061 | 29 |
| NUX063 | 32 |
| NUX082 | 33 |
| NUX138 | 34 |
| NUX081 | 35 |
| NUX079 | 36 |
| NUX169 | 37 |
| NUX170 | 38 |
| NUX171 | 39 |
| NUX172 | 40 |
| NUX173 | 41 |
| NUX174 | 42 |
| NUX175 | 43 |
| NUX176 | 44 |
| NUX177 | 45 |
| NUX178 | 46 |
| NUX179 | 47 |
| NUX180 | 48 |
| NUX181 | 49 |
| NUX182 | 50 |
| NUX183 | 51 |
| NUX184 | 52 |
| NUX185 | 53 |
| NUX186 | 54 |
| NUX187 | 55 |
| NUX188 | 56 |
| NUX189 | 57 |
| NUX190 | 58 |
| NUX191 | 59 |
| NUX192 | 60 |
| NUX193 | 61 |
| NUX194 | 62 |
| NUX195 | 63 |
| NUX196 | 64 |
| NUX197 | 65 |
| NUX198 | 66 |
| NUX199 | 67 |
| NUX200 | 68 |
| NUX201 | 69 |
| NUX202 | 70 |
| NUX203 | 71 |
| NUX204 | 72 |
| NUX205 | 73 |
| NUX206 | 74 |
| NUX207 | 75 |
| NUX208 | 76 |
| NUX209 | 77 |
| NUX210 | 78 |
| NUX211 | 79 |
| NUX212 | 80 |
| NUX213 | 81 |
| NUX214 | 82 |
| NUX215 | 83 |
| NUX216 | 84 |
| NUX217 | 85 |
| NUX218 | 86 |
| NUX219 | 87 |
| NUX220 | 88 |
| NUX221 | 89 |
| NUX222 | 90 |
| NUX223 | 91 |
| NUX224 | 92 |
| NUX225 | 93 |
| NUX226 | 94 |
| NUX227 | 95 |
| NUX228 | 96 |
| NUX229 | 97 |
| NUX160 | 98 |
| NUX230 | 99 |
| NUX231 | 100 |
| NUX232 | 101 |
| NUX233 | 102 |
| NUX232 | 103 |
| NUX234 | 104 |
| NUX235 | 105 |
| NUX236 | 106 |
| NUX237 | 107 |
| NUX238 | 108 |
| NUX239 | 109 |
| NUX240 | 110 |
| NUX241 | 111 |
| NUX242 | 112 |
| NUX243 | 113 |
| NUX244 | 114 |
| NUX245 | 115 |
| NUX246 | 116 |
| NUX247 | 117 |
| NUX248 | 118 |
| NUX249 | 119 |
| NUX250 | 120 |
| NUX251 | 121 |
| NUX252 | 122 |
| NUX253 | 123 |
| NUX254 | 124 |
| NUX255 | 125 |
| NUX256 | 126 |
| NUX257 | 127 |
| NUX258 | 128 |
| NUX259 | 129 |
| NUX260 | 130 |
| NUX261 | 131 |
| NUX262 | 132 |
| NUX263 | 133 |
| NUX264 | 134 |
| NUX265 | 135 |
| NUX266 | 136 |
| NUX267 | 137 |
| NUX268 | 138 |
| NUX269 | 139 |
| NUX270 | 140 |
| NUX271 | 141 |
| NUX272 | 142 |
| NUX273 | 143 |
| NUX274 | 144 |
| NUX275 | 145 |
| NUX276 | 146 |
| NUX277 | 147 |
| NUX278 | 148 |
| NUX279 | 149 |
| NUX280 | 150 |
| NUX281 | 151 |
| NUX166 | 152 |
| NUX282 | 153 |
| NUX283 | 154 |
| NUX284 | 155 |
| NUX285 | 156 |
| NUX286 | 157 |
| NUX287 | 158 |
| NUX288 | 159 |
| NUX289 | 160 |
| NUX290 | 161 |
| NUX291 | 162 |
| NUX292 | 163 |
| NUX293 | 164 |
| NUX294 | 165 |
| NUX295 | 166 |
| NUX296 | 167 |
| NUX297 | 168 |
| NUX298 | 169 |
| NUX299 | 170 |
| NUX300 | 171 |
| NUX301 | 172 |
| NUX302 | 173 |
| NUX303 | 174 |
| NUX304 | 175 |
| NUX305 | 176 |
| NUX306 | 177 |
| NUX307 | 178 |
| NUX308 | 179 |
| NUX309 | 180 |
| NUX310 | 181 |
| NUX311 | 182 |
| NUX312 | 183 |
| NUX313 | 184 |
| NUX314 | 185 |
| NUX315 | 186 |
| NUX316 | 187 |
| NUX167 | 188 |
| NUX361 | 189 |
| NUX317 | 190 |
| NUX318 | 191 |
| NUX319 | 192 |
| NUX320 | 193 |
| NUX321 | 194 |
| NUX322 | 195 |
| NUX323 | 196 |
| NUX161 | 197 |
| NUX324 | 198 |
| NUX325 | 199 |
| NUX326 | 200 |
| NUX327 | 201 |
| NUX328 | 202 |
| NUX329 | 203 |
| NUX330 | 204 |
| NUX331 | 205 |
| NUX332 | 206 |
| NUX333 | 207 |
| NUX334 | 208 |
| NUX335 | 209 |
| NUX336 | 210 |
| NUX337 | 211 |
| NUX338 | 212 |
| NUX339 | 213 |
| NUX340 | 214 |
| NUX341 | 215 |
| NUX342 | 216 |
| NUX343 | 217 |
| NUX344 | 218 |
| NUX345 | 219 |
| NUX346 | 220 |
| NUX347 | 221 |
| NUX348 | 222 |
| NUX349 | 223 |
| NUX350 | 224 |
| NUX351 | 225 |
| NUX352 | 226 |
| NUX353 | 227 |
| NUX354 | 228 |
| NUX355 | 229 |
| NUX356 | 230 |
| NUX357 | 231 |
| NUX358 | 232 |
| NUX162 | 233 |
| NUX359 | 234 |
| NUX362 | 235 |
| NUX360 | 236 |

AsCas12a short direct repeat (DR-As)
(SEQ ID NO: 25)
AATTTCTACTCTTGTAGATAT
DNMT1 guide
(SEQ ID NO: 401)
AAUUUCUACUGUGUGUAGACUGAUGGUCCAUGUCUGUUA
DNMT1 mismatch 8
(SEQ ID NO: 402)
AAUUUCUACUGUGUGUAGACUGAUGGaCCAUGUCUGUUA
PCSK9 guide
(SEQ ID NO: 403)
AAUUUCUACUGUGUGUAGAUUCAAUCUGUAGCCUCUGGGUCU
TTR guide
(SEQ ID NO: 404)
AAUUUCUACUGUGUGUAGAUCCUCGCUGGACUGGUAUUUGU
OPT NLS
(SEQ ID NO: 417)
GRSSDDEATADSQHAAPPKKKRKVGGSGGSGGSGGSGGSGGS
GGSGGSLEGSYPYDVPDYAYPYDVPDYAYPYDVPDYA

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

What is claimed is:

1. A engineered nuclease comprising an amino acid sequence having at least 70% identity, at least 80% identity, at least 90% identity, at least 95% identity or at least 99% identity to any of SEQ ID NOs: 1-23 or 32-35.

2. The engineered nuclease of claim 1, wherein the amino acid sequence has at least 90% identity to any of SEQ ID NOs: 1-23 or 32-35.

3. The engineered nuclease of claim 1 or 2, wherein the amino acid sequence has less than 50% sequence identity with SEQ ID NO: 24.

4. The engineered nuclease of any of claims 1-3, wherein the amino acid sequence is selected from the group consisting of SEQ ID NOs: 1-23, 26-29, and 32-236.

5. The engineered nuclease of any of claims 1-4, further comprising a nuclear localization sequence (NLS).

6. A nucleic acid molecule comprising a sequence encoding the engineered nuclease of any of claims 1-5.

7. A composition comprising a nuclease comprising an amino acid sequence having at least 70% identity to any of SEQ ID NOs: 1-23 or 32-35 or a nucleic acid molecule comprising a sequence encoding the nuclease.

8. The composition of claim 7, wherein the nuclease comprises an amino acid sequence having at least 90% identity to any of SEQ ID NOs: 1-23 or 32-35.

9. The composition of claim 7 or 8, wherein the amino acid sequence has less than 50% sequence identity with SEQ ID NO: 24.

10. The composition of any of claims 7-9, wherein the nuclease comprises an amino acid sequence selected from the group consisting of SEQ ID NOs: 1-23, 26-29, and 32-236.

11. The composition of any of claims 7-10, wherein the nucleic acid molecule comprising a sequence encoding the nuclease comprises a messenger RNA or a vector.

12. The composition of any of claims 7-11, wherein the nuclease further comprises a nuclear localization sequence (NLS).

13. The composition of any of claims 7-12, wherein the composition further comprises at least one guide RNA (gRNA) or a nucleic acid comprising a sequence encoding the gRNA.

14. The composition of claim 13, wherein the at least one gRNA comprises a non-naturally occurring gRNA.

15. The composition of claim 13 or 14, wherein the at least one gRNA is encoded in a CRISPR RNA array.

16. A system for modifying a target nucleic acid comprising:
a) a nuclease comprising an amino acid sequence having at least 70% identity, at least 80% identity, at least 90% identity, at least 95% identity or at least 99% identity to any of SEQ ID NOs: 1-23, 26-29, or 32-236 or a nucleic acid molecule comprising a sequence encoding the nuclease; and
b) at least one guide RNA (gRNA) complementary to at least a portion of the target nucleic acid or a nucleic acid comprising a sequence encoding the at least one gRNA.

17. The system of claim 16, wherein the nucleic acid molecule encoding each one or both of the nuclease and the at least one gRNA comprises a messenger RNA, a vector, or a combination thereof.

18. The system of claim 16 or 17, wherein the nuclease and the at least one gRNA are encoded on the same nucleic acid.

19. The system of any of claims 16-18, wherein the amino acid sequence has less than 50% sequence identity with SEQ ID NO: 24.

20. The system of any of claims 16-19, wherein the nuclease further comprises a nuclear localization sequence (NLS).

21. The system of any of claims 16-20, wherein the at least one gRNA comprises a non-naturally occurring gRNA.

22. The system of any of claims 16-21, wherein the at least one gRNA is encoded in a CRISPR RNA array.

23. The system of any of claims 16-22, wherein the system further comprises a target nucleic acid.

24. The system of any of claims 16-23, wherein the system is a cell-free system.

25. A cell comprising the system of any of claims 16-23.

26. The cell of claim 25, wherein the cell is a prokaryotic or eukaryotic cell.

27. A method of modifying a target nucleic acid sequence comprising contacting the target nucleic acid with an engineered nuclease of claims 1-5, a composition of any of claims 7-15, or a system of any of claims 16-23.

28. The method of claim 27, wherein the target nucleic acid sequence is in a cell.

29. The method of claim 28, wherein the cell is a prokaryotic or eukaryotic cell.

30. The method of claim 28, wherein the cell is a human cell.

31. The method of any of claims 27-30, wherein contacting a target nucleic acid sequence comprises introducing the system into the cell.

32. The method of claim 31, wherein introducing the system into the cell comprises administering the system to a subject.

33. The method of any of claims 27-32, wherein the target nucleic acid sequence encodes a gene product.

34. A method of generating a cell that expresses a recombinant receptor comprising introducing into the cell:
a nuclease comprising an amino acid sequence having at least 70% identity, at least 80% identity, at least 90% identity, at least 95% identity or at least 99% identity to any of SEQ ID NOs: 1-23, 26-29, or 32-236 or a nucleic acid molecule comprising a sequence encoding the nuclease;
at least one guide RNA (gRNA) complementary to at least a portion of a target nucleic acid or a nucleic acid comprising a sequence encoding the at least one gRNA; and
a nucleic acid encoding the recombinant receptor.

35. The method of claim 34, wherein the recombinant receptor is a T cell receptor (TCR) or a chimeric antigen receptor (CAR).

36. The method of claim 34 or 35, wherein the system and the recombinant receptor are encoded by separate nucleic acids.

37. The method of any of claims 34-36, wherein the cell is a T cell.

38. The method of claim 37, wherein the T cell is from a subject.

39. The method of claim 37 or 38, wherein the T cell is expanded in vitro.

40. The method of any of claims 34-39, wherein the nucleic acid encoding the recombinant receptor is integrated into genomic DNA of the cell.

41. A T cell comprising:
a system of any of claims 16-23;
and a recombinant receptor or a nucleic acid encoding the recombinant receptor.

42. The T cell of claim 41, wherein the T cell is from a subject.

43. A method modifying a target nucleic acid in a plant comprising providing to the plant, or a plant cell, seed, fruit, plant part, or propagation material of the plant:
a nuclease comprising an amino acid sequence having at least 70% identity, at least 80% identity, at least 90% identity, at least 95% identity or at least 99% identity to any of SEQ ID NOs: 1-23, 26-29, or 32-236 or a nucleic acid molecule comprising a sequence encoding the nuclease; and
at least one guide RNA (gRNA) complementary to at least a portion of the target nucleic acid or a nucleic acid comprising a sequence encoding the at least one gRNA.

44. The method of claim 43, wherein the nucleic acid molecule encoding each one or both of the nuclease and the at least one gRNA comprises a messenger RNA, a vector, or a combination thereof.

45. The method of claim 43 or 44, wherein the nuclease and the at least one gRNA are encoded on a single nucleic acid.

46. The method of any of claims 43-45, wherein the amino acid sequence has less than 50% sequence identity with SEQ ID NO: 24.

47. The method of any of claims 43-46, wherein the nuclease further comprises a nuclear localization sequence (NLS).

48. The method of any of claims 43-47, wherein the at least one gRNA comprises a non-naturally occurring gRNA.

49. The method of any of claims 43-48, wherein the at least one gRNA is encoded in a CRISPR RNA array.

50. The method of any of claims 43-49, further comprising providing to the plant a donor polynucleotide.

51. The method of any of claims 43-50, wherein the nucleic acid encodes a gene product.

52. The method of any of claims 43-51, wherein the plant is a monocot or a dicot.

53. The method of any of claims 43-52, wherein the plant is a grain crop, a fruit crop, a forage crop, a root vegetable crop, a leafy vegetable crop, a flowering plant, a conifer, an oil crop, a plant used in phytoremediation, an industrial crop, a medicinal crop, or a laboratory model plant.

54. The method of any of claims 43-53, wherein the nucleic acid molecule comprising a sequence encoding the nuclease and the at least one gRNA or the nucleic acid encoding the at least one guide RNA are provided via Agrobacterium-mediated transformation.

55. The method of any of claims 43-54, wherein the method confers one or more of the following traits to the plant or a plant cell, seed, fruit, plant part, or propagation material of the plant: herbicide tolerance, drought tolerance, male sterility, insect resistance, abiotic stress tolerance, modified fatty acid metabolism, modified carbohydrate metabolism, modified seed yield, modified oil percent, modified protein content, disease resistance, cold and frost tolerance, improved taste, increased germination, increased micronutrient uptake, improved flower longevity, modified fragrance, modified nutritional value, modified fruit or flower size or number, modified growth, and modified plant size.

56. A method for treating a disease or disorder in a subject comprising administering to the subject in need thereof:
a cell comprising a recombinant receptor or a nucleic acid encoding the recombinant receptor, the nuclease, or a nucleic acid encoding thereof, and at least one gRNA, or a nucleic acid encoding thereof; or
a nuclease comprising an amino acid sequence having at least 70% identity, at least 80% identity, at least 90% identity, at least 95% identity or at least 99% identity to any of SEQ ID NOs: 1-23, 26-29, or 32-236 or a nucleic acid molecule comprising a sequence encoding the nuclease; and
at least one guide RNA (gRNA) complementary to at least a portion of a target nucleic acid or a nucleic acid comprising a sequence encoding the at least one gRNA.

57. The method of claim 56, wherein the nucleic acid molecule encoding each one or both of the nuclease and the at least one gRNA comprises a messenger RNA, a vector, or a combination thereof.

58. The method of claim 56 or 57, wherein the nuclease and the at least one gRNA are encoded on a single nucleic acid.

59. The method of any of 56-58, wherein the amino acid sequence has less than 50% sequence identity with SEQ ID NO: 24.

60. The method of any of claims 56-59, wherein the nuclease further comprises a nuclear localization sequence (NLS).

61. The method of any of claims 56-60, wherein the at least one gRNA comprises a non-naturally occurring gRNA.

62. The method of any of claims 56-61, wherein the at least one gRNA is encoded in a CRISPR RNA array.

63. The method of any of claims 56-62, wherein the cell is a T cell.

64. The method of claim 63, wherein the T cell is from a subject.

65. The method of claim 63 or 64, wherein the T cell is expanded in vitro.

66. The method of any of claims 56-65, wherein the nucleic acid encoding the recombinant receptor is integrated into genomic DNA of the cell.

67. The method of any of claims 56-66, wherein the target nucleic acid is a disease-associated gene.

68. The method of any of claims 56-67, further comprising administering a donor polynucleotide.

69. The method of claim 68, wherein the donor polynucleotide comprises a therapeutic protein, functional gene product, or a combination thereof.

70. The method of any of claims 56-69, wherein the subject is a human.

71. The method of any of claims 56-70, further comprising administering a therapeutic agent.

72. Use of an engineered nuclease of claims 1-5, a composition of any of claims 7-15, or a system of any of claims 16-24 for modifying a target nucleic acid sequence.

73. Use of an engineered nuclease of claims 1-5, a composition of any of claims 7-15, or a system of any of claims 16-24 for modifying a target nucleic acid in a plant.

74. Use of an engineered nuclease of claims 1-5, a composition of any of claims 7-15, a system of any of claims 16-24, or a T cell of claims 41-42 for treating a disease or disorder in a subject.


SEQUENCE LISTING (488 sequences)

SEQ ID NO: 1 - protein, 1197 aa, unidentified
MNIYENFTNMYQVNKTIRMGLKPICKTDENIAKFLEEDKERSEKYKIAKKIIDKENRAFIEDRLKDFSISGLDEYLELLKQKKDITKIQKKMRDEISKQLKGFPQFDSKYKFQYITDKEDTEILEYFKKFTTFFTGFNSNRENVYSKEDISTSIGHRIIHENLPKFISNFRILNKAIEALGTGKINEDFKNNEINVTVEELNKIDYFNKVLTQSGIDLYNNLIGILNQNINLYNQQQKVKKNKIGKLETLHKQILSEKDKVSFIEEFAEDNQLLKCIDEYFKEKSCLINVDLKNLLENIDTYSLNGIFIKNDKSLKNISIYLYKDWGYISNLINEEYDYKHKNKVKDDKYYEKRKKAIDKIKYFSIGYIDELLKDKNVPMVECYFKEKINLVVKEFIDYLNKFNEYKFTNELKTDEIAVEIIKNLCDSIKDVQEIVKPLIVIENDKDDSFYGEINYIWDELNKFNKIYNMVRNYLTKKDYIDEKIRLTFNNPQLLGGWSYKKEADYSSFILKDNKYYYLAIMNPNSKSKLKDLKKPENDGDTIYKMKYFQASDPAKSIPNLMVINGKTEKKNGRKDNDGENRELERLRNEYLPTEINEIRKNKAYLRSSSNFSNEALVKYIDYYKERVKEYFNEIDFKFKETCEYKQFNEFAEDVNLQAYQISFIEVSKKYIKSLIDDNKIYLFKIYNKDFSKYSKGTPNLHTLYFKMLFDKENLENPIYKLSGNAEMFFRKGNLDLDKTTIHHANQPINNKNPNNRKKQSVFKYDIIKNRRYTVDKFALHMSITTNFQVYKNKNVNETVNRALKYCDDIYAIGIDRGERNLLYACVVNSRGEIVKQVPLNFVCNTDYHQLLAKREEERMNSRKNWKIIDNIKNLKEGYLSQAIHIITDFMVEYNAVLVLEDLNFRFKEKRMKFEKSVYQKFEKMLIDKLNFLVDKKLDKNANGGLFNAYQLTEKFTSFKDMKNQNGIVFYIPAWMTSKIDPVTGFTNLFYIKYESIEKSKEFFGKFKSIKFNKVDNYFEFEFDYNDFTDRAQGTRSKWTVCSFGPRIEGFRNPEKNNKWDGREIDITEKIKKLLDDYNISLDEDIQAQIMDINTKDFFEKLIKYFKLVLQMRNSKTGTDIDYIISPVRNKQNEFFDSRKKNEKLPMDADANGAYNIARKGLMFIDIIKETEDKDLKMPKLFIKNKDWLNYVQKSDL

SEQ ID NO: 2 - protein, 1188 aa, unidentified
MNIYEKFTNMYQVNKTIRMGLKPICKTDENIAKFLEEDKERSEKYKIAKKIIDKENRAFIEDRLKDFSISGLDEYLELLKQKKDITKIQKKMRDEISKQLKGFPQFDSKYKFQYITDKEDTEILEYFKKFTTFFTGFNSNRENVYSKEDISTSIGHRIIHENLPKFISNFRILNKAIEALGTGKINEDFKNNEINVTVEELNKIDYFNKVLTQSGIDLYNNLIGILNQNINLYNQQQKVKKNKIGKLETLHKQILSEKDKVSFIEEFAEDNQLLKCIDEYFKEKSCLINVDLKNLLENIDTYSLNGIFIKNDKSLKNISIYLYKDWGYISNLINEEYDYKHKNKVKDDKYYEKRKKAIDKIKYFSIGYIDELLKDKNVPMVECYFKEKINLVVKEFNASLNKFNEYKFTNELKTDEIAVEIIKNLCDSIKKIQGIIKPLIITGNDKDDDFYVEINYIWDELNKFDKIYNMVRNYLTKKDYIEEKIRMMFSKSSFMDGWGKDYGTKEAHIVYHDKNYYLVIVDEKLKLEDIDKLYKPGGDTVHYIYNYQSIDYRNIPRKFIYSKGNRFAPSVERYNLPIEDVIEVYNNKYYRTEYEEKNPKIYKKSLTSLIDYFKIGVNRDMDFEKFDIKLKDSNEYKNINEFYYNLETCCYKLQEEKVNFSVLEEFSYSGKIYLFKIYNKDFSKYSKGTPNLHTLYFKMLFDKENLENPIYKLSGNAEMFFRKGNLDLDKTTIHHANQPINNKNPNNRKRQSVFKYDIIKNRRYTVDKFALHMSITTNFQVYKNKNVNETVNRALKYCDDIYAIGIDRGERNLLYACVVNSRGEIVKQVPLNFVGNTDYHQLLAKREEERMNSRKNWKIIDNIKNLKEGYLSQAIHIITDFMVEYNAVLVLEDLNFRFKEKRMKFEKSVYQKFEKMLIDKLNFLVDKKLDKNANGGLFNAYQLTEKFTSFKDMKNQNGIVFYIPAWMTSKIDPVTGFTNLFYIKYESIEKAKEFFGKFKSIKFNKVDNYFEFEFDYNDFTDRAQGTRSKWTVCSFGPRIEGFRNPEKNNNWDGREIDITEEIKKLLDDYKVSLDEDIKAQIMDINTKDFFEKLIKYFKLVLQMRNSKTGTDIDYIISPVRNKQNEFFDSRKKNEKLPMDADANGAYNIARKGLMFIDIIKETEDKDLKMPKLFIKNKDWLNYVQKSDL

SEQ ID NO: 3 - protein, 1198 aa, unidentified
MTSKNSFEDFTNLYELSKTLRFELKPVGETLNSLEKDGVIQKDKEVEENYNKIKVFFDSLHREFVKQSLEKGYLNEIDNFYNSYIELNKTPENKKNKTLQKDFEKASKILKKELVSFFESKGNEWKQKYSFFKKGGIDVLNEKEVLDLMGEFYPEQKELFKKFDRFFTYFSNFKESRKNFYSEDGRAGAVATRAIDENLITFIKNLEDFKRFQNNFSDFISQNFSQEEKQAFELDFYNSCLLQDGIDNYNKILGGYSLENGNKIQGINEKINLFKQNQNKSNSKDIKFPRFKLLYKQILSDKEKIAFIDEIESDEKLLDFVKNSLEKNKFKIIEAEKIINNFIKNNPSFELDKIYLSKVSINTISNKFFASWDYILKEGFEKGEIKDFVSFEELKNAFEKIKYEKLEDIFKANYISENIVLEGESLYKNFLHIFFNEFQKNISDINLYTSKIEKLLGEKFEKTEIQVETIKNYFDSVLSLYKMTKYFALEKGKSKVDLETDNNFYNDFFAYYEDFEIWKNYNLVRNFVTKKQIQTDKFKLNFNNSQFLTGWDKDKEKERLGIILKKEDKFYLGVFKNNKILDNYKYESGDFYEKMNYKQLNNVYRQLPRFAFSNSKKEVYGITPELKKIKEEFDIFQKSKEKGEKFDPEKLKKIIECYKRGFIATYENEFDLEKIKNTDYTDLPTFYDDIEKKTYKLQFSKINSDFIEEKVKSSDLYLFQIYNKDFSEFKKAGSKENIHTKYFKLLFDKKNLENLVIKLSGGAEIFFREKTEKLKTKLDKSGKEVLDHRRYGQDKTMLHLSITLNANTGDSYGFNKMINEYLNKNEDIKIIGIDRGEKHLAYYSVIDKTGKIYDIGSLNEINGVNYLVKLEKIELSRKDARVSWGEIENIKELKNGYISQVINKLAELITKHNAVIVFEDLNQGFKRGRQKIEKQIYQKLELALAKKLNYLTQKDKGDDEVLGNLKALQLVPKVNDYQDIANYKQSGIMFYTRANYTSTTCPCCGFRKNIYISNSDTKEKQIKAFEKINIKFDGKKFIFSYEMEQDKKTKQKSNKTSFIVNSNFSRFKYNTLTKEIDNINITEELKRIFENIDLKGNINEQIKVLKGFSFSELTKKFNLLLQLRNSDTKNALDYIICPACNYHSKDGFQGLDYNADANGAYNIARKGIIMIERIKNIFEKPDLYVSDIDWDNFTQKTQ

SEQ ID NO: 4 - protein, 1193 aa, unidentified
MKTFENFTNLYSLPRTLRFELKPLYKTKELIDSKQELFPKDKRIDEIYQNIIKPCLNELHSDFIEKSMENKDFQNIPDNILKIYSNEKNIDDFKNIEKDLIKQINGFLKSNKTFFAENYSDLLGKNSIDIIIKVFGEKIYKKDDSGKIFLYNDLLGKSYEELINIYFKGFSTYLSNFNKNRENLYDKKNEAKVGSVSGRTIGENFPRFLQNCINFRDKLEKLNLSIEQKDIFITNNFGKCISQKQIDYYNKIIGQINSKTNEFNQKNGLKGNKKLPKLLLLHKQILGKSENENILNFINNIIQTDFELEQEIKIINKDIFQRIDFIKKSIVSNIEDFELEKIFIKKNRLKDISSLLMDNYSVLEKLLPEFNEEGKIIKENELVNLSKIKKSFENIDLKDLKNIFKKEYFDESKDGFKLFLNGIYNHFSDLENNIKHTHKLVQDKLISGNFSENIQKSEKNINLRDEIFVSSKGLLKAYLDSILALDRFVHMFDYGEQKDFDSNFYNNIEEYSINFSPFKTYNAVRNYLTKKNYSTDKIKLNFDYPDFLGSNSLGKYAFIYKDSKGFYYLGVLDHSNSQSKYKPQILKNNTEFYQLEYKQIKFNTLAGKGYIRDFGVKYSEDENCIINLKTLIKKQYLERYPVLKEIVDFQTDDKKIFDAKVKTILEQAYSINFVNIDKNYILEENNNGNLHFFQIYNKDFSENKKINSMENLHTMYFKALFEKENFNGGACFKLNSQGAEIFFREKSINEKKVKDLKTRNENAIEKKRYTENKVFLHLPITLNFINKGYSKYSFGYINDSVKKYIKENKISIIGIDRGEKNLIYFSMINENLEIIELKSLNSLILKVSDLEEKEVNYFEKLSKKEGNRNKERKDWDEIETIKELKEGYISQIVDNLVKLIVKHNAIVVMEDLNSGFKRGRQKIEKQIYQKFELALAKKLNFTVDKNKKHDELGGIYKAYQLTPQIENFQDIYSQTGIIFYTQAAYTSVTCPNCSFRKNIYQKYENESKFKEFFKKYILEIKFEDNCFIIKYKIDEKIDKKKNKLKKLEFQVNTKNQIRLKFEKSLKGKGGETKEFNITEKFKEIFEKHKLDLGNLKEELLAWNGEIQLYKDFMFYFNLLLQLRNSKENDNGGYISCPSCGFHSGNGFQGFSYNGDANGAYNIARKGRIILDKIKKDEKNLGITNVEWDNYYQK

SEQ ID NO: 5 - protein, 1221 aa, unidentified
MEKFYDKFNNLFSVSKTLRFELEPIGKTKEFFEKYILKEDERKSDLFKKVKKYCDEYHKLFISECLKNFDDKDFENLLKEYFALVNLSNPSEKETKRITDILKVLRAKISDRFTKNPKYKGLFGKELINDYLKDYYKNNKNILDEITPFKDFTSYFTGFNTNRKNMYSKEEKHTAIAYRLIDENLSTYIKNIKAFNIILENIPDIKIQIKENLNLDCDEFFSNIENYTNVLTQEQIETYNLAISGKSQENSKIKGINEIVNLYKQKNKIKIPKLKELYKQLLSDTTSASFKFDIIEYDYQIVEMINSYYETFKKNFIDDNGCQSALSNIQNYDIKNIYINNDVSLTSLSQNIYNDWNYINSLLGNEYDQNYKGKAKFGTEKYSEKKEETLKKIKEISLLKIEELIKNYDKENNNSEKIFEYFKNAIEENLKKIDIEYKNTKTILQKEYDKTSTDLLKDEQSIEKIKNFLDSIKDLQILIKYLIPKNNTLETDLEFYNALNYDILSEIIPVYNKTRNYLTKKTYSLEKFKLNFDCPTFLNGWDLNKEEANLGTLFRKDGNYYIGIMNTNSRKSFIEYNQAEFKNEKKYQKIEYKLLPNPNQMLPKVFFSESRIKEFNPSEELLSKYERGYHKKGADFDLNFCHQLIDFFKSSINKHEDWSKFNFKFSETNKYNDISEFYKEVELQGYKISFTDISEEYINKLVEEGKLYLFRIYNKDFSSYSKGTPNLHTLYWKALFDEDNLKNIIYKLNGGAEIFYRKKSIEAKITHPKNQPIDNKNPNNPKKQSIFEYDLIKDKRFTLDKFQFHVPITLNFKAKDTHKLNDLVNNKIKNTEDINIIGIDRGERHLLYACVINKNGEILEQYSLNTIGQTNYHYLLSKKEDDRLKQRQAWGTIQNIKELKEGYMSQVIHKLTKLMVQYNAVIVLEDLNSGFKNSRKKVEKSVYDKFETALINKLSYLVDKSIQNKFEQGGIMNAYQLANADIKSAKQNGVIFYIPAWCTSKIDPTTGFVNLFDLKKIDKEFVKKFNSIKFNNTENYFEFDFDYSNFSDKSSGDRTNWTLCSFGNRIRTFKNPKKNNNYDSQTVELTQEFKKLFDCYNLDCSNLRDEILNKSDSKFFNATPEKNNFYGFALLFKLLLQLRNSITGSEEDYILSPIKNKKGLFYDSRTANGVLPENADANGAYNIARKGLLILNRIKNSEENTKTDYTIKNADWLTYVQQQDR

SEQ ID NO: 6 - protein, 1224 aa, unidentified
MQLNPVGKTMDYFQAKQILENDEKLKENYQKIKEIADRFYRNLNEDVLSKTGLDKLKDYAEIYYHCNTDAERKRLDECASELRKEIVKNFKNRDEYNKLFNKKMIEIVLPQHLKNEDEKEVVASFKNFTTYFTGFFTNRKNMYSDGEESTAIAYRCINENLPKHLDNVKAFEKAISKLSKNAIDDLDATYSGLCGTNLYDVFTVDYFNFLLPQSGITEYNKIIGGYTTSDGTKVKGINEYINLYNQQVSKRDKIPNLKILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIREKSIVNYYKTSLMQLTDNLSDKYNEAAPLLNKSYANEKGLKNDDKSISLIKNFLDAIKEIEKFIKPLSETNITGEKNDLFYSQFTPLLDNISRIDILYDKVRNYVTQKPFSTDKIKLNFGNSQLLNGWDRNKEKDCGAVWLCRDEKYYLAIIDKSNNSILENIDFQDCDENDCYEKIIYKLLPGPNKMLPKVFFSEKCKKLLSPSDEILKIRKNGTFKKGDKFSLDDCHKLIDFYKESFKKYPNWLIYNFKFKNTNEYNDIREFYNDVASQGYNISKMKIPTSFIDKLVDEGKIYLFQLYNKDFSPHSKGTPNLHTLYFKMLFDERNLEDVVYKLNGEAEMFYRPASIKYDKPTHPKNTPIKNKNTLNDKKTSTFPYDLIKDKRYTKWQFSLHFPITMNFKAPDRAMINDDVRNLLKSCNNNFIIGIDRGERNLLYVSVIDSNGAIIYQHSLNIIGNKFKGKTYETNYREKLATREKERTEQRRNWKAIESIKELKEGYISQAVHVICQLVVKYDAIIVMEKLTDGFKRGRTKFEKQVYQKFEKMLIDKLNYYVDKKLDPDEGGGLLHAYQLTNKLESFDKLGMQSGFIFYVRPDFTSKIDPVTGFVNLLYPRYENIDKAKDMISRFDDIGYNAGEDFFEFDIDYDKFPKTASDYRKRWTICTNGERIEAFRNPAKNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKSDNVSTVGPVIHNDKWLKFVQENDMANN

SEQ ID NO: 7 - protein, 1257 aa, unidentified
MNQNTSFSQFIGLYPVSKTLRFELKPIGKTLENVQNKGIIEQDEAKSLKYVEAKQIIDQYHKYFIGEALEGVNLDWNPLQKAFEDYLTNRNAPAKKNLEKTQESFRKEIAKKLSEHKHFKELTAATPKELFEKILPEHNGKQEAIEAFAKFSTYFKGFQENRENIYSSDAISTGVPFRIVHDNFPKFLSSIQVFQNIKENCPCVLDDAEKELQELLKSQKLIDVFNINYFNNVISQDGIDFFNQVIGGYTTPNGTKVRGINEFANLYRQQNPEFAKKHIATKMIPLYKQILSDRESMSFVLESFNKDTDVQDGVKEFFEHHLLRCNMDGNEINVLEKIGELILGLKNYDSLNKFFVSRDSLSSISQKLFDNWNTLNNYLFNYWENKIGSVEKASNKKRIDAKLKEDYFSIEELNEVLKFNESDANICDFWTDYTSQANSVLSGEIKKNYEEFLAADYSNKALRENEADVQKLKDFLDAIVNILHLVKPLMMDSDQGEPSFYNELNPLYDQLSSIIPLYNKTRNYITQKISDSTKKFKLNFENPTLADGWDQNKEEANTSIILKKNEKFYLGIMNAKDKPKIGLYKVKPEEPHYDKMVYKLLPGPNKMLPKVFFSAKGKEIYKPSKEIQDGYAAGKHKKGDSFDKQFCHHLIDFFKEGISNHPDWKNFNFKFSETSSYEDISAFYNEVSDQGYKLSFFPIPDSQIDAWVNEGKLFLFQIYNKDFAPGAKGKPNLHTLYWKATFSLENLKDIVFKLNGEAELFYRRSSIKKPYSHKIGEKMVNRITKDGRPIPDAIFGELFRYFNNSSKTSLSDEAKKYLDFVTVKDVKHEIIKDKRYTEDKFEFHVPITINFKADDGSRYINDQVKDFLKNNSDVNIIGIDRGERNLIYMTLINQKGEILAQKSFNLVGNTNYHEKLSIREQERDAARRSWQSIGKIKELKEGYLSLVIHEIAKTMIENNAIIVLEDLNFGFKRGRFCVEKQVYQKFEKMLIDKLNYLVFKDCADSEYGGILKGYQLTQKFTSFKDIGKQNGFLFYVPAAYTSKIDPTTGFVNLFNFRDLTNAEKKKDFLTTLDDITFNSETNSFAFTFDYSKFKVFQTDYQKIWTVFTNGERIVYDRESKKHNIIEPTTIIQEALEKQGIQCVDQLNVLAEIEKIEPTRENARLFDSICYAFEKTLQMRNSNSETGDDYILSPVKNKNGVFFNSNEAGDKLPKDADANGAFHIALKGLFLLQHISETDEKLKIPHEKWFEFVQSRNK

SEQ ID NO: 8 - protein, 1300 aa, unidentified
MNFQPFFQKFVHLYPISKTLRFELIPQGATQKFISEKQVLLQDEIRARKYPEMKQAIDGYHKDFIQRALSNIDSQVFEQALNTFEDLFLRSQAERATDAYKKDFETAQTKLRELIVHSFEKGEFKQEYKSLFDKNLITNLLKPWVEQQNQIGDSNYTYHEDFNKFTTYFLGFHENRKNIYSKDPHKTALAYRLIHENLPKFLENNKILLKIQNDHPSLWEQLQTLNQTMPQLFDGWDFSQLMQVSFFSNTLTQTGIDQYNTIIGGISEGENRQKIQGINELINLYNQKQDKKNRVAKLKQLYKQILSDRSTLSFLPEKFVDDTELYHAINMFYLEHLHHQSMINGHSYTLLERVQLLINELANYDLSKVYLAPNQLSTVSHQMFGDFGYIGRALNYYYMQVIQPDYEQLLASAKTTKKIEATEKLKTIFLDTPQSLVVIQAAIDEYIQLQPSTKPHTQLTDFIISLLKQYETVADDQSIKVINVFSDIEGKYSCIKGLVNTKSESKREVLQDEKLATDIKAFMDAVNNVIKLLKPFSLNEKLVASVEKDARFYSDFEEIYQSLLIFVPLYNKVRNYITQKPYSTEKFKLNFNKPTLLSGWDANKEADNLSILLRKNGNYYLAIMDTAKGANKAFEPKTLNQLKVDDTTDCYEKMVYKLLSGPSKMFPKAFKAKNNEGNYYPTPELLTSYNNNEHLKNDKNFTLASLHAYIDWCKEYINRNPSWHQFNFKFSPTQSFQDISQFYSEVSSQSYKVHFQTIPSDYIDQLVAEGKLYLFQIYNKDFSPNAKGKENLHTLYFKALFSDENLKQPVFKLSGEAEMFYRPASLQLANTTIHKAGEPMAAKNPLTPNATRTLAYDIIKDRRFTTDKYLLHVPISLNFHAQESMSIKKHNDLVRQMIKHNHQDLHVIGIDRGEKHLLYVSVIDLKGNIVYQESLNSIKSEAQNFETPYHQLLQHREEGRAQARTAWGKIENIKELKDGYLSQVVHRIQQLILKYNAIVMLEDLNFGFKRGRFKIEKQIYQKFEKALIHKLNYVVDKSTQADELGGVRKAYQLTAPFESFEKLGKQSGVLFYVPAWNTSKIDPVTGFVDLLKPKYENLDKAQAFFNAFDSIHYNAQKNYFEFKVNLKQFAGLKAQAAQAEWTICSYGDERHVYQKKNAQQGETVIVNVTEELKVLFAKNNIEVAQSVELKETICTQTQVDFFKRLMWLLQVLLALRYSSSKDKLDYILSPVANAQGEFFDSRHASVQLPQDSDANGAYHIALKGLWVIEQLKAADNLDKVKLAISNDDWLHFAQQKPYLA

SEQ ID NO: 9 - protein, 1205 aa, unidentified
MKLEDFTNLYSLSKTLRFELRPIGKTRENIENGGLLRQDEDRAEKYVHIKKLIDEYHKAYIDKQLSGLVLQYADIGKANSLEEYYHSTRKSKDSDKDKIVKIQDNLRKQIVKRLKDSDEFKRIDKKELIQSDLAEFIKPAEDRALIAEFKNFTTYFTGFNENRQNMYSDKAISTAIAYRLIHENLPKFIDNIETFDRIAGITELYDQTSSDAEIFRLAHFSETLSQKQIDAYNSVMGRYNMLINEYNQTHKQSRLPKFKMLYKQILSDREHPSWLPEQFESDTAVLTAIRECYDDLRIPMANLKTLLEGLGNYDPSGIFLRNDQHLSQISKRLTGDRSSIERSVTEDLLTSRKLNKRKSRTTDEEESRKLFKQKGSLSIGYIADTAKIDVERYFAKLGAINTVTEQSENLFAKAENARTTADELLANDYPAGKRLVQSNDDIALVKNLLDALMELQWFVKPLLGTGDEAGKDERFYGEFTQIWEQLDRITPLYNMVRNYVTRKPYSTDKFKLNFESAALLGGWDKNKEPDCLSVILRKDEQYYLGIINKNHKKIFARDILPCEGECYEKMVYKQISISAGIGGFFRKCYRTAEQYGWCCSDSCLNDERKIIIRDDEARENLTEIINCQKDFLNKYEKDGFKYRDFGFRFAASSDYEKLSDFYRDIDEQRYKLSFTPVSATYIDSLVEEGKLYLFQIYNKDFSAYSKGTPNMHTLYWRMLFDERNLSDVVYQLNGGAELFFRRKSLQNGRPTHPANIPIKNKNSRNDKKESLFDYDLIKDRRYTVDKFQFHVPITLNFKSDGAGRINERVREYLRSADDVHVIGIDRGERNLLYLVVTDMDGNICEQFSLNEICNTDYHSLLDEREHKRMQERQSWQAIEGIKELKEGYLSQVVHRIATLMVKYRAIVVLEDLNFGFMRSRQKVEKSVYQKFEHMLIDKLNYLVDKKANPTTPGGLLKAYQLTDKFESFQKLGKQSGFLFYVPAWNTSKIDPATGFVNMLDLGYESIDKAKALLCKFDSIRYNACKDWFEFALDYDKFGSKATGTRTKWTVCTYGQRIDTYRNKDSQWVSRDVDLTNELKSLFSEHGIDIYSNLKDAIVAQNDKEFFANMLRILKLTMQMRNSKTGTDIDYIVSPVADANGRFFDSRQADATMPKDADANGAYNIARKGIMLVQQIKQSDDLRTMKFDISNKSWLRFVQHTNQADE

SEQ ID NO: 10 - protein, 1224 aa, unidentified
MSKIEKFIGKYKISKTLRFRAVPVGKTQDNIEKKGILEKDKKRSEDYEKVKAYLDSLHRDFIENTLKKVKLNELNEYACLFFSGTKDDGDKKKMEKLEEKMRKTISNEFCNDEMYKKIFSEKILSENNEEDVSDIVSSYKGFFTSLNGYVNNRKNLYVSDAKPTSIAYRCINENLPKFLRNVECYKKVVQVIPKEQIEYMSNNLNLSPYRIEDCFNIDFFEFCLSQGGIDLYNTFIGGYSKKDGTKVQGINEIVNLYNQKNKKDKEKYKLPQFTPLFKQILSDRDTKSFSIEKLENIYEVVELVKKSYSDEMFDDIETVFSNLNYYDASGIYVKNGPAITHISMNLTKDWATIRNNWNYEYDEKHSTKKNKNIEKYEDTRNTMYKKIDSFTLEYISRLVGKDIDELVKYFENEVANFVMDIKKTYSKLTPLFDRCQKENFDISEDEVNDIKGYLDNVKLLESFMKSFTINGKENNIDYVFYGKFTDDYDKLHEFDHIYNKVRNYITTSRKPYKLDKYKLYFDNPQLLGGWDINKEKDYRTVMLTKDGKYYFAIIDKGEHPFDNIPKDYFDNNGYYKKIIYRQIPNAAKYLSSKQIVPQNPPEEVKRILDKKKADSKSLTEEEKNIFIDYIKSDFLKNYKLLFDKNNNPYFNFAFRESSTYESLNEFFEDVERQAYSVRYENLPADYIDNLVNEGKIYLFEIYSKDFSEYSKGTNNLHTMYFKALFDNDNLKNTVFKLSGNAELFIRPASIKKDELVIHPKNQLLQNKNPLNPKKQSIFDYDLVKDKRFFENQYMLHISIEINKNERDAKKIKNINEMVRKELKDSDDNYIIGIDRGERNLLYVCVINSAGKIVEQMSLNEIINEYNGIKHTVDYQGLLDKCEKERNAQRQSWKSIENIKELKDGYISQVVHKLCQLVEKYDAIIAMENLNGGFKRGRTKFEKQVYQKFENKLINKMEYMADKKRKTTENGGILRGYQLTNGCINNSYQNGFIFYVPAWLTSKIDPTTGFVDLLKPKYTNVEEAHLWINKFNSITYDKKLDMFAFNINYSQFPRADIDYRKIWTFYTNGYRIETFRNSEKNNEFDWKEVHLTSVIKKLLEEYQINYISGKNIIDDLIQIKDKPFWNSFIKYIRLTLQMRNSITGRTDVDYIISPVINNEGTFYDSRKDLDEITLPQDADANGAYNIARKALWIIEKLKESPDEELNKVKLAITQREWLEYAQINI

SEQ ID NO: 11 - protein, 1226 aa, unidentified
MKNFFQLFTNQYELSKTLRFELRPIENTAELLKKNKIFEKDKKVYEYYQKTKKYFDKLHCEFIDEALVNTSLPNKDYSIYDKLFFEYKKDKENKKIYKALNEHAKKLRTNIVKDFEKTAEKWRDEYILSIKDEKSQKKLKQLEGLDLLFKTEVFDFLKKKYPEAQIEGNSIFDSFNKFFTYFDNFHQSRKNFYKDDGTASAIPTRIIDDNLPKFLENKKIFEEEYKVLNLSREEKDLFRLDFYNKCFTQTGIENYNEIVSKINSKVNKYRQKNKKSKIHFMKELFKQILSKRSKQATEQDTSISIENDSQVFEVLQDFIMETKNCNRMAKNILETFIQHQEEFDLQKVFLKGSNVTRISDMWFTSWFAFGDLLPKNSTGKKLANFISFQDIKNALEKTEIQDVFKKKYAKYNKESFYDQFLQIFDYEFHKALNDCEKNIVEAEKMMKVDKVYSNKKGILRNGKNGEKQKEIIKDFADSALSIYQMMKYFALEKGKKPVTDMDEDPRFYNDYKDWCEKSRTLLYYNEFRNYLTKKPYAEDKIKLNFEKSTLLMGWSANEEGNLQYYSSILRKDNKYYLGLMSSASTFNINKKEAFETRDGFYELMNYRQLKSTTIYGSLYKGEYKKEYRKDKETLTNQELIERTKKILEKQKFSYPQLEVILDKKYKAAEDLAKDLGNIVLYNIGFVKISKRYVNNLTNENFYLFEISNKDFLNKKKKGNVNLHTKYFQLLFDKKNLESKKGAILKLSGGGEVFYRKATKNLPKKKDKKGKEIIDRKRYAEDKILFHLPIVLNIGFKDKQINSKVNQVLATQRKARIIGIDRGEKHLLYISVIDENANILKIKSLNTIDVPNKKEPDDYHKLLDEKEKERDDERKSWHTIENIKELKHGYISQVVNEIDKIIFECLDERILPIIVFEDLNIGFKRGRFKIEKQVYQKFELALAKKLNYLVSKEKGNYLNAFQFTPPVNNFQDIHKKQVGIIFYIPASFTSAICPVCGFRKRLYGFTFKNINQVKKLLAEHEFNIFYDGKRFNFECLASKENEKKENKNGLYTEIFLNKKLFFNSDIERLFNKKSKDNKKWETISFIATEELKKLFEGKIDLKRNIMDQILAGDFRAEFYQQLIFIINRILNLRNCHAVEHRDFIACPSCGFHSEKNYEKLKSRYAGKEKFEFNGDANGAYNIARKGILLIKKIRQFAKSNNIEKLMPYDHLQIDMQEWDKFCAKNKS

SEQ ID NO: 12 - protein, 1226 aa, unidentified
MFNLYSNLRKLYPVQKTLRFELIPQGKTKKNMEKEGIIREDEHRAKIYNKVKKYCDEYHKIFIEDCLKDIKLDNLNKYYELYSIDKKDEDQKEEFTKTQEKLRKQISESFKNNNKYKGLFQKDIIDTYLINMYKEDKQKLQDINEFNKFTTYFTGYNKNRENMYLEEEKSTAIAYRLINENLPTFVDNLKIYNKLLKFIPEIIEKIHIELAEYIQVNSIDEIFDINYYNDVLTQKGIECYNIIISGKTKDEGTKIKGINEYINEYNQTHTNKIPKLRELYKQILSDIDTASFKFDVIEDDIQLLDSINLYYQQIFPIISKVNELFSKINNYNLDLIYINNDGTLSNVSNEIYSDWSYIRNHISEKYDTEYTGRLKVGTEQYSKQKQEYLRKQRNYSLSFLINCMKDDKIIEYLSNNMQQTGIVEKIKMDFDNFKSIQIQEDYKCLIKSENSIAKIKKLLDDTKLLQVFMKTLIPKDKTIEKDNEFYSELISYYDVFRDIIPLYNKTRNYLTQKPYSNEKIKINFACPTLLNGWDLNKEEANLGVILLKDEKYYLGIMNPYCKKIFNVDNRDSDTNNNYKKMEYKLLPGPNKMLPKVFFSKSRINEFAPSDELLEKYNKGYHKKGKDFDINFCHELIDFYKKSLNKHEDWKNFNFKFKNTVEYNDISEFYREVEEQGYKIEYKEYSEKYINELVERGELYLFQIYNKDFSEYSKGKQNLHTMYWKAVFDPENIKDTIYKLNGNAEIFYRKKSIDEKITHPANIPVANKNENTIKAGKITSLFKYNLIKDKRYTVDKFQFHVPITMNFKSEKILNINQIVNKYLKYNDDIHVIGIDRGERNLLYVCVIDKDEKIVYQKSLNEIYNEYQGIQYSTNYHKLLNEKENEREQAREDWKNIENIKELKEGYMSQIIHILCELMRKYNAIIVIEDLNKGFKNSRIKVEKQIYQKFEQMFINKLNYVVFKDVPKLEEGGILNAYQLTNKFESFTKLGKQSGILYYIPAWCTSKIDPTTGFINRFYIKYENLEKSREFVNRMDNIRYNLDEELFEFDIDYSKFSDRLNDTKNKWTLCSYGERVYTHKNNKEIWVDDKIQLTDEFIKLFEKYKIDLNNIKNEIIEKADIEFFKGGENSLGFLQLFKLMVQMRNSITGQEEDNLISPVRNNEGIFFDTKAKIKGLPEDADANGAYNIARKGLMLIEQMKRTDDDKLNKIKYDITEKEWLNYIQNRGV

SEQ ID NO: 13 - protein, 1226 aa, unidentified
MSNLYRNLHNFYSVQKTLRFELIPQGKTKENMEKEGILKADEHRAEIYSKVKKYCDEYHKLFIDKCLKNIRLNELNKYYELYSVVKKDEKQKEEFIKIQEKMRKQISESFKNDNEYKGLFQKDIINIYLITMYKDDKEKIKDISEFNKFTTYFSGYNKNRENMYSEEEKPTGIAYRLINENLPTFIENFKIYNKVIKFMPEIINKIHTDLMEYIQVEDIDEIFDINYYNEVLTQKGIECYNIIISGKSKSNGEKIKGLNEYINEFNQKHNEKIPKLQELYKQILSDTDTASFKFDTIESDEELLNNIESYYTKLLPVFNKINQLFAKFNKYNLDLIFINNDGTLNTISNEIYKDWSYIRNRIGERYDIEYTGKLKKDTEQYSKQKQEYMKKQKQYSLKFLNDSLRDNYLIEYISNYIEQSKIMEKMKTDFTEVQKIESRGDTKQLIKDENSIVKIKNLLDDIKFLQEFVKILVLKDRTIEKDAEFYSELMPYYNELKDIIPLYNKTRNYLTQKPYSTEKIKLNFECPTLLNGWDLNKEEANLGVILLKNEKYYLGIINPYCKKIFKIQEKDSNSENNYKKMEYKLLPGPNKMLPKVFFSKSKIDKFMPSDELLEKYNKGCHKKGKDFDINFCHELIDFYKTSLNKHEDWKKFDFKFKSTSEYNDISEFYKDVEEQGYKIEYSEYSEKYINELVDRGELYLFQIYNKDFSEYSKGRPNLHTMYWKAVFDIENIKNPVYKLNGEAEIFYRKKSLERKITHSANEPVANKNENTIKSGKPTSLFKYDLIKDKRYTVDKFQFHVPITMNFKSEKMFNINQVVNKYLKYNDDINVIGIDRGERNLLYVCVIDKNEKIVYQKSLNEIVNEYKSIKYSTNYHALLNKKEKEREIAREDWKNIENIKELKEGYMSQVIHILVELMRKYNAIIVIEDLNKGFKNSRIKVEKQVYQKFEKMFIDKLNYLVFKDEPKESEGGVLNAYQLTNKFETFNKIGKQSGVLYYIPAWCTSKIDPTTGFINRFYIKYENLDKSKEFINKIDDISYNSLENLFEFDIDYSKFTDRLNETRNKWTLYTNGERIYTYRNDKGEWIDKKIQLTNEFKKLFEKYSINLDNIKNEILEKANIEFFKGNNETLGFIQLFKLMVQMRNSLTGKEEDNLISPVKNSNGKFFNTNEQIEGLPKDADANGAYNIARKGLMLIEQMRNTEDDKLNKIKYNITEKEWLDYVQNRGM

SEQ ID NO: 14 - protein, 1226 aa, unidentified
MNIYKNLHKKYDLSKTLRFELKPIGNTLTNIEEGGFIKKDEQRAIDYKKVKKYCDEYHKVFIERVLSNYELPNELLIKYNELYEKENKDIDTRNEIREIEGKLRKDISEAFKSDEQYKGIFSKDMIQKYLKELFKEQDDKIKEISEFDKFTTYFAGFNKNRENLYIEDEASTSIAHRLINENLPTFITNMKIYKKFTKLFPELKDLISSNLKVNNVEEMFELEGYNKVLSQTGIEHYNLLIAGKSEEKTKIKGINEYINEFNQKNKEYKIPKLKPLYKQILSDKTSESFAFESIDDDSELTEQLEIYNKEFFDAINGNSGIIQLLANLKQYQLDKIYINNNLDLTRISQEIFGDWNRINEILIEDYDKNYTGKVKVNTEKYEDERKKKLKSIEEYSIESLDKKIKEYRDEENKMVDYLETYITLEKVKNIANKYSECQKYLINTSVGEKKLVKNESAINAIKDYLDSVKDIQSFLKMFIPKHKTIETDSNFYNIFSEKYEIIYNIISLYNKTRNYLTQKPYSIEKIKINFNNPILLNGWDLNKEKDCLGVILIKDNKYYLGIINPYNKKIFDIEETKDEENVYKKMEYKLLPGANKMLPKVFLSAKGIENFKPGSEIIEGYSKGLHKKGENFNIEFCHKLIDFFKDSINKNEDWNKFEFKFSPTESYNDISEFYKEIEKQGYNIKFKKYSAEYIEKLVEIGDLFLFEIYNKDFSKFSKGKPNLHTLYWKALFSEKNLENIIYKLNGQAEIFYRKASLNIADTAVHPKNQPIKNKNDLNEKKESIFTYDLIKNKRYTVDKFQFHVPITMNFISEKVNNINEAVNKELKMADHFNIIGIDRGERNLLYITVINEKGEILEQYSLNKIINEYNGKKYVTNYHDLLDAKEKERELARESWKTIENIKELKEGYLSQVIHKITELCRKYKAIIVIEDLNSGFKNSRIKVEKQVYQKFEKMLIDKLNYLIFKDEPEDAEGGVYNAYQLTNKFESFTKMGKQSGILFYIPAWCTSKIDPTTGFVNLFKIKYKNINDAKVFINKFDNIRYNEEDDLYEFDFNYKNFTNKPFGIKENWTICTYGNRIKTFRNKDKNSNWDNQEINLTEEFKKLFSEYNIDNTNLKEDILNQEEKAFFEDLLALIRLTLQMRNSKTGTDIDYMISPVRNKFGEFYNSNMCSEQWPKDADANGAFNIARKGLMLVKQIKDTDVSKLARVKFNITNKEWLEFVQTMEK

SEQ ID NO: 15 - protein, 1228 aa, unidentified
MRQKYDGFESCYGISKVIRTGLIPQGATLEHIEKKGLLLLEAKRAEDYTKVKKIIDRYHRKYIDNQLKDAELSGVEESYSLYCKTNRTEKENTDLKKDLEKLRKQVSNFLTSSAEFKRIDKKELIEKDLLNFVQGDEEEEELVKSFKGFTTYFKGFHENRKNMYSHEEKSTAIAYRVINQNMFRYFDNIRSISKFILANSEKNIAELESIYSDKLNGKTLKEFFSVDNYSFFISQSGIDTYNMIIGGYSVDERTKVKGINEYINLFNQENHEKFGTLKPLYKQILAERTTESFLPEKFENDKEVIESLAAFSHNMSDTLLEIMEVFGKIGEYDASGIYLKYDSLKDISTCVFSDWSAITNALWERYDVQYIGKKKKETKAYIEEKRKHFKNLKSVSADEINTAMGENVLIPYSDAAKSLFEKICVAEENIRELINLDYPDDKELSKDKAAVKDIKAYLDSVKALQHHMKPLLGNQDESGKNEFFYGDFSGLWDELDALTPLYNKVRNYVTKKPYSTEKIKLNFDNSTLLNGWDLNKEEANTAILMQREGFLYLGIMDKKHNKIFRNIENTGCEDCYQKMEYKLLPGANKMLPKVFFSKKGIESFKPPLEILEIYKNETFKKGDAFNIDDCHTIIDFYKKALNEHEDWKKFEFKFSKTKDYRDISEFYREVEAQGYKISFKAIPKKIIDEYVEKGWLYLFKIWNKDFSEYSKGTPNLHTLYLKMLFDPRNLENVVYKLNGEAEIFYRKASIQPKDIIKHPANEAIKNKNPLNKKQSSIFKYDLIKDRRYTVDKFEFHVPITLNFKARGKENINTEVREYIKRSKNLYVIGVDRGERNLLYVSVVDADGSIVEQYSLNDIVNEYKGVQYRTDYHALLDTKEKNRDKARKEWTEIQNIKELKEGYISQVVHKICELMMKYEAIIALEDLNGGFKNSRKKVEKQVYQKFEKMLIEKLNYLVDKKADVNEPSGLLHALQLTSKFEGFNKLFGQSGFVFYVPAWNTSNIDPCTGFTNLFFSKDLRYASVENSLEFWKKFDSVAYDDTKDIFSFTFDYNNFNQRAEEIKSKSKWTVYSYGDRLETFRNADLNQQYDSREVKLTEAFKELFKKYEIPHENCEIKENILNCKERDFHDKLLHLFKLTLQLRNSKTGTMIDYMTSPVMDIDGNFFDSRTASSSLPQDADANGAYNIARKGLWLIEKLKNTKDADLPKAKLAIKNAEWLAYAQRNENNG

SEQ ID NO: 16 - protein, 1228 aa, unidentified
MWEDFTKLYSLSKTLRFELVPTESTEQQLKTSGVIQADKVRHESFKLAKKVLDEIHRKFITDSLVDLQLDHKQLQVFAEYSLNPASKSDAAKQAELIKLRRSIYMAFEQRFTATQAGWKEAYDCEKFISSPKSQLALIETFKPELADKLGMPEQDLEDLQTRFNKFASYFQGYNKNRENIYTTKGHAVEISHRTIDQNLLKFYKNVQKYEKYYTDKDILGGEAAPHFKVENYTSYITQDEINAYNEAIRSFRSNINQARQGLDKKEKQSLPMFEPLFKQILGDNTKKVLFEKLEGKEEYAELTRKLQQDMPSWVTALDEHANTHIMIPANPATVWFNAKAVNAVLHKHTTSPELIKIYLPKEFTSSRRAKDEETAKQNSFVSLRQLFAALDQAQVATELSLLKNVDRADMGASEAVWLAFCEDIQVKIKDARETVGAFSKIIAFENKSEKETLKKALDSFNDLYRMYTYFELRYKNEPVTSFDTDPDFYELFEDPDDQDSLFDADKDKRFSHYYDLIRNYLTKKSYSLDKWKLNFDCSQLLTGWDVNKEPEKHGVILQNGENYHLAILNNESNKIFDRAKNKLLFQAEDNTAWQKMDYKLLPGPNKMLPKCLLPKSNRNKYGATDEVLDIYETGSFKKGDNFNLASLHKMIDFYKQAINRYEGWSMYEFTFRSTDQYQGIDQFYHDAESQGYRLGFVPINSVELQKMVDDGRIYLFRIQNKDLANPSSKTANMHTLYFRELFKSSSTIKLNGEAEIFFRPKTENLAASKTNRKTPQGTEVTELKRFRSDKIQLHLPVNLNYQTKSLINATFNQHTIERIKTNGVNAIIGIDRGEKHLAYVAIVDRSGKLLDEPISLNNITSKLPGGGKKVTPYFDLLDHREKERKENRQNWEAVRQIKDLKSGYIGHCIKQIVDLMIKHNAVVALENLNVGFKQGRSAIEKSVYNQLEQALLHKLQYVVDKSKPPQELFGAQNGVQLAPPSVVPKDTYNHMGFVFFVDPSYTSAVDPKTGYRQHFRLDDRINTKTFNTFIRDGFKDIEYKDSNLLFTFNWRQLASARNKIKKDVRAEKEAEISDKPWTITANIERLVYSKKADDGHGATVKVYPQVELLQLFESKNFDLRKNIKQQLLEHKPDAKFVGSFTYLFNIINKLRNSDAASDVDAIISPVYIDGFNSTKHKLGGYEWNGDANGAYNIARKGAILLDKLYEAKTLRDFKATVTRAEYDAYLTK

SEQ ID NO: 17 - protein, 1233 aa, unidentified
MKDLSTFISQYDVSKTLRFELIPTPETEKHIREKGILDADEKLAVSYKRMKETMDEFHKDFIDRALSTAQLRHIDEYCALYWADAEEKNSKKYADRLKAASAELRKEIVQCFDADDAFKKIDKKEFIKDELETWINEKHPMLYYDPDFKKFTTYFEGYRKNRMNLYSDEEKVGSVAYRLIGENLPKFLDNIRIYEKVKASKVAGKFSKLCKDMEQYMNGRSLDEMFSARAYPSTLTQTQIDQYNTVIGGRNDGSSKLQGLNEYINLYNQQHPDAKLPIFKMLYKQLLSDRISASWLPEAFDSAQALFDGVEAFAQEVLTDTVLNDLQTVLNELPERDAALIYVRKSSVNDISSRLFGYYSVFNDALDAMGENEKTGYISLARLQSAVDGYIPTLDKDEYESIVSEYAPNCVTACFFDNLHEYIENISDAYEALRPCFGVEHDENYQLSEKERTWLKAYLDGVIALLHFIKPLYIDKDFALEKDEVFYSDFMPLYEKLSQVTKLYDKVRSFASKKPYSTKKIKLNFGCSTLLSGWDVNKETQNLGVLLIKDGNYYLGIMDKANNKLFSDAPECQSEDCYQKVVYKLLQEPYKMLPKVFFSKSRINEFAPSEEIISIRINETFKKGPKFIAADCHKLVDFYKTSISKHPDWRRFEFKFSPTGTYVDINDFYQEVAEQGYKLFFKPVSTAYIDSAVSEGKLYLFRIYNKDFSPSAKGKPNLHTMYWKALFDEKNLADVVYKLSGHGEVFYRSRSIDDEKRVIHPANKPIDKRNSGMSGETSVFDYDIIKDRRYTVDKFQFHVPISLNFKAHGVSNFNEKVCTYLKNNPDVNIIGIDRGERNLLYISMIDRSGRVVKDSRGNYIQYSLNTITGEYKNADGAPVKFSTPYHELLDKKEKNRREARENWGDIESIKELKAGYMSQVVYHISRLMVEYNAIVVMEDLNSGFKNGRKKVEKQVYQNFEKALIEKLNYLVFKDYPVDTCGGLYRALQLTDKFKSFGMLTKQTGFLFYVPAWNTSKIDPVTGFVDMLKPKYKSIPDARVFFSKFNYIRYNDQKDWFEFSFDYGNFTDRADGTKTDWTLCTYGSLRYAYNKSLNNGHGGYEKWNVTDKLKALFSANGIEYADGKDLKTKITAQNKADFYAILVKCLQVTLAMRYSSVEDGKDFILSPVADENGVFFCSEGRNDGLPQDADANGAYNIARKGLCVLEHIDVAEKYSDWSTKISNKEWLNFVQTRA

SEQ ID NO: 18 - protein, 1197 aa, unidentified
MLSNFTNQYQLSKTLRFELKPVGDTLKHIEKSGLIAQDEIRSQEYQEVKTIIDKYHKAFIDEALQNVVLSNLEEYEALFFERNRDEKAFEKLQAVLRKEIVAHFKQHPQYKTLFKKELIKADLKNWQELSDAEKELVSHFDNFTTYFTGFHENRANMYTDEAKHSSIAYRIIHENLPIFLTNKKLFETIKQKAPHLAQETQDALLEYLSGAIVEDMFELSYFNHLLSQTHIDLYNQMIGGVKQDSLKIQGLNEKINLYRQANGLSKRELPNLKPLHKQILSDRETLSWLPESFESDEELMQGVQAYFESEVLAFECCDGKVNLLEKLPELLHQTQDYDFSKVYFKNDLALTAASQAIFKDYRIIKEALWEVNKPKKSKDLVADEEKFFNKKNSYFSIEQIDGALNSAQLSANMMHYFQSESTKVIEQIQLTYNDWKRNSSNKELLKAFLDALLSYQRLLKPLNAPNDLEKDVAFYAYFDAYFTSLCGVVKLYDKVRNFMTKKPYSLEKFKLNFENSTLLDGWDVNKESDNTAILFRKEGLYYLGIMNKKYNKVFRNISSSQDEGYQKIDYKLLPGANKMLPKVFFSDKNKEYFKPNAKLLERYKAGEHKKGDNFDLDFCHELIDFFKTSIEKHQDWKHFAYQFSPTESYEDLSGFYREVEQQGYKISYKNIAASFIDTLVAEGKLYFFQIYNKDFSPYSKGTPNMHTLYWRALFDEKNLADVIYKLNGQAEIFFRKKSIEYSQEKLQKGHHHEMLKDKFAYPIIKDRRFAFDKFQFHVPITLNFKAEGNENITPKTFEYIRSNPDNIKVIGIDRGERHLLYLSLIDAEGKIVEQFTLNQIINSYNGKDHVIDYHAKLDAKEKDRDKARKEWGTVENIKELKEGYLSHVIHKIATLIIEHGAVVAMEDLNFGFKRGRFKVEKQVYQKFEKALIDKLNYLVDKKKEPHKLGGLLNALQLTSKFQSFEKMGKQNGFLFYVPAWNTSKIDPVTGFVNLFDTRYASVEKSKAFFTKFQSICYNEAKDYFELVFDYNDFTEKAKETRSEWTLCTYGERIVSFRNAEKNHQWDSKTIHLTTEFKNLFGELHGNDVKEYILEQNSVEFFKSLIYLLKITLQMRNSITGTDIDYLVSPVADEAGNFYDSRKADTSLPKDADANGAYNIARKGLMLMHRIQNAEDLKKVNLAISNRDWLRNAQGLDK

SEQ ID NO: 19 - protein, 1227 aa, unidentified
MKEQFINCYPLSKTLQFSLIPVGKTDDNFNKKLLLERDKQRAENYEKVKGYIDRFHKEYIESVLFNARVEKIDEYADLYWKSNKDDSDAKAMESLENDMRKQISKQLKSNARYKRLFGKELICEDLPSFLTDKKERETVECFRSFTTYFKGFNTNRENMYSSDEKSTAIAYRCINDNLPRFLDNVKSFQKVFDNLSDETITKLNTDLYNTFGRNIEDIFSVDYFEFVLAQSGIDIYNSMIGGYTCSDGTKIQGLNECINLYNQQAAKNEKSKRLPLMKQLYKQILSEKDSVSFIPEKFNSDNEVLLSIEDYYSSHIGDLDLLTELLQSLNTYNANGIFVKSGAAVSDISNGAFNSWNVLRLAWNEKYEALHPVTSKTKLDNYIEKRDKIYKAIKSFSLFELQSLGNENGNEITDWYISSSKECNSKIKEAYLQARELLKSDYEKSYNKRLSKNGKATELVKNLLDAIKNFQHLVKSLNGTGKEENKDELFYGKLTSYYDSIADIDRLYDKVRNYITQKPYSKDKIKLNFDNPQLLGGWDKNKESDYRTVLLRKDDFYYLAVMDKSHSKAFVDAPNITSEDEDYYEKMEYKLLPGPNKMLPKVFFAAKNIDTFQPSDRILDIRKRESFKKGATFNKSECHEFIDYFKNSIEKHDDWSQFGFEFTPTENYNDISEFYREISDQGYSVSFNKISKSYVDELVDNGYIYLFQIYNKDFSKYSKGTPNLHTLYFKMLFDERNLSNVVYKLNGEAEMFYREASINDKEKITHQANQPIKNKNPDNEKKESTFEYDIIKDKRFTKRQFSLHVPITINFKAHGQEFLNYDVRKAVKYKDDNYVIGIDRGERNLIYISVIDSNGKIVEQISLNEIISDNGHRVDYQKLLDTKEKERDKARKNWTSVENIKELKEGYISQVVHKICELVVKYDAVIAMEDLNFGFKRGRFPVEKQVYQKFENMLISKLNLLIDKKADPTEDGGLLRAYQLTNKFDGVNKGKQNGIIFYVPAWDTSKIDPVTGFVNLLKPKYTSVSEAKKLFETIDDIKYNANTDMFEFCIDYGKFPRCNSDYKNTWTVCTNSSRILTCRNKEKNNMWDNKQIVLTDKFKSLFNEFGIDYKGNLKTSILSISNADFYRRLIKLLSLTLQMRNSITGSTLPEDDYLISPVANDRGEFYDSRNYKGTNAALPCDADANGAYNIARKALWAINVLKSTPDDMLNKANLSITNAEWLEYTQK

SEQ ID NO: 20 - protein, 1236 aa, unidentified
MKQLSELTGLYSLSKTLRFELKPIGKTLEHIESKGLIAQDEQRAEEYKKVKDIIDRYHKKFITMCLSDFKFEQNSLETYVALAEDTNRDEKAFDEIKKTLRKQIVETFKKGGSYSDLFKKELILKHLPEFVSDEEEKKMIANFSKFTTYFNGFNENRKNMYSDEDKSTAIAYRLIHENLPMFVNNMKSFSRIADSDVHQHFTEIESSFSEYLGVEHLEEMFQLDYFSETLTQEQIAVYNHIIGGRALEDGTKLKGINEYVNLYNQQHKDSRLPLLKPLYKMILSDRVALPWFPSEEFASDEEMIRAINETYNTLKEVLSIDGDSSLRSLLLNISSYDIKHIYIANDLGLTDISQQMFGQYDVYTSAIKQELREKNAPTAKERRNPELYDERINKLFKSARSFSIEHLNSLVDSEHTIQNYYKHLGAYDRNGEQRVNLFTQIEMAYVAAKEILSGKHGNINQSDAETAIIKGLLDAFKSMQRFIKPLLGNGEEADKDNEFDAKLREAWNTLDIVTPLYNKVRNWLTRKPYSTEKIKLNFENAQLLGGWDVNKEPDCTSILLRKNGLYYLAIMNKKYNHAFDIDNLPNDGKCYEKIDYKLLPGANKMLPKVFFSKSRIKEFAPSEIIVNAYKKGSHKKGANFSISDCRRLIDFFKASISKHEDWKNFPFSFSSTETYEDISGFYREVEQQGYMLGFRNVSESFIKQLVDEGKLYLFQIWNKDFSDFSKGTPNMHTLYWKMLFDEHNLANVVYKLNGQAEVFYRKSSLDVANTTIHKAHQPIMNKNQENKKRQSTFDYDIIKNRRYTVDKFQFHVPISINFKATGRDNVNDNVLDIIRNGGIKHIIGIDRGERHLLYISLIDLKGNIVKQMTLNDIVNEYNGNTYATNYKNLLAEREGNRTEARKNWKKIENIKDIKKGYLSQVVHIISKMMVEYNAIVVLEDLNMGFMRGRQKIERSVYEQFEKMLIDKLNYYVDKQKNSDEAGGLLHALQLTSKFESFKKLGKQSGCLFYIPAWNTSKIDPVTGFVNLFDTRYTNADQARKFFSQFDSIRYNADKDWFEFAFDYNKFTAKAKDTRTKWTLCTYGTRIRTFRNPARLNQWDNEEVVLTDEFKKAFANADVNIHGNLKEAICSLQDKKYLEPLMHLMKLLLQMRNSITNSEVDYLLSPVADENGTFYDSRVCSASLPKDADANGAYNIARKGLWAIHQIQETPAGDRPNLAIKNKDWLNFAQQKPYMNE

SEQ ID NO: 21 - protein, 1238 aa, unidentified
MNTYEELTGLYSLIKTINFALNPIGETLRHIEEKGFIAQDEQRAEDYKKVKDIIDRYHKYFITMCLDGFKFSTDKLMKYVALAENAKRDEDAFDEVKTQLRKEIVAAFEKGETYKDLFKKELILKHLPNFVTDENEKRLVASFNKFTTYFKKFHKCRKNMYSDEAKSTAIAHRLIHENLPMFYDNMKSFKKIAACEDCIDLAEFEAAFSEEFKAFKVEHLADIFQLEYFNNTLTQRKIDFYKLVVGGKVLEDGTKLKGVNEYVNLYNQQHKDARLPLLKPLYKMILSDKVALSWLPEEFKSDEEMVEAIQEMHKNLKGALTDNAENNLRDLLLNMGKYDLAHIYIANDLGLRDISQNIFGQYDVYTSALKQALRNEYAPTTDECQNLELYDERINKLFKSRTSFSIAELNSLADCDHTIENHFKQLGAAERNGKQEIDLFSQIETAYAKASDILSGKHGNIGQSEAETALVKNLLDAYKSLQHFIKPLLGHGDEAEKDNEFDAKLRAAWDALDIINPLYNKVRNWLTRKPYSTEKIKLNFENAQLLDGWDKNKETVNTSVLLRKDGMYYLAIMNKKANDAFACDELPSDGDCYEKIDYKQISNARMDLPRVLLPPKNIDKYAPSEEIKDIYSKRTYRKGKNFNLEHCRRLIDFFKRSITKHKDWKNFQFIFSPTEKYECISSFYNEVEQQAYMLDFRKVSVAYINQLVESGNLYLFQIWNKDFSKFSKGTPNMHTLYWKQLFDERNLADVVYKLNWQAEVFYRKRSLKLEETTVHHAHQPIPNKNKQNEKRQSQFNYDIVKNRRFTVDKFQFHVSITLNFKATGGGDINDKVLEIIRNNGIKHVIGIDRGERHLLYLSLIDLKGNIIKQMTLNDIVNEYRGHTYTTNYKDLLAEREDNRTEARRNWKRIDNIKEIKQGYLAQVVHIISKMMVEYKAIVILEDLNMGFMRRWQKIERSVYEQFEKSLIEKLNYYVDKQKDENEVGGLLHAFQLTSKFKSFKELGKQSGCLFYVPAWNTSKIDPVTGFVNLFDTKNVKGEKVRAFFSNFDAIRYNAERDWFEFAFNYSNFTNKARDTREEWTLCTHGTSIRTFRNPSKINHRDSEEVVLTDEFKKVFEKAGIDICGNLKNAICALKEKAHLEKLMQLMKLLLQMRNSKPNTEVDYMISPVADEQGNFYDSRCGNSALPDNADANGAYNIARKGLWVIKQIRETPAGKRPDWAIKNKEWLKFAQQKPYLDD

SEQ ID NO: 22 - protein, 1245 aa, unidentified
MTLIIKYKESKNKREVYMSNLYSNLHNLYQVQKTLRFELKPQGKTKENMEKVGILEADEHRAEIYSKVKKYCDEYHKLFIDKSLSNIELNGIDRYYELYSINNRDDKQKEELDQLEASLRKQISDAFKKSAEYKGLFQKDIITSYLVTMYKENQEKMQDIGEFNRFTTYFTGYNKNRENMYSEEDKSTAISYRLINENLPTFIDNIKIYKKIVSLMPEDIEKIYKDLEEYIQVDSIDEIFNISYYNDVLTQRGIECYNILISGRTKNDGDKIKGLNEYINEFNQTHNEKIPKLQELYKQILSDAESASFKIDVIKNDKELMNLIEVYYANILPILNKIEDLFTRISNYNLELILVNNDGTLSTLSNMVFNEWSYIKGAISEKYDEEYSGKEKYGTEKYAQKKQEYLKKQKIYSLKFLNDCIGNNAICEYLKNYIIQNKNIETIKEDYNEVQNIKVEDDTKELIKDEKSIEKIKKFLDDVKSLQEFVKLVIPKDRTVEKDAKFYSELTPYYEKIKEIIPLYNKVRNYVTQKPYSTEKIKLNFECPTLLNGWDANKEEANLGVILLKEGKYYLGIINPYCKKIFEVDEKDSNEQNNYKKMEYKLLPGPNKMLPKVFFSNSRIEEFNPSKELQEKYNKGYHKKGKDFDINFCHELIDFYKQSLNKHEDWKKFNFKFKDTSEYNDISEFYREVEKQGYKIEYTEYSEKYINELVDRGELYLFQIYNKDFSEYSKGKENLHTLYWKAVFDPDNIMNPVYKLNGNAEVFYRKKSLEMKVTHPANQPIANKNISTIEAGRSTSTFKYDLIKDKRYTMDKFQFHVPITMNFKSERLLNINQIVNKYLKYNDDIHVIGIDRGERNLLYVCVIDKNEKIVYQKSLNEIVSEYNNNRYTTDYHGLLDRKEKEREIAREDWKNIENIKELKEGYMSQVIHILVELMKKYNAIIVIEDLNKGFKNSRIKVEKQVYQKFEKMFIDKLNYLVFKDEDKMDEGGVLNAYQLTNKFESFTKLGKQSGILYYIPAWCTSKIDPTTGFINRFYLKYENFDKSKEFVNRIDDIRYNEKENLFEFDIDYSKFTDRLNDTKNKWTLCSYGERILTQKNANGEWFDRRIQLSIEFKKLFEKYGINLNNIKDSILKLDKDNLEFYKGNGESLGFIQLFKLMVQMRNSLTGKEEDNLISPVKNQHGKFFNTSEKIEGLPIDADANGAYNIARKGFMLVEQMKNVEDEKLNKIKYNITEKEWLNYVQNRGM

SEQ ID NO: 23 - protein, 1249 aa, unidentified
MDNLLTFSSFTNLYSLSKTLRFKLVPIGKTYDNIVASGILDEDRHRAESYKKVKVIIDDYHKAYIESVLDGYRLPVKSQGKLDSLEEYFYYYHLGQKSDKERDTFKKIQAALRKCIAKQLKNSERFSRIFKKELIQQDLIDFVSGSPEADNEISLISEFKNFTVYFTGFHENRANMYSEEEKSTAIAYRLINENLPKFVDNIDVFRKIANTEIAENFKSLYKTFEEYLQVEKLEDLFSLDYFSFVLTQKHIDVYNAIIGGKTTEQGEKVQGLNELINIYNQHHKTEKLPKLKFLFKQILSDKEALSWLPDEIESDADLLNSIKDFYDNVVKNLVDNATLKLLMESIANYDLSGIFIRNDQQLTEISQRVAGGWGVISNAVKKYLEATNVQKKKETFENYEERIDKLFKSYPSFSIAFLNQTLAESGLPNVRIEDYFRQSGAVNSDTVQKENLFARITNAYTMVESLLKCQLPSGYNLAQDSQSVALIKQLLDSIKELQLFIKPLLGAGDEADKNETFYGDFCEYWNELDILTPMYNKVRNYLTKKPYSEDKIKLNFKNPTLLDGWDLNKESDNLSVILRRDGSYFLAIMDRNFRKAFVNTPSLDLGEPYYEKMEYKLLPGPNKMLPKVFFSKSRVAEFNPSKELLDKYERGTHKKGDNFNLSDCHDLIDFFKASIQKHDDWKGFGFKFSETSSYSDLSGFYREVEMQGYKLSFAKVSADYVEKLVDEDKLYLFQIYNKDFSSYSKGTPNMHTLYWKMLFDERNLADVVYKLNGQAEVFFRKASLQQGKPTHPANVPLKNKNSLNPKSESVFGYDLIKDKRYTVDQFQLHVPITLNFKTAGQSNINQLVNDYIRTADDLHVIGIDRGERHLLYLVVIDSKGKICEQFSLNEIVNECKGISYKTNYHDLLNQKEQERLNARRSWQSIANIKELKEGYLSQVVHKIAELMVKYKAIVVLEDLNTGFKRGRQKVEKQVYQKFEKMLIDKLNFLAFKNENPELPGGILHAYQLTNKFDSFVNLGKQSGFLFYIPAWNTSKIDPMTGFVNLFDVKYESVEKAKAFFSRFDFIRYQSDKDWFEFEFDYNHFTQKAEGTKTHWTLCTYGTRIRTFRNVAKNSQWDNEEINLTDAFKSLFEKFNIDRHSNLKEELSEISEKEFFVELIHLFKLTLQMRNSVTGTDIDYLISPVMAKNGEFYDSRKADDSLPCNADANGAYNIARKGLMLQERIRMSDDQKKINFKITNKEWLDFAQNL

SEQ ID NO: 24 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 25 - DNA, 21 bp, synthetic construct
  source 1..21: mol_type = other DNA
aatttctactcttgtagatat

SEQ ID NO: 26 - protein, 1300 aa, synthetic construct
MNFQPFFQKFVHLYPISKTLRFELIPQGATQKFISEKQVLLQDEIRARKYPEMKQAIDGYHKDFIQRALSNIDSQVFEQALNTFEDLFLRSQAERATDAYKKDFETAQTKLRELIVHSFEKGEFKQEYKSLFDKNLITNLLKPWVEQQNQIGDSNYTYHEDFNKFTTYFLGFHRNRKNIYSKDPHKTALAYRLIHENLPKFLENNKILLKIQNDHPSLWEQLQTLNQTMPQLFDGWDFSQLMQVSFFSNTLTQTGIDQYNTIIGGISEGENRQKIQGINELINLYNQKQDKKNRVAKLKQLYKQILSDRSTLSFLPEKFVDDTELYHAINMFYLEHLHHQSMINGHSYTLLERVQLLINELANYDLSKVYLAPNQLSTVSHQMFGDFGYIGRALNYYYMQVIQPDYEQLLASAKTTKKIEATEKLKTIFLDTPQSLVVIQAAIDEYIQLQPSTKPHTQLTDFIISLLKQYETVADDQSIKVINVFSDIEGKYSCIKGLVNTKSESKREVLQDEKLATDIKAFMDAVNNVIKLLKPFSLNEKLVASVEKDARFYSDFEEIYQSLLIFVPLYNKVRNYITQKPYSTEKFKLNFNKPTLLSGWDANKEADNLSILLRKNGNYYLAIMDTAKGANKAFEPKTLNQLKVDDTTDCYEKMVYKLLSGPSKMFPKAFKAKNNEGNYYPTPELLTSYNNNEHLKNDKNFTLASLHAYIDWCKEYINRNPSWHQFNFKFSPTQSFQDISQFYSEVSSQSYKVHFQTIPSDYIDQLVAEGKLYLFQIYNKDFSPNAKGKENLHTLYFKALFSDENLKQPVFKLSGEAEMFYRPASLQLANTTIHKAGEPMAAKNPLTPNATRTLAYDIIKDRRFTTDKYLLHVPISLNFHAQESMSIKKHNDLVRQMIKHNHQDLHVIGIDRGEKHLLYVSVIDLKGNIVYQESLNSIKSEAQNFETPYHQLLQHREEGRAQARTAWGKIENIKELKDGYLSQVVHRIQQLILKYNAIVMLEDLNFGFKRGRFKIEKQIYQKFEKALIHKLNYVVDKSTQADELGGVRKAYQLTAPFESFEKLGKQSGVLFYVPAWNTSKIDPVTGFVDLLKPKYENLDKAQAFFNAFDSIHYNAQKNYFEFKVNLKQFAGLKAQAAQAEWTICSYGDERHVYQKKNAQQGETVIVNVTEELKVLFAKNNIEVAQSVELKETICTQTQVDFFKRLMWLLQVLLALRYSSSKDKLDYILSPVANAQGEFFDSRHASVQLPQDSDANGAYHIALKGLWVIEQLKAADNLDKVKLAISNDDWLHFAQQKPYLA

SEQ ID NO: 27 - protein, 1300 aa, synthetic construct
MNFQPFFQKFVHLYPISKTLRFELIPQGATQKFISEKQVLLQDEIRARKYPEMKQAIDGYHKDFIQRALSNIDSQVFEQALNTFEDLFLRSQAERATDAYKKDFETAQTKLRELIVHSFEKGEFKQEYKSLFDKNLITNLLKPWVEQQNQIGDSNYTYHEDFNKFTTYFLGFHENRKNIYSKDPHKTALAYRLIHENLPKFLENNKILLKIQNDHPSLWEQLQTLNQTMPQLFDGWDFSQLMQVSFFSNTLTQTGIDQYNTIIGGISEGENRQKIQGINELINLYNQKQDKKNRVAKLKQLYKQILSDRSTLSFLPEKFVDDTELYHAINMFYLEHLHHQSMINGHSYTLLERVQLLINELANYDLSKVYLAPNQLSTVSHQMFGDFGYIGRALNYYYMQVIQPDYEQLLASAKTTKKIEATEKLKTIFLDTPQSLVVIQAAIDEYIQLQPSTKPHTQLTDFIISLLKQYETVADDQSIKVINVFSDIEGKYSCIKGLVNTKSESKREVLQDEKLATDIKAFMDAVNNVIKLLKPFSLNEKLVASVEKDARFYSDFEEIYQSLLIFVPLYNKVRNYITQKPYSTEKFKLNFNKPTLLRGWDANKEADNLSILLRKNGNYYLAIMDTAKGANKAFEPKTLNQLKVDDTTDCYEKMVYKLLSGPSKMFPKAFKAKNNEGNYYPTPELLTSYNNNEHLKNDKNFTLASLHAYIDWCKEYINRNPSWHQFNFKFSPTQSFQDISQFYSEVSSQSYKVHFQTIPSDYIDQLVAEGKLYLFQIYNKDFSPNAKGKENLHTLYFKALFSDENLKQPVFKLSGEAEMFYRPASLQLANTTIHKAGEPMAAKNPLTPNATRTLAYDIIKDRRFTTDKYLLHVPISLNFHAQESMSIKKHNDLVRQMIKHNHQDLHVIGIDRGEKHLLYVSVIDLKGNIVYQESLNSIKSEAQNFETPYHQLLQHREEGRAQARTAWGKIENIKELKDGYLSQVVHRIQQLILKYNAIVMLEDLNFGFKRGRFKIEKQIYQKFEKALIHKLNYVVDKSTQADELGGVRKAYQLTAPFESFEKLGKQSGVLFYVPAWNTSKIDPVTGFVDLLKPKYENLDKAQAFFNAFDSIHYNAQKNYFEFKVNLKQFAGLKAQAAQAEWTICSYGDERHVYQKKNAQQGETVIVNVTEELKVLFAKNNIEVAQSVELKETICTQTQVDFFKRLMWLLQVLLALRYSSSKDKLDYILSPVANAQGEFFDSRHASVQLPQDSDANGAYHIALKGLWVIEQLKAADNLDKVKLAISNDDWLHFAQQKPYLA

SEQ ID NO: 28 - protein, 1300 aa, synthetic construct
MNFQPFFQKFVHLYPISKTLRFELIPQGATQKFISEKQVLLQDEIRARKYPEMKQAIDGYHKDFIQRALSNIDSQVFEQALNTFEDLFLRSQAERATDAYKKDFETAQTKLRELIVHSFEKGEFKQEYKSLFDKNLITNLLKPWVEQQNQIGDSNYTYHEDFNKFTTYFLGFHENRKNIYSKDPHKTALAYRLIHENLPKFLENNKILLKIQNDHPSLWEQLQTLNQTMPQLFDGWDFSQLMQVSFFSNTLTQTGIDQYNTIIGGISEGENRQKIQGINELINLYNQKQDKKNRVAKLKQLYKQILSDRSTLSFLPEKFVDDTELYHAINMFYLEHLHHQSMINGHSYTLLERVQLLINELANYDLSKVYLAPNQLSTVSHQMFGDFGYIGRALNYYYMQVIQPDYEQLLASAKTTKKIEATEKLKTIFLDTPQSLVVIQAAIDEYIQLQPSTKPHTQLTDFIISLLKQYETVADDQSIKVINVFSDIEGKYSCIKGLVNTKSESKREVLQDEKLATDIKAFMDAVNNVIKLLKPFSLNEKLVASVEKDARFYSDFEEIYQSLLIFVPLYNKVRNYITQKPYSTEKFKLNFNKPTLLSGWDANREADNLSILLRKNGNYYLAIMDTAKGANKAFEPKTLNQLKVDDTTDCYEKMVYKLLSGPSKMFPKAFKAKNNEGNYYPTPELLTSYNNNEHLKNDKNFTLASLHAYIDWCKEYINRNPSWHQFNFKFSPTQSFQDISQFYSEVSSQSYKVHFQTIPSDYIDQLVAEGKLYLFQIYNKDFSPNAKGKENLHTLYFKALFSDENLKQPVFKLSGEAEMFYRPASLQLANTTIHKAGEPMAAKNPLTPNATRTLAYDIIKDRRFTTDKYLLHVPISLNFHAQESMSIKKHNDLVRQMIKHNHQDLHVIGIDRGEKHLLYVSVIDLKGNIVYQESLNSIKSEAQNFETPYHQLLQHREEGRAQARTAWGKIENIKELKDGYLSQVVHRIQQLILKYNAIVMLEDLNFGFKRGRFKIEKQIYQKFEKALIHKLNYVVDKSTQADELGGVRKAYQLTAPFESFEKLGKQSGVLFYVPAWNTSKIDPVTGFVDLLKPKYENLDKAQAFFNAFDSIHYNAQKNYFEFKVNLKQFAGLKAQAAQAEWTICSYGDERHVYQKKNAQQGETVIVNVTEELKVLFAKNNIEVAQSVELKETICTQTQVDFFKRLMWLLQVLLALRYSSSKDKLDYILSPVANAQGEFFDSRHASVQLPQDSDANGAYHIALKGLWVIEQLKAADNLDKVKLAISNDDWLHFAQQKPYLA

SEQ ID NO: 29 - protein, 1300 aa, synthetic construct
MNFQPFFQKFVHLYPISKTLRFELIPQGATQKFISEKQVLLQDEIRARKYPEMKQAIDGYHKDFIQRALSNIDSQVFEQALNTFEDLFLRSQAERATDAYKKDFETAQTKLRELIVHSFEKGEFKQEYKSLFDKNLITNLLKPWVEQQNQIGDSNYTYHEDFNKFTTYFLGFHRNRKNIYSKDPHKTALAYRLIHENLPKFLENNKILLKIQNDHPSLWEQLQTLNQTMPQLFDGWDFSQLMQVSFFSNTLTQTGIDQYNTIIGGISEGENRQKIQGINELINLYNQKQDKKNRVAKLKQLYKQILSDRSTLSFLPEKFVDDTELYHAINMFYLEHLHHQSMINGHSYTLLERVQLLINELANYDLSKVYLAPNQLSTVSHQMFGDFGYIGRALNYYYMQVIQPDYEQLLASAKTTKKIEATEKLKTIFLDTPQSLVVIQAAIDEYIQLQPSTKPHTQLTDFIISLLKQYETVADDQSIKVINVFSDIEGKYSCIKGLVNTKSESKREVLQDEKLATDIKAFMDAVNNVIKLLKPFSLNEKLVASVEKDARFYSDFEEIYQSLLIFVPLYNKVRNYITQKPYSTEKFKLNFNKPTLLRGWDANREADNLSILLRKNGNYYLAIMDTAKGANKAFEPKTLNQLKVDDTTDCYEKMVYKLLSGPSKMFPKAFKAKNNEGNYYPTPELLTSYNNNEHLKNDKNFTLASLHAYIDWCKEYINRNPSWHQFNFKFSPTQSFQDISQFYSEVSSQSYKVHFQTIPSDYIDQLVAEGKLYLFQIYNKDFSPNAKGKENLHTLYFKALFSDENLKQPVFKLSGEAEMFYRPASLQLANTTIHKAGEPMAAKNPLTPNATRTLAYDIIKDRRFTTDKYLLHVPISLNFHAQESMSIKKHNDLVRQMIKHNHQDLHVIGIDRGEKHLLYVSVIDLKGNIVYQESLNSIKSEAQNFETPYHQLLQHREEGRAQARTAWGKIENIKELKDGYLSQVVHRIQQLILKYNAIVMLEDLNFGFKRGRFKIEKQIYQKFEKALIHKLNYVVDKSTQADELGGVRKAYQLTAPFESFEKLGKQSGVLFYVPAWNTSKIDPVTGFVDLLKPKYENLDKAQAFFNAFDSIHYNAQKNYFEFKVNLKQFAGLKAQAAQAEWTICSYGDERHVYQKKNAQQGETVIVNVTEELKVLFAKNNIEVAQSVELKETICTQTQVDFFKRLMWLLQVLLALRYSSSKDKLDYILSPVANAQGEFFDSRHASVQLPQDSDANGAYHIALKGLWVIEQLKAADNLDKVKLAISNDDWLHFAQQKPYLA

SEQ ID NO: 30 - protein, 9 aa, synthetic construct
PAAKRVKLD

SEQ ID NO: 31 - protein, 16 aa, synthetic construct
KRPAATKKAGQAKKKK

SEQ ID NO: 32 - protein, 1300 aa, unidentified
MNFQPFFQKFVHLYPISKTLRFELIPQGATQKFISEKQVLLQDEIRARKYPEMKQAIDGYHKDFIQRALSNIDSQVFEQALNTFEDLFLRSQAERATDAYKKDFETAQTKLRELIVHSFEKGEFKQEYKSLFDKNLITNLLKPWVEQQNQIGDSNYTYHEDFNKFTTYFLGFHRNRKNIYSKDPHKTALAYRLIHENLPKFLENNKILLKIQNDHPSLWEQLQTLNQTMPQLFDGWDFSQLMQVSFFSNTLTQTGIDQYNTIIGGISEGENRQKIQGINELINLYNQKQDKKNRVAKLKQLYKQILSDRSTLSFLPEKFVDDTELYHAINMFYLEHLHHQSMINGHSYTLLERVQLLINELANYDLSKVYLAPNQLSTVSHQMFGDFGYIGRALNYYYMQVIQPDYEQLLASAKTTKKIEATEKLKTIFLDTPQSLVVIQAAIDEYIQLQPSTKPHTQLTDFIISLLKQYETVADDQSIKVINVFSDIEGKYSCIKGLVNTKSESKREVLQDEKLATDIKAFMDAVNNVIKLLKPFSLNEKLVASVEKDARFYSDFEEIYQSLLIFVPLYNKVRNYITQKPYSTEKFKLNFNKPTLLSGWDANREADNLSILLRKNGNYYLAIMDTAKGANKAFEPKTLNQLKVDDTTDCYEKMVYKLLSGPSKMFPKAFKAKNNEGNYYPTPELLTSYNNNEHLKNDKNFTLASLHAYIDWCKEYINRNPSWHQFNFKFSPTQSFQDISQFYSEVSSQSYKVHFQTIPSDYIDQLVAEGKLYLFQIYNKDFSPNAKGKENLHTLYFKALFSDENLKQPVFKLSGEAEMFYRPASLQLANTTIHKAGEPMAAKNPLTPNATRTLAYDIIKDRRFTTDKYLLHVPISLNFHAQESMSIKKHNDLVRQMIKHNHQDLHVIGIDRGEKHLLYVSVIDLKGNIVYQESLNSIKSEAQNFETPYHQLLQHREEGRAQARTAWGKIENIKELKDGYLSQVVHRIQQLILKYNAIVMLEDLNFGFKRGRFKIEKQIYQKFEKALIHKLNYVVDKSTQADELGGVRKAYQLTAPFESFEKLGKQSGVLFYVPAWNTSKIDPVTGFVDLLKPKYENLDKAQAFFNAFDSIHYNAQKNYFEFKVNLKQFAGLKAQAAQAEWTICSYGDERHVYQKKNAQQGETVIVNVTEELKVLFAKNNIEVAQSVELKETICTQTQVDFFKRLMWLLQVLLALRYSSSKDKLDYILSPVANAQGEFFDSRHASVQLPQDSDANGAYHIALKGLWVIEQLKAADNLDKVKLAISNDDWLHFAQQKPYLA

SEQ ID NO: 33 - protein, 1300 aa, unidentified
MNFQPFFQKFVHLYPISKTLRFELIPQGATQKFISEKQVLLQDEIRARKYPEMKQAIDGYHKDFIQRALSNIDSQVFEQALNTFEDLFLRSQAERATDAYKKDFETAQTKLRELIVHSFEKGEFKQEYKSLFDKNLITNLLKPWVEQQNQIGDSNYTYHEDFNKFTTYFLGFHRNRKNIYSKDPHKTALAYRLIHENLPKFLENNKILLKIQNDHPSLWEQLQTLNQTMPQLFDGWDFSQLMQVSFFSNTLTQTGIDQYNTIIGGISEGENRQKIQGINELIALYNQKQDKKNRVAKLKQLYKQILSDRSTLSFLPEKFVDDTELYHAINMFYLEHLHHQSMINGHSYTLLERVQLLINELANYDLSKVYLAPNQLSTVSHQMFGDFGYIGRALNYYYMQVIQPDYEQLLASAKTTKKIEATEKLKTIFLDTPQSLVVIQAAIDEYIQLQPSTKPHTQLTDFIISLLKQYETVADDQSIKVINVFSDIEGKYSCIKGLVNTKSESKREVLQDEKLATDIKAFMDAVNNVIKLLKPFSLNEKLVASVEKDARFYSDFEEIYQSLLIFVPLYNKVRNYITQKPYSTEKFKLNFNKPTLLSGWDANREADNLSILLRKNGNYYLAIMDTAKGANKAFEPKTLNQLKVDDTTDCYEKMVYKLLSGPSKMFPKAFKAKNNEGNYYPTPELLTSYNNNEHLKNDKNFTLASLHAYIDWCKEYINRNPSWHQFNFKFSPTQSFQDISQFYSEVSSQSYKVHFQTIPSDYIDQLVAEGKLYLFQIYNKDFSPNAKGKENLHTLYFKALFSDENLKQPVFKLSGEAEMFYRPASLQLANTTIHKAGEPMAAKNPLTPNATRTLAYDIIKDRRFTTDKYLLHVPISLNFHAQESMSIKKHNDLVRQMIKHNHQDLHVIGIDRGEKHLLYVSVIDLKGNIVYQESLNSIKSEAQNFETPYHQLLQHREEGRAQARTAWGKIENIKELKDGYLSQVVHRIQQLILKYNAIVMLEDLNFGFKRGRFKIEKQIYQKFEKALIHKLNYVVDKSTQADELGGVRKAYQLTAPFESFEKLGKQSGVLFYVPAWNTSKIDPVTGFVDLLKPKYENLDKAQAFFNAFDSIHYNAQKNYFEFKVNLKQFAGLKAQAAQAEWTICSYGDERHVYQKKNAQQGETVIVNVTEELKVLFAKNNIEVAQSVELKETICTQTQVDFFKRLMWLLQVLLALRYSSSKDKLDYILSPVANAQGEFFDSRHASVQLPQDSDANGAYHIALKGLWVIEQLKAADNLDKVKLAISNDDWLHFAQQKPYLA

SEQ ID NO: 34 - protein, 1379 aa, unidentified
MNFQPFFQKFVHLYPISKTLRFELIPQGATQKFISEKQVLLQDEIRARKYPEMKQAIDGYHKDFIQRALSNIDSQVFEQALNTFEDLFLRSQAERATDAYKKDFETAQTKLRELIVHSFEKGEFKQEYKSLFDKNLITNLLKPWVEQQNQIGDSNYTYHEDFNKFTTYFLGFHRNRKNIYSKDPHKTALAYRLIHENLPKFLENNKILLKIQNDHPSLWEQLQTLNQTMPQLFDGWDFSQLMQVSFFSNTLTQTGIDQYNTIIGGISEGENRQKIQGINELINLYNQKQDKKNRVAKLKQLYKQILSDRSTLSFLPEKFVDDTELYHAINMFYLEHLHHQSMINGHSYTLLERVQLLINELANYDLSKVYLAPNQLSTVSHQMFGDFGYIGRALNYYYMQVIQPDYEQLLASAKTTKKIEATEKLKTIFLDTPQSLVVIQAAIDEYIQLQPSTKPHTQLTDFIISLLKQYETVADDQSIKVINVFSDIEGKYSCIKGLVNTKSESKREVLQDEKLATDIKAFMDAVNNVIKLLKPFSLNEKLVASVEKDARFYSDFEEIYQSLLIFVPLYNKVRNYITQKPYSTEKFKLNFNKPTLLRGWDANREADNLSILLRKNGNYYLAIMDTAKGANKAFEPKTLNQLKVDDTTDCYEKMVYKLLSGPSKMFPKAFKAKNNEGNYYPTPELLTSYNNNEHLKNDKNFTLASLHAYIDWCKEYINRNPSWHQFNFKFSPTQSFQDISQFYSEVSSQSYKVHFQTIPSDYIDQLVAEGKLYLFQIYNKDFSPNAKGKENLHTLYFKALFSDENLKQPVFKLSGEAEMFYRPASLQLANTTIHKAGEPMAAKNPLTPNATRTLAYDIIKDRRFTTDKYLLHVPISLNFHAQESMSIKKHNDLVRQMIKHNHQDLHVIGIDRGEKHLLYVSVIDLKGNIVYQESLNSIKSEAQNFETPYHQLLQHREEGRAQARTAWGKIENIKELKDGYLSQVVHRIQQLILKYNAIVMLEDLNFGFKRGRFKIEKQIYQKFEKALIHKLNYVVDKSTQADELGGVRKAYQLTAPFESFEKLGKQSGVLFYVPAWNTSKIDPVTGFVDLLKPKYENLDKAQAFFNAFDSIHYNAQKNYFEFKVNLKQFAGLKAQAAQAEWTICSYGDERHVYQKKNAQQGETVIVNVTEELKVLFAKNNIEVAQSVELKETICTQTQVDFFKRLMWLLQVLLALRYSSSKDKLDYILSPVANAQGEFFDSRHASVQLPQDSDANGAYHIALKGLWVIEQLKAADNLDKVKLAISNDDWLHFAQQKPYLAGRSSDDEATADSQHAAPPKKKRKVGGSGGSGGSGGSGGSGGSGGSGGSLEGSYPYDVPDYAYPYDVPDYAYPYDVPDYA

SEQ ID NO: 35 - protein, 1300 aa, unidentified
MNFQPFFQKFVHLYPISKTLRFELIPQGATQKFISEKQVLLQDEIRARKYPEMKQAIDGYHKDFIQRALSNIDSQVFEQALNTFEDLFLRSQAERATDAYKKDFETAQTKLRELIVHSFEKGEFKQEYKSLFDKNLITNLLKPWVEQQNQIGDSNYTYHEDFNKFTTYFLGFHENRKNIYSKDPHKTALAYRLIHENLPKFLENNKILLKIQNDHPSLWEQLQTLNQTMPQLFDGWDFSQLMQVSFFSNTLTQTGIDQYNTIIGGISEGENRQKIQGINELINLYNQKQDKKNRVAKLKQLYKQILSDRSTLSFLPEKFVDDTELYHAINMFYLEHLHHQSMINGHSYTLLERVQLLINELANYDLSKVYLAPNQLSTVSHQMFGDFGYIGRALNYYYMQVIQPDYEQLLASAKTTKKIEATEKLKTIFLDTPQSLVVIQAAIDEYIQLQPSTKPHTQLTDFIISLLKQYETVADDQSIKVINVFSDIEGKYSCIKGLVNTKSESKREVLQDEKLATDIKAFMDAVNNVIKLLKPFSLNEKLVASVEKDARFYSDFEEIYQSLLIFVPLYNKVRNYITQKPYSTEKFKLNFNKPTLLRGWDANREADNLSILLRKNGNYYLAIMDTAKGANKAFEPKTLNQLKVDDTTDCYEKMVYKLLSGPSKMFPKAFKAKNNEGNYYPTPELLTSYNNNEHLKNDKNFTLASLHAYIDWCKEYINRNPSWHQFNFKFSPTQSFQDISQFYSEVSSQSYKVHFQTIPSDYIDQLVAEGKLYLFQIYNKDFSPNAKGKENLHTLYFKALFSDENLKQPVFKLSGEAEMFYRPASLQLANTTIHKAGEPMAAKNPLTPNATRTLAYDIIKDRRFTTDKYLLHVPISLNFHAQESMSIKKHNDLVRQMIKHNHQDLHVIGIDRGEKHLLYVSVIDLKGNIVYQESLNSIKSEAQNFETPYHQLLQHREEGRAQARTAWGKIENIKELKDGYLSQVVHRIQQLILKYNAIVMLEDLNFGFKRGRFKIEKQIYQKFEKALIHKLNYVVDKSTQADELGGVRKAYQLTAPFESFEKLGKQSGVLFYVPAWNTSKIDPVTGFVDLLKPKYENLDKAQAFFNAFDSIHYNAQKNYFEFKVNLKQFAGLKAQAAQAEWTICSYGDERHVYQKKNAQQGETVIVNVTEELKVLFAKNNIEVAQSVELKETICTQTQVDFFKRLMWLLQVLLALRYSSSKDKLDYILSPVANAQGEFFDSRHASVQLPQDSDANGAYHIALKGLWVIEQLKAADNLDKVKLAISNDDWLHFAQQKPYLA

SEQ ID NO: 36 - protein, 1300 aa, synthetic construct
MNFQPFFQKFVHLYPISKTLRFELIPQGATQKFISEKQVLLQDEIRARKYPEMKQAIDGYHKDFIQRALSNIDSQVFEQALNTFEDLFLRSQAERATDAYKKDFETAQTKLRELIVHSFEKGEFKQEYKSLFDKNLITNLLKPWVEQQNQIGDSNYTYHEDFNKFTTYFLGFHRNRKNIYSKDPHKTALAYRLIHENLPKFLENNKILLKIQNDHPSLWEQLQTLNQTMPQLFDGWDFSQLMQVSFFSNTLTQTGIDQYNTIIGGISEGENRQKIQGINELINLYNQKQDKKNRVAKLKQLYKQILSDRSTLSFLPEKFVDDTELYHAINMFYLEHLHHQSMINGHSYTLLERVQLLINELANYDLSKVYLAPNQLSTVSHQMFGDFGYIGRALNYYYMQVIQPDYEQLLASAKTTKKIEATEKLKTIFLDTPQSLVVIQAAIDEYIQLQPSTKPHTQLTDFIISLLKQYETVADDQSIKVINVFSDIEGKYSCIKGLVNTKSESKREVLQDEKLATDIKAFMDAVNNVIKLLKPFSLNEKLVASVEKDARFYSDFEEIYQSLLIFVPLYNKVRNYITQKPYSTEKFKLNFNKPTLLRGWDANKEADNLSILLRKNGNYYLAIMDTAKGANKAFEPKTLNQLKVDDTTDCYEKMVYKLLSGPSKMFPKAFKAKNNEGNYYPTPELLTSYNNNEHLKNDKNFTLASLHAYIDWCKEYINRNPSWHQFNFKFSPTQSFQDISQFYSEVSSQSYKVHFQTIPSDYIDQLVAEGKLYLFQIYNKDFSPNAKGKENLHTLYFKALFSDENLKQPVFKLSGEAEMFYRPASLQLANTTIHKAGEPMAAKNPLTPNATRTLAYDIIKDRRFTTDKYLLHVPISLNFHAQESMSIKKHNDLVRQMIKHNHQDLHVIGIDRGEKHLLYVSVIDLKGNIVYQESLNSIKSEAQNFETPYHQLLQHREEGRAQARTAWGKIENIKELKDGYLSQVVHRIQQLILKYNAIVMLEDLNFGFKRGRFKIEKQIYQKFEKALIHKLNYVVDKSTQADELGGVRKAYQLTAPFESFEKLGKQSGVLFYVPAWNTSKIDPVTGFVDLLKPKYENLDKAQAFFNAFDSIHYNAQKNYFEFKVNLKQFAGLKAQAAQAEWTICSYGDERHVYQKKNAQQGETVIVNVTEELKVLFAKNNIEVAQSVELKETICTQTQVDFFKRLMWLLQVLLALRYSSSKDKLDYILSPVANAQGEFFDSRHASVQLPQDSDANGAYHIALKGLWVIEQLKAADNLDKVKLAISNDDWLHFAQQKPYLA

SEQ ID NO: 37 - protein, 1197 aa, synthetic construct
MNIYENFTNMYQVNKTIRMGLKPICKTDENIAKFLEEDKERSEKYKIAKKIIDKENRAFIEDRLKDFSISGLDEYLELLKQKKDITKIQKKMRDEISKQLKGFPQFDSKYKFQYITDKEDTEILEYFKKFTTFFTGFNRNRENVYSKEDISTSIGHRIIHENLPKFISNFRILNKAIEALGTGKINEDFKNNEINVTVEELNKIDYFNKVLTQSGIDLYNNLIGILNQNINLYNQQQKVKKNKIGKLETLHKQILSEKDKVSFIEEFAEDNQLLKCIDEYFKEKSCLINVDLKNLLENIDTYSLNGIFIKNDKSLKNISIYLYKDWGYISNLINEEYDYKHKNKVKDDKYYEKRKKAIDKIKYFSIGYIDELLKDKNVPMVECYFKEKINLVVKEFIDYLNKFNEYKFTNELKTDEIAVEIIKNLCDSIKDVQEIVKPLIVIENDKDDSFYGEINYIWDELNKFNKIYNMVRNYLTKKDYIDEKIRLTFNNPQLLGGWSYKKEADYSSFILKDNKYYYLAIMNPNSKSKLKDLKKPENDGDTIYKMKYFQASDPAKSIPNLMVINGKTEKKNGRKDNDGENRELERLRNEYLPTEINEIRKNKAYLRSSSNFSNEALVKYIDYYKERVKEYFNEIDFKFKETCEYKQFNEFAEDVNLQAYQISFIEVSKKYIKSLIDDNKIYLFKIYNKDFSKYSKGTPNLHTLYFKMLFDKENLENPIYKLSGNAEMFFRKGNLDLDKTTIHHANQPINNKNPNNRKKQSVFKYDIIKNRRYTVDKFALHMSITTNFQVYKNKNVNETVNRALKYCDDIYAIGIDRGERNLLYACVVNSRGEIVKQVPLNFVCNTDYHQLLAKREEERMNSRKNWKIIDNIKNLKEGYLSQAIHIITDFMVEYNAVLVLEDLNFRFKEKRMKFEKSVYQKFEKMLIDKLNFLVDKKLDKNANGGLFNAYQLTEKFTSFKDMKNQNGIVFYIPAWMTSKIDPVTGFTNLFYIKYESIEKSKEFFGKFKSIKFNKVDNYFEFEFDYNDFTDRAQGTRSKWTVCSFGPRIEGFRNPEKNNKWDGREIDITEKIKKLLDDYNISLDEDIQAQIMDINTKDFFEKLIKYFKLVLQMRNSKTGTDIDYIISPVRNKQNEFFDSRKKNEKLPMDADANGAYNIARKGLMFIDIIKETEDKDLKMPKLFIKNKDWLNYVQKSDL

SEQ ID NO: 38 - protein, 1197 aa, synthetic construct
MNIYENFTNMYQVNKTIRMGLKPICKTDENIAKFLEEDKERSEKYKIAKKIIDKENRAFIEDRLKDFSISGLDEYLELLKQKKDITKIQKKMRDEISKQLKGFPQFDSKYKFQYITDKEDTEILEYFKKFTTFFTGFNSNRENVYSKEDISTSIGHRIIHENLPKFISNFRILNKAIEALGTGKINEDFKNNEINVTVEELNKIDYFNKVLTQSGIDLYNNLIGILNQNINLYNQQQKVKKNKIGKLETLHKQILSEKDKVSFIEEFAEDNQLLKCIDEYFKEKSCLINVDLKNLLENIDTYSLNGIFIKNDKSLKNISIYLYKDWGYISNLINEEYDYKHKNKVKDDKYYEKRKKAIDKIKYFSIGYIDELLKDKNVPMVECYFKEKINLVVKEFIDYLNKFNEYKFTNELKTDEIAVEIIKNLCDSIKDVQEIVKPLIVIENDKDDSFYGEINYIWDELNKFNKIYNMVRNYLTKKDYIDEKIRLTFNNPQLLRGWSYKKEADYSSFILKDNKYYYLAIMNPNSKSKLKDLKKPENDGDTIYKMKYFQASDPAKSIPNLMVINGKTEKKNGRKDNDGENRELERLRNEYLPTEINEIRKNKAYLRSSSNFSNEALVKYIDYYKERVKEYFNEIDFKFKETCEYKQFNEFAEDVNLQAYQISFIEVSKKYIKSLIDDNKIYLFKIYNKDFSKYSKGTPNLHTLYFKMLFDKENLENPIYKLSGNAEMFFRKGNLDLDKTTIHHANQPINNKNPNNRKKQSVFKYDIIKNRRYTVDKFALHMSITTNFQVYKNKNVNETVNRALKYCDDIYAIGIDRGERNLLYACVVNSRGEIVKQVPLNFVCNTDYHQLLAKREEERMNSRKNWKIIDNIKNLKEGYLSQAIHIITDFMVEYNAVLVLEDLNFRFKEKRMKFEKSVYQKFEKMLIDKLNFLVDKKLDKNANGGLFNAYQLTEKFTSFKDMKNQNGIVFYIPAWMTSKIDPVTGFTNLFYIKYESIEKSKEFFGKFKSIKFNKVDNYFEFEFDYNDFTDRAQGTRSKWTVCSFGPRIEGFRNPEKNNKWDGREIDITEKIKKLLDDYNISLDEDIQAQIMDINTKDFFEKLIKYFKLVLQMRNSKTGTDIDYIISPVRNKQNEFFDSRKKNEKLPMDADANGAYNIARKGLMFIDIIKETEDKDLKMPKLFIKNKDWLNYVQKSDL

SEQ ID NO: 39 - protein, 1197 aa, synthetic construct
MNIYENFTNMYQVNKTIRMGLKPICKTDENIAKFLEEDKERSEKYKIAKKIIDKENRAFIEDRLKDFSISGLDEYLELLKQKKDITKIQKKMRDEISKQLKGFPQFDSKYKFQYITDKEDTEILEYFKKFTTFFTGFNSNRENVYSKEDISTSIGHRIIHENLPKFISNFRILNKAIEALGTGKINEDFKNNEINVTVEELNKIDYFNKVLTQSGIDLYNNLIGILNQNINLYNQQQKVKKNKIGKLETLHKQILSEKDKVSFIEEFAEDNQLLKCIDEYFKEKSCLINVDLKNLLENIDTYSLNGIFIKNDKSLKNISIYLYKDWGYISNLINEEYDYKHKNKVKDDKYYEKRKKAIDKIKYFSIGYIDELLKDKNVPMVECYFKEKINLVVKEFIDYLNKFNEYKFTNELKTDEIAVEIIKNLCDSIKDVQEIVKPLIVIENDKDDSFYGEINYIWDELNKFNKIYNMVRNYLTKKDYIDEKIRLTFNNPQLLGGWSYKREADYSSFILKDNKYYYLAIMNPNSKSKLKDLKKPENDGDTIYKMKYFQASDPAKSIPNLMVINGKTEKKNGRKDNDGENRELERLRNEYLPTEINEIRKNKAYLRSSSNFSNEALVKYIDYYKERVKEYFNEIDFKFKETCEYKQFNEFAEDVNLQAYQISFIEVSKKYIKSLIDDNKIYLFKIYNKDFSKYSKGTPNLHTLYFKMLFDKENLENPIYKLSGNAEMFFRKGNLDLDKTTIHHANQPINNKNPNNRKKQSVFKYDIIKNRRYTVDKFALHMSITTNFQVYKNKNVNETVNRALKYCDDIYAIGIDRGERNLLYACVVNSRGEIVKQVPLNFVCNTDYHQLLAKREEERMNSRKNWKIIDNIKNLKEGYLSQAIHIITDFMVEYNAVLVLEDLNFRFKEKRMKFEKSVYQKFEKMLIDKLNFLVDKKLDKNANGGLFNAYQLTEKFTSFKDMKNQNGIVFYIPAWMTSKIDPVTGFTNLFYIKYESIEKSKEFFGKFKSIKFNKVDNYFEFEFDYNDFTDRAQGTRSKWTVCSFGPRIEGFRNPEKNNKWDGREIDITEKIKKLLDDYNISLDEDIQAQIMDINTKDFFEKLIKYFKLVLQMRNSKTGTDIDYIISPVRNKQNEFFDSRKKNEKLPMDADANGAYNIARKGLMFIDIIKETEDKDLKMPKLFIKNKDWLNYVQKSDL

SEQ ID NO: 40 - protein, 1197 aa, synthetic construct
MNIYENFTNMYQVNKTIRMGLKPICKTDENIAKFLEEDKERSEKYKIAKKIIDKENRAFIEDRLKDFSISGLDEYLELLKQKKDITKIQKKMRDEISKQLKGFPQFDSKYKFQYITDKEDTEILEYFKKFTTFFTGFNRNRENVYSKEDISTSIGHRIIHENLPKFISNFRILNKAIEALGTGKINEDFKNNEINVTVEELNKIDYFNKVLTQSGIDLYNNLIGILNQNINLYNQQQKVKKNKIGKLETLHKQILSEKDKVSFIEEFAEDNQLLKCIDEYFKEKSCLINVDLKNLLENIDTYSLNGIFIKNDKSLKNISIYLYKDWGYISNLINEEYDYKHKNKVKDDKYYEKRKKAIDKIKYFSIGYIDELLKDKNVPMVECYFKEKINLVVKEFIDYLNKFNEYKFTNELKTDEIAVEIIKNLCDSIKDVQEIVKPLIVIENDKDDSFYGEINYIWDELNKFNKIYNMVRNYLTKKDYIDEKIRLTFNNPQLLRGWSYKKEADYSSFILKDNKYYYLAIMNPNSKSKLKDLKKPENDGDTIYKMKYFQASDPAKSIPNLMVINGKTEKKNGRKDNDGENRELERLRNEYLPTEINEIRKNKAYLRSSSNFSNEALVKYIDYYKERVKEYFNEIDFKFKETCEYKQFNEFAEDVNLQAYQISFIEVSKKYIKSLIDDNKIYLFKIYNKDFSKYSKGTPNLHTLYFKMLFDKENLENPIYKLSGNAEMFFRKGNLDLDKTTIHHANQPINNKNPNNRKKQSVFKYDIIKNRRYTVDKFALHMSITTNFQVYKNKNVNETVNRALKYCDDIYAIGIDRGERNLLYACVVNSRGEIVKQVPLNFVCNTDYHQLLAKREEERMNSRKNWKIIDNIKNLKEGYLSQAIHIITDFMVEYNAVLVLEDLNFRFKEKRMKFEKSVYQKFEKMLIDKLNFLVDKKLDKNANGGLFNAYQLTEKFTSFKDMKNQNGIVFYIPAWMTSKIDPVTGFTNLFYIKYESIEKSKEFFGKFKSIKFNKVDNYFEFEFDYNDFTDRAQGTRSKWTVCSFGPRIEGFRNPEKNNKWDGREIDITEKIKKLLDDYNISLDEDIQAQIMDINTKDFFEKLIKYFKLVLQMRNSKTGTDIDYIISPVRNKQNEFFDSRKKNEKLPMDADANGAYNIARKGLMFIDIIKETEDKDLKMPKLFIKNKDWLNYVQKSDL

SEQ ID NO: 41 - protein, 1197 aa, synthetic construct
MNIYENFTNMYQVNKTIRMGLKPICKTDENIAKFLEEDKERSEKYKIAKKIIDKENRAFIEDRLKDFSISGLDEYLELLKQKKDITKIQKKMRDEISKQLKGFPQFDSKYKFQYITDKEDTEILEYFKKFTTFFTGFNRNRENVYSKEDISTSIGHRIIHENLPKFISNFRILNKAIEALGTGKINEDFKNNEINVTVEELNKIDYFNKVLTQSGIDLYNNLIGILNQNINLYNQQQKVKKNKIGKLETLHKQILSEKDKVSFIEEFAEDNQLLKCIDEYFKEKSCLINVDLKNLLENIDTYSLNGIFIKNDKSLKNISIYLYKDWGYISNLINEEYDYKHKNKVKDDKYYEKRKKAIDKIKYFSIGYIDELLKDKNVPMVECYFKEKINLVVKEFIDYLNKFNEYKFTNELKTDEIAVEIIKNLCDSIKDVQEIVKPLIVIENDKDDSFYGEINYIWDELNKFNKIYNMVRNYLTKKDYIDEKIRLTFNNPQLLGGWSYKREADYSSFILKDNKYYYLAIMNPNSKSKLKDLKKPENDGDTIYKMKYFQASDPAKSIPNLMVINGKTEKKNGRKDNDGENRELERLRNEYLPTEINEIRKNKAYLRSSSNFSNEALVKYIDYYKERVKEYFNEIDFKFKETCEYKQFNEFAEDVNLQAYQISFIEVSKKYIKSLIDDNKIYLFKIYNKDFSKYSKGTPNLHTLYFKMLFDKENLENPIYKLSGNAEMFFRKGNLDLDKTTIHHANQPINNKNPNNRKKQSVFKYDIIKNRRYTVDKFALHMSITTNFQVYKNKNVNETVNRALKYCDDIYAIGIDRGERNLLYACVVNSRGEIVKQVPLNFVCNTDYHQLLAKREEERMNSRKNWKIIDNIKNLKEGYLSQAIHIITDFMVEYNAVLVLEDLNFRFKEKRMKFEKSVYQKFEKMLIDKLNFLVDKKLDKNANGGLFNAYQLTEKFTSFKDMKNQNGIVFYIPAWMTSKIDPVTGFTNLFYIKYESIEKSKEFFGKFKSIKFNKVDNYFEFEFDYNDFTDRAQGTRSKWTVCSFGPRIEGFRNPEKNNKWDGREIDITEKIKKLLDDYNISLDEDIQAQIMDINTKDFFEKLIKYFKLVLQMRNSKTGTDIDYIISPVRNKQNEFFDSRKKNEKLPMDADANGAYNIARKGLMFIDIIKETEDKDLKMPKLFIKNKDWLNYVQKSDL

SEQ ID NO: 42 - protein, 1197 aa, synthetic construct
MNIYENFTNMYQVNKTIRMGLKPICKTDENIAKFLEEDKERSEKYKIAKKIIDKENRAFIEDRLKDFSISGLDEYLELLKQKKDITKIQKKMRDEISKQLKGFPQFDSKYKFQYITDKEDTEILEYFKKFTTFFTGFNSNRENVYSKEDISTSIGHRIIHENLPKFISNFRILNKAIEALGTGKINEDFKNNEINVTVEELNKIDYFNKVLTQSGIDLYNNLIGILNQNINLYNQQQKVKKNKIGKLETLHKQILSEKDKVSFIEEFAEDNQLLKCIDEYFKEKSCLINVDLKNLLENIDTYSLNGIFIKNDKSLKNISIYLYKDWGYISNLINEEYDYKHKNKVKDDKYYEKRKKAIDKIKYFSIGYIDELLKDKNVPMVECYFKEKINLVVKEFIDYLNKFNEYKFTNELKTDEIAVEIIKNLCDSIKDVQEIVKPLIVIENDKDDSFYGEINYIWDELNKFNKIYNMVRNYLTKKDYIDEKIRLTFNNPQLLRGWSYKREADYSSFILKDNKYYYLAIMNPNSKSKLKDLKKPENDGDTIYKMKYFQASDPAKSIPNLMVINGKTEKKNGRKDNDGENRELERLRNEYLPTEINEIRKNKAYLRSSSNFSNEALVKYIDYYKERVKEYFNEIDFKFKETCEYKQFNEFAEDVNLQAYQISFIEVSKKYIKSLIDDNKIYLFKIYNKDFSKYSKGTPNLHTLYFKMLFDKENLENPIYKLSGNAEMFFRKGNLDLDKTTIHHANQPINNKNPNNRKKQSVFKYDIIKNRRYTVDKFALHMSITTNFQVYKNKNVNETVNRALKYCDDIYAIGIDRGERNLLYACVVNSRGEIVKQVPLNFVCNTDYHQLLAKREEERMNSRKNWKIIDNIKNLKEGYLSQAIHIITDFMVEYNAVLVLEDLNFRFKEKRMKFEKSVYQKFEKMLIDKLNFLVDKKLDKNANGGLFNAYQLTEKFTSFKDMKNQNGIVFYIPAWMTSKIDPVTGFTNLFYIKYESIEKSKEFFGKFKSIKFNKVDNYFEFEFDYNDFTDRAQGTRSKWTVCSFGPRIEGFRNPEKNNKWDGREIDITEKIKKLLDDYNISLDEDIQAQIMDINTKDFFEKLIKYFKLVLQMRNSKTGTDIDYIISPVRNKQNEFFDSRKKNEKLPMDADANGAYNIARKGLMFIDIIKETEDKDLKMPKLFIKNKDWLNYVQKSDL

SEQ ID NO: 43 - protein, 1197 aa, synthetic construct
MNIYENFTNMYQVNKTIRMGLKPICKTDENIAKFLEEDKERSEKYKIAKKIIDKENRAFIEDRLKDFSISGLDEYLELLKQKKDITKIQKKMRDEISKQLKGFPQFDSKYKFQYITDKEDTEILEYFKKFTTFFTGFNRNRENVYSKEDISTSIGHRIIHENLPKFISNFRILNKAIEALGTGKINEDFKNNEINVTVEELNKIDYFNKVLTQSGIDLYNNLIGILNQNIALYNQQQKVKKNKIGKLETLHKQILSEKDKVSFIEEFAEDNQLLKCIDEYFKEKSCLINVDLKNLLENIDTYSLNGIFIKNDKSLKNISIYLYKDWGYISNLINEEYDYKHKNKVKDDKYYEKRKKAIDKIKYFSIGYIDELLKDKNVPMVECYFKEKINLVVKEFIDYLNKFNEYKFTNELKTDEIAVEIIKNLCDSIKDVQEIVKPLIVIENDKDDSFYGEINYIWDELNKFNKIYNMVRNYLTKKDYIDEKIRLTFNNPQLLRGWSYKREADYSSFILKDNKYYYLAIMNPNSKSKLKDLKKPENDGDTIYKMKYFQASDPAKSIPNLMVINGKTEKKNGRKDNDGENRELERLRNEYLPTEINEIRKNKAYLRSSSNFSNEALVKYIDYYKERVKEYFNEIDFKFKETCEYKQFNEFAEDVNLQAYQISFIEVSKKYIKSLIDDNKIYLFKIYNKDFSKYSKGTPNLHTLYFKMLFDKENLENPIYKLSGNAEMFFRKGNLDLDKTTIHHANQPINNKNPNNRKKQSVFKYDIIKNRRYTVDKFALHMSITTNFQVYKNKNVNETVNRALKYCDDIYAIGIDRGERNLLYACVVNSRGEIVKQVPLNFVCNTDYHQLLAKREEERMNSRKNWKIIDNIKNLKEGYLSQAIHIITDFMVEYNAVLVLEDLNFRFKEKRMKFEKSVYQKFEKMLIDKLNFLVDKKLDKNANGGLFNAYQLTEKFTSFKDMKNQNGIVFYIPAWMTSKIDPVTGFTNLFYIKYESIEKSKEFFGKFKSIKFNKVDNYFEFEFDYNDFTDRAQGTRSKWTVCSFGPRIEGFRNPEKNNKWDGREIDITEKIKKLLDDYNISLDEDIQAQIMDINTKDFFEKLIKYFKLVLQMRNSKTGTDIDYIISPVRNKQNEFFDSRKKNEKLPMDADANGAYNIARKGLMFIDIIKETEDKDLKMPKLFIKNKDWLNYVQKSDL

SEQ ID NO: 44 - protein, 1197 aa, synthetic construct
MNIYENFTNMYQVNKTIRMGLKPICKTDENIAKFLEEDKERSEKYKIAKKIIDKENRAFIEDRLKDFSISGLDEYLELLKQKKDITKIQKKMRDEISKQLKGFPQFDSKYKFQYITDKEDTEILEYFKKFTTFFTGFNRNRENVYSKEDISTSIGHRIIHENLPKFISNFRILNKAIEALGTGKINEDFKNNEINVTVEELNKIDYFNKVLTQSGIDLYNNLIGILNQNIALYNQQQKVKKNKIGKLETLHKQILSEKDKVSFIEEFAEDNQLLKCIDEYFKEKSCLINVDLKNLLENIDTYSLNGIFIKNDKSLKNISIYLYKDWGYISNLINEEYDYKHKNKVKDDKYYEKRKKAIDKIKYFSIGYIDELLKDKNVPMVECYFKEKINLVVKEFIDYLNKFNEYKFTNELKTDEIAVEIIKNLCDSIKDVQEIVKPLIVIENDKDDSFYGEINYIWDELNKFNKIYNMVRNYLTKKDYIDEKIRLTFNNPQLLGGWSYKREADYSSFILKDNKYYYLAIMNPNSKSKLKDLKKPENDGDTIYKMKYFQASDPAKSIPNLMVINGKTEKKNGRKDNDGENRELERLRNEYLPTEINEIRKNKAYLRSSSNFSNEALVKYIDYYKERVKEYFNEIDFKFKETCEYKQFNEFAEDVNLQAYQISFIEVSKKYIKSLIDDNKIYLFKIYNKDFSKYSKGTPNLHTLYFKMLFDKENLENPIYKLSGNAEMFFRKGNLDLDKTTIHHANQPINNKNPNNRKKQSVFKYDIIKNRRYTVDKFALHMSITTNFQVYKNKNVNETVNRALKYCDDIYAIGIDRGERNLLYACVVNSRGEIVKQVPLNFVCNTDYHQLLAKREEERMNSRKNWKIIDNIKNLKEGYLSQAIHIITDFMVEYNAVLVLEDLNFRFKEKRMKFEKSVYQKFEKMLIDKLNFLVDKKLDKNANGGLFNAYQLTEKFTSFKDMKNQNGIVFYIPAWMTSKIDPVTGFTNLFYIKYESIEKSKEFFGKFKSIKFNKVDNYFEFEFDYNDFTDRAQGTRSKWTVCSFGPRIEGFRNPEKNNKWDGREIDITEKIKKLLDDYNISLDEDIQAQIMDINTKDFFEKLIKYFKLVLQMRNSKTGTDIDYIISPVRNKQNEFFDSRKKNEKLPMDADANGAYNIARKGLMFIDIIKETEDKDLKMPKLFIKNKDWLNYVQKSDL

SEQ ID NO: 45 - protein, 1197 aa, synthetic construct
MNIYENFTNMYQVNKTIRMGLKPICKTDENIAKFLEEDKERSEKYKIAKKIIDKENRAFIEDRLKDFSISGLDEYLELLKQKKDITKIQKKMRDEISKQLKGFPQFDSKYKFQYITDKEDTEILEYFKKFTTFFTGFNSNRENVYSKEDISTSIGHRIIHENLPKFISNFRILNKAIEALGTGKINEDFKNNEINVTVEELNKIDYFNKVLTQSGIDLYNNLIGILNQNINLYNQQQKVKKNKIGKLETLHKQILSEKDKVSFIEEFAEDNQLLKCIDEYFKEKSCLINVDLKNLLENIDTYSLNGIFIKNDKSLKNISIYLYKDWGYISNLINEEYDYKHKNKVKDDKYYEKRKKAIDKIKYFSIGYIDELLKDKNVPMVECYFKEKINLVVKEFIDYLNKFNEYKFTNELKTDEIAVEIIKNLCDSIKDVQEIVKPLIVIENDKDDSFYGEINYIWDELNKFNKIYNMVRNYLTKKDYIDEKIRLTFNRPQLLGGWSYKKEADYSSFILKDNKYYYLAIMNPNSKSKLKDLKKPENDGDTIYKMKYFQASDPAKSIPNLMVINGKTEKKNGRKDNDGENRELERLRNEYLPTEINEIRKNKAYLRSSSNFSNEALVKYIDYYKERVKEYFNEIDFKFKETCEYKQFNEFAEDVNLQAYQISFIEVSKKYIKSLIDDNKIYLFKIYNKDFSKYSKGTPNLHTLYFKMLFDKENLENPIYKLSGNAEMFFRKGNLDLDKTTIHHANQPINNKNPNNRKKQSVFKYDIIKNRRYTVDKFLLHMSITTNFQVYKNKNVNETVNRALKYCDDIYAIGIDRGERNLLYACVVNSRGEIVKQVPLNFVCNTDYHQLLAKREEERMNSRKNWKIIDNIKNLKEGYLSQAIHIITDFMVEYNAVLVLEDLNFRFKEKRMKFEKSVYQKFEKMLIDKLNFLVDKKLDKNANGGLFNAYQLTEKFTSFKDMKNQNGIVFYIPAWMTSKIDPVTGFTNLFYIKYESIEKSKEFFGKFKSIKFNKVDNYFEFEFDYNDFTDRAQGTRSKWTVCSFGPRIEGFRNPEKNNKWDGREIDITEKIKKLLDDYNISLDEDIQAQIMDINTKDFFEKLIKYFKLVLQMRNSKTGTDIDYIISPVRNKQNEFFDSRKKNEKLPMDADANGAYNIARKGLMFIDIIKETEDKDLKMPKLFIKNKDWLNYVQKSDL

SEQ ID NO: 46 - protein, 1188 aa, synthetic construct
MNIYEKFTNMYQVNKTIRMGLKPICKTDENIAKFLEEDKERSEKYKIAKKIIDKENRAFIEDRLKDFSISGLDEYLELLKQKKDITKIQKKMRDEISKQLKGFPQFDSKYKFQYITDKEDTEILEYFKKFTTFFTGFNRNRENVYSKEDISTSIGHRIIHENLPKFISNFRILNKAIEALGTGKINEDFKNNEINVTVEELNKIDYFNKVLTQSGIDLYNNLIGILNQNINLYNQQQKVKKNKIGKLETLHKQILSEKDKVSFIEEFAEDNQLLKCIDEYFKEKSCLINVDLKNLLENIDTYSLNGIFIKNDKSLKNISIYLYKDWGYISNLINEEYDYKHKNKVKDDKYYEKRKKAIDKIKYFSIGYIDELLKDKNVPMVECYFKEKINLVVKEFNASLNKFNEYKFTNELKTDEIAVEIIKNLCDSIKKIQGIIKPLIITGNDKDDDFYVEINYIWDELNKFDKIYNMVRNYLTKKDYIEEKIRMMFSKSSFMDGWGKDYGTKEAHIVYHDKNYYLVIVDEKLKLEDIDKLYKPGGDTVHYIYNYQSIDYRNIPRKFIYSKGNRFAPSVERYNLPIEDVIEVYNNKYYRTEYEEKNPKIYKKSLTSLIDYFKIGVNRDMDFEKFDIKLKDSNEYKNINEFYYNLETCCYKLQEEKVNFSVLEEFSYSGKIYLFKIYNKDFSKYSKGTPNLHTLYFKMLFDKENLENPIYKLSGNAEMFFRKGNLDLDKTTIHHANQPINNKNPNNRKRQSVFKYDIIKNRRYTVDKFALHMSITTNFQVYKNKNVNETVNRALKYCDDIYAIGIDRGERNLLYACVVNSRGEIVKQVPLNFVGNTDYHQLLAKREEERMNSRKNWKIIDNIKNLKEGYLSQAIHIITDFMVEYNAVLVLEDLNFRFKEKRMKFEKSVYQKFEKMLIDKLNFLVDKKLDKNANGGLFNAYQLTEKFTSFKDMKNQNGIVFYIPAWMTSKIDPVTGFTNLFYIKYESIEKAKEFFGKFKSIKFNKVDNYFEFEFDYNDFTDRAQGTRSKWTVCSFGPRIEGFRNPEKNNNWDGREIDITEEIKKLLDDYKVSLDEDIKAQIMDINTKDFFEKLIKYFKLVLQMRNSKTGTDIDYIISPVRNKQNEFFDSRKKNEKLPMDADANGAYNIARKGLMFIDIIKETEDKDLKMPKLFIKNKDWLNYVQKSDL

SEQ ID NO: 47 - protein, 1188 aa, synthetic construct
MNIYEKFTNMYQVNKTIRMGLKPICKTDENIAKFLEEDKERSEKYKIAKKIIDKENRAFIEDRLKDFSISGLDEYLELLKQKKDITKIQKKMRDEISKQLKGFPQFDSKYKFQYITDKEDTEILEYFKKFTTFFTGFNSNRENVYSKEDISTSIGHRIIHENLPKFISNFRILNKAIEALGTGKINEDFKNNEINVTVEELNKIDYFNKVLTQSGIDLYNNLIGILNQNINLYNQQQKVKKNKIGKLETLHKQILSEKDKVSFIEEFAEDNQLLKCIDEYFKEKSCLINVDLKNLLENIDTYSLNGIFIKNDKSLKNISIYLYKDWGYISNLINEEYDYKHKNKVKDDKYYEKRKKAIDKIKYFSIGYIDELLKDKNVPMVECYFKEKINLVVKEFNASLNKFNEYKFTNELKTDEIAVEIIKNLCDSIKKIQGIIKPLIITGNDKDDDFYVEINYIWDELNKFDKIYNMVRNYLTKKDYIEEKIRMMFSKSSFMRGWGKDYGTKEAHIVYHDKNYYLVIVDEKLKLEDIDKLYKPGGDTVHYIYNYQSIDYRNIPRKFIYSKGNRFAPSVERYNLPIEDVIEVYNNKYYRTEYEEKNPKIYKKSLTSLIDYFKIGVNRDMDFEKFDIKLKDSNEYKNINEFYYNLETCCYKLQEEKVNFSVLEEFSYSGKIYLFKIYNKDFSKYSKGTPNLHTLYFKMLFDKENLENPIYKLSGNAEMFFRKGNLDLDKTTIHHANQPINNKNPNNRKRQSVFKYDIIKNRRYTVDKFALHMSITTNFQVYKNKNVNETVNRALKYCDDIYAIGIDRGERNLLYACVVNSRGEIVKQVPLNFVGNTDYHQLLAKREEERMNSRKNWKIIDNIKNLKEGYLSQAIHIITDFMVEYNAVLVLEDLNFRFKEKRMKFEKSVYQKFEKMLIDKLNFLVDKKLDKNANGGLFNAYQLTEKFTSFKDMKNQNGIVFYIPAWMTSKIDPVTGFTNLFYIKYESIEKAKEFFGKFKSIKFNKVDNYFEFEFDYNDFTDRAQGTRSKWTVCSFGPRIEGFRNPEKNNNWDGREIDITEEIKKLLDDYKVSLDEDIKAQIMDINTKDFFEKLIKYFKLVLQMRNSKTGTDIDYIISPVRNKQNEFFDSRKKNEKLPMDADANGAYNIARKGLMFIDIIKETEDKDLKMPKLFIKNKDWLNYVQKSDL

SEQ ID NO: 48 - protein, 1188 aa, synthetic construct
MNIYEKFTNMYQVNKTIRMGLKPICKTDENIAKFLEEDKERSEKYKIAKKIIDKENRAFIEDRLKDFSISGLDEYLELLKQKKDITKIQKKMRDEISKQLKGFPQFDSKYKFQYITDKEDTEILEYFKKFTTFFTGFNSNRENVYSKEDISTSIGHRIIHENLPKFISNFRILNKAIEALGTGKINEDFKNNEINVTVEELNKIDYFNKVLTQSGIDLYNNLIGILNQNINLYNQQQKVKKNKIGKLETLHKQILSEKDKVSFIEEFAEDNQLLKCIDEYFKEKSCLINVDLKNLLENIDTYSLNGIFIKNDKSLKNISIYLYKDWGYISNLINEEYDYKHKNKVKDDKYYEKRKKAIDKIKYFSIGYIDELLKDKNVPMVECYFKEKINLVVKEFNASLNKFNEYKFTNELKTDEIAVEIIKNLCDSIKKIQGIIKPLIITGNDKDDDFYVEINYIWDELNKFDKIYNMVRNYLTKKDYIEEKIRMMFSKSSFMDGWGKDRGTKEAHIVYHDKNYYLVIVDEKLKLEDIDKLYKPGGDTVHYIYNYQSIDYRNIPRKFIYSKGNRFAPSVERYNLPIEDVIEVYNNKYYRTEYEEKNPKIYKKSLTSLIDYFKIGVNRDMDFEKFDIKLKDSNEYKNINEFYYNLETCCYKLQEEKVNFSVLEEFSYSGKIYLFKIYNKDFSKYSKGTPNLHTLYFKMLFDKENLENPIYKLSGNAEMFFRKGNLDLDKTTIHHANQPINNKNPNNRKRQSVFKYDIIKNRRYTVDKFALHMSITTNFQVYKNKNVNETVNRALKYCDDIYAIGIDRGERNLLYACVVNSRGEIVKQVPLNFVGNTDYHQLLAKREEERMNSRKNWKIIDNIKNLKEGYLSQAIHIITDFMVEYNAVLVLEDLNFRFKEKRMKFEKSVYQKFEKMLIDKLNFLVDKKLDKNANGGLFNAYQLTEKFTSFKDMKNQNGIVFYIPAWMTSKIDPVTGFTNLFYIKYESIEKAKEFFGKFKSIKFNKVDNYFEFEFDYNDFTDRAQGTRSKWTVCSFGPRIEGFRNPEKNNNWDGREIDITEEIKKLLDDYKVSLDEDIKAQIMDINTKDFFEKLIKYFKLVLQMRNSKTGTDIDYIISPVRNKQNEFFDSRKKNEKLPMDADANGAYNIARKGLMFIDIIKETEDKDLKMPKLFIKNKDWLNYVQKSDL

SEQ ID NO: 49 - protein, 1188 aa, synthetic construct
MNIYEKFTNMYQVNKTIRMGLKPICKTDENIAKFLEEDKERSEKYKIAKKIIDKENRAFIEDRLKDFSISGLDEYLELLKQKKDITKIQKKMRDEISKQLKGFPQFDSKYKFQYITDKEDTEILEYFKKFTTFFTGFNRNRENVYSKEDISTSIGHRIIHENLPKFISNFRILNKAIEALGTGKINEDFKNNEINVTVEELNKIDYFNKVLTQSGIDLYNNLIGILNQNINLYNQQQKVKKNKIGKLETLHKQILSEKDKVSFIEEFAEDNQLLKCIDEYFKEKSCLINVDLKNLLENIDTYSLNGIFIKNDKSLKNISIYLYKDWGYISNLINEEYDYKHKNKVKDDKYYEKRKKAIDKIKYFSIGYIDELLKDKNVPMVECYFKEKINLVVKEFNASLNKFNEYKFTNELKTDEIAVEIIKNLCDSIKKIQGIIKPLIITGNDKDDDFYVEINYIWDELNKFDKIYNMVRNYLTKKDYIEEKIRMMFSKSSFMRGWGKDYGTKEAHIVYHDKNYYLVIVDEKLKLEDIDKLYKPGGDTVHYIYNYQSIDYRNIPRKFIYSKGNRFAPSVERYNLPIEDVIEVYNNKYYRTEYEEKNPKIYKKSLTSLIDYFKIGVNRDMDFEKFDIKLKDSNEYKNINEFYYNLETCCYKLQEEKVNFSVLEEFSYSGKIYLFKIYNKDFSKYSKGTPNLHTLYFKMLFDKENLENPIYKLSGNAEMFFRKGNLDLDKTTIHHANQPINNKNPNNRKRQSVFKYDIIKNRRYTVDKFALHMSITTNFQVYKNKNVNETVNRALKYCDDIYAIGIDRGERNLLYACVVNSRGEIVKQVPLNFVGNTDYHQLLAKREEERMNSRKNWKIIDNIKNLKEGYLSQAIHIITDFMVEYNAVLVLEDLNFRFKEKRMKFEKSVYQKFEKMLIDKLNFLVDKKLDKNANGGLFNAYQLTEKFTSFKDMKNQNGIVFYIPAWMTSKIDPVTGFTNLFYIKYESIEKAKEFFGKFKSIKFNKVDNYFEFEFDYNDFTDRAQGTRSKWTVCSFGPRIEGFRNPEKNNNWDGREIDITEEIKKLLDDYKVSLDEDIKAQIMDINTKDFFEKLIKYFKLVLQMRNSKTGTDIDYIISPVRNKQNEFFDSRKKNEKLPMDADANGAYNIARKGLMFIDIIKETEDKDLKMPKLFIKNKDWLNYVQKSDL

SEQ ID NO: 50 - protein, 1188 aa, synthetic construct
MNIYEKFTNMYQVNKTIRMGLKPICKTDENIAKFLEEDKERSEKYKIAKKIIDKENRAFIEDRLKDFSISGLDEYLELLKQKKDITKIQKKMRDEISKQLKGFPQFDSKYKFQYITDKEDTEILEYFKKFTTFFTGFNRNRENVYSKEDISTSIGHRIIHENLPKFISNFRILNKAIEALGTGKINEDFKNNEINVTVEELNKIDYFNKVLTQSGIDLYNNLIGILNQNINLYNQQQKVKKNKIGKLETLHKQILSEKDKVSFIEEFAEDNQLLKCIDEYFKEKSCLINVDLKNLLENIDTYSLNGIFIKNDKSLKNISIYLYKDWGYISNLINEEYDYKHKNKVKDDKYYEKRKKAIDKIKYFSIGYIDELLKDKNVPMVECYFKEKINLVVKEFNASLNKFNEYKFTNELKTDEIAVEIIKNLCDSIKKIQGIIKPLIITGNDKDDDFYVEINYIWDELNKFDKIYNMVRNYLTKKDYIEEKIRMMFSKSSFMDGWGKDRGTKEAHIVYHDKNYYLVIVDEKLKLEDIDKLYKPGGDTVHYIYNYQSIDYRNIPRKFIYSKGNRFAPSVERYNLPIEDVIEVYNNKYYRTEYEEKNPKIYKKSLTSLIDYFKIGVNRDMDFEKFDIKLKDSNEYKNINEFYYNLETCCYKLQEEKVNFSVLEEFSYSGKIYLFKIYNKDFSKYSKGTPNLHTLYFKMLFDKENLENPIYKLSGNAEMFFRKGNLDLDKTTIHHANQPINNKNPNNRKRQSVFKYDIIKNRRYTVDKFALHMSITTNFQVYKNKNVNETVNRALKYCDDIYAIGIDRGERNLLYACVVNSRGEIVKQVPLNFVGNTDYHQLLAKREEERMNSRKNWKIIDNIKNLKEGYLSQAIHIITDFMVEYNAVLVLEDLNFRFKEKRMKFEKSVYQKFEKMLIDKLNFLVDKKLDKNANGGLFNAYQLTEKFTSFKDMKNQNGIVFYIPAWMTSKIDPVTGFTNLFYIKYESIEKAKEFFGKFKSIKFNKVDNYFEFEFDYNDFTDRAQGTRSKWTVCSFGPRIEGFRNPEKNNNWDGREIDITEEIKKLLDDYKVSLDEDIKAQIMDINTKDFFEKLIKYFKLVLQMRNSKTGTDIDYIISPVRNKQNEFFDSRKKNEKLPMDADANGAYNIARKGLMFIDIIKETEDKDLKMPKLFIKNKDWLNYVQKSDL

SEQ ID NO: 51 - protein, 1188 aa, synthetic construct
MNIYEKFTNMYQVNKTIRMGLKPICKTDENIAKFLEEDKERSEKYKIAKKIIDKENRAFIEDRLKDFSISGLDEYLELLKQKKDITKIQKKMRDEISKQLKGFPQFDSKYKFQYITDKEDTEILEYFKKFTTFFTGFNSNRENVYSKEDISTSIGHRIIHENLPKFISNFRILNKAIEALGTGKINEDFKNNEINVTVEELNKIDYFNKVLTQSGIDLYNNLIGILNQNINLYNQQQKVKKNKIGKLETLHKQILSEKDKVSFIEEFAEDNQLLKCIDEYFKEKSCLINVDLKNLLENIDTYSLNGIFIKNDKSLKNISIYLYKDWGYISNLINEEYDYKHKNKVKDDKYYEKRKKAIDKIKYFSIGYIDELLKDKNVPMVECYFKEKINLVVKEFNASLNKFNEYKFTNELKTDEIAVEIIKNLCDSIKKIQGIIKPLIITGNDKDDDFYVEINYIWDELNKFDKIYNMVRNYLTKKDYIEEKIRMMFSKSSFMRGWGKDRGTKEAHIVYHDKNYYLVIVDEKLKLEDIDKLYKPGGDTVHYIYNYQSIDYRNIPRKFIYSKGNRFAPSVERYNLPIEDVIEVYNNKYYRTEYEEKNPKIYKKSLTSLIDYFKIGVNRDMDFEKFDIKLKDSNEYKNINEFYYNLETCCYKLQEEKVNFSVLEEFSYSGKIYLFKIYNKDFSKYSKGTPNLHTLYFKMLFDKENLENPIYKLSGNAEMFFRKGNLDLDKTTIHHANQPINNKNPNNRKRQSVFKYDIIKNRRYTVDKFALHMSITTNFQVYKNKNVNETVNRALKYCDDIYAIGIDRGERNLLYACVVNSRGEIVKQVPLNFVGNTDYHQLLAKREEERMNSRKNWKIIDNIKNLKEGYLSQAIHIITDFMVEYNAVLVLEDLNFRFKEKRMKFEKSVYQKFEKMLIDKLNFLVDKKLDKNANGGLFNAYQLTEKFTSFKDMKNQNGIVFYIPAWMTSKIDPVTGFTNLFYIKYESIEKAKEFFGKFKSIKFNKVDNYFEFEFDYNDFTDRAQGTRSKWTVCSFGPRIEGFRNPEKNNNWDGREIDITEEIKKLLDDYKVSLDEDIKAQIMDINTKDFFEKLIKYFKLVLQMRNSKTGTDIDYIISPVRNKQNEFFDSRKKNEKLPMDADANGAYNIARKGLMFIDIIKETEDKDLKMPKLFIKNKDWLNYVQKSDL

SEQ ID NO: 52 - protein, 1188 aa, synthetic construct
MNIYEKFTNMYQVNKTIRMGLKPICKTDENIAKFLEEDKERSEKYKIAKKIIDKENRAFIEDRLKDFSISGLDEYLELLKQKKDITKIQKKMRDEISKQLKGFPQFDSKYKFQYITDKEDTEILEYFKKFTTFFTGFNRNRENVYSKEDISTSIGHRIIHENLPKFISNFRILNKAIEALGTGKINEDFKNNEINVTVEELNKIDYFNKVLTQSGIDLYNNLIGILNQNINLYNQQQKVKKNKIGKLETLHKQILSEKDKVSFIEEFAEDNQLLKCIDEYFKEKSCLINVDLKNLLENIDTYSLNGIFIKNDKSLKNISIYLYKDWGYISNLINEEYDYKHKNKVKDDKYYEKRKKAIDKIKYFSIGYIDELLKDKNVPMVECYFKEKINLVVKEFNASLNKFNEYKFTNELKTDEIAVEIIKNLCDSIKKIQGIIKPLIITGNDKDDDFYVEINYIWDELNKFDKIYNMVRNYLTKKDYIEEKIRMMFSKSSFMRGWGKDRGTKEAHIVYHDKNYYLVIVDEKLKLEDIDKLYKPGGDTVHYIYNYQSIDYRNIPRKFIYSKGNRFAPSVERYNLPIEDVIEVYNNKYYRTEYEEKNPKIYKKSLTSLIDYFKIGVNRDMDFEKFDIKLKDSNEYKNINEFYYNLETCCYKLQEEKVNFSVLEEFSYSGKIYLFKIYNKDFSKYSKGTPNLHTLYFKMLFDKENLENPIYKLSGNAEMFFRKGNLDLDKTTIHHANQPINNKNPNNRKRQSVFKYDIIKNRRYTVDKFALHMSITTNFQVYKNKNVNETVNRALKYCDDIYAIGIDRGERNLLYACVVNSRGEIVKQVPLNFVGNTDYHQLLAKREEERMNSRKNWKIIDNIKNLKEGYLSQAIHIITDFMVEYNAVLVLEDLNFRFKEKRMKFEKSVYQKFEKMLIDKLNFLVDKKLDKNANGGLFNAYQLTEKFTSFKDMKNQNGIVFYIPAWMTSKIDPVTGFTNLFYIKYESIEKAKEFFGKFKSIKFNKVDNYFEFEFDYNDFTDRAQGTRSKWTVCSFGPRIEGFRNPEKNNNWDGREIDITEEIKKLLDDYKVSLDEDIKAQIMDINTKDFFEKLIKYFKLVLQMRNSKTGTDIDYIISPVRNKQNEFFDSRKKNEKLPMDADANGAYNIARKGLMFIDIIKETEDKDLKMPKLFIKNKDWLNYVQKSDL

SEQ ID NO: 53 - protein, 1188 aa, synthetic construct
MNIYEKFTNMYQVNKTIRMGLKPICKTDENIAKFLEEDKERSEKYKIAKKIIDKENRAFIEDRLKDFSISGLDEYLELLKQKKDITKIQKKMRDEISKQLKGFPQFDSKYKFQYITDKEDTEILEYFKKFTTFFTGFNRNRENVYSKEDISTSIGHRIIHENLPKFISNFRILNKAIEALGTGKINEDFKNNEINVTVEELNKIDYFNKVLTQSGIDLYNNLIGILNQNIALYNQQQKVKKNKIGKLETLHKQILSEKDKVSFIEEFAEDNQLLKCIDEYFKEKSCLINVDLKNLLENIDTYSLNGIFIKNDKSLKNISIYLYKDWGYISNLINEEYDYKHKNKVKDDKYYEKRKKAIDKIKYFSIGYIDELLKDKNVPMVECYFKEKINLVVKEFNASLNKFNEYKFTNELKTDEIAVEIIKNLCDSIKKIQGIIKPLIITGNDKDDDFYVEINYIWDELNKFDKIYNMVRNYLTKKDYIEEKIRMMFSKSSFMDGWGKDRGTKEAHIVYHDKNYYLVIVDEKLKLEDIDKLYKPGGDTVHYIYNYQSIDYRNIPRKFIYSKGNRFAPSVERYNLPIEDVIEVYNNKYYRTEYEEKNPKIYKKSLTSLIDYFKIGVNRDMDFEKFDIKLKDSNEYKNINEFYYNLETCCYKLQEEKVNFSVLEEFSYSGKIYLFKIYNKDFSKYSKGTPNLHTLYFKMLFDKENLENPIYKLSGNAEMFFRKGNLDLDKTTIHHANQPINNKNPNNRKRQSVFKYDIIKNRRYTVDKFALHMSITTNFQVYKNKNVNETVNRALKYCDDIYAIGIDRGERNLLYACVVNSRGEIVKQVPLNFVGNTDYHQLLAKREEERMNSRKNWKIIDNIKNLKEGYLSQAIHIITDFMVEYNAVLVLEDLNFRFKEKRMKFEKSVYQKFEKMLIDKLNFLVDKKLDKNANGGLFNAYQLTEKFTSFKDMKNQNGIVFYIPAWMTSKIDPVTGFTNLFYIKYESIEKAKEFFGKFKSIKFNKVDNYFEFEFDYNDFTDRAQGTRSKWTVCSFGPRIEGFRNPEKNNNWDGREIDITEEIKKLLDDYKVSLDEDIKAQIMDINTKDFFEKLIKYFKLVLQMRNSKTGTDIDYIISPVRNKQNEFFDSRKKNEKLPMDADANGAYNIARKGLMFIDIIKETEDKDLKMPKLFIKNKDWLNYVQKSDL

SEQ ID NO: 54 - protein, 1188 aa, synthetic construct
MNIYEKFTNMYQVNKTIRMGLKPICKTDENIAKFLEEDKERSEKYKIAKKIIDKENRAFIEDRLKDFSISGLDEYLELLKQKKDITKIQKKMRDEISKQLKGFPQFDSKYKFQYITDKEDTEILEYFKKFTTFFTGFNSNRENVYSKEDISTSIGHRIIHENLPKFISNFRILNKAIEALGTGKINEDFKNNEINVTVEELNKIDYFNKVLTQSGIDLYNNLIGILNQNINLYNQQQKVKKNKIGKLETLHKQILSEKDKVSFIEEFAEDNQLLKCIDEYFKEKSCLINVDLKNLLENIDTYSLNGIFIKNDKSLKNISIYLYKDWGYISNLINEEYDYKHKNKVKDDKYYEKRKKAIDKIKYFSIGYIDELLKDKNVPMVECYFKEKINLVVKEFNASLNKFNEYKFTNELKTDEIAVEIIKNLCDSIKKIQGIIKPLIITGNDKDDDFYVEINYIWDELNKFDKIYNMVRNYLTKKDYIEEKIRMMFSRSSFMDGWGKDYGTKEAHIVYHDKNYYLVIVDEKLKLEDIDKLYKPGGDTVHYIYNYQSIDYRNIPRKFIYSKGNRFAPSVERYNLPIEDVIEVYNNKYYRTEYEEKNPKIYKKSLTSLIDYFKIGVNRDMDFEKFDIKLKDSNEYKNINEFYYNLETCCYKLQEEKVNFSVLEEFSYSGKIYLFKIYNKDFSKYSKGTPNLHTLYFKMLFDKENLENPIYKLSGNAEMFFRKGNLDLDKTTIHHANQPINNKNPNNRKRQSVFKYDIIKNRRYTVDKFALHMSITTNFQVYKNKNVNETVNRALKYCDDIYAIGIDRGERNLLYACVVNSRGEIVKQVPLNFVGNTDYHQLLAKREEERMNSRKNWKIIDNIKNLKEGYLSQAIHIITDFMVEYNAVLVLEDLNFRFKEKRMKFEKSVYQKFEKMLIDKLNFLVDKKLDKNANGGLFNAYQLTEKFTSFKDMKNQNGIVFYIPAWMTSKIDPVTGFTNLFYIKYESIEKAKEFFGKFKSIKFNKVDNYFEFEFDYNDFTDRAQGTRSKWTVCSFGPRIEGFRNPEKNNNWDGREIDITEEIKKLLDDYKVSLDEDIKAQIMDINTKDFFEKLIKYFKLVLQMRNSKTGTDIDYIISPVRNKQNEFFDSRKKNEKLPMDADANGAYNIARKGLMFIDIIKETEDKDLKMPKLFIKNKDWLNYVQKSDL

SEQ ID NO: 55 - protein, 1198 aa, synthetic construct
MTSKNSFEDFTNLYELSKTLRFELKPVGETLNSLEKDGVIQKDKEVEENYNKIKVFFDSLHREFVKQSLEKGYLNEIDNFYNSYIELNKTPENKKNKTLQKDFEKASKILKKELVSFFESKGNEWKQKYSFFKKGGIDVLNEKEVLDLMGEFYPEQKELFKKFDRFFTYFSNFKRSRKNFYSEDGRAGAVATRAIDENLITFIKNLEDFKRFQNNFSDFISQNFSQEEKQAFELDFYNSCLLQDGIDNYNKILGGYSLENGNKIQGINEKINLFKQNQNKSNSKDIKFPRFKLLYKQILSDKEKIAFIDEIESDEKLLDFVKNSLEKNKFKIIEAEKIINNFIKNNPSFELDKIYLSKVSINTISNKFFASWDYILKEGFEKGEIKDFVSFEELKNAFEKIKYEKLEDIFKANYISENIVLEGESLYKNFLHIFFNEFQKNISDINLYTSKIEKLLGEKFEKTEIQVETIKNYFDSVLSLYKMTKYFALEKGKSKVDLETDNNFYNDFFAYYEDFEIWKNYNLVRNFVTKKQIQTDKFKLNFNNSQFLTGWDKDKEKERLGIILKKEDKFYLGVFKNNKILDNYKYESGDFYEKMNYKQLNNVYRQLPRFAFSNSKKEVYGITPELKKIKEEFDIFQKSKEKGEKFDPEKLKKIIECYKRGFIATYENEFDLEKIKNTDYTDLPTFYDDIEKKTYKLQFSKINSDFIEEKVKSSDLYLFQIYNKDFSEFKKAGSKENIHTKYFKLLFDKKNLENLVIKLSGGAEIFFREKTEKLKTKLDKSGKEVLDHRRYGQDKTMLHLSITLNANTGDSYGFNKMINEYLNKNEDIKIIGIDRGEKHLAYYSVIDKTGKIYDIGSLNEINGVNYLVKLEKIELSRKDARVSWGEIENIKELKNGYISQVINKLAELITKHNAVIVFEDLNQGFKRGRQKIEKQIYQKLELALAKKLNYLTQKDKGDDEVLGNLKALQLVPKVNDYQDIANYKQSGIMFYTRANYTSTTCPCCGFRKNIYISNSDTKEKQIKAFEKINIKFDGKKFIFSYEMEQDKKTKQKSNKTSFIVNSNFSRFKYNTLTKEIDNINITEELKRIFENIDLKGNINEQIKVLKGFSFSELTKKFNLLLQLRNSDTKNALDYIICPACNYHSKDGFQGLDYNADANGAYNIARKGIIMIERIKNIFEKPDLYVSDIDWDNFTQKTQ

SEQ ID NO: 56 - protein, 1198 aa, synthetic construct
MTSKNSFEDFTNLYELSKTLRFELKPVGETLNSLEKDGVIQKDKEVEENYNKIKVFFDSLHREFVKQSLEKGYLNEIDNFYNSYIELNKTPENKKNKTLQKDFEKASKILKKELVSFFESKGNEWKQKYSFFKKGGIDVLNEKEVLDLMGEFYPEQKELFKKFDRFFTYFSNFKESRKNFYSEDGRAGAVATRAIDENLITFIKNLEDFKRFQNNFSDFISQNFSQEEKQAFELDFYNSCLLQDGIDNYNKILGGYSLENGNKIQGINEKINLFKQNQNKSNSKDIKFPRFKLLYKQILSDKEKIAFIDEIESDEKLLDFVKNSLEKNKFKIIEAEKIINNFIKNNPSFELDKIYLSKVSINTISNKFFASWDYILKEGFEKGEIKDFVSFEELKNAFEKIKYEKLEDIFKANYISENIVLEGESLYKNFLHIFFNEFQKNISDINLYTSKIEKLLGEKFEKTEIQVETIKNYFDSVLSLYKMTKYFALEKGKSKVDLETDNNFYNDFFAYYEDFEIWKNYNLVRNFVTKKQIQTDKFKLNFNNSQFLRGWDKDKEKERLGIILKKEDKFYLGVFKNNKILDNYKYESGDFYEKMNYKQLNNVYRQLPRFAFSNSKKEVYGITPELKKIKEEFDIFQKSKEKGEKFDPEKLKKIIECYKRGFIATYENEFDLEKIKNTDYTDLPTFYDDIEKKTYKLQFSKINSDFIEEKVKSSDLYLFQIYNKDFSEFKKAGSKENIHTKYFKLLFDKKNLENLVIKLSGGAEIFFREKTEKLKTKLDKSGKEVLDHRRYGQDKTMLHLSITLNANTGDSYGFNKMINEYLNKNEDIKIIGIDRGEKHLAYYSVIDKTGKIYDIGSLNEINGVNYLVKLEKIELSRKDARVSWGEIENIKELKNGYISQVINKLAELITKHNAVIVFEDLNQGFKRGRQKIEKQIYQKLELALAKKLNYLTQKDKGDDEVLGNLKALQLVPKVNDYQDIANYKQSGIMFYTRANYTSTTCPCCGFRKNIYISNSDTKEKQIKAFEKINIKFDGKKFIFSYEMEQDKKTKQKSNKTSFIVNSNFSRFKYNTLTKEIDNINITEELKRIFENIDLKGNINEQIKVLKGFSFSELTKKFNLLLQLRNSDTKNALDYIICPACNYHSKDGFQGLDYNADANGAYNIARKGIIMIERIKNIFEKPDLYVSDIDWDNFTQKTQ

SEQ ID NO: 57 - protein, 1198 aa, synthetic construct
MTSKNSFEDFTNLYELSKTLRFELKPVGETLNSLEKDGVIQKDKEVEENYNKIKVFFDSLHREFVKQSLEKGYLNEIDNFYNSYIELNKTPENKKNKTLQKDFEKASKILKKELVSFFESKGNEWKQKYSFFKKGGIDVLNEKEVLDLMGEFYPEQKELFKKFDRFFTYFSNFKESRKNFYSEDGRAGAVATRAIDENLITFIKNLEDFKRFQNNFSDFISQNFSQEEKQAFELDFYNSCLLQDGIDNYNKILGGYSLENGNKIQGINEKINLFKQNQNKSNSKDIKFPRFKLLYKQILSDKEKIAFIDEIESDEKLLDFVKNSLEKNKFKIIEAEKIINNFIKNNPSFELDKIYLSKVSINTISNKFFASWDYILKEGFEKGEIKDFVSFEELKNAFEKIKYEKLEDIFKANYISENIVLEGESLYKNFLHIFFNEFQKNISDINLYTSKIEKLLGEKFEKTEIQVETIKNYFDSVLSLYKMTKYFALEKGKSKVDLETDNNFYNDFFAYYEDFEIWKNYNLVRNFVTKKQIQTDKFKLNFNNSQFLTGWDKDREKERLGIILKKEDKFYLGVFKNNKILDNYKYESGDFYEKMNYKQLNNVYRQLPRFAFSNSKKEVYGITPELKKIKEEFDIFQKSKEKGEKFDPEKLKKIIECYKRGFIATYENEFDLEKIKNTDYTDLPTFYDDIEKKTYKLQFSKINSDFIEEKVKSSDLYLFQIYNKDFSEFKKAGSKENIHTKYFKLLFDKKNLENLVIKLSGGAEIFFREKTEKLKTKLDKSGKEVLDHRRYGQDKTMLHLSITLNANTGDSYGFNKMINEYLNKNEDIKIIGIDRGEKHLAYYSVIDKTGKIYDIGSLNEINGVNYLVKLEKIELSRKDARVSWGEIENIKELKNGYISQVINKLAELITKHNAVIVFEDLNQGFKRGRQKIEKQIYQKLELALAKKLNYLTQKDKGDDEVLGNLKALQLVPKVNDYQDIANYKQSGIMFYTRANYTSTTCPCCGFRKNIYISNSDTKEKQIKAFEKINIKFDGKKFIFSYEMEQDKKTKQKSNKTSFIVNSNFSRFKYNTLTKEIDNINITEELKRIFENIDLKGNINEQIKVLKGFSFSELTKKFNLLLQLRNSDTKNALDYIICPACNYHSKDGFQGLDYNADANGAYNIARKGIIMIERIKNIFEKPDLYVSDIDWDNFTQKTQ

SEQ ID NO: 58 - protein, 1198 aa, synthetic construct
MTSKNSFEDFTNLYELSKTLRFELKPVGETLNSLEKDGVIQKDKEVEENYNKIKVFFDSLHREFVKQSLEKGYLNEIDNFYNSYIELNKTPENKKNKTLQKDFEKASKILKKELVSFFESKGNEWKQKYSFFKKGGIDVLNEKEVLDLMGEFYPEQKELFKKFDRFFTYFSNFKRSRKNFYSEDGRAGAVATRAIDENLITFIKNLEDFKRFQNNFSDFISQNFSQEEKQAFELDFYNSCLLQDGIDNYNKILGGYSLENGNKIQGINEKINLFKQNQNKSNSKDIKFPRFKLLYKQILSDKEKIAFIDEIESDEKLLDFVKNSLEKNKFKIIEAEKIINNFIKNNPSFELDKIYLSKVSINTISNKFFASWDYILKEGFEKGEIKDFVSFEELKNAFEKIKYEKLEDIFKANYISENIVLEGESLYKNFLHIFFNEFQKNISDINLYTSKIEKLLGEKFEKTEIQVETIKNYFDSVLSLYKMTKYFALEKGKSKVDLETDNNFYNDFFAYYEDFEIWKNYNLVRNFVTKKQIQTDKFKLNFNNSQFLRGWDKDKEKERLGIILKKEDKFYLGVFKNNKILDNYKYESGDFYEKMNYKQLNNVYRQLPRFAFSNSKKEVYGITPELKKIKEEFDIFQKSKEKGEKFDPEKLKKIIECYKRGFIATYENEFDLEKIKNTDYTDLPTFYDDIEKKTYKLQFSKINSDFIEEKVKSSDLYLFQIYNKDFSEFKKAGSKENIHTKYFKLLFDKKNLENLVIKLSGGAEIFFREKTEKLKTKLDKSGKEVLDHRRYGQDKTMLHLSITLNANTGDSYGFNKMINEYLNKNEDIKIIGIDRGEKHLAYYSVIDKTGKIYDIGSLNEINGVNYLVKLEKIELSRKDARVSWGEIENIKELKNGYISQVINKLAELITKHNAVIVFEDLNQGFKRGRQKIEKQIYQKLELALAKKLNYLTQKDKGDDEVLGNLKALQLVPKVNDYQDIANYKQSGIMFYTRANYTSTTCPCCGFRKNIYISNSDTKEKQIKAFEKINIKFDGKKFIFSYEMEQDKKTKQKSNKTSFIVNSNFSRFKYNTLTKEIDNINITEELKRIFENIDLKGNINEQIKVLKGFSFSELTKKFNLLLQLRNSDTKNALDYIICPACNYHSKDGFQGLDYNADANGAYNIARKGIIMIERIKNIFEKPDLYVSDIDWDNFTQKTQ

SEQ ID NO: 59 - protein, 1198 aa, synthetic construct
MTSKNSFEDFTNLYELSKTLRFELKPVGETLNSLEKDGVIQKDKEVEENYNKIKVFFDSLHREFVKQSLEKGYLNEIDNFYNSYIELNKTPENKKNKTLQKDFEKASKILKKELVSFFESKGNEWKQKYSFFKKGGIDVLNEKEVLDLMGEFYPEQKELFKKFDRFFTYFSNFKRSRKNFYSEDGRAGAVATRAIDENLITFIKNLEDFKRFQNNFSDFISQNFSQEEKQAFELDFYNSCLLQDGIDNYNKILGGYSLENGNKIQGINEKINLFKQNQNKSNSKDIKFPRFKLLYKQILSDKEKIAFIDEIESDEKLLDFVKNSLEKNKFKIIEAEKIINNFIKNNPSFELDKIYLSKVSINTISNKFFASWDYILKEGFEKGEIKDFVSFEELKNAFEKIKYEKLEDIFKANYISENIVLEGESLYKNFLHIFFNEFQKNISDINLYTSKIEKLLGEKFEKTEIQVETIKNYFDSVLSLYKMTKYFALEKGKSKVDLETDNNFYNDFFAYYEDFEIWKNYNLVRNFVTKKQIQTDKFKLNFNNSQFLTGWDKDREKERLGIILKKEDKFYLGVFKNNKILDNYKYESGDFYEKMNYKQLNNVYRQLPRFAFSNSKKEVYGITPELKKIKEEFDIFQKSKEKGEKFDPEKLKKIIECYKRGFIATYENEFDLEKIKNTDYTDLPTFYDDIEKKTYKLQFSKINSDFIEEKVKSSDLYLFQIYNKDFSEFKKAGSKENIHTKYFKLLFDKKNLENLVIKLSGGAEIFFREKTEKLKTKLDKSGKEVLDHRRYGQDKTMLHLSITLNANTGDSYGFNKMINEYLNKNEDIKIIGIDRGEKHLAYYSVIDKTGKIYDIGSLNEINGVNYLVKLEKIELSRKDARVSWGEIENIKELKNGYISQVINKLAELITKHNAVIVFEDLNQGFKRGRQKIEKQIYQKLELALAKKLNYLTQKDKGDDEVLGNLKALQLVPKVNDYQDIANYKQSGIMFYTRANYTSTTCPCCGFRKNIYISNSDTKEKQIKAFEKINIKFDGKKFIFSYEMEQDKKTKQKSNKTSFIVNSNFSRFKYNTLTKEIDNINITEELKRIFENIDLKGNINEQIKVLKGFSFSELTKKFNLLLQLRNSDTKNALDYIICPACNYHSKDGFQGLDYNADANGAYNIARKGIIMIERIKNIFEKPDLYVSDIDWDNFTQKTQ

SEQ ID NO: 60 - protein, 1198 aa, synthetic construct
MTSKNSFEDFTNLYELSKTLRFELKPVGETLNSLEKDGVIQKDKEVEENYNKIKVFFDSLHREFVKQSLEKGYLNEIDNFYNSYIELNKTPENKKNKTLQKDFEKASKILKKELVSFFESKGNEWKQKYSFFKKGGIDVLNEKEVLDLMGEFYPEQKELFKKFDRFFTYFSNFKESRKNFYSEDGRAGAVATRAIDENLITFIKNLEDFKRFQNNFSDFISQNFSQEEKQAFELDFYNSCLLQDGIDNYNKILGGYSLENGNKIQGINEKINLFKQNQNKSNSKDIKFPRFKLLYKQILSDKEKIAFIDEIESDEKLLDFVKNSLEKNKFKIIEAEKIINNFIKNNPSFELDKIYLSKVSINTISNKFFASWDYILKEGFEKGEIKDFVSFEELKNAFEKIKYEKLEDIFKANYISENIVLEGESLYKNFLHIFFNEFQKNISDINLYTSKIEKLLGEKFEKTEIQVETIKNYFDSVLSLYKMTKYFALEKGKSKVDLETDNNFYNDFFAYYEDFEIWKNYNLVRNFVTKKQIQTDKFKLNFNNSQFLRGWDKDREKERLGIILKKEDKFYLGVFKNNKILDNYKYESGDFYEKMNYKQLNNVYRQLPRFAFSNSKKEVYGITPELKKIKEEFDIFQKSKEKGEKFDPEKLKKIIECYKRGFIATYENEFDLEKIKNTDYTDLPTFYDDIEKKTYKLQFSKINSDFIEEKVKSSDLYLFQIYNKDFSEFKKAGSKENIHTKYFKLLFDKKNLENLVIKLSGGAEIFFREKTEKLKTKLDKSGKEVLDHRRYGQDKTMLHLSITLNANTGDSYGFNKMINEYLNKNEDIKIIGIDRGEKHLAYYSVIDKTGKIYDIGSLNEINGVNYLVKLEKIELSRKDARVSWGEIENIKELKNGYISQVINKLAELITKHNAVIVFEDLNQGFKRGRQKIEKQIYQKLELALAKKLNYLTQKDKGDDEVLGNLKALQLVPKVNDYQDIANYKQSGIMFYTRANYTSTTCPCCGFRKNIYISNSDTKEKQIKAFEKINIKFDGKKFIFSYEMEQDKKTKQKSNKTSFIVNSNFSRFKYNTLTKEIDNINITEELKRIFENIDLKGNINEQIKVLKGFSFSELTKKFNLLLQLRNSDTKNALDYIICPACNYHSKDGFQGLDYNADANGAYNIARKGIIMIERIKNIFEKPDLYVSDIDWDNFTQKTQ

SEQ ID NO: 61 - protein, 1198 aa, synthetic construct
MTSKNSFEDFTNLYELSKTLRFELKPVGETLNSLEKDGVIQKDKEVEENYNKIKVFFDSLHREFVKQSLEKGYLNEIDNFYNSYIELNKTPENKKNKTLQKDFEKASKILKKELVSFFESKGNEWKQKYSFFKKGGIDVLNEKEVLDLMGEFYPEQKELFKKFDRFFTYFSNFKRSRKNFYSEDGRAGAVATRAIDENLITFIKNLEDFKRFQNNFSDFISQNFSQEEKQAFELDFYNSCLLQDGIDNYNKILGGYSLENGNKIQGINEKINLFKQNQNKSNSKDIKFPRFKLLYKQILSDKEKIAFIDEIESDEKLLDFVKNSLEKNKFKIIEAEKIINNFIKNNPSFELDKIYLSKVSINTISNKFFASWDYILKEGFEKGEIKDFVSFEELKNAFEKIKYEKLEDIFKANYISENIVLEGESLYKNFLHIFFNEFQKNISDINLYTSKIEKLLGEKFEKTEIQVETIKNYFDSVLSLYKMTKYFALEKGKSKVDLETDNNFYNDFFAYYEDFEIWKNYNLVRNFVTKKQIQTDKFKLNFNNSQFLRGWDKDREKERLGIILKKEDKFYLGVFKNNKILDNYKYESGDFYEKMNYKQLNNVYRQLPRFAFSNSKKEVYGITPELKKIKEEFDIFQKSKEKGEKFDPEKLKKIIECYKRGFIATYENEFDLEKIKNTDYTDLPTFYDDIEKKTYKLQFSKINSDFIEEKVKSSDLYLFQIYNKDFSEFKKAGSKENIHTKYFKLLFDKKNLENLVIKLSGGAEIFFREKTEKLKTKLDKSGKEVLDHRRYGQDKTMLHLSITLNANTGDSYGFNKMINEYLNKNEDIKIIGIDRGEKHLAYYSVIDKTGKIYDIGSLNEINGVNYLVKLEKIELSRKDARVSWGEIENIKELKNGYISQVINKLAELITKHNAVIVFEDLNQGFKRGRQKIEKQIYQKLELALAKKLNYLTQKDKGDDEVLGNLKALQLVPKVNDYQDIANYKQSGIMFYTRANYTSTTCPCCGFRKNIYISNSDTKEKQIKAFEKINIKFDGKKFIFSYEMEQDKKTKQKSNKTSFIVNSNFSRFKYNTLTKEIDNINITEELKRIFENIDLKGNINEQIKVLKGFSFSELTKKFNLLLQLRNSDTKNALDYIICPACNYHSKDGFQGLDYNADANGAYNIARKGIIMIERIKNIFEKPDLYVSDIDWDNFTQKTQ

SEQ ID NO: 62 - protein, 1198 aa, synthetic construct
MTSKNSFEDFTNLYELSKTLRFELKPVGETLNSLEKDGVIQKDKEVEENYNKIKVFFDSLHREFVKQSLEKGYLNEIDNFYNSYIELNKTPENKKNKTLQKDFEKASKILKKELVSFFESKGNEWKQKYSFFKKGGIDVLNEKEVLDLMGEFYPEQKELFKKFDRFFTYFSNFKRSRKNFYSEDGRAGAVATRAIDENLITFIKNLEDFKRFQNNFSDFISQNFSQEEKQAFELDFYNSCLLQDGIDNYNKILGGYSLENGNKIQGINEKIALFKQNQNKSNSKDIKFPRFKLLYKQILSDKEKIAFIDEIESDEKLLDFVKNSLEKNKFKIIEAEKIINNFIKNNPSFELDKIYLSKVSINTISNKFFASWDYILKEGFEKGEIKDFVSFEELKNAFEKIKYEKLEDIFKANYISENIVLEGESLYKNFLHIFFNEFQKNISDINLYTSKIEKLLGEKFEKTEIQVETIKNYFDSVLSLYKMTKYFALEKGKSKVDLETDNNFYNDFFAYYEDFEIWKNYNLVRNFVTKKQIQTDKFKLNFNNSQFLTGWDKDREKERLGIILKKEDKFYLGVFKNNKILDNYKYESGDFYEKMNYKQLNNVYRQLPRFAFSNSKKEVYGITPELKKIKEEFDIFQKSKEKGEKFDPEKLKKIIECYKRGFIATYENEFDLEKIKNTDYTDLPTFYDDIEKKTYKLQFSKINSDFIEEKVKSSDLYLFQIYNKDFSEFKKAGSKENIHTKYFKLLFDKKNLENLVIKLSGGAEIFFREKTEKLKTKLDKSGKEVLDHRRYGQDKTMLHLSITLNANTGDSYGFNKMINEYLNKNEDIKIIGIDRGEKHLAYYSVIDKTGKIYDIGSLNEINGVNYLVKLEKIELSRKDARVSWGEIENIKELKNGYISQVINKLAELITKHNAVIVFEDLNQGFKRGRQKIEKQIYQKLELALAKKLNYLTQKDKGDDEVLGNLKALQLVPKVNDYQDIANYKQSGIMFYTRANYTSTTCPCCGFRKNIYISNSDTKEKQIKAFEKINIKFDGKKFIFSYEMEQDKKTKQKSNKTSFIVNSNFSRFKYNTLTKEIDNINITEELKRIFENIDLKGNINEQIKVLKGFSFSELTKKFNLLLQLRNSDTKNALDYIICPACNYHSKDGFQGLDYNADANGAYNIARKGIIMIERIKNIFEKPDLYVSDIDWDNFTQKTQ

SEQ ID NO: 63 - protein, 1198 aa, synthetic construct
MTSKNSFEDFTNLYELSKTLRFELKPVGETLNSLEKDGVIQKDKEVEENYNKIKVFFDSLHREFVKQSLEKGYLNEIDNFYNSYIELNKTPENKKNKTLQKDFEKASKILKKELVSFFESKGNEWKQKYSFFKKGGIDVLNEKEVLDLMGEFYPEQKELFKKFDRFFTYFSNFKESRKNFYSEDGRAGAVATRAIDENLITFIKNLEDFKRFQNNFSDFISQNFSQEEKQAFELDFYNSCLLQDGIDNYNKILGGYSLENGNKIQGINEKINLFKQNQNKSNSKDIKFPRFKLLYKQILSDKEKIAFIDEIESDEKLLDFVKNSLEKNKFKIIEAEKIINNFIKNNPSFELDKIYLSKVSINTISNKFFASWDYILKEGFEKGEIKDFVSFEELKNAFEKIKYEKLEDIFKANYISENIVLEGESLYKNFLHIFFNEFQKNISDINLYTSKIEKLLGEKFEKTEIQVETIKNYFDSVLSLYKMTKYFALEKGKSKVDLETDNNFYNDFFAYYEDFEIWKNYNLVRNFVTKKQIQTDKFKLNFNRSQFLTGWDKDKEKERLGIILKKEDKFYLGVFKNNKILDNYKYESGDFYEKMNYKQLNNVYRQLPRFAFSNSKKEVYGITPELKKIKEEFDIFQKSKEKGEKFDPEKLKKIIECYKRGFIATYENEFDLEKIKNTDYTDLPTFYDDIEKKTYKLQFSKINSDFIEEKVKSSDLYLFQIYNKDFSEFKKAGSKENIHTKYFKLLFDKKNLENLVIKLSGGAEIFFREKTEKLKTKLDKSGKEVLDHRRYGQDKTMLHLSITLNANTGDSYGFNKMINEYLNKNEDIKIIGIDRGEKHLAYYSVIDKTGKIYDIGSLNEINGVNYLVKLEKIELSRKDARVSWGEIENIKELKNGYISQVINKLAELITKHNAVIVFEDLNQGFKRGRQKIEKQIYQKLELALAKKLNYLTQKDKGDDEVLGNLKALQLVPKVNDYQDIANYKQSGIMFYTRANYTSTTCPCCGFRKNIYISNSDTKEKQIKAFEKINIKFDGKKFIFSYEMEQDKKTKQKSNKTSFIVNSNFSRFKYNTLTKEIDNINITEELKRIFENIDLKGNINEQIKVLKGFSFSELTKKFNLLLQLRNSDTKNALDYIICPACNYHSKDGFQGLDYNADANGAYNIARKGIIMIERIKNIFEKPDLYVSDIDWDNFTQKTQ

SEQ ID NO: 64 - protein, 1193 aa, synthetic construct
MKTFENFTNLYSLPRTLRFELKPLYKTKELIDSKQELFPKDKRIDEIYQNIIKPCLNELHSDFIEKSMENKDFQNIPDNILKIYSNEKNIDDFKNIEKDLIKQINGFLKSNKTFFAENYSDLLGKNSIDIIIKVFGEKIYKKDDSGKIFLYNDLLGKSYEELINIYFKGFSTYLSNFNRNRENLYDKKNEAKVGSVSGRTIGENFPRFLQNCINFRDKLEKLNLSIEQKDIFITNNFGKCISQKQIDYYNKIIGQINSKTNEFNQKNGLKGNKKLPKLLLLHKQILGKSENENILNFINNIIQTDFELEQEIKIINKDIFQRIDFIKKSIVSNIEDFELEKIFIKKNRLKDISSLLMDNYSVLEKLLPEFNEEGKIIKENELVNLSKIKKSFENIDLKDLKNIFKKEYFDESKDGFKLFLNGIYNHFSDLENNIKHTHKLVQDKLISGNFSENIQKSEKNINLRDEIFVSSKGLLKAYLDSILALDRFVHMFDYGEQKDFDSNFYNNIEEYSINFSPFKTYNAVRNYLTKKNYSTDKIKLNFDYPDFLGSNSLGKYAFIYKDSKGFYYLGVLDHSNSQSKYKPQILKNNTEFYQLEYKQIKFNTLAGKGYIRDFGVKYSEDENCIINLKTLIKKQYLERYPVLKEIVDFQTDDKKIFDAKVKTILEQAYSINFVNIDKNYILEENNNGNLHFFQIYNKDFSENKKINSMENLHTMYFKALFEKENFNGGACFKLNSQGAEIFFREKSINEKKVKDLKTRNENAIEKKRYTENKVFLHLPITLNFINKGYSKYSFGYINDSVKKYIKENKISIIGIDRGEKNLIYFSMINENLEIIELKSLNSLILKVSDLEEKEVNYFEKLSKKEGNRNKERKDWDEIETIKELKEGYISQIVDNLVKLIVKHNAIVVMEDLNSGFKRGRQKIEKQIYQKFELALAKKLNFTVDKNKKHDELGGIYKAYQLTPQIENFQDIYSQTGIIFYTQAAYTSVTCPNCSFRKNIYQKYENESKFKEFFKKYILEIKFEDNCFIIKYKIDEKIDKKKNKLKKLEFQVNTKNQIRLKFEKSLKGKGGETKEFNITEKFKEIFEKHKLDLGNLKEELLAWNGEIQLYKDFMFYFNLLLQLRNSKENDNGGYISCPSCGFHSGNGFQGFSYNGDANGAYNIARKGRIILDKIKKDEKNLGITNVEWDNYYQK

SEQ ID NO: 65 - protein, 1193 aa, synthetic construct
MKTFENFTNLYSLPRTLRFELKPLYKTKELIDSKQELFPKDKRIDEIYQNIIKPCLNELHSDFIEKSMENKDFQNIPDNILKIYSNEKNIDDFKNIEKDLIKQINGFLKSNKTFFAENYSDLLGKNSIDIIIKVFGEKIYKKDDSGKIFLYNDLLGKSYEELINIYFKGFSTYLSNFNKNRENLYDKKNEAKVGSVSGRTIGENFPRFLQNCINFRDKLEKLNLSIEQKDIFITNNFGKCISQKQIDYYNKIIGQINSKTNEFNQKNGLKGNKKLPKLLLLHKQILGKSENENILNFINNIIQTDFELEQEIKIINKDIFQRIDFIKKSIVSNIEDFELEKIFIKKNRLKDISSLLMDNYSVLEKLLPEFNEEGKIIKENELVNLSKIKKSFENIDLKDLKNIFKKEYFDESKDGFKLFLNGIYNHFSDLENNIKHTHKLVQDKLISGNFSENIQKSEKNINLRDEIFVSSKGLLKAYLDSILALDRFVHMFDYGEQKDFDSNFYNNIEEYSINFSPFKTYNAVRNYLTKKNYSTDKIKLNFDYPDFLRSNSLGKYAFIYKDSKGFYYLGVLDHSNSQSKYKPQILKNNTEFYQLEYKQIKFNTLAGKGYIRDFGVKYSEDENCIINLKTLIKKQYLERYPVLKEIVDFQTDDKKIFDAKVKTILEQAYSINFVNIDKNYILEENNNGNLHFFQIYNKDFSENKKINSMENLHTMYFKALFEKENFNGGACFKLNSQGAEIFFREKSINEKKVKDLKTRNENAIEKKRYTENKVFLHLPITLNFINKGYSKYSFGYINDSVKKYIKENKISIIGIDRGEKNLIYFSMINENLEIIELKSLNSLILKVSDLEEKEVNYFEKLSKKEGNRNKERKDWDEIETIKELKEGYISQIVDNLVKLIVKHNAIVVMEDLNSGFKRGRQKIEKQIYQKFELALAKKLNFTVDKNKKHDELGGIYKAYQLTPQIENFQDIYSQTGIIFYTQAAYTSVTCPNCSFRKNIYQKYENESKFKEFFKKYILEIKFEDNCFIIKYKIDEKIDKKKNKLKKLEFQVNTKNQIRLKFEKSLKGKGGETKEFNITEKFKEIFEKHKLDLGNLKEELLAWNGEIQLYKDFMFYFNLLLQLRNSKENDNGGYISCPSCGFHSGNGFQGFSYNGDANGAYNIARKGRIILDKIKKDEKNLGITNVEWDNYYQK

SEQ ID NO: 66 - protein, 1193 aa, synthetic construct
MKTFENFTNLYSLPRTLRFELKPLYKTKELIDSKQELFPKDKRIDEIYQNIIKPCLNELHSDFIEKSMENKDFQNIPDNILKIYSNEKNIDDFKNIEKDLIKQINGFLKSNKTFFAENYSDLLGKNSIDIIIKVFGEKIYKKDDSGKIFLYNDLLGKSYEELINIYFKGFSTYLSNFNKNRENLYDKKNEAKVGSVSGRTIGENFPRFLQNCINFRDKLEKLNLSIEQKDIFITNNFGKCISQKQIDYYNKIIGQINSKTNEFNQKNGLKGNKKLPKLLLLHKQILGKSENENILNFINNIIQTDFELEQEIKIINKDIFQRIDFIKKSIVSNIEDFELEKIFIKKNRLKDISSLLMDNYSVLEKLLPEFNEEGKIIKENELVNLSKIKKSFENIDLKDLKNIFKKEYFDESKDGFKLFLNGIYNHFSDLENNIKHTHKLVQDKLISGNFSENIQKSEKNINLRDEIFVSSKGLLKAYLDSILALDRFVHMFDYGEQKDFDSNFYNNIEEYSINFSPFKTYNAVRNYLTKKNYSTDKIKLNFDYPDFLGSNSLGRYAFIYKDSKGFYYLGVLDHSNSQSKYKPQILKNNTEFYQLEYKQIKFNTLAGKGYIRDFGVKYSEDENCIINLKTLIKKQYLERYPVLKEIVDFQTDDKKIFDAKVKTILEQAYSINFVNIDKNYILEENNNGNLHFFQIYNKDFSENKKINSMENLHTMYFKALFEKENFNGGACFKLNSQGAEIFFREKSINEKKVKDLKTRNENAIEKKRYTENKVFLHLPITLNFINKGYSKYSFGYINDSVKKYIKENKISIIGIDRGEKNLIYFSMINENLEIIELKSLNSLILKVSDLEEKEVNYFEKLSKKEGNRNKERKDWDEIETIKELKEGYISQIVDNLVKLIVKHNAIVVMEDLNSGFKRGRQKIEKQIYQKFELALAKKLNFTVDKNKKHDELGGIYKAYQLTPQIENFQDIYSQTGIIFYTQAAYTSVTCPNCSFRKNIYQKYENESKFKEFFKKYILEIKFEDNCFIIKYKIDEKIDKKKNKLKKLEFQVNTKNQIRLKFEKSLKGKGGETKEFNITEKFKEIFEKHKLDLGNLKEELLAWNGEIQLYKDFMFYFNLLLQLRNSKENDNGGYISCPSCGFHSGNGFQGFSYNGDANGAYNIARKGRIILDKIKKDEKNLGITNVEWDNYYQK

SEQ ID NO: 67 - protein, 1193 aa, synthetic construct
MKTFENFTNLYSLPRTLRFELKPLYKTKELIDSKQELFPKDKRIDEIYQNIIKPCLNELHSDFIEKSMENKDFQNIPDNILKIYSNEKNIDDFKNIEKDLIKQINGFLKSNKTFFAENYSDLLGKNSIDIIIKVFGEKIYKKDDSGKIFLYNDLLGKSYEELINIYFKGFSTYLSNFNRNRENLYDKKNEAKVGSVSGRTIGENFPRFLQNCINFRDKLEKLNLSIEQKDIFITNNFGKCISQKQIDYYNKIIGQINSKTNEFNQKNGLKGNKKLPKLLLLHKQILGKSENENILNFINNIIQTDFELEQEIKIINKDIFQRIDFIKKSIVSNIEDFELEKIFIKKNRLKDISSLLMDNYSVLEKLLPEFNEEGKIIKENELVNLSKIKKSFENIDLKDLKNIFKKEYFDESKDGFKLFLNGIYNHFSDLENNIKHTHKLVQDKLISGNFSENIQKSEKNINLRDEIFVSSKGLLKAYLDSILALDRFVHMFDYGEQKDFDSNFYNNIEEYSINFSPFKTYNAVRNYLTKKNYSTDKIKLNFDYPDFLRSNSLGKYAFIYKDSKGFYYLGVLDHSNSQSKYKPQILKNNTEFYQLEYKQIKFNTLAGKGYIRDFGVKYSEDENCIINLKTLIKKQYLERYPVLKEIVDFQTDDKKIFDAKVKTILEQAYSINFVNIDKNYILEENNNGNLHFFQIYNKDFSENKKINSMENLHTMYFKALFEKENFNGGACFKLNSQGAEIFFREKSINEKKVKDLKTRNENAIEKKRYTENKVFLHLPITLNFINKGYSKYSFGYINDSVKKYIKENKISIIGIDRGEKNLIYFSMINENLEIIELKSLNSLILKVSDLEEKEVNYFEKLSKKEGNRNKERKDWDEIETIKELKEGYISQIVDNLVKLIVKHNAIVVMEDLNSGFKRGRQKIEKQIYQKFELALAKKLNFTVDKNKKHDELGGIYKAYQLTPQIENFQDIYSQTGIIFYTQAAYTSVTCPNCSFRKNIYQKYENESKFKEFFKKYILEIKFEDNCFIIKYKIDEKIDKKKNKLKKLEFQVNTKNQIRLKFEKSLKGKGGETKEFNITEKFKEIFEKHKLDLGNLKEELLAWNGEIQLYKDFMFYFNLLLQLRNSKENDNGGYISCPSCGFHSGNGFQGFSYNGDANGAYNIARKGRIILDKIKKDEKNLGITNVEWDNYYQK

SEQ ID NO: 68 - protein, 1193 aa, synthetic construct
MKTFENFTNLYSLPRTLRFELKPLYKTKELIDSKQELFPKDKRIDEIYQNIIKPCLNELHSDFIEKSMENKDFQNIPDNILKIYSNEKNIDDFKNIEKDLIKQINGFLKSNKTFFAENYSDLLGKNSIDIIIKVFGEKIYKKDDSGKIFLYNDLLGKSYEELINIYFKGFSTYLSNFNRNRENLYDKKNEAKVGSVSGRTIGENFPRFLQNCINFRDKLEKLNLSIEQKDIFITNNFGKCISQKQIDYYNKIIGQINSKTNEFNQKNGLKGNKKLPKLLLLHKQILGKSENENILNFINNIIQTDFELEQEIKIINKDIFQRIDFIKKSIVSNIEDFELEKIFIKKNRLKDISSLLMDNYSVLEKLLPEFNEEGKIIKENELVNLSKIKKSFENIDLKDLKNIFKKEYFDESKDGFKLFLNGIYNHFSDLENNIKHTHKLVQDKLISGNFSENIQKSEKNINLRDEIFVSSKGLLKAYLDSILALDRFVHMFDYGEQKDFDSNFYNNIEEYSINFSPFKTYNAVRNYLTKKNYSTDKIKLNFDYPDFLGSNSLGRYAFIYKDSKGFYYLGVLDHSNSQSKYKPQILKNNTEFYQLEYKQIKFNTLAGKGYIRDFGVKYSEDENCIINLKTLIKKQYLERYPVLKEIVDFQTDDKKIFDAKVKTILEQAYSINFVNIDKNYILEENNNGNLHFFQIYNKDFSENKKINSMENLHTMYFKALFEKENFNGGACFKLNSQGAEIFFREKSINEKKVKDLKTRNENAIEKKRYTENKVFLHLPITLNFINKGYSKYSFGYINDSVKKYIKENKISIIGIDRGEKNLIYFSMINENLEIIELKSLNSLILKVSDLEEKEVNYFEKLSKKEGNRNKERKDWDEIETIKELKEGYISQIVDNLVKLIVKHNAIVVMEDLNSGFKRGRQKIEKQIYQKFELALAKKLNFTVDKNKKHDELGGIYKAYQLTPQIENFQDIYSQTGIIFYTQAAYTSVTCPNCSFRKNIYQKYENESKFKEFFKKYILEIKFEDNCFIIKYKIDEKIDKKKNKLKKLEFQVNTKNQIRLKFEKSLKGKGGETKEFNITEKFKEIFEKHKLDLGNLKEELLAWNGEIQLYKDFMFYFNLLLQLRNSKENDNGGYISCPSCGFHSGNGFQGFSYNGDANGAYNIARKGRIILDKIKKDEKNLGITNVEWDNYYQK

SEQ ID NO: 69 - protein, 1193 aa, synthetic construct
MKTFENFTNLYSLPRTLRFELKPLYKTKELIDSKQELFPKDKRIDEIYQNIIKPCLNELHSDFIEKSMENKDFQNIPDNILKIYSNEKNIDDFKNIEKDLIKQINGFLKSNKTFFAENYSDLLGKNSIDIIIKVFGEKIYKKDDSGKIFLYNDLLGKSYEELINIYFKGFSTYLSNFNKNRENLYDKKNEAKVGSVSGRTIGENFPRFLQNCINFRDKLEKLNLSIEQKDIFITNNFGKCISQKQIDYYNKIIGQINSKTNEFNQKNGLKGNKKLPKLLLLHKQILGKSENENILNFINNIIQTDFELEQEIKIINKDIFQRIDFIKKSIVSNIEDFELEKIFIKKNRLKDISSLLMDNYSVLEKLLPEFNEEGKIIKENELVNLSKIKKSFENIDLKDLKNIFKKEYFDESKDGFKLFLNGIYNHFSDLENNIKHTHKLVQDKLISGNFSENIQKSEKNINLRDEIFVSSKGLLKAYLDSILALDRFVHMFDYGEQKDFDSNFYNNIEEYSINFSPFKTYNAVRNYLTKKNYSTDKIKLNFDYPDFLRSNSLGRYAFIYKDSKGFYYLGVLDHSNSQSKYKPQILKNNTEFYQLEYKQIKFNTLAGKGYIRDFGVKYSEDENCIINLKTLIKKQYLERYPVLKEIVDFQTDDKKIFDAKVKTILEQAYSINFVNIDKNYILEENNNGNLHFFQIYNKDFSENKKINSMENLHTMYFKALFEKENFNGGACFKLNSQGAEIFFREKSINEKKVKDLKTRNENAIEKKRYTENKVFLHLPITLNFINKGYSKYSFGYINDSVKKYIKENKISIIGIDRGEKNLIYFSMINENLEIIELKSLNSLILKVSDLEEKEVNYFEKLSKKEGNRNKERKDWDEIETIKELKEGYISQIVDNLVKLIVKHNAIVVMEDLNSGFKRGRQKIEKQIYQKFELALAKKLNFTVDKNKKHDELGGIYKAYQLTPQIENFQDIYSQTGIIFYTQAAYTSVTCPNCSFRKNIYQKYENESKFKEFFKKYILEIKFEDNCFIIKYKIDEKIDKKKNKLKKLEFQVNTKNQIRLKFEKSLKGKGGETKEFNITEKFKEIFEKHKLDLGNLKEELLAWNGEIQLYKDFMFYFNLLLQLRNSKENDNGGYISCPSCGFHSGNGFQGFSYNGDANGAYNIARKGRIILDKIKKDEKNLGITNVEWDNYYQK

SEQ ID NO: 70 - protein, 1193 aa, synthetic construct
MKTFENFTNLYSLPRTLRFELKPLYKTKELIDSKQELFPKDKRIDEIYQNIIKPCLNELHSDFIEKSMENKDFQNIPDNILKIYSNEKNIDDFKNIEKDLIKQINGFLKSNKTFFAENYSDLLGKNSIDIIIKVFGEKIYKKDDSGKIFLYNDLLGKSYEELINIYFKGFSTYLSNFNRNRENLYDKKNEAKVGSVSGRTIGENFPRFLQNCINFRDKLEKLNLSIEQKDIFITNNFGKCISQKQIDYYNKIIGQINSKTNEFNQKNGLKGNKKLPKLLLLHKQILGKSENENILNFINNIIQTDFELEQEIKIINKDIFQRIDFIKKSIVSNIEDFELEKIFIKKNRLKDISSLLMDNYSVLEKLLPEFNEEGKIIKENELVNLSKIKKSFENIDLKDLKNIFKKEYFDESKDGFKLFLNGIYNHFSDLENNIKHTHKLVQDKLISGNFSENIQKSEKNINLRDEIFVSSKGLLKAYLDSILALDRFVHMFDYGEQKDFDSNFYNNIEEYSINFSPFKTYNAVRNYLTKKNYSTDKIKLNFDYPDFLRSNSLGRYAFIYKDSKGFYYLGVLDHSNSQSKYKPQILKNNTEFYQLEYKQIKFNTLAGKGYIRDFGVKYSEDENCIINLKTLIKKQYLERYPVLKEIVDFQTDDKKIFDAKVKTILEQAYSINFVNIDKNYILEENNNGNLHFFQIYNKDFSENKKINSMENLHTMYFKALFEKENFNGGACFKLNSQGAEIFFREKSINEKKVKDLKTRNENAIEKKRYTENKVFLHLPITLNFINKGYSKYSFGYINDSVKKYIKENKISIIGIDRGEKNLIYFSMINENLEIIELKSLNSLILKVSDLEEKEVNYFEKLSKKEGNRNKERKDWDEIETIKELKEGYISQIVDNLVKLIVKHNAIVVMEDLNSGFKRGRQKIEKQIYQKFELALAKKLNFTVDKNKKHDELGGIYKAYQLTPQIENFQDIYSQTGIIFYTQAAYTSVTCPNCSFRKNIYQKYENESKFKEFFKKYILEIKFEDNCFIIKYKIDEKIDKKKNKLKKLEFQVNTKNQIRLKFEKSLKGKGGETKEFNITEKFKEIFEKHKLDLGNLKEELLAWNGEIQLYKDFMFYFNLLLQLRNSKENDNGGYISCPSCGFHSGNGFQGFSYNGDANGAYNIARKGRIILDKIKKDEKNLGITNVEWDNYYQK

SEQ ID NO: 71 - protein, 1193 aa, synthetic construct
MKTFENFTNLYSLPRTLRFELKPLYKTKELIDSKQELFPKDKRIDEIYQNIIKPCLNELHSDFIEKSMENKDFQNIPDNILKIYSNEKNIDDFKNIEKDLIKQINGFLKSNKTFFAENYSDLLGKNSIDIIIKVFGEKIYKKDDSGKIFLYNDLLGKSYEELINIYFKGFSTYLSNFNRNRENLYDKKNEAKVGSVSGRTIGENFPRFLQNCINFRDKLEKLNLSIEQKDIFITNNFGKCISQKQIDYYNKIIGQINSKTAEFNQKNGLKGNKKLPKLLLLHKQILGKSENENILNFINNIIQTDFELEQEIKIINKDIFQRIDFIKKSIVSNIEDFELEKIFIKKNRLKDISSLLMDNYSVLEKLLPEFNEEGKIIKENELVNLSKIKKSFENIDLKDLKNIFKKEYFDESKDGFKLFLNGIYNHFSDLENNIKHTHKLVQDKLISGNFSENIQKSEKNINLRDEIFVSSKGLLKAYLDSILALDRFVHMFDYGEQKDFDSNFYNNIEEYSINFSPFKTYNAVRNYLTKKNYSTDKIKLNFDYPDFLGSNSLGRYAFIYKDSKGFYYLGVLDHSNSQSKYKPQILKNNTEFYQLEYKQIKFNTLAGKGYIRDFGVKYSEDENCIINLKTLIKKQYLERYPVLKEIVDFQTDDKKIFDAKVKTILEQAYSINFVNIDKNYILEENNNGNLHFFQIYNKDFSENKKINSMENLHTMYFKALFEKENFNGGACFKLNSQGAEIFFREKSINEKKVKDLKTRNENAIEKKRYTENKVFLHLPITLNFINKGYSKYSFGYINDSVKKYIKENKISIIGIDRGEKNLIYFSMINENLEIIELKSLNSLILKVSDLEEKEVNYFEKLSKKEGNRNKERKDWDEIETIKELKEGYISQIVDNLVKLIVKHNAIVVMEDLNSGFKRGRQKIEKQIYQKFELALAKKLNFTVDKNKKHDELGGIYKAYQLTPQIENFQDIYSQTGIIFYTQAAYTSVTCPNCSFRKNIYQKYENESKFKEFFKKYILEIKFEDNCFIIKYKIDEKIDKKKNKLKKLEFQVNTKNQIRLKFEKSLKGKGGETKEFNITEKFKEIFEKHKLDLGNLKEELLAWNGEIQLYKDFMFYFNLLLQLRNSKENDNGGYISCPSCGFHSGNGFQGFSYNGDANGAYNIARKGRIILDKIKKDEKNLGITNVEWDNYYQK

SEQ ID NO: 72 - protein, 1193 aa, synthetic construct
MKTFENFTNLYSLPRTLRFELKPLYKTKELIDSKQELFPKDKRIDEIYQNIIKPCLNELHSDFIEKSMENKDFQNIPDNILKIYSNEKNIDDFKNIEKDLIKQINGFLKSNKTFFAENYSDLLGKNSIDIIIKVFGEKIYKKDDSGKIFLYNDLLGKSYEELINIYFKGFSTYLSNFNKNRENLYDKKNEAKVGSVSGRTIGENFPRFLQNCINFRDKLEKLNLSIEQKDIFITNNFGKCISQKQIDYYNKIIGQINSKTNEFNQKNGLKGNKKLPKLLLLHKQILGKSENENILNFINNIIQTDFELEQEIKIINKDIFQRIDFIKKSIVSNIEDFELEKIFIKKNRLKDISSLLMDNYSVLEKLLPEFNEEGKIIKENELVNLSKIKKSFENIDLKDLKNIFKKEYFDESKDGFKLFLNGIYNHFSDLENNIKHTHKLVQDKLISGNFSENIQKSEKNINLRDEIFVSSKGLLKAYLDSILALDRFVHMFDYGEQKDFDSNFYNNIEEYSINFSPFKTYNAVRNYLTKKNYSTDKIKLNFDRPDFLGSNSLGKYAFIYKDSKGFYYLGVLDHSNSQSKYKPQILKNNTEFYQLEYKQIKFNTLAGKGYIRDFGVKYSEDENCIINLKTLIKKQYLERYPVLKEIVDFQTDDKKIFDAKVKTILEQAYSINFVNIDKNYILEENNNGNLHFFQIYNKDFSENKKINSMENLHTMYFKALFEKENFNGGACFKLNSQGAEIFFREKSINEKKVKDLKTRNENAIEKKRYTENKVFLHLPITLNFINKGYSKYSFGYINDSVKKYIKENKISIIGIDRGEKNLIYFSMINENLEIIELKSLNSLILKVSDLEEKEVNYFEKLSKKEGNRNKERKDWDEIETIKELKEGYISQIVDNLVKLIVKHNAIVVMEDLNSGFKRGRQKIEKQIYQKFELALAKKLNFTVDKNKKHDELGGIYKAYQLTPQIENFQDIYSQTGIIFYTQAAYTSVTCPNCSFRKNIYQKYENESKFKEFFKKYILEIKFEDNCFIIKYKIDEKIDKKKNKLKKLEFQVNTKNQIRLKFEKSLKGKGGETKEFNITEKFKEIFEKHKLDLGNLKEELLAWNGEIQLYKDFMFYFNLLLQLRNSKENDNGGYISCPSCGFHSGNGFQGFSYNGDANGAYNIARKGRIILDKIKKDEKNLGITNVEWDNYYQK

SEQ ID NO: 73 - protein, 1221 aa, synthetic construct
MEKFYDKFNNLFSVSKTLRFELEPIGKTKEFFEKYILKEDERKSDLFKKVKKYCDEYHKLFISECLKNFDDKDFENLLKEYFALVNLSNPSEKETKRITDILKVLRAKISDRFTKNPKYKGLFGKELINDYLKDYYKNNKNILDEITPFKDFTSYFTGFNRNRKNMYSKEEKHTAIAYRLIDENLSTYIKNIKAFNIILENIPDIKIQIKENLNLDCDEFFSNIENYTNVLTQEQIETYNLAISGKSQENSKIKGINEIVNLYKQKNKIKIPKLKELYKQLLSDTTSASFKFDIIEYDYQIVEMINSYYETFKKNFIDDNGCQSALSNIQNYDIKNIYINNDVSLTSLSQNIYNDWNYINSLLGNEYDQNYKGKAKFGTEKYSEKKEETLKKIKEISLLKIEELIKNYDKENNNSEKIFEYFKNAIEENLKKIDIEYKNTKTILQKEYDKTSTDLLKDEQSIEKIKNFLDSIKDLQILIKYLIPKNNTLETDLEFYNALNYDILSEIIPVYNKTRNYLTKKTYSLEKFKLNFDCPTFLNGWDLNKEEANLGTLFRKDGNYYIGIMNTNSRKSFIEYNQAEFKNEKKYQKIEYKLLPNPNQMLPKVFFSESRIKEFNPSEELLSKYERGYHKKGADFDLNFCHQLIDFFKSSINKHEDWSKFNFKFSETNKYNDISEFYKEVELQGYKISFTDISEEYINKLVEEGKLYLFRIYNKDFSSYSKGTPNLHTLYWKALFDEDNLKNIIYKLNGGAEIFYRKKSIEAKITHPKNQPIDNKNPNNPKKQSIFEYDLIKDKRFTLDKFQFHVPITLNFKAKDTHKLNDLVNNKIKNTEDINIIGIDRGERHLLYACVINKNGEILEQYSLNTIGQTNYHYLLSKKEDDRLKQRQAWGTIQNIKELKEGYMSQVIHKLTKLMVQYNAVIVLEDLNSGFKNSRKKVEKSVYDKFETALINKLSYLVDKSIQNKFEQGGIMNAYQLANADIKSAKQNGVIFYIPAWCTSKIDPTTGFVNLFDLKKIDKEFVKKFNSIKFNNTENYFEFDFDYSNFSDKSSGDRTNWTLCSFGNRIRTFKNPKKNNNYDSQTVELTQEFKKLFDCYNLDCSNLRDEILNKSDSKFFNATPEKNNFYGFALLFKLLLQLRNSITGSEEDYILSPIKNKKGLFYDSRTANGVLPENADANGAYNIARKGLLILNRIKNSEENTKTDYTIKNADWLTYVQQQDR

SEQ ID NO: 74 - protein, 1221 aa, synthetic construct
MEKFYDKFNNLFSVSKTLRFELEPIGKTKEFFEKYILKEDERKSDLFKKVKKYCDEYHKLFISECLKNFDDKDFENLLKEYFALVNLSNPSEKETKRITDILKVLRAKISDRFTKNPKYKGLFGKELINDYLKDYYKNNKNILDEITPFKDFTSYFTGFNTNRKNMYSKEEKHTAIAYRLIDENLSTYIKNIKAFNIILENIPDIKIQIKENLNLDCDEFFSNIENYTNVLTQEQIETYNLAISGKSQENSKIKGINEIVNLYKQKNKIKIPKLKELYKQLLSDTTSASFKFDIIEYDYQIVEMINSYYETFKKNFIDDNGCQSALSNIQNYDIKNIYINNDVSLTSLSQNIYNDWNYINSLLGNEYDQNYKGKAKFGTEKYSEKKEETLKKIKEISLLKIEELIKNYDKENNNSEKIFEYFKNAIEENLKKIDIEYKNTKTILQKEYDKTSTDLLKDEQSIEKIKNFLDSIKDLQILIKYLIPKNNTLETDLEFYNALNYDILSEIIPVYNKTRNYLTKKTYSLEKFKLNFDCPTFLRGWDLNKEEANLGTLFRKDGNYYIGIMNTNSRKSFIEYNQAEFKNEKKYQKIEYKLLPNPNQMLPKVFFSESRIKEFNPSEELLSKYERGYHKKGADFDLNFCHQLIDFFKSSINKHEDWSKFNFKFSETNKYNDISEFYKEVELQGYKISFTDISEEYINKLVEEGKLYLFRIYNKDFSSYSKGTPNLHTLYWKALFDEDNLKNIIYKLNGGAEIFYRKKSIEAKITHPKNQPIDNKNPNNPKKQSIFEYDLIKDKRFTLDKFQFHVPITLNFKAKDTHKLNDLVNNKIKNTEDINIIGIDRGERHLLYACVINKNGEILEQYSLNTIGQTNYHYLLSKKEDDRLKQRQAWGTIQNIKELKEGYMSQVIHKLTKLMVQYNAVIVLEDLNSGFKNSRKKVEKSVYDKFETALINKLSYLVDKSIQNKFEQGGIMNAYQLANADIKSAKQNGVIFYIPAWCTSKIDPTTGFVNLFDLKKIDKEFVKKFNSIKFNNTENYFEFDFDYSNFSDKSSGDRTNWTLCSFGNRIRTFKNPKKNNNYDSQTVELTQEFKKLFDCYNLDCSNLRDEILNKSDSKFFNATPEKNNFYGFALLFKLLLQLRNSITGSEEDYILSPIKNKKGLFYDSRTANGVLPENADANGAYNIARKGLLILNRIKNSEENTKTDYTIKNADWLTYVQQQDR

SEQ ID NO: 75 - protein, 1221 aa, synthetic construct
MEKFYDKFNNLFSVSKTLRFELEPIGKTKEFFEKYILKEDERKSDLFKKVKKYCDEYHKLFISECLKNFDDKDFENLLKEYFALVNLSNPSEKETKRITDILKVLRAKISDRFTKNPKYKGLFGKELINDYLKDYYKNNKNILDEITPFKDFTSYFTGFNTNRKNMYSKEEKHTAIAYRLIDENLSTYIKNIKAFNIILENIPDIKIQIKENLNLDCDEFFSNIENYTNVLTQEQIETYNLAISGKSQENSKIKGINEIVNLYKQKNKIKIPKLKELYKQLLSDTTSASFKFDIIEYDYQIVEMINSYYETFKKNFIDDNGCQSALSNIQNYDIKNIYINNDVSLTSLSQNIYNDWNYINSLLGNEYDQNYKGKAKFGTEKYSEKKEETLKKIKEISLLKIEELIKNYDKENNNSEKIFEYFKNAIEENLKKIDIEYKNTKTILQKEYDKTSTDLLKDEQSIEKIKNFLDSIKDLQILIKYLIPKNNTLETDLEFYNALNYDILSEIIPVYNKTRNYLTKKTYSLEKFKLNFDCPTFLNGWDLNREEANLGTLFRKDGNYYIGIMNTNSRKSFIEYNQAEFKNEKKYQKIEYKLLPNPNQMLPKVFFSESRIKEFNPSEELLSKYERGYHKKGADFDLNFCHQLIDFFKSSINKHEDWSKFNFKFSETNKYNDISEFYKEVELQGYKISFTDISEEYINKLVEEGKLYLFRIYNKDFSSYSKGTPNLHTLYWKALFDEDNLKNIIYKLNGGAEIFYRKKSIEAKITHPKNQPIDNKNPNNPKKQSIFEYDLIKDKRFTLDKFQFHVPITLNFKAKDTHKLNDLVNNKIKNTEDINIIGIDRGERHLLYACVINKNGEILEQYSLNTIGQTNYHYLLSKKEDDRLKQRQAWGTIQNIKELKEGYMSQVIHKLTKLMVQYNAVIVLEDLNSGFKNSRKKVEKSVYDKFETALINKLSYLVDKSIQNKFEQGGIMNAYQLANADIKSAKQNGVIFYIPAWCTSKIDPTTGFVNLFDLKKIDKEFVKKFNSIKFNNTENYFEFDFDYSNFSDKSSGDRTNWTLCSFGNRIRTFKNPKKNNNYDSQTVELTQEFKKLFDCYNLDCSNLRDEILNKSDSKFFNATPEKNNFYGFALLFKLLLQLRNSITGSEEDYILSPIKNKKGLFYDSRTANGVLPENADANGAYNIARKGLLILNRIKNSEENTKTDYTIKNADWLTYVQQQDR

SEQ ID NO: 76 - protein, 1221 aa, synthetic construct
MEKFYDKFNNLFSVSKTLRFELEPIGKTKEFFEKYILKEDERKSDLFKKVKKYCDEYHKLFISECLKNFDDKDFENLLKEYFALVNLSNPSEKETKRITDILKVLRAKISDRFTKNPKYKGLFGKELINDYLKDYYKNNKNILDEITPFKDFTSYFTGFNRNRKNMYSKEEKHTAIAYRLIDENLSTYIKNIKAFNIILENIPDIKIQIKENLNLDCDEFFSNIENYTNVLTQEQIETYNLAISGKSQENSKIKGINEIVNLYKQKNKIKIPKLKELYKQLLSDTTSASFKFDIIEYDYQIVEMINSYYETFKKNFIDDNGCQSALSNIQNYDIKNIYINNDVSLTSLSQNIYNDWNYINSLLGNEYDQNYKGKAKFGTEKYSEKKEETLKKIKEISLLKIEELIKNYDKENNNSEKIFEYFKNAIEENLKKIDIEYKNTKTILQKEYDKTSTDLLKDEQSIEKIKNFLDSIKDLQILIKYLIPKNNTLETDLEFYNALNYDILSEIIPVYNKTRNYLTKKTYSLEKFKLNFDCPTFLRGWDLNKEEANLGTLFRKDGNYYIGIMNTNSRKSFIEYNQAEFKNEKKYQKIEYKLLPNPNQMLPKVFFSESRIKEFNPSEELLSKYERGYHKKGADFDLNFCHQLIDFFKSSINKHEDWSKFNFKFSETNKYNDISEFYKEVELQGYKISFTDISEEYINKLVEEGKLYLFRIYNKDFSSYSKGTPNLHTLYWKALFDEDNLKNIIYKLNGGAEIFYRKKSIEAKITHPKNQPIDNKNPNNPKKQSIFEYDLIKDKRFTLDKFQFHVPITLNFKAKDTHKLNDLVNNKIKNTEDINIIGIDRGERHLLYACVINKNGEILEQYSLNTIGQTNYHYLLSKKEDDRLKQRQAWGTIQNIKELKEGYMSQVIHKLTKLMVQYNAVIVLEDLNSGFKNSRKKVEKSVYDKFETALINKLSYLVDKSIQNKFEQGGIMNAYQLANADIKSAKQNGVIFYIPAWCTSKIDPTTGFVNLFDLKKIDKEFVKKFNSIKFNNTENYFEFDFDYSNFSDKSSGDRTNWTLCSFGNRIRTFKNPKKNNNYDSQTVELTQEFKKLFDCYNLDCSNLRDEILNKSDSKFFNATPEKNNFYGFALLFKLLLQLRNSITGSEEDYILSPIKNKKGLFYDSRTANGVLPENADANGAYNIARKGLLILNRIKNSEENTKTDYTIKNADWLTYVQQQDR

SEQ ID NO: 77 - protein, 1221 aa, synthetic construct
MEKFYDKFNNLFSVSKTLRFELEPIGKTKEFFEKYILKEDERKSDLFKKVKKYCDEYHKLFISECLKNFDDKDFENLLKEYFALVNLSNPSEKETKRITDILKVLRAKISDRFTKNPKYKGLFGKELINDYLKDYYKNNKNILDEITPFKDFTSYFTGFNRNRKNMYSKEEKHTAIAYRLIDENLSTYIKNIKAFNIILENIPDIKIQIKENLNLDCDEFFSNIENYTNVLTQEQIETYNLAISGKSQENSKIKGINEIVNLYKQKNKIKIPKLKELYKQLLSDTTSASFKFDIIEYDYQIVEMINSYYETFKKNFIDDNGCQSALSNIQNYDIKNIYINNDVSLTSLSQNIYNDWNYINSLLGNEYDQNYKGKAKFGTEKYSEKKEETLKKIKEISLLKIEELIKNYDKENNNSEKIFEYFKNAIEENLKKIDIEYKNTKTILQKEYDKTSTDLLKDEQSIEKIKNFLDSIKDLQILIKYLIPKNNTLETDLEFYNALNYDILSEIIPVYNKTRNYLTKKTYSLEKFKLNFDCPTFLNGWDLNREEANLGTLFRKDGNYYIGIMNTNSRKSFIEYNQAEFKNEKKYQKIEYKLLPNPNQMLPKVFFSESRIKEFNPSEELLSKYERGYHKKGADFDLNFCHQLIDFFKSSINKHEDWSKFNFKFSETNKYNDISEFYKEVELQGYKISFTDISEEYINKLVEEGKLYLFRIYNKDFSSYSKGTPNLHTLYWKALFDEDNLKNIIYKLNGGAEIFYRKKSIEAKITHPKNQPIDNKNPNNPKKQSIFEYDLIKDKRFTLDKFQFHVPITLNFKAKDTHKLNDLVNNKIKNTEDINIIGIDRGERHLLYACVINKNGEILEQYSLNTIGQTNYHYLLSKKEDDRLKQRQAWGTIQNIKELKEGYMSQVIHKLTKLMVQYNAVIVLEDLNSGFKNSRKKVEKSVYDKFETALINKLSYLVDKSIQNKFEQGGIMNAYQLANADIKSAKQNGVIFYIPAWCTSKIDPTTGFVNLFDLKKIDKEFVKKFNSIKFNNTENYFEFDFDYSNFSDKSSGDRTNWTLCSFGNRIRTFKNPKKNNNYDSQTVELTQEFKKLFDCYNLDCSNLRDEILNKSDSKFFNATPEKNNFYGFALLFKLLLQLRNSITGSEEDYILSPIKNKKGLFYDSRTANGVLPENADANGAYNIARKGLLILNRIKNSEENTKTDYTIKNADWLTYVQQQDR

SEQ ID NO: 78 - protein, 1221 aa, synthetic construct
MEKFYDKFNNLFSVSKTLRFELEPIGKTKEFFEKYILKEDERKSDLFKKVKKYCDEYHKLFISECLKNFDDKDFENLLKEYFALVNLSNPSEKETKRITDILKVLRAKISDRFTKNPKYKGLFGKELINDYLKDYYKNNKNILDEITPFKDFTSYFTGFNTNRKNMYSKEEKHTAIAYRLIDENLSTYIKNIKAFNIILENIPDIKIQIKENLNLDCDEFFSNIENYTNVLTQEQIETYNLAISGKSQENSKIKGINEIVNLYKQKNKIKIPKLKELYKQLLSDTTSASFKFDIIEYDYQIVEMINSYYETFKKNFIDDNGCQSALSNIQNYDIKNIYINNDVSLTSLSQNIYNDWNYINSLLGNEYDQNYKGKAKFGTEKYSEKKEETLKKIKEISLLKIEELIKNYDKENNNSEKIFEYFKNAIEENLKKIDIEYKNTKTILQKEYDKTSTDLLKDEQSIEKIKNFLDSIKDLQILIKYLIPKNNTLETDLEFYNALNYDILSEIIPVYNKTRNYLTKKTYSLEKFKLNFDCPTFLRGWDLNREEANLGTLFRKDGNYYIGIMNTNSRKSFIEYNQAEFKNEKKYQKIEYKLLPNPNQMLPKVFFSESRIKEFNPSEELLSKYERGYHKKGADFDLNFCHQLIDFFKSSINKHEDWSKFNFKFSETNKYNDISEFYKEVELQGYKISFTDISEEYINKLVEEGKLYLFRIYNKDFSSYSKGTPNLHTLYWKALFDEDNLKNIIYKLNGGAEIFYRKKSIEAKITHPKNQPIDNKNPNNPKKQSIFEYDLIKDKRFTLDKFQFHVPITLNFKAKDTHKLNDLVNNKIKNTEDINIIGIDRGERHLLYACVINKNGEILEQYSLNTIGQTNYHYLLSKKEDDRLKQRQAWGTIQNIKELKEGYMSQVIHKLTKLMVQYNAVIVLEDLNSGFKNSRKKVEKSVYDKFETALINKLSYLVDKSIQNKFEQGGIMNAYQLANADIKSAKQNGVIFYIPAWCTSKIDPTTGFVNLFDLKKIDKEFVKKFNSIKFNNTENYFEFDFDYSNFSDKSSGDRTNWTLCSFGNRIRTFKNPKKNNNYDSQTVELTQEFKKLFDCYNLDCSNLRDEILNKSDSKFFNATPEKNNFYGFALLFKLLLQLRNSITGSEEDYILSPIKNKKGLFYDSRTANGVLPENADANGAYNIARKGLLILNRIKNSEENTKTDYTIKNADWLTYVQQQDR

SEQ ID NO: 79 - protein, 1221 aa, synthetic construct
MEKFYDKFNNLFSVSKTLRFELEPIGKTKEFFEKYILKEDERKSDLFKKVKKYCDEYHKLFISECLKNFDDKDFENLLKEYFALVNLSNPSEKETKRITDILKVLRAKISDRFTKNPKYKGLFGKELINDYLKDYYKNNKNILDEITPFKDFTSYFTGFNRNRKNMYSKEEKHTAIAYRLIDENLSTYIKNIKAFNIILENIPDIKIQIKENLNLDCDEFFSNIENYTNVLTQEQIETYNLAISGKSQENSKIKGINEIVNLYKQKNKIKIPKLKELYKQLLSDTTSASFKFDIIEYDYQIVEMINSYYETFKKNFIDDNGCQSALSNIQNYDIKNIYINNDVSLTSLSQNIYNDWNYINSLLGNEYDQNYKGKAKFGTEKYSEKKEETLKKIKEISLLKIEELIKNYDKENNNSEKIFEYFKNAIEENLKKIDIEYKNTKTILQKEYDKTSTDLLKDEQSIEKIKNFLDSIKDLQILIKYLIPKNNTLETDLEFYNALNYDILSEIIPVYNKTRNYLTKKTYSLEKFKLNFDCPTFLRGWDLNREEANLGTLFRKDGNYYIGIMNTNSRKSFIEYNQAEFKNEKKYQKIEYKLLPNPNQMLPKVFFSESRIKEFNPSEELLSKYERGYHKKGADFDLNFCHQLIDFFKSSINKHEDWSKFNFKFSETNKYNDISEFYKEVELQGYKISFTDISEEYINKLVEEGKLYLFRIYNKDFSSYSKGTPNLHTLYWKALFDEDNLKNIIYKLNGGAEIFYRKKSIEAKITHPKNQPIDNKNPNNPKKQSIFEYDLIKDKRFTLDKFQFHVPITLNFKAKDTHKLNDLVNNKIKNTEDINIIGIDRGERHLLYACVINKNGEILEQYSLNTIGQTNYHYLLSKKEDDRLKQRQAWGTIQNIKELKEGYMSQVIHKLTKLMVQYNAVIVLEDLNSGFKNSRKKVEKSVYDKFETALINKLSYLVDKSIQNKFEQGGIMNAYQLANADIKSAKQNGVIFYIPAWCTSKIDPTTGFVNLFDLKKIDKEFVKKFNSIKFNNTENYFEFDFDYSNFSDKSSGDRTNWTLCSFGNRIRTFKNPKKNNNYDSQTVELTQEFKKLFDCYNLDCSNLRDEILNKSDSKFFNATPEKNNFYGFALLFKLLLQLRNSITGSEEDYILSPIKNKKGLFYDSRTANGVLPENADANGAYNIARKGLLILNRIKNSEENTKTDYTIKNADWLTYVQQQDR

SEQ ID NO: 80 - protein, 1221 aa, synthetic construct
MEKFYDKFNNLFSVSKTLRFELEPIGKTKEFFEKYILKEDERKSDLFKKVKKYCDEYHKLFISECLKNFDDKDFENLLKEYFALVNLSNPSEKETKRITDILKVLRAKISDRFTKNPKYKGLFGKELINDYLKDYYKNNKNILDEITPFKDFTSYFTGFNRNRKNMYSKEEKHTAIAYRLIDENLSTYIKNIKAFNIILENIPDIKIQIKENLNLDCDEFFSNIENYTNVLTQEQIETYNLAISGKSQENSKIKGINEIVALYKQKNKIKIPKLKELYKQLLSDTTSASFKFDIIEYDYQIVEMINSYYETFKKNFIDDNGCQSALSNIQNYDIKNIYINNDVSLTSLSQNIYNDWNYINSLLGNEYDQNYKGKAKFGTEKYSEKKEETLKKIKEISLLKIEELIKNYDKENNNSEKIFEYFKNAIEENLKKIDIEYKNTKTILQKEYDKTSTDLLKDEQSIEKIKNFLDSIKDLQILIKYLIPKNNTLETDLEFYNALNYDILSEIIPVYNKTRNYLTKKTYSLEKFKLNFDCPTFLNGWDLNREEANLGTLFRKDGNYYIGIMNTNSRKSFIEYNQAEFKNEKKYQKIEYKLLPNPNQMLPKVFFSESRIKEFNPSEELLSKYERGYHKKGADFDLNFCHQLIDFFKSSINKHEDWSKFNFKFSETNKYNDISEFYKEVELQGYKISFTDISEEYINKLVEEGKLYLFRIYNKDFSSYSKGTPNLHTLYWKALFDEDNLKNIIYKLNGGAEIFYRKKSIEAKITHPKNQPIDNKNPNNPKKQSIFEYDLIKDKRFTLDKFQFHVPITLNFKAKDTHKLNDLVNNKIKNTEDINIIGIDRGERHLLYACVINKNGEILEQYSLNTIGQTNYHYLLSKKEDDRLKQRQAWGTIQNIKELKEGYMSQVIHKLTKLMVQYNAVIVLEDLNSGFKNSRKKVEKSVYDKFETALINKLSYLVDKSIQNKFEQGGIMNAYQLANADIKSAKQNGVIFYIPAWCTSKIDPTTGFVNLFDLKKIDKEFVKKFNSIKFNNTENYFEFDFDYSNFSDKSSGDRTNWTLCSFGNRIRTFKNPKKNNNYDSQTVELTQEFKKLFDCYNLDCSNLRDEILNKSDSKFFNATPEKNNFYGFALLFKLLLQLRNSITGSEEDYILSPIKNKKGLFYDSRTANGVLPENADANGAYNIARKGLLILNRIKNSEENTKTDYTIKNADWLTYVQQQDR

SEQ ID NO: 81 - protein, 1221 aa, synthetic construct
MEKFYDKFNNLFSVSKTLRFELEPIGKTKEFFEKYILKEDERKSDLFKKVKKYCDEYHKLFISECLKNFDDKDFENLLKEYFALVNLSNPSEKETKRITDILKVLRAKISDRFTKNPKYKGLFGKELINDYLKDYYKNNKNILDEITPFKDFTSYFTGFNTNRKNMYSKEEKHTAIAYRLIDENLSTYIKNIKAFNIILENIPDIKIQIKENLNLDCDEFFSNIENYTNVLTQEQIETYNLAISGKSQENSKIKGINEIVNLYKQKNKIKIPKLKELYKQLLSDTTSASFKFDIIEYDYQIVEMINSYYETFKKNFIDDNGCQSALSNIQNYDIKNIYINNDVSLTSLSQNIYNDWNYINSLLGNEYDQNYKGKAKFGTEKYSEKKEETLKKIKEISLLKIEELIKNYDKENNNSEKIFEYFKNAIEENLKKIDIEYKNTKTILQKEYDKTSTDLLKDEQSIEKIKNFLDSIKDLQILIKYLIPKNNTLETDLEFYNALNYDILSEIIPVYNKTRNYLTKKTYSLEKFKLNFDRPTFLNGWDLNKEEANLGTLFRKDGNYYIGIMNTNSRKSFIEYNQAEFKNEKKYQKIEYKLLPNPNQMLPKVFFSESRIKEFNPSEELLSKYERGYHKKGADFDLNFCHQLIDFFKSSINKHEDWSKFNFKFSETNKYNDISEFYKEVELQGYKISFTDISEEYINKLVEEGKLYLFRIYNKDFSSYSKGTPNLHTLYWKALFDEDNLKNIIYKLNGGAEIFYRKKSIEAKITHPKNQPIDNKNPNNPKKQSIFEYDLIKDKRFTLDKFLFHVPITLNFKAKDTHKLNDLVNNKIKNTEDINIIGIDRGERHLLYACVINKNGEILEQYSLNTIGQTNYHYLLSKKEDDRLKQRQAWGTIQNIKELKEGYMSQVIHKLTKLMVQYNAVIVLEDLNSGFKNSRKKVEKSVYDKFETALINKLSYLVDKSIQNKFEQGGIMNAYQLANADIKSAKQNGVIFYIPAWCTSKIDPTTGFVNLFDLKKIDKEFVKKFNSIKFNNTENYFEFDFDYSNFSDKSSGDRTNWTLCSFGNRIRTFKNPKKNNNYDSQTVELTQEFKKLFDCYNLDCSNLRDEILNKSDSKFFNATPEKNNFYGFALLFKLLLQLRNSITGSEEDYILSPIKNKKGLFYDSRTANGVLPENADANGAYNIARKGLLILNRIKNSEENTKTDYTIKNADWLTYVQQQDR

SEQ ID NO: 82 - protein, 1224 aa, synthetic construct
MQLNPVGKTMDYFQAKQILENDEKLKENYQKIKEIADRFYRNLNEDVLSKTGLDKLKDYAEIYYHCNTDAERKRLDECASELRKEIVKNFKNRDEYNKLFNKKMIEIVLPQHLKNEDEKEVVASFKNFTTYFTGFFRNRKNMYSDGEESTAIAYRCINENLPKHLDNVKAFEKAISKLSKNAIDDLDATYSGLCGTNLYDVFTVDYFNFLLPQSGITEYNKIIGGYTTSDGTKVKGINEYINLYNQQVSKRDKIPNLKILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIREKSIVNYYKTSLMQLTDNLSDKYNEAAPLLNKSYANEKGLKNDDKSISLIKNFLDAIKEIEKFIKPLSETNITGEKNDLFYSQFTPLLDNISRIDILYDKVRNYVTQKPFSTDKIKLNFGNSQLLNGWDRNKEKDCGAVWLCRDEKYYLAIIDKSNNSILENIDFQDCDENDCYEKIIYKLLPGPNKMLPKVFFSEKCKKLLSPSDEILKIRKNGTFKKGDKFSLDDCHKLIDFYKESFKKYPNWLIYNFKFKNTNEYNDIREFYNDVASQGYNISKMKIPTSFIDKLVDEGKIYLFQLYNKDFSPHSKGTPNLHTLYFKMLFDERNLEDVVYKLNGEAEMFYRPASIKYDKPTHPKNTPIKNKNTLNDKKTSTFPYDLIKDKRYTKWQFSLHFPITMNFKAPDRAMINDDVRNLLKSCNNNFIIGIDRGERNLLYVSVIDSNGAIIYQHSLNIIGNKFKGKTYETNYREKLATREKERTEQRRNWKAIESIKELKEGYISQAVHVICQLVVKYDAIIVMEKLTDGFKRGRTKFEKQVYQKFEKMLIDKLNYYVDKKLDPDEGGGLLHAYQLTNKLESFDKLGMQSGFIFYVRPDFTSKIDPVTGFVNLLYPRYENIDKAKDMISRFDDIGYNAGEDFFEFDIDYDKFPKTASDYRKRWTICTNGERIEAFRNPAKNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKSDNVSTVGPVIHNDKWLKFVQENDMANN

SEQ ID NO: 83 - protein, 1224 aa, synthetic construct
MQLNPVGKTMDYFQAKQILENDEKLKENYQKIKEIADRFYRNLNEDVLSKTGLDKLKDYAEIYYHCNTDAERKRLDECASELRKEIVKNFKNRDEYNKLFNKKMIEIVLPQHLKNEDEKEVVASFKNFTTYFTGFFTNRKNMYSDGEESTAIAYRCINENLPKHLDNVKAFEKAISKLSKNAIDDLDATYSGLCGTNLYDVFTVDYFNFLLPQSGITEYNKIIGGYTTSDGTKVKGINEYINLYNQQVSKRDKIPNLKILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIREKSIVNYYKTSLMQLTDNLSDKYNEAAPLLNKSYANEKGLKNDDKSISLIKNFLDAIKEIEKFIKPLSETNITGEKNDLFYSQFTPLLDNISRIDILYDKVRNYVTQKPFSTDKIKLNFGNSQLLRGWDRNKEKDCGAVWLCRDEKYYLAIIDKSNNSILENIDFQDCDENDCYEKIIYKLLPGPNKMLPKVFFSEKCKKLLSPSDEILKIRKNGTFKKGDKFSLDDCHKLIDFYKESFKKYPNWLIYNFKFKNTNEYNDIREFYNDVASQGYNISKMKIPTSFIDKLVDEGKIYLFQLYNKDFSPHSKGTPNLHTLYFKMLFDERNLEDVVYKLNGEAEMFYRPASIKYDKPTHPKNTPIKNKNTLNDKKTSTFPYDLIKDKRYTKWQFSLHFPITMNFKAPDRAMINDDVRNLLKSCNNNFIIGIDRGERNLLYVSVIDSNGAIIYQHSLNIIGNKFKGKTYETNYREKLATREKERTEQRRNWKAIESIKELKEGYISQAVHVICQLVVKYDAIIVMEKLTDGFKRGRTKFEKQVYQKFEKMLIDKLNYYVDKKLDPDEGGGLLHAYQLTNKLESFDKLGMQSGFIFYVRPDFTSKIDPVTGFVNLLYPRYENIDKAKDMISRFDDIGYNAGEDFFEFDIDYDKFPKTASDYRKRWTICTNGERIEAFRNPAKNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKSDNVSTVGPVIHNDKWLKFVQENDMANN

SEQ ID NO: 84 - protein, 1224 aa, synthetic construct
MQLNPVGKTMDYFQAKQILENDEKLKENYQKIKEIADRFYRNLNEDVLSKTGLDKLKDYAEIYYHCNTDAERKRLDECASELRKEIVKNFKNRDEYNKLFNKKMIEIVLPQHLKNEDEKEVVASFKNFTTYFTGFFTNRKNMYSDGEESTAIAYRCINENLPKHLDNVKAFEKAISKLSKNAIDDLDATYSGLCGTNLYDVFTVDYFNFLLPQSGITEYNKIIGGYTTSDGTKVKGINEYINLYNQQVSKRDKIPNLKILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIREKSIVNYYKTSLMQLTDNLSDKYNEAAPLLNKSYANEKGLKNDDKSISLIKNFLDAIKEIEKFIKPLSETNITGEKNDLFYSQFTPLLDNISRIDILYDKVRNYVTQKPFSTDKIKLNFGNSQLLNGWDRNREKDCGAVWLCRDEKYYLAIIDKSNNSILENIDFQDCDENDCYEKIIYKLLPGPNKMLPKVFFSEKCKKLLSPSDEILKIRKNGTFKKGDKFSLDDCHKLIDFYKESFKKYPNWLIYNFKFKNTNEYNDIREFYNDVASQGYNISKMKIPTSFIDKLVDEGKIYLFQLYNKDFSPHSKGTPNLHTLYFKMLFDERNLEDVVYKLNGEAEMFYRPASIKYDKPTHPKNTPIKNKNTLNDKKTSTFPYDLIKDKRYTKWQFSLHFPITMNFKAPDRAMINDDVRNLLKSCNNNFIIGIDRGERNLLYVSVIDSNGAIIYQHSLNIIGNKFKGKTYETNYREKLATREKERTEQRRNWKAIESIKELKEGYISQAVHVICQLVVKYDAIIVMEKLTDGFKRGRTKFEKQVYQKFEKMLIDKLNYYVDKKLDPDEGGGLLHAYQLTNKLESFDKLGMQSGFIFYVRPDFTSKIDPVTGFVNLLYPRYENIDKAKDMISRFDDIGYNAGEDFFEFDIDYDKFPKTASDYRKRWTICTNGERIEAFRNPAKNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKSDNVSTVGPVIHNDKWLKFVQENDMANN

SEQ ID NO: 85 - protein, 1224 aa, synthetic construct
MQLNPVGKTMDYFQAKQILENDEKLKENYQKIKEIADRFYRNLNEDVLSKTGLDKLKDYAEIYYHCNTDAERKRLDECASELRKEIVKNFKNRDEYNKLFNKKMIEIVLPQHLKNEDEKEVVASFKNFTTYFTGFFRNRKNMYSDGEESTAIAYRCINENLPKHLDNVKAFEKAISKLSKNAIDDLDATYSGLCGTNLYDVFTVDYFNFLLPQSGITEYNKIIGGYTTSDGTKVKGINEYINLYNQQVSKRDKIPNLKILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIREKSIVNYYKTSLMQLTDNLSDKYNEAAPLLNKSYANEKGLKNDDKSISLIKNFLDAIKEIEKFIKPLSETNITGEKNDLFYSQFTPLLDNISRIDILYDKVRNYVTQKPFSTDKIKLNFGNSQLLRGWDRNKEKDCGAVWLCRDEKYYLAIIDKSNNSILENIDFQDCDENDCYEKIIYKLLPGPNKMLPKVFFSEKCKKLLSPSDEILKIRKNGTFKKGDKFSLDDCHKLIDFYKESFKKYPNWLIYNFKFKNTNEYNDIREFYNDVASQGYNISKMKIPTSFIDKLVDEGKIYLFQLYNKDFSPHSKGTPNLHTLYFKMLFDERNLEDVVYKLNGEAEMFYRPASIKYDKPTHPKNTPIKNKNTLNDKKTSTFPYDLIKDKRYTKWQFSLHFPITMNFKAPDRAMINDDVRNLLKSCNNNFIIGIDRGERNLLYVSVIDSNGAIIYQHSLNIIGNKFKGKTYETNYREKLATREKERTEQRRNWKAIESIKELKEGYISQAVHVICQLVVKYDAIIVMEKLTDGFKRGRTKFEKQVYQKFEKMLIDKLNYYVDKKLDPDEGGGLLHAYQLTNKLESFDKLGMQSGFIFYVRPDFTSKIDPVTGFVNLLYPRYENIDKAKDMISRFDDIGYNAGEDFFEFDIDYDKFPKTASDYRKRWTICTNGERIEAFRNPAKNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKSDNVSTVGPVIHNDKWLKFVQENDMANN

SEQ ID NO: 86 - protein, 1224 aa, synthetic construct
MQLNPVGKTMDYFQAKQILENDEKLKENYQKIKEIADRFYRNLNEDVLSKTGLDKLKDYAEIYYHCNTDAERKRLDECASELRKEIVKNFKNRDEYNKLFNKKMIEIVLPQHLKNEDEKEVVASFKNFTTYFTGFFRNRKNMYSDGEESTAIAYRCINENLPKHLDNVKAFEKAISKLSKNAIDDLDATYSGLCGTNLYDVFTVDYFNFLLPQSGITEYNKIIGGYTTSDGTKVKGINEYINLYNQQVSKRDKIPNLKILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIREKSIVNYYKTSLMQLTDNLSDKYNEAAPLLNKSYANEKGLKNDDKSISLIKNFLDAIKEIEKFIKPLSETNITGEKNDLFYSQFTPLLDNISRIDILYDKVRNYVTQKPFSTDKIKLNFGNSQLLNGWDRNREKDCGAVWLCRDEKYYLAIIDKSNNSILENIDFQDCDENDCYEKIIYKLLPGPNKMLPKVFFSEKCKKLLSPSDEILKIRKNGTFKKGDKFSLDDCHKLIDFYKESFKKYPNWLIYNFKFKNTNEYNDIREFYNDVASQGYNISKMKIPTSFIDKLVDEGKIYLFQLYNKDFSPHSKGTPNLHTLYFKMLFDERNLEDVVYKLNGEAEMFYRPASIKYDKPTHPKNTPIKNKNTLNDKKTSTFPYDLIKDKRYTKWQFSLHFPITMNFKAPDRAMINDDVRNLLKSCNNNFIIGIDRGERNLLYVSVIDSNGAIIYQHSLNIIGNKFKGKTYETNYREKLATREKERTEQRRNWKAIESIKELKEGYISQAVHVICQLVVKYDAIIVMEKLTDGFKRGRTKFEKQVYQKFEKMLIDKLNYYVDKKLDPDEGGGLLHAYQLTNKLESFDKLGMQSGFIFYVRPDFTSKIDPVTGFVNLLYPRYENIDKAKDMISRFDDIGYNAGEDFFEFDIDYDKFPKTASDYRKRWTICTNGERIEAFRNPAKNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKSDNVSTVGPVIHNDKWLKFVQENDMANN

SEQ ID NO: 87 - protein, 1224 aa, synthetic construct
MQLNPVGKTMDYFQAKQILENDEKLKENYQKIKEIADRFYRNLNEDVLSKTGLDKLKDYAEIYYHCNTDAERKRLDECASELRKEIVKNFKNRDEYNKLFNKKMIEIVLPQHLKNEDEKEVVASFKNFTTYFTGFFTNRKNMYSDGEESTAIAYRCINENLPKHLDNVKAFEKAISKLSKNAIDDLDATYSGLCGTNLYDVFTVDYFNFLLPQSGITEYNKIIGGYTTSDGTKVKGINEYINLYNQQVSKRDKIPNLKILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIREKSIVNYYKTSLMQLTDNLSDKYNEAAPLLNKSYANEKGLKNDDKSISLIKNFLDAIKEIEKFIKPLSETNITGEKNDLFYSQFTPLLDNISRIDILYDKVRNYVTQKPFSTDKIKLNFGNSQLLRGWDRNREKDCGAVWLCRDEKYYLAIIDKSNNSILENIDFQDCDENDCYEKIIYKLLPGPNKMLPKVFFSEKCKKLLSPSDEILKIRKNGTFKKGDKFSLDDCHKLIDFYKESFKKYPNWLIYNFKFKNTNEYNDIREFYNDVASQGYNISKMKIPTSFIDKLVDEGKIYLFQLYNKDFSPHSKGTPNLHTLYFKMLFDERNLEDVVYKLNGEAEMFYRPASIKYDKPTHPKNTPIKNKNTLNDKKTSTFPYDLIKDKRYTKWQFSLHFPITMNFKAPDRAMINDDVRNLLKSCNNNFIIGIDRGERNLLYVSVIDSNGAIIYQHSLNIIGNKFKGKTYETNYREKLATREKERTEQRRNWKAIESIKELKEGYISQAVHVICQLVVKYDAIIVMEKLTDGFKRGRTKFEKQVYQKFEKMLIDKLNYYVDKKLDPDEGGGLLHAYQLTNKLESFDKLGMQSGFIFYVRPDFTSKIDPVTGFVNLLYPRYENIDKAKDMISRFDDIGYNAGEDFFEFDIDYDKFPKTASDYRKRWTICTNGERIEAFRNPAKNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKSDNVSTVGPVIHNDKWLKFVQENDMANN

SEQ ID NO: 88 - protein, 1224 aa, synthetic construct
MQLNPVGKTMDYFQAKQILENDEKLKENYQKIKEIADRFYRNLNEDVLSKTGLDKLKDYAEIYYHCNTDAERKRLDECASELRKEIVKNFKNRDEYNKLFNKKMIEIVLPQHLKNEDEKEVVASFKNFTTYFTGFFRNRKNMYSDGEESTAIAYRCINENLPKHLDNVKAFEKAISKLSKNAIDDLDATYSGLCGTNLYDVFTVDYFNFLLPQSGITEYNKIIGGYTTSDGTKVKGINEYINLYNQQVSKRDKIPNLKILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIREKSIVNYYKTSLMQLTDNLSDKYNEAAPLLNKSYANEKGLKNDDKSISLIKNFLDAIKEIEKFIKPLSETNITGEKNDLFYSQFTPLLDNISRIDILYDKVRNYVTQKPFSTDKIKLNFGNSQLLRGWDRNREKDCGAVWLCRDEKYYLAIIDKSNNSILENIDFQDCDENDCYEKIIYKLLPGPNKMLPKVFFSEKCKKLLSPSDEILKIRKNGTFKKGDKFSLDDCHKLIDFYKESFKKYPNWLIYNFKFKNTNEYNDIREFYNDVASQGYNISKMKIPTSFIDKLVDEGKIYLFQLYNKDFSPHSKGTPNLHTLYFKMLFDERNLEDVVYKLNGEAEMFYRPASIKYDKPTHPKNTPIKNKNTLNDKKTSTFPYDLIKDKRYTKWQFSLHFPITMNFKAPDRAMINDDVRNLLKSCNNNFIIGIDRGERNLLYVSVIDSNGAIIYQHSLNIIGNKFKGKTYETNYREKLATREKERTEQRRNWKAIESIKELKEGYISQAVHVICQLVVKYDAIIVMEKLTDGFKRGRTKFEKQVYQKFEKMLIDKLNYYVDKKLDPDEGGGLLHAYQLTNKLESFDKLGMQSGFIFYVRPDFTSKIDPVTGFVNLLYPRYENIDKAKDMISRFDDIGYNAGEDFFEFDIDYDKFPKTASDYRKRWTICTNGERIEAFRNPAKNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKSDNVSTVGPVIHNDKWLKFVQENDMANN

SEQ ID NO: 89 - protein, 1224 aa, synthetic construct
MQLNPVGKTMDYFQAKQILENDEKLKENYQKIKEIADRFYRNLNEDVLSKTGLDKLKDYAEIYYHCNTDAERKRLDECASELRKEIVKNFKNRDEYNKLFNKKMIEIVLPQHLKNEDEKEVVASFKNFTTYFTGFFRNRKNMYSDGEESTAIAYRCINENLPKHLDNVKAFEKAISKLSKNAIDDLDATYSGLCGTNLYDVFTVDYFNFLLPQSGITEYNKIIGGYTTSDGTKVKGINEYIALYNQQVSKRDKIPNLKILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIREKSIVNYYKTSLMQLTDNLSDKYNEAAPLLNKSYANEKGLKNDDKSISLIKNFLDAIKEIEKFIKPLSETNITGEKNDLFYSQFTPLLDNISRIDILYDKVRNYVTQKPFSTDKIKLNFGNSQLLNGWDRNREKDCGAVWLCRDEKYYLAIIDKSNNSILENIDFQDCDENDCYEKIIYKLLPGPNKMLPKVFFSEKCKKLLSPSDEILKIRKNGTFKKGDKFSLDDCHKLIDFYKESFKKYPNWLIYNFKFKNTNEYNDIREFYNDVASQGYNISKMKIPTSFIDKLVDEGKIYLFQLYNKDFSPHSKGTPNLHTLYFKMLFDERNLEDVVYKLNGEAEMFYRPASIKYDKPTHPKNTPIKNKNTLNDKKTSTFPYDLIKDKRYTKWQFSLHFPITMNFKAPDRAMINDDVRNLLKSCNNNFIIGIDRGERNLLYVSVIDSNGAIIYQHSLNIIGNKFKGKTYETNYREKLATREKERTEQRRNWKAIESIKELKEGYISQAVHVICQLVVKYDAIIVMEKLTDGFKRGRTKFEKQVYQKFEKMLIDKLNYYVDKKLDPDEGGGLLHAYQLTNKLESFDKLGMQSGFIFYVRPDFTSKIDPVTGFVNLLYPRYENIDKAKDMISRFDDIGYNAGEDFFEFDIDYDKFPKTASDYRKRWTICTNGERIEAFRNPAKNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKSDNVSTVGPVIHNDKWLKFVQENDMANN

SEQ ID NO: 90 - protein, 1224 aa, synthetic construct
MQLNPVGKTMDYFQAKQILENDEKLKENYQKIKEIADRFYRNLNEDVLSKTGLDKLKDYAEIYYHCNTDAERKRLDECASELRKEIVKNFKNRDEYNKLFNKKMIEIVLPQHLKNEDEKEVVASFKNFTTYFTGFFTNRKNMYSDGEESTAIAYRCINENLPKHLDNVKAFEKAISKLSKNAIDDLDATYSGLCGTNLYDVFTVDYFNFLLPQSGITEYNKIIGGYTTSDGTKVKGINEYINLYNQQVSKRDKIPNLKILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIREKSIVNYYKTSLMQLTDNLSDKYNEAAPLLNKSYANEKGLKNDDKSISLIKNFLDAIKEIEKFIKPLSETNITGEKNDLFYSQFTPLLDNISRIDILYDKVRNYVTQKPFSTDKIKLNFGRSQLLNGWDRNKEKDCGAVWLCRDEKYYLAIIDKSNNSILENIDFQDCDENDCYEKIIYKLLPGPNKMLPKVFFSEKCKKLLSPSDEILKIRKNGTFKKGDKFSLDDCHKLIDFYKESFKKYPNWLIYNFKFKNTNEYNDIREFYNDVASQGYNISKMKIPTSFIDKLVDEGKIYLFQLYNKDFSPHSKGTPNLHTLYFKMLFDERNLEDVVYKLNGEAEMFYRPASIKYDKPTHPKNTPIKNKNTLNDKKTSTFPYDLIKDKRYTKWQFSLHFPITMNFKAPDRAMINDDVRNLLKSCNNNFIIGIDRGERNLLYVSVIDSNGAIIYQHSLNIIGNKFKGKTYETNYREKLATREKERTEQRRNWKAIESIKELKEGYISQAVHVICQLVVKYDAIIVMEKLTDGFKRGRTKFEKQVYQKFEKMLIDKLNYYVDKKLDPDEGGGLLHAYQLTNKLESFDKLGMQSGFIFYVRPDFTSKIDPVTGFVNLLYPRYENIDKAKDMISRFDDIGYNAGEDFFEFDIDYDKFPKTASDYRKRWTICTNGERIEAFRNPAKNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKSDNVSTVGPVIHNDKWLKFVQENDMANN

SEQ ID NO: 91 - protein, 1257 aa, synthetic construct
MNQNTSFSQFIGLYPVSKTLRFELKPIGKTLENVQNKGIIEQDEAKSLKYVEAKQIIDQYHKYFIGEALEGVNLDWNPLQKAFEDYLTNRNAPAKKNLEKTQESFRKEIAKKLSEHKHFKELTAATPKELFEKILPEHNGKQEAIEAFAKFSTYFKGFQRNRENIYSSDAISTGVPFRIVHDNFPKFLSSIQVFQNIKENCPCVLDDAEKELQELLKSQKLIDVFNINYFNNVISQDGIDFFNQVIGGYTTPNGTKVRGINEFANLYRQQNPEFAKKHIATKMIPLYKQILSDRESMSFVLESFNKDTDVQDGVKEFFEHHLLRCNMDGNEINVLEKIGELILGLKNYDSLNKFFVSRDSLSSISQKLFDNWNTLNNYLFNYWENKIGSVEKASNKKRIDAKLKEDYFSIEELNEVLKFNESDANICDFWTDYTSQANSVLSGEIKKNYEEFLAADYSNKALRENEADVQKLKDFLDAIVNILHLVKPLMMDSDQGEPSFYNELNPLYDQLSSIIPLYNKTRNYITQKISDSTKKFKLNFENPTLADGWDQNKEEANTSIILKKNEKFYLGIMNAKDKPKIGLYKVKPEEPHYDKMVYKLLPGPNKMLPKVFFSAKGKEIYKPSKEIQDGYAAGKHKKGDSFDKQFCHHLIDFFKEGISNHPDWKNFNFKFSETSSYEDISAFYNEVSDQGYKLSFFPIPDSQIDAWVNEGKLFLFQIYNKDFAPGAKGKPNLHTLYWKATFSLENLKDIVFKLNGEAELFYRRSSIKKPYSHKIGEKMVNRITKDGRPIPDAIFGELFRYFNNSSKTSLSDEAKKYLDFVTVKDVKHEIIKDKRYTEDKFEFHVPITINFKADDGSRYINDQVKDFLKNNSDVNIIGIDRGERNLIYMTLINQKGEILAQKSFNLVGNTNYHEKLSIREQERDAARRSWQSIGKIKELKEGYLSLVIHEIAKTMIENNAIIVLEDLNFGFKRGRFCVEKQVYQKFEKMLIDKLNYLVFKDCADSEYGGILKGYQLTQKFTSFKDIGKQNGFLFYVPAAYTSKIDPTTGFVNLFNFRDLTNAEKKKDFLTTLDDITFNSETNSFAFTFDYSKFKVFQTDYQKIWTVFTNGERIVYDRESKKHNIIEPTTIIQEALEKQGIQCVDQLNVLAEIEKIEPTRENARLFDSICYAFEKTLQMRNSNSETGDDYILSPVKNKNGVFFNSNEAGDKLPKDADANGAFHIALKGLFLLQHISETDEKLKIPHEKWFEFVQSRNK

SEQ ID NO: 92 - protein, 1257 aa, synthetic construct
MNQNTSFSQFIGLYPVSKTLRFELKPIGKTLENVQNKGIIEQDEAKSLKYVEAKQIIDQYHKYFIGEALEGVNLDWNPLQKAFEDYLTNRNAPAKKNLEKTQESFRKEIAKKLSEHKHFKELTAATPKELFEKILPEHNGKQEAIEAFAKFSTYFKGFQENRENIYSSDAISTGVPFRIVHDNFPKFLSSIQVFQNIKENCPCVLDDAEKELQELLKSQKLIDVFNINYFNNVISQDGIDFFNQVIGGYTTPNGTKVRGINEFANLYRQQNPEFAKKHIATKMIPLYKQILSDRESMSFVLESFNKDTDVQDGVKEFFEHHLLRCNMDGNEINVLEKIGELILGLKNYDSLNKFFVSRDSLSSISQKLFDNWNTLNNYLFNYWENKIGSVEKASNKKRIDAKLKEDYFSIEELNEVLKFNESDANICDFWTDYTSQANSVLSGEIKKNYEEFLAADYSNKALRENEADVQKLKDFLDAIVNILHLVKPLMMDSDQGEPSFYNELNPLYDQLSSIIPLYNKTRNYITQKISDSTKKFKLNFENPTLARGWDQNKEEANTSIILKKNEKFYLGIMNAKDKPKIGLYKVKPEEPHYDKMVYKLLPGPNKMLPKVFFSAKGKEIYKPSKEIQDGYAAGKHKKGDSFDKQFCHHLIDFFKEGISNHPDWKNFNFKFSETSSYEDISAFYNEVSDQGYKLSFFPIPDSQIDAWVNEGKLFLFQIYNKDFAPGAKGKPNLHTLYWKATFSLENLKDIVFKLNGEAELFYRRSSIKKPYSHKIGEKMVNRITKDGRPIPDAIFGELFRYFNNSSKTSLSDEAKKYLDFVTVKDVKHEIIKDKRYTEDKFEFHVPITINFKADDGSRYINDQVKDFLKNNSDVNIIGIDRGERNLIYMTLINQKGEILAQKSFNLVGNTNYHEKLSIREQERDAARRSWQSIGKIKELKEGYLSLVIHEIAKTMIENNAIIVLEDLNFGFKRGRFCVEKQVYQKFEKMLIDKLNYLVFKDCADSEYGGILKGYQLTQKFTSFKDIGKQNGFLFYVPAAYTSKIDPTTGFVNLFNFRDLTNAEKKKDFLTTLDDITFNSETNSFAFTFDYSKFKVFQTDYQKIWTVFTNGERIVYDRESKKHNIIEPTTIIQEALEKQGIQCVDQLNVLAEIEKIEPTRENARLFDSICYAFEKTLQMRNSNSETGDDYILSPVKNKNGVFFNSNEAGDKLPKDADANGAFHIALKGLFLLQHISETDEKLKIPHEKWFEFVQSRNK

SEQ ID NO: 93 - protein, 1257 aa, synthetic construct
MNQNTSFSQFIGLYPVSKTLRFELKPIGKTLENVQNKGIIEQDEAKSLKYVEAKQIIDQYHKYFIGEALEGVNLDWNPLQKAFEDYLTNRNAPAKKNLEKTQESFRKEIAKKLSEHKHFKELTAATPKELFEKILPEHNGKQEAIEAFAKFSTYFKGFQENRENIYSSDAISTGVPFRIVHDNFPKFLSSIQVFQNIKENCPCVLDDAEKELQELLKSQKLIDVFNINYFNNVISQDGIDFFNQVIGGYTTPNGTKVRGINEFANLYRQQNPEFAKKHIATKMIPLYKQILSDRESMSFVLESFNKDTDVQDGVKEFFEHHLLRCNMDGNEINVLEKIGELILGLKNYDSLNKFFVSRDSLSSISQKLFDNWNTLNNYLFNYWENKIGSVEKASNKKRIDAKLKEDYFSIEELNEVLKFNESDANICDFWTDYTSQANSVLSGEIKKNYEEFLAADYSNKALRENEADVQKLKDFLDAIVNILHLVKPLMMDSDQGEPSFYNELNPLYDQLSSIIPLYNKTRNYITQKISDSTKKFKLNFENPTLADGWDQNREEANTSIILKKNEKFYLGIMNAKDKPKIGLYKVKPEEPHYDKMVYKLLPGPNKMLPKVFFSAKGKEIYKPSKEIQDGYAAGKHKKGDSFDKQFCHHLIDFFKEGISNHPDWKNFNFKFSETSSYEDISAFYNEVSDQGYKLSFFPIPDSQIDAWVNEGKLFLFQIYNKDFAPGAKGKPNLHTLYWKATFSLENLKDIVFKLNGEAELFYRRSSIKKPYSHKIGEKMVNRITKDGRPIPDAIFGELFRYFNNSSKTSLSDEAKKYLDFVTVKDVKHEIIKDKRYTEDKFEFHVPITINFKADDGSRYINDQVKDFLKNNSDVNIIGIDRGERNLIYMTLINQKGEILAQKSFNLVGNTNYHEKLSIREQERDAARRSWQSIGKIKELKEGYLSLVIHEIAKTMIENNAIIVLEDLNFGFKRGRFCVEKQVYQKFEKMLIDKLNYLVFKDCADSEYGGILKGYQLTQKFTSFKDIGKQNGFLFYVPAAYTSKIDPTTGFVNLFNFRDLTNAEKKKDFLTTLDDITFNSETNSFAFTFDYSKFKVFQTDYQKIWTVFTNGERIVYDRESKKHNIIEPTTIIQEALEKQGIQCVDQLNVLAEIEKIEPTRENARLFDSICYAFEKTLQMRNSNSETGDDYILSPVKNKNGVFFNSNEAGDKLPKDADANGAFHIALKGLFLLQHISETDEKLKIPHEKWFEFVQSRNK

SEQ ID NO: 94 - protein, 1257 aa, synthetic construct
MNQNTSFSQFIGLYPVSKTLRFELKPIGKTLENVQNKGIIEQDEAKSLKYVEAKQIIDQYHKYFIGEALEGVNLDWNPLQKAFEDYLTNRNAPAKKNLEKTQESFRKEIAKKLSEHKHFKELTAATPKELFEKILPEHNGKQEAIEAFAKFSTYFKGFQRNRENIYSSDAISTGVPFRIVHDNFPKFLSSIQVFQNIKENCPCVLDDAEKELQELLKSQKLIDVFNINYFNNVISQDGIDFFNQVIGGYTTPNGTKVRGINEFANLYRQQNPEFAKKHIATKMIPLYKQILSDRESMSFVLESFNKDTDVQDGVKEFFEHHLLRCNMDGNEINVLEKIGELILGLKNYDSLNKFFVSRDSLSSISQKLFDNWNTLNNYLFNYWENKIGSVEKASNKKRIDAKLKEDYFSIEELNEVLKFNESDANICDFWTDYTSQANSVLSGEIKKNYEEFLAADYSNKALRENEADVQKLKDFLDAIVNILHLVKPLMMDSDQGEPSFYNELNPLYDQLSSIIPLYNKTRNYITQKISDSTKKFKLNFENPTLARGWDQNKEEANTSIILKKNEKFYLGIMNAKDKPKIGLYKVKPEEPHYDKMVYKLLPGPNKMLPKVFFSAKGKEIYKPSKEIQDGYAAGKHKKGDSFDKQFCHHLIDFFKEGISNHPDWKNFNFKFSETSSYEDISAFYNEVSDQGYKLSFFPIPDSQIDAWVNEGKLFLFQIYNKDFAPGAKGKPNLHTLYWKATFSLENLKDIVFKLNGEAELFYRRSSIKKPYSHKIGEKMVNRITKDGRPIPDAIFGELFRYFNNSSKTSLSDEAKKYLDFVTVKDVKHEIIKDKRYTEDKFEFHVPITINFKADDGSRYINDQVKDFLKNNSDVNIIGIDRGERNLIYMTLINQKGEILAQKSFNLVGNTNYHEKLSIREQERDAARRSWQSIGKIKELKEGYLSLVIHEIAKTMIENNAIIVLEDLNFGFKRGRFCVEKQVYQKFEKMLIDKLNYLVFKDCADSEYGGILKGYQLTQKFTSFKDIGKQNGFLFYVPAAYTSKIDPTTGFVNLFNFRDLTNAEKKKDFLTTLDDITFNSETNSFAFTFDYSKFKVFQTDYQKIWTVFTNGERIVYDRESKKHNIIEPTTIIQEALEKQGIQCVDQLNVLAEIEKIEPTRENARLFDSICYAFEKTLQMRNSNSETGDDYILSPVKNKNGVFFNSNEAGDKLPKDADANGAFHIALKGLFLLQHISETDEKLKIPHEKWFEFVQSRNK

SEQ ID NO: 95 - protein, 1257 aa, synthetic construct
MNQNTSFSQFIGLYPVSKTLRFELKPIGKTLENVQNKGIIEQDEAKSLKYVEAKQIIDQYHKYFIGEALEGVNLDWNPLQKAFEDYLTNRNAPAKKNLEKTQESFRKEIAKKLSEHKHFKELTAATPKELFEKILPEHNGKQEAIEAFAKFSTYFKGFQRNRENIYSSDAISTGVPFRIVHDNFPKFLSSIQVFQNIKENCPCVLDDAEKELQELLKSQKLIDVFNINYFNNVISQDGIDFFNQVIGGYTTPNGTKVRGINEFANLYRQQNPEFAKKHIATKMIPLYKQILSDRESMSFVLESFNKDTDVQDGVKEFFEHHLLRCNMDGNEINVLEKIGELILGLKNYDSLNKFFVSRDSLSSISQKLFDNWNTLNNYLFNYWENKIGSVEKASNKKRIDAKLKEDYFSIEELNEVLKFNESDANICDFWTDYTSQANSVLSGEIKKNYEEFLAADYSNKALRENEADVQKLKDFLDAIVNILHLVKPLMMDSDQGEPSFYNELNPLYDQLSSIIPLYNKTRNYITQKISDSTKKFKLNFENPTLADGWDQNREEANTSIILKKNEKFYLGIMNAKDKPKIGLYKVKPEEPHYDKMVYKLLPGPNKMLPKVFFSAKGKEIYKPSKEIQDGYAAGKHKKGDSFDKQFCHHLIDFFKEGISNHPDWKNFNFKFSETSSYEDISAFYNEVSDQGYKLSFFPIPDSQIDAWVNEGKLFLFQIYNKDFAPGAKGKPNLHTLYWKATFSLENLKDIVFKLNGEAELFYRRSSIKKPYSHKIGEKMVNRITKDGRPIPDAIFGELFRYFNNSSKTSLSDEAKKYLDFVTVKDVKHEIIKDKRYTEDKFEFHVPITINFKADDGSRYINDQVKDFLKNNSDVNIIGIDRGERNLIYMTLINQKGEILAQKSFNLVGNTNYHEKLSIREQERDAARRSWQSIGKIKELKEGYLSLVIHEIAKTMIENNAIIVLEDLNFGFKRGRFCVEKQVYQKFEKMLIDKLNYLVFKDCADSEYGGILKGYQLTQKFTSFKDIGKQNGFLFYVPAAYTSKIDPTTGFVNLFNFRDLTNAEKKKDFLTTLDDITFNSETNSFAFTFDYSKFKVFQTDYQKIWTVFTNGERIVYDRESKKHNIIEPTTIIQEALEKQGIQCVDQLNVLAEIEKIEPTRENARLFDSICYAFEKTLQMRNSNSETGDDYILSPVKNKNGVFFNSNEAGDKLPKDADANGAFHIALKGLFLLQHISETDEKLKIPHEKWFEFVQSRNK

SEQ ID NO: 96 - protein, 1257 aa, synthetic construct
MNQNTSFSQFIGLYPVSKTLRFELKPIGKTLENVQNKGIIEQDEAKSLKYVEAKQIIDQYHKYFIGEALEGVNLDWNPLQKAFEDYLTNRNAPAKKNLEKTQESFRKEIAKKLSEHKHFKELTAATPKELFEKILPEHNGKQEAIEAFAKFSTYFKGFQENRENIYSSDAISTGVPFRIVHDNFPKFLSSIQVFQNIKENCPCVLDDAEKELQELLKSQKLIDVFNINYFNNVISQDGIDFFNQVIGGYTTPNGTKVRGINEFANLYRQQNPEFAKKHIATKMIPLYKQILSDRESMSFVLESFNKDTDVQDGVKEFFEHHLLRCNMDGNEINVLEKIGELILGLKNYDSLNKFFVSRDSLSSISQKLFDNWNTLNNYLFNYWENKIGSVEKASNKKRIDAKLKEDYFSIEELNEVLKFNESDANICDFWTDYTSQANSVLSGEIKKNYEEFLAADYSNKALRENEADVQKLKDFLDAIVNILHLVKPLMMDSDQGEPSFYNELNPLYDQLSSIIPLYNKTRNYITQKISDSTKKFKLNFENPTLARGWDQNREEANTSIILKKNEKFYLGIMNAKDKPKIGLYKVKPEEPHYDKMVYKLLPGPNKMLPKVFFSAKGKEIYKPSKEIQDGYAAGKHKKGDSFDKQFCHHLIDFFKEGISNHPDWKNFNFKFSETSSYEDISAFYNEVSDQGYKLSFFPIPDSQIDAWVNEGKLFLFQIYNKDFAPGAKGKPNLHTLYWKATFSLENLKDIVFKLNGEAELFYRRSSIKKPYSHKIGEKMVNRITKDGRPIPDAIFGELFRYFNNSSKTSLSDEAKKYLDFVTVKDVKHEIIKDKRYTEDKFEFHVPITINFKADDGSRYINDQVKDFLKNNSDVNIIGIDRGERNLIYMTLINQKGEILAQKSFNLVGNTNYHEKLSIREQERDAARRSWQSIGKIKELKEGYLSLVIHEIAKTMIENNAIIVLEDLNFGFKRGRFCVEKQVYQKFEKMLIDKLNYLVFKDCADSEYGGILKGYQLTQKFTSFKDIGKQNGFLFYVPAAYTSKIDPTTGFVNLFNFRDLTNAEKKKDFLTTLDDITFNSETNSFAFTFDYSKFKVFQTDYQKIWTVFTNGERIVYDRESKKHNIIEPTTIIQEALEKQGIQCVDQLNVLAEIEKIEPTRENARLFDSICYAFEKTLQMRNSNSETGDDYILSPVKNKNGVFFNSNEAGDKLPKDADANGAFHIALKGLFLLQHISETDEKLKIPHEKWFEFVQSRNK

SEQ ID NO: 97 - protein, 1257 aa, synthetic construct
MNQNTSFSQFIGLYPVSKTLRFELKPIGKTLENVQNKGIIEQDEAKSLKYVEAKQIIDQYHKYFIGEALEGVNLDWNPLQKAFEDYLTNRNAPAKKNLEKTQESFRKEIAKKLSEHKHFKELTAATPKELFEKILPEHNGKQEAIEAFAKFSTYFKGFQRNRENIYSSDAISTGVPFRIVHDNFPKFLSSIQVFQNIKENCPCVLDDAEKELQELLKSQKLIDVFNINYFNNVISQDGIDFFNQVIGGYTTPNGTKVRGINEFANLYRQQNPEFAKKHIATKMIPLYKQILSDRESMSFVLESFNKDTDVQDGVKEFFEHHLLRCNMDGNEINVLEKIGELILGLKNYDSLNKFFVSRDSLSSISQKLFDNWNTLNNYLFNYWENKIGSVEKASNKKRIDAKLKEDYFSIEELNEVLKFNESDANICDFWTDYTSQANSVLSGEIKKNYEEFLAADYSNKALRENEADVQKLKDFLDAIVNILHLVKPLMMDSDQGEPSFYNELNPLYDQLSSIIPLYNKTRNYITQKISDSTKKFKLNFENPTLARGWDQNREEANTSIILKKNEKFYLGIMNAKDKPKIGLYKVKPEEPHYDKMVYKLLPGPNKMLPKVFFSAKGKEIYKPSKEIQDGYAAGKHKKGDSFDKQFCHHLIDFFKEGISNHPDWKNFNFKFSETSSYEDISAFYNEVSDQGYKLSFFPIPDSQIDAWVNEGKLFLFQIYNKDFAPGAKGKPNLHTLYWKATFSLENLKDIVFKLNGEAELFYRRSSIKKPYSHKIGEKMVNRITKDGRPIPDAIFGELFRYFNNSSKTSLSDEAKKYLDFVTVKDVKHEIIKDKRYTEDKFEFHVPITINFKADDGSRYINDQVKDFLKNNSDVNIIGIDRGERNLIYMTLINQKGEILAQKSFNLVGNTNYHEKLSIREQERDAARRSWQSIGKIKELKEGYLSLVIHEIAKTMIENNAIIVLEDLNFGFKRGRFCVEKQVYQKFEKMLIDKLNYLVFKDCADSEYGGILKGYQLTQKFTSFKDIGKQNGFLFYVPAAYTSKIDPTTGFVNLFNFRDLTNAEKKKDFLTTLDDITFNSETNSFAFTFDYSKFKVFQTDYQKIWTVFTNGERIVYDRESKKHNIIEPTTIIQEALEKQGIQCVDQLNVLAEIEKIEPTRENARLFDSICYAFEKTLQMRNSNSETGDDYILSPVKNKNGVFFNSNEAGDKLPKDADANGAFHIALKGLFLLQHISETDEKLKIPHEKWFEFVQSRNK

SEQ ID NO: 98 - protein, 1257 aa, synthetic construct
MNQNTSFSQFIGLYPVSKTLRFELKPIGKTLENVQNKGIIEQDEAKSLKYVEAKQIIDQYHKYFIGEALEGVNLDWNPLQKAFEDYLTNRNAPAKKNLEKTQESFRKEIAKKLSEHKHFKELTAATPKELFEKILPEHNGKQEAIEAFAKFSTYFKGFQRNRENIYSSDAISTGVPFRIVHDNFPKFLSSIQVFQNIKENCPCVLDDAEKELQELLKSQKLIDVFNINYFNNVISQDGIDFFNQVIGGYTTPNGTKVRGINEFAALYRQQNPEFAKKHIATKMIPLYKQILSDRESMSFVLESFNKDTDVQDGVKEFFEHHLLRCNMDGNEINVLEKIGELILGLKNYDSLNKFFVSRDSLSSISQKLFDNWNTLNNYLFNYWENKIGSVEKASNKKRIDAKLKEDYFSIEELNEVLKFNESDANICDFWTDYTSQANSVLSGEIKKNYEEFLAADYSNKALRENEADVQKLKDFLDAIVNILHLVKPLMMDSDQGEPSFYNELNPLYDQLSSIIPLYNKTRNYITQKISDSTKKFKLNFENPTLADGWDQNREEANTSIILKKNEKFYLGIMNAKDKPKIGLYKVKPEEPHYDKMVYKLLPGPNKMLPKVFFSAKGKEIYKPSKEIQDGYAAGKHKKGDSFDKQFCHHLIDFFKEGISNHPDWKNFNFKFSETSSYEDISAFYNEVSDQGYKLSFFPIPDSQIDAWVNEGKLFLFQIYNKDFAPGAKGKPNLHTLYWKATFSLENLKDIVFKLNGEAELFYRRSSIKKPYSHKIGEKMVNRITKDGRPIPDAIFGELFRYFNNSSKTSLSDEAKKYLDFVTVKDVKHEIIKDKRYTEDKFEFHVPITINFKADDGSRYINDQVKDFLKNNSDVNIIGIDRGERNLIYMTLINQKGEILAQKSFNLVGNTNYHEKLSIREQERDAARRSWQSIGKIKELKEGYLSLVIHEIAKTMIENNAIIVLEDLNFGFKRGRFCVEKQVYQKFEKMLIDKLNYLVFKDCADSEYGGILKGYQLTQKFTSFKDIGKQNGFLFYVPAAYTSKIDPTTGFVNLFNFRDLTNAEKKKDFLTTLDDITFNSETNSFAFTFDYSKFKVFQTDYQKIWTVFTNGERIVYDRESKKHNIIEPTTIIQEALEKQGIQCVDQLNVLAEIEKIEPTRENARLFDSICYAFEKTLQMRNSNSETGDDYILSPVKNKNGVFFNSNEAGDKLPKDADANGAFHIALKGLFLLQHISETDEKLKIPHEKWFEFVQSRNK

SEQ ID NO: 99 - protein, 1257 aa, synthetic construct
MNQNTSFSQFIGLYPVSKTLRFELKPIGKTLENVQNKGIIEQDEAKSLKYVEAKQIIDQYHKYFIGEALEGVNLDWNPLQKAFEDYLTNRNAPAKKNLEKTQESFRKEIAKKLSEHKHFKELTAATPKELFEKILPEHNGKQEAIEAFAKFSTYFKGFQENRENIYSSDAISTGVPFRIVHDNFPKFLSSIQVFQNIKENCPCVLDDAEKELQELLKSQKLIDVFNINYFNNVISQDGIDFFNQVIGGYTTPNGTKVRGINEFANLYRQQNPEFAKKHIATKMIPLYKQILSDRESMSFVLESFNKDTDVQDGVKEFFEHHLLRCNMDGNEINVLEKIGELILGLKNYDSLNKFFVSRDSLSSISQKLFDNWNTLNNYLFNYWENKIGSVEKASNKKRIDAKLKEDYFSIEELNEVLKFNESDANICDFWTDYTSQANSVLSGEIKKNYEEFLAADYSNKALRENEADVQKLKDFLDAIVNILHLVKPLMMDSDQGEPSFYNELNPLYDQLSSIIPLYNKTRNYITQKISDSTKKFKLNFERPTLADGWDQNKEEANTSIILKKNEKFYLGIMNAKDKPKIGLYKVKPEEPHYDKMVYKLLPGPNKMLPKVFFSAKGKEIYKPSKEIQDGYAAGKHKKGDSFDKQFCHHLIDFFKEGISNHPDWKNFNFKFSETSSYEDISAFYNEVSDQGYKLSFFPIPDSQIDAWVNEGKLFLFQIYNKDFAPGAKGKPNLHTLYWKATFSLENLKDIVFKLNGEAELFYRRSSIKKPYSHKIGEKMVNRITKDGRPIPDAIFGELFRYFNNSSKTSLSDEAKKYLDFVTVKDVKHEIIKDKRYTEDKFLFHVPITINFKADDGSRYINDQVKDFLKNNSDVNIIGIDRGERNLIYMTLINQKGEILAQKSFNLVGNTNYHEKLSIREQERDAARRSWQSIGKIKELKEGYLSLVIHEIAKTMIENNAIIVLEDLNFGFKRGRFCVEKQVYQKFEKMLIDKLNYLVFKDCADSEYGGILKGYQLTQKFTSFKDIGKQNGFLFYVPAAYTSKIDPTTGFVNLFNFRDLTNAEKKKDFLTTLDDITFNSETNSFAFTFDYSKFKVFQTDYQKIWTVFTNGERIVYDRESKKHNIIEPTTIIQEALEKQGIQCVDQLNVLAEIEKIEPTRENARLFDSICYAFEKTLQMRNSNSETGDDYILSPVKNKNGVFFNSNEAGDKLPKDADANGAFHIALKGLFLLQHISETDEKLKIPHEKWFEFVQSRNK

SEQ ID NO: 100 - protein, 1205 aa, synthetic construct
MKLEDFTNLYSLSKTLRFELRPIGKTRENIENGGLLRQDEDRAEKYVHIKKLIDEYHKAYIDKQLSGLVLQYADIGKANSLEEYYHSTRKSKDSDKDKIVKIQDNLRKQIVKRLKDSDEFKRIDKKELIQSDLAEFIKPAEDRALIAEFKNFTTYFTGFNRNRQNMYSDKAISTAIAYRLIHENLPKFIDNIETFDRIAGITELYDQTSSDAEIFRLAHFSETLSQKQIDAYNSVMGRYNMLINEYNQTHKQSRLPKFKMLYKQILSDREHPSWLPEQFESDTAVLTAIRECYDDLRIPMANLKTLLEGLGNYDPSGIFLRNDQHLSQISKRLTGDRSSIERSVTEDLLTSRKLNKRKSRTTDEEESRKLFKQKGSLSIGYIADTAKIDVERYFAKLGAINTVTEQSENLFAKAENARTTADELLANDYPAGKRLVQSNDDIALVKNLLDALMELQWFVKPLLGTGDEAGKDERFYGEFTQIWEQLDRITPLYNMVRNYVTRKPYSTDKFKLNFESAALLGGWDKNKEPDCLSVILRKDEQYYLGIINKNHKKIFARDILPCEGECYEKMVYKQISISAGIGGFFRKCYRTAEQYGWCCSDSCLNDERKIIIRDDEARENLTEIINCQKDFLNKYEKDGFKYRDFGFRFAASSDYEKLSDFYRDIDEQRYKLSFTPVSATYIDSLVEEGKLYLFQIYNKDFSAYSKGTPNMHTLYWRMLFDERNLSDVVYQLNGGAELFFRRKSLQNGRPTHPANIPIKNKNSRNDKKESLFDYDLIKDRRYTVDKFQFHVPITLNFKSDGAGRINERVREYLRSADDVHVIGIDRGERNLLYLVVTDMDGNICEQFSLNEICNTDYHSLLDEREHKRMQERQSWQAIEGIKELKEGYLSQVVHRIATLMVKYRAIVVLEDLNFGFMRSRQKVEKSVYQKFEHMLIDKLNYLVDKKANPTTPGGLLKAYQLTDKFESFQKLGKQSGFLFYVPAWNTSKIDPATGFVNMLDLGYESIDKAKALLCKFDSIRYNACKDWFEFALDYDKFGSKATGTRTKWTVCTYGQRIDTYRNKDSQWVSRDVDLTNELKSLFSEHGIDIYSNLKDAIVAQNDKEFFANMLRILKLTMQMRNSKTGTDIDYIVSPVADANGRFFDSRQADATMPKDADANGAYNIARKGIMLVQQIKQSDDLRTMKFDISNKSWLRFVQHTNQADE

SEQ ID NO: 101 - protein, 1205 aa, synthetic construct
MKLEDFTNLYSLSKTLRFELRPIGKTRENIENGGLLRQDEDRAEKYVHIKKLIDEYHKAYIDKQLSGLVLQYADIGKANSLEEYYHSTRKSKDSDKDKIVKIQDNLRKQIVKRLKDSDEFKRIDKKELIQSDLAEFIKPAEDRALIAEFKNFTTYFTGFNENRQNMYSDKAISTAIAYRLIHENLPKFIDNIETFDRIAGITELYDQTSSDAEIFRLAHFSETLSQKQIDAYNSVMGRYNMLINEYNQTHKQSRLPKFKMLYKQILSDREHPSWLPEQFESDTAVLTAIRECYDDLRIPMANLKTLLEGLGNYDPSGIFLRNDQHLSQISKRLTGDRSSIERSVTEDLLTSRKLNKRKSRTTDEEESRKLFKQKGSLSIGYIADTAKIDVERYFAKLGAINTVTEQSENLFAKAENARTTADELLANDYPAGKRLVQSNDDIALVKNLLDALMELQWFVKPLLGTGDEAGKDERFYGEFTQIWEQLDRITPLYNMVRNYVTRKPYSTDKFKLNFESAALLRGWDKNKEPDCLSVILRKDEQYYLGIINKNHKKIFARDILPCEGECYEKMVYKQISISAGIGGFFRKCYRTAEQYGWCCSDSCLNDERKIIIRDDEARENLTEIINCQKDFLNKYEKDGFKYRDFGFRFAASSDYEKLSDFYRDIDEQRYKLSFTPVSATYIDSLVEEGKLYLFQIYNKDFSAYSKGTPNMHTLYWRMLFDERNLSDVVYQLNGGAELFFRRKSLQNGRPTHPANIPIKNKNSRNDKKESLFDYDLIKDRRYTVDKFQFHVPITLNFKSDGAGRINERVREYLRSADDVHVIGIDRGERNLLYLVVTDMDGNICEQFSLNEICNTDYHSLLDEREHKRMQERQSWQAIEGIKELKEGYLSQVVHRIATLMVKYRAIVVLEDLNFGFMRSRQKVEKSVYQKFEHMLIDKLNYLVDKKANPTTPGGLLKAYQLTDKFESFQKLGKQSGFLFYVPAWNTSKIDPATGFVNMLDLGYESIDKAKALLCKFDSIRYNACKDWFEFALDYDKFGSKATGTRTKWTVCTYGQRIDTYRNKDSQWVSRDVDLTNELKSLFSEHGIDIYSNLKDAIVAQNDKEFFANMLRILKLTMQMRNSKTGTDIDYIVSPVADANGRFFDSRQADATMPKDADANGAYNIARKGIMLVQQIKQSDDLRTMKFDISNKSWLRFVQHTNQADE

SEQ ID NO: 102 - protein, 1205 aa, synthetic construct
MKLEDFTNLYSLSKTLRFELRPIGKTRENIENGGLLRQDEDRAEKYVHIKKLIDEYHKAYIDKQLSGLVLQYADIGKANSLEEYYHSTRKSKDSDKDKIVKIQDNLRKQIVKRLKDSDEFKRIDKKELIQSDLAEFIKPAEDRALIAEFKNFTTYFTGFNRNRQNMYSDKAISTAIAYRLIHENLPKFIDNIETFDRIAGITELYDQTSSDAEIFRLAHFSETLSQKQIDAYNSVMGRYNMLINEYNQTHKQSRLPKFKMLYKQILSDREHPSWLPEQFESDTAVLTAIRECYDDLRIPMANLKTLLEGLGNYDPSGIFLRNDQHLSQISKRLTGDRSSIERSVTEDLLTSRKLNKRKSRTTDEEESRKLFKQKGSLSIGYIADTAKIDVERYFAKLGAINTVTEQSENLFAKAENARTTADELLANDYPAGKRLVQSNDDIALVKNLLDALMELQWFVKPLLGTGDEAGKDERFYGEFTQIWEQLDRITPLYNMVRNYVTRKPYSTDKFKLNFESAALLRGWDKNKEPDCLSVILRKDEQYYLGIINKNHKKIFARDILPCEGECYEKMVYKQISISAGIGGFFRKCYRTAEQYGWCCSDSCLNDERKIIIRDDEARENLTEIINCQKDFLNKYEKDGFKYRDFGFRFAASSDYEKLSDFYRDIDEQRYKLSFTPVSATYIDSLVEEGKLYLFQIYNKDFSAYSKGTPNMHTLYWRMLFDERNLSDVVYQLNGGAELFFRRKSLQNGRPTHPANIPIKNKNSRNDKKESLFDYDLIKDRRYTVDKFQFHVPITLNFKSDGAGRINERVREYLRSADDVHVIGIDRGERNLLYLVVTDMDGNICEQFSLNEICNTDYHSLLDEREHKRMQERQSWQAIEGIKELKEGYLSQVVHRIATLMVKYRAIVVLEDLNFGFMRSRQKVEKSVYQKFEHMLIDKLNYLVDKKANPTTPGGLLKAYQLTDKFESFQKLGKQSGFLFYVPAWNTSKIDPATGFVNMLDLGYESIDKAKALLCKFDSIRYNACKDWFEFALDYDKFGSKATGTRTKWTVCTYGQRIDTYRNKDSQWVSRDVDLTNELKSLFSEHGIDIYSNLKDAIVAQNDKEFFANMLRILKLTMQMRNSKTGTDIDYIVSPVADANGRFFDSRQADATMPKDADANGAYNIARKGIMLVQQIKQSDDLRTMKFDISNKSWLRFVQHTNQADE

SEQ ID NO: 103 - protein, 1205 aa, synthetic construct; identical to SEQ ID NO: 101

SEQ ID NO: 104 - protein, 1205 aa, synthetic construct
MKLEDFTNLYSLSKTLRFELRPIGKTRENIENGGLLRQDEDRAEKYVHIKKLIDEYHKAYIDKQLSGLVLQYADIGKANSLEEYYHSTRKSKDSDKDKIVKIQDNLRKQIVKRLKDSDEFKRIDKKELIQSDLAEFIKPAEDRALIAEFKNFTTYFTGFNRNRQNMYSDKAISTAIAYRLIHENLPKFIDNIETFDRIAGITELYDQTSSDAEIFRLAHFSETLSQKQIDAYNSVMGRYNMLINEYNQTHKQSRLPKFKMLYKQILSDREHPSWLPEQFESDTAVLTAIRECYDDLRIPMANLKTLLEGLGNYDPSGIFLRNDQHLSQISKRLTGDRSSIERSVTEDLLTSRKLNKRKSRTTDEEESRKLFKQKGSLSIGYIADTAKIDVERYFAKLGAINTVTEQSENLFAKAENARTTADELLANDYPAGKRLVQSNDDIALVKNLLDALMELQWFVKPLLGTGDEAGKDERFYGEFTQIWEQLDRITPLYNMVRNYVTRKPYSTDKFKLNFESAALLGGWDKNREPDCLSVILRKDEQYYLGIINKNHKKIFARDILPCEGECYEKMVYKQISISAGIGGFFRKCYRTAEQYGWCCSDSCLNDERKIIIRDDEARENLTEIINCQKDFLNKYEKDGFKYRDFGFRFAASSDYEKLSDFYRDIDEQRYKLSFTPVSATYIDSLVEEGKLYLFQIYNKDFSAYSKGTPNMHTLYWRMLFDERNLSDVVYQLNGGAELFFRRKSLQNGRPTHPANIPIKNKNSRNDKKESLFDYDLIKDRRYTVDKFQFHVPITLNFKSDGAGRINERVREYLRSADDVHVIGIDRGERNLLYLVVTDMDGNICEQFSLNEICNTDYHSLLDEREHKRMQERQSWQAIEGIKELKEGYLSQVVHRIATLMVKYRAIVVLEDLNFGFMRSRQKVEKSVYQKFEHMLIDKLNYLVDKKANPTTPGGLLKAYQLTDKFESFQKLGKQSGFLFYVPAWNTSKIDPATGFVNMLDLGYESIDKAKALLCKFDSIRYNACKDWFEFALDYDKFGSKATGTRTKWTVCTYGQRIDTYRNKDSQWVSRDVDLTNELKSLFSEHGIDIYSNLKDAIVAQNDKEFFANMLRILKLTMQMRNSKTGTDIDYIVSPVADANGRFFDSRQADATMPKDADANGAYNIARKGIMLVQQIKQSDDLRTMKFDISNKSWLRFVQHTNQADE

SEQ ID NO: 105 - protein, 1205 aa, synthetic construct
MKLEDFTNLYSLSKTLRFELRPIGKTRENIENGGLLRQDEDRAEKYVHIKKLIDEYHKAYIDKQLSGLVLQYADIGKANSLEEYYHSTRKSKDSDKDKIVKIQDNLRKQIVKRLKDSDEFKRIDKKELIQSDLAEFIKPAEDRALIAEFKNFTTYFTGFNENRQNMYSDKAISTAIAYRLIHENLPKFIDNIETFDRIAGITELYDQTSSDAEIFRLAHFSETLSQKQIDAYNSVMGRYNMLINEYNQTHKQSRLPKFKMLYKQILSDREHPSWLPEQFESDTAVLTAIRECYDDLRIPMANLKTLLEGLGNYDPSGIFLRNDQHLSQISKRLTGDRSSIERSVTEDLLTSRKLNKRKSRTTDEEESRKLFKQKGSLSIGYIADTAKIDVERYFAKLGAINTVTEQSENLFAKAENARTTADELLANDYPAGKRLVQSNDDIALVKNLLDALMELQWFVKPLLGTGDEAGKDERFYGEFTQIWEQLDRITPLYNMVRNYVTRKPYSTDKFKLNFESAALLRGWDKNREPDCLSVILRKDEQYYLGIINKNHKKIFARDILPCEGECYEKMVYKQISISAGIGGFFRKCYRTAEQYGWCCSDSCLNDERKIIIRDDEARENLTEIINCQKDFLNKYEKDGFKYRDFGFRFAASSDYEKLSDFYRDIDEQRYKLSFTPVSATYIDSLVEEGKLYLFQIYNKDFSAYSKGTPNMHTLYWRMLFDERNLSDVVYQLNGGAELFFRRKSLQNGRPTHPANIPIKNKNSRNDKKESLFDYDLIKDRRYTVDKFQFHVPITLNFKSDGAGRINERVREYLRSADDVHVIGIDRGERNLLYLVVTDMDGNICEQFSLNEICNTDYHSLLDEREHKRMQERQSWQAIEGIKELKEGYLSQVVHRIATLMVKYRAIVVLEDLNFGFMRSRQKVEKSVYQKFEHMLIDKLNYLVDKKANPTTPGGLLKAYQLTDKFESFQKLGKQSGFLFYVPAWNTSKIDPATGFVNMLDLGYESIDKAKALLCKFDSIRYNACKDWFEFALDYDKFGSKATGTRTKWTVCTYGQRIDTYRNKDSQWVSRDVDLTNELKSLFSEHGIDIYSNLKDAIVAQNDKEFFANMLRILKLTMQMRNSKTGTDIDYIVSPVADANGRFFDSRQADATMPKDADANGAYNIARKGIMLVQQIKQSDDLRTMKFDISNKSWLRFVQHTNQADE

SEQ ID NO: 106 - protein, 1205 aa, synthetic construct
MKLEDFTNLYSLSKTLRFELRPIGKTRENIENGGLLRQDEDRAEKYVHIKKLIDEYHKAYIDKQLSGLVLQYADIGKANSLEEYYHSTRKSKDSDKDKIVKIQDNLRKQIVKRLKDSDEFKRIDKKELIQSDLAEFIKPAEDRALIAEFKNFTTYFTGFNRNRQNMYSDKAISTAIAYRLIHENLPKFIDNIETFDRIAGITELYDQTSSDAEIFRLAHFSETLSQKQIDAYNSVMGRYNMLINEYNQTHKQSRLPKFKMLYKQILSDREHPSWLPEQFESDTAVLTAIRECYDDLRIPMANLKTLLEGLGNYDPSGIFLRNDQHLSQISKRLTGDRSSIERSVTEDLLTSRKLNKRKSRTTDEEESRKLFKQKGSLSIGYIADTAKIDVERYFAKLGAINTVTEQSENLFAKAENARTTADELLANDYPAGKRLVQSNDDIALVKNLLDALMELQWFVKPLLGTGDEAGKDERFYGEFTQIWEQLDRITPLYNMVRNYVTRKPYSTDKFKLNFESAALLRGWDKNREPDCLSVILRKDEQYYLGIINKNHKKIFARDILPCEGECYEKMVYKQISISAGIGGFFRKCYRTAEQYGWCCSDSCLNDERKIIIRDDEARENLTEIINCQKDFLNKYEKDGFKYRDFGFRFAASSDYEKLSDFYRDIDEQRYKLSFTPVSATYIDSLVEEGKLYLFQIYNKDFSAYSKGTPNMHTLYWRMLFDERNLSDVVYQLNGGAELFFRRKSLQNGRPTHPANIPIKNKNSRNDKKESLFDYDLIKDRRYTVDKFQFHVPITLNFKSDGAGRINERVREYLRSADDVHVIGIDRGERNLLYLVVTDMDGNICEQFSLNEICNTDYHSLLDEREHKRMQERQSWQAIEGIKELKEGYLSQVVHRIATLMVKYRAIVVLEDLNFGFMRSRQKVEKSVYQKFEHMLIDKLNYLVDKKANPTTPGGLLKAYQLTDKFESFQKLGKQSGFLFYVPAWNTSKIDPATGFVNMLDLGYESIDKAKALLCKFDSIRYNACKDWFEFALDYDKFGSKATGTRTKWTVCTYGQRIDTYRNKDSQWVSRDVDLTNELKSLFSEHGIDIYSNLKDAIVAQNDKEFFANMLRILKLTMQMRNSKTGTDIDYIVSPVADANGRFFDSRQADATMPKDADANGAYNIARKGIMLVQQIKQSDDLRTMKFDISNKSWLRFVQHTNQADE

SEQ ID NO: 107 - protein, 1205 aa, synthetic construct
MKLEDFTNLYSLSKTLRFELRPIGKTRENIENGGLLRQDEDRAEKYVHIKKLIDEYHKAYIDKQLSGLVLQYADIGKANSLEEYYHSTRKSKDSDKDKIVKIQDNLRKQIVKRLKDSDEFKRIDKKELIQSDLAEFIKPAEDRALIAEFKNFTTYFTGFNRNRQNMYSDKAISTAIAYRLIHENLPKFIDNIETFDRIAGITELYDQTSSDAEIFRLAHFSETLSQKQIDAYNSVMGRYNMLIAEYNQTHKQSRLPKFKMLYKQILSDREHPSWLPEQFESDTAVLTAIRECYDDLRIPMANLKTLLEGLGNYDPSGIFLRNDQHLSQISKRLTGDRSSIERSVTEDLLTSRKLNKRKSRTTDEEESRKLFKQKGSLSIGYIADTAKIDVERYFAKLGAINTVTEQSENLFAKAENARTTADELLANDYPAGKRLVQSNDDIALVKNLLDALMELQWFVKPLLGTGDEAGKDERFYGEFTQIWEQLDRITPLYNMVRNYVTRKPYSTDKFKLNFESAALLGGWDKNREPDCLSVILRKDEQYYLGIINKNHKKIFARDILPCEGECYEKMVYKQISISAGIGGFFRKCYRTAEQYGWCCSDSCLNDERKIIIRDDEARENLTEIINCQKDFLNKYEKDGFKYRDFGFRFAASSDYEKLSDFYRDIDEQRYKLSFTPVSATYIDSLVEEGKLYLFQIYNKDFSAYSKGTPNMHTLYWRMLFDERNLSDVVYQLNGGAELFFRRKSLQNGRPTHPANIPIKNKNSRNDKKESLFDYDLIKDRRYTVDKFQFHVPITLNFKSDGAGRINERVREYLRSADDVHVIGIDRGERNLLYLVVTDMDGNICEQFSLNEICNTDYHSLLDEREHKRMQERQSWQAIEGIKELKEGYLSQVVHRIATLMVKYRAIVVLEDLNFGFMRSRQKVEKSVYQKFEHMLIDKLNYLVDKKANPTTPGGLLKAYQLTDKFESFQKLGKQSGFLFYVPAWNTSKIDPATGFVNMLDLGYESIDKAKALLCKFDSIRYNACKDWFEFALDYDKFGSKATGTRTKWTVCTYGQRIDTYRNKDSQWVSRDVDLTNELKSLFSEHGIDIYSNLKDAIVAQNDKEFFANMLRILKLTMQMRNSKTGTDIDYIVSPVADANGRFFDSRQADATMPKDADANGAYNIARKGIMLVQQIKQSDDLRTMKFDISNKSWLRFVQHTNQADE

SEQ ID NO: 108 - protein, 1205 aa, synthetic construct
MKLEDFTNLYSLSKTLRFELRPIGKTRENIENGGLLRQDEDRAEKYVHIKKLIDEYHKAYIDKQLSGLVLQYADIGKANSLEEYYHSTRKSKDSDKDKIVKIQDNLRKQIVKRLKDSDEFKRIDKKELIQSDLAEFIKPAEDRALIAEFKNFTTYFTGFNENRQNMYSDKAISTAIAYRLIHENLPKFIDNIETFDRIAGITELYDQTSSDAEIFRLAHFSETLSQKQIDAYNSVMGRYNMLINEYNQTHKQSRLPKFKMLYKQILSDREHPSWLPEQFESDTAVLTAIRECYDDLRIPMANLKTLLEGLGNYDPSGIFLRNDQHLSQISKRLTGDRSSIERSVTEDLLTSRKLNKRKSRTTDEEESRKLFKQKGSLSIGYIADTAKIDVERYFAKLGAINTVTEQSENLFAKAENARTTADELLANDYPAGKRLVQSNDDIALVKNLLDALMELQWFVKPLLGTGDEAGKDERFYGEFTQIWEQLDRITPLYNMVRNYVTRKPYSTDKFKLNFERAALLGGWDKNKEPDCLSVILRKDEQYYLGIINKNHKKIFARDILPCEGECYEKMVYKQISISAGIGGFFRKCYRTAEQYGWCCSDSCLNDERKIIIRDDEARENLTEIINCQKDFLNKYEKDGFKYRDFGFRFAASSDYEKLSDFYRDIDEQRYKLSFTPVSATYIDSLVEEGKLYLFQIYNKDFSAYSKGTPNMHTLYWRMLFDERNLSDVVYQLNGGAELFFRRKSLQNGRPTHPANIPIKNKNSRNDKKESLFDYDLIKDRRYTVDKFQFHVPITLNFKSDGAGRINERVREYLRSADDVHVIGIDRGERNLLYLVVTDMDGNICEQFSLNEICNTDYHSLLDEREHKRMQERQSWQAIEGIKELKEGYLSQVVHRIATLMVKYRAIVVLEDLNFGFMRSRQKVEKSVYQKFEHMLIDKLNYLVDKKANPTTPGGLLKAYQLTDKFESFQKLGKQSGFLFYVPAWNTSKIDPATGFVNMLDLGYESIDKAKALLCKFDSIRYNACKDWFEFALDYDKFGSKATGTRTKWTVCTYGQRIDTYRNKDSQWVSRDVDLTNELKSLFSEHGIDIYSNLKDAIVAQNDKEFFANMLRILKLTMQMRNSKTGTDIDYIVSPVADANGRFFDSRQADATMPKDADANGAYNIARKGIMLVQQIKQSDDLRTMKFDISNKSWLRFVQHTNQADE

SEQ ID NO: 109 - protein, 1224 aa, synthetic construct
MSKIEKFIGKYKISKTLRFRAVPVGKTQDNIEKKGILEKDKKRSEDYEKVKAYLDSLHRDFIENTLKKVKLNELNEYACLFFSGTKDDGDKKKMEKLEEKMRKTISNEFCNDEMYKKIFSEKILSENNEEDVSDIVSSYKGFFTSLNGYVRNRKNLYVSDAKPTSIAYRCINENLPKFLRNVECYKKVVQVIPKEQIEYMSNNLNLSPYRIEDCFNIDFFEFCLSQGGIDLYNTFIGGYSKKDGTKVQGINEIVNLYNQKNKKDKEKYKLPQFTPLFKQILSDRDTKSFSIEKLENIYEVVELVKKSYSDEMFDDIETVFSNLNYYDASGIYVKNGPAITHISMNLTKDWATIRNNWNYEYDEKHSTKKNKNIEKYEDTRNTMYKKIDSFTLEYISRLVGKDIDELVKYFENEVANFVMDIKKTYSKLTPLFDRCQKENFDISEDEVNDIKGYLDNVKLLESFMKSFTINGKENNIDYVFYGKFTDDYDKLHEFDHIYNKVRNYITTSRKPYKLDKYKLYFDNPQLLGGWDINKEKDYRTVMLTKDGKYYFAIIDKGEHPFDNIPKDYFDNNGYYKKIIYRQIPNAAKYLSSKQIVPQNPPEEVKRILDKKKADSKSLTEEEKNIFIDYIKSDFLKNYKLLFDKNNNPYFNFAFRESSTYESLNEFFEDVERQAYSVRYENLPADYIDNLVNEGKIYLFEIYSKDFSEYSKGTNNLHTMYFKALFDNDNLKNTVFKLSGNAELFIRPASIKKDELVIHPKNQLLQNKNPLNPKKQSIFDYDLVKDKRFFENQYMLHISIEINKNERDAKKIKNINEMVRKELKDSDDNYIIGIDRGERNLLYVCVINSAGKIVEQMSLNEIINEYNGIKHTVDYQGLLDKCEKERNAQRQSWKSIENIKELKDGYISQVVHKLCQLVEKYDAIIAMENLNGGFKRGRTKFEKQVYQKFENKLINKMEYMADKKRKTTENGGILRGYQLTNGCINNSYQNGFIFYVPAWLTSKIDPTTGFVDLLKPKYTNVEEAHLWINKFNSITYDKKLDMFAFNINYSQFPRADIDYRKIWTFYTNGYRIETFRNSEKNNEFDWKEVHLTSVIKKLLEEYQINYISGKNIIDDLIQIKDKPFWNSFIKYIRLTLQMRNSITGRTDVDYIISPVINNEGTFYDSRKDLDEITLPQDADANGAYNIARKALWIIEKLKESPDEELNKVKLAITQREWLEYAQINI

SEQ ID NO: 110 - protein, 1224 aa, synthetic construct
MSKIEKFIGKYKISKTLRFRAVPVGKTQDNIEKKGILEKDKKRSEDYEKVKAYLDSLHRDFIENTLKKVKLNELNEYACLFFSGTKDDGDKKKMEKLEEKMRKTISNEFCNDEMYKKIFSEKILSENNEEDVSDIVSSYKGFFTSLNGYVNNRKNLYVSDAKPTSIAYRCINENLPKFLRNVECYKKVVQVIPKEQIEYMSNNLNLSPYRIEDCFNIDFFEFCLSQGGIDLYNTFIGGYSKKDGTKVQGINEIVNLYNQKNKKDKEKYKLPQFTPLFKQILSDRDTKSFSIEKLENIYEVVELVKKSYSDEMFDDIETVFSNLNYYDASGIYVKNGPAITHISMNLTKDWATIRNNWNYEYDEKHSTKKNKNIEKYEDTRNTMYKKIDSFTLEYISRLVGKDIDELVKYFENEVANFVMDIKKTYSKLTPLFDRCQKENFDISEDEVNDIKGYLDNVKLLESFMKSFTINGKENNIDYVFYGKFTDDYDKLHEFDHIYNKVRNYITTSRKPYKLDKYKLYFDNPQLLRGWDINKEKDYRTVMLTKDGKYYFAIIDKGEHPFDNIPKDYFDNNGYYKKIIYRQIPNAAKYLSSKQIVPQNPPEEVKRILDKKKADSKSLTEEEKNIFIDYIKSDFLKNYKLLFDKNNNPYFNFAFRESSTYESLNEFFEDVERQAYSVRYENLPADYIDNLVNEGKIYLFEIYSKDFSEYSKGTNNLHTMYFKALFDNDNLKNTVFKLSGNAELFIRPASIKKDELVIHPKNQLLQNKNPLNPKKQSIFDYDLVKDKRFFENQYMLHISIEINKNERDAKKIKNINEMVRKELKDSDDNYIIGIDRGERNLLYVCVINSAGKIVEQMSLNEIINEYNGIKHTVDYQGLLDKCEKERNAQRQSWKSIENIKELKDGYISQVVHKLCQLVEKYDAIIAMENLNGGFKRGRTKFEKQVYQKFENKLINKMEYMADKKRKTTENGGILRGYQLTNGCINNSYQNGFIFYVPAWLTSKIDPTTGFVDLLKPKYTNVEEAHLWINKFNSITYDKKLDMFAFNINYSQFPRADIDYRKIWTFYTNGYRIETFRNSEKNNEFDWKEVHLTSVIKKLLEEYQINYISGKNIIDDLIQIKDKPFWNSFIKYIRLTLQMRNSITGRTDVDYIISPVINNEGTFYDSRKDLDEITLPQDADANGAYNIARKALWIIEKLKESPDEELNKVKLAITQREWLEYAQINI

SEQ ID NO: 111 - protein, 1224 aa, synthetic construct
MSKIEKFIGKYKISKTLRFRAVPVGKTQDNIEKKGILEKDKKRSEDYEKVKAYLDSLHRDFIENTLKKVKLNELNEYACLFFSGTKDDGDKKKMEKLEEKMRKTISNEFCNDEMYKKIFSEKILSENNEEDVSDIVSSYKGFFTSLNGYVNNRKNLYVSDAKPTSIAYRCINENLPKFLRNVECYKKVVQVIPKEQIEYMSNNLNLSPYRIEDCFNIDFFEFCLSQGGIDLYNTFIGGYSKKDGTKVQGINEIVNLYNQKNKKDKEKYKLPQFTPLFKQILSDRDTKSFSIEKLENIYEVVELVKKSYSDEMFDDIETVFSNLNYYDASGIYVKNGPAITHISMNLTKDWATIRNNWNYEYDEKHSTKKNKNIEKYEDTRNTMYKKIDSFTLEYISRLVGKDIDELVKYFENEVANFVMDIKKTYSKLTPLFDRCQKENFDISEDEVNDIKGYLDNVKLLESFMKSFTINGKENNIDYVFYGKFTDDYDKLHEFDHIYNKVRNYITTSRKPYKLDKYKLYFDNPQLLGGWDINREKDYRTVMLTKDGKYYFAIIDKGEHPFDNIPKDYFDNNGYYKKIIYRQIPNAAKYLSSKQIVPQNPPEEVKRILDKKKADSKSLTEEEKNIFIDYIKSDFLKNYKLLFDKNNNPYFNFAFRESSTYESLNEFFEDVERQAYSVRYENLPADYIDNLVNEGKIYLFEIYSKDFSEYSKGTNNLHTMYFKALFDNDNLKNTVFKLSGNAELFIRPASIKKDELVIHPKNQLLQNKNPLNPKKQSIFDYDLVKDKRFFENQYMLHISIEINKNERDAKKIKNINEMVRKELKDSDDNYIIGIDRGERNLLYVCVINSAGKIVEQMSLNEIINEYNGIKHTVDYQGLLDKCEKERNAQRQSWKSIENIKELKDGYISQVVHKLCQLVEKYDAIIAMENLNGGFKRGRTKFEKQVYQKFENKLINKMEYMADKKRKTTENGGILRGYQLTNGCINNSYQNGFIFYVPAWLTSKIDPTTGFVDLLKPKYTNVEEAHLWINKFNSITYDKKLDMFAFNINYSQFPRADIDYRKIWTFYTNGYRIETFRNSEKNNEFDWKEVHLTSVIKKLLEEYQINYISGKNIIDDLIQIKDKPFWNSFIKYIRLTLQMRNSITGRTDVDYIISPVINNEGTFYDSRKDLDEITLPQDADANGAYNIARKALWIIEKLKESPDEELNKVKLAITQREWLEYAQINI

SEQ ID NO: 112 - protein, 1224 aa, synthetic construct
MSKIEKFIGKYKISKTLRFRAVPVGKTQDNIEKKGILEKDKKRSEDYEKVKAYLDSLHRDFIENTLKKVKLNELNEYACLFFSGTKDDGDKKKMEKLEEKMRKTISNEFCNDEMYKKIFSEKILSENNEEDVSDIVSSYKGFFTSLNGYVRNRKNLYVSDAKPTSIAYRCINENLPKFLRNVECYKKVVQVIPKEQIEYMSNNLNLSPYRIEDCFNIDFFEFCLSQGGIDLYNTFIGGYSKKDGTKVQGINEIVNLYNQKNKKDKEKYKLPQFTPLFKQILSDRDTKSFSIEKLENIYEVVELVKKSYSDEMFDDIETVFSNLNYYDASGIYVKNGPAITHISMNLTKDWATIRNNWNYEYDEKHSTKKNKNIEKYEDTRNTMYKKIDSFTLEYISRLVGKDIDELVKYFENEVANFVMDIKKTYSKLTPLFDRCQKENFDISEDEVNDIKGYLDNVKLLESFMKSFTINGKENNIDYVFYGKFTDDYDKLHEFDHIYNKVRNYITTSRKPYKLDKYKLYFDNPQLLRGWDINKEKDYRTVMLTKDGKYYFAIIDKGEHPFDNIPKDYFDNNGYYKKIIYRQIPNAAKYLSSKQIVPQNPPEEVKRILDKKKADSKSLTEEEKNIFIDYIKSDFLKNYKLLFDKNNNPYFNFAFRESSTYESLNEFFEDVERQAYSVRYENLPADYIDNLVNEGKIYLFEIYSKDFSEYSKGTNNLHTMYFKALFDNDNLKNTVFKLSGNAELFIRPASIKKDELVIHPKNQLLQNKNPLNPKKQSIFDYDLVKDKRFFENQYMLHISIEINKNERDAKKIKNINEMVRKELKDSDDNYIIGIDRGERNLLYVCVINSAGKIVEQMSLNEIINEYNGIKHTVDYQGLLDKCEKERNAQRQSWKSIENIKELKDGYISQVVHKLCQLVEKYDAIIAMENLNGGFKRGRTKFEKQVYQKFENKLINKMEYMADKKRKTTENGGILRGYQLTNGCINNSYQNGFIFYVPAWLTSKIDPTTGFVDLLKPKYTNVEEAHLWINKFNSITYDKKLDMFAFNINYSQFPRADIDYRKIWTFYTNGYRIETFRNSEKNNEFDWKEVHLTSVIKKLLEEYQINYISGKNIIDDLIQIKDKPFWNSFIKYIRLTLQMRNSITGRTDVDYIISPVINNEGTFYDSRKDLDEITLPQDADANGAYNIARKALWIIEKLKESPDEELNKVKLAITQREWLEYAQINI

SEQ ID NO: 113 - protein, 1224 aa, synthetic construct
MSKIEKFIGKYKISKTLRFRAVPVGKTQDNIEKKGILEKDKKRSEDYEKVKAYLDSLHRDFIENTLKKVKLNELNEYACLFFSGTKDDGDKKKMEKLEEKMRKTISNEFCNDEMYKKIFSEKILSENNEEDVSDIVSSYKGFFTSLNGYVRNRKNLYVSDAKPTSIAYRCINENLPKFLRNVECYKKVVQVIPKEQIEYMSNNLNLSPYRIEDCFNIDFFEFCLSQGGIDLYNTFIGGYSKKDGTKVQGINEIVNLYNQKNKKDKEKYKLPQFTPLFKQILSDRDTKSFSIEKLENIYEVVELVKKSYSDEMFDDIETVFSNLNYYDASGIYVKNGPAITHISMNLTKDWATIRNNWNYEYDEKHSTKKNKNIEKYEDTRNTMYKKIDSFTLEYISRLVGKDIDELVKYFENEVANFVMDIKKTYSKLTPLFDRCQKENFDISEDEVNDIKGYLDNVKLLESFMKSFTINGKENNIDYVFYGKFTDDYDKLHEFDHIYNKVRNYITTSRKPYKLDKYKLYFDNPQLLGGWDINREKDYRTVMLTKDGKYYFAIIDKGEHPFDNIPKDYFDNNGYYKKIIYRQIPNAAKYLSSKQIVPQNPPEEVKRILDKKKADSKSLTEEEKNIFIDYIKSDFLKNYKLLFDKNNNPYFNFAFRESSTYESLNEFFEDVERQAYSVRYENLPADYIDNLVNEGKIYLFEIYSKDFSEYSKGTNNLHTMYFKALFDNDNLKNTVFKLSGNAELFIRPASIKKDELVIHPKNQLLQNKNPLNPKKQSIFDYDLVKDKRFFENQYMLHISIEINKNERDAKKIKNINEMVRKELKDSDDNYIIGIDRGERNLLYVCVINSAGKIVEQMSLNEIINEYNGIKHTVDYQGLLDKCEKERNAQRQSWKSIENIKELKDGYISQVVHKLCQLVEKYDAIIAMENLNGGFKRGRTKFEKQVYQKFENKLINKMEYMADKKRKTTENGGILRGYQLTNGCINNSYQNGFIFYVPAWLTSKIDPTTGFVDLLKPKYTNVEEAHLWINKFNSITYDKKLDMFAFNINYSQFPRADIDYRKIWTFYTNGYRIETFRNSEKNNEFDWKEVHLTSVIKKLLEEYQINYISGKNIIDDLIQIKDKPFWNSFIKYIRLTLQMRNSITGRTDVDYIISPVINNEGTFYDSRKDLDEITLPQDADANGAYNIARKALWIIEKLKESPDEELNKVKLAITQREWLEYAQINI

SEQ ID NO: 114 - protein, 1224 aa, synthetic construct
MSKIEKFIGKYKISKTLRFRAVPVGKTQDNIEKKGILEKDKKRSEDYEKVKAYLDSLHRDFIENTLKKVKLNELNEYACLFFSGTKDDGDKKKMEKLEEKMRKTISNEFCNDEMYKKIFSEKILSENNEEDVSDIVSSYKGFFTSLNGYVNNRKNLYVSDAKPTSIAYRCINENLPKFLRNVECYKKVVQVIPKEQIEYMSNNLNLSPYRIEDCFNIDFFEFCLSQGGIDLYNTFIGGYSKKDGTKVQGINEIVNLYNQKNKKDKEKYKLPQFTPLFKQILSDRDTKSFSIEKLENIYEVVELVKKSYSDEMFDDIETVFSNLNYYDASGIYVKNGPAITHISMNLTKDWATIRNNWNYEYDEKHSTKKNKNIEKYEDTRNTMYKKIDSFTLEYISRLVGKDIDELVKYFENEVANFVMDIKKTYSKLTPLFDRCQKENFDISEDEVNDIKGYLDNVKLLESFMKSFTINGKENNIDYVFYGKFTDDYDKLHEFDHIYNKVRNYITTSRKPYKLDKYKLYFDNPQLLRGWDINREKDYRTVMLTKDGKYYFAIIDKGEHPFDNIPKDYFDNNGYYKKIIYRQIPNAAKYLSSKQIVPQNPPEEVKRILDKKKADSKSLTEEEKNIFIDYIKSDFLKNYKLLFDKNNNPYFNFAFRESSTYESLNEFFEDVERQAYSVRYENLPADYIDNLVNEGKIYLFEIYSKDFSEYSKGTNNLHTMYFKALFDNDNLKNTVFKLSGNAELFIRPASIKKDELVIHPKNQLLQNKNPLNPKKQSIFDYDLVKDKRFFENQYMLHISIEINKNERDAKKIKNINEMVRKELKDSDDNYIIGIDRGERNLLYVCVINSAGKIVEQMSLNEIINEYNGIKHTVDYQGLLDKCEKERNAQRQSWKSIENIKELKDGYISQVVHKLCQLVEKYDAIIAMENLNGGFKRGRTKFEKQVYQKFENKLINKMEYMADKKRKTTENGGILRGYQLTNGCINNSYQNGFIFYVPAWLTSKIDPTTGFVDLLKPKYTNVEEAHLWINKFNSITYDKKLDMFAFNINYSQFPRADIDYRKIWTFYTNGYRIETFRNSEKNNEFDWKEVHLTSVIKKLLEEYQINYISGKNIIDDLIQIKDKPFWNSFIKYIRLTLQMRNSITGRTDVDYIISPVINNEGTFYDSRKDLDEITLPQDADANGAYNIARKALWIIEKLKESPDEELNKVKLAITQREWLEYAQINI

SEQ ID NO: 115 - protein, 1224 aa, synthetic construct
MSKIEKFIGKYKISKTLRFRAVPVGKTQDNIEKKGILEKDKKRSEDYEKVKAYLDSLHRDFIENTLKKVKLNELNEYACLFFSGTKDDGDKKKMEKLEEKMRKTISNEFCNDEMYKKIFSEKILSENNEEDVSDIVSSYKGFFTSLNGYVRNRKNLYVSDAKPTSIAYRCINENLPKFLRNVECYKKVVQVIPKEQIEYMSNNLNLSPYRIEDCFNIDFFEFCLSQGGIDLYNTFIGGYSKKDGTKVQGINEIVNLYNQKNKKDKEKYKLPQFTPLFKQILSDRDTKSFSIEKLENIYEVVELVKKSYSDEMFDDIETVFSNLNYYDASGIYVKNGPAITHISMNLTKDWATIRNNWNYEYDEKHSTKKNKNIEKYEDTRNTMYKKIDSFTLEYISRLVGKDIDELVKYFENEVANFVMDIKKTYSKLTPLFDRCQKENFDISEDEVNDIKGYLDNVKLLESFMKSFTINGKENNIDYVFYGKFTDDYDKLHEFDHIYNKVRNYITTSRKPYKLDKYKLYFDNPQLLRGWDINREKDYRTVMLTKDGKYYFAIIDKGEHPFDNIPKDYFDNNGYYKKIIYRQIPNAAKYLSSKQIVPQNPPEEVKRILDKKKADSKSLTEEEKNIFIDYIKSDFLKNYKLLFDKNNNPYFNFAFRESSTYESLNEFFEDVERQAYSVRYENLPADYIDNLVNEGKIYLFEIYSKDFSEYSKGTNNLHTMYFKALFDNDNLKNTVFKLSGNAELFIRPASIKKDELVIHPKNQLLQNKNPLNPKKQSIFDYDLVKDKRFFENQYMLHISIEINKNERDAKKIKNINEMVRKELKDSDDNYIIGIDRGERNLLYVCVINSAGKIVEQMSLNEIINEYNGIKHTVDYQGLLDKCEKERNAQRQSWKSIENIKELKDGYISQVVHKLCQLVEKYDAIIAMENLNGGFKRGRTKFEKQVYQKFENKLINKMEYMADKKRKTTENGGILRGYQLTNGCINNSYQNGFIFYVPAWLTSKIDPTTGFVDLLKPKYTNVEEAHLWINKFNSITYDKKLDMFAFNINYSQFPRADIDYRKIWTFYTNGYRIETFRNSEKNNEFDWKEVHLTSVIKKLLEEYQINYISGKNIIDDLIQIKDKPFWNSFIKYIRLTLQMRNSITGRTDVDYIISPVINNEGTFYDSRKDLDEITLPQDADANGAYNIARKALWIIEKLKESPDEELNKVKLAITQREWLEYAQINI

SEQ ID NO: 116 - protein, 1224 aa, synthetic construct
MSKIEKFIGKYKISKTLRFRAVPVGKTQDNIEKKGILEKDKKRSEDYEKVKAYLDSLHRDFIENTLKKVKLNELNEYACLFFSGTKDDGDKKKMEKLEEKMRKTISNEFCNDEMYKKIFSEKILSENNEEDVSDIVSSYKGFFTSLNGYVRNRKNLYVSDAKPTSIAYRCINENLPKFLRNVECYKKVVQVIPKEQIEYMSNNLNLSPYRIEDCFNIDFFEFCLSQGGIDLYNTFIGGYSKKDGTKVQGINEIVALYNQKNKKDKEKYKLPQFTPLFKQILSDRDTKSFSIEKLENIYEVVELVKKSYSDEMFDDIETVFSNLNYYDASGIYVKNGPAITHISMNLTKDWATIRNNWNYEYDEKHSTKKNKNIEKYEDTRNTMYKKIDSFTLEYISRLVGKDIDELVKYFENEVANFVMDIKKTYSKLTPLFDRCQKENFDISEDEVNDIKGYLDNVKLLESFMKSFTINGKENNIDYVFYGKFTDDYDKLHEFDHIYNKVRNYITTSRKPYKLDKYKLYFDNPQLLGGWDINREKDYRTVMLTKDGKYYFAIIDKGEHPFDNIPKDYFDNNGYYKKIIYRQIPNAAKYLSSKQIVPQNPPEEVKRILDKKKADSKSLTEEEKNIFIDYIKSDFLKNYKLLFDKNNNPYFNFAFRESSTYESLNEFFEDVERQAYSVRYENLPADYIDNLVNEGKIYLFEIYSKDFSEYSKGTNNLHTMYFKALFDNDNLKNTVFKLSGNAELFIRPASIKKDELVIHPKNQLLQNKNPLNPKKQSIFDYDLVKDKRFFENQYMLHISIEINKNERDAKKIKNINEMVRKELKDSDDNYIIGIDRGERNLLYVCVINSAGKIVEQMSLNEIINEYNGIKHTVDYQGLLDKCEKERNAQRQSWKSIENIKELKDGYISQVVHKLCQLVEKYDAIIAMENLNGGFKRGRTKFEKQVYQKFENKLINKMEYMADKKRKTTENGGILRGYQLTNGCINNSYQNGFIFYVPAWLTSKIDPTTGFVDLLKPKYTNVEEAHLWINKFNSITYDKKLDMFAFNINYSQFPRADIDYRKIWTFYTNGYRIETFRNSEKNNEFDWKEVHLTSVIKKLLEEYQINYISGKNIIDDLIQIKDKPFWNSFIKYIRLTLQMRNSITGRTDVDYIISPVINNEGTFYDSRKDLDEITLPQDADANGAYNIARKALWIIEKLKESPDEELNKVKLAITQREWLEYAQINI

SEQ ID NO: 117 - protein, 1224 aa, synthetic construct
MSKIEKFIGKYKISKTLRFRAVPVGKTQDNIEKKGILEKDKKRSEDYEKVKAYLDSLHRDFIENTLKKVKLNELNEYACLFFSGTKDDGDKKKMEKLEEKMRKTISNEFCNDEMYKKIFSEKILSENNEEDVSDIVSSYKGFFTSLNGYVNNRKNLYVSDAKPTSIAYRCINENLPKFLRNVECYKKVVQVIPKEQIEYMSNNLNLSPYRIEDCFNIDFFEFCLSQGGIDLYNTFIGGYSKKDGTKVQGINEIVNLYNQKNKKDKEKYKLPQFTPLFKQILSDRDTKSFSIEKLENIYEVVELVKKSYSDEMFDDIETVFSNLNYYDASGIYVKNGPAITHISMNLTKDWATIRNNWNYEYDEKHSTKKNKNIEKYEDTRNTMYKKIDSFTLEYISRLVGKDIDELVKYFENEVANFVMDIKKTYSKLTPLFDRCQKENFDISEDEVNDIKGYLDNVKLLESFMKSFTINGKENNIDYVFYGKFTDDYDKLHEFDHIYNKVRNYITTSRKPYKLDKYKLYFDRPQLLGGWDINKEKDYRTVMLTKDGKYYFAIIDKGEHPFDNIPKDYFDNNGYYKKIIYRQIPNAAKYLSSKQIVPQNPPEEVKRILDKKKADSKSLTEEEKNIFIDYIKSDFLKNYKLLFDKNNNPYFNFAFRESSTYESLNEFFEDVERQAYSVRYENLPADYIDNLVNEGKIYLFEIYSKDFSEYSKGTNNLHTMYFKALFDNDNLKNTVFKLSGNAELFIRPASIKKDELVIHPKNQLLQNKNPLNPKKQSIFDYDLVKDKRFFENQYMLHISIEINKNERDAKKIKNINEMVRKELKDSDDNYIIGIDRGERNLLYVCVINSAGKIVEQMSLNEIINEYNGIKHTVDYQGLLDKCEKERNAQRQSWKSIENIKELKDGYISQVVHKLCQLVEKYDAIIAMENLNGGFKRGRTKFEKQVYQKFENKLINKMEYMADKKRKTTENGGILRGYQLTNGCINNSYQNGFIFYVPAWLTSKIDPTTGFVDLLKPKYTNVEEAHLWINKFNSITYDKKLDMFAFNINYSQFPRADIDYRKIWTFYTNGYRIETFRNSEKNNEFDWKEVHLTSVIKKLLEEYQINYISGKNIIDDLIQIKDKPFWNSFIKYIRLTLQMRNSITGRTDVDYIISPVINNEGTFYDSRKDLDEITLPQDADANGAYNIARKALWIIEKLKESPDEELNKVKLAITQREWLEYAQINI

SEQ ID NO: 118 - protein, 1226 aa, synthetic construct
MKNFFQLFTNQYELSKTLRFELRPIENTAELLKKNKIFEKDKKVYEYYQKTKKYFDKLHCEFIDEALVNTSLPNKDYSIYDKLFFEYKKDKENKKIYKALNEHAKKLRTNIVKDFEKTAEKWRDEYILSIKDEKSQKKLKQLEGLDLLFKTEVFDFLKKKYPEAQIEGNSIFDSFNKFFTYFDNFHRSRKNFYKDDGTASAIPTRIIDDNLPKFLENKKIFEEEYKVLNLSREEKDLFRLDFYNKCFTQTGIENYNEIVSKINSKVNKYRQKNKKSKIHFMKELFKQILSKRSKQATEQDTSISIENDSQVFEVLQDFIMETKNCNRMAKNILETFIQHQEEFDLQKVFLKGSNVTRISDMWFTSWFAFGDLLPKNSTGKKLANFISFQDIKNALEKTEIQDVFKKKYAKYNKESFYDQFLQIFDYEFHKALNDCEKNIVEAEKMMKVDKVYSNKKGILRNGKNGEKQKEIIKDFADSALSIYQMMKYFALEKGKKPVTDMDEDPRFYNDYKDWCEKSRTLLYYNEFRNYLTKKPYAEDKIKLNFEKSTLLMGWSANEEGNLQYYSSILRKDNKYYLGLMSSASTFNINKKEAFETRDGFYELMNYRQLKSTTIYGSLYKGEYKKEYRKDKETLTNQELIERTKKILEKQKFSYPQLEVILDKKYKAAEDLAKDLGNIVLYNIGFVKISKRYVNNLTNENFYLFEISNKDFLNKKKKGNVNLHTKYFQLLFDKKNLESKKGAILKLSGGGEVFYRKATKNLPKKKDKKGKEIIDRKRYAEDKILFHLPIVLNIGFKDKQINSKVNQVLATQRKARIIGIDRGEKHLLYISVIDENANILKIKSLNTIDVPNKKEPDDYHKLLDEKEKERDDERKSWHTIENIKELKHGYISQVVNEIDKIIFECLDERILPIIVFEDLNIGFKRGRFKIEKQVYQKFELALAKKLNYLVSKEKGNYLNAFQFTPPVNNFQDIHKKQVGIIFYIPASFTSAICPVCGFRKRLYGFTFKNINQVKKLLAEHEFNIFYDGKRFNFECLASKENEKKENKNGLYTEIFLNKKLFFNSDIERLFNKKSKDNKKWETISFIATEELKKLFEGKIDLKRNIMDQILAGDFRAEFYQQLIFIINRILNLRNCHAVEHRDFIACPSCGFHSEKNYEKLKSRYAGKEKFEFNGDANGAYNIARKGILLIKKIRQFAKSNNIEKLMPYDHLQIDMQEWDKFCAKNKS

SEQ ID NO: 119 - protein, 1226 aa, synthetic construct
MKNFFQLFTNQYELSKTLRFELRPIENTAELLKKNKIFEKDKKVYEYYQKTKKYFDKLHCEFIDEALVNTSLPNKDYSIYDKLFFEYKKDKENKKIYKALNEHAKKLRTNIVKDFEKTAEKWRDEYILSIKDEKSQKKLKQLEGLDLLFKTEVFDFLKKKYPEAQIEGNSIFDSFNKFFTYFDNFHQSRKNFYKDDGTASAIPTRIIDDNLPKFLENKKIFEEEYKVLNLSREEKDLFRLDFYNKCFTQTGIENYNEIVSKINSKVNKYRQKNKKSKIHFMKELFKQILSKRSKQATEQDTSISIENDSQVFEVLQDFIMETKNCNRMAKNILETFIQHQEEFDLQKVFLKGSNVTRISDMWFTSWFAFGDLLPKNSTGKKLANFISFQDIKNALEKTEIQDVFKKKYAKYNKESFYDQFLQIFDYEFHKALNDCEKNIVEAEKMMKVDKVYSNKKGILRNGKNGEKQKEIIKDFADSALSIYQMMKYFALEKGKKPVTDMDEDPRFYNDYKDWCEKSRTLLYYNEFRNYLTKKPYAEDKIKLNFEKSTLLRGWSANEEGNLQYYSSILRKDNKYYLGLMSSASTFNINKKEAFETRDGFYELMNYRQLKSTTIYGSLYKGEYKKEYRKDKETLTNQELIERTKKILEKQKFSYPQLEVILDKKYKAAEDLAKDLGNIVLYNIGFVKISKRYVNNLTNENFYLFEISNKDFLNKKKKGNVNLHTKYFQLLFDKKNLESKKGAILKLSGGGEVFYRKATKNLPKKKDKKGKEIIDRKRYAEDKILFHLPIVLNIGFKDKQINSKVNQVLATQRKARIIGIDRGEKHLLYISVIDENANILKIKSLNTIDVPNKKEPDDYHKLLDEKEKERDDERKSWHTIENIKELKHGYISQVVNEIDKIIFECLDERILPIIVFEDLNIGFKRGRFKIEKQVYQKFELALAKKLNYLVSKEKGNYLNAFQFTPPVNNFQDIHKKQVGIIFYIPASFTSAICPVCGFRKRLYGFTFKNINQVKKLLAEHEFNIFYDGKRFNFECLASKENEKKENKNGLYTEIFLNKKLFFNSDIERLFNKKSKDNKKWETISFIATEELKKLFEGKIDLKRNIMDQILAGDFRAEFYQQLIFIINRILNLRNCHAVEHRDFIACPSCGFHSEKNYEKLKSRYAGKEKFEFNGDANGAYNIARKGILLIKKIRQFAKSNNIEKLMPYDHLQIDMQEWDKFCAKNKS

SEQ ID NO: 120 - protein, 1226 aa, synthetic construct
MKNFFQLFTNQYELSKTLRFELRPIENTAELLKKNKIFEKDKKVYEYYQKTKKYFDKLHCEFIDEALVNTSLPNKDYSIYDKLFFEYKKDKENKKIYKALNEHAKKLRTNIVKDFEKTAEKWRDEYILSIKDEKSQKKLKQLEGLDLLFKTEVFDFLKKKYPEAQIEGNSIFDSFNKFFTYFDNFHQSRKNFYKDDGTASAIPTRIIDDNLPKFLENKKIFEEEYKVLNLSREEKDLFRLDFYNKCFTQTGIENYNEIVSKINSKVNKYRQKNKKSKIHFMKELFKQILSKRSKQATEQDTSISIENDSQVFEVLQDFIMETKNCNRMAKNILETFIQHQEEFDLQKVFLKGSNVTRISDMWFTSWFAFGDLLPKNSTGKKLANFISFQDIKNALEKTEIQDVFKKKYAKYNKESFYDQFLQIFDYEFHKALNDCEKNIVEAEKMMKVDKVYSNKKGILRNGKNGEKQKEIIKDFADSALSIYQMMKYFALEKGKKPVTDMDEDPRFYNDYKDWCEKSRTLLYYNEFRNYLTKKPYAEDKIKLNFEKSTLLMGWSANREGNLQYYSSILRKDNKYYLGLMSSASTFNINKKEAFETRDGFYELMNYRQLKSTTIYGSLYKGEYKKEYRKDKETLTNQELIERTKKILEKQKFSYPQLEVILDKKYKAAEDLAKDLGNIVLYNIGFVKISKRYVNNLTNENFYLFEISNKDFLNKKKKGNVNLHTKYFQLLFDKKNLESKKGAILKLSGGGEVFYRKATKNLPKKKDKKGKEIIDRKRYAEDKILFHLPIVLNIGFKDKQINSKVNQVLATQRKARIIGIDRGEKHLLYISVIDENANILKIKSLNTIDVPNKKEPDDYHKLLDEKEKERDDERKSWHTIENIKELKHGYISQVVNEIDKIIFECLDERILPIIVFEDLNIGFKRGRFKIEKQVYQKFELALAKKLNYLVSKEKGNYLNAFQFTPPVNNFQDIHKKQVGIIFYIPASFTSAICPVCGFRKRLYGFTFKNINQVKKLLAEHEFNIFYDGKRFNFECLASKENEKKENKNGLYTEIFLNKKLFFNSDIERLFNKKSKDNKKWETISFIATEELKKLFEGKIDLKRNIMDQILAGDFRAEFYQQLIFIINRILNLRNCHAVEHRDFIACPSCGFHSEKNYEKLKSRYAGKEKFEFNGDANGAYNIARKGILLIKKIRQFAKSNNIEKLMPYDHLQIDMQEWDKFCAKNKS

SEQ ID NO: 121 - protein, 1226 aa, synthetic construct
MKNFFQLFTNQYELSKTLRFELRPIENTAELLKKNKIFEKDKKVYEYYQKTKKYFDKLHCEFIDEALVNTSLPNKDYSIYDKLFFEYKKDKENKKIYKALNEHAKKLRTNIVKDFEKTAEKWRDEYILSIKDEKSQKKLKQLEGLDLLFKTEVFDFLKKKYPEAQIEGNSIFDSFNKFFTYFDNFHRSRKNFYKDDGTASAIPTRIIDDNLPKFLENKKIFEEEYKVLNLSREEKDLFRLDFYNKCFTQTGIENYNEIVSKINSKVNKYRQKNKKSKIHFMKELFKQILSKRSKQATEQDTSISIENDSQVFEVLQDFIMETKNCNRMAKNILETFIQHQEEFDLQKVFLKGSNVTRISDMWFTSWFAFGDLLPKNSTGKKLANFISFQDIKNALEKTEIQDVFKKKYAKYNKESFYDQFLQIFDYEFHKALNDCEKNIVEAEKMMKVDKVYSNKKGILRNGKNGEKQKEIIKDFADSALSIYQMMKYFALEKGKKPVTDMDEDPRFYNDYKDWCEKSRTLLYYNEFRNYLTKKPYAEDKIKLNFEKSTLLRGWSANEEGNLQYYSSILRKDNKYYLGLMSSASTFNINKKEAFETRDGFYELMNYRQLKSTTIYGSLYKGEYKKEYRKDKETLTNQELIERTKKILEKQKFSYPQLEVILDKKYKAAEDLAKDLGNIVLYNIGFVKISKRYVNNLTNENFYLFEISNKDFLNKKKKGNVNLHTKYFQLLFDKKNLESKKGAILKLSGGGEVFYRKATKNLPKKKDKKGKEIIDRKRYAEDKILFHLPIVLNIGFKDKQINSKVNQVLATQRKARIIGIDRGEKHLLYISVIDENANILKIKSLNTIDVPNKKEPDDYHKLLDEKEKERDDERKSWHTIENIKELKHGYISQVVNEIDKIIFECLDERILPIIVFEDLNIGFKRGRFKIEKQVYQKFELALAKKLNYLVSKEKGNYLNAFQFTPPVNNFQDIHKKQVGIIFYIPASFTSAICPVCGFRKRLYGFTFKNINQVKKLLAEHEFNIFYDGKRFNFECLASKENEKKENKNGLYTEIFLNKKLFFNSDIERLFNKKSKDNKKWETISFIATEELKKLFEGKIDLKRNIMDQILAGDFRAEFYQQLIFIINRILNLRNCHAVEHRDFIACPSCGFHSEKNYEKLKSRYAGKEKFEFNGDANGAYNIARKGILLIKKIRQFAKSNNIEKLMPYDHLQIDMQEWDKFCAKNKS

SEQ ID NO: 122 - protein, 1226 aa, synthetic construct
MKNFFQLFTNQYELSKTLRFELRPIENTAELLKKNKIFEKDKKVYEYYQKTKKYFDKLHCEFIDEALVNTSLPNKDYSIYDKLFFEYKKDKENKKIYKALNEHAKKLRTNIVKDFEKTAEKWRDEYILSIKDEKSQKKLKQLEGLDLLFKTEVFDFLKKKYPEAQIEGNSIFDSFNKFFTYFDNFHRSRKNFYKDDGTASAIPTRIIDDNLPKFLENKKIFEEEYKVLNLSREEKDLFRLDFYNKCFTQTGIENYNEIVSKINSKVNKYRQKNKKSKIHFMKELFKQILSKRSKQATEQDTSISIENDSQVFEVLQDFIMETKNCNRMAKNILETFIQHQEEFDLQKVFLKGSNVTRISDMWFTSWFAFGDLLPKNSTGKKLANFISFQDIKNALEKTEIQDVFKKKYAKYNKESFYDQFLQIFDYEFHKALNDCEKNIVEAEKMMKVDKVYSNKKGILRNGKNGEKQKEIIKDFADSALSIYQMMKYFALEKGKKPVTDMDEDPRFYNDYKDWCEKSRTLLYYNEFRNYLTKKPYAEDKIKLNFEKSTLLMGWSANREGNLQYYSSILRKDNKYYLGLMSSASTFNINKKEAFETRDGFYELMNYRQLKSTTIYGSLYKGEYKKEYRKDKETLTNQELIERTKKILEKQKFSYPQLEVILDKKYKAAEDLAKDLGNIVLYNIGFVKISKRYVNNLTNENFYLFEISNKDFLNKKKKGNVNLHTKYFQLLFDKKNLESKKGAILKLSGGGEVFYRKATKNLPKKKDKKGKEIIDRKRYAEDKILFHLPIVLNIGFKDKQINSKVNQVLATQRKARIIGIDRGEKHLLYISVIDENANILKIKSLNTIDVPNKKEPDDYHKLLDEKEKERDDERKSWHTIENIKELKHGYISQVVNEIDKIIFECLDERILPIIVFEDLNIGFKRGRFKIEKQVYQKFELALAKKLNYLVSKEKGNYLNAFQFTPPVNNFQDIHKKQVGIIFYIPASFTSAICPVCGFRKRLYGFTFKNINQVKKLLAEHEFNIFYDGKRFNFECLASKENEKKENKNGLYTEIFLNKKLFFNSDIERLFNKKSKDNKKWETISFIATEELKKLFEGKIDLKRNIMDQILAGDFRAEFYQQLIFIINRILNLRNCHAVEHRDFIACPSCGFHSEKNYEKLKSRYAGKEKFEFNGDANGAYNIARKGILLIKKIRQFAKSNNIEKLMPYDHLQIDMQEWDKFCAKNKS

SEQ ID NO: 123 - protein, 1226 aa, synthetic construct
MKNFFQLFTNQYELSKTLRFELRPIENTAELLKKNKIFEKDKKVYEYYQKTKKYFDKLHCEFIDEALVNTSLPNKDYSIYDKLFFEYKKDKENKKIYKALNEHAKKLRTNIVKDFEKTAEKWRDEYILSIKDEKSQKKLKQLEGLDLLFKTEVFDFLKKKYPEAQIEGNSIFDSFNKFFTYFDNFHQSRKNFYKDDGTASAIPTRIIDDNLPKFLENKKIFEEEYKVLNLSREEKDLFRLDFYNKCFTQTGIENYNEIVSKINSKVNKYRQKNKKSKIHFMKELFKQILSKRSKQATEQDTSISIENDSQVFEVLQDFIMETKNCNRMAKNILETFIQHQEEFDLQKVFLKGSNVTRISDMWFTSWFAFGDLLPKNSTGKKLANFISFQDIKNALEKTEIQDVFKKKYAKYNKESFYDQFLQIFDYEFHKALNDCEKNIVEAEKMMKVDKVYSNKKGILRNGKNGEKQKEIIKDFADSALSIYQMMKYFALEKGKKPVTDMDEDPRFYNDYKDWCEKSRTLLYYNEFRNYLTKKPYAEDKIKLNFEKSTLLRGWSANREGNLQYYSSILRKDNKYYLGLMSSASTFNINKKEAFETRDGFYELMNYRQLKSTTIYGSLYKGEYKKEYRKDKETLTNQELIERTKKILEKQKFSYPQLEVILDKKYKAAEDLAKDLGNIVLYNIGFVKISKRYVNNLTNENFYLFEISNKDFLNKKKKGNVNLHTKYFQLLFDKKNLESKKGAILKLSGGGEVFYRKATKNLPKKKDKKGKEIIDRKRYAEDKILFHLPIVLNIGFKDKQINSKVNQVLATQRKARIIGIDRGEKHLLYISVIDENANILKIKSLNTIDVPNKKEPDDYHKLLDEKEKERDDERKSWHTIENIKELKHGYISQVVNEIDKIIFECLDERILPIIVFEDLNIGFKRGRFKIEKQVYQKFELALAKKLNYLVSKEKGNYLNAFQFTPPVNNFQDIHKKQVGIIFYIPASFTSAICPVCGFRKRLYGFTFKNINQVKKLLAEHEFNIFYDGKRFNFECLASKENEKKENKNGLYTEIFLNKKLFFNSDIERLFNKKSKDNKKWETISFIATEELKKLFEGKIDLKRNIMDQILAGDFRAEFYQQLIFIINRILNLRNCHAVEHRDFIACPSCGFHSEKNYEKLKSRYAGKEKFEFNGDANGAYNIARKGILLIKKIRQFAKSNNIEKLMPYDHLQIDMQEWDKFCAKNKS

SEQ ID NO: 124 - protein, 1226 aa, synthetic construct
MKNFFQLFTNQYELSKTLRFELRPIENTAELLKKNKIFEKDKKVYEYYQKTKKYFDKLHCEFIDEALVNTSLPNKDYSIYDKLFFEYKKDKENKKIYKALNEHAKKLRTNIVKDFEKTAEKWRDEYILSIKDEKSQKKLKQLEGLDLLFKTEVFDFLKKKYPEAQIEGNSIFDSFNKFFTYFDNFHRSRKNFYKDDGTASAIPTRIIDDNLPKFLENKKIFEEEYKVLNLSREEKDLFRLDFYNKCFTQTGIENYNEIVSKINSKVNKYRQKNKKSKIHFMKELFKQILSKRSKQATEQDTSISIENDSQVFEVLQDFIMETKNCNRMAKNILETFIQHQEEFDLQKVFLKGSNVTRISDMWFTSWFAFGDLLPKNSTGKKLANFISFQDIKNALEKTEIQDVFKKKYAKYNKESFYDQFLQIFDYEFHKALNDCEKNIVEAEKMMKVDKVYSNKKGILRNGKNGEKQKEIIKDFADSALSIYQMMKYFALEKGKKPVTDMDEDPRFYNDYKDWCEKSRTLLYYNEFRNYLTKKPYAEDKIKLNFEKSTLLRGWSANREGNLQYYSSILRKDNKYYLGLMSSASTFNINKKEAFETRDGFYELMNYRQLKSTTIYGSLYKGEYKKEYRKDKETLTNQELIERTKKILEKQKFSYPQLEVILDKKYKAAEDLAKDLGNIVLYNIGFVKISKRYVNNLTNENFYLFEISNKDFLNKKKKGNVNLHTKYFQLLFDKKNLESKKGAILKLSGGGEVFYRKATKNLPKKKDKKGKEIIDRKRYAEDKILFHLPIVLNIGFKDKQINSKVNQVLATQRKARIIGIDRGEKHLLYISVIDENANILKIKSLNTIDVPNKKEPDDYHKLLDEKEKERDDERKSWHTIENIKELKHGYISQVVNEIDKIIFECLDERILPIIVFEDLNIGFKRGRFKIEKQVYQKFELALAKKLNYLVSKEKGNYLNAFQFTPPVNNFQDIHKKQVGIIFYIPASFTSAICPVCGFRKRLYGFTFKNINQVKKLLAEHEFNIFYDGKRFNFECLASKENEKKENKNGLYTEIFLNKKLFFNSDIERLFNKKSKDNKKWETISFIATEELKKLFEGKIDLKRNIMDQILAGDFRAEFYQQLIFIINRILNLRNCHAVEHRDFIACPSCGFHSEKNYEKLKSRYAGKEKFEFNGDANGAYNIARKGILLIKKIRQFAKSNNIEKLMPYDHLQIDMQEWDKFCAKNKS

SEQ ID NO: 125 - protein, 1226 aa, synthetic construct
MKNFFQLFTNQYELSKTLRFELRPIENTAELLKKNKIFEKDKKVYEYYQKTKKYFDKLHCEFIDEALVNTSLPNKDYSIYDKLFFEYKKDKENKKIYKALNEHAKKLRTNIVKDFEKTAEKWRDEYILSIKDEKSQKKLKQLEGLDLLFKTEVFDFLKKKYPEAQIEGNSIFDSFNKFFTYFDNFHRSRKNFYKDDGTASAIPTRIIDDNLPKFLENKKIFEEEYKVLNLSREEKDLFRLDFYNKCFTQTGIENYNEIVSKINSKVAKYRQKNKKSKIHFMKELFKQILSKRSKQATEQDTSISIENDSQVFEVLQDFIMETKNCNRMAKNILETFIQHQEEFDLQKVFLKGSNVTRISDMWFTSWFAFGDLLPKNSTGKKLANFISFQDIKNALEKTEIQDVFKKKYAKYNKESFYDQFLQIFDYEFHKALNDCEKNIVEAEKMMKVDKVYSNKKGILRNGKNGEKQKEIIKDFADSALSIYQMMKYFALEKGKKPVTDMDEDPRFYNDYKDWCEKSRTLLYYNEFRNYLTKKPYAEDKIKLNFEKSTLLMGWSANREGNLQYYSSILRKDNKYYLGLMSSASTFNINKKEAFETRDGFYELMNYRQLKSTTIYGSLYKGEYKKEYRKDKETLTNQELIERTKKILEKQKFSYPQLEVILDKKYKAAEDLAKDLGNIVLYNIGFVKISKRYVNNLTNENFYLFEISNKDFLNKKKKGNVNLHTKYFQLLFDKKNLESKKGAILKLSGGGEVFYRKATKNLPKKKDKKGKEIIDRKRYAEDKILFHLPIVLNIGFKDKQINSKVNQVLATQRKARIIGIDRGEKHLLYISVIDENANILKIKSLNTIDVPNKKEPDDYHKLLDEKEKERDDERKSWHTIENIKELKHGYISQVVNEIDKIIFECLDERILPIIVFEDLNIGFKRGRFKIEKQVYQKFELALAKKLNYLVSKEKGNYLNAFQFTPPVNNFQDIHKKQVGIIFYIPASFTSAICPVCGFRKRLYGFTFKNINQVKKLLAEHEFNIFYDGKRFNFECLASKENEKKENKNGLYTEIFLNKKLFFNSDIERLFNKKSKDNKKWETISFIATEELKKLFEGKIDLKRNIMDQILAGDFRAEFYQQLIFIINRILNLRNCHAVEHRDFIACPSCGFHSEKNYEKLKSRYAGKEKFEFNGDANGAYNIARKGILLIKKIRQFAKSNNIEKLMPYDHLQIDMQEWDKFCAKNKS

SEQ ID NO: 126 - protein, 1226 aa, synthetic construct
MKNFFQLFTNQYELSKTLRFELRPIENTAELLKKNKIFEKDKKVYEYYQKTKKYFDKLHCEFIDEALVNTSLPNKDYSIYDKLFFEYKKDKENKKIYKALNEHAKKLRTNIVKDFEKTAEKWRDEYILSIKDEKSQKKLKQLEGLDLLFKTEVFDFLKKKYPEAQIEGNSIFDSFNKFFTYFDNFHQSRKNFYKDDGTASAIPTRIIDDNLPKFLENKKIFEEEYKVLNLSREEKDLFRLDFYNKCFTQTGIENYNEIVSKINSKVNKYRQKNKKSKIHFMKELFKQILSKRSKQATEQDTSISIENDSQVFEVLQDFIMETKNCNRMAKNILETFIQHQEEFDLQKVFLKGSNVTRISDMWFTSWFAFGDLLPKNSTGKKLANFISFQDIKNALEKTEIQDVFKKKYAKYNKESFYDQFLQIFDYEFHKALNDCEKNIVEAEKMMKVDKVYSNKKGILRNGKNGEKQKEIIKDFADSALSIYQMMKYFALEKGKKPVTDMDEDPRFYNDYKDWCEKSRTLLYYNEFRNYLTKKPYAEDKIKLNFERSTLLMGWSANEEGNLQYYSSILRKDNKYYLGLMSSASTFNINKKEAFETRDGFYELMNYRQLKSTTIYGSLYKGEYKKEYRKDKETLTNQELIERTKKILEKQKFSYPQLEVILDKKYKAAEDLAKDLGNIVLYNIGFVKISKRYVNNLTNENFYLFEISNKDFLNKKKKGNVNLHTKYFQLLFDKKNLESKKGAILKLSGGGEVFYRKATKNLPKKKDKKGKEIIDRKRYAEDKILFHLPIVLNIGFKDKQINSKVNQVLATQRKARIIGIDRGEKHLLYISVIDENANILKIKSLNTIDVPNKKEPDDYHKLLDEKEKERDDERKSWHTIENIKELKHGYISQVVNEIDKIIFECLDERILPIIVFEDLNIGFKRGRFKIEKQVYQKFELALAKKLNYLVSKEKGNYLNAFQFTPPVNNFQDIHKKQVGIIFYIPASFTSAICPVCGFRKRLYGFTFKNINQVKKLLAEHEFNIFYDGKRFNFECLASKENEKKENKNGLYTEIFLNKKLFFNSDIERLFNKKSKDNKKWETISFIATEELKKLFEGKIDLKRNIMDQILAGDFRAEFYQQLIFIINRILNLRNCHAVEHRDFIACPSCGFHSEKNYEKLKSRYAGKEKFEFNGDANGAYNIARKGILLIKKIRQFAKSNNIEKLMPYDHLQIDMQEWDKFCAKNKS

SEQ ID NO: 127 - protein, 1226 aa, synthetic construct
MFNLYSNLRKLYPVQKTLRFELIPQGKTKKNMEKEGIIREDEHRAKIYNKVKKYCDEYHKIFIEDCLKDIKLDNLNKYYELYSIDKKDEDQKEEFTKTQEKLRKQISESFKNNNKYKGLFQKDIIDTYLINMYKEDKQKLQDINEFNKFTTYFTGYNRNRENMYLEEEKSTAIAYRLINENLPTFVDNLKIYNKLLKFIPEIIEKIHIELAEYIQVNSIDEIFDINYYNDVLTQKGIECYNIIISGKTKDEGTKIKGINEYINEYNQTHTNKIPKLRELYKQILSDIDTASFKFDVIEDDIQLLDSINLYYQQIFPIISKVNELFSKINNYNLDLIYINNDGTLSNVSNEIYSDWSYIRNHISEKYDTEYTGRLKVGTEQYSKQKQEYLRKQRNYSLSFLINCMKDDKIIEYLSNNMQQTGIVEKIKMDFDNFKSIQIQEDYKCLIKSENSIAKIKKLLDDTKLLQVFMKTLIPKDKTIEKDNEFYSELISYYDVFRDIIPLYNKTRNYLTQKPYSNEKIKINFACPTLLNGWDLNKEEANLGVILLKDEKYYLGIMNPYCKKIFNVDNRDSDTNNNYKKMEYKLLPGPNKMLPKVFFSKSRINEFAPSDELLEKYNKGYHKKGKDFDINFCHELIDFYKKSLNKHEDWKNFNFKFKNTVEYNDISEFYREVEEQGYKIEYKEYSEKYINELVERGELYLFQIYNKDFSEYSKGKQNLHTMYWKAVFDPENIKDTIYKLNGNAEIFYRKKSIDEKITHPANIPVANKNENTIKAGKITSLFKYNLIKDKRYTVDKFQFHVPITMNFKSEKILNINQIVNKYLKYNDDIHVIGIDRGERNLLYVCVIDKDEKIVYQKSLNEIYNEYQGIQYSTNYHKLLNEKENEREQAREDWKNIENIKELKEGYMSQIIHILCELMRKYNAIIVIEDLNKGFKNSRIKVEKQIYQKFEQMFINKLNYVVFKDVPKLEEGGILNAYQLTNKFESFTKLGKQSGILYYIPAWCTSKIDPTTGFINRFYIKYENLEKSREFVNRMDNIRYNLDEELFEFDIDYSKFSDRLNDTKNKWTLCSYGERVYTHKNNKEIWVDDKIQLTDEFIKLFEKYKIDLNNIKNEIIEKADIEFFKGGENSLGFLQLFKLMVQMRNSITGQEEDNLISPVRNNEGIFFDTKAKIKGLPEDADANGAYNIARKGLMLIEQMKRTDDDKLNKIKYDITEKEWLNYIQNRGV

SEQ ID NO: 128 - protein, 1226 aa, synthetic construct
MFNLYSNLRKLYPVQKTLRFELIPQGKTKKNMEKEGIIREDEHRAKIYNKVKKYCDEYHKIFIEDCLKDIKLDNLNKYYELYSIDKKDEDQKEEFTKTQEKLRKQISESFKNNNKYKGLFQKDIIDTYLINMYKEDKQKLQDINEFNKFTTYFTGYNKNRENMYLEEEKSTAIAYRLINENLPTFVDNLKIYNKLLKFIPEIIEKIHIELAEYIQVNSIDEIFDINYYNDVLTQKGIECYNIIISGKTKDEGTKIKGINEYINEYNQTHTNKIPKLRELYKQILSDIDTASFKFDVIEDDIQLLDSINLYYQQIFPIISKVNELFSKINNYNLDLIYINNDGTLSNVSNEIYSDWSYIRNHISEKYDTEYTGRLKVGTEQYSKQKQEYLRKQRNYSLSFLINCMKDDKIIEYLSNNMQQTGIVEKIKMDFDNFKSIQIQEDYKCLIKSENSIAKIKKLLDDTKLLQVFMKTLIPKDKTIEKDNEFYSELISYYDVFRDIIPLYNKTRNYLTQKPYSNEKIKINFACPTLLRGWDLNKEEANLGVILLKDEKYYLGIMNPYCKKIFNVDNRDSDTNNNYKKMEYKLLPGPNKMLPKVFFSKSRINEFAPSDELLEKYNKGYHKKGKDFDINFCHELIDFYKKSLNKHEDWKNFNFKFKNTVEYNDISEFYREVEEQGYKIEYKEYSEKYINELVERGELYLFQIYNKDFSEYSKGKQNLHTMYWKAVFDPENIKDTIYKLNGNAEIFYRKKSIDEKITHPANIPVANKNENTIKAGKITSLFKYNLIKDKRYTVDKFQFHVPITMNFKSEKILNINQIVNKYLKYNDDIHVIGIDRGERNLLYVCVIDKDEKIVYQKSLNEIYNEYQGIQYSTNYHKLLNEKENEREQAREDWKNIENIKELKEGYMSQIIHILCELMRKYNAIIVIEDLNKGFKNSRIKVEKQIYQKFEQMFINKLNYVVFKDVPKLEEGGILNAYQLTNKFESFTKLGKQSGILYYIPAWCTSKIDPTTGFINRFYIKYENLEKSREFVNRMDNIRYNLDEELFEFDIDYSKFSDRLNDTKNKWTLCSYGERVYTHKNNKEIWVDDKIQLTDEFIKLFEKYKIDLNNIKNEIIEKADIEFFKGGENSLGFLQLFKLMVQMRNSITGQEEDNLISPVRNNEGIFFDTKAKIKGLPEDADANGAYNIARKGLMLIEQMKRTDDDKLNKIKYDITEKEWLNYIQNRGV

SEQ ID NO: 129 - protein, 1226 aa, synthetic construct
MFNLYSNLRKLYPVQKTLRFELIPQGKTKKNMEKEGIIREDEHRAKIYNKVKKYCDEYHKIFIEDCLKDIKLDNLNKYYELYSIDKKDEDQKEEFTKTQEKLRKQISESFKNNNKYKGLFQKDIIDTYLINMYKEDKQKLQDINEFNKFTTYFTGYNKNRENMYLEEEKSTAIAYRLINENLPTFVDNLKIYNKLLKFIPEIIEKIHIELAEYIQVNSIDEIFDINYYNDVLTQKGIECYNIIISGKTKDEGTKIKGINEYINEYNQTHTNKIPKLRELYKQILSDIDTASFKFDVIEDDIQLLDSINLYYQQIFPIISKVNELFSKINNYNLDLIYINNDGTLSNVSNEIYSDWSYIRNHISEKYDTEYTGRLKVGTEQYSKQKQEYLRKQRNYSLSFLINCMKDDKIIEYLSNNMQQTGIVEKIKMDFDNFKSIQIQEDYKCLIKSENSIAKIKKLLDDTKLLQVFMKTLIPKDKTIEKDNEFYSELISYYDVFRDIIPLYNKTRNYLTQKPYSNEKIKINFACPTLLNGWDLNREEANLGVILLKDEKYYLGIMNPYCKKIFNVDNRDSDTNNNYKKMEYKLLPGPNKMLPKVFFSKSRINEFAPSDELLEKYNKGYHKKGKDFDINFCHELIDFYKKSLNKHEDWKNFNFKFKNTVEYNDISEFYREVEEQGYKIEYKEYSEKYINELVERGELYLFQIYNKDFSEYSKGKQNLHTMYWKAVFDPENIKDTIYKLNGNAEIFYRKKSIDEKITHPANIPVANKNENTIKAGKITSLFKYNLIKDKRYTVDKFQFHVPITMNFKSEKILNINQIVNKYLKYNDDIHVIGIDRGERNLLYVCVIDKDEKIVYQKSLNEIYNEYQGIQYSTNYHKLLNEKENEREQAREDWKNIENIKELKEGYMSQIIHILCELMRKYNAIIVIEDLNKGFKNSRIKVEKQIYQKFEQMFINKLNYVVFKDVPKLEEGGILNAYQLTNKFESFTKLGKQSGILYYIPAWCTSKIDPTTGFINRFYIKYENLEKSREFVNRMDNIRYNLDEELFEFDIDYSKFSDRLNDTKNKWTLCSYGERVYTHKNNKEIWVDDKIQLTDEFIKLFEKYKIDLNNIKNEIIEKADIEFFKGGENSLGFLQLFKLMVQMRNSITGQEEDNLISPVRNNEGIFFDTKAKIKGLPEDADANGAYNIARKGLMLIEQMKRTDDDKLNKIKYDITEKEWLNYIQNRGV

SEQ ID NO: 130 - protein, 1226 aa, synthetic construct
MFNLYSNLRKLYPVQKTLRFELIPQGKTKKNMEKEGIIREDEHRAKIYNKVKKYCDEYHKIFIEDCLKDIKLDNLNKYYELYSIDKKDEDQKEEFTKTQEKLRKQISESFKNNNKYKGLFQKDIIDTYLINMYKEDKQKLQDINEFNKFTTYFTGYNRNRENMYLEEEKSTAIAYRLINENLPTFVDNLKIYNKLLKFIPEIIEKIHIELAEYIQVNSIDEIFDINYYNDVLTQKGIECYNIIISGKTKDEGTKIKGINEYINEYNQTHTNKIPKLRELYKQILSDIDTASFKFDVIEDDIQLLDSINLYYQQIFPIISKVNELFSKINNYNLDLIYINNDGTLSNVSNEIYSDWSYIRNHISEKYDTEYTGRLKVGTEQYSKQKQEYLRKQRNYSLSFLINCMKDDKIIEYLSNNMQQTGIVEKIKMDFDNFKSIQIQEDYKCLIKSENSIAKIKKLLDDTKLLQVFMKTLIPKDKTIEKDNEFYSELISYYDVFRDIIPLYNKTRNYLTQKPYSNEKIKINFACPTLLRGWDLNKEEANLGVILLKDEKYYLGIMNPYCKKIFNVDNRDSDTNNNYKKMEYKLLPGPNKMLPKVFFSKSRINEFAPSDELLEKYNKGYHKKGKDFDINFCHELIDFYKKSLNKHEDWKNFNFKFKNTVEYNDISEFYREVEEQGYKIEYKEYSEKYINELVERGELYLFQIYNKDFSEYSKGKQNLHTMYWKAVFDPENIKDTIYKLNGNAEIFYRKKSIDEKITHPANIPVANKNENTIKAGKITSLFKYNLIKDKRYTVDKFQFHVPITMNFKSEKILNINQIVNKYLKYNDDIHVIGIDRGERNLLYVCVIDKDEKIVYQKSLNEIYNEYQGIQYSTNYHKLLNEKENEREQAREDWKNIENIKELKEGYMSQIIHILCELMRKYNAIIVIEDLNKGFKNSRIKVEKQIYQKFEQMFINKLNYVVFKDVPKLEEGGILNAYQLTNKFESFTKLGKQSGILYYIPAWCTSKIDPTTGFINRFYIKYENLEKSREFVNRMDNIRYNLDEELFEFDIDYSKFSDRLNDTKNKWTLCSYGERVYTHKNNKEIWVDDKIQLTDEFIKLFEKYKIDLNNIKNEIIEKADIEFFKGGENSLGFLQLFKLMVQMRNSITGQEEDNLISPVRNNEGIFFDTKAKIKGLPEDADANGAYNIARKGLMLIEQMKRTDDDKLNKIKYDITEKEWLNYIQNRGV

SEQ ID NO: 131 - protein, 1226 aa, synthetic construct
MFNLYSNLRKLYPVQKTLRFELIPQGKTKKNMEKEGIIREDEHRAKIYNKVKKYCDEYHKIFIEDCLKDIKLDNLNKYYELYSIDKKDEDQKEEFTKTQEKLRKQISESFKNNNKYKGLFQKDIIDTYLINMYKEDKQKLQDINEFNKFTTYFTGYNRNRENMYLEEEKSTAIAYRLINENLPTFVDNLKIYNKLLKFIPEIIEKIHIELAEYIQVNSIDEIFDINYYNDVLTQKGIECYNIIISGKTKDEGTKIKGINEYINEYNQTHTNKIPKLRELYKQILSDIDTASFKFDVIEDDIQLLDSINLYYQQIFPIISKVNELFSKINNYNLDLIYINNDGTLSNVSNEIYSDWSYIRNHISEKYDTEYTGRLKVGTEQYSKQKQEYLRKQRNYSLSFLINCMKDDKIIEYLSNNMQQTGIVEKIKMDFDNFKSIQIQEDYKCLIKSENSIAKIKKLLDDTKLLQVFMKTLIPKDKTIEKDNEFYSELISYYDVFRDIIPLYNKTRNYLTQKPYSNEKIKINFACPTLLNGWDLNREEANLGVILLKDEKYYLGIMNPYCKKIFNVDNRDSDTNNNYKKMEYKLLPGPNKMLPKVFFSKSRINEFAPSDELLEKYNKGYHKKGKDFDINFCHELIDFYKKSLNKHEDWKNFNFKFKNTVEYNDISEFYREVEEQGYKIEYKEYSEKYINELVERGELYLFQIYNKDFSEYSKGKQNLHTMYWKAVFDPENIKDTIYKLNGNAEIFYRKKSIDEKITHPANIPVANKNENTIKAGKITSLFKYNLIKDKRYTVDKFQFHVPITMNFKSEKILNINQIVNKYLKYNDDIHVIGIDRGERNLLYVCVIDKDEKIVYQKSLNEIYNEYQGIQYSTNYHKLLNEKENEREQAREDWKNIENIKELKEGYMSQIIHILCELMRKYNAIIVIEDLNKGFKNSRIKVEKQIYQKFEQMFINKLNYVVFKDVPKLEEGGILNAYQLTNKFESFTKLGKQSGILYYIPAWCTSKIDPTTGFINRFYIKYENLEKSREFVNRMDNIRYNLDEELFEFDIDYSKFSDRLNDTKNKWTLCSYGERVYTHKNNKEIWVDDKIQLTDEFIKLFEKYKIDLNNIKNEIIEKADIEFFKGGENSLGFLQLFKLMVQMRNSITGQEEDNLISPVRNNEGIFFDTKAKIKGLPEDADANGAYNIARKGLMLIEQMKRTDDDKLNKIKYDITEKEWLNYIQNRGV

SEQ ID NO: 132 - protein, 1226 aa, synthetic construct
MFNLYSNLRKLYPVQKTLRFELIPQGKTKKNMEKEGIIREDEHRAKIYNKVKKYCDEYHKIFIEDCLKDIKLDNLNKYYELYSIDKKDEDQKEEFTKTQEKLRKQISESFKNNNKYKGLFQKDIIDTYLINMYKEDKQKLQDINEFNKFTTYFTGYNKNRENMYLEEEKSTAIAYRLINENLPTFVDNLKIYNKLLKFIPEIIEKIHIELAEYIQVNSIDEIFDINYYNDVLTQKGIECYNIIISGKTKDEGTKIKGINEYINEYNQTHTNKIPKLRELYKQILSDIDTASFKFDVIEDDIQLLDSINLYYQQIFPIISKVNELFSKINNYNLDLIYINNDGTLSNVSNEIYSDWSYIRNHISEKYDTEYTGRLKVGTEQYSKQKQEYLRKQRNYSLSFLINCMKDDKIIEYLSNNMQQTGIVEKIKMDFDNFKSIQIQEDYKCLIKSENSIAKIKKLLDDTKLLQVFMKTLIPKDKTIEKDNEFYSELISYYDVFRDIIPLYNKTRNYLTQKPYSNEKIKINFACPTLLRGWDLNREEANLGVILLKDEKYYLGIMNPYCKKIFNVDNRDSDTNNNYKKMEYKLLPGPNKMLPKVFFSKSRINEFAPSDELLEKYNKGYHKKGKDFDINFCHELIDFYKKSLNKHEDWKNFNFKFKNTVEYNDISEFYREVEEQGYKIEYKEYSEKYINELVERGELYLFQIYNKDFSEYSKGKQNLHTMYWKAVFDPENIKDTIYKLNGNAEIFYRKKSIDEKITHPANIPVANKNENTIKAGKITSLFKYNLIKDKRYTVDKFQFHVPITMNFKSEKILNINQIVNKYLKYNDDIHVIGIDRGERNLLYVCVIDKDEKIVYQKSLNEIYNEYQGIQYSTNYHKLLNEKENEREQAREDWKNIENIKELKEGYMSQIIHILCELMRKYNAIIVIEDLNKGFKNSRIKVEKQIYQKFEQMFINKLNYVVFKDVPKLEEGGILNAYQLTNKFESFTKLGKQSGILYYIPAWCTSKIDPTTGFINRFYIKYENLEKSREFVNRMDNIRYNLDEELFEFDIDYSKFSDRLNDTKNKWTLCSYGERVYTHKNNKEIWVDDKIQLTDEFIKLFEKYKIDLNNIKNEIIEKADIEFFKGGENSLGFLQLFKLMVQMRNSITGQEEDNLISPVRNNEGIFFDTKAKIKGLPEDADANGAYNIARKGLMLIEQMKRTDDDKLNKIKYDITEKEWLNYIQNRGV

SEQ ID NO: 133 - protein, 1226 aa, synthetic construct
MFNLYSNLRKLYPVQKTLRFELIPQGKTKKNMEKEGIIREDEHRAKIYNKVKKYCDEYHKIFIEDCLKDIKLDNLNKYYELYSIDKKDEDQKEEFTKTQEKLRKQISESFKNNNKYKGLFQKDIIDTYLINMYKEDKQKLQDINEFNKFTTYFTGYNRNRENMYLEEEKSTAIAYRLINENLPTFVDNLKIYNKLLKFIPEIIEKIHIELAEYIQVNSIDEIFDINYYNDVLTQKGIECYNIIISGKTKDEGTKIKGINEYINEYNQTHTNKIPKLRELYKQILSDIDTASFKFDVIEDDIQLLDSINLYYQQIFPIISKVNELFSKINNYNLDLIYINNDGTLSNVSNEIYSDWSYIRNHISEKYDTEYTGRLKVGTEQYSKQKQEYLRKQRNYSLSFLINCMKDDKIIEYLSNNMQQTGIVEKIKMDFDNFKSIQIQEDYKCLIKSENSIAKIKKLLDDTKLLQVFMKTLIPKDKTIEKDNEFYSELISYYDVFRDIIPLYNKTRNYLTQKPYSNEKIKINFACPTLLRGWDLNREEANLGVILLKDEKYYLGIMNPYCKKIFNVDNRDSDTNNNYKKMEYKLLPGPNKMLPKVFFSKSRINEFAPSDELLEKYNKGYHKKGKDFDINFCHELIDFYKKSLNKHEDWKNFNFKFKNTVEYNDISEFYREVEEQGYKIEYKEYSEKYINELVERGELYLFQIYNKDFSEYSKGKQNLHTMYWKAVFDPENIKDTIYKLNGNAEIFYRKKSIDEKITHPANIPVANKNENTIKAGKITSLFKYNLIKDKRYTVDKFQFHVPITMNFKSEKILNINQIVNKYLKYNDDIHVIGIDRGERNLLYVCVIDKDEKIVYQKSLNEIYNEYQGIQYSTNYHKLLNEKENEREQAREDWKNIENIKELKEGYMSQIIHILCELMRKYNAIIVIEDLNKGFKNSRIKVEKQIYQKFEQMFINKLNYVVFKDVPKLEEGGILNAYQLTNKFESFTKLGKQSGILYYIPAWCTSKIDPTTGFINRFYIKYENLEKSREFVNRMDNIRYNLDEELFEFDIDYSKFSDRLNDTKNKWTLCSYGERVYTHKNNKEIWVDDKIQLTDEFIKLFEKYKIDLNNIKNEIIEKADIEFFKGGENSLGFLQLFKLMVQMRNSITGQEEDNLISPVRNNEGIFFDTKAKIKGLPEDADANGAYNIARKGLMLIEQMKRTDDDKLNKIKYDITEKEWLNYIQNRGV

SEQ ID NO: 134 - protein, 1226 aa, synthetic construct
MFNLYSNLRKLYPVQKTLRFELIPQGKTKKNMEKEGIIREDEHRAKIYNKVKKYCDEYHKIFIEDCLKDIKLDNLNKYYELYSIDKKDEDQKEEFTKTQEKLRKQISESFKNNNKYKGLFQKDIIDTYLINMYKEDKQKLQDINEFNKFTTYFTGYNRNRENMYLEEEKSTAIAYRLINENLPTFVDNLKIYNKLLKFIPEIIEKIHIELAEYIQVNSIDEIFDINYYNDVLTQKGIECYNIIISGKTKDEGTKIKGINEYIAEYNQTHTNKIPKLRELYKQILSDIDTASFKFDVIEDDIQLLDSINLYYQQIFPIISKVNELFSKINNYNLDLIYINNDGTLSNVSNEIYSDWSYIRNHISEKYDTEYTGRLKVGTEQYSKQKQEYLRKQRNYSLSFLINCMKDDKIIEYLSNNMQQTGIVEKIKMDFDNFKSIQIQEDYKCLIKSENSIAKIKKLLDDTKLLQVFMKTLIPKDKTIEKDNEFYSELISYYDVFRDIIPLYNKTRNYLTQKPYSNEKIKINFACPTLLNGWDLNREEANLGVILLKDEKYYLGIMNPYCKKIFNVDNRDSDTNNNYKKMEYKLLPGPNKMLPKVFFSKSRINEFAPSDELLEKYNKGYHKKGKDFDINFCHELIDFYKKSLNKHEDWKNFNFKFKNTVEYNDISEFYREVEEQGYKIEYKEYSEKYINELVERGELYLFQIYNKDFSEYSKGKQNLHTMYWKAVFDPENIKDTIYKLNGNAEIFYRKKSIDEKITHPANIPVANKNENTIKAGKITSLFKYNLIKDKRYTVDKFQFHVPITMNFKSEKILNINQIVNKYLKYNDDIHVIGIDRGERNLLYVCVIDKDEKIVYQKSLNEIYNEYQGIQYSTNYHKLLNEKENEREQAREDWKNIENIKELKEGYMSQIIHILCELMRKYNAIIVIEDLNKGFKNSRIKVEKQIYQKFEQMFINKLNYVVFKDVPKLEEGGILNAYQLTNKFESFTKLGKQSGILYYIPAWCTSKIDPTTGFINRFYIKYENLEKSREFVNRMDNIRYNLDEELFEFDIDYSKFSDRLNDTKNKWTLCSYGERVYTHKNNKEIWVDDKIQLTDEFIKLFEKYKIDLNNIKNEIIEKADIEFFKGGENSLGFLQLFKLMVQMRNSITGQEEDNLISPVRNNEGIFFDTKAKIKGLPEDADANGAYNIARKGLMLIEQMKRTDDDKLNKIKYDITEKEWLNYIQNRGV

SEQ ID NO: 135 - protein, 1226 aa, synthetic construct
MFNLYSNLRKLYPVQKTLRFELIPQGKTKKNMEKEGIIREDEHRAKIYNKVKKYCDEYHKIFIEDCLKDIKLDNLNKYYELYSIDKKDEDQKEEFTKTQEKLRKQISESFKNNNKYKGLFQKDIIDTYLINMYKEDKQKLQDINEFNKFTTYFTGYNKNRENMYLEEEKSTAIAYRLINENLPTFVDNLKIYNKLLKFIPEIIEKIHIELAEYIQVNSIDEIFDINYYNDVLTQKGIECYNIIISGKTKDEGTKIKGINEYINEYNQTHTNKIPKLRELYKQILSDIDTASFKFDVIEDDIQLLDSINLYYQQIFPIISKVNELFSKINNYNLDLIYINNDGTLSNVSNEIYSDWSYIRNHISEKYDTEYTGRLKVGTEQYSKQKQEYLRKQRNYSLSFLINCMKDDKIIEYLSNNMQQTGIVEKIKMDFDNFKSIQIQEDYKCLIKSENSIAKIKKLLDDTKLLQVFMKTLIPKDKTIEKDNEFYSELISYYDVFRDIIPLYNKTRNYLTQKPYSNEKIKINFARPTLLNGWDLNKEEANLGVILLKDEKYYLGIMNPYCKKIFNVDNRDSDTNNNYKKMEYKLLPGPNKMLPKVFFSKSRINEFAPSDELLEKYNKGYHKKGKDFDINFCHELIDFYKKSLNKHEDWKNFNFKFKNTVEYNDISEFYREVEEQGYKIEYKEYSEKYINELVERGELYLFQIYNKDFSEYSKGKQNLHTMYWKAVFDPENIKDTIYKLNGNAEIFYRKKSIDEKITHPANIPVANKNENTIKAGKITSLFKYNLIKDKRYTVDKFQFHVPITMNFKSEKILNINQIVNKYLKYNDDIHVIGIDRGERNLLYVCVIDKDEKIVYQKSLNEIYNEYQGIQYSTNYHKLLNEKENEREQAREDWKNIENIKELKEGYMSQIIHILCELMRKYNAIIVIEDLNKGFKNSRIKVEKQIYQKFEQMFINKLNYVVFKDVPKLEEGGILNAYQLTNKFESFTKLGKQSGILYYIPAWCTSKIDPTTGFINRFYIKYENLEKSREFVNRMDNIRYNLDEELFEFDIDYSKFSDRLNDTKNKWTLCSYGERVYTHKNNKEIWVDDKIQLTDEFIKLFEKYKIDLNNIKNEIIEKADIEFFKGGENSLGFLQLFKLMVQMRNSITGQEEDNLISPVRNNEGIFFDTKAKIKGLPEDADANGAYNIARKGLMLIEQMKRTDDDKLNKIKYDITEKEWLNYIQNRGV

SEQ ID NO: 136 - protein, 1226 aa, synthetic construct
MSNLYRNLHNFYSVQKTLRFELIPQGKTKENMEKEGILKADEHRAEIYSKVKKYCDEYHKLFIDKCLKNIRLNELNKYYELYSVVKKDEKQKEEFIKIQEKMRKQISESFKNDNEYKGLFQKDIINIYLITMYKDDKEKIKDISEFNKFTTYFSGYNRNRENMYSEEEKPTGIAYRLINENLPTFIENFKIYNKVIKFMPEIINKIHTDLMEYIQVEDIDEIFDINYYNEVLTQKGIECYNIIISGKSKSNGEKIKGLNEYINEFNQKHNEKIPKLQELYKQILSDTDTASFKFDTIESDEELLNNIESYYTKLLPVFNKINQLFAKFNKYNLDLIFINNDGTLNTISNEIYKDWSYIRNRIGERYDIEYTGKLKKDTEQYSKQKQEYMKKQKQYSLKFLNDSLRDNYLIEYISNYIEQSKIMEKMKTDFTEVQKIESRGDTKQLIKDENSIVKIKNLLDDIKFLQEFVKILVLKDRTIEKDAEFYSELMPYYNELKDIIPLYNKTRNYLTQKPYSTEKIKLNFECPTLLNGWDLNKEEANLGVILLKNEKYYLGIINPYCKKIFKIQEKDSNSENNYKKMEYKLLPGPNKMLPKVFFSKSKIDKFMPSDELLEKYNKGCHKKGKDFDINFCHELIDFYKTSLNKHEDWKKFDFKFKSTSEYNDISEFYKDVEEQGYKIEYSEYSEKYINELVDRGELYLFQIYNKDFSEYSKGRPNLHTMYWKAVFDIENIKNPVYKLNGEAEIFYRKKSLERKITHSANEPVANKNENTIKSGKPTSLFKYDLIKDKRYTVDKFQFHVPITMNFKSEKMFNINQVVNKYLKYNDDINVIGIDRGERNLLYVCVIDKNEKIVYQKSLNEIVNEYKSIKYSTNYHALLNKKEKEREIAREDWKNIENIKELKEGYMSQVIHILVELMRKYNAIIVIEDLNKGFKNSRIKVEKQVYQKFEKMFIDKLNYLVFKDEPKESEGGVLNAYQLTNKFETFNKIGKQSGVLYYIPAWCTSKIDPTTGFINRFYIKYENLDKSKEFINKIDDISYNSLENLFEFDIDYSKFTDRLNETRNKWTLYTNGERIYTYRNDKGEWIDKKIQLTNEFKKLFEKYSINLDNIKNEILEKANIEFFKGNNETLGFIQLFKLMVQMRNSLTGKEEDNLISPVKNSNGKFFNTNEQIEGLPKDADANGAYNIARKGLMLIEQMRNTEDDKLNKIKYNITEKEWLDYVQNRGM

SEQ ID NO: 137 - protein, 1226 aa, synthetic construct
MSNLYRNLHNFYSVQKTLRFELIPQGKTKENMEKEGILKADEHRAEIYSKVKKYCDEYHKLFIDKCLKNIRLNELNKYYELYSVVKKDEKQKEEFIKIQEKMRKQISESFKNDNEYKGLFQKDIINIYLITMYKDDKEKIKDISEFNKFTTYFSGYNKNRENMYSEEEKPTGIAYRLINENLPTFIENFKIYNKVIKFMPEIINKIHTDLMEYIQVEDIDEIFDINYYNEVLTQKGIECYNIIISGKSKSNGEKIKGLNEYINEFNQKHNEKIPKLQELYKQILSDTDTASFKFDTIESDEELLNNIESYYTKLLPVFNKINQLFAKFNKYNLDLIFINNDGTLNTISNEIYKDWSYIRNRIGERYDIEYTGKLKKDTEQYSKQKQEYMKKQKQYSLKFLNDSLRDNYLIEYISNYIEQSKIMEKMKTDFTEVQKIESRGDTKQLIKDENSIVKIKNLLDDIKFLQEFVKILVLKDRTIEKDAEFYSELMPYYNELKDIIPLYNKTRNYLTQKPYSTEKIKLNFECPTLLRGWDLNKEEANLGVILLKNEKYYLGIINPYCKKIFKIQEKDSNSENNYKKMEYKLLPGPNKMLPKVFFSKSKIDKFMPSDELLEKYNKGCHKKGKDFDINFCHELIDFYKTSLNKHEDWKKFDFKFKSTSEYNDISEFYKDVEEQGYKIEYSEYSEKYINELVDRGELYLFQIYNKDFSEYSKGRPNLHTMYWKAVFDIENIKNPVYKLNGEAEIFYRKKSLERKITHSANEPVANKNENTIKSGKPTSLFKYDLIKDKRYTVDKFQFHVPITMNFKSEKMFNINQVVNKYLKYNDDINVIGIDRGERNLLYVCVIDKNEKIVYQKSLNEIVNEYKSIKYSTNYHALLNKKEKEREIAREDWKNIENIKELKEGYMSQVIHILVELMRKYNAIIVIEDLNKGFKNSRIKVEKQVYQKFEKMFIDKLNYLVFKDEPKESEGGVLNAYQLTNKFETFNKIGKQSGVLYYIPAWCTSKIDPTTGFINRFYIKYENLDKSKEFINKIDDISYNSLENLFEFDIDYSKFTDRLNETRNKWTLYTNGERIYTYRNDKGEWIDKKIQLTNEFKKLFEKYSINLDNIKNEILEKANIEFFKGNNETLGFIQLFKLMVQMRNSLTGKEEDNLISPVKNSNGKFFNTNEQIEGLPKDADANGAYNIARKGLMLIEQMRNTEDDKLNKIKYNITEKEWLDYVQNRGM

SEQ ID NO: 138 - protein, 1226 aa, synthetic construct
MSNLYRNLHNFYSVQKTLRFELIPQGKTKENMEKEGILKADEHRAEIYSKVKKYCDEYHKLFIDKCLKNIRLNELNKYYELYSVVKKDEKQKEEFIKIQEKMRKQISESFKNDNEYKGLFQKDIINIYLITMYKDDKEKIKDISEFNKFTTYFSGYNKNRENMYSEEEKPTGIAYRLINENLPTFIENFKIYNKVIKFMPEIINKIHTDLMEYIQVEDIDEIFDINYYNEVLTQKGIECYNIIISGKSKSNGEKIKGLNEYINEFNQKHNEKIPKLQELYKQILSDTDTASFKFDTIESDEELLNNIESYYTKLLPVFNKINQLFAKFNKYNLDLIFINNDGTLNTISNEIYKDWSYIRNRIGERYDIEYTGKLKKDTEQYSKQKQEYMKKQKQYSLKFLNDSLRDNYLIEYISNYIEQSKIMEKMKTDFTEVQKIESRGDTKQLIKDENSIVKIKNLLDDIKFLQEFVKILVLKDRTIEKDAEFYSELMPYYNELKDIIPLYNKTRNYLTQKPYSTEKIKLNFECPTLLNGWDLNREEANLGVILLKNEKYYLGIINPYCKKIFKIQEKDSNSENNYKKMEYKLLPGPNKMLPKVFFSKSKIDKFMPSDELLEKYNKGCHKKGKDFDINFCHELIDFYKTSLNKHEDWKKFDFKFKSTSEYNDISEFYKDVEEQGYKIEYSEYSEKYINELVDRGELYLFQIYNKDFSEYSKGRPNLHTMYWKAVFDIENIKNPVYKLNGEAEIFYRKKSLERKITHSANEPVANKNENTIKSGKPTSLFKYDLIKDKRYTVDKFQFHVPITMNFKSEKMFNINQVVNKYLKYNDDINVIGIDRGERNLLYVCVIDKNEKIVYQKSLNEIVNEYKSIKYSTNYHALLNKKEKEREIAREDWKNIENIKELKEGYMSQVIHILVELMRKYNAIIVIEDLNKGFKNSRIKVEKQVYQKFEKMFIDKLNYLVFKDEPKESEGGVLNAYQLTNKFETFNKIGKQSGVLYYIPAWCTSKIDPTTGFINRFYIKYENLDKSKEFINKIDDISYNSLENLFEFDIDYSKFTDRLNETRNKWTLYTNGERIYTYRNDKGEWIDKKIQLTNEFKKLFEKYSINLDNIKNEILEKANIEFFKGNNETLGFIQLFKLMVQMRNSLTGKEEDNLISPVKNSNGKFFNTNEQIEGLPKDADANGAYNIARKGLMLIEQMRNTEDDKLNKIKYNITEKEWLDYVQNRGM

SEQ ID NO: 139 - protein, 1226 aa, synthetic construct
MSNLYRNLHNFYSVQKTLRFELIPQGKTKENMEKEGILKADEHRAEIYSKVKKYCDEYHKLFIDKCLKNIRLNELNKYYELYSVVKKDEKQKEEFIKIQEKMRKQISESFKNDNEYKGLFQKDIINIYLITMYKDDKEKIKDISEFNKFTTYFSGYNRNRENMYSEEEKPTGIAYRLINENLPTFIENFKIYNKVIKFMPEIINKIHTDLMEYIQVEDIDEIFDINYYNEVLTQKGIECYNIIISGKSKSNGEKIKGLNEYINEFNQKHNEKIPKLQELYKQILSDTDTASFKFDTIESDEELLNNIESYYTKLLPVFNKINQLFAKFNKYNLDLIFINNDGTLNTISNEIYKDWSYIRNRIGERYDIEYTGKLKKDTEQYSKQKQEYMKKQKQYSLKFLNDSLRDNYLIEYISNYIEQSKIMEKMKTDFTEVQKIESRGDTKQLIKDENSIVKIKNLLDDIKFLQEFVKILVLKDRTIEKDAEFYSELMPYYNELKDIIPLYNKTRNYLTQKPYSTEKIKLNFECPTLLRGWDLNKEEANLGVILLKNEKYYLGIINPYCKKIFKIQEKDSNSENNYKKMEYKLLPGPNKMLPKVFFSKSKIDKFMPSDELLEKYNKGCHKKGKDFDINFCHELIDFYKTSLNKHEDWKKFDFKFKSTSEYNDISEFYKDVEEQGYKIEYSEYSEKYINELVDRGELYLFQIYNKDFSEYSKGRPNLHTMYWKAVFDIENIKNPVYKLNGEAEIFYRKKSLERKITHSANEPVANKNENTIKSGKPTSLFKYDLIKDKRYTVDKFQFHVPITMNFKSEKMFNINQVVNKYLKYNDDINVIGIDRGERNLLYVCVIDKNEKIVYQKSLNEIVNEYKSIKYSTNYHALLNKKEKEREIAREDWKNIENIKELKEGYMSQVIHILVELMRKYNAIIVIEDLNKGFKNSRIKVEKQVYQKFEKMFIDKLNYLVFKDEPKESEGGVLNAYQLTNKFETFNKIGKQSGVLYYIPAWCTSKIDPTTGFINRFYIKYENLDKSKEFINKIDDISYNSLENLFEFDIDYSKFTDRLNETRNKWTLYTNGERIYTYRNDKGEWIDKKIQLTNEFKKLFEKYSINLDNIKNEILEKANIEFFKGNNETLGFIQLFKLMVQMRNSLTGKEEDNLISPVKNSNGKFFNTNEQIEGLPKDADANGAYNIARKGLMLIEQMRNTEDDKLNKIKYNITEKEWLDYVQNRGM

SEQ ID NO: 140 - protein, 1226 aa, synthetic construct
MSNLYRNLHNFYSVQKTLRFELIPQGKTKENMEKEGILKADEHRAEIYSKVKKYCDEYHKLFIDKCLKNIRLNELNKYYELYSVVKKDEKQKEEFIKIQEKMRKQISESFKNDNEYKGLFQKDIINIYLITMYKDDKEKIKDISEFNKFTTYFSGYNRNRENMYSEEEKPTGIAYRLINENLPTFIENFKIYNKVIKFMPEIINKIHTDLMEYIQVEDIDEIFDINYYNEVLTQKGIECYNIIISGKSKSNGEKIKGLNEYINEFNQKHNEKIPKLQELYKQILSDTDTASFKFDTIESDEELLNNIESYYTKLLPVFNKINQLFAKFNKYNLDLIFINNDGTLNTISNEIYKDWSYIRNRIGERYDIEYTGKLKKDTEQYSKQKQEYMKKQKQYSLKFLNDSLRDNYLIEYISNYIEQSKIMEKMKTDFTEVQKIESRGDTKQLIKDENSIVKIKNLLDDIKFLQEFVKILVLKDRTIEKDAEFYSELMPYYNELKDIIPLYNKTRNYLTQKPYSTEKIKLNFECPTLLNGWDLNREEANLGVILLKNEKYYLGIINPYCKKIFKIQEKDSNSENNYKKMEYKLLPGPNKMLPKVFFSKSKIDKFMPSDELLEKYNKGCHKKGKDFDINFCHELIDFYKTSLNKHEDWKKFDFKFKSTSEYNDISEFYKDVEEQGYKIEYSEYSEKYINELVDRGELYLFQIYNKDFSEYSKGRPNLHTMYWKAVFDIENIKNPVYKLNGEAEIFYRKKSLERKITHSANEPVANKNENTIKSGKPTSLFKYDLIKDKRYTVDKFQFHVPITMNFKSEKMFNINQVVNKYLKYNDDINVIGIDRGERNLLYVCVIDKNEKIVYQKSLNEIVNEYKSIKYSTNYHALLNKKEKEREIAREDWKNIENIKELKEGYMSQVIHILVELMRKYNAIIVIEDLNKGFKNSRIKVEKQVYQKFEKMFIDKLNYLVFKDEPKESEGGVLNAYQLTNKFETFNKIGKQSGVLYYIPAWCTSKIDPTTGFINRFYIKYENLDKSKEFINKIDDISYNSLENLFEFDIDYSKFTDRLNETRNKWTLYTNGERIYTYRNDKGEWIDKKIQLTNEFKKLFEKYSINLDNIKNEILEKANIEFFKGNNETLGFIQLFKLMVQMRNSLTGKEEDNLISPVKNSNGKFFNTNEQIEGLPKDADANGAYNIARKGLMLIEQMRNTEDDKLNKIKYNITEKEWLDYVQNRGM

SEQ ID NO: 141 - protein, 1226 aa, synthetic construct
MSNLYRNLHNFYSVQKTLRFELIPQGKTKENMEKEGILKADEHRAEIYSKVKKYCDEYHKLFIDKCLKNIRLNELNKYYELYSVVKKDEKQKEEFIKIQEKMRKQISESFKNDNEYKGLFQKDIINIYLITMYKDDKEKIKDISEFNKFTTYFSGYNKNRENMYSEEEKPTGIAYRLINENLPTFIENFKIYNKVIKFMPEIINKIHTDLMEYIQVEDIDEIFDINYYNEVLTQKGIECYNIIISGKSKSNGEKIKGLNEYINEFNQKHNEKIPKLQELYKQILSDTDTASFKFDTIESDEELLNNIESYYTKLLPVFNKINQLFAKFNKYNLDLIFINNDGTLNTISNEIYKDWSYIRNRIGERYDIEYTGKLKKDTEQYSKQKQEYMKKQKQYSLKFLNDSLRDNYLIEYISNYIEQSKIMEKMKTDFTEVQKIESRGDTKQLIKDENSIVKIKNLLDDIKFLQEFVKILVLKDRTIEKDAEFYSELMPYYNELKDIIPLYNKTRNYLTQKPYSTEKIKLNFECPTLLRGWDLNREEANLGVILLKNEKYYLGIINPYCKKIFKIQEKDSNSENNYKKMEYKLLPGPNKMLPKVFFSKSKIDKFMPSDELLEKYNKGCHKKGKDFDINFCHELIDFYKTSLNKHEDWKKFDFKFKSTSEYNDISEFYKDVEEQGYKIEYSEYSEKYINELVDRGELYLFQIYNKDFSEYSKGRPNLHTMYWKAVFDIENIKNPVYKLNGEAEIFYRKKSLERKITHSANEPVANKNENTIKSGKPTSLFKYDLIKDKRYTVDKFQFHVPITMNFKSEKMFNINQVVNKYLKYNDDINVIGIDRGERNLLYVCVIDKNEKIVYQKSLNEIVNEYKSIKYSTNYHALLNKKEKEREIAREDWKNIENIKELKEGYMSQVIHILVELMRKYNAIIVIEDLNKGFKNSRIKVEKQVYQKFEKMFIDKLNYLVFKDEPKESEGGVLNAYQLTNKFETFNKIGKQSGVLYYIPAWCTSKIDPTTGFINRFYIKYENLDKSKEFINKIDDISYNSLENLFEFDIDYSKFTDRLNETRNKWTLYTNGERIYTYRNDKGEWIDKKIQLTNEFKKLFEKYSINLDNIKNEILEKANIEFFKGNNETLGFIQLFKLMVQMRNSLTGKEEDNLISPVKNSNGKFFNTNEQIEGLPKDADANGAYNIARKGLMLIEQMRNTEDDKLNKIKYNITEKEWLDYVQNRGM

SEQ ID NO: 142 - protein, 1226 aa, synthetic construct
MSNLYRNLHNFYSVQKTLRFELIPQGKTKENMEKEGILKADEHRAEIYSKVKKYCDEYHKLFIDKCLKNIRLNELNKYYELYSVVKKDEKQKEEFIKIQEKMRKQISESFKNDNEYKGLFQKDIINIYLITMYKDDKEKIKDISEFNKFTTYFSGYNRNRENMYSEEEKPTGIAYRLINENLPTFIENFKIYNKVIKFMPEIINKIHTDLMEYIQVEDIDEIFDINYYNEVLTQKGIECYNIIISGKSKSNGEKIKGLNEYINEFNQKHNEKIPKLQELYKQILSDTDTASFKFDTIESDEELLNNIESYYTKLLPVFNKINQLFAKFNKYNLDLIFINNDGTLNTISNEIYKDWSYIRNRIGERYDIEYTGKLKKDTEQYSKQKQEYMKKQKQYSLKFLNDSLRDNYLIEYISNYIEQSKIMEKMKTDFTEVQKIESRGDTKQLIKDENSIVKIKNLLDDIKFLQEFVKILVLKDRTIEKDAEFYSELMPYYNELKDIIPLYNKTRNYLTQKPYSTEKIKLNFECPTLLRGWDLNREEANLGVILLKNEKYYLGIINPYCKKIFKIQEKDSNSENNYKKMEYKLLPGPNKMLPKVFFSKSKIDKFMPSDELLEKYNKGCHKKGKDFDINFCHELIDFYKTSLNKHEDWKKFDFKFKSTSEYNDISEFYKDVEEQGYKIEYSEYSEKYINELVDRGELYLFQIYNKDFSEYSKGRPNLHTMYWKAVFDIENIKNPVYKLNGEAEIFYRKKSLERKITHSANEPVANKNENTIKSGKPTSLFKYDLIKDKRYTVDKFQFHVPITMNFKSEKMFNINQVVNKYLKYNDDINVIGIDRGERNLLYVCVIDKNEKIVYQKSLNEIVNEYKSIKYSTNYHALLNKKEKEREIAREDWKNIENIKELKEGYMSQVIHILVELMRKYNAIIVIEDLNKGFKNSRIKVEKQVYQKFEKMFIDKLNYLVFKDEPKESEGGVLNAYQLTNKFETFNKIGKQSGVLYYIPAWCTSKIDPTTGFINRFYIKYENLDKSKEFINKIDDISYNSLENLFEFDIDYSKFTDRLNETRNKWTLYTNGERIYTYRNDKGEWIDKKIQLTNEFKKLFEKYSINLDNIKNEILEKANIEFFKGNNETLGFIQLFKLMVQMRNSLTGKEEDNLISPVKNSNGKFFNTNEQIEGLPKDADANGAYNIARKGLMLIEQMRNTEDDKLNKIKYNITEKEWLDYVQNRGM

SEQ ID NO: 143 - protein, 1226 aa, synthetic construct
MSNLYRNLHNFYSVQKTLRFELIPQGKTKENMEKEGILKADEHRAEIYSKVKKYCDEYHKLFIDKCLKNIRLNELNKYYELYSVVKKDEKQKEEFIKIQEKMRKQISESFKNDNEYKGLFQKDIINIYLITMYKDDKEKIKDISEFNKFTTYFSGYNRNRENMYSEEEKPTGIAYRLINENLPTFIENFKIYNKVIKFMPEIINKIHTDLMEYIQVEDIDEIFDINYYNEVLTQKGIECYNIIISGKSKSNGEKIKGLNEYIAEFNQKHNEKIPKLQELYKQILSDTDTASFKFDTIESDEELLNNIESYYTKLLPVFNKINQLFAKFNKYNLDLIFINNDGTLNTISNEIYKDWSYIRNRIGERYDIEYTGKLKKDTEQYSKQKQEYMKKQKQYSLKFLNDSLRDNYLIEYISNYIEQSKIMEKMKTDFTEVQKIESRGDTKQLIKDENSIVKIKNLLDDIKFLQEFVKILVLKDRTIEKDAEFYSELMPYYNELKDIIPLYNKTRNYLTQKPYSTEKIKLNFECPTLLNGWDLNREEANLGVILLKNEKYYLGIINPYCKKIFKIQEKDSNSENNYKKMEYKLLPGPNKMLPKVFFSKSKIDKFMPSDELLEKYNKGCHKKGKDFDINFCHELIDFYKTSLNKHEDWKKFDFKFKSTSEYNDISEFYKDVEEQGYKIEYSEYSEKYINELVDRGELYLFQIYNKDFSEYSKGRPNLHTMYWKAVFDIENIKNPVYKLNGEAEIFYRKKSLERKITHSANEPVANKNENTIKSGKPTSLFKYDLIKDKRYTVDKFQFHVPITMNFKSEKMFNINQVVNKYLKYNDDINVIGIDRGERNLLYVCVIDKNEKIVYQKSLNEIVNEYKSIKYSTNYHALLNKKEKEREIAREDWKNIENIKELKEGYMSQVIHILVELMRKYNAIIVIEDLNKGFKNSRIKVEKQVYQKFEKMFIDKLNYLVFKDEPKESEGGVLNAYQLTNKFETFNKIGKQSGVLYYIPAWCTSKIDPTTGFINRFYIKYENLDKSKEFINKIDDISYNSLENLFEFDIDYSKFTDRLNETRNKWTLYTNGERIYTYRNDKGEWIDKKIQLTNEFKKLFEKYSINLDNIKNEILEKANIEFFKGNNETLGFIQLFKLMVQMRNSLTGKEEDNLISPVKNSNGKFFNTNEQIEGLPKDADANGAYNIARKGLMLIEQMRNTEDDKLNKIKYNITEKEWLDYVQNRGM

SEQ ID NO: 144 - protein, 1226 aa, synthetic construct
MSNLYRNLHNFYSVQKTLRFELIPQGKTKENMEKEGILKADEHRAEIYSKVKKYCDEYHKLFIDKCLKNIRLNELNKYYELYSVVKKDEKQKEEFIKIQEKMRKQISESFKNDNEYKGLFQKDIINIYLITMYKDDKEKIKDISEFNKFTTYFSGYNKNRENMYSEEEKPTGIAYRLINENLPTFIENFKIYNKVIKFMPEIINKIHTDLMEYIQVEDIDEIFDINYYNEVLTQKGIECYNIIISGKSKSNGEKIKGLNEYINEFNQKHNEKIPKLQELYKQILSDTDTASFKFDTIESDEELLNNIESYYTKLLPVFNKINQLFAKFNKYNLDLIFINNDGTLNTISNEIYKDWSYIRNRIGERYDIEYTGKLKKDTEQYSKQKQEYMKKQKQYSLKFLNDSLRDNYLIEYISNYIEQSKIMEKMKTDFTEVQKIESRGDTKQLIKDENSIVKIKNLLDDIKFLQEFVKILVLKDRTIEKDAEFYSELMPYYNELKDIIPLYNKTRNYLTQKPYSTEKIKLNFERPTLLNGWDLNKEEANLGVILLKNEKYYLGIINPYCKKIFKIQEKDSNSENNYKKMEYKLLPGPNKMLPKVFFSKSKIDKFMPSDELLEKYNKGCHKKGKDFDINFCHELIDFYKTSLNKHEDWKKFDFKFKSTSEYNDISEFYKDVEEQGYKIEYSEYSEKYINELVDRGELYLFQIYNKDFSEYSKGRPNLHTMYWKAVFDIENIKNPVYKLNGEAEIFYRKKSLERKITHSANEPVANKNENTIKSGKPTSLFKYDLIKDKRYTVDKFQFHVPITMNFKSEKMFNINQVVNKYLKYNDDINVIGIDRGERNLLYVCVIDKNEKIVYQKSLNEIVNEYKSIKYSTNYHALLNKKEKEREIAREDWKNIENIKELKEGYMSQVIHILVELMRKYNAIIVIEDLNKGFKNSRIKVEKQVYQKFEKMFIDKLNYLVFKDEPKESEGGVLNAYQLTNKFETFNKIGKQSGVLYYIPAWCTSKIDPTTGFINRFYIKYENLDKSKEFINKIDDISYNSLENLFEFDIDYSKFTDRLNETRNKWTLYTNGERIYTYRNDKGEWIDKKIQLTNEFKKLFEKYSINLDNIKNEILEKANIEFFKGNNETLGFIQLFKLMVQMRNSLTGKEEDNLISPVKNSNGKFFNTNEQIEGLPKDADANGAYNIARKGLMLIEQMRNTEDDKLNKIKYNITEKEWLDYVQNRGM

SEQ ID NO: 145 - protein, 1226 aa, synthetic construct
MNIYKNLHKKYDLSKTLRFELKPIGNTLTNIEEGGFIKKDEQRAIDYKKVKKYCDEYHKVFIERVLSNYELPNELLIKYNELYEKENKDIDTRNEIREIEGKLRKDISEAFKSDEQYKGIFSKDMIQKYLKELFKEQDDKIKEISEFDKFTTYFAGFNRNRENLYIEDEASTSIAHRLINENLPTFITNMKIYKKFTKLFPELKDLISSNLKVNNVEEMFELEGYNKVLSQTGIEHYNLLIAGKSEEKTKIKGINEYINEFNQKNKEYKIPKLKPLYKQILSDKTSESFAFESIDDDSELTEQLEIYNKEFFDAINGNSGIIQLLANLKQYQLDKIYINNNLDLTRISQEIFGDWNRINEILIEDYDKNYTGKVKVNTEKYEDERKKKLKSIEEYSIESLDKKIKEYRDEENKMVDYLETYITLEKVKNIANKYSECQKYLINTSVGEKKLVKNESAINAIKDYLDSVKDIQSFLKMFIPKHKTIETDSNFYNIFSEKYEIIYNIISLYNKTRNYLTQKPYSIEKIKINFNNPILLNGWDLNKEKDCLGVILIKDNKYYLGIINPYNKKIFDIEETKDEENVYKKMEYKLLPGANKMLPKVFLSAKGIENFKPGSEIIEGYSKGLHKKGENFNIEFCHKLIDFFKDSINKNEDWNKFEFKFSPTESYNDISEFYKEIEKQGYNIKFKKYSAEYIEKLVEIGDLFLFEIYNKDFSKFSKGKPNLHTLYWKALFSEKNLENIIYKLNGQAEIFYRKASLNIADTAVHPKNQPIKNKNDLNEKKESIFTYDLIKNKRYTVDKFQFHVPITMNFISEKVNNINEAVNKELKMADHFNIIGIDRGERNLLYITVINEKGEILEQYSLNKIINEYNGKKYVTNYHDLLDAKEKERELARESWKTIENIKELKEGYLSQVIHKITELCRKYKAIIVIEDLNSGFKNSRIKVEKQVYQKFEKMLIDKLNYLIFKDEPEDAEGGVYNAYQLTNKFESFTKMGKQSGILFYIPAWCTSKIDPTTGFVNLFKIKYKNINDAKVFINKFDNIRYNEEDDLYEFDFNYKNFTNKPFGIKENWTICTYGNRIKTFRNKDKNSNWDNQEINLTEEFKKLFSEYNIDNTNLKEDILNQEEKAFFEDLLALIRLTLQMRNSKTGTDIDYMISPVRNKFGEFYNSNMCSEQWPKDADANGAFNIARKGLMLVKQIKDTDVSKLARVKFNITNKEWLEFVQTMEK

SEQ ID NO: 146 - protein, 1226 aa, synthetic construct
MNIYKNLHKKYDLSKTLRFELKPIGNTLTNIEEGGFIKKDEQRAIDYKKVKKYCDEYHKVFIERVLSNYELPNELLIKYNELYEKENKDIDTRNEIREIEGKLRKDISEAFKSDEQYKGIFSKDMIQKYLKELFKEQDDKIKEISEFDKFTTYFAGFNKNRENLYIEDEASTSIAHRLINENLPTFITNMKIYKKFTKLFPELKDLISSNLKVNNVEEMFELEGYNKVLSQTGIEHYNLLIAGKSEEKTKIKGINEYINEFNQKNKEYKIPKLKPLYKQILSDKTSESFAFESIDDDSELTEQLEIYNKEFFDAINGNSGIIQLLANLKQYQLDKIYINNNLDLTRISQEIFGDWNRINEILIEDYDKNYTGKVKVNTEKYEDERKKKLKSIEEYSIESLDKKIKEYRDEENKMVDYLETYITLEKVKNIANKYSECQKYLINTSVGEKKLVKNESAINAIKDYLDSVKDIQSFLKMFIPKHKTIETDSNFYNIFSEKYEIIYNIISLYNKTRNYLTQKPYSIEKIKINFNNPILLRGWDLNKEKDCLGVILIKDNKYYLGIINPYNKKIFDIEETKDEENVYKKMEYKLLPGANKMLPKVFLSAKGIENFKPGSEIIEGYSKGLHKKGENFNIEFCHKLIDFFKDSINKNEDWNKFEFKFSPTESYNDISEFYKEIEKQGYNIKFKKYSAEYIEKLVEIGDLFLFEIYNKDFSKFSKGKPNLHTLYWKALFSEKNLENIIYKLNGQAEIFYRKASLNIADTAVHPKNQPIKNKNDLNEKKESIFTYDLIKNKRYTVDKFQFHVPITMNFISEKVNNINEAVNKELKMADHFNIIGIDRGERNLLYITVINEKGEILEQYSLNKIINEYNGKKYVTNYHDLLDAKEKERELARESWKTIENIKELKEGYLSQVIHKITELCRKYKAIIVIEDLNSGFKNSRIKVEKQVYQKFEKMLIDKLNYLIFKDEPEDAEGGVYNAYQLTNKFESFTKMGKQSGILFYIPAWCTSKIDPTTGFVNLFKIKYKNINDAKVFINKFDNIRYNEEDDLYEFDFNYKNFTNKPFGIKENWTICTYGNRIKTFRNKDKNSNWDNQEINLTEEFKKLFSEYNIDNTNLKEDILNQEEKAFFEDLLALIRLTLQMRNSKTGTDIDYMISPVRNKFGEFYNSNMCSEQWPKDADANGAFNIARKGLMLVKQIKDTDVSKLARVKFNITNKEWLEFVQTMEK

SEQ ID NO: 147 - protein, 1226 aa, synthetic construct
MNIYKNLHKKYDLSKTLRFELKPIGNTLTNIEEGGFIKKDEQRAIDYKKVKKYCDEYHKVFIERVLSNYELPNELLIKYNELYEKENKDIDTRNEIREIEGKLRKDISEAFKSDEQYKGIFSKDMIQKYLKELFKEQDDKIKEISEFDKFTTYFAGFNKNRENLYIEDEASTSIAHRLINENLPTFITNMKIYKKFTKLFPELKDLISSNLKVNNVEEMFELEGYNKVLSQTGIEHYNLLIAGKSEEKTKIKGINEYINEFNQKNKEYKIPKLKPLYKQILSDKTSESFAFESIDDDSELTEQLEIYNKEFFDAINGNSGIIQLLANLKQYQLDKIYINNNLDLTRISQEIFGDWNRINEILIEDYDKNYTGKVKVNTEKYEDERKKKLKSIEEYSIESLDKKIKEYRDEENKMVDYLETYITLEKVKNIANKYSECQKYLINTSVGEKKLVKNESAINAIKDYLDSVKDIQSFLKMFIPKHKTIETDSNFYNIFSEKYEIIYNIISLYNKTRNYLTQKPYSIEKIKINFNNPILLNGWDLNREKDCLGVILIKDNKYYLGIINPYNKKIFDIEETKDEENVYKKMEYKLLPGANKMLPKVFLSAKGIENFKPGSEIIEGYSKGLHKKGENFNIEFCHKLIDFFKDSINKNEDWNKFEFKFSPTESYNDISEFYKEIEKQGYNIKFKKYSAEYIEKLVEIGDLFLFEIYNKDFSKFSKGKPNLHTLYWKALFSEKNLENIIYKLNGQAEIFYRKASLNIADTAVHPKNQPIKNKNDLNEKKESIFTYDLIKNKRYTVDKFQFHVPITMNFISEKVNNINEAVNKELKMADHFNIIGIDRGERNLLYITVINEKGEILEQYSLNKIINEYNGKKYVTNYHDLLDAKEKERELARESWKTIENIKELKEGYLSQVIHKITELCRKYKAIIVIEDLNSGFKNSRIKVEKQVYQKFEKMLIDKLNYLIFKDEPEDAEGGVYNAYQLTNKFESFTKMGKQSGILFYIPAWCTSKIDPTTGFVNLFKIKYKNINDAKVFINKFDNIRYNEEDDLYEFDFNYKNFTNKPFGIKENWTICTYGNRIKTFRNKDKNSNWDNQEINLTEEFKKLFSEYNIDNTNLKEDILNQEEKAFFEDLLALIRLTLQMRNSKTGTDIDYMISPVRNKFGEFYNSNMCSEQWPKDADANGAFNIARKGLMLVKQIKDTDVSKLARVKFNITNKEWLEFVQTMEK

SEQ ID NO: 148 - protein, 1226 aa, synthetic construct
MNIYKNLHKKYDLSKTLRFELKPIGNTLTNIEEGGFIKKDEQRAIDYKKVKKYCDEYHKVFIERVLSNYELPNELLIKYNELYEKENKDIDTRNEIREIEGKLRKDISEAFKSDEQYKGIFSKDMIQKYLKELFKEQDDKIKEISEFDKFTTYFAGFNRNRENLYIEDEASTSIAHRLINENLPTFITNMKIYKKFTKLFPELKDLISSNLKVNNVEEMFELEGYNKVLSQTGIEHYNLLIAGKSEEKTKIKGINEYINEFNQKNKEYKIPKLKPLYKQILSDKTSESFAFESIDDDSELTEQLEIYNKEFFDAINGNSGIIQLLANLKQYQLDKIYINNNLDLTRISQEIFGDWNRINEILIEDYDKNYTGKVKVNTEKYEDERKKKLKSIEEYSIESLDKKIKEYRDEENKMVDYLETYITLEKVKNIANKYSECQKYLINTSVGEKKLVKNESAINAIKDYLDSVKDIQSFLKMFIPKHKTIETDSNFYNIFSEKYEIIYNIISLYNKTRNYLTQKPYSIEKIKINFNNPILLRGWDLNKEKDCLGVILIKDNKYYLGIINPYNKKIFDIEETKDEENVYKKMEYKLLPGANKMLPKVFLSAKGIENFKPGSEIIEGYSKGLHKKGENFNIEFCHKLIDFFKDSINKNEDWNKFEFKFSPTESYNDISEFYKEIEKQGYNIKFKKYSAEYIEKLVEIGDLFLFEIYNKDFSKFSKGKPNLHTLYWKALFSEKNLENIIYKLNGQAEIFYRKASLNIADTAVHPKNQPIKNKNDLNEKKESIFTYDLIKNKRYTVDKFQFHVPITMNFISEKVNNINEAVNKELKMADHFNIIGIDRGERNLLYITVINEKGEILEQYSLNKIINEYNGKKYVTNYHDLLDAKEKERELARESWKTIENIKELKEGYLSQVIHKITELCRKYKAIIVIEDLNSGFKNSRIKVEKQVYQKFEKMLIDKLNYLIFKDEPEDAEGGVYNAYQLTNKFESFTKMGKQSGILFYIPAWCTSKIDPTTGFVNLFKIKYKNINDAKVFINKFDNIRYNEEDDLYEFDFNYKNFTNKPFGIKENWTICTYGNRIKTFRNKDKNSNWDNQEINLTEEFKKLFSEYNIDNTNLKEDILNQEEKAFFEDLLALIRLTLQMRNSKTGTDIDYMISPVRNKFGEFYNSNMCSEQWPKDADANGAFNIARKGLMLVKQIKDTDVSKLARVKFNITNKEWLEFVQTMEK

SEQ ID NO: 149 - protein, 1226 aa, synthetic construct
MNIYKNLHKKYDLSKTLRFELKPIGNTLTNIEEGGFIKKDEQRAIDYKKVKKYCDEYHKVFIERVLSNYELPNELLIKYNELYEKENKDIDTRNEIREIEGKLRKDISEAFKSDEQYKGIFSKDMIQKYLKELFKEQDDKIKEISEFDKFTTYFAGFNRNRENLYIEDEASTSIAHRLINENLPTFITNMKIYKKFTKLFPELKDLISSNLKVNNVEEMFELEGYNKVLSQTGIEHYNLLIAGKSEEKTKIKGINEYINEFNQKNKEYKIPKLKPLYKQILSDKTSESFAFESIDDDSELTEQLEIYNKEFFDAINGNSGIIQLLANLKQYQLDKIYINNNLDLTRISQEIFGDWNRINEILIEDYDKNYTGKVKVNTEKYEDERKKKLKSIEEYSIESLDKKIKEYRDEENKMVDYLETYITLEKVKNIANKYSECQKYLINTSVGEKKLVKNESAINAIKDYLDSVKDIQSFLKMFIPKHKTIETDSNFYNIFSEKYEIIYNIISLYNKTRNYLTQKPYSIEKIKINFNNPILLNGWDLNREKDCLGVILIKDNKYYLGIINPYNKKIFDIEETKDEENVYKKMEYKLLPGANKMLPKVFLSAKGIENFKPGSEIIEGYSKGLHKKGENFNIEFCHKLIDFFKDSINKNEDWNKFEFKFSPTESYNDISEFYKEIEKQGYNIKFKKYSAEYIEKLVEIGDLFLFEIYNKDFSKFSKGKPNLHTLYWKALFSEKNLENIIYKLNGQAEIFYRKASLNIADTAVHPKNQPIKNKNDLNEKKESIFTYDLIKNKRYTVDKFQFHVPITMNFISEKVNNINEAVNKELKMADHFNIIGIDRGERNLLYITVINEKGEILEQYSLNKIINEYNGKKYVTNYHDLLDAKEKERELARESWKTIENIKELKEGYLSQVIHKITELCRKYKAIIVIEDLNSGFKNSRIKVEKQVYQKFEKMLIDKLNYLIFKDEPEDAEGGVYNAYQLTNKFESFTKMGKQSGILFYIPAWCTSKIDPTTGFVNLFKIKYKNINDAKVFINKFDNIRYNEEDDLYEFDFNYKNFTNKPFGIKENWTICTYGNRIKTFRNKDKNSNWDNQEINLTEEFKKLFSEYNIDNTNLKEDILNQEEKAFFEDLLALIRLTLQMRNSKTGTDIDYMISPVRNKFGEFYNSNMCSEQWPKDADANGAFNIARKGLMLVKQIKDTDVSKLARVKFNITNKEWLEFVQTMEK

SEQ ID NO: 150 - protein, 1226 aa, synthetic construct
MNIYKNLHKKYDLSKTLRFELKPIGNTLTNIEEGGFIKKDEQRAIDYKKVKKYCDEYHKVFIERVLSNYELPNELLIKYNELYEKENKDIDTRNEIREIEGKLRKDISEAFKSDEQYKGIFSKDMIQKYLKELFKEQDDKIKEISEFDKFTTYFAGFNKNRENLYIEDEASTSIAHRLINENLPTFITNMKIYKKFTKLFPELKDLISSNLKVNNVEEMFELEGYNKVLSQTGIEHYNLLIAGKSEEKTKIKGINEYINEFNQKNKEYKIPKLKPLYKQILSDKTSESFAFESIDDDSELTEQLEIYNKEFFDAINGNSGIIQLLANLKQYQLDKIYINNNLDLTRISQEIFGDWNRINEILIEDYDKNYTGKVKVNTEKYEDERKKKLKSIEEYSIESLDKKIKEYRDEENKMVDYLETYITLEKVKNIANKYSECQKYLINTSVGEKKLVKNESAINAIKDYLDSVKDIQSFLKMFIPKHKTIETDSNFYNIFSEKYEIIYNIISLYNKTRNYLTQKPYSIEKIKINFNNPILLRGWDLNREKDCLGVILIKDNKYYLGIINPYNKKIFDIEETKDEENVYKKMEYKLLPGANKMLPKVFLSAKGIENFKPGSEIIEGYSKGLHKKGENFNIEFCHKLIDFFKDSINKNEDWNKFEFKFSPTESYNDISEFYKEIEKQGYNIKFKKYSAEYIEKLVEIGDLFLFEIYNKDFSKFSKGKPNLHTLYWKALFSEKNLENIIYKLNGQAEIFYRKASLNIADTAVHPKNQPIKNKNDLNEKKESIFTYDLIKNKRYTVDKFQFHVPITMNFISEKVNNINEAVNKELKMADHFNIIGIDRGERNLLYITVINEKGEILEQYSLNKIINEYNGKKYVTNYHDLLDAKEKERELARESWKTIENIKELKEGYLSQVIHKITELCRKYKAIIVIEDLNSGFKNSRIKVEKQVYQKFEKMLIDKLNYLIFKDEPEDAEGGVYNAYQLTNKFESFTKMGKQSGILFYIPAWCTSKIDPTTGFVNLFKIKYKNINDAKVFINKFDNIRYNEEDDLYEFDFNYKNFTNKPFGIKENWTICTYGNRIKTFRNKDKNSNWDNQEINLTEEFKKLFSEYNIDNTNLKEDILNQEEKAFFEDLLALIRLTLQMRNSKTGTDIDYMISPVRNKFGEFYNSNMCSEQWPKDADANGAFNIARKGLMLVKQIKDTDVSKLARVKFNITNKEWLEFVQTMEK

SEQ ID NO: 151 - protein, 1226 aa, synthetic construct
MNIYKNLHKKYDLSKTLRFELKPIGNTLTNIEEGGFIKKDEQRAIDYKKVKKYCDEYHKVFIERVLSNYELPNELLIKYNELYEKENKDIDTRNEIREIEGKLRKDISEAFKSDEQYKGIFSKDMIQKYLKELFKEQDDKIKEISEFDKFTTYFAGFNRNRENLYIEDEASTSIAHRLINENLPTFITNMKIYKKFTKLFPELKDLISSNLKVNNVEEMFELEGYNKVLSQTGIEHYNLLIAGKSEEKTKIKGINEYINEFNQKNKEYKIPKLKPLYKQILSDKTSESFAFESIDDDSELTEQLEIYNKEFFDAINGNSGIIQLLANLKQYQLDKIYINNNLDLTRISQEIFGDWNRINEILIEDYDKNYTGKVKVNTEKYEDERKKKLKSIEEYSIESLDKKIKEYRDEENKMVDYLETYITLEKVKNIANKYSECQKYLINTSVGEKKLVKNESAINAIKDYLDSVKDIQSFLKMFIPKHKTIETDSNFYNIFSEKYEIIYNIISLYNKTRNYLTQKPYSIEKIKINFNNPILLRGWDLNREKDCLGVILIKDNKYYLGIINPYNKKIFDIEETKDEENVYKKMEYKLLPGANKMLPKVFLSAKGIENFKPGSEIIEGYSKGLHKKGENFNIEFCHKLIDFFKDSINKNEDWNKFEFKFSPTESYNDISEFYKEIEKQGYNIKFKKYSAEYIEKLVEIGDLFLFEIYNKDFSKFSKGKPNLHTLYWKALFSEKNLENIIYKLNGQAEIFYRKASLNIADTAVHPKNQPIKNKNDLNEKKESIFTYDLIKNKRYTVDKFQFHVPITMNFISEKVNNINEAVNKELKMADHFNIIGIDRGERNLLYITVINEKGEILEQYSLNKIINEYNGKKYVTNYHDLLDAKEKERELARESWKTIENIKELKEGYLSQVIHKITELCRKYKAIIVIEDLNSGFKNSRIKVEKQVYQKFEKMLIDKLNYLIFKDEPEDAEGGVYNAYQLTNKFESFTKMGKQSGILFYIPAWCTSKIDPTTGFVNLFKIKYKNINDAKVFINKFDNIRYNEEDDLYEFDFNYKNFTNKPFGIKENWTICTYGNRIKTFRNKDKNSNWDNQEINLTEEFKKLFSEYNIDNTNLKEDILNQEEKAFFEDLLALIRLTLQMRNSKTGTDIDYMISPVRNKFGEFYNSNMCSEQWPKDADANGAFNIARKGLMLVKQIKDTDVSKLARVKFNITNKEWLEFVQTMEK

SEQ ID NO: 152 - protein, 1226 aa, synthetic construct
MNIYKNLHKKYDLSKTLRFELKPIGNTLTNIEEGGFIKKDEQRAIDYKKVKKYCDEYHKVFIERVLSNYELPNELLIKYNELYEKENKDIDTRNEIREIEGKLRKDISEAFKSDEQYKGIFSKDMIQKYLKELFKEQDDKIKEISEFDKFTTYFAGFNRNRENLYIEDEASTSIAHRLINENLPTFITNMKIYKKFTKLFPELKDLISSNLKVNNVEEMFELEGYNKVLSQTGIEHYNLLIAGKSEEKTKIKGINEYIAEFNQKNKEYKIPKLKPLYKQILSDKTSESFAFESIDDDSELTEQLEIYNKEFFDAINGNSGIIQLLANLKQYQLDKIYINNNLDLTRISQEIFGDWNRINEILIEDYDKNYTGKVKVNTEKYEDERKKKLKSIEEYSIESLDKKIKEYRDEENKMVDYLETYITLEKVKNIANKYSECQKYLINTSVGEKKLVKNESAINAIKDYLDSVKDIQSFLKMFIPKHKTIETDSNFYNIFSEKYEIIYNIISLYNKTRNYLTQKPYSIEKIKINFNNPILLNGWDLNREKDCLGVILIKDNKYYLGIINPYNKKIFDIEETKDEENVYKKMEYKLLPGANKMLPKVFLSAKGIENFKPGSEIIEGYSKGLHKKGENFNIEFCHKLIDFFKDSINKNEDWNKFEFKFSPTESYNDISEFYKEIEKQGYNIKFKKYSAEYIEKLVEIGDLFLFEIYNKDFSKFSKGKPNLHTLYWKALFSEKNLENIIYKLNGQAEIFYRKASLNIADTAVHPKNQPIKNKNDLNEKKESIFTYDLIKNKRYTVDKFQFHVPITMNFISEKVNNINEAVNKELKMADHFNIIGIDRGERNLLYITVINEKGEILEQYSLNKIINEYNGKKYVTNYHDLLDAKEKERELARESWKTIENIKELKEGYLSQVIHKITELCRKYKAIIVIEDLNSGFKNSRIKVEKQVYQKFEKMLIDKLNYLIFKDEPEDAEGGVYNAYQLTNKFESFTKMGKQSGILFYIPAWCTSKIDPTTGFVNLFKIKYKNINDAKVFINKFDNIRYNEEDDLYEFDFNYKNFTNKPFGIKENWTICTYGNRIKTFRNKDKNSNWDNQEINLTEEFKKLFSEYNIDNTNLKEDILNQEEKAFFEDLLALIRLTLQMRNSKTGTDIDYMISPVRNKFGEFYNSNMCSEQWPKDADANGAFNIARKGLMLVKQIKDTDVSKLARVKFNITNKEWLEFVQTMEK

SEQ ID NO: 153 - protein, 1226 aa, synthetic construct
MNIYKNLHKKYDLSKTLRFELKPIGNTLTNIEEGGFIKKDEQRAIDYKKVKKYCDEYHKVFIERVLSNYELPNELLIKYNELYEKENKDIDTRNEIREIEGKLRKDISEAFKSDEQYKGIFSKDMIQKYLKELFKEQDDKIKEISEFDKFTTYFAGFNKNRENLYIEDEASTSIAHRLINENLPTFITNMKIYKKFTKLFPELKDLISSNLKVNNVEEMFELEGYNKVLSQTGIEHYNLLIAGKSEEKTKIKGINEYINEFNQKNKEYKIPKLKPLYKQILSDKTSESFAFESIDDDSELTEQLEIYNKEFFDAINGNSGIIQLLANLKQYQLDKIYINNNLDLTRISQEIFGDWNRINEILIEDYDKNYTGKVKVNTEKYEDERKKKLKSIEEYSIESLDKKIKEYRDEENKMVDYLETYITLEKVKNIANKYSECQKYLINTSVGEKKLVKNESAINAIKDYLDSVKDIQSFLKMFIPKHKTIETDSNFYNIFSEKYEIIYNIISLYNKTRNYLTQKPYSIEKIKINFNRPILLNGWDLNKEKDCLGVILIKDNKYYLGIINPYNKKIFDIEETKDEENVYKKMEYKLLPGANKMLPKVFLSAKGIENFKPGSEIIEGYSKGLHKKGENFNIEFCHKLIDFFKDSINKNEDWNKFEFKFSPTESYNDISEFYKEIEKQGYNIKFKKYSAEYIEKLVEIGDLFLFEIYNKDFSKFSKGKPNLHTLYWKALFSEKNLENIIYKLNGQAEIFYRKASLNIADTAVHPKNQPIKNKNDLNEKKESIFTYDLIKNKRYTVDKFLFHVPITMNFISEKVNNINEAVNKELKMADHFNIIGIDRGERNLLYITVINEKGEILEQYSLNKIINEYNGKKYVTNYHDLLDAKEKERELARESWKTIENIKELKEGYLSQVIHKITELCRKYKAIIVIEDLNSGFKNSRIKVEKQVYQKFEKMLIDKLNYLIFKDEPEDAEGGVYNAYQLTNKFESFTKMGKQSGILFYIPAWCTSKIDPTTGFVNLFKIKYKNINDAKVFINKFDNIRYNEEDDLYEFDFNYKNFTNKPFGIKENWTICTYGNRIKTFRNKDKNSNWDNQEINLTEEFKKLFSEYNIDNTNLKEDILNQEEKAFFEDLLALIRLTLQMRNSKTGTDIDYMISPVRNKFGEFYNSNMCSEQWPKDADANGAFNIARKGLMLVKQIKDTDVSKLARVKFNITNKEWLEFVQTMEK

SEQ ID NO: 154 - protein, 1228 aa, synthetic construct
MRQKYDGFESCYGISKVIRTGLIPQGATLEHIEKKGLLLLEAKRAEDYTKVKKIIDRYHRKYIDNQLKDAELSGVEESYSLYCKTNRTEKENTDLKKDLEKLRKQVSNFLTSSAEFKRIDKKELIEKDLLNFVQGDEEEEELVKSFKGFTTYFKGFHRNRKNMYSHEEKSTAIAYRVINQNMFRYFDNIRSISKFILANSEKNIAELESIYSDKLNGKTLKEFFSVDNYSFFISQSGIDTYNMIIGGYSVDERTKVKGINEYINLFNQENHEKFGTLKPLYKQILAERTTESFLPEKFENDKEVIESLAAFSHNMSDTLLEIMEVFGKIGEYDASGIYLKYDSLKDISTCVFSDWSAITNALWERYDVQYIGKKKKETKAYIEEKRKHFKNLKSVSADEINTAMGENVLIPYSDAAKSLFEKICVAEENIRELINLDYPDDKELSKDKAAVKDIKAYLDSVKALQHHMKPLLGNQDESGKNEFFYGDFSGLWDELDALTPLYNKVRNYVTKKPYSTEKIKLNFDNSTLLNGWDLNKEEANTAILMQREGFLYLGIMDKKHNKIFRNIENTGCEDCYQKMEYKLLPGANKMLPKVFFSKKGIESFKPPLEILEIYKNETFKKGDAFNIDDCHTIIDFYKKALNEHEDWKKFEFKFSKTKDYRDISEFYREVEAQGYKISFKAIPKKIIDEYVEKGWLYLFKIWNKDFSEYSKGTPNLHTLYLKMLFDPRNLENVVYKLNGEAEIFYRKASIQPKDIIKHPANEAIKNKNPLNKKQSSIFKYDLIKDRRYTVDKFEFHVPITLNFKARGKENINTEVREYIKRSKNLYVIGVDRGERNLLYVSVVDADGSIVEQYSLNDIVNEYKGVQYRTDYHALLDTKEKNRDKARKEWTEIQNIKELKEGYISQVVHKICELMMKYEAIIALEDLNGGFKNSRKKVEKQVYQKFEKMLIEKLNYLVDKKADVNEPSGLLHALQLTSKFEGFNKLFGQSGFVFYVPAWNTSNIDPCTGFTNLFFSKDLRYASVENSLEFWKKFDSVAYDDTKDIFSFTFDYNNFNQRAEEIKSKSKWTVYSYGDRLETFRNADLNQQYDSREVKLTEAFKELFKKYEIPHENCEIKENILNCKERDFHDKLLHLFKLTLQLRNSKTGTMIDYMTSPVMDIDGNFFDSRTASSSLPQDADANGAYNIARKGLWLIEKLKNTKDADLPKAKLAIKNAEWLAYAQRNENNG

SEQ ID NO: 155 - protein, 1228 aa, synthetic construct
MRQKYDGFESCYGISKVIRTGLIPQGATLEHIEKKGLLLLEAKRAEDYTKVKKIIDRYHRKYIDNQLKDAELSGVEESYSLYCKTNRTEKENTDLKKDLEKLRKQVSNFLTSSAEFKRIDKKELIEKDLLNFVQGDEEEEELVKSFKGFTTYFKGFHENRKNMYSHEEKSTAIAYRVINQNMFRYFDNIRSISKFILANSEKNIAELESIYSDKLNGKTLKEFFSVDNYSFFISQSGIDTYNMIIGGYSVDERTKVKGINEYINLFNQENHEKFGTLKPLYKQILAERTTESFLPEKFENDKEVIESLAAFSHNMSDTLLEIMEVFGKIGEYDASGIYLKYDSLKDISTCVFSDWSAITNALWERYDVQYIGKKKKETKAYIEEKRKHFKNLKSVSADEINTAMGENVLIPYSDAAKSLFEKICVAEENIRELINLDYPDDKELSKDKAAVKDIKAYLDSVKALQHHMKPLLGNQDESGKNEFFYGDFSGLWDELDALTPLYNKVRNYVTKKPYSTEKIKLNFDNSTLLRGWDLNKEEANTAILMQREGFLYLGIMDKKHNKIFRNIENTGCEDCYQKMEYKLLPGANKMLPKVFFSKKGIESFKPPLEILEIYKNETFKKGDAFNIDDCHTIIDFYKKALNEHEDWKKFEFKFSKTKDYRDISEFYREVEAQGYKISFKAIPKKIIDEYVEKGWLYLFKIWNKDFSEYSKGTPNLHTLYLKMLFDPRNLENVVYKLNGEAEIFYRKASIQPKDIIKHPANEAIKNKNPLNKKQSSIFKYDLIKDRRYTVDKFEFHVPITLNFKARGKENINTEVREYIKRSKNLYVIGVDRGERNLLYVSVVDADGSIVEQYSLNDIVNEYKGVQYRTDYHALLDTKEKNRDKARKEWTEIQNIKELKEGYISQVVHKICELMMKYEAIIALEDLNGGFKNSRKKVEKQVYQKFEKMLIEKLNYLVDKKADVNEPSGLLHALQLTSKFEGFNKLFGQSGFVFYVPAWNTSNIDPCTGFTNLFFSKDLRYASVENSLEFWKKFDSVAYDDTKDIFSFTFDYNNFNQRAEEIKSKSKWTVYSYGDRLETFRNADLNQQYDSREVKLTEAFKELFKKYEIPHENCEIKENILNCKERDFHDKLLHLFKLTLQLRNSKTGTMIDYMTSPVMDIDGNFFDSRTASSSLPQDADANGAYNIARKGLWLIEKLKNTKDADLPKAKLAIKNAEWLAYAQRNENNG

SEQ ID NO: 156 - protein, 1228 aa, synthetic construct
MRQKYDGFESCYGISKVIRTGLIPQGATLEHIEKKGLLLLEAKRAEDYTKVKKIIDRYHRKYIDNQLKDAELSGVEESYSLYCKTNRTEKENTDLKKDLEKLRKQVSNFLTSSAEFKRIDKKELIEKDLLNFVQGDEEEEELVKSFKGFTTYFKGFHENRKNMYSHEEKSTAIAYRVINQNMFRYFDNIRSISKFILANSEKNIAELESIYSDKLNGKTLKEFFSVDNYSFFISQSGIDTYNMIIGGYSVDERTKVKGINEYINLFNQENHEKFGTLKPLYKQILAERTTESFLPEKFENDKEVIESLAAFSHNMSDTLLEIMEVFGKIGEYDASGIYLKYDSLKDISTCVFSDWSAITNALWERYDVQYIGKKKKETKAYIEEKRKHFKNLKSVSADEINTAMGENVLIPYSDAAKSLFEKICVAEENIRELINLDYPDDKELSKDKAAVKDIKAYLDSVKALQHHMKPLLGNQDESGKNEFFYGDFSGLWDELDALTPLYNKVRNYVTKKPYSTEKIKLNFDNSTLLNGWDLNREEANTAILMQREGFLYLGIMDKKHNKIFRNIENTGCEDCYQKMEYKLLPGANKMLPKVFFSKKGIESFKPPLEILEIYKNETFKKGDAFNIDDCHTIIDFYKKALNEHEDWKKFEFKFSKTKDYRDISEFYREVEAQGYKISFKAIPKKIIDEYVEKGWLYLFKIWNKDFSEYSKGTPNLHTLYLKMLFDPRNLENVVYKLNGEAEIFYRKASIQPKDIIKHPANEAIKNKNPLNKKQSSIFKYDLIKDRRYTVDKFEFHVPITLNFKARGKENINTEVREYIKRSKNLYVIGVDRGERNLLYVSVVDADGSIVEQYSLNDIVNEYKGVQYRTDYHALLDTKEKNRDKARKEWTEIQNIKELKEGYISQVVHKICELMMKYEAIIALEDLNGGFKNSRKKVEKQVYQKFEKMLIEKLNYLVDKKADVNEPSGLLHALQLTSKFEGFNKLFGQSGFVFYVPAWNTSNIDPCTGFTNLFFSKDLRYASVENSLEFWKKFDSVAYDDTKDIFSFTFDYNNFNQRAEEIKSKSKWTVYSYGDRLETFRNADLNQQYDSREVKLTEAFKELFKKYEIPHENCEIKENILNCKERDFHDKLLHLFKLTLQLRNSKTGTMIDYMTSPVMDIDGNFFDSRTASSSLPQDADANGAYNIARKGLWLIEKLKNTKDADLPKAKLAIKNAEWLAYAQRNENNG

SEQ ID NO: 157 - protein, 1228 aa, synthetic construct
MRQKYDGFESCYGISKVIRTGLIPQGATLEHIEKKGLLLLEAKRAEDYTKVKKIIDRYHRKYIDNQLKDAELSGVEESYSLYCKTNRTEKENTDLKKDLEKLRKQVSNFLTSSAEFKRIDKKELIEKDLLNFVQGDEEEEELVKSFKGFTTYFKGFHRNRKNMYSHEEKSTAIAYRVINQNMFRYFDNIRSISKFILANSEKNIAELESIYSDKLNGKTLKEFFSVDNYSFFISQSGIDTYNMIIGGYSVDERTKVKGINEYINLFNQENHEKFGTLKPLYKQILAERTTESFLPEKFENDKEVIESLAAFSHNMSDTLLEIMEVFGKIGEYDASGIYLKYDSLKDISTCVFSDWSAITNALWERYDVQYIGKKKKETKAYIEEKRKHFKNLKSVSADEINTAMGENVLIPYSDAAKSLFEKICVAEENIRELINLDYPDDKELSKDKAAVKDIKAYLDSVKALQHHMKPLLGNQDESGKNEFFYGDFSGLWDELDALTPLYNKVRNYVTKKPYSTEKIKLNFDNSTLLRGWDLNKEEANTAILMQREGFLYLGIMDKKHNKIFRNIENTGCEDCYQKMEYKLLPGANKMLPKVFFSKKGIESFKPPLEILEIYKNETFKKGDAFNIDDCHTIIDFYKKALNEHEDWKKFEFKFSKTKDYRDISEFYREVEAQGYKISFKAIPKKIIDEYVEKGWLYLFKIWNKDFSEYSKGTPNLHTLYLKMLFDPRNLENVVYKLNGEAEIFYRKASIQPKDIIKHPANEAIKNKNPLNKKQSSIFKYDLIKDRRYTVDKFEFHVPITLNFKARGKENINTEVREYIKRSKNLYVIGVDRGERNLLYVSVVDADGSIVEQYSLNDIVNEYKGVQYRTDYHALLDTKEKNRDKARKEWTEIQNIKELKEGYISQVVHKICELMMKYEAIIALEDLNGGFKNSRKKVEKQVYQKFEKMLIEKLNYLVDKKADVNEPSGLLHALQLTSKFEGFNKLFGQSGFVFYVPAWNTSNIDPCTGFTNLFFSKDLRYASVENSLEFWKKFDSVAYDDTKDIFSFTFDYNNFNQRAEEIKSKSKWTVYSYGDRLETFRNADLNQQYDSREVKLTEAFKELFKKYEIPHENCEIKENILNCKERDFHDKLLHLFKLTLQLRNSKTGTMIDYMTSPVMDIDGNFFDSRTASSSLPQDADANGAYNIARKGLWLIEKLKNTKDADLPKAKLAIKNAEWLAYAQRNENNG

SEQ ID NO: 158 - protein, 1228 aa, synthetic construct
MRQKYDGFESCYGISKVIRTGLIPQGATLEHIEKKGLLLLEAKRAEDYTKVKKIIDRYHRKYIDNQLKDAELSGVEESYSLYCKTNRTEKENTDLKKDLEKLRKQVSNFLTSSAEFKRIDKKELIEKDLLNFVQGDEEEEELVKSFKGFTTYFKGFHRNRKNMYSHEEKSTAIAYRVINQNMFRYFDNIRSISKFILANSEKNIAELESIYSDKLNGKTLKEFFSVDNYSFFISQSGIDTYNMIIGGYSVDERTKVKGINEYINLFNQENHEKFGTLKPLYKQILAERTTESFLPEKFENDKEVIESLAAFSHNMSDTLLEIMEVFGKIGEYDASGIYLKYDSLKDISTCVFSDWSAITNALWERYDVQYIGKKKKETKAYIEEKRKHFKNLKSVSADEINTAMGENVLIPYSDAAKSLFEKICVAEENIRELINLDYPDDKELSKDKAAVKDIKAYLDSVKALQHHMKPLLGNQDESGKNEFFYGDFSGLWDELDALTPLYNKVRNYVTKKPYSTEKIKLNFDNSTLLNGWDLNREEANTAILMQREGFLYLGIMDKKHNKIFRNIENTGCEDCYQKMEYKLLPGANKMLPKVFFSKKGIESFKPPLEILEIYKNETFKKGDAFNIDDCHTIIDFYKKALNEHEDWKKFEFKFSKTKDYRDISEFYREVEAQGYKISFKAIPKKIIDEYVEKGWLYLFKIWNKDFSEYSKGTPNLHTLYLKMLFDPRNLENVVYKLNGEAEIFYRKASIQPKDIIKHPANEAIKNKNPLNKKQSSIFKYDLIKDRRYTVDKFEFHVPITLNFKARGKENINTEVREYIKRSKNLYVIGVDRGERNLLYVSVVDADGSIVEQYSLNDIVNEYKGVQYRTDYHALLDTKEKNRDKARKEWTEIQNIKELKEGYISQVVHKICELMMKYEAIIALEDLNGGFKNSRKKVEKQVYQKFEKMLIEKLNYLVDKKADVNEPSGLLHALQLTSKFEGFNKLFGQSGFVFYVPAWNTSNIDPCTGFTNLFFSKDLRYASVENSLEFWKKFDSVAYDDTKDIFSFTFDYNNFNQRAEEIKSKSKWTVYSYGDRLETFRNADLNQQYDSREVKLTEAFKELFKKYEIPHENCEIKENILNCKERDFHDKLLHLFKLTLQLRNSKTGTMIDYMTSPVMDIDGNFFDSRTASSSLPQDADANGAYNIARKGLWLIEKLKNTKDADLPKAKLAIKNAEWLAYAQRNENNG

SEQ ID NO: 159 - protein, 1228 aa, synthetic construct
MRQKYDGFESCYGISKVIRTGLIPQGATLEHIEKKGLLLLEAKRAEDYTKVKKIIDRYHRKYIDNQLKDAELSGVEESYSLYCKTNRTEKENTDLKKDLEKLRKQVSNFLTSSAEFKRIDKKELIEKDLLNFVQGDEEEEELVKSFKGFTTYFKGFHENRKNMYSHEEKSTAIAYRVINQNMFRYFDNIRSISKFILANSEKNIAELESIYSDKLNGKTLKEFFSVDNYSFFISQSGIDTYNMIIGGYSVDERTKVKGINEYINLFNQENHEKFGTLKPLYKQILAERTTESFLPEKFENDKEVIESLAAFSHNMSDTLLEIMEVFGKIGEYDASGIYLKYDSLKDISTCVFSDWSAITNALWERYDVQYIGKKKKETKAYIEEKRKHFKNLKSVSADEINTAMGENVLIPYSDAAKSLFEKICVAEENIRELINLDYPDDKELSKDKAAVKDIKAYLDSVKALQHHMKPLLGNQDESGKNEFFYGDFSGLWDELDALTPLYNKVRNYVTKKPYSTEKIKLNFDNSTLLRGWDLNREEANTAILMQREGFLYLGIMDKKHNKIFRNIENTGCEDCYQKMEYKLLPGANKMLPKVFFSKKGIESFKPPLEILEIYKNETFKKGDAFNIDDCHTIIDFYKKALNEHEDWKKFEFKFSKTKDYRDISEFYREVEAQGYKISFKAIPKKIIDEYVEKGWLYLFKIWNKDFSEYSKGTPNLHTLYLKMLFDPRNLENVVYKLNGEAEIFYRKASIQPKDIIKHPANEAIKNKNPLNKKQSSIFKYDLIKDRRYTVDKFEFHVPITLNFKARGKENINTEVREYIKRSKNLYVIGVDRGERNLLYVSVVDADGSIVEQYSLNDIVNEYKGVQYRTDYHALLDTKEKNRDKARKEWTEIQNIKELKEGYISQVVHKICELMMKYEAIIALEDLNGGFKNSRKKVEKQVYQKFEKMLIEKLNYLVDKKADVNEPSGLLHALQLTSKFEGFNKLFGQSGFVFYVPAWNTSNIDPCTGFTNLFFSKDLRYASVENSLEFWKKFDSVAYDDTKDIFSFTFDYNNFNQRAEEIKSKSKWTVYSYGDRLETFRNADLNQQYDSREVKLTEAFKELFKKYEIPHENCEIKENILNCKERDFHDKLLHLFKLTLQLRNSKTGTMIDYMTSPVMDIDGNFFDSRTASSSLPQDADANGAYNIARKGLWLIEKLKNTKDADLPKAKLAIKNAEWLAYAQRNENNG

SEQ ID NO: 160 - protein, 1228 aa, synthetic construct
MRQKYDGFESCYGISKVIRTGLIPQGATLEHIEKKGLLLLEAKRAEDYTKVKKIIDRYHRKYIDNQLKDAELSGVEESYSLYCKTNRTEKENTDLKKDLEKLRKQVSNFLTSSAEFKRIDKKELIEKDLLNFVQGDEEEEELVKSFKGFTTYFKGFHRNRKNMYSHEEKSTAIAYRVINQNMFRYFDNIRSISKFILANSEKNIAELESIYSDKLNGKTLKEFFSVDNYSFFISQSGIDTYNMIIGGYSVDERTKVKGINEYINLFNQENHEKFGTLKPLYKQILAERTTESFLPEKFENDKEVIESLAAFSHNMSDTLLEIMEVFGKIGEYDASGIYLKYDSLKDISTCVFSDWSAITNALWERYDVQYIGKKKKETKAYIEEKRKHFKNLKSVSADEINTAMGENVLIPYSDAAKSLFEKICVAEENIRELINLDYPDDKELSKDKAAVKDIKAYLDSVKALQHHMKPLLGNQDESGKNEFFYGDFSGLWDELDALTPLYNKVRNYVTKKPYSTEKIKLNFDNSTLLRGWDLNREEANTAILMQREGFLYLGIMDKKHNKIFRNIENTGCEDCYQKMEYKLLPGANKMLPKVFFSKKGIESFKPPLEILEIYKNETFKKGDAFNIDDCHTIIDFYKKALNEHEDWKKFEFKFSKTKDYRDISEFYREVEAQGYKISFKAIPKKIIDEYVEKGWLYLFKIWNKDFSEYSKGTPNLHTLYLKMLFDPRNLENVVYKLNGEAEIFYRKASIQPKDIIKHPANEAIKNKNPLNKKQSSIFKYDLIKDRRYTVDKFEFHVPITLNFKARGKENINTEVREYIKRSKNLYVIGVDRGERNLLYVSVVDADGSIVEQYSLNDIVNEYKGVQYRTDYHALLDTKEKNRDKARKEWTEIQNIKELKEGYISQVVHKICELMMKYEAIIALEDLNGGFKNSRKKVEKQVYQKFEKMLIEKLNYLVDKKADVNEPSGLLHALQLTSKFEGFNKLFGQSGFVFYVPAWNTSNIDPCTGFTNLFFSKDLRYASVENSLEFWKKFDSVAYDDTKDIFSFTFDYNNFNQRAEEIKSKSKWTVYSYGDRLETFRNADLNQQYDSREVKLTEAFKELFKKYEIPHENCEIKENILNCKERDFHDKLLHLFKLTLQLRNSKTGTMIDYMTSPVMDIDGNFFDSRTASSSLPQDADANGAYNIARKGLWLIEKLKNTKDADLPKAKLAIKNAEWLAYAQRNENNG

SEQ ID NO: 161 - protein, 1228 aa, synthetic construct
MRQKYDGFESCYGISKVIRTGLIPQGATLEHIEKKGLLLLEAKRAEDYTKVKKIIDRYHRKYIDNQLKDAELSGVEESYSLYCKTNRTEKENTDLKKDLEKLRKQVSNFLTSSAEFKRIDKKELIEKDLLNFVQGDEEEEELVKSFKGFTTYFKGFHRNRKNMYSHEEKSTAIAYRVINQNMFRYFDNIRSISKFILANSEKNIAELESIYSDKLNGKTLKEFFSVDNYSFFISQSGIDTYNMIIGGYSVDERTKVKGINEYIALFNQENHEKFGTLKPLYKQILAERTTESFLPEKFENDKEVIESLAAFSHNMSDTLLEIMEVFGKIGEYDASGIYLKYDSLKDISTCVFSDWSAITNALWERYDVQYIGKKKKETKAYIEEKRKHFKNLKSVSADEINTAMGENVLIPYSDAAKSLFEKICVAEENIRELINLDYPDDKELSKDKAAVKDIKAYLDSVKALQHHMKPLLGNQDESGKNEFFYGDFSGLWDELDALTPLYNKVRNYVTKKPYSTEKIKLNFDNSTLLNGWDLNREEANTAILMQREGFLYLGIMDKKHNKIFRNIENTGCEDCYQKMEYKLLPGANKMLPKVFFSKKGIESFKPPLEILEIYKNETFKKGDAFNIDDCHTIIDFYKKALNEHEDWKKFEFKFSKTKDYRDISEFYREVEAQGYKISFKAIPKKIIDEYVEKGWLYLFKIWNKDFSEYSKGTPNLHTLYLKMLFDPRNLENVVYKLNGEAEIFYRKASIQPKDIIKHPANEAIKNKNPLNKKQSSIFKYDLIKDRRYTVDKFEFHVPITLNFKARGKENINTEVREYIKRSKNLYVIGVDRGERNLLYVSVVDADGSIVEQYSLNDIVNEYKGVQYRTDYHALLDTKEKNRDKARKEWTEIQNIKELKEGYISQVVHKICELMMKYEAIIALEDLNGGFKNSRKKVEKQVYQKFEKMLIEKLNYLVDKKADVNEPSGLLHALQLTSKFEGFNKLFGQSGFVFYVPAWNTSNIDPCTGFTNLFFSKDLRYASVENSLEFWKKFDSVAYDDTKDIFSFTFDYNNFNQRAEEIKSKSKWTVYSYGDRLETFRNADLNQQYDSREVKLTEAFKELFKKYEIPHENCEIKENILNCKERDFHDKLLHLFKLTLQLRNSKTGTMIDYMTSPVMDIDGNFFDSRTASSSLPQDADANGAYNIARKGLWLIEKLKNTKDADLPKAKLAIKNAEWLAYAQRNENNG

SEQ ID NO: 162 - protein, 1228 aa, synthetic construct
MRQKYDGFESCYGISKVIRTGLIPQGATLEHIEKKGLLLLEAKRAEDYTKVKKIIDRYHRKYIDNQLKDAELSGVEESYSLYCKTNRTEKENTDLKKDLEKLRKQVSNFLTSSAEFKRIDKKELIEKDLLNFVQGDEEEEELVKSFKGFTTYFKGFHENRKNMYSHEEKSTAIAYRVINQNMFRYFDNIRSISKFILANSEKNIAELESIYSDKLNGKTLKEFFSVDNYSFFISQSGIDTYNMIIGGYSVDERTKVKGINEYINLFNQENHEKFGTLKPLYKQILAERTTESFLPEKFENDKEVIESLAAFSHNMSDTLLEIMEVFGKIGEYDASGIYLKYDSLKDISTCVFSDWSAITNALWERYDVQYIGKKKKETKAYIEEKRKHFKNLKSVSADEINTAMGENVLIPYSDAAKSLFEKICVAEENIRELINLDYPDDKELSKDKAAVKDIKAYLDSVKALQHHMKPLLGNQDESGKNEFFYGDFSGLWDELDALTPLYNKVRNYVTKKPYSTEKIKLNFDRSTLLNGWDLNKEEANTAILMQREGFLYLGIMDKKHNKIFRNIENTGCEDCYQKMEYKLLPGANKMLPKVFFSKKGIESFKPPLEILEIYKNETFKKGDAFNIDDCHTIIDFYKKALNEHEDWKKFEFKFSKTKDYRDISEFYREVEAQGYKISFKAIPKKIIDEYVEKGWLYLFKIWNKDFSEYSKGTPNLHTLYLKMLFDPRNLENVVYKLNGEAEIFYRKASIQPKDIIKHPANEAIKNKNPLNKKQSSIFKYDLIKDRRYTVDKFEFHVPITLNFKARGKENINTEVREYIKRSKNLYVIGVDRGERNLLYVSVVDADGSIVEQYSLNDIVNEYKGVQYRTDYHALLDTKEKNRDKARKEWTEIQNIKELKEGYISQVVHKICELMMKYEAIIALEDLNGGFKNSRKKVEKQVYQKFEKMLIEKLNYLVDKKADVNEPSGLLHALQLTSKFEGFNKLFGQSGFVFYVPAWNTSNIDPCTGFTNLFFSKDLRYASVENSLEFWKKFDSVAYDDTKDIFSFTFDYNNFNQRAEEIKSKSKWTVYSYGDRLETFRNADLNQQYDSREVKLTEAFKELFKKYEIPHENCEIKENILNCKERDFHDKLLHLFKLTLQLRNSKTGTMIDYMTSPVMDIDGNFFDSRTASSSLPQDADANGAYNIARKGLWLIEKLKNTKDADLPKAKLAIKNAEWLAYAQRNENNG

SEQ ID NO: 163 - protein, 1228 aa, synthetic construct
MWEDFTKLYSLSKTLRFELVPTESTEQQLKTSGVIQADKVRHESFKLAKKVLDEIHRKFITDSLVDLQLDHKQLQVFAEYSLNPASKSDAAKQAELIKLRRSIYMAFEQRFTATQAGWKEAYDCEKFISSPKSQLALIETFKPELADKLGMPEQDLEDLQTRFNKFASYFQGYNRNRENIYTTKGHAVEISHRTIDQNLLKFYKNVQKYEKYYTDKDILGGEAAPHFKVENYTSYITQDEINAYNEAIRSFRSNINQARQGLDKKEKQSLPMFEPLFKQILGDNTKKVLFEKLEGKEEYAELTRKLQQDMPSWVTALDEHANTHIMIPANPATVWFNAKAVNAVLHKHTTSPELIKIYLPKEFTSSRRAKDEETAKQNSFVSLRQLFAALDQAQVATELSLLKNVDRADMGASEAVWLAFCEDIQVKIKDARETVGAFSKIIAFENKSEKETLKKALDSFNDLYRMYTYFELRYKNEPVTSFDTDPDFYELFEDPDDQDSLFDADKDKRFSHYYDLIRNYLTKKSYSLDKWKLNFDCSQLLTGWDVNKEPEKHGVILQNGENYHLAILNNESNKIFDRAKNKLLFQAEDNTAWQKMDYKLLPGPNKMLPKCLLPKSNRNKYGATDEVLDIYETGSFKKGDNFNLASLHKMIDFYKQAINRYEGWSMYEFTFRSTDQYQGIDQFYHDAESQGYRLGFVPINSVELQKMVDDGRIYLFRIQNKDLANPSSKTANMHTLYFRELFKSSSTIKLNGEAEIFFRPKTENLAASKTNRKTPQGTEVTELKRFRSDKIQLHLPVNLNYQTKSLINATFNQHTIERIKTNGVNAIIGIDRGEKHLAYVAIVDRSGKLLDEPISLNNITSKLPGGGKKVTPYFDLLDHREKERKENRQNWEAVRQIKDLKSGYIGHCIKQIVDLMIKHNAVVALENLNVGFKQGRSAIEKSVYNQLEQALLHKLQYVVDKSKPPQELFGAQNGVQLAPPSVVPKDTYNHMGFVFFVDPSYTSAVDPKTGYRQHFRLDDRINTKTFNTFIRDGFKDIEYKDSNLLFTFNWRQLASARNKIKKDVRAEKEAEISDKPWTITANIERLVYSKKADDGHGATVKVYPQVELLQLFESKNFDLRKNIKQQLLEHKPDAKFVGSFTYLFNIINKLRNSDAASDVDAIISPVYIDGFNSTKHKLGGYEWNGDANGAYNIARKGAILLDKLYEAKTLRDFKATVTRAEYDAYLTK

SEQ ID NO: 164 - protein, 1228 aa, synthetic construct
MWEDFTKLYSLSKTLRFELVPTESTEQQLKTSGVIQADKVRHESFKLAKKVLDEIHRKFITDSLVDLQLDHKQLQVFAEYSLNPASKSDAAKQAELIKLRRSIYMAFEQRFTATQAGWKEAYDCEKFISSPKSQLALIETFKPELADKLGMPEQDLEDLQTRFNKFASYFQGYNKNRENIYTTKGHAVEISHRTIDQNLLKFYKNVQKYEKYYTDKDILGGEAAPHFKVENYTSYITQDEINAYNEAIRSFRSNINQARQGLDKKEKQSLPMFEPLFKQILGDNTKKVLFEKLEGKEEYAELTRKLQQDMPSWVTALDEHANTHIMIPANPATVWFNAKAVNAVLHKHTTSPELIKIYLPKEFTSSRRAKDEETAKQNSFVSLRQLFAALDQAQVATELSLLKNVDRADMGASEAVWLAFCEDIQVKIKDARETVGAFSKIIAFENKSEKETLKKALDSFNDLYRMYTYFELRYKNEPVTSFDTDPDFYELFEDPDDQDSLFDADKDKRFSHYYDLIRNYLTKKSYSLDKWKLNFDCSQLLRGWDVNKEPEKHGVILQNGENYHLAILNNESNKIFDRAKNKLLFQAEDNTAWQKMDYKLLPGPNKMLPKCLLPKSNRNKYGATDEVLDIYETGSFKKGDNFNLASLHKMIDFYKQAINRYEGWSMYEFTFRSTDQYQGIDQFYHDAESQGYRLGFVPINSVELQKMVDDGRIYLFRIQNKDLANPSSKTANMHTLYFRELFKSSSTIKLNGEAEIFFRPKTENLAASKTNRKTPQGTEVTELKRFRSDKIQLHLPVNLNYQTKSLINATFNQHTIERIKTNGVNAIIGIDRGEKHLAYVAIVDRSGKLLDEPISLNNITSKLPGGGKKVTPYFDLLDHREKERKENRQNWEAVRQIKDLKSGYIGHCIKQIVDLMIKHNAVVALENLNVGFKQGRSAIEKSVYNQLEQALLHKLQYVVDKSKPPQELFGAQNGVQLAPPSVVPKDTYNHMGFVFFVDPSYTSAVDPKTGYRQHFRLDDRINTKTFNTFIRDGFKDIEYKDSNLLFTFNWRQLASARNKIKKDVRAEKEAEISDKPWTITANIERLVYSKKADDGHGATVKVYPQVELLQLFESKNFDLRKNIKQQLLEHKPDAKFVGSFTYLFNIINKLRNSDAASDVDAIISPVYIDGFNSTKHKLGGYEWNGDANGAYNIARKGAILLDKLYEAKTLRDFKATVTRAEYDAYLTK

SEQ ID NO: 165 - protein, 1228 aa, synthetic construct
MWEDFTKLYSLSKTLRFELVPTESTEQQLKTSGVIQADKVRHESFKLAKKVLDEIHRKFITDSLVDLQLDHKQLQVFAEYSLNPASKSDAAKQAELIKLRRSIYMAFEQRFTATQAGWKEAYDCEKFISSPKSQLALIETFKPELADKLGMPEQDLEDLQTRFNKFASYFQGYNKNRENIYTTKGHAVEISHRTIDQNLLKFYKNVQKYEKYYTDKDILGGEAAPHFKVENYTSYITQDEINAYNEAIRSFRSNINQARQGLDKKEKQSLPMFEPLFKQILGDNTKKVLFEKLEGKEEYAELTRKLQQDMPSWVTALDEHANTHIMIPANPATVWFNAKAVNAVLHKHTTSPELIKIYLPKEFTSSRRAKDEETAKQNSFVSLRQLFAALDQAQVATELSLLKNVDRADMGASEAVWLAFCEDIQVKIKDARETVGAFSKIIAFENKSEKETLKKALDSFNDLYRMYTYFELRYKNEPVTSFDTDPDFYELFEDPDDQDSLFDADKDKRFSHYYDLIRNYLTKKSYSLDKWKLNFDCSQLLTGWDVNREPEKHGVILQNGENYHLAILNNESNKIFDRAKNKLLFQAEDNTAWQKMDYKLLPGPNKMLPKCLLPKSNRNKYGATDEVLDIYETGSFKKGDNFNLASLHKMIDFYKQAINRYEGWSMYEFTFRSTDQYQGIDQFYHDAESQGYRLGFVPINSVELQKMVDDGRIYLFRIQNKDLANPSSKTANMHTLYFRELFKSSSTIKLNGEAEIFFRPKTENLAASKTNRKTPQGTEVTELKRFRSDKIQLHLPVNLNYQTKSLINATFNQHTIERIKTNGVNAIIGIDRGEKHLAYVAIVDRSGKLLDEPISLNNITSKLPGGGKKVTPYFDLLDHREKERKENRQNWEAVRQIKDLKSGYIGHCIKQIVDLMIKHNAVVALENLNVGFKQGRSAIEKSVYNQLEQALLHKLQYVVDKSKPPQELFGAQNGVQLAPPSVVPKDTYNHMGFVFFVDPSYTSAVDPKTGYRQHFRLDDRINTKTFNTFIRDGFKDIEYKDSNLLFTFNWRQLASARNKIKKDVRAEKEAEISDKPWTITANIERLVYSKKADDGHGATVKVYPQVELLQLFESKNFDLRKNIKQQLLEHKPDAKFVGSFTYLFNIINKLRNSDAASDVDAIISPVYIDGFNSTKHKLGGYEWNGDANGAYNIARKGAILLDKLYEAKTLRDFKATVTRAEYDAYLTK

SEQ ID NO: 166 - protein, 1228 aa, synthetic construct
MWEDFTKLYSLSKTLRFELVPTESTEQQLKTSGVIQADKVRHESFKLAKKVLDEIHRKFITDSLVDLQLDHKQLQVFAEYSLNPASKSDAAKQAELIKLRRSIYMAFEQRFTATQAGWKEAYDCEKFISSPKSQLALIETFKPELADKLGMPEQDLEDLQTRFNKFASYFQGYNRNRENIYTTKGHAVEISHRTIDQNLLKFYKNVQKYEKYYTDKDILGGEAAPHFKVENYTSYITQDEINAYNEAIRSFRSNINQARQGLDKKEKQSLPMFEPLFKQILGDNTKKVLFEKLEGKEEYAELTRKLQQDMPSWVTALDEHANTHIMIPANPATVWFNAKAVNAVLHKHTTSPELIKIYLPKEFTSSRRAKDEETAKQNSFVSLRQLFAALDQAQVATELSLLKNVDRADMGASEAVWLAFCEDIQVKIKDARETVGAFSKIIAFENKSEKETLKKALDSFNDLYRMYTYFELRYKNEPVTSFDTDPDFYELFEDPDDQDSLFDADKDKRFSHYYDLIRNYLTKKSYSLDKWKLNFDCSQLLRGWDVNKEPEKHGVILQNGENYHLAILNNESNKIFDRAKNKLLFQAEDNTAWQKMDYKLLPGPNKMLPKCLLPKSNRNKYGATDEVLDIYETGSFKKGDNFNLASLHKMIDFYKQAINRYEGWSMYEFTFRSTDQYQGIDQFYHDAESQGYRLGFVPINSVELQKMVDDGRIYLFRIQNKDLANPSSKTANMHTLYFRELFKSSSTIKLNGEAEIFFRPKTENLAASKTNRKTPQGTEVTELKRFRSDKIQLHLPVNLNYQTKSLINATFNQHTIERIKTNGVNAIIGIDRGEKHLAYVAIVDRSGKLLDEPISLNNITSKLPGGGKKVTPYFDLLDHREKERKENRQNWEAVRQIKDLKSGYIGHCIKQIVDLMIKHNAVVALENLNVGFKQGRSAIEKSVYNQLEQALLHKLQYVVDKSKPPQELFGAQNGVQLAPPSVVPKDTYNHMGFVFFVDPSYTSAVDPKTGYRQHFRLDDRINTKTFNTFIRDGFKDIEYKDSNLLFTFNWRQLASARNKIKKDVRAEKEAEISDKPWTITANIERLVYSKKADDGHGATVKVYPQVELLQLFESKNFDLRKNIKQQLLEHKPDAKFVGSFTYLFNIINKLRNSDAASDVDAIISPVYIDGFNSTKHKLGGYEWNGDANGAYNIARKGAILLDKLYEAKTLRDFKATVTRAEYDAYLTK

SEQ ID NO: 167 - protein, 1228 aa, synthetic construct
MWEDFTKLYSLSKTLRFELVPTESTEQQLKTSGVIQADKVRHESFKLAKKVLDEIHRKFITDSLVDLQLDHKQLQVFAEYSLNPASKSDAAKQAELIKLRRSIYMAFEQRFTATQAGWKEAYDCEKFISSPKSQLALIETFKPELADKLGMPEQDLEDLQTRFNKFASYFQGYNRNRENIYTTKGHAVEISHRTIDQNLLKFYKNVQKYEKYYTDKDILGGEAAPHFKVENYTSYITQDEINAYNEAIRSFRSNINQARQGLDKKEKQSLPMFEPLFKQILGDNTKKVLFEKLEGKEEYAELTRKLQQDMPSWVTALDEHANTHIMIPANPATVWFNAKAVNAVLHKHTTSPELIKIYLPKEFTSSRRAKDEETAKQNSFVSLRQLFAALDQAQVATELSLLKNVDRADMGASEAVWLAFCEDIQVKIKDARETVGAFSKIIAFENKSEKETLKKALDSFNDLYRMYTYFELRYKNEPVTSFDTDPDFYELFEDPDDQDSLFDADKDKRFSHYYDLIRNYLTKKSYSLDKWKLNFDCSQLLTGWDVNREPEKHGVILQNGENYHLAILNNESNKIFDRAKNKLLFQAEDNTAWQKMDYKLLPGPNKMLPKCLLPKSNRNKYGATDEVLDIYETGSFKKGDNFNLASLHKMIDFYKQAINRYEGWSMYEFTFRSTDQYQGIDQFYHDAESQGYRLGFVPINSVELQKMVDDGRIYLFRIQNKDLANPSSKTANMHTLYFRELFKSSSTIKLNGEAEIFFRPKTENLAASKTNRKTPQGTEVTELKRFRSDKIQLHLPVNLNYQTKSLINATFNQHTIERIKTNGVNAIIGIDRGEKHLAYVAIVDRSGKLLDEPISLNNITSKLPGGGKKVTPYFDLLDHREKERKENRQNWEAVRQIKDLKSGYIGHCIKQIVDLMIKHNAVVALENLNVGFKQGRSAIEKSVYNQLEQALLHKLQYVVDKSKPPQELFGAQNGVQLAPPSVVPKDTYNHMGFVFFVDPSYTSAVDPKTGYRQHFRLDDRINTKTFNTFIRDGFKDIEYKDSNLLFTFNWRQLASARNKIKKDVRAEKEAEISDKPWTITANIERLVYSKKADDGHGATVKVYPQVELLQLFESKNFDLRKNIKQQLLEHKPDAKFVGSFTYLFNIINKLRNSDAASDVDAIISPVYIDGFNSTKHKLGGYEWNGDANGAYNIARKGAILLDKLYEAKTLRDFKATVTRAEYDAYLTK

SEQ ID NO: 168 - protein, 1228 aa, synthetic construct
MWEDFTKLYSLSKTLRFELVPTESTEQQLKTSGVIQADKVRHESFKLAKKVLDEIHRKFITDSLVDLQLDHKQLQVFAEYSLNPASKSDAAKQAELIKLRRSIYMAFEQRFTATQAGWKEAYDCEKFISSPKSQLALIETFKPELADKLGMPEQDLEDLQTRFNKFASYFQGYNKNRENIYTTKGHAVEISHRTIDQNLLKFYKNVQKYEKYYTDKDILGGEAAPHFKVENYTSYITQDEINAYNEAIRSFRSNINQARQGLDKKEKQSLPMFEPLFKQILGDNTKKVLFEKLEGKEEYAELTRKLQQDMPSWVTALDEHANTHIMIPANPATVWFNAKAVNAVLHKHTTSPELIKIYLPKEFTSSRRAKDEETAKQNSFVSLRQLFAALDQAQVATELSLLKNVDRADMGASEAVWLAFCEDIQVKIKDARETVGAFSKIIAFENKSEKETLKKALDSFNDLYRMYTYFELRYKNEPVTSFDTDPDFYELFEDPDDQDSLFDADKDKRFSHYYDLIRNYLTKKSYSLDKWKLNFDCSQLLRGWDVNREPEKHGVILQNGENYHLAILNNESNKIFDRAKNKLLFQAEDNTAWQKMDYKLLPGPNKMLPKCLLPKSNRNKYGATDEVLDIYETGSFKKGDNFNLASLHKMIDFYKQAINRYEGWSMYEFTFRSTDQYQGIDQFYHDAESQGYRLGFVPINSVELQKMVDDGRIYLFRIQNKDLANPSSKTANMHTLYFRELFKSSSTIKLNGEAEIFFRPKTENLAASKTNRKTPQGTEVTELKRFRSDKIQLHLPVNLNYQTKSLINATFNQHTIERIKTNGVNAIIGIDRGEKHLAYVAIVDRSGKLLDEPISLNNITSKLPGGGKKVTPYFDLLDHREKERKENRQNWEAVRQIKDLKSGYIGHCIKQIVDLMIKHNAVVALENLNVGFKQGRSAIEKSVYNQLEQALLHKLQYVVDKSKPPQELFGAQNGVQLAPPSVVPKDTYNHMGFVFFVDPSYTSAVDPKTGYRQHFRLDDRINTKTFNTFIRDGFKDIEYKDSNLLFTFNWRQLASARNKIKKDVRAEKEAEISDKPWTITANIERLVYSKKADDGHGATVKVYPQVELLQLFESKNFDLRKNIKQQLLEHKPDAKFVGSFTYLFNIINKLRNSDAASDVDAIISPVYIDGFNSTKHKLGGYEWNGDANGAYNIARKGAILLDKLYEAKTLRDFKATVTRAEYDAYLTK

SEQ ID NO: 169 - protein, 1228 aa, synthetic construct
MWEDFTKLYSLSKTLRFELVPTESTEQQLKTSGVIQADKVRHESFKLAKKVLDEIHRKFITDSLVDLQLDHKQLQVFAEYSLNPASKSDAAKQAELIKLRRSIYMAFEQRFTATQAGWKEAYDCEKFISSPKSQLALIETFKPELADKLGMPEQDLEDLQTRFNKFASYFQGYNRNRENIYTTKGHAVEISHRTIDQNLLKFYKNVQKYEKYYTDKDILGGEAAPHFKVENYTSYITQDEINAYNEAIRSFRSNINQARQGLDKKEKQSLPMFEPLFKQILGDNTKKVLFEKLEGKEEYAELTRKLQQDMPSWVTALDEHANTHIMIPANPATVWFNAKAVNAVLHKHTTSPELIKIYLPKEFTSSRRAKDEETAKQNSFVSLRQLFAALDQAQVATELSLLKNVDRADMGASEAVWLAFCEDIQVKIKDARETVGAFSKIIAFENKSEKETLKKALDSFNDLYRMYTYFELRYKNEPVTSFDTDPDFYELFEDPDDQDSLFDADKDKRFSHYYDLIRNYLTKKSYSLDKWKLNFDCSQLLRGWDVNREPEKHGVILQNGENYHLAILNNESNKIFDRAKNKLLFQAEDNTAWQKMDYKLLPGPNKMLPKCLLPKSNRNKYGATDEVLDIYETGSFKKGDNFNLASLHKMIDFYKQAINRYEGWSMYEFTFRSTDQYQGIDQFYHDAESQGYRLGFVPINSVELQKMVDDGRIYLFRIQNKDLANPSSKTANMHTLYFRELFKSSSTIKLNGEAEIFFRPKTENLAASKTNRKTPQGTEVTELKRFRSDKIQLHLPVNLNYQTKSLINATFNQHTIERIKTNGVNAIIGIDRGEKHLAYVAIVDRSGKLLDEPISLNNITSKLPGGGKKVTPYFDLLDHREKERKENRQNWEAVRQIKDLKSGYIGHCIKQIVDLMIKHNAVVALENLNVGFKQGRSAIEKSVYNQLEQALLHKLQYVVDKSKPPQELFGAQNGVQLAPPSVVPKDTYNHMGFVFFVDPSYTSAVDPKTGYRQHFRLDDRINTKTFNTFIRDGFKDIEYKDSNLLFTFNWRQLASARNKIKKDVRAEKEAEISDKPWTITANIERLVYSKKADDGHGATVKVYPQVELLQLFESKNFDLRKNIKQQLLEHKPDAKFVGSFTYLFNIINKLRNSDAASDVDAIISPVYIDGFNSTKHKLGGYEWNGDANGAYNIARKGAILLDKLYEAKTLRDFKATVTRAEYDAYLTK

SEQ ID NO: 170 - protein, 1228 aa, synthetic construct
MWEDFTKLYSLSKTLRFELVPTESTEQQLKTSGVIQADKVRHESFKLAKKVLDEIHRKFITDSLVDLQLDHKQLQVFAEYSLNPASKSDAAKQAELIKLRRSIYMAFEQRFTATQAGWKEAYDCEKFISSPKSQLALIETFKPELADKLGMPEQDLEDLQTRFNKFASYFQGYNRNRENIYTTKGHAVEISHRTIDQNLLKFYKNVQKYEKYYTDKDILGGEAAPHFKVENYTSYITQDEINAYNEAIRSFRSNIAQARQGLDKKEKQSLPMFEPLFKQILGDNTKKVLFEKLEGKEEYAELTRKLQQDMPSWVTALDEHANTHIMIPANPATVWFNAKAVNAVLHKHTTSPELIKIYLPKEFTSSRRAKDEETAKQNSFVSLRQLFAALDQAQVATELSLLKNVDRADMGASEAVWLAFCEDIQVKIKDARETVGAFSKIIAFENKSEKETLKKALDSFNDLYRMYTYFELRYKNEPVTSFDTDPDFYELFEDPDDQDSLFDADKDKRFSHYYDLIRNYLTKKSYSLDKWKLNFDCSQLLTGWDVNREPEKHGVILQNGENYHLAILNNESNKIFDRAKNKLLFQAEDNTAWQKMDYKLLPGPNKMLPKCLLPKSNRNKYGATDEVLDIYETGSFKKGDNFNLASLHKMIDFYKQAINRYEGWSMYEFTFRSTDQYQGIDQFYHDAESQGYRLGFVPINSVELQKMVDDGRIYLFRIQNKDLANPSSKTANMHTLYFRELFKSSSTIKLNGEAEIFFRPKTENLAASKTNRKTPQGTEVTELKRFRSDKIQLHLPVNLNYQTKSLINATFNQHTIERIKTNGVNAIIGIDRGEKHLAYVAIVDRSGKLLDEPISLNNITSKLPGGGKKVTPYFDLLDHREKERKENRQNWEAVRQIKDLKSGYIGHCIKQIVDLMIKHNAVVALENLNVGFKQGRSAIEKSVYNQLEQALLHKLQYVVDKSKPPQELFGAQNGVQLAPPSVVPKDTYNHMGFVFFVDPSYTSAVDPKTGYRQHFRLDDRINTKTFNTFIRDGFKDIEYKDSNLLFTFNWRQLASARNKIKKDVRAEKEAEISDKPWTITANIERLVYSKKADDGHGATVKVYPQVELLQLFESKNFDLRKNIKQQLLEHKPDAKFVGSFTYLFNIINKLRNSDAASDVDAIISPVYIDGFNSTKHKLGGYEWNGDANGAYNIARKGAILLDKLYEAKTLRDFKATVTRAEYDAYLTK

SEQ ID NO: 171 - protein, 1228 aa, synthetic construct
MWEDFTKLYSLSKTLRFELVPTESTEQQLKTSGVIQADKVRHESFKLAKKVLDEIHRKFITDSLVDLQLDHKQLQVFAEYSLNPASKSDAAKQAELIKLRRSIYMAFEQRFTATQAGWKEAYDCEKFISSPKSQLALIETFKPELADKLGMPEQDLEDLQTRFNKFASYFQGYNKNRENIYTTKGHAVEISHRTIDQNLLKFYKNVQKYEKYYTDKDILGGEAAPHFKVENYTSYITQDEINAYNEAIRSFRSNINQARQGLDKKEKQSLPMFEPLFKQILGDNTKKVLFEKLEGKEEYAELTRKLQQDMPSWVTALDEHANTHIMIPANPATVWFNAKAVNAVLHKHTTSPELIKIYLPKEFTSSRRAKDEETAKQNSFVSLRQLFAALDQAQVATELSLLKNVDRADMGASEAVWLAFCEDIQVKIKDARETVGAFSKIIAFENKSEKETLKKALDSFNDLYRMYTYFELRYKNEPVTSFDTDPDFYELFEDPDDQDSLFDADKDKRFSHYYDLIRNYLTKKSYSLDKWKLNFDRSQLLTGWDVNKEPEKHGVILQNGENYHLAILNNESNKIFDRAKNKLLFQAEDNTAWQKMDYKLLPGPNKMLPKCLLPKSNRNKYGATDEVLDIYETGSFKKGDNFNLASLHKMIDFYKQAINRYEGWSMYEFTFRSTDQYQGIDQFYHDAESQGYRLGFVPINSVELQKMVDDGRIYLFRIQNKDLANPSSKTANMHTLYFRELFKSSSTIKLNGEAEIFFRPKTENLAASKTNRKTPQGTEVTELKRFRSDKIQLHLPVNLNYQTKSLINATFNQHTIERIKTNGVNAIIGIDRGEKHLAYVAIVDRSGKLLDEPISLNNITSKLPGGGKKVTPYFDLLDHREKERKENRQNWEAVRQIKDLKSGYIGHCIKQIVDLMIKHNAVVALENLNVGFKQGRSAIEKSVYNQLEQALLHKLQYVVDKSKPPQELFGAQNGVQLAPPSVVPKDTYNHMGFVFFVDPSYTSAVDPKTGYRQHFRLDDRINTKTFNTFIRDGFKDIEYKDSNLLFTFNWRQLASARNKIKKDVRAEKEAEISDKPWTITANIERLVYSKKADDGHGATVKVYPQVELLQLFESKNFDLRKNIKQQLLEHKPDAKFVGSFTYLFNIINKLRNSDAASDVDAIISPVYIDGFNSTKHKLGGYEWNGDANGAYNIARKGAILLDKLYEAKTLRDFKATVTRAEYDAYLTK

SEQ ID NO: 172 - protein, 1233 aa, synthetic construct
MKDLSTFISQYDVSKTLRFELIPTPETEKHIREKGILDADEKLAVSYKRMKETMDEFHKDFIDRALSTAQLRHIDEYCALYWADAEEKNSKKYADRLKAASAELRKEIVQCFDADDAFKKIDKKEFIKDELETWINEKHPMLYYDPDFKKFTTYFEGYRRNRMNLYSDEEKVGSVAYRLIGENLPKFLDNIRIYEKVKASKVAGKFSKLCKDMEQYMNGRSLDEMFSARAYPSTLTQTQIDQYNTVIGGRNDGSSKLQGLNEYINLYNQQHPDAKLPIFKMLYKQLLSDRISASWLPEAFDSAQALFDGVEAFAQEVLTDTVLNDLQTVLNELPERDAALIYVRKSSVNDISSRLFGYYSVFNDALDAMGENEKTGYISLARLQSAVDGYIPTLDKDEYESIVSEYAPNCVTACFFDNLHEYIENISDAYEALRPCFGVEHDENYQLSEKERTWLKAYLDGVIALLHFIKPLYIDKDFALEKDEVFYSDFMPLYEKLSQVTKLYDKVRSFASKKPYSTKKIKLNFGCSTLLSGWDVNKETQNLGVLLIKDGNYYLGIMDKANNKLFSDAPECQSEDCYQKVVYKLLQEPYKMLPKVFFSKSRINEFAPSEEIISIRINETFKKGPKFIAADCHKLVDFYKTSISKHPDWRRFEFKFSPTGTYVDINDFYQEVAEQGYKLFFKPVSTAYIDSAVSEGKLYLFRIYNKDFSPSAKGKPNLHTMYWKALFDEKNLADVVYKLSGHGEVFYRSRSIDDEKRVIHPANKPIDKRNSGMSGETSVFDYDIIKDRRYTVDKFQFHVPISLNFKAHGVSNFNEKVCTYLKNNPDVNIIGIDRGERNLLYISMIDRSGRVVKDSRGNYIQYSLNTITGEYKNADGAPVKFSTPYHELLDKKEKNRREARENWGDIESIKELKAGYMSQVVYHISRLMVEYNAIVVMEDLNSGFKNGRKKVEKQVYQNFEKALIEKLNYLVFKDYPVDTCGGLYRALQLTDKFKSFGMLTKQTGFLFYVPAWNTSKIDPVTGFVDMLKPKYKSIPDARVFFSKFNYIRYNDQKDWFEFSFDYGNFTDRADGTKTDWTLCTYGSLRYAYNKSLNNGHGGYEKWNVTDKLKALFSANGIEYADGKDLKTKITAQNKADFYAILVKCLQVTLAMRYSSVEDGKDFILSPVADENGVFFCSEGRNDGLPQDADANGAYNIARKGLCVLEHIDVAEKYSDWSTKISNKEWLNFVQTRA

SEQ ID NO: 173 - protein, 1233 aa, synthetic construct
MKDLSTFISQYDVSKTLRFELIPTPETEKHIREKGILDADEKLAVSYKRMKETMDEFHKDFIDRALSTAQLRHIDEYCALYWADAEEKNSKKYADRLKAASAELRKEIVQCFDADDAFKKIDKKEFIKDELETWINEKHPMLYYDPDFKKFTTYFEGYRKNRMNLYSDEEKVGSVAYRLIGENLPKFLDNIRIYEKVKASKVAGKFSKLCKDMEQYMNGRSLDEMFSARAYPSTLTQTQIDQYNTVIGGRNDGSSKLQGLNEYINLYNQQHPDAKLPIFKMLYKQLLSDRISASWLPEAFDSAQALFDGVEAFAQEVLTDTVLNDLQTVLNELPERDAALIYVRKSSVNDISSRLFGYYSVFNDALDAMGENEKTGYISLARLQSAVDGYIPTLDKDEYESIVSEYAPNCVTACFFDNLHEYIENISDAYEALRPCFGVEHDENYQLSEKERTWLKAYLDGVIALLHFIKPLYIDKDFALEKDEVFYSDFMPLYEKLSQVTKLYDKVRSFASKKPYSTKKIKLNFGCSTLLRGWDVNKETQNLGVLLIKDGNYYLGIMDKANNKLFSDAPECQSEDCYQKVVYKLLQEPYKMLPKVFFSKSRINEFAPSEEIISIRINETFKKGPKFIAADCHKLVDFYKTSISKHPDWRRFEFKFSPTGTYVDINDFYQEVAEQGYKLFFKPVSTAYIDSAVSEGKLYLFRIYNKDFSPSAKGKPNLHTMYWKALFDEKNLADVVYKLSGHGEVFYRSRSIDDEKRVIHPANKPIDKRNSGMSGETSVFDYDIIKDRRYTVDKFQFHVPISLNFKAHGVSNFNEKVCTYLKNNPDVNIIGIDRGERNLLYISMIDRSGRVVKDSRGNYIQYSLNTITGEYKNADGAPVKFSTPYHELLDKKEKNRREARENWGDIESIKELKAGYMSQVVYHISRLMVEYNAIVVMEDLNSGFKNGRKKVEKQVYQNFEKALIEKLNYLVFKDYPVDTCGGLYRALQLTDKFKSFGMLTKQTGFLFYVPAWNTSKIDPVTGFVDMLKPKYKSIPDARVFFSKFNYIRYNDQKDWFEFSFDYGNFTDRADGTKTDWTLCTYGSLRYAYNKSLNNGHGGYEKWNVTDKLKALFSANGIEYADGKDLKTKITAQNKADFYAILVKCLQVTLAMRYSSVEDGKDFILSPVADENGVFFCSEGRNDGLPQDADANGAYNIARKGLCVLEHIDVAEKYSDWSTKISNKEWLNFVQTRA

SEQ ID NO: 174 - protein, 1233 aa, synthetic construct
MKDLSTFISQYDVSKTLRFELIPTPETEKHIREKGILDADEKLAVSYKRMKETMDEFHKDFIDRALSTAQLRHIDEYCALYWADAEEKNSKKYADRLKAASAELRKEIVQCFDADDAFKKIDKKEFIKDELETWINEKHPMLYYDPDFKKFTTYFEGYRKNRMNLYSDEEKVGSVAYRLIGENLPKFLDNIRIYEKVKASKVAGKFSKLCKDMEQYMNGRSLDEMFSARAYPSTLTQTQIDQYNTVIGGRNDGSSKLQGLNEYINLYNQQHPDAKLPIFKMLYKQLLSDRISASWLPEAFDSAQALFDGVEAFAQEVLTDTVLNDLQTVLNELPERDAALIYVRKSSVNDISSRLFGYYSVFNDALDAMGENEKTGYISLARLQSAVDGYIPTLDKDEYESIVSEYAPNCVTACFFDNLHEYIENISDAYEALRPCFGVEHDENYQLSEKERTWLKAYLDGVIALLHFIKPLYIDKDFALEKDEVFYSDFMPLYEKLSQVTKLYDKVRSFASKKPYSTKKIKLNFGCSTLLSGWDVNRETQNLGVLLIKDGNYYLGIMDKANNKLFSDAPECQSEDCYQKVVYKLLQEPYKMLPKVFFSKSRINEFAPSEEIISIRINETFKKGPKFIAADCHKLVDFYKTSISKHPDWRRFEFKFSPTGTYVDINDFYQEVAEQGYKLFFKPVSTAYIDSAVSEGKLYLFRIYNKDFSPSAKGKPNLHTMYWKALFDEKNLADVVYKLSGHGEVFYRSRSIDDEKRVIHPANKPIDKRNSGMSGETSVFDYDIIKDRRYTVDKFQFHVPISLNFKAHGVSNFNEKVCTYLKNNPDVNIIGIDRGERNLLYISMIDRSGRVVKDSRGNYIQYSLNTITGEYKNADGAPVKFSTPYHELLDKKEKNRREARENWGDIESIKELKAGYMSQVVYHISRLMVEYNAIVVMEDLNSGFKNGRKKVEKQVYQNFEKALIEKLNYLVFKDYPVDTCGGLYRALQLTDKFKSFGMLTKQTGFLFYVPAWNTSKIDPVTGFVDMLKPKYKSIPDARVFFSKFNYIRYNDQKDWFEFSFDYGNFTDRADGTKTDWTLCTYGSLRYAYNKSLNNGHGGYEKWNVTDKLKALFSANGIEYADGKDLKTKITAQNKADFYAILVKCLQVTLAMRYSSVEDGKDFILSPVADENGVFFCSEGRNDGLPQDADANGAYNIARKGLCVLEHIDVAEKYSDWSTKISNKEWLNFVQTRA

SEQ ID NO: 175 - protein, 1233 aa, synthetic construct
MKDLSTFISQYDVSKTLRFELIPTPETEKHIREKGILDADEKLAVSYKRMKETMDEFHKDFIDRALSTAQLRHIDEYCALYWADAEEKNSKKYADRLKAASAELRKEIVQCFDADDAFKKIDKKEFIKDELETWINEKHPMLYYDPDFKKFTTYFEGYRRNRMNLYSDEEKVGSVAYRLIGENLPKFLDNIRIYEKVKASKVAGKFSKLCKDMEQYMNGRSLDEMFSARAYPSTLTQTQIDQYNTVIGGRNDGSSKLQGLNEYINLYNQQHPDAKLPIFKMLYKQLLSDRISASWLPEAFDSAQALFDGVEAFAQEVLTDTVLNDLQTVLNELPERDAALIYVRKSSVNDISSRLFGYYSVFNDALDAMGENEKTGYISLARLQSAVDGYIPTLDKDEYESIVSEYAPNCVTACFFDNLHEYIENISDAYEALRPCFGVEHDENYQLSEKERTWLKAYLDGVIALLHFIKPLYIDKDFALEKDEVFYSDFMPLYEKLSQVTKLYDKVRSFASKKPYSTKKIKLNFGCSTLLRGWDVNKETQNLGVLLIKDGNYYLGIMDKANNKLFSDAPECQSEDCYQKVVYKLLQEPYKMLPKVFFSKSRINEFAPSEEIISIRINETFKKGPKFIAADCHKLVDFYKTSISKHPDWRRFEFKFSPTGTYVDINDFYQEVAEQGYKLFFKPVSTAYIDSAVSEGKLYLFRIYNKDFSPSAKGKPNLHTMYWKALFDEKNLADVVYKLSGHGEVFYRSRSIDDEKRVIHPANKPIDKRNSGMSGETSVFDYDIIKDRRYTVDKFQFHVPISLNFKAHGVSNFNEKVCTYLKNNPDVNIIGIDRGERNLLYISMIDRSGRVVKDSRGNYIQYSLNTITGEYKNADGAPVKFSTPYHELLDKKEKNRREARENWGDIESIKELKAGYMSQVVYHISRLMVEYNAIVVMEDLNSGFKNGRKKVEKQVYQNFEKALIEKLNYLVFKDYPVDTCGGLYRALQLTDKFKSFGMLTKQTGFLFYVPAWNTSKIDPVTGFVDMLKPKYKSIPDARVFFSKFNYIRYNDQKDWFEFSFDYGNFTDRADGTKTDWTLCTYGSLRYAYNKSLNNGHGGYEKWNVTDKLKALFSANGIEYADGKDLKTKITAQNKADFYAILVKCLQVTLAMRYSSVEDGKDFILSPVADENGVFFCSEGRNDGLPQDADANGAYNIARKGLCVLEHIDVAEKYSDWSTKISNKEWLNFVQTRA

SEQ ID NO: 176 - protein, 1233 aa, synthetic construct
MKDLSTFISQYDVSKTLRFELIPTPETEKHIREKGILDADEKLAVSYKRMKETMDEFHKDFIDRALSTAQLRHIDEYCALYWADAEEKNSKKYADRLKAASAELRKEIVQCFDADDAFKKIDKKEFIKDELETWINEKHPMLYYDPDFKKFTTYFEGYRRNRMNLYSDEEKVGSVAYRLIGENLPKFLDNIRIYEKVKASKVAGKFSKLCKDMEQYMNGRSLDEMFSARAYPSTLTQTQIDQYNTVIGGRNDGSSKLQGLNEYINLYNQQHPDAKLPIFKMLYKQLLSDRISASWLPEAFDSAQALFDGVEAFAQEVLTDTVLNDLQTVLNELPERDAALIYVRKSSVNDISSRLFGYYSVFNDALDAMGENEKTGYISLARLQSAVDGYIPTLDKDEYESIVSEYAPNCVTACFFDNLHEYIENISDAYEALRPCFGVEHDENYQLSEKERTWLKAYLDGVIALLHFIKPLYIDKDFALEKDEVFYSDFMPLYEKLSQVTKLYDKVRSFASKKPYSTKKIKLNFGCSTLLSGWDVNRETQNLGVLLIKDGNYYLGIMDKANNKLFSDAPECQSEDCYQKVVYKLLQEPYKMLPKVFFSKSRINEFAPSEEIISIRINETFKKGPKFIAADCHKLVDFYKTSISKHPDWRRFEFKFSPTGTYVDINDFYQEVAEQGYKLFFKPVSTAYIDSAVSEGKLYLFRIYNKDFSPSAKGKPNLHTMYWKALFDEKNLADVVYKLSGHGEVFYRSRSIDDEKRVIHPANKPIDKRNSGMSGETSVFDYDIIKDRRYTVDKFQFHVPISLNFKAHGVSNFNEKVCTYLKNNPDVNIIGIDRGERNLLYISMIDRSGRVVKDSRGNYIQYSLNTITGEYKNADGAPVKFSTPYHELLDKKEKNRREARENWGDIESIKELKAGYMSQVVYHISRLMVEYNAIVVMEDLNSGFKNGRKKVEKQVYQNFEKALIEKLNYLVFKDYPVDTCGGLYRALQLTDKFKSFGMLTKQTGFLFYVPAWNTSKIDPVTGFVDMLKPKYKSIPDARVFFSKFNYIRYNDQKDWFEFSFDYGNFTDRADGTKTDWTLCTYGSLRYAYNKSLNNGHGGYEKWNVTDKLKALFSANGIEYADGKDLKTKITAQNKADFYAILVKCLQVTLAMRYSSVEDGKDFILSPVADENGVFFCSEGRNDGLPQDADANGAYNIARKGLCVLEHIDVAEKYSDWSTKISNKEWLNFVQTRA

SEQ ID NO: 177 - protein, 1233 aa, synthetic construct
MKDLSTFISQYDVSKTLRFELIPTPETEKHIREKGILDADEKLAVSYKRMKETMDEFHKDFIDRALSTAQLRHIDEYCALYWADAEEKNSKKYADRLKAASAELRKEIVQCFDADDAFKKIDKKEFIKDELETWINEKHPMLYYDPDFKKFTTYFEGYRKNRMNLYSDEEKVGSVAYRLIGENLPKFLDNIRIYEKVKASKVAGKFSKLCKDMEQYMNGRSLDEMFSARAYPSTLTQTQIDQYNTVIGGRNDGSSKLQGLNEYINLYNQQHPDAKLPIFKMLYKQLLSDRISASWLPEAFDSAQALFDGVEAFAQEVLTDTVLNDLQTVLNELPERDAALIYVRKSSVNDISSRLFGYYSVFNDALDAMGENEKTGYISLARLQSAVDGYIPTLDKDEYESIVSEYAPNCVTACFFDNLHEYIENISDAYEALRPCFGVEHDENYQLSEKERTWLKAYLDGVIALLHFIKPLYIDKDFALEKDEVFYSDFMPLYEKLSQVTKLYDKVRSFASKKPYSTKKIKLNFGCSTLLRGWDVNRETQNLGVLLIKDGNYYLGIMDKANNKLFSDAPECQSEDCYQKVVYKLLQEPYKMLPKVFFSKSRINEFAPSEEIISIRINETFKKGPKFIAADCHKLVDFYKTSISKHPDWRRFEFKFSPTGTYVDINDFYQEVAEQGYKLFFKPVSTAYIDSAVSEGKLYLFRIYNKDFSPSAKGKPNLHTMYWKALFDEKNLADVVYKLSGHGEVFYRSRSIDDEKRVIHPANKPIDKRNSGMSGETSVFDYDIIKDRRYTVDKFQFHVPISLNFKAHGVSNFNEKVCTYLKNNPDVNIIGIDRGERNLLYISMIDRSGRVVKDSRGNYIQYSLNTITGEYKNADGAPVKFSTPYHELLDKKEKNRREARENWGDIESIKELKAGYMSQVVYHISRLMVEYNAIVVMEDLNSGFKNGRKKVEKQVYQNFEKALIEKLNYLVFKDYPVDTCGGLYRALQLTDKFKSFGMLTKQTGFLFYVPAWNTSKIDPVTGFVDMLKPKYKSIPDARVFFSKFNYIRYNDQKDWFEFSFDYGNFTDRADGTKTDWTLCTYGSLRYAYNKSLNNGHGGYEKWNVTDKLKALFSANGIEYADGKDLKTKITAQNKADFYAILVKCLQVTLAMRYSSVEDGKDFILSPVADENGVFFCSEGRNDGLPQDADANGAYNIARKGLCVLEHIDVAEKYSDWSTKISNKEWLNFVQTRA

SEQ ID NO: 178 - protein, 1233 aa, synthetic construct
MKDLSTFISQYDVSKTLRFELIPTPETEKHIREKGILDADEKLAVSYKRMKETMDEFHKDFIDRALSTAQLRHIDEYCALYWADAEEKNSKKYADRLKAASAELRKEIVQCFDADDAFKKIDKKEFIKDELETWINEKHPMLYYDPDFKKFTTYFEGYRRNRMNLYSDEEKVGSVAYRLIGENLPKFLDNIRIYEKVKASKVAGKFSKLCKDMEQYMNGRSLDEMFSARAYPSTLTQTQIDQYNTVIGGRNDGSSKLQGLNEYINLYNQQHPDAKLPIFKMLYKQLLSDRISASWLPEAFDSAQALFDGVEAFAQEVLTDTVLNDLQTVLNELPERDAALIYVRKSSVNDISSRLFGYYSVFNDALDAMGENEKTGYISLARLQSAVDGYIPTLDKDEYESIVSEYAPNCVTACFFDNLHEYIENISDAYEALRPCFGVEHDENYQLSEKERTWLKAYLDGVIALLHFIKPLYIDKDFALEKDEVFYSDFMPLYEKLSQVTKLYDKVRSFASKKPYSTKKIKLNFGCSTLLRGWDVNRETQNLGVLLIKDGNYYLGIMDKANNKLFSDAPECQSEDCYQKVVYKLLQEPYKMLPKVFFSKSRINEFAPSEEIISIRINETFKKGPKFIAADCHKLVDFYKTSISKHPDWRRFEFKFSPTGTYVDINDFYQEVAEQGYKLFFKPVSTAYIDSAVSEGKLYLFRIYNKDFSPSAKGKPNLHTMYWKALFDEKNLADVVYKLSGHGEVFYRSRSIDDEKRVIHPANKPIDKRNSGMSGETSVFDYDIIKDRRYTVDKFQFHVPISLNFKAHGVSNFNEKVCTYLKNNPDVNIIGIDRGERNLLYISMIDRSGRVVKDSRGNYIQYSLNTITGEYKNADGAPVKFSTPYHELLDKKEKNRREARENWGDIESIKELKAGYMSQVVYHISRLMVEYNAIVVMEDLNSGFKNGRKKVEKQVYQNFEKALIEKLNYLVFKDYPVDTCGGLYRALQLTDKFKSFGMLTKQTGFLFYVPAWNTSKIDPVTGFVDMLKPKYKSIPDARVFFSKFNYIRYNDQKDWFEFSFDYGNFTDRADGTKTDWTLCTYGSLRYAYNKSLNNGHGGYEKWNVTDKLKALFSANGIEYADGKDLKTKITAQNKADFYAILVKCLQVTLAMRYSSVEDGKDFILSPVADENGVFFCSEGRNDGLPQDADANGAYNIARKGLCVLEHIDVAEKYSDWSTKISNKEWLNFVQTRA

SEQ ID NO: 179 - protein, 1233 aa, synthetic construct
MKDLSTFISQYDVSKTLRFELIPTPETEKHIREKGILDADEKLAVSYKRMKETMDEFHKDFIDRALSTAQLRHIDEYCALYWADAEEKNSKKYADRLKAASAELRKEIVQCFDADDAFKKIDKKEFIKDELETWINEKHPMLYYDPDFKKFTTYFEGYRRNRMNLYSDEEKVGSVAYRLIGENLPKFLDNIRIYEKVKASKVAGKFSKLCKDMEQYMNGRSLDEMFSARAYPSTLTQTQIDQYNTVIGGRNDGSSKLQGLNEYIALYNQQHPDAKLPIFKMLYKQLLSDRISASWLPEAFDSAQALFDGVEAFAQEVLTDTVLNDLQTVLNELPERDAALIYVRKSSVNDISSRLFGYYSVFNDALDAMGENEKTGYISLARLQSAVDGYIPTLDKDEYESIVSEYAPNCVTACFFDNLHEYIENISDAYEALRPCFGVEHDENYQLSEKERTWLKAYLDGVIALLHFIKPLYIDKDFALEKDEVFYSDFMPLYEKLSQVTKLYDKVRSFASKKPYSTKKIKLNFGCSTLLSGWDVNRETQNLGVLLIKDGNYYLGIMDKANNKLFSDAPECQSEDCYQKVVYKLLQEPYKMLPKVFFSKSRINEFAPSEEIISIRINETFKKGPKFIAADCHKLVDFYKTSISKHPDWRRFEFKFSPTGTYVDINDFYQEVAEQGYKLFFKPVSTAYIDSAVSEGKLYLFRIYNKDFSPSAKGKPNLHTMYWKALFDEKNLADVVYKLSGHGEVFYRSRSIDDEKRVIHPANKPIDKRNSGMSGETSVFDYDIIKDRRYTVDKFQFHVPISLNFKAHGVSNFNEKVCTYLKNNPDVNIIGIDRGERNLLYISMIDRSGRVVKDSRGNYIQYSLNTITGEYKNADGAPVKFSTPYHELLDKKEKNRREARENWGDIESIKELKAGYMSQVVYHISRLMVEYNAIVVMEDLNSGFKNGRKKVEKQVYQNFEKALIEKLNYLVFKDYPVDTCGGLYRALQLTDKFKSFGMLTKQTGFLFYVPAWNTSKIDPVTGFVDMLKPKYKSIPDARVFFSKFNYIRYNDQKDWFEFSFDYGNFTDRADGTKTDWTLCTYGSLRYAYNKSLNNGHGGYEKWNVTDKLKALFSANGIEYADGKDLKTKITAQNKADFYAILVKCLQVTLAMRYSSVEDGKDFILSPVADENGVFFCSEGRNDGLPQDADANGAYNIARKGLCVLEHIDVAEKYSDWSTKISNKEWLNFVQTRA

SEQ ID NO: 180 - protein, 1233 aa, synthetic construct
MKDLSTFISQYDVSKTLRFELIPTPETEKHIREKGILDADEKLAVSYKRMKETMDEFHKDFIDRALSTAQLRHIDEYCALYWADAEEKNSKKYADRLKAASAELRKEIVQCFDADDAFKKIDKKEFIKDELETWINEKHPMLYYDPDFKKFTTYFEGYRKNRMNLYSDEEKVGSVAYRLIGENLPKFLDNIRIYEKVKASKVAGKFSKLCKDMEQYMNGRSLDEMFSARAYPSTLTQTQIDQYNTVIGGRNDGSSKLQGLNEYINLYNQQHPDAKLPIFKMLYKQLLSDRISASWLPEAFDSAQALFDGVEAFAQEVLTDTVLNDLQTVLNELPERDAALIYVRKSSVNDISSRLFGYYSVFNDALDAMGENEKTGYISLARLQSAVDGYIPTLDKDEYESIVSEYAPNCVTACFFDNLHEYIENISDAYEALRPCFGVEHDENYQLSEKERTWLKAYLDGVIALLHFIKPLYIDKDFALEKDEVFYSDFMPLYEKLSQVTKLYDKVRSFASKKPYSTKKIKLNFGRSTLLSGWDVNKETQNLGVLLIKDGNYYLGIMDKANNKLFSDAPECQSEDCYQKVVYKLLQEPYKMLPKVFFSKSRINEFAPSEEIISIRINETFKKGPKFIAADCHKLVDFYKTSISKHPDWRRFEFKFSPTGTYVDINDFYQEVAEQGYKLFFKPVSTAYIDSAVSEGKLYLFRIYNKDFSPSAKGKPNLHTMYWKALFDEKNLADVVYKLSGHGEVFYRSRSIDDEKRVIHPANKPIDKRNSGMSGETSVFDYDIIKDRRYTVDKFQFHVPISLNFKAHGVSNFNEKVCTYLKNNPDVNIIGIDRGERNLLYISMIDRSGRVVKDSRGNYIQYSLNTITGEYKNADGAPVKFSTPYHELLDKKEKNRREARENWGDIESIKELKAGYMSQVVYHISRLMVEYNAIVVMEDLNSGFKNGRKKVEKQVYQNFEKALIEKLNYLVFKDYPVDTCGGLYRALQLTDKFKSFGMLTKQTGFLFYVPAWNTSKIDPVTGFVDMLKPKYKSIPDARVFFSKFNYIRYNDQKDWFEFSFDYGNFTDRADGTKTDWTLCTYGSLRYAYNKSLNNGHGGYEKWNVTDKLKALFSANGIEYADGKDLKTKITAQNKADFYAILVKCLQVTLAMRYSSVEDGKDFILSPVADENGVFFCSEGRNDGLPQDADANGAYNIARKGLCVLEHIDVAEKYSDWSTKISNKEWLNFVQTRA

SEQ ID NO: 181 - protein, 1197 aa, synthetic construct
MLSNFTNQYQLSKTLRFELKPVGDTLKHIEKSGLIAQDEIRSQEYQEVKTIIDKYHKAFIDEALQNVVLSNLEEYEALFFERNRDEKAFEKLQAVLRKEIVAHFKQHPQYKTLFKKELIKADLKNWQELSDAEKELVSHFDNFTTYFTGFHRNRANMYTDEAKHSSIAYRIIHENLPIFLTNKKLFETIKQKAPHLAQETQDALLEYLSGAIVEDMFELSYFNHLLSQTHIDLYNQMIGGVKQDSLKIQGLNEKINLYRQANGLSKRELPNLKPLHKQILSDRETLSWLPESFESDEELMQGVQAYFESEVLAFECCDGKVNLLEKLPELLHQTQDYDFSKVYFKNDLALTAASQAIFKDYRIIKEALWEVNKPKKSKDLVADEEKFFNKKNSYFSIEQIDGALNSAQLSANMMHYFQSESTKVIEQIQLTYNDWKRNSSNKELLKAFLDALLSYQRLLKPLNAPNDLEKDVAFYAYFDAYFTSLCGVVKLYDKVRNFMTKKPYSLEKFKLNFENSTLLDGWDVNKESDNTAILFRKEGLYYLGIMNKKYNKVFRNISSSQDEGYQKIDYKLLPGANKMLPKVFFSDKNKEYFKPNAKLLERYKAGEHKKGDNFDLDFCHELIDFFKTSIEKHQDWKHFAYQFSPTESYEDLSGFYREVEQQGYKISYKNIAASFIDTLVAEGKLYFFQIYNKDFSPYSKGTPNMHTLYWRALFDEKNLADVIYKLNGQAEIFFRKKSIEYSQEKLQKGHHHEMLKDKFAYPIIKDRRFAFDKFQFHVPITLNFKAEGNENITPKTFEYIRSNPDNIKVIGIDRGERHLLYLSLIDAEGKIVEQFTLNQIINSYNGKDHVIDYHAKLDAKEKDRDKARKEWGTVENIKELKEGYLSHVIHKIATLIIEHGAVVAMEDLNFGFKRGRFKVEKQVYQKFEKALIDKLNYLVDKKKEPHKLGGLLNALQLTSKFQSFEKMGKQNGFLFYVPAWNTSKIDPVTGFVNLFDTRYASVEKSKAFFTKFQSICYNEAKDYFELVFDYNDFTEKAKETRSEWTLCTYGERIVSFRNAEKNHQWDSKTIHLTTEFKNLFGELHGNDVKEYILEQNSVEFFKSLIYLLKITLQMRNSITGTDIDYLVSPVADEAGNFYDSRKADTSLPKDADANGAYNIARKGLMLMHRIQNAEDLKKVNLAISNRDWLRNAQGLDK

SEQ ID NO: 182 - protein, 1197 aa, synthetic construct
MLSNFTNQYQLSKTLRFELKPVGDTLKHIEKSGLIAQDEIRSQEYQEVKTIIDKYHKAFIDEALQNVVLSNLEEYEALFFERNRDEKAFEKLQAVLRKEIVAHFKQHPQYKTLFKKELIKADLKNWQELSDAEKELVSHFDNFTTYFTGFHENRANMYTDEAKHSSIAYRIIHENLPIFLTNKKLFETIKQKAPHLAQETQDALLEYLSGAIVEDMFELSYFNHLLSQTHIDLYNQMIGGVKQDSLKIQGLNEKINLYRQANGLSKRELPNLKPLHKQILSDRETLSWLPESFESDEELMQGVQAYFESEVLAFECCDGKVNLLEKLPELLHQTQDYDFSKVYFKNDLALTAASQAIFKDYRIIKEALWEVNKPKKSKDLVADEEKFFNKKNSYFSIEQIDGALNSAQLSANMMHYFQSESTKVIEQIQLTYNDWKRNSSNKELLKAFLDALLSYQRLLKPLNAPNDLEKDVAFYAYFDAYFTSLCGVVKLYDKVRNFMTKKPYSLEKFKLNFENSTLLRGWDVNKESDNTAILFRKEGLYYLGIMNKKYNKVFRNISSSQDEGYQKIDYKLLPGANKMLPKVFFSDKNKEYFKPNAKLLERYKAGEHKKGDNFDLDFCHELIDFFKTSIEKHQDWKHFAYQFSPTESYEDLSGFYREVEQQGYKISYKNIAASFIDTLVAEGKLYFFQIYNKDFSPYSKGTPNMHTLYWRALFDEKNLADVIYKLNGQAEIFFRKKSIEYSQEKLQKGHHHEMLKDKFAYPIIKDRRFAFDKFQFHVPITLNFKAEGNENITPKTFEYIRSNPDNIKVIGIDRGERHLLYLSLIDAEGKIVEQFTLNQIINSYNGKDHVIDYHAKLDAKEKDRDKARKEWGTVENIKELKEGYLSHVIHKIATLIIEHGAVVAMEDLNFGFKRGRFKVEKQVYQKFEKALIDKLNYLVDKKKEPHKLGGLLNALQLTSKFQSFEKMGKQNGFLFYVPAWNTSKIDPVTGFVNLFDTRYASVEKSKAFFTKFQSICYNEAKDYFELVFDYNDFTEKAKETRSEWTLCTYGERIVSFRNAEKNHQWDSKTIHLTTEFKNLFGELHGNDVKEYILEQNSVEFFKSLIYLLKITLQMRNSITGTDIDYLVSPVADEAGNFYDSRKADTSLPKDADANGAYNIARKGLMLMHRIQNAEDLKKVNLAISNRDWLRNAQGLDK

SEQ ID NO: 183 - protein, 1197 aa, synthetic construct
MLSNFTNQYQLSKTLRFELKPVGDTLKHIEKSGLIAQDEIRSQEYQEVKTIIDKYHKAFIDEALQNVVLSNLEEYEALFFERNRDEKAFEKLQAVLRKEIVAHFKQHPQYKTLFKKELIKADLKNWQELSDAEKELVSHFDNFTTYFTGFHENRANMYTDEAKHSSIAYRIIHENLPIFLTNKKLFETIKQKAPHLAQETQDALLEYLSGAIVEDMFELSYFNHLLSQTHIDLYNQMIGGVKQDSLKIQGLNEKINLYRQANGLSKRELPNLKPLHKQILSDRETLSWLPESFESDEELMQGVQAYFESEVLAFECCDGKVNLLEKLPELLHQTQDYDFSKVYFKNDLALTAASQAIFKDYRIIKEALWEVNKPKKSKDLVADEEKFFNKKNSYFSIEQIDGALNSAQLSANMMHYFQSESTKVIEQIQLTYNDWKRNSSNKELLKAFLDALLSYQRLLKPLNAPNDLEKDVAFYAYFDAYFTSLCGVVKLYDKVRNFMTKKPYSLEKFKLNFENSTLLDGWDVNRESDNTAILFRKEGLYYLGIMNKKYNKVFRNISSSQDEGYQKIDYKLLPGANKMLPKVFFSDKNKEYFKPNAKLLERYKAGEHKKGDNFDLDFCHELIDFFKTSIEKHQDWKHFAYQFSPTESYEDLSGFYREVEQQGYKISYKNIAASFIDTLVAEGKLYFFQIYNKDFSPYSKGTPNMHTLYWRALFDEKNLADVIYKLNGQAEIFFRKKSIEYSQEKLQKGHHHEMLKDKFAYPIIKDRRFAFDKFQFHVPITLNFKAEGNENITPKTFEYIRSNPDNIKVIGIDRGERHLLYLSLIDAEGKIVEQFTLNQIINSYNGKDHVIDYHAKLDAKEKDRDKARKEWGTVENIKELKEGYLSHVIHKIATLIIEHGAVVAMEDLNFGFKRGRFKVEKQVYQKFEKALIDKLNYLVDKKKEPHKLGGLLNALQLTSKFQSFEKMGKQNGFLFYVPAWNTSKIDPVTGFVNLFDTRYASVEKSKAFFTKFQSICYNEAKDYFELVFDYNDFTEKAKETRSEWTLCTYGERIVSFRNAEKNHQWDSKTIHLTTEFKNLFGELHGNDVKEYILEQNSVEFFKSLIYLLKITLQMRNSITGTDIDYLVSPVADEAGNFYDSRKADTSLPKDADANGAYNIARKGLMLMHRIQNAEDLKKVNLAISNRDWLRNAQGLDK

SEQ ID NO: 184 - protein, 1197 aa, synthetic construct
MLSNFTNQYQLSKTLRFELKPVGDTLKHIEKSGLIAQDEIRSQEYQEVKTIIDKYHKAFIDEALQNVVLSNLEEYEALFFERNRDEKAFEKLQAVLRKEIVAHFKQHPQYKTLFKKELIKADLKNWQELSDAEKELVSHFDNFTTYFTGFHRNRANMYTDEAKHSSIAYRIIHENLPIFLTNKKLFETIKQKAPHLAQETQDALLEYLSGAIVEDMFELSYFNHLLSQTHIDLYNQMIGGVKQDSLKIQGLNEKINLYRQANGLSKRELPNLKPLHKQILSDRETLSWLPESFESDEELMQGVQAYFESEVLAFECCDGKVNLLEKLPELLHQTQDYDFSKVYFKNDLALTAASQAIFKDYRIIKEALWEVNKPKKSKDLVADEEKFFNKKNSYFSIEQIDGALNSAQLSANMMHYFQSESTKVIEQIQLTYNDWKRNSSNKELLKAFLDALLSYQRLLKPLNAPNDLEKDVAFYAYFDAYFTSLCGVVKLYDKVRNFMTKKPYSLEKFKLNFENSTLLRGWDVNKESDNTAILFRKEGLYYLGIMNKKYNKVFRNISSSQDEGYQKIDYKLLPGANKMLPKVFFSDKNKEYFKPNAKLLERYKAGEHKKGDNFDLDFCHELIDFFKTSIEKHQDWKHFAYQFSPTESYEDLSGFYREVEQQGYKISYKNIAASFIDTLVAEGKLYFFQIYNKDFSPYSKGTPNMHTLYWRALFDEKNLADVIYKLNGQAEIFFRKKSIEYSQEKLQKGHHHEMLKDKFAYPIIKDRRFAFDKFQFHVPITLNFKAEGNENITPKTFEYIRSNPDNIKVIGIDRGERHLLYLSLIDAEGKIVEQFTLNQIINSYNGKDHVIDYHAKLDAKEKDRDKARKEWGTVENIKELKEGYLSHVIHKIATLIIEHGAVVAMEDLNFGFKRGRFKVEKQVYQKFEKALIDKLNYLVDKKKEPHKLGGLLNALQLTSKFQSFEKMGKQNGFLFYVPAWNTSKIDPVTGFVNLFDTRYASVEKSKAFFTKFQSICYNEAKDYFELVFDYNDFTEKAKETRSEWTLCTYGERIVSFRNAEKNHQWDSKTIHLTTEFKNLFGELHGNDVKEYILEQNSVEFFKSLIYLLKITLQMRNSITGTDIDYLVSPVADEAGNFYDSRKADTSLPKDADANGAYNIARKGLMLMHRIQNAEDLKKVNLAISNRDWLRNAQGLDK

SEQ ID NO: 185 - protein, 1197 aa, synthetic construct
MLSNFTNQYQLSKTLRFELKPVGDTLKHIEKSGLIAQDEIRSQEYQEVKTIIDKYHKAFIDEALQNVVLSNLEEYEALFFERNRDEKAFEKLQAVLRKEIVAHFKQHPQYKTLFKKELIKADLKNWQELSDAEKELVSHFDNFTTYFTGFHRNRANMYTDEAKHSSIAYRIIHENLPIFLTNKKLFETIKQKAPHLAQETQDALLEYLSGAIVEDMFELSYFNHLLSQTHIDLYNQMIGGVKQDSLKIQGLNEKINLYRQANGLSKRELPNLKPLHKQILSDRETLSWLPESFESDEELMQGVQAYFESEVLAFECCDGKVNLLEKLPELLHQTQDYDFSKVYFKNDLALTAASQAIFKDYRIIKEALWEVNKPKKSKDLVADEEKFFNKKNSYFSIEQIDGALNSAQLSANMMHYFQSESTKVIEQIQLTYNDWKRNSSNKELLKAFLDALLSYQRLLKPLNAPNDLEKDVAFYAYFDAYFTSLCGVVKLYDKVRNFMTKKPYSLEKFKLNFENSTLLDGWDVNRESDNTAILFRKEGLYYLGIMNKKYNKVFRNISSSQDEGYQKIDYKLLPGANKMLPKVFFSDKNKEYFKPNAKLLERYKAGEHKKGDNFDLDFCHELIDFFKTSIEKHQDWKHFAYQFSPTESYEDLSGFYREVEQQGYKISYKNIAASFIDTLVAEGKLYFFQIYNKDFSPYSKGTPNMHTLYWRALFDEKNLADVIYKLNGQAEIFFRKKSIEYSQEKLQKGHHHEMLKDKFAYPIIKDRRFAFDKFQFHVPITLNFKAEGNENITPKTFEYIRSNPDNIKVIGIDRGERHLLYLSLIDAEGKIVEQFTLNQIINSYNGKDHVIDYHAKLDAKEKDRDKARKEWGTVENIKELKEGYLSHVIHKIATLIIEHGAVVAMEDLNFGFKRGRFKVEKQVYQKFEKALIDKLNYLVDKKKEPHKLGGLLNALQLTSKFQSFEKMGKQNGFLFYVPAWNTSKIDPVTGFVNLFDTRYASVEKSKAFFTKFQSICYNEAKDYFELVFDYNDFTEKAKETRSEWTLCTYGERIVSFRNAEKNHQWDSKTIHLTTEFKNLFGELHGNDVKEYILEQNSVEFFKSLIYLLKITLQMRNSITGTDIDYLVSPVADEAGNFYDSRKADTSLPKDADANGAYNIARKGLMLMHRIQNAEDLKKVNLAISNRDWLRNAQGLDK

SEQ ID NO: 186 - protein, 1197 aa, synthetic construct
MLSNFTNQYQLSKTLRFELKPVGDTLKHIEKSGLIAQDEIRSQEYQEVKTIIDKYHKAFIDEALQNVVLSNLEEYEALFFERNRDEKAFEKLQAVLRKEIVAHFKQHPQYKTLFKKELIKADLKNWQELSDAEKELVSHFDNFTTYFTGFHENRANMYTDEAKHSSIAYRIIHENLPIFLTNKKLFETIKQKAPHLAQETQDALLEYLSGAIVEDMFELSYFNHLLSQTHIDLYNQMIGGVKQDSLKIQGLNEKINLYRQANGLSKRELPNLKPLHKQILSDRETLSWLPESFESDEELMQGVQAYFESEVLAFECCDGKVNLLEKLPELLHQTQDYDFSKVYFKNDLALTAASQAIFKDYRIIKEALWEVNKPKKSKDLVADEEKFFNKKNSYFSIEQIDGALNSAQLSANMMHYFQSESTKVIEQIQLTYNDWKRNSSNKELLKAFLDALLSYQRLLKPLNAPNDLEKDVAFYAYFDAYFTSLCGVVKLYDKVRNFMTKKPYSLEKFKLNFENSTLLRGWDVNRESDNTAILFRKEGLYYLGIMNKKYNKVFRNISSSQDEGYQKIDYKLLPGANKMLPKVFFSDKNKEYFKPNAKLLERYKAGEHKKGDNFDLDFCHELIDFFKTSIEKHQDWKHFAYQFSPTESYEDLSGFYREVEQQGYKISYKNIAASFIDTLVAEGKLYFFQIYNKDFSPYSKGTPNMHTLYWRALFDEKNLADVIYKLNGQAEIFFRKKSIEYSQEKLQKGHHHEMLKDKFAYPIIKDRRFAFDKFQFHVPITLNFKAEGNENITPKTFEYIRSNPDNIKVIGIDRGERHLLYLSLIDAEGKIVEQFTLNQIINSYNGKDHVIDYHAKLDAKEKDRDKARKEWGTVENIKELKEGYLSHVIHKIATLIIEHGAVVAMEDLNFGFKRGRFKVEKQVYQKFEKALIDKLNYLVDKKKEPHKLGGLLNALQLTSKFQSFEKMGKQNGFLFYVPAWNTSKIDPVTGFVNLFDTRYASVEKSKAFFTKFQSICYNEAKDYFELVFDYNDFTEKAKETRSEWTLCTYGERIVSFRNAEKNHQWDSKTIHLTTEFKNLFGELHGNDVKEYILEQNSVEFFKSLIYLLKITLQMRNSITGTDIDYLVSPVADEAGNFYDSRKADTSLPKDADANGAYNIARKGLMLMHRIQNAEDLKKVNLAISNRDWLRNAQGLDK

SEQ ID NO: 187 - protein, 1197 aa, synthetic construct
MLSNFTNQYQLSKTLRFELKPVGDTLKHIEKSGLIAQDEIRSQEYQEVKTIIDKYHKAFIDEALQNVVLSNLEEYEALFFERNRDEKAFEKLQAVLRKEIVAHFKQHPQYKTLFKKELIKADLKNWQELSDAEKELVSHFDNFTTYFTGFHRNRANMYTDEAKHSSIAYRIIHENLPIFLTNKKLFETIKQKAPHLAQETQDALLEYLSGAIVEDMFELSYFNHLLSQTHIDLYNQMIGGVKQDSLKIQGLNEKINLYRQANGLSKRELPNLKPLHKQILSDRETLSWLPESFESDEELMQGVQAYFESEVLAFECCDGKVNLLEKLPELLHQTQDYDFSKVYFKNDLALTAASQAIFKDYRIIKEALWEVNKPKKSKDLVADEEKFFNKKNSYFSIEQIDGALNSAQLSANMMHYFQSESTKVIEQIQLTYNDWKRNSSNKELLKAFLDALLSYQRLLKPLNAPNDLEKDVAFYAYFDAYFTSLCGVVKLYDKVRNFMTKKPYSLEKFKLNFENSTLLRGWDVNRESDNTAILFRKEGLYYLGIMNKKYNKVFRNISSSQDEGYQKIDYKLLPGANKMLPKVFFSDKNKEYFKPNAKLLERYKAGEHKKGDNFDLDFCHELIDFFKTSIEKHQDWKHFAYQFSPTESYEDLSGFYREVEQQGYKISYKNIAASFIDTLVAEGKLYFFQIYNKDFSPYSKGTPNMHTLYWRALFDEKNLADVIYKLNGQAEIFFRKKSIEYSQEKLQKGHHHEMLKDKFAYPIIKDRRFAFDKFQFHVPITLNFKAEGNENITPKTFEYIRSNPDNIKVIGIDRGERHLLYLSLIDAEGKIVEQFTLNQIINSYNGKDHVIDYHAKLDAKEKDRDKARKEWGTVENIKELKEGYLSHVIHKIATLIIEHGAVVAMEDLNFGFKRGRFKVEKQVYQKFEKALIDKLNYLVDKKKEPHKLGGLLNALQLTSKFQSFEKMGKQNGFLFYVPAWNTSKIDPVTGFVNLFDTRYASVEKSKAFFTKFQSICYNEAKDYFELVFDYNDFTEKAKETRSEWTLCTYGERIVSFRNAEKNHQWDSKTIHLTTEFKNLFGELHGNDVKEYILEQNSVEFFKSLIYLLKITLQMRNSITGTDIDYLVSPVADEAGNFYDSRKADTSLPKDADANGAYNIARKGLMLMHRIQNAEDLKKVNLAISNRDWLRNAQGLDK

SEQ ID NO: 188 - protein, 1197 aa, synthetic construct
MLSNFTNQYQLSKTLRFELKPVGDTLKHIEKSGLIAQDEIRSQEYQEVKTIIDKYHKAFIDEALQNVVLSNLEEYEALFFERNRDEKAFEKLQAVLRKEIVAHFKQHPQYKTLFKKELIKADLKNWQELSDAEKELVSHFDNFTTYFTGFHRNRANMYTDEAKHSSIAYRIIHENLPIFLTNKKLFETIKQKAPHLAQETQDALLEYLSGAIVEDMFELSYFNHLLSQTHIDLYNQMIGGVKQDSLKIQGLNEKIALYRQANGLSKRELPNLKPLHKQILSDRETLSWLPESFESDEELMQGVQAYFESEVLAFECCDGKVNLLEKLPELLHQTQDYDFSKVYFKNDLALTAASQAIFKDYRIIKEALWEVNKPKKSKDLVADEEKFFNKKNSYFSIEQIDGALNSAQLSANMMHYFQSESTKVIEQIQLTYNDWKRNSSNKELLKAFLDALLSYQRLLKPLNAPNDLEKDVAFYAYFDAYFTSLCGVVKLYDKVRNFMTKKPYSLEKFKLNFENSTLLDGWDVNRESDNTAILFRKEGLYYLGIMNKKYNKVFRNISSSQDEGYQKIDYKLLPGANKMLPKVFFSDKNKEYFKPNAKLLERYKAGEHKKGDNFDLDFCHELIDFFKTSIEKHQDWKHFAYQFSPTESYEDLSGFYREVEQQGYKISYKNIAASFIDTLVAEGKLYFFQIYNKDFSPYSKGTPNMHTLYWRALFDEKNLADVIYKLNGQAEIFFRKKSIEYSQEKLQKGHHHEMLKDKFAYPIIKDRRFAFDKFQFHVPITLNFKAEGNENITPKTFEYIRSNPDNIKVIGIDRGERHLLYLSLIDAEGKIVEQFTLNQIINSYNGKDHVIDYHAKLDAKEKDRDKARKEWGTVENIKELKEGYLSHVIHKIATLIIEHGAVVAMEDLNFGFKRGRFKVEKQVYQKFEKALIDKLNYLVDKKKEPHKLGGLLNALQLTSKFQSFEKMGKQNGFLFYVPAWNTSKIDPVTGFVNLFDTRYASVEKSKAFFTKFQSICYNEAKDYFELVFDYNDFTEKAKETRSEWTLCTYGERIVSFRNAEKNHQWDSKTIHLTTEFKNLFGELHGNDVKEYILEQNSVEFFKSLIYLLKITLQMRNSITGTDIDYLVSPVADEAGNFYDSRKADTSLPKDADANGAYNIARKGLMLMHRIQNAEDLKKVNLAISNRDWLRNAQGLDK

SEQ ID NO: 189 - protein, 1197 aa, synthetic construct
MLSNFTNQYQLSKTLRFELKPVGDTLKHIEKSGLIAQDEIRSQEYQEVKTIIDKYHKAFIDEALQNVVLSNLEEYEALFFERNRDEKAFEKLQAVLRKEIVAHFKQHPQYKTLFKKELIKADLKNWQELSDAEKELVSHFDNFTTYFTGFHENRANMYTDEAKHSSIAYRIIHENLPIFLTNKKLFETIKQKAPHLAQETQDALLEYLSGAIVEDMFELSYFNHLLSQTHIDLYNQMIGGVKQDSLKIQGLNEKINLYRQANGLSKRELPNLKPLHKQILSDRETLSWLPESFESDEELMQGVQAYFESEVLAFECCDGKVNLLEKLPELLHQTQDYDFSKVYFKNDLALTAASQAIFKDYRIIKEALWEVNKPKKSKDLVADEEKFFNKKNSYFSIEQIDGALNSAQLSANMMHYFQSESTKVIEQIQLTYNDWKRNSSNKELLKAFLDALLSYQRLLKPLNAPNDLEKDVAFYAYFDAYFTSLCGVVKLYDKVRNFMTKKPYSLEKFKLNFERSTLLDGWDVNKESDNTAILFRKEGLYYLGIMNKKYNKVFRNISSSQDEGYQKIDYKLLPGANKMLPKVFFSDKNKEYFKPNAKLLERYKAGEHKKGDNFDLDFCHELIDFFKTSIEKHQDWKHFAYQFSPTESYEDLSGFYREVEQQGYKISYKNIAASFIDTLVAEGKLYFFQIYNKDFSPYSKGTPNMHTLYWRALFDEKNLADVIYKLNGQAEIFFRKKSIEYSQEKLQKGHHHEMLKDKFAYPIIKDRRFAFDKFLFHVPITLNFKAEGNENITPKTFEYIRSNPDNIKVIGIDRGERHLLYLSLIDAEGKIVEQFTLNQIINSYNGKDHVIDYHAKLDAKEKDRDKARKEWGTVENIKELKEGYLSHVIHKIATLIIEHGAVVAMEDLNFGFKRGRFKVEKQVYQKFEKALIDKLNYLVDKKKEPHKLGGLLNALQLTSKFQSFEKMGKQNGFLFYVPAWNTSKIDPVTGFVNLFDTRYASVEKSKAFFTKFQSICYNEAKDYFELVFDYNDFTEKAKETRSEWTLCTYGERIVSFRNAEKNHQWDSKTIHLTTEFKNLFGELHGNDVKEYILEQNSVEFFKSLIYLLKITLQMRNSITGTDIDYLVSPVADEAGNFYDSRKADTSLPKDADANGAYNIARKGLMLMHRIQNAEDLKKVNLAISNRDWLRNAQGLDK

SEQ ID NO: 190 - protein, 1227 aa, synthetic construct
MKEQFINCYPLSKTLQFSLIPVGKTDDNFNKKLLLERDKQRAENYEKVKGYIDRFHKEYIESVLFNARVEKIDEYADLYWKSNKDDSDAKAMESLENDMRKQISKQLKSNARYKRLFGKELICEDLPSFLTDKKERETVECFRSFTTYFKGFNRNRENMYSSDEKSTAIAYRCINDNLPRFLDNVKSFQKVFDNLSDETITKLNTDLYNTFGRNIEDIFSVDYFEFVLAQSGIDIYNSMIGGYTCSDGTKIQGLNECINLYNQQAAKNEKSKRLPLMKQLYKQILSEKDSVSFIPEKFNSDNEVLLSIEDYYSSHIGDLDLLTELLQSLNTYNANGIFVKSGAAVSDISNGAFNSWNVLRLAWNEKYEALHPVTSKTKLDNYIEKRDKIYKAIKSFSLFELQSLGNENGNEITDWYISSSKECNSKIKEAYLQARELLKSDYEKSYNKRLSKNGKATELVKNLLDAIKNFQHLVKSLNGTGKEENKDELFYGKLTSYYDSIADIDRLYDKVRNYITQKPYSKDKIKLNFDNPQLLGGWDKNKESDYRTVLLRKDDFYYLAVMDKSHSKAFVDAPNITSEDEDYYEKMEYKLLPGPNKMLPKVFFAAKNIDTFQPSDRILDIRKRESFKKGATFNKSECHEFIDYFKNSIEKHDDWSQFGFEFTPTENYNDISEFYREISDQGYSVSFNKISKSYVDELVDNGYIYLFQIYNKDFSKYSKGTPNLHTLYFKMLFDERNLSNVVYKLNGEAEMFYREASINDKEKITHQANQPIKNKNPDNEKKESTFEYDIIKDKRFTKRQFSLHVPITINFKAHGQEFLNYDVRKAVKYKDDNYVIGIDRGERNLIYISVIDSNGKIVEQISLNEIISDNGHRVDYQKLLDTKEKERDKARKNWTSVENIKELKEGYISQVVHKICELVVKYDAVIAMEDLNFGFKRGRFPVEKQVYQKFENMLISKLNLLIDKKADPTEDGGLLRAYQLTNKFDGVNKGKQNGIIFYVPAWDTSKIDPVTGFVNLLKPKYTSVSEAKKLFETIDDIKYNANTDMFEFCIDYGKFPRCNSDYKNTWTVCTNSSRILTCRNKEKNNMWDNKQIVLTDKFKSLFNEFGIDYKGNLKTSILSISNADFYRRLIKLLSLTLQMRNSITGSTLPEDDYLISPVANDRGEFYDSRNYKGTNAALPCDADANGAYNIARKALWAINVLKSTPDDMLNKANLSITNAEWLEYTQK

SEQ ID NO: 191 - protein, 1227 aa, synthetic construct
MKEQFINCYPLSKTLQFSLIPVGKTDDNFNKKLLLERDKQRAENYEKVKGYIDRFHKEYIESVLFNARVEKIDEYADLYWKSNKDDSDAKAMESLENDMRKQISKQLKSNARYKRLFGKELICEDLPSFLTDKKERETVECFRSFTTYFKGFNTNRENMYSSDEKSTAIAYRCINDNLPRFLDNVKSFQKVFDNLSDETITKLNTDLYNTFGRNIEDIFSVDYFEFVLAQSGIDIYNSMIGGYTCSDGTKIQGLNECINLYNQQAAKNEKSKRLPLMKQLYKQILSEKDSVSFIPEKFNSDNEVLLSIEDYYSSHIGDLDLLTELLQSLNTYNANGIFVKSGAAVSDISNGAFNSWNVLRLAWNEKYEALHPVTSKTKLDNYIEKRDKIYKAIKSFSLFELQSLGNENGNEITDWYISSSKECNSKIKEAYLQARELLKSDYEKSYNKRLSKNGKATELVKNLLDAIKNFQHLVKSLNGTGKEENKDELFYGKLTSYYDSIADIDRLYDKVRNYITQKPYSKDKIKLNFDNPQLLRGWDKNKESDYRTVLLRKDDFYYLAVMDKSHSKAFVDAPNITSEDEDYYEKMEYKLLPGPNKMLPKVFFAAKNIDTFQPSDRILDIRKRESFKKGATFNKSECHEFIDYFKNSIEKHDDWSQFGFEFTPTENYNDISEFYREISDQGYSVSFNKISKSYVDELVDNGYIYLFQIYNKDFSKYSKGTPNLHTLYFKMLFDERNLSNVVYKLNGEAEMFYREASINDKEKITHQANQPIKNKNPDNEKKESTFEYDIIKDKRFTKRQFSLHVPITINFKAHGQEFLNYDVRKAVKYKDDNYVIGIDRGERNLIYISVIDSNGKIVEQISLNEIISDNGHRVDYQKLLDTKEKERDKARKNWTSVENIKELKEGYISQVVHKICELVVKYDAVIAMEDLNFGFKRGRFPVEKQVYQKFENMLISKLNLLIDKKADPTEDGGLLRAYQLTNKFDGVNKGKQNGIIFYVPAWDTSKIDPVTGFVNLLKPKYTSVSEAKKLFETIDDIKYNANTDMFEFCIDYGKFPRCNSDYKNTWTVCTNSSRILTCRNKEKNNMWDNKQIVLTDKFKSLFNEFGIDYKGNLKTSILSISNADFYRRLIKLLSLTLQMRNSITGSTLPEDDYLISPVANDRGEFYDSRNYKGTNAALPCDADANGAYNIARKALWAINVLKSTPDDMLNKANLSITNAEWLEYTQK

SEQ ID NO: 192 - protein, 1227 aa, synthetic construct
MKEQFINCYPLSKTLQFSLIPVGKTDDNFNKKLLLERDKQRAENYEKVKGYIDRFHKEYIESVLFNARVEKIDEYADLYWKSNKDDSDAKAMESLENDMRKQISKQLKSNARYKRLFGKELICEDLPSFLTDKKERETVECFRSFTTYFKGFNTNRENMYSSDEKSTAIAYRCINDNLPRFLDNVKSFQKVFDNLSDETITKLNTDLYNTFGRNIEDIFSVDYFEFVLAQSGIDIYNSMIGGYTCSDGTKIQGLNECINLYNQQAAKNEKSKRLPLMKQLYKQILSEKDSVSFIPEKFNSDNEVLLSIEDYYSSHIGDLDLLTELLQSLNTYNANGIFVKSGAAVSDISNGAFNSWNVLRLAWNEKYEALHPVTSKTKLDNYIEKRDKIYKAIKSFSLFELQSLGNENGNEITDWYISSSKECNSKIKEAYLQARELLKSDYEKSYNKRLSKNGKATELVKNLLDAIKNFQHLVKSLNGTGKEENKDELFYGKLTSYYDSIADIDRLYDKVRNYITQKPYSKDKIKLNFDNPQLLGGWDKNRESDYRTVLLRKDDFYYLAVMDKSHSKAFVDAPNITSEDEDYYEKMEYKLLPGPNKMLPKVFFAAKNIDTFQPSDRILDIRKRESFKKGATFNKSECHEFIDYFKNSIEKHDDWSQFGFEFTPTENYNDISEFYREISDQGYSVSFNKISKSYVDELVDNGYIYLFQIYNKDFSKYSKGTPNLHTLYFKMLFDERNLSNVVYKLNGEAEMFYREASINDKEKITHQANQPIKNKNPDNEKKESTFEYDIIKDKRFTKRQFSLHVPITINFKAHGQEFLNYDVRKAVKYKDDNYVIGIDRGERNLIYISVIDSNGKIVEQISLNEIISDNGHRVDYQKLLDTKEKERDKARKNWTSVENIKELKEGYISQVVHKICELVVKYDAVIAMEDLNFGFKRGRFPVEKQVYQKFENMLISKLNLLIDKKADPTEDGGLLRAYQLTNKFDGVNKGKQNGIIFYVPAWDTSKIDPVTGFVNLLKPKYTSVSEAKKLFETIDDIKYNANTDMFEFCIDYGKFPRCNSDYKNTWTVCTNSSRILTCRNKEKNNMWDNKQIVLTDKFKSLFNEFGIDYKGNLKTSILSISNADFYRRLIKLLSLTLQMRNSITGSTLPEDDYLISPVANDRGEFYDSRNYKGTNAALPCDADANGAYNIARKALWAINVLKSTPDDMLNKANLSITNAEWLEYTQK

SEQ ID NO: 193 - protein, 1227 aa, synthetic construct
MKEQFINCYPLSKTLQFSLIPVGKTDDNFNKKLLLERDKQRAENYEKVKGYIDRFHKEYIESVLFNARVEKIDEYADLYWKSNKDDSDAKAMESLENDMRKQISKQLKSNARYKRLFGKELICEDLPSFLTDKKERETVECFRSFTTYFKGFNRNRENMYSSDEKSTAIAYRCINDNLPRFLDNVKSFQKVFDNLSDETITKLNTDLYNTFGRNIEDIFSVDYFEFVLAQSGIDIYNSMIGGYTCSDGTKIQGLNECINLYNQQAAKNEKSKRLPLMKQLYKQILSEKDSVSFIPEKFNSDNEVLLSIEDYYSSHIGDLDLLTELLQSLNTYNANGIFVKSGAAVSDISNGAFNSWNVLRLAWNEKYEALHPVTSKTKLDNYIEKRDKIYKAIKSFSLFELQSLGNENGNEITDWYISSSKECNSKIKEAYLQARELLKSDYEKSYNKRLSKNGKATELVKNLLDAIKNFQHLVKSLNGTGKEENKDELFYGKLTSYYDSIADIDRLYDKVRNYITQKPYSKDKIKLNFDNPQLLRGWDKNKESDYRTVLLRKDDFYYLAVMDKSHSKAFVDAPNITSEDEDYYEKMEYKLLPGPNKMLPKVFFAAKNIDTFQPSDRILDIRKRESFKKGATFNKSECHEFIDYFKNSIEKHDDWSQFGFEFTPTENYNDISEFYREISDQGYSVSFNKISKSYVDELVDNGYIYLFQIYNKDFSKYSKGTPNLHTLYFKMLFDERNLSNVVYKLNGEAEMFYREASINDKEKITHQANQPIKNKNPDNEKKESTFEYDIIKDKRFTKRQFSLHVPITINFKAHGQEFLNYDVRKAVKYKDDNYVIGIDRGERNLIYISVIDSNGKIVEQISLNEIISDNGHRVDYQKLLDTKEKERDKARKNWTSVENIKELKEGYISQVVHKICELVVKYDAVIAMEDLNFGFKRGRFPVEKQVYQKFENMLISKLNLLIDKKADPTEDGGLLRAYQLTNKFDGVNKGKQNGIIFYVPAWDTSKIDPVTGFVNLLKPKYTSVSEAKKLFETIDDIKYNANTDMFEFCIDYGKFPRCNSDYKNTWTVCTNSSRILTCRNKEKNNMWDNKQIVLTDKFKSLFNEFGIDYKGNLKTSILSISNADFYRRLIKLLSLTLQMRNSITGSTLPEDDYLISPVANDRGEFYDSRNYKGTNAALPCDADANGAYNIARKALWAINVLKSTPDDMLNKANLSITNAEWLEYTQK

SEQ ID NO: 194 - protein, 1227 aa, synthetic construct
MKEQFINCYPLSKTLQFSLIPVGKTDDNFNKKLLLERDKQRAENYEKVKGYIDRFHKEYIESVLFNARVEKIDEYADLYWKSNKDDSDAKAMESLENDMRKQISKQLKSNARYKRLFGKELICEDLPSFLTDKKERETVECFRSFTTYFKGFNRNRENMYSSDEKSTAIAYRCINDNLPRFLDNVKSFQKVFDNLSDETITKLNTDLYNTFGRNIEDIFSVDYFEFVLAQSGIDIYNSMIGGYTCSDGTKIQGLNECINLYNQQAAKNEKSKRLPLMKQLYKQILSEKDSVSFIPEKFNSDNEVLLSIEDYYSSHIGDLDLLTELLQSLNTYNANGIFVKSGAAVSDISNGAFNSWNVLRLAWNEKYEALHPVTSKTKLDNYIEKRDKIYKAIKSFSLFELQSLGNENGNEITDWYISSSKECNSKIKEAYLQARELLKSDYEKSYNKRLSKNGKATELVKNLLDAIKNFQHLVKSLNGTGKEENKDELFYGKLTSYYDSIADIDRLYDKVRNYITQKPYSKDKIKLNFDNPQLLGGWDKNRESDYRTVLLRKDDFYYLAVMDKSHSKAFVDAPNITSEDEDYYEKMEYKLLPGPNKMLPKVFFAAKNIDTFQPSDRILDIRKRESFKKGATFNKSECHEFIDYFKNSIEKHDDWSQFGFEFTPTENYNDISEFYREISDQGYSVSFNKISKSYVDELVDNGYIYLFQIYNKDFSKYSKGTPNLHTLYFKMLFDERNLSNVVYKLNGEAEMFYREASINDKEKITHQANQPIKNKNPDNEKKESTFEYDIIKDKRFTKRQFSLHVPITINFKAHGQEFLNYDVRKAVKYKDDNYVIGIDRGERNLIYISVIDSNGKIVEQISLNEIISDNGHRVDYQKLLDTKEKERDKARKNWTSVENIKELKEGYISQVVHKICELVVKYDAVIAMEDLNFGFKRGRFPVEKQVYQKFENMLISKLNLLIDKKADPTEDGGLLRAYQLTNKFDGVNKGKQNGIIFYVPAWDTSKIDPVTGFVNLLKPKYTSVSEAKKLFETIDDIKYNANTDMFEFCIDYGKFPRCNSDYKNTWTVCTNSSRILTCRNKEKNNMWDNKQIVLTDKFKSLFNEFGIDYKGNLKTSILSISNADFYRRLIKLLSLTLQMRNSITGSTLPEDDYLISPVANDRGEFYDSRNYKGTNAALPCDADANGAYNIARKALWAINVLKSTPDDMLNKANLSITNAEWLEYTQK

SEQ ID NO: 195 - protein, 1227 aa, synthetic construct
MKEQFINCYPLSKTLQFSLIPVGKTDDNFNKKLLLERDKQRAENYEKVKGYIDRFHKEYIESVLFNARVEKIDEYADLYWKSNKDDSDAKAMESLENDMRKQISKQLKSNARYKRLFGKELICEDLPSFLTDKKERETVECFRSFTTYFKGFNTNRENMYSSDEKSTAIAYRCINDNLPRFLDNVKSFQKVFDNLSDETITKLNTDLYNTFGRNIEDIFSVDYFEFVLAQSGIDIYNSMIGGYTCSDGTKIQGLNECINLYNQQAAKNEKSKRLPLMKQLYKQILSEKDSVSFIPEKFNSDNEVLLSIEDYYSSHIGDLDLLTELLQSLNTYNANGIFVKSGAAVSDISNGAFNSWNVLRLAWNEKYEALHPVTSKTKLDNYIEKRDKIYKAIKSFSLFELQSLGNENGNEITDWYISSSKECNSKIKEAYLQARELLKSDYEKSYNKRLSKNGKATELVKNLLDAIKNFQHLVKSLNGTGKEENKDELFYGKLTSYYDSIADIDRLYDKVRNYITQKPYSKDKIKLNFDNPQLLRGWDKNRESDYRTVLLRKDDFYYLAVMDKSHSKAFVDAPNITSEDEDYYEKMEYKLLPGPNKMLPKVFFAAKNIDTFQPSDRILDIRKRESFKKGATFNKSECHEFIDYFKNSIEKHDDWSQFGFEFTPTENYNDISEFYREISDQGYSVSFNKISKSYVDELVDNGYIYLFQIYNKDFSKYSKGTPNLHTLYFKMLFDERNLSNVVYKLNGEAEMFYREASINDKEKITHQANQPIKNKNPDNEKKESTFEYDIIKDKRFTKRQFSLHVPITINFKAHGQEFLNYDVRKAVKYKDDNYVIGIDRGERNLIYISVIDSNGKIVEQISLNEIISDNGHRVDYQKLLDTKEKERDKARKNWTSVENIKELKEGYISQVVHKICELVVKYDAVIAMEDLNFGFKRGRFPVEKQVYQKFENMLISKLNLLIDKKADPTEDGGLLRAYQLTNKFDGVNKGKQNGIIFYVPAWDTSKIDPVTGFVNLLKPKYTSVSEAKKLFETIDDIKYNANTDMFEFCIDYGKFPRCNSDYKNTWTVCTNSSRILTCRNKEKNNMWDNKQIVLTDKFKSLFNEFGIDYKGNLKTSILSISNADFYRRLIKLLSLTLQMRNSITGSTLPEDDYLISPVANDRGEFYDSRNYKGTNAALPCDADANGAYNIARKALWAINVLKSTPDDMLNKANLSITNAEWLEYTQK

SEQ ID NO: 196 - protein, 1227 aa, synthetic construct
MKEQFINCYPLSKTLQFSLIPVGKTDDNFNKKLLLERDKQRAENYEKVKGYIDRFHKEYIESVLFNARVEKIDEYADLYWKSNKDDSDAKAMESLENDMRKQISKQLKSNARYKRLFGKELICEDLPSFLTDKKERETVECFRSFTTYFKGFNRNRENMYSSDEKSTAIAYRCINDNLPRFLDNVKSFQKVFDNLSDETITKLNTDLYNTFGRNIEDIFSVDYFEFVLAQSGIDIYNSMIGGYTCSDGTKIQGLNECINLYNQQAAKNEKSKRLPLMKQLYKQILSEKDSVSFIPEKFNSDNEVLLSIEDYYSSHIGDLDLLTELLQSLNTYNANGIFVKSGAAVSDISNGAFNSWNVLRLAWNEKYEALHPVTSKTKLDNYIEKRDKIYKAIKSFSLFELQSLGNENGNEITDWYISSSKECNSKIKEAYLQARELLKSDYEKSYNKRLSKNGKATELVKNLLDAIKNFQHLVKSLNGTGKEENKDELFYGKLTSYYDSIADIDRLYDKVRNYITQKPYSKDKIKLNFDNPQLLRGWDKNRESDYRTVLLRKDDFYYLAVMDKSHSKAFVDAPNITSEDEDYYEKMEYKLLPGPNKMLPKVFFAAKNIDTFQPSDRILDIRKRESFKKGATFNKSECHEFIDYFKNSIEKHDDWSQFGFEFTPTENYNDISEFYREISDQGYSVSFNKISKSYVDELVDNGYIYLFQIYNKDFSKYSKGTPNLHTLYFKMLFDERNLSNVVYKLNGEAEMFYREASINDKEKITHQANQPIKNKNPDNEKKESTFEYDIIKDKRFTKRQFSLHVPITINFKAHGQEFLNYDVRKAVKYKDDNYVIGIDRGERNLIYISVIDSNGKIVEQISLNEIISDNGHRVDYQKLLDTKEKERDKARKNWTSVENIKELKEGYISQVVHKICELVVKYDAVIAMEDLNFGFKRGRFPVEKQVYQKFENMLISKLNLLIDKKADPTEDGGLLRAYQLTNKFDGVNKGKQNGIIFYVPAWDTSKIDPVTGFVNLLKPKYTSVSEAKKLFETIDDIKYNANTDMFEFCIDYGKFPRCNSDYKNTWTVCTNSSRILTCRNKEKNNMWDNKQIVLTDKFKSLFNEFGIDYKGNLKTSILSISNADFYRRLIKLLSLTLQMRNSITGSTLPEDDYLISPVANDRGEFYDSRNYKGTNAALPCDADANGAYNIARKALWAINVLKSTPDDMLNKANLSITNAEWLEYTQK

SEQ ID NO: 197 - protein, 1227 aa, synthetic construct
MKEQFINCYPLSKTLQFSLIPVGKTDDNFNKKLLLERDKQRAENYEKVKGYIDRFHKEYIESVLFNARVEKIDEYADLYWKSNKDDSDAKAMESLENDMRKQISKQLKSNARYKRLFGKELICEDLPSFLTDKKERETVECFRSFTTYFKGFNRNRENMYSSDEKSTAIAYRCINDNLPRFLDNVKSFQKVFDNLSDETITKLNTDLYNTFGRNIEDIFSVDYFEFVLAQSGIDIYNSMIGGYTCSDGTKIQGLNECIALYNQQAAKNEKSKRLPLMKQLYKQILSEKDSVSFIPEKFNSDNEVLLSIEDYYSSHIGDLDLLTELLQSLNTYNANGIFVKSGAAVSDISNGAFNSWNVLRLAWNEKYEALHPVTSKTKLDNYIEKRDKIYKAIKSFSLFELQSLGNENGNEITDWYISSSKECNSKIKEAYLQARELLKSDYEKSYNKRLSKNGKATELVKNLLDAIKNFQHLVKSLNGTGKEENKDELFYGKLTSYYDSIADIDRLYDKVRNYITQKPYSKDKIKLNFDNPQLLGGWDKNRESDYRTVLLRKDDFYYLAVMDKSHSKAFVDAPNITSEDEDYYEKMEYKLLPGPNKMLPKVFFAAKNIDTFQPSDRILDIRKRESFKKGATFNKSECHEFIDYFKNSIEKHDDWSQFGFEFTPTENYNDISEFYREISDQGYSVSFNKISKSYVDELVDNGYIYLFQIYNKDFSKYSKGTPNLHTLYFKMLFDERNLSNVVYKLNGEAEMFYREASINDKEKITHQANQPIKNKNPDNEKKESTFEYDIIKDKRFTKRQFSLHVPITINFKAHGQEFLNYDVRKAVKYKDDNYVIGIDRGERNLIYISVIDSNGKIVEQISLNEIISDNGHRVDYQKLLDTKEKERDKARKNWTSVENIKELKEGYISQVVHKICELVVKYDAVIAMEDLNFGFKRGRFPVEKQVYQKFENMLISKLNLLIDKKADPTEDGGLLRAYQLTNKFDGVNKGKQNGIIFYVPAWDTSKIDPVTGFVNLLKPKYTSVSEAKKLFETIDDIKYNANTDMFEFCIDYGKFPRCNSDYKNTWTVCTNSSRILTCRNKEKNNMWDNKQIVLTDKFKSLFNEFGIDYKGNLKTSILSISNADFYRRLIKLLSLTLQMRNSITGSTLPEDDYLISPVANDRGEFYDSRNYKGTNAALPCDADANGAYNIARKALWAINVLKSTPDDMLNKANLSITNAEWLEYTQK

SEQ ID NO: 198 - protein, 1227 aa, synthetic construct
MKEQFINCYPLSKTLQFSLIPVGKTDDNFNKKLLLERDKQRAENYEKVKGYIDRFHKEYIESVLFNARVEKIDEYADLYWKSNKDDSDAKAMESLENDMRKQISKQLKSNARYKRLFGKELICEDLPSFLTDKKERETVECFRSFTTYFKGFNTNRENMYSSDEKSTAIAYRCINDNLPRFLDNVKSFQKVFDNLSDETITKLNTDLYNTFGRNIEDIFSVDYFEFVLAQSGIDIYNSMIGGYTCSDGTKIQGLNECINLYNQQAAKNEKSKRLPLMKQLYKQILSEKDSVSFIPEKFNSDNEVLLSIEDYYSSHIGDLDLLTELLQSLNTYNANGIFVKSGAAVSDISNGAFNSWNVLRLAWNEKYEALHPVTSKTKLDNYIEKRDKIYKAIKSFSLFELQSLGNENGNEITDWYISSSKECNSKIKEAYLQARELLKSDYEKSYNKRLSKNGKATELVKNLLDAIKNFQHLVKSLNGTGKEENKDELFYGKLTSYYDSIADIDRLYDKVRNYITQKPYSKDKIKLNFDRPQLLGGWDKNKESDYRTVLLRKDDFYYLAVMDKSHSKAFVDAPNITSEDEDYYEKMEYKLLPGPNKMLPKVFFAAKNIDTFQPSDRILDIRKRESFKKGATFNKSECHEFIDYFKNSIEKHDDWSQFGFEFTPTENYNDISEFYREISDQGYSVSFNKISKSYVDELVDNGYIYLFQIYNKDFSKYSKGTPNLHTLYFKMLFDERNLSNVVYKLNGEAEMFYREASINDKEKITHQANQPIKNKNPDNEKKESTFEYDIIKDKRFTKRQFLLHVPITINFKAHGQEFLNYDVRKAVKYKDDNYVIGIDRGERNLIYISVIDSNGKIVEQISLNEIISDNGHRVDYQKLLDTKEKERDKARKNWTSVENIKELKEGYISQVVHKICELVVKYDAVIAMEDLNFGFKRGRFPVEKQVYQKFENMLISKLNLLIDKKADPTEDGGLLRAYQLTNKFDGVNKGKQNGIIFYVPAWDTSKIDPVTGFVNLLKPKYTSVSEAKKLFETIDDIKYNANTDMFEFCIDYGKFPRCNSDYKNTWTVCTNSSRILTCRNKEKNNMWDNKQIVLTDKFKSLFNEFGIDYKGNLKTSILSISNADFYRRLIKLLSLTLQMRNSITGSTLPEDDYLISPVANDRGEFYDSRNYKGTNAALPCDADANGAYNIARKALWAINVLKSTPDDMLNKANLSITNAEWLEYTQK

SEQ ID NO: 199 - protein, 1236 aa, synthetic construct
MKQLSELTGLYSLSKTLRFELKPIGKTLEHIESKGLIAQDEQRAEEYKKVKDIIDRYHKKFITMCLSDFKFEQNSLETYVALAEDTNRDEKAFDEIKKTLRKQIVETFKKGGSYSDLFKKELILKHLPEFVSDEEEKKMIANFSKFTTYFNGFNRNRKNMYSDEDKSTAIAYRLIHENLPMFVNNMKSFSRIADSDVHQHFTEIESSFSEYLGVEHLEEMFQLDYFSETLTQEQIAVYNHIIGGRALEDGTKLKGINEYVNLYNQQHKDSRLPLLKPLYKMILSDRVALPWFPSEEFASDEEMIRAINETYNTLKEVLSIDGDSSLRSLLLNISSYDIKHIYIANDLGLTDISQQMFGQYDVYTSAIKQELREKNAPTAKERRNPELYDERINKLFKSARSFSIEHLNSLVDSEHTIQNYYKHLGAYDRNGEQRVNLFTQIEMAYVAAKEILSGKHGNINQSDAETAIIKGLLDAFKSMQRFIKPLLGNGEEADKDNEFDAKLREAWNTLDIVTPLYNKVRNWLTRKPYSTEKIKLNFENAQLLGGWDVNKEPDCTSILLRKNGLYYLAIMNKKYNHAFDIDNLPNDGKCYEKIDYKLLPGANKMLPKVFFSKSRIKEFAPSEIIVNAYKKGSHKKGANFSISDCRRLIDFFKASISKHEDWKNFPFSFSSTETYEDISGFYREVEQQGYMLGFRNVSESFIKQLVDEGKLYLFQIWNKDFSDFSKGTPNMHTLYWKMLFDEHNLANVVYKLNGQAEVFYRKSSLDVANTTIHKAHQPIMNKNQENKKRQSTFDYDIIKNRRYTVDKFQFHVPISINFKATGRDNVNDNVLDIIRNGGIKHIIGIDRGERHLLYISLIDLKGNIVKQMTLNDIVNEYNGNTYATNYKNLLAEREGNRTEARKNWKKIENIKDIKKGYLSQVVHIISKMMVEYNAIVVLEDLNMGFMRGRQKIERSVYEQFEKMLIDKLNYYVDKQKNSDEAGGLLHALQLTSKFESFKKLGKQSGCLFYIPAWNTSKIDPVTGFVNLFDTRYTNADQARKFFSQFDSIRYNADKDWFEFAFDYNKFTAKAKDTRTKWTLCTYGTRIRTFRNPARLNQWDNEEVVLTDEFKKAFANADVNIHGNLKEAICSLQDKKYLEPLMHLMKLLLQMRNSITNSEVDYLLSPVADENGTFYDSRVCSASLPKDADANGAYNIARKGLWAIHQIQETPAGDRPNLAIKNKDWLNFAQQKPYMNE

SEQ ID NO: 200 - protein, 1236 aa, synthetic construct
MKQLSELTGLYSLSKTLRFELKPIGKTLEHIESKGLIAQDEQRAEEYKKVKDIIDRYHKKFITMCLSDFKFEQNSLETYVALAEDTNRDEKAFDEIKKTLRKQIVETFKKGGSYSDLFKKELILKHLPEFVSDEEEKKMIANFSKFTTYFNGFNENRKNMYSDEDKSTAIAYRLIHENLPMFVNNMKSFSRIADSDVHQHFTEIESSFSEYLGVEHLEEMFQLDYFSETLTQEQIAVYNHIIGGRALEDGTKLKGINEYVNLYNQQHKDSRLPLLKPLYKMILSDRVALPWFPSEEFASDEEMIRAINETYNTLKEVLSIDGDSSLRSLLLNISSYDIKHIYIANDLGLTDISQQMFGQYDVYTSAIKQELREKNAPTAKERRNPELYDERINKLFKSARSFSIEHLNSLVDSEHTIQNYYKHLGAYDRNGEQRVNLFTQIEMAYVAAKEILSGKHGNINQSDAETAIIKGLLDAFKSMQRFIKPLLGNGEEADKDNEFDAKLREAWNTLDIVTPLYNKVRNWLTRKPYSTEKIKLNFENAQLLRGWDVNKEPDCTSILLRKNGLYYLAIMNKKYNHAFDIDNLPNDGKCYEKIDYKLLPGANKMLPKVFFSKSRIKEFAPSEIIVNAYKKGSHKKGANFSISDCRRLIDFFKASISKHEDWKNFPFSFSSTETYEDISGFYREVEQQGYMLGFRNVSESFIKQLVDEGKLYLFQIWNKDFSDFSKGTPNMHTLYWKMLFDEHNLANVVYKLNGQAEVFYRKSSLDVANTTIHKAHQPIMNKNQENKKRQSTFDYDIIKNRRYTVDKFQFHVPISINFKATGRDNVNDNVLDIIRNGGIKHIIGIDRGERHLLYISLIDLKGNIVKQMTLNDIVNEYNGNTYATNYKNLLAEREGNRTEARKNWKKIENIKDIKKGYLSQVVHIISKMMVEYNAIVVLEDLNMGFMRGRQKIERSVYEQFEKMLIDKLNYYVDKQKNSDEAGGLLHALQLTSKFESFKKLGKQSGCLFYIPAWNTSKIDPVTGFVNLFDTRYTNADQARKFFSQFDSIRYNADKDWFEFAFDYNKFTAKAKDTRTKWTLCTYGTRIRTFRNPARLNQWDNEEVVLTDEFKKAFANADVNIHGNLKEAICSLQDKKYLEPLMHLMKLLLQMRNSITNSEVDYLLSPVADENGTFYDSRVCSASLPKDADANGAYNIARKGLWAIHQIQETPAGDRPNLAIKNKDWLNFAQQKPYMNE

SEQ ID NO: 201 - protein, 1236 aa, synthetic construct
MKQLSELTGLYSLSKTLRFELKPIGKTLEHIESKGLIAQDEQRAEEYKKVKDIIDRYHKKFITMCLSDFKFEQNSLETYVALAEDTNRDEKAFDEIKKTLRKQIVETFKKGGSYSDLFKKELILKHLPEFVSDEEEKKMIANFSKFTTYFNGFNENRKNMYSDEDKSTAIAYRLIHENLPMFVNNMKSFSRIADSDVHQHFTEIESSFSEYLGVEHLEEMFQLDYFSETLTQEQIAVYNHIIGGRALEDGTKLKGINEYVNLYNQQHKDSRLPLLKPLYKMILSDRVALPWFPSEEFASDEEMIRAINETYNTLKEVLSIDGDSSLRSLLLNISSYDIKHIYIANDLGLTDISQQMFGQYDVYTSAIKQELREKNAPTAKERRNPELYDERINKLFKSARSFSIEHLNSLVDSEHTIQNYYKHLGAYDRNGEQRVNLFTQIEMAYVAAKEILSGKHGNINQSDAETAIIKGLLDAFKSMQRFIKPLLGNGEEADKDNEFDAKLREAWNTLDIVTPLYNKVRNWLTRKPYSTEKIKLNFENAQLLGGWDVNREPDCTSILLRKNGLYYLAIMNKKYNHAFDIDNLPNDGKCYEKIDYKLLPGANKMLPKVFFSKSRIKEFAPSEIIVNAYKKGSHKKGANFSISDCRRLIDFFKASISKHEDWKNFPFSFSSTETYEDISGFYREVEQQGYMLGFRNVSESFIKQLVDEGKLYLFQIWNKDFSDFSKGTPNMHTLYWKMLFDEHNLANVVYKLNGQAEVFYRKSSLDVANTTIHKAHQPIMNKNQENKKRQSTFDYDIIKNRRYTVDKFQFHVPISINFKATGRDNVNDNVLDIIRNGGIKHIIGIDRGERHLLYISLIDLKGNIVKQMTLNDIVNEYNGNTYATNYKNLLAEREGNRTEARKNWKKIENIKDIKKGYLSQVVHIISKMMVEYNAIVVLEDLNMGFMRGRQKIERSVYEQFEKMLIDKLNYYVDKQKNSDEAGGLLHALQLTSKFESFKKLGKQSGCLFYIPAWNTSKIDPVTGFVNLFDTRYTNADQARKFFSQFDSIRYNADKDWFEFAFDYNKFTAKAKDTRTKWTLCTYGTRIRTFRNPARLNQWDNEEVVLTDEFKKAFANADVNIHGNLKEAICSLQDKKYLEPLMHLMKLLLQMRNSITNSEVDYLLSPVADENGTFYDSRVCSASLPKDADANGAYNIARKGLWAIHQIQETPAGDRPNLAIKNKDWLNFAQQKPYMNE

SEQ ID NO: 202 - protein, 1236 aa, synthetic construct
MKQLSELTGLYSLSKTLRFELKPIGKTLEHIESKGLIAQDEQRAEEYKKVKDIIDRYHKKFITMCLSDFKFEQNSLETYVALAEDTNRDEKAFDEIKKTLRKQIVETFKKGGSYSDLFKKELILKHLPEFVSDEEEKKMIANFSKFTTYFNGFNRNRKNMYSDEDKSTAIAYRLIHENLPMFVNNMKSFSRIADSDVHQHFTEIESSFSEYLGVEHLEEMFQLDYFSETLTQEQIAVYNHIIGGRALEDGTKLKGINEYVNLYNQQHKDSRLPLLKPLYKMILSDRVALPWFPSEEFASDEEMIRAINETYNTLKEVLSIDGDSSLRSLLLNISSYDIKHIYIANDLGLTDISQQMFGQYDVYTSAIKQELREKNAPTAKERRNPELYDERINKLFKSARSFSIEHLNSLVDSEHTIQNYYKHLGAYDRNGEQRVNLFTQIEMAYVAAKEILSGKHGNINQSDAETAIIKGLLDAFKSMQRFIKPLLGNGEEADKDNEFDAKLREAWNTLDIVTPLYNKVRNWLTRKPYSTEKIKLNFENAQLLRGWDVNKEPDCTSILLRKNGLYYLAIMNKKYNHAFDIDNLPNDGKCYEKIDYKLLPGANKMLPKVFFSKSRIKEFAPSEIIVNAYKKGSHKKGANFSISDCRRLIDFFKASISKHEDWKNFPFSFSSTETYEDISGFYREVEQQGYMLGFRNVSESFIKQLVDEGKLYLFQIWNKDFSDFSKGTPNMHTLYWKMLFDEHNLANVVYKLNGQAEVFYRKSSLDVANTTIHKAHQPIMNKNQENKKRQSTFDYDIIKNRRYTVDKFQFHVPISINFKATGRDNVNDNVLDIIRNGGIKHIIGIDRGERHLLYISLIDLKGNIVKQMTLNDIVNEYNGNTYATNYKNLLAEREGNRTEARKNWKKIENIKDIKKGYLSQVVHIISKMMVEYNAIVVLEDLNMGFMRGRQKIERSVYEQFEKMLIDKLNYYVDKQKNSDEAGGLLHALQLTSKFESFKKLGKQSGCLFYIPAWNTSKIDPVTGFVNLFDTRYTNADQARKFFSQFDSIRYNADKDWFEFAFDYNKFTAKAKDTRTKWTLCTYGTRIRTFRNPARLNQWDNEEVVLTDEFKKAFANADVNIHGNLKEAICSLQDKKYLEPLMHLMKLLLQMRNSITNSEVDYLLSPVADENGTFYDSRVCSASLPKDADANGAYNIARKGLWAIHQIQETPAGDRPNLAIKNKDWLNFAQQKPYMNE

SEQ ID NO: 203 - protein, 1236 aa, synthetic construct
MKQLSELTGLYSLSKTLRFELKPIGKTLEHIESKGLIAQDEQRAEEYKKVKDIIDRYHKKFITMCLSDFKFEQNSLETYVALAEDTNRDEKAFDEIKKTLRKQIVETFKKGGSYSDLFKKELILKHLPEFVSDEEEKKMIANFSKFTTYFNGFNRNRKNMYSDEDKSTAIAYRLIHENLPMFVNNMKSFSRIADSDVHQHFTEIESSFSEYLGVEHLEEMFQLDYFSETLTQEQIAVYNHIIGGRALEDGTKLKGINEYVNLYNQQHKDSRLPLLKPLYKMILSDRVALPWFPSEEFASDEEMIRAINETYNTLKEVLSIDGDSSLRSLLLNISSYDIKHIYIANDLGLTDISQQMFGQYDVYTSAIKQELREKNAPTAKERRNPELYDERINKLFKSARSFSIEHLNSLVDSEHTIQNYYKHLGAYDRNGEQRVNLFTQIEMAYVAAKEILSGKHGNINQSDAETAIIKGLLDAFKSMQRFIKPLLGNGEEADKDNEFDAKLREAWNTLDIVTPLYNKVRNWLTRKPYSTEKIKLNFENAQLLGGWDVNREPDCTSILLRKNGLYYLAIMNKKYNHAFDIDNLPNDGKCYEKIDYKLLPGANKMLPKVFFSKSRIKEFAPSEIIVNAYKKGSHKKGANFSISDCRRLIDFFKASISKHEDWKNFPFSFSSTETYEDISGFYREVEQQGYMLGFRNVSESFIKQLVDEGKLYLFQIWNKDFSDFSKGTPNMHTLYWKMLFDEHNLANVVYKLNGQAEVFYRKSSLDVANTTIHKAHQPIMNKNQENKKRQSTFDYDIIKNRRYTVDKFQFHVPISINFKATGRDNVNDNVLDIIRNGGIKHIIGIDRGERHLLYISLIDLKGNIVKQMTLNDIVNEYNGNTYATNYKNLLAEREGNRTEARKNWKKIENIKDIKKGYLSQVVHIISKMMVEYNAIVVLEDLNMGFMRGRQKIERSVYEQFEKMLIDKLNYYVDKQKNSDEAGGLLHALQLTSKFESFKKLGKQSGCLFYIPAWNTSKIDPVTGFVNLFDTRYTNADQARKFFSQFDSIRYNADKDWFEFAFDYNKFTAKAKDTRTKWTLCTYGTRIRTFRNPARLNQWDNEEVVLTDEFKKAFANADVNIHGNLKEAICSLQDKKYLEPLMHLMKLLLQMRNSITNSEVDYLLSPVADENGTFYDSRVCSASLPKDADANGAYNIARKGLWAIHQIQETPAGDRPNLAIKNKDWLNFAQQKPYMNE

SEQ ID NO: 204 - protein, 1236 aa, synthetic construct
MKQLSELTGLYSLSKTLRFELKPIGKTLEHIESKGLIAQDEQRAEEYKKVKDIIDRYHKKFITMCLSDFKFEQNSLETYVALAEDTNRDEKAFDEIKKTLRKQIVETFKKGGSYSDLFKKELILKHLPEFVSDEEEKKMIANFSKFTTYFNGFNENRKNMYSDEDKSTAIAYRLIHENLPMFVNNMKSFSRIADSDVHQHFTEIESSFSEYLGVEHLEEMFQLDYFSETLTQEQIAVYNHIIGGRALEDGTKLKGINEYVNLYNQQHKDSRLPLLKPLYKMILSDRVALPWFPSEEFASDEEMIRAINETYNTLKEVLSIDGDSSLRSLLLNISSYDIKHIYIANDLGLTDISQQMFGQYDVYTSAIKQELREKNAPTAKERRNPELYDERINKLFKSARSFSIEHLNSLVDSEHTIQNYYKHLGAYDRNGEQRVNLFTQIEMAYVAAKEILSGKHGNINQSDAETAIIKGLLDAFKSMQRFIKPLLGNGEEADKDNEFDAKLREAWNTLDIVTPLYNKVRNWLTRKPYSTEKIKLNFENAQLLRGWDVNREPDCTSILLRKNGLYYLAIMNKKYNHAFDIDNLPNDGKCYEKIDYKLLPGANKMLPKVFFSKSRIKEFAPSEIIVNAYKKGSHKKGANFSISDCRRLIDFFKASISKHEDWKNFPFSFSSTETYEDISGFYREVEQQGYMLGFRNVSESFIKQLVDEGKLYLFQIWNKDFSDFSKGTPNMHTLYWKMLFDEHNLANVVYKLNGQAEVFYRKSSLDVANTTIHKAHQPIMNKNQENKKRQSTFDYDIIKNRRYTVDKFQFHVPISINFKATGRDNVNDNVLDIIRNGGIKHIIGIDRGERHLLYISLIDLKGNIVKQMTLNDIVNEYNGNTYATNYKNLLAEREGNRTEARKNWKKIENIKDIKKGYLSQVVHIISKMMVEYNAIVVLEDLNMGFMRGRQKIERSVYEQFEKMLIDKLNYYVDKQKNSDEAGGLLHALQLTSKFESFKKLGKQSGCLFYIPAWNTSKIDPVTGFVNLFDTRYTNADQARKFFSQFDSIRYNADKDWFEFAFDYNKFTAKAKDTRTKWTLCTYGTRIRTFRNPARLNQWDNEEVVLTDEFKKAFANADVNIHGNLKEAICSLQDKKYLEPLMHLMKLLLQMRNSITNSEVDYLLSPVADENGTFYDSRVCSASLPKDADANGAYNIARKGLWAIHQIQETPAGDRPNLAIKNKDWLNFAQQKPYMNE

SEQ ID NO: 205 - protein, 1236 aa, synthetic construct
MKQLSELTGLYSLSKTLRFELKPIGKTLEHIESKGLIAQDEQRAEEYKKVKDIIDRYHKKFITMCLSDFKFEQNSLETYVALAEDTNRDEKAFDEIKKTLRKQIVETFKKGGSYSDLFKKELILKHLPEFVSDEEEKKMIANFSKFTTYFNGFNRNRKNMYSDEDKSTAIAYRLIHENLPMFVNNMKSFSRIADSDVHQHFTEIESSFSEYLGVEHLEEMFQLDYFSETLTQEQIAVYNHIIGGRALEDGTKLKGINEYVNLYNQQHKDSRLPLLKPLYKMILSDRVALPWFPSEEFASDEEMIRAINETYNTLKEVLSIDGDSSLRSLLLNISSYDIKHIYIANDLGLTDISQQMFGQYDVYTSAIKQELREKNAPTAKERRNPELYDERINKLFKSARSFSIEHLNSLVDSEHTIQNYYKHLGAYDRNGEQRVNLFTQIEMAYVAAKEILSGKHGNINQSDAETAIIKGLLDAFKSMQRFIKPLLGNGEEADKDNEFDAKLREAWNTLDIVTPLYNKVRNWLTRKPYSTEKIKLNFENAQLLRGWDVNREPDCTSILLRKNGLYYLAIMNKKYNHAFDIDNLPNDGKCYEKIDYKLLPGANKMLPKVFFSKSRIKEFAPSEIIVNAYKKGSHKKGANFSISDCRRLIDFFKASISKHEDWKNFPFSFSSTETYEDISGFYREVEQQGYMLGFRNVSESFIKQLVDEGKLYLFQIWNKDFSDFSKGTPNMHTLYWKMLFDEHNLANVVYKLNGQAEVFYRKSSLDVANTTIHKAHQPIMNKNQENKKRQSTFDYDIIKNRRYTVDKFQFHVPISINFKATGRDNVNDNVLDIIRNGGIKHIIGIDRGERHLLYISLIDLKGNIVKQMTLNDIVNEYNGNTYATNYKNLLAEREGNRTEARKNWKKIENIKDIKKGYLSQVVHIISKMMVEYNAIVVLEDLNMGFMRGRQKIERSVYEQFEKMLIDKLNYYVDKQKNSDEAGGLLHALQLTSKFESFKKLGKQSGCLFYIPAWNTSKIDPVTGFVNLFDTRYTNADQARKFFSQFDSIRYNADKDWFEFAFDYNKFTAKAKDTRTKWTLCTYGTRIRTFRNPARLNQWDNEEVVLTDEFKKAFANADVNIHGNLKEAICSLQDKKYLEPLMHLMKLLLQMRNSITNSEVDYLLSPVADENGTFYDSRVCSASLPKDADANGAYNIARKGLWAIHQIQETPAGDRPNLAIKNKDWLNFAQQKPYMNE

SEQ ID NO: 206 - protein, 1236 aa, synthetic construct
MKQLSELTGLYSLSKTLRFELKPIGKTLEHIESKGLIAQDEQRAEEYKKVKDIIDRYHKKFITMCLSDFKFEQNSLETYVALAEDTNRDEKAFDEIKKTLRKQIVETFKKGGSYSDLFKKELILKHLPEFVSDEEEKKMIANFSKFTTYFNGFNRNRKNMYSDEDKSTAIAYRLIHENLPMFVNNMKSFSRIADSDVHQHFTEIESSFSEYLGVEHLEEMFQLDYFSETLTQEQIAVYNHIIGGRALEDGTKLKGINEYVALYNQQHKDSRLPLLKPLYKMILSDRVALPWFPSEEFASDEEMIRAINETYNTLKEVLSIDGDSSLRSLLLNISSYDIKHIYIANDLGLTDISQQMFGQYDVYTSAIKQELREKNAPTAKERRNPELYDERINKLFKSARSFSIEHLNSLVDSEHTIQNYYKHLGAYDRNGEQRVNLFTQIEMAYVAAKEILSGKHGNINQSDAETAIIKGLLDAFKSMQRFIKPLLGNGEEADKDNEFDAKLREAWNTLDIVTPLYNKVRNWLTRKPYSTEKIKLNFENAQLLGGWDVNREPDCTSILLRKNGLYYLAIMNKKYNHAFDIDNLPNDGKCYEKIDYKLLPGANKMLPKVFFSKSRIKEFAPSEIIVNAYKKGSHKKGANFSISDCRRLIDFFKASISKHEDWKNFPFSFSSTETYEDISGFYREVEQQGYMLGFRNVSESFIKQLVDEGKLYLFQIWNKDFSDFSKGTPNMHTLYWKMLFDEHNLANVVYKLNGQAEVFYRKSSLDVANTTIHKAHQPIMNKNQENKKRQSTFDYDIIKNRRYTVDKFQFHVPISINFKATGRDNVNDNVLDIIRNGGIKHIIGIDRGERHLLYISLIDLKGNIVKQMTLNDIVNEYNGNTYATNYKNLLAEREGNRTEARKNWKKIENIKDIKKGYLSQVVHIISKMMVEYNAIVVLEDLNMGFMRGRQKIERSVYEQFEKMLIDKLNYYVDKQKNSDEAGGLLHALQLTSKFESFKKLGKQSGCLFYIPAWNTSKIDPVTGFVNLFDTRYTNADQARKFFSQFDSIRYNADKDWFEFAFDYNKFTAKAKDTRTKWTLCTYGTRIRTFRNPARLNQWDNEEVVLTDEFKKAFANADVNIHGNLKEAICSLQDKKYLEPLMHLMKLLLQMRNSITNSEVDYLLSPVADENGTFYDSRVCSASLPKDADANGAYNIARKGLWAIHQIQETPAGDRPNLAIKNKDWLNFAQQKPYMNE

SEQ ID NO: 207 - protein, 1236 aa, synthetic construct
MKQLSELTGLYSLSKTLRFELKPIGKTLEHIESKGLIAQDEQRAEEYKKVKDIIDRYHKKFITMCLSDFKFEQNSLETYVALAEDTNRDEKAFDEIKKTLRKQIVETFKKGGSYSDLFKKELILKHLPEFVSDEEEKKMIANFSKFTTYFNGFNENRKNMYSDEDKSTAIAYRLIHENLPMFVNNMKSFSRIADSDVHQHFTEIESSFSEYLGVEHLEEMFQLDYFSETLTQEQIAVYNHIIGGRALEDGTKLKGINEYVNLYNQQHKDSRLPLLKPLYKMILSDRVALPWFPSEEFASDEEMIRAINETYNTLKEVLSIDGDSSLRSLLLNISSYDIKHIYIANDLGLTDISQQMFGQYDVYTSAIKQELREKNAPTAKERRNPELYDERINKLFKSARSFSIEHLNSLVDSEHTIQNYYKHLGAYDRNGEQRVNLFTQIEMAYVAAKEILSGKHGNINQSDAETAIIKGLLDAFKSMQRFIKPLLGNGEEADKDNEFDAKLREAWNTLDIVTPLYNKVRNWLTRKPYSTEKIKLNFERAQLLGGWDVNKEPDCTSILLRKNGLYYLAIMNKKYNHAFDIDNLPNDGKCYEKIDYKLLPGANKMLPKVFFSKSRIKEFAPSEIIVNAYKKGSHKKGANFSISDCRRLIDFFKASISKHEDWKNFPFSFSSTETYEDISGFYREVEQQGYMLGFRNVSESFIKQLVDEGKLYLFQIWNKDFSDFSKGTPNMHTLYWKMLFDEHNLANVVYKLNGQAEVFYRKSSLDVANTTIHKAHQPIMNKNQENKKRQSTFDYDIIKNRRYTVDKFQFHVPISINFKATGRDNVNDNVLDIIRNGGIKHIIGIDRGERHLLYISLIDLKGNIVKQMTLNDIVNEYNGNTYATNYKNLLAEREGNRTEARKNWKKIENIKDIKKGYLSQVVHIISKMMVEYNAIVVLEDLNMGFMRGRQKIERSVYEQFEKMLIDKLNYYVDKQKNSDEAGGLLHALQLTSKFESFKKLGKQSGCLFYIPAWNTSKIDPVTGFVNLFDTRYTNADQARKFFSQFDSIRYNADKDWFEFAFDYNKFTAKAKDTRTKWTLCTYGTRIRTFRNPARLNQWDNEEVVLTDEFKKAFANADVNIHGNLKEAICSLQDKKYLEPLMHLMKLLLQMRNSITNSEVDYLLSPVADENGTFYDSRVCSASLPKDADANGAYNIARKGLWAIHQIQETPAGDRPNLAIKNKDWLNFAQQKPYMNE

SEQ ID NO: 208 - protein, 1238 aa, synthetic construct
MNTYEELTGLYSLIKTINFALNPIGETLRHIEEKGFIAQDEQRAEDYKKVKDIIDRYHKYFITMCLDGFKFSTDKLMKYVALAENAKRDEDAFDEVKTQLRKEIVAAFEKGETYKDLFKKELILKHLPNFVTDENEKRLVASFNKFTTYFKKFHRCRKNMYSDEAKSTAIAHRLIHENLPMFYDNMKSFKKIAACEDCIDLAEFEAAFSEEFKAFKVEHLADIFQLEYFNNTLTQRKIDFYKLVVGGKVLEDGTKLKGVNEYVNLYNQQHKDARLPLLKPLYKMILSDKVALSWLPEEFKSDEEMVEAIQEMHKNLKGALTDNAENNLRDLLLNMGKYDLAHIYIANDLGLRDISQNIFGQYDVYTSALKQALRNEYAPTTDECQNLELYDERINKLFKSRTSFSIAELNSLADCDHTIENHFKQLGAAERNGKQEIDLFSQIETAYAKASDILSGKHGNIGQSEAETALVKNLLDAYKSLQHFIKPLLGHGDEAEKDNEFDAKLRAAWDALDIINPLYNKVRNWLTRKPYSTEKIKLNFENAQLLDGWDKNKETVNTSVLLRKDGMYYLAIMNKKANDAFACDELPSDGDCYEKIDYKQISNARMDLPRVLLPPKNIDKYAPSEEIKDIYSKRTYRKGKNFNLEHCRRLIDFFKRSITKHKDWKNFQFIFSPTEKYECISSFYNEVEQQAYMLDFRKVSVAYINQLVESGNLYLFQIWNKDFSKFSKGTPNMHTLYWKQLFDERNLADVVYKLNWQAEVFYRKRSLKLEETTVHHAHQPIPNKNKQNEKRQSQFNYDIVKNRRFTVDKFQFHVSITLNFKATGGGDINDKVLEIIRNNGIKHVIGIDRGERHLLYLSLIDLKGNIIKQMTLNDIVNEYRGHTYTTNYKDLLAEREDNRTEARRNWKRIDNIKEIKQGYLAQVVHIISKMMVEYKAIVILEDLNMGFMRRWQKIERSVYEQFEKSLIEKLNYYVDKQKDENEVGGLLHAFQLTSKFKSFKELGKQSGCLFYVPAWNTSKIDPVTGFVNLFDTKNVKGEKVRAFFSNFDAIRYNAERDWFEFAFNYSNFTNKARDTREEWTLCTHGTSIRTFRNPSKINHRDSEEVVLTDEFKKVFEKAGIDICGNLKNAICALKEKAHLEKLMQLMKLLLQMRNSKPNTEVDYMISPVADEQGNFYDSRCGNSALPDNADANGAYNIARKGLWVIKQIRETPAGKRPDWAIKNKEWLKFAQQKPYLDD

SEQ ID NO: 209 - protein, 1238 aa, synthetic construct
MNTYEELTGLYSLIKTINFALNPIGETLRHIEEKGFIAQDEQRAEDYKKVKDIIDRYHKYFITMCLDGFKFSTDKLMKYVALAENAKRDEDAFDEVKTQLRKEIVAAFEKGETYKDLFKKELILKHLPNFVTDENEKRLVASFNKFTTYFKKFHKCRKNMYSDEAKSTAIAHRLIHENLPMFYDNMKSFKKIAACEDCIDLAEFEAAFSEEFKAFKVEHLADIFQLEYFNNTLTQRKIDFYKLVVGGKVLEDGTKLKGVNEYVNLYNQQHKDARLPLLKPLYKMILSDKVALSWLPEEFKSDEEMVEAIQEMHKNLKGALTDNAENNLRDLLLNMGKYDLAHIYIANDLGLRDISQNIFGQYDVYTSALKQALRNEYAPTTDECQNLELYDERINKLFKSRTSFSIAELNSLADCDHTIENHFKQLGAAERNGKQEIDLFSQIETAYAKASDILSGKHGNIGQSEAETALVKNLLDAYKSLQHFIKPLLGHGDEAEKDNEFDAKLRAAWDALDIINPLYNKVRNWLTRKPYSTEKIKLNFENAQLLRGWDKNKETVNTSVLLRKDGMYYLAIMNKKANDAFACDELPSDGDCYEKIDYKQISNARMDLPRVLLPPKNIDKYAPSEEIKDIYSKRTYRKGKNFNLEHCRRLIDFFKRSITKHKDWKNFQFIFSPTEKYECISSFYNEVEQQAYMLDFRKVSVAYINQLVESGNLYLFQIWNKDFSKFSKGTPNMHTLYWKQLFDERNLADVVYKLNWQAEVFYRKRSLKLEETTVHHAHQPIPNKNKQNEKRQSQFNYDIVKNRRFTVDKFQFHVSITLNFKATGGGDINDKVLEIIRNNGIKHVIGIDRGERHLLYLSLIDLKGNIIKQMTLNDIVNEYRGHTYTTNYKDLLAEREDNRTEARRNWKRIDNIKEIKQGYLAQVVHIISKMMVEYKAIVILEDLNMGFMRRWQKIERSVYEQFEKSLIEKLNYYVDKQKDENEVGGLLHAFQLTSKFKSFKELGKQSGCLFYVPAWNTSKIDPVTGFVNLFDTKNVKGEKVRAFFSNFDAIRYNAERDWFEFAFNYSNFTNKARDTREEWTLCTHGTSIRTFRNPSKINHRDSEEVVLTDEFKKVFEKAGIDICGNLKNAICALKEKAHLEKLMQLMKLLLQMRNSKPNTEVDYMISPVADEQGNFYDSRCGNSALPDNADANGAYNIARKGLWVIKQIRETPAGKRPDWAIKNKEWLKFAQQKPYLDD

SEQ ID NO: 210 - protein, 1238 aa, synthetic construct
MNTYEELTGLYSLIKTINFALNPIGETLRHIEEKGFIAQDEQRAEDYKKVKDIIDRYHKYFITMCLDGFKFSTDKLMKYVALAENAKRDEDAFDEVKTQLRKEIVAAFEKGETYKDLFKKELILKHLPNFVTDENEKRLVASFNKFTTYFKKFHKCRKNMYSDEAKSTAIAHRLIHENLPMFYDNMKSFKKIAACEDCIDLAEFEAAFSEEFKAFKVEHLADIFQLEYFNNTLTQRKIDFYKLVVGGKVLEDGTKLKGVNEYVNLYNQQHKDARLPLLKPLYKMILSDKVALSWLPEEFKSDEEMVEAIQEMHKNLKGALTDNAENNLRDLLLNMGKYDLAHIYIANDLGLRDISQNIFGQYDVYTSALKQALRNEYAPTTDECQNLELYDERINKLFKSRTSFSIAELNSLADCDHTIENHFKQLGAAERNGKQEIDLFSQIETAYAKASDILSGKHGNIGQSEAETALVKNLLDAYKSLQHFIKPLLGHGDEAEKDNEFDAKLRAAWDALDIINPLYNKVRNWLTRKPYSTEKIKLNFENAQLLDGWDKNRETVNTSVLLRKDGMYYLAIMNKKANDAFACDELPSDGDCYEKIDYKQISNARMDLPRVLLPPKNIDKYAPSEEIKDIYSKRTYRKGKNFNLEHCRRLIDFFKRSITKHKDWKNFQFIFSPTEKYECISSFYNEVEQQAYMLDFRKVSVAYINQLVESGNLYLFQIWNKDFSKFSKGTPNMHTLYWKQLFDERNLADVVYKLNWQAEVFYRKRSLKLEETTVHHAHQPIPNKNKQNEKRQSQFNYDIVKNRRFTVDKFQFHVSITLNFKATGGGDINDKVLEIIRNNGIKHVIGIDRGERHLLYLSLIDLKGNIIKQMTLNDIVNEYRGHTYTTNYKDLLAEREDNRTEARRNWKRIDNIKEIKQGYLAQVVHIISKMMVEYKAIVILEDLNMGFMRRWQKIERSVYEQFEKSLIEKLNYYVDKQKDENEVGGLLHAFQLTSKFKSFKELGKQSGCLFYVPAWNTSKIDPVTGFVNLFDTKNVKGEKVRAFFSNFDAIRYNAERDWFEFAFNYSNFTNKARDTREEWTLCTHGTSIRTFRNPSKINHRDSEEVVLTDEFKKVFEKAGIDICGNLKNAICALKEKAHLEKLMQLMKLLLQMRNSKPNTEVDYMISPVADEQGNFYDSRCGNSALPDNADANGAYNIARKGLWVIKQIRETPAGKRPDWAIKNKEWLKFAQQKPYLDD

SEQ ID NO: 211 - protein, 1238 aa, synthetic construct
MNTYEELTGLYSLIKTINFALNPIGETLRHIEEKGFIAQDEQRAEDYKKVKDIIDRYHKYFITMCLDGFKFSTDKLMKYVALAENAKRDEDAFDEVKTQLRKEIVAAFEKGETYKDLFKKELILKHLPNFVTDENEKRLVASFNKFTTYFKKFHRCRKNMYSDEAKSTAIAHRLIHENLPMFYDNMKSFKKIAACEDCIDLAEFEAAFSEEFKAFKVEHLADIFQLEYFNNTLTQRKIDFYKLVVGGKVLEDGTKLKGVNEYVNLYNQQHKDARLPLLKPLYKMILSDKVALSWLPEEFKSDEEMVEAIQEMHKNLKGALTDNAENNLRDLLLNMGKYDLAHIYIANDLGLRDISQNIFGQYDVYTSALKQALRNEYAPTTDECQNLELYDERINKLFKSRTSFSIAELNSLADCDHTIENHFKQLGAAERNGKQEIDLFSQIETAYAKASDILSGKHGNIGQSEAETALVKNLLDAYKSLQHFIKPLLGHGDEAEKDNEFDAKLRAAWDALDIINPLYNKVRNWLTRKPYSTEKIKLNFENAQLLRGWDKNKETVNTSVLLRKDGMYYLAIMNKKANDAFACDELPSDGDCYEKIDYKQISNARMDLPRVLLPPKNIDKYAPSEEIKDIYSKRTYRKGKNFNLEHCRRLIDFFKRSITKHKDWKNFQFIFSPTEKYECISSFYNEVEQQAYMLDFRKVSVAYINQLVESGNLYLFQIWNKDFSKFSKGTPNMHTLYWKQLFDERNLADVVYKLNWQAEVFYRKRSLKLEETTVHHAHQPIPNKNKQNEKRQSQFNYDIVKNRRFTVDKFQFHVSITLNFKATGGGDINDKVLEIIRNNGIKHVIGIDRGERHLLYLSLIDLKGNIIKQMTLNDIVNEYRGHTYTTNYKDLLAEREDNRTEARRNWKRIDNIKEIKQGYLAQVVHIISKMMVEYKAIVILEDLNMGFMRRWQKIERSVYEQFEKSLIEKLNYYVDKQKDENEVGGLLHAFQLTSKFKSFKELGKQSGCLFYVPAWNTSKIDPVTGFVNLFDTKNVKGEKVRAFFSNFDAIRYNAERDWFEFAFNYSNFTNKARDTREEWTLCTHGTSIRTFRNPSKINHRDSEEVVLTDEFKKVFEKAGIDICGNLKNAICALKEKAHLEKLMQLMKLLLQMRNSKPNTEVDYMISPVADEQGNFYDSRCGNSALPDNADANGAYNIARKGLWVIKQIRETPAGKRPDWAIKNKEWLKFAQQKPYLDD

SEQ ID NO: 212 - protein, 1238 aa, synthetic construct
MNTYEELTGLYSLIKTINFALNPIGETLRHIEEKGFIAQDEQRAEDYKKVKDIIDRYHKYFITMCLDGFKFSTDKLMKYVALAENAKRDEDAFDEVKTQLRKEIVAAFEKGETYKDLFKKELILKHLPNFVTDENEKRLVASFNKFTTYFKKFHRCRKNMYSDEAKSTAIAHRLIHENLPMFYDNMKSFKKIAACEDCIDLAEFEAAFSEEFKAFKVEHLADIFQLEYFNNTLTQRKIDFYKLVVGGKVLEDGTKLKGVNEYVNLYNQQHKDARLPLLKPLYKMILSDKVALSWLPEEFKSDEEMVEAIQEMHKNLKGALTDNAENNLRDLLLNMGKYDLAHIYIANDLGLRDISQNIFGQYDVYTSALKQALRNEYAPTTDECQNLELYDERINKLFKSRTSFSIAELNSLADCDHTIENHFKQLGAAERNGKQEIDLFSQIETAYAKASDILSGKHGNIGQSEAETALVKNLLDAYKSLQHFIKPLLGHGDEAEKDNEFDAKLRAAWDALDIINPLYNKVRNWLTRKPYSTEKIKLNFENAQLLDGWDKNRETVNTSVLLRKDGMYYLAIMNKKANDAFACDELPSDGDCYEKIDYKQISNARMDLPRVLLPPKNIDKYAPSEEIKDIYSKRTYRKGKNFNLEHCRRLIDFFKRSITKHKDWKNFQFIFSPTEKYECISSFYNEVEQQAYMLDFRKVSVAYINQLVESGNLYLFQIWNKDFSKFSKGTPNMHTLYWKQLFDERNLADVVYKLNWQAEVFYRKRSLKLEETTVHHAHQPIPNKNKQNEKRQSQFNYDIVKNRRFTVDKFQFHVSITLNFKATGGGDINDKVLEIIRNNGIKHVIGIDRGERHLLYLSLIDLKGNIIKQMTLNDIVNEYRGHTYTTNYKDLLAEREDNRTEARRNWKRIDNIKEIKQGYLAQVVHIISKMMVEYKAIVILEDLNMGFMRRWQKIERSVYEQFEKSLIEKLNYYVDKQKDENEVGGLLHAFQLTSKFKSFKELGKQSGCLFYVPAWNTSKIDPVTGFVNLFDTKNVKGEKVRAFFSNFDAIRYNAERDWFEFAFNYSNFTNKARDTREEWTLCTHGTSIRTFRNPSKINHRDSEEVVLTDEFKKVFEKAGIDICGNLKNAICALKEKAHLEKLMQLMKLLLQMRNSKPNTEVDYMISPVADEQGNFYDSRCGNSALPDNADANGAYNIARKGLWVIKQIRETPAGKRPDWAIKNKEWLKFAQQKPYLDD

SEQ ID NO: 213 - protein, 1238 aa, synthetic construct
MNTYEELTGLYSLIKTINFALNPIGETLRHIEEKGFIAQDEQRAEDYKKVKDIIDRYHKYFITMCLDGFKFSTDKLMKYVALAENAKRDEDAFDEVKTQLRKEIVAAFEKGETYKDLFKKELILKHLPNFVTDENEKRLVASFNKFTTYFKKFHKCRKNMYSDEAKSTAIAHRLIHENLPMFYDNMKSFKKIAACEDCIDLAEFEAAFSEEFKAFKVEHLADIFQLEYFNNTLTQRKIDFYKLVVGGKVLEDGTKLKGVNEYVNLYNQQHKDARLPLLKPLYKMILSDKVALSWLPEEFKSDEEMVEAIQEMHKNLKGALTDNAENNLRDLLLNMGKYDLAHIYIANDLGLRDISQNIFGQYDVYTSALKQALRNEYAPTTDECQNLELYDERINKLFKSRTSFSIAELNSLADCDHTIENHFKQLGAAERNGKQEIDLFSQIETAYAKASDILSGKHGNIGQSEAETALVKNLLDAYKSLQHFIKPLLGHGDEAEKDNEFDAKLRAAWDALDIINPLYNKVRNWLTRKPYSTEKIKLNFENAQLLRGWDKNRETVNTSVLLRKDGMYYLAIMNKKANDAFACDELPSDGDCYEKIDYKQISNARMDLPRVLLPPKNIDKYAPSEEIKDIYSKRTYRKGKNFNLEHCRRLIDFFKRSITKHKDWKNFQFIFSPTEKYECISSFYNEVEQQAYMLDFRKVSVAYINQLVESGNLYLFQIWNKDFSKFSKGTPNMHTLYWKQLFDERNLADVVYKLNWQAEVFYRKRSLKLEETTVHHAHQPIPNKNKQNEKRQSQFNYDIVKNRRFTVDKFQFHVSITLNFKATGGGDINDKVLEIIRNNGIKHVIGIDRGERHLLYLSLIDLKGNIIKQMTLNDIVNEYRGHTYTTNYKDLLAEREDNRTEARRNWKRIDNIKEIKQGYLAQVVHIISKMMVEYKAIVILEDLNMGFMRRWQKIERSVYEQFEKSLIEKLNYYVDKQKDENEVGGLLHAFQLTSKFKSFKELGKQSGCLFYVPAWNTSKIDPVTGFVNLFDTKNVKGEKVRAFFSNFDAIRYNAERDWFEFAFNYSNFTNKARDTREEWTLCTHGTSIRTFRNPSKINHRDSEEVVLTDEFKKVFEKAGIDICGNLKNAICALKEKAHLEKLMQLMKLLLQMRNSKPNTEVDYMISPVADEQGNFYDSRCGNSALPDNADANGAYNIARKGLWVIKQIRETPAGKRPDWAIKNKEWLKFAQQKPYLDD

SEQ ID NO: 214 - protein, 1238 aa, synthetic construct
MNTYEELTGLYSLIKTINFALNPIGETLRHIEEKGFIAQDEQRAEDYKKVKDIIDRYHKYFITMCLDGFKFSTDKLMKYVALAENAKRDEDAFDEVKTQLRKEIVAAFEKGETYKDLFKKELILKHLPNFVTDENEKRLVASFNKFTTYFKKFHRCRKNMYSDEAKSTAIAHRLIHENLPMFYDNMKSFKKIAACEDCIDLAEFEAAFSEEFKAFKVEHLADIFQLEYFNNTLTQRKIDFYKLVVGGKVLEDGTKLKGVNEYVNLYNQQHKDARLPLLKPLYKMILSDKVALSWLPEEFKSDEEMVEAIQEMHKNLKGALTDNAENNLRDLLLNMGKYDLAHIYIANDLGLRDISQNIFGQYDVYTSALKQALRNEYAPTTDECQNLELYDERINKLFKSRTSFSIAELNSLADCDHTIENHFKQLGAAERNGKQEIDLFSQIETAYAKASDILSGKHGNIGQSEAETALVKNLLDAYKSLQHFIKPLLGHGDEAEKDNEFDAKLRAAWDALDIINPLYNKVRNWLTRKPYSTEKIKLNFENAQLLRGWDKNRETVNTSVLLRKDGMYYLAIMNKKANDAFACDELPSDGDCYEKIDYKQISNARMDLPRVLLPPKNIDKYAPSEEIKDIYSKRTYRKGKNFNLEHCRRLIDFFKRSITKHKDWKNFQFIFSPTEKYECISSFYNEVEQQAYMLDFRKVSVAYINQLVESGNLYLFQIWNKDFSKFSKGTPNMHTLYWKQLFDERNLADVVYKLNWQAEVFYRKRSLKLEETTVHHAHQPIPNKNKQNEKRQSQFNYDIVKNRRFTVDKFQFHVSITLNFKATGGGDINDKVLEIIRNNGIKHVIGIDRGERHLLYLSLIDLKGNIIKQMTLNDIVNEYRGHTYTTNYKDLLAEREDNRTEARRNWKRIDNIKEIKQGYLAQVVHIISKMMVEYKAIVILEDLNMGFMRRWQKIERSVYEQFEKSLIEKLNYYVDKQKDENEVGGLLHAFQLTSKFKSFKELGKQSGCLFYVPAWNTSKIDPVTGFVNLFDTKNVKGEKVRAFFSNFDAIRYNAERDWFEFAFNYSNFTNKARDTREEWTLCTHGTSIRTFRNPSKINHRDSEEVVLTDEFKKVFEKAGIDICGNLKNAICALKEKAHLEKLMQLMKLLLQMRNSKPNTEVDYMISPVADEQGNFYDSRCGNSALPDNADANGAYNIARKGLWVIKQIRETPAGKRPDWAIKNKEWLKFAQQKPYLDD

SEQ ID NO: 215 - protein, 1238 aa, synthetic construct
MNTYEELTGLYSLIKTINFALNPIGETLRHIEEKGFIAQDEQRAEDYKKVKDIIDRYHKYFITMCLDGFKFSTDKLMKYVALAENAKRDEDAFDEVKTQLRKEIVAAFEKGETYKDLFKKELILKHLPNFVTDENEKRLVASFNKFTTYFKKFHRCRKNMYSDEAKSTAIAHRLIHENLPMFYDNMKSFKKIAACEDCIDLAEFEAAFSEEFKAFKVEHLADIFQLEYFNNTLTQRKIDFYKLVVGGKVLEDGTKLKGVNEYVALYNQQHKDARLPLLKPLYKMILSDKVALSWLPEEFKSDEEMVEAIQEMHKNLKGALTDNAENNLRDLLLNMGKYDLAHIYIANDLGLRDISQNIFGQYDVYTSALKQALRNEYAPTTDECQNLELYDERINKLFKSRTSFSIAELNSLADCDHTIENHFKQLGAAERNGKQEIDLFSQIETAYAKASDILSGKHGNIGQSEAETALVKNLLDAYKSLQHFIKPLLGHGDEAEKDNEFDAKLRAAWDALDIINPLYNKVRNWLTRKPYSTEKIKLNFENAQLLDGWDKNRETVNTSVLLRKDGMYYLAIMNKKANDAFACDELPSDGDCYEKIDYKQISNARMDLPRVLLPPKNIDKYAPSEEIKDIYSKRTYRKGKNFNLEHCRRLIDFFKRSITKHKDWKNFQFIFSPTEKYECISSFYNEVEQQAYMLDFRKVSVAYINQLVESGNLYLFQIWNKDFSKFSKGTPNMHTLYWKQLFDERNLADVVYKLNWQAEVFYRKRSLKLEETTVHHAHQPIPNKNKQNEKRQSQFNYDIVKNRRFTVDKFQFHVSITLNFKATGGGDINDKVLEIIRNNGIKHVIGIDRGERHLLYLSLIDLKGNIIKQMTLNDIVNEYRGHTYTTNYKDLLAEREDNRTEARRNWKRIDNIKEIKQGYLAQVVHIISKMMVEYKAIVILEDLNMGFMRRWQKIERSVYEQFEKSLIEKLNYYVDKQKDENEVGGLLHAFQLTSKFKSFKELGKQSGCLFYVPAWNTSKIDPVTGFVNLFDTKNVKGEKVRAFFSNFDAIRYNAERDWFEFAFNYSNFTNKARDTREEWTLCTHGTSIRTFRNPSKINHRDSEEVVLTDEFKKVFEKAGIDICGNLKNAICALKEKAHLEKLMQLMKLLLQMRNSKPNTEVDYMISPVADEQGNFYDSRCGNSALPDNADANGAYNIARKGLWVIKQIRETPAGKRPDWAIKNKEWLKFAQQKPYLDD

SEQ ID NO: 216 - protein, 1238 aa, synthetic construct
MNTYEELTGLYSLIKTINFALNPIGETLRHIEEKGFIAQDEQRAEDYKKVKDIIDRYHKYFITMCLDGFKFSTDKLMKYVALAENAKRDEDAFDEVKTQLRKEIVAAFEKGETYKDLFKKELILKHLPNFVTDENEKRLVASFNKFTTYFKKFHKCRKNMYSDEAKSTAIAHRLIHENLPMFYDNMKSFKKIAACEDCIDLAEFEAAFSEEFKAFKVEHLADIFQLEYFNNTLTQRKIDFYKLVVGGKVLEDGTKLKGVNEYVNLYNQQHKDARLPLLKPLYKMILSDKVALSWLPEEFKSDEEMVEAIQEMHKNLKGALTDNAENNLRDLLLNMGKYDLAHIYIANDLGLRDISQNIFGQYDVYTSALKQALRNEYAPTTDECQNLELYDERINKLFKSRTSFSIAELNSLADCDHTIENHFKQLGAAERNGKQEIDLFSQIETAYAKASDILSGKHGNIGQSEAETALVKNLLDAYKSLQHFIKPLLGHGDEAEKDNEFDAKLRAAWDALDIINPLYNKVRNWLTRKPYSTEKIKLNFERAQLLDGWDKNKETVNTSVLLRKDGMYYLAIMNKKANDAFACDELPSDGDCYEKIDYKQISNARMDLPRVLLPPKNIDKYAPSEEIKDIYSKRTYRKGKNFNLEHCRRLIDFFKRSITKHKDWKNFQFIFSPTEKYECISSFYNEVEQQAYMLDFRKVSVAYINQLVESGNLYLFQIWNKDFSKFSKGTPNMHTLYWKQLFDERNLADVVYKLNWQAEVFYRKRSLKLEETTVHHAHQPIPNKNKQNEKRQSQFNYDIVKNRRFTVDKFQFHVSITLNFKATGGGDINDKVLEIIRNNGIKHVIGIDRGERHLLYLSLIDLKGNIIKQMTLNDIVNEYRGHTYTTNYKDLLAEREDNRTEARRNWKRIDNIKEIKQGYLAQVVHIISKMMVEYKAIVILEDLNMGFMRRWQKIERSVYEQFEKSLIEKLNYYVDKQKDENEVGGLLHAFQLTSKFKSFKELGKQSGCLFYVPAWNTSKIDPVTGFVNLFDTKNVKGEKVRAFFSNFDAIRYNAERDWFEFAFNYSNFTNKARDTREEWTLCTHGTSIRTFRNPSKINHRDSEEVVLTDEFKKVFEKAGIDICGNLKNAICALKEKAHLEKLMQLMKLLLQMRNSKPNTEVDYMISPVADEQGNFYDSRCGNSALPDNADANGAYNIARKGLWVIKQIRETPAGKRPDWAIKNKEWLKFAQQKPYLDD

SEQ ID NO: 217 - protein, 1245 aa, synthetic construct
MTLIIKYKESKNKREVYMSNLYSNLHNLYQVQKTLRFELKPQGKTKENMEKVGILEADEHRAEIYSKVKKYCDEYHKLFIDKSLSNIELNGIDRYYELYSINNRDDKQKEELDQLEASLRKQISDAFKKSAEYKGLFQKDIITSYLVTMYKENQEKMQDIGEFNRFTTYFTGYNRNRENMYSEEDKSTAISYRLINENLPTFIDNIKIYKKIVSLMPEDIEKIYKDLEEYIQVDSIDEIFNISYYNDVLTQRGIECYNILISGRTKNDGDKIKGLNEYINEFNQTHNEKIPKLQELYKQILSDAESASFKIDVIKNDKELMNLIEVYYANILPILNKIEDLFTRISNYNLELILVNNDGTLSTLSNMVFNEWSYIKGAISEKYDEEYSGKEKYGTEKYAQKKQEYLKKQKIYSLKFLNDCIGNNAICEYLKNYIIQNKNIETIKEDYNEVQNIKVEDDTKELIKDEKSIEKIKKFLDDVKSLQEFVKLVIPKDRTVEKDAKFYSELTPYYEKIKEIIPLYNKVRNYVTQKPYSTEKIKLNFECPTLLNGWDANKEEANLGVILLKEGKYYLGIINPYCKKIFEVDEKDSNEQNNYKKMEYKLLPGPNKMLPKVFFSNSRIEEFNPSKELQEKYNKGYHKKGKDFDINFCHELIDFYKQSLNKHEDWKKFNFKFKDTSEYNDISEFYREVEKQGYKIEYTEYSEKYINELVDRGELYLFQIYNKDFSEYSKGKENLHTLYWKAVFDPDNIMNPVYKLNGNAEVFYRKKSLEMKVTHPANQPIANKNISTIEAGRSTSTFKYDLIKDKRYTMDKFQFHVPITMNFKSERLLNINQIVNKYLKYNDDIHVIGIDRGERNLLYVCVIDKNEKIVYQKSLNEIVSEYNNNRYTTDYHGLLDRKEKEREIAREDWKNIENIKELKEGYMSQVIHILVELMKKYNAIIVIEDLNKGFKNSRIKVEKQVYQKFEKMFIDKLNYLVFKDEDKMDEGGVLNAYQLTNKFESFTKLGKQSGILYYIPAWCTSKIDPTTGFINRFYLKYENFDKSKEFVNRIDDIRYNEKENLFEFDIDYSKFTDRLNDTKNKWTLCSYGERILTQKNANGEWFDRRIQLSIEFKKLFEKYGINLNNIKDSILKLDKDNLEFYKGNGESLGFIQLFKLMVQMRNSLTGKEEDNLISPVKNQHGKFFNTSEKIEGLPIDADANGAYNIARKGFMLVEQMKNVEDEKLNKIKYNITEKEWLNYVQNRGM

SEQ ID NO: 218 - protein, 1245 aa, synthetic construct
MTLIIKYKESKNKREVYMSNLYSNLHNLYQVQKTLRFELKPQGKTKENMEKVGILEADEHRAEIYSKVKKYCDEYHKLFIDKSLSNIELNGIDRYYELYSINNRDDKQKEELDQLEASLRKQISDAFKKSAEYKGLFQKDIITSYLVTMYKENQEKMQDIGEFNRFTTYFTGYNKNRENMYSEEDKSTAISYRLINENLPTFIDNIKIYKKIVSLMPEDIEKIYKDLEEYIQVDSIDEIFNISYYNDVLTQRGIECYNILISGRTKNDGDKIKGLNEYINEFNQTHNEKIPKLQELYKQILSDAESASFKIDVIKNDKELMNLIEVYYANILPILNKIEDLFTRISNYNLELILVNNDGTLSTLSNMVFNEWSYIKGAISEKYDEEYSGKEKYGTEKYAQKKQEYLKKQKIYSLKFLNDCIGNNAICEYLKNYIIQNKNIETIKEDYNEVQNIKVEDDTKELIKDEKSIEKIKKFLDDVKSLQEFVKLVIPKDRTVEKDAKFYSELTPYYEKIKEIIPLYNKVRNYVTQKPYSTEKIKLNFECPTLLRGWDANKEEANLGVILLKEGKYYLGIINPYCKKIFEVDEKDSNEQNNYKKMEYKLLPGPNKMLPKVFFSNSRIEEFNPSKELQEKYNKGYHKKGKDFDINFCHELIDFYKQSLNKHEDWKKFNFKFKDTSEYNDISEFYREVEKQGYKIEYTEYSEKYINELVDRGELYLFQIYNKDFSEYSKGKENLHTLYWKAVFDPDNIMNPVYKLNGNAEVFYRKKSLEMKVTHPANQPIANKNISTIEAGRSTSTFKYDLIKDKRYTMDKFQFHVPITMNFKSERLLNINQIVNKYLKYNDDIHVIGIDRGERNLLYVCVIDKNEKIVYQKSLNEIVSEYNNNRYTTDYHGLLDRKEKEREIAREDWKNIENIKELKEGYMSQVIHILVELMKKYNAIIVIEDLNKGFKNSRIKVEKQVYQKFEKMFIDKLNYLVFKDEDKMDEGGVLNAYQLTNKFESFTKLGKQSGILYYIPAWCTSKIDPTTGFINRFYLKYENFDKSKEFVNRIDDIRYNEKENLFEFDIDYSKFTDRLNDTKNKWTLCSYGERILTQKNANGEWFDRRIQLSIEFKKLFEKYGINLNNIKDSILKLDKDNLEFYKGNGESLGFIQLFKLMVQMRNSLTGKEEDNLISPVKNQHGKFFNTSEKIEGLPIDADANGAYNIARKGFMLVEQMKNVEDEKLNKIKYNITEKEWLNYVQNRGM

SEQ ID NO: 219 - protein, 1245 aa, synthetic construct
MTLIIKYKESKNKREVYMSNLYSNLHNLYQVQKTLRFELKPQGKTKENMEKVGILEADEHRAEIYSKVKKYCDEYHKLFIDKSLSNIELNGIDRYYELYSINNRDDKQKEELDQLEASLRKQISDAFKKSAEYKGLFQKDIITSYLVTMYKENQEKMQDIGEFNRFTTYFTGYNKNRENMYSEEDKSTAISYRLINENLPTFIDNIKIYKKIVSLMPEDIEKIYKDLEEYIQVDSIDEIFNISYYNDVLTQRGIECYNILISGRTKNDGDKIKGLNEYINEFNQTHNEKIPKLQELYKQILSDAESASFKIDVIKNDKELMNLIEVYYANILPILNKIEDLFTRISNYNLELILVNNDGTLSTLSNMVFNEWSYIKGAISEKYDEEYSGKEKYGTEKYAQKKQEYLKKQKIYSLKFLNDCIGNNAICEYLKNYIIQNKNIETIKEDYNEVQNIKVEDDTKELIKDEKSIEKIKKFLDDVKSLQEFVKLVIPKDRTVEKDAKFYSELTPYYEKIKEIIPLYNKVRNYVTQKPYSTEKIKLNFECPTLLNGWDANREEANLGVILLKEGKYYLGIINPYCKKIFEVDEKDSNEQNNYKKMEYKLLPGPNKMLPKVFFSNSRIEEFNPSKELQEKYNKGYHKKGKDFDINFCHELIDFYKQSLNKHEDWKKFNFKFKDTSEYNDISEFYREVEKQGYKIEYTEYSEKYINELVDRGELYLFQIYNKDFSEYSKGKENLHTLYWKAVFDPDNIMNPVYKLNGNAEVFYRKKSLEMKVTHPANQPIANKNISTIEAGRSTSTFKYDLIKDKRYTMDKFQFHVPITMNFKSERLLNINQIVNKYLKYNDDIHVIGIDRGERNLLYVCVIDKNEKIVYQKSLNEIVSEYNNNRYTTDYHGLLDRKEKEREIAREDWKNIENIKELKEGYMSQVIHILVELMKKYNAIIVIEDLNKGFKNSRIKVEKQVYQKFEKMFIDKLNYLVFKDEDKMDEGGVLNAYQLTNKFESFTKLGKQSGILYYIPAWCTSKIDPTTGFINRFYLKYENFDKSKEFVNRIDDIRYNEKENLFEFDIDYSKFTDRLNDTKNKWTLCSYGERILTQKNANGEWFDRRIQLSIEFKKLFEKYGINLNNIKDSILKLDKDNLEFYKGNGESLGFIQLFKLMVQMRNSLTGKEEDNLISPVKNQHGKFFNTSEKIEGLPIDADANGAYNIARKGFMLVEQMKNVEDEKLNKIKYNITEKEWLNYVQNRGM

SEQ ID NO: 220 - protein, 1245 aa, synthetic construct
MTLIIKYKESKNKREVYMSNLYSNLHNLYQVQKTLRFELKPQGKTKENMEKVGILEADEHRAEIYSKVKKYCDEYHKLFIDKSLSNIELNGIDRYYELYSINNRDDKQKEELDQLEASLRKQISDAFKKSAEYKGLFQKDIITSYLVTMYKENQEKMQDIGEFNRFTTYFTGYNRNRENMYSEEDKSTAISYRLINENLPTFIDNIKIYKKIVSLMPEDIEKIYKDLEEYIQVDSIDEIFNISYYNDVLTQRGIECYNILISGRTKNDGDKIKGLNEYINEFNQTHNEKIPKLQELYKQILSDAESASFKIDVIKNDKELMNLIEVYYANILPILNKIEDLFTRISNYNLELILVNNDGTLSTLSNMVFNEWSYIKGAISEKYDEEYSGKEKYGTEKYAQKKQEYLKKQKIYSLKFLNDCIGNNAICEYLKNYIIQNKNIETIKEDYNEVQNIKVEDDTKELIKDEKSIEKIKKFLDDVKSLQEFVKLVIPKDRTVEKDAKFYSELTPYYEKIKEIIPLYNKVRNYVTQKPYSTEKIKLNFECPTLLRGWDANKEEANLGVILLKEGKYYLGIINPYCKKIFEVDEKDSNEQNNYKKMEYKLLPGPNKMLPKVFFSNSRIEEFNPSKELQEKYNKGYHKKGKDFDINFCHELIDFYKQSLNKHEDWKKFNFKFKDTSEYNDISEFYREVEKQGYKIEYTEYSEKYINELVDRGELYLFQIYNKDFSEYSKGKENLHTLYWKAVFDPDNIMNPVYKLNGNAEVFYRKKSLEMKVTHPANQPIANKNISTIEAGRSTSTFKYDLIKDKRYTMDKFQFHVPITMNFKSERLLNINQIVNKYLKYNDDIHVIGIDRGERNLLYVCVIDKNEKIVYQKSLNEIVSEYNNNRYTTDYHGLLDRKEKEREIAREDWKNIENIKELKEGYMSQVIHILVELMKKYNAIIVIEDLNKGFKNSRIKVEKQVYQKFEKMFIDKLNYLVFKDEDKMDEGGVLNAYQLTNKFESFTKLGKQSGILYYIPAWCTSKIDPTTGFINRFYLKYENFDKSKEFVNRIDDIRYNEKENLFEFDIDYSKFTDRLNDTKNKWTLCSYGERILTQKNANGEWFDRRIQLSIEFKKLFEKYGINLNNIKDSILKLDKDNLEFYKGNGESLGFIQLFKLMVQMRNSLTGKEEDNLISPVKNQHGKFFNTSEKIEGLPIDADANGAYNIARKGFMLVEQMKNVEDEKLNKIKYNITEKEWLNYVQNRGM

SEQ ID NO: 221 - protein, 1245 aa, synthetic construct
MTLIIKYKESKNKREVYMSNLYSNLHNLYQVQKTLRFELKPQGKTKENMEKVGILEADEHRAEIYSKVKKYCDEYHKLFIDKSLSNIELNGIDRYYELYSINNRDDKQKEELDQLEASLRKQISDAFKKSAEYKGLFQKDIITSYLVTMYKENQEKMQDIGEFNRFTTYFTGYNRNRENMYSEEDKSTAISYRLINENLPTFIDNIKIYKKIVSLMPEDIEKIYKDLEEYIQVDSIDEIFNISYYNDVLTQRGIECYNILISGRTKNDGDKIKGLNEYINEFNQTHNEKIPKLQELYKQILSDAESASFKIDVIKNDKELMNLIEVYYANILPILNKIEDLFTRISNYNLELILVNNDGTLSTLSNMVFNEWSYIKGAISEKYDEEYSGKEKYGTEKYAQKKQEYLKKQKIYSLKFLNDCIGNNAICEYLKNYIIQNKNIETIKEDYNEVQNIKVEDDTKELIKDEKSIEKIKKFLDDVKSLQEFVKLVIPKDRTVEKDAKFYSELTPYYEKIKEIIPLYNKVRNYVTQKPYSTEKIKLNFECPTLLNGWDANREEANLGVILLKEGKYYLGIINPYCKKIFEVDEKDSNEQNNYKKMEYKLLPGPNKMLPKVFFSNSRIEEFNPSKELQEKYNKGYHKKGKDFDINFCHELIDFYKQSLNKHEDWKKFNFKFKDTSEYNDISEFYREVEKQGYKIEYTEYSEKYINELVDRGELYLFQIYNKDFSEYSKGKENLHTLYWKAVFDPDNIMNPVYKLNGNAEVFYRKKSLEMKVTHPANQPIANKNISTIEAGRSTSTFKYDLIKDKRYTMDKFQFHVPITMNFKSERLLNINQIVNKYLKYNDDIHVIGIDRGERNLLYVCVIDKNEKIVYQKSLNEIVSEYNNNRYTTDYHGLLDRKEKEREIAREDWKNIENIKELKEGYMSQVIHILVELMKKYNAIIVIEDLNKGFKNSRIKVEKQVYQKFEKMFIDKLNYLVFKDEDKMDEGGVLNAYQLTNKFESFTKLGKQSGILYYIPAWCTSKIDPTTGFINRFYLKYENFDKSKEFVNRIDDIRYNEKENLFEFDIDYSKFTDRLNDTKNKWTLCSYGERILTQKNANGEWFDRRIQLSIEFKKLFEKYGINLNNIKDSILKLDKDNLEFYKGNGESLGFIQLFKLMVQMRNSLTGKEEDNLISPVKNQHGKFFNTSEKIEGLPIDADANGAYNIARKGFMLVEQMKNVEDEKLNKIKYNITEKEWLNYVQNRGM

SEQ ID NO: 222 - protein, 1245 aa, synthetic construct
MTLIIKYKESKNKREVYMSNLYSNLHNLYQVQKTLRFELKPQGKTKENMEKVGILEADEHRAEIYSKVKKYCDEYHKLFIDKSLSNIELNGIDRYYELYSINNRDDKQKEELDQLEASLRKQISDAFKKSAEYKGLFQKDIITSYLVTMYKENQEKMQDIGEFNRFTTYFTGYNKNRENMYSEEDKSTAISYRLINENLPTFIDNIKIYKKIVSLMPEDIEKIYKDLEEYIQVDSIDEIFNISYYNDVLTQRGIECYNILISGRTKNDGDKIKGLNEYINEFNQTHNEKIPKLQELYKQILSDAESASFKIDVIKNDKELMNLIEVYYANILPILNKIEDLFTRISNYNLELILVNNDGTLSTLSNMVFNEWSYIKGAISEKYDEEYSGKEKYGTEKYAQKKQEYLKKQKIYSLKFLNDCIGNNAICEYLKNYIIQNKNIETIKEDYNEVQNIKVEDDTKELIKDEKSIEKIKKFLDDVKSLQEFVKLVIPKDRTVEKDAKFYSELTPYYEKIKEIIPLYNKVRNYVTQKPYSTEKIKLNFECPTLLRGWDANREEANLGVILLKEGKYYLGIINPYCKKIFEVDEKDSNEQNNYKKMEYKLLPGPNKMLPKVFFSNSRIEEFNPSKELQEKYNKGYHKKGKDFDINFCHELIDFYKQSLNKHEDWKKFNFKFKDTSEYNDISEFYREVEKQGYKIEYTEYSEKYINELVDRGELYLFQIYNKDFSEYSKGKENLHTLYWKAVFDPDNIMNPVYKLNGNAEVFYRKKSLEMKVTHPANQPIANKNISTIEAGRSTSTFKYDLIKDKRYTMDKFQFHVPITMNFKSERLLNINQIVNKYLKYNDDIHVIGIDRGERNLLYVCVIDKNEKIVYQKSLNEIVSEYNNNRYTTDYHGLLDRKEKEREIAREDWKNIENIKELKEGYMSQVIHILVELMKKYNAIIVIEDLNKGFKNSRIKVEKQVYQKFEKMFIDKLNYLVFKDEDKMDEGGVLNAYQLTNKFESFTKLGKQSGILYYIPAWCTSKIDPTTGFINRFYLKYENFDKSKEFVNRIDDIRYNEKENLFEFDIDYSKFTDRLNDTKNKWTLCSYGERILTQKNANGEWFDRRIQLSIEFKKLFEKYGINLNNIKDSILKLDKDNLEFYKGNGESLGFIQLFKLMVQMRNSLTGKEEDNLISPVKNQHGKFFNTSEKIEGLPIDADANGAYNIARKGFMLVEQMKNVEDEKLNKIKYNITEKEWLNYVQNRGM

SEQ ID NO: 223 - protein, 1245 aa, synthetic construct
MTLIIKYKESKNKREVYMSNLYSNLHNLYQVQKTLRFELKPQGKTKENMEKVGILEADEHRAEIYSKVKKYCDEYHKLFIDKSLSNIELNGIDRYYELYSINNRDDKQKEELDQLEASLRKQISDAFKKSAEYKGLFQKDIITSYLVTMYKENQEKMQDIGEFNRFTTYFTGYNRNRENMYSEEDKSTAISYRLINENLPTFIDNIKIYKKIVSLMPEDIEKIYKDLEEYIQVDSIDEIFNISYYNDVLTQRGIECYNILISGRTKNDGDKIKGLNEYINEFNQTHNEKIPKLQELYKQILSDAESASFKIDVIKNDKELMNLIEVYYANILPILNKIEDLFTRISNYNLELILVNNDGTLSTLSNMVFNEWSYIKGAISEKYDEEYSGKEKYGTEKYAQKKQEYLKKQKIYSLKFLNDCIGNNAICEYLKNYIIQNKNIETIKEDYNEVQNIKVEDDTKELIKDEKSIEKIKKFLDDVKSLQEFVKLVIPKDRTVEKDAKFYSELTPYYEKIKEIIPLYNKVRNYVTQKPYSTEKIKLNFECPTLLRGWDANREEANLGVILLKEGKYYLGIINPYCKKIFEVDEKDSNEQNNYKKMEYKLLPGPNKMLPKVFFSNSRIEEFNPSKELQEKYNKGYHKKGKDFDINFCHELIDFYKQSLNKHEDWKKFNFKFKDTSEYNDISEFYREVEKQGYKIEYTEYSEKYINELVDRGELYLFQIYNKDFSEYSKGKENLHTLYWKAVFDPDNIMNPVYKLNGNAEVFYRKKSLEMKVTHPANQPIANKNISTIEAGRSTSTFKYDLIKDKRYTMDKFQFHVPITMNFKSERLLNINQIVNKYLKYNDDIHVIGIDRGERNLLYVCVIDKNEKIVYQKSLNEIVSEYNNNRYTTDYHGLLDRKEKEREIAREDWKNIENIKELKEGYMSQVIHILVELMKKYNAIIVIEDLNKGFKNSRIKVEKQVYQKFEKMFIDKLNYLVFKDEDKMDEGGVLNAYQLTNKFESFTKLGKQSGILYYIPAWCTSKIDPTTGFINRFYLKYENFDKSKEFVNRIDDIRYNEKENLFEFDIDYSKFTDRLNDTKNKWTLCSYGERILTQKNANGEWFDRRIQLSIEFKKLFEKYGINLNNIKDSILKLDKDNLEFYKGNGESLGFIQLFKLMVQMRNSLTGKEEDNLISPVKNQHGKFFNTSEKIEGLPIDADANGAYNIARKGFMLVEQMKNVEDEKLNKIKYNITEKEWLNYVQNRGM

SEQ ID NO: 224 - protein, 1245 aa, synthetic construct
MTLIIKYKESKNKREVYMSNLYSNLHNLYQVQKTLRFELKPQGKTKENMEKVGILEADEHRAEIYSKVKKYCDEYHKLFIDKSLSNIELNGIDRYYELYSINNRDDKQKEELDQLEASLRKQISDAFKKSAEYKGLFQKDIITSYLVTMYKENQEKMQDIGEFNRFTTYFTGYNRNRENMYSEEDKSTAISYRLINENLPTFIDNIKIYKKIVSLMPEDIEKIYKDLEEYIQVDSIDEIFNISYYNDVLTQRGIECYNILISGRTKNDGDKIKGLNEYIAEFNQTHNEKIPKLQELYKQILSDAESASFKIDVIKNDKELMNLIEVYYANILPILNKIEDLFTRISNYNLELILVNNDGTLSTLSNMVFNEWSYIKGAISEKYDEEYSGKEKYGTEKYAQKKQEYLKKQKIYSLKFLNDCIGNNAICEYLKNYIIQNKNIETIKEDYNEVQNIKVEDDTKELIKDEKSIEKIKKFLDDVKSLQEFVKLVIPKDRTVEKDAKFYSELTPYYEKIKEIIPLYNKVRNYVTQKPYSTEKIKLNFECPTLLNGWDANREEANLGVILLKEGKYYLGIINPYCKKIFEVDEKDSNEQNNYKKMEYKLLPGPNKMLPKVFFSNSRIEEFNPSKELQEKYNKGYHKKGKDFDINFCHELIDFYKQSLNKHEDWKKFNFKFKDTSEYNDISEFYREVEKQGYKIEYTEYSEKYINELVDRGELYLFQIYNKDFSEYSKGKENLHTLYWKAVFDPDNIMNPVYKLNGNAEVFYRKKSLEMKVTHPANQPIANKNISTIEAGRSTSTFKYDLIKDKRYTMDKFQFHVPITMNFKSERLLNINQIVNKYLKYNDDIHVIGIDRGERNLLYVCVIDKNEKIVYQKSLNEIVSEYNNNRYTTDYHGLLDRKEKEREIAREDWKNIENIKELKEGYMSQVIHILVELMKKYNAIIVIEDLNKGFKNSRIKVEKQVYQKFEKMFIDKLNYLVFKDEDKMDEGGVLNAYQLTNKFESFTKLGKQSGILYYIPAWCTSKIDPTTGFINRFYLKYENFDKSKEFVNRIDDIRYNEKENLFEFDIDYSKFTDRLNDTKNKWTLCSYGERILTQKNANGEWFDRRIQLSIEFKKLFEKYGINLNNIKDSILKLDKDNLEFYKGNGESLGFIQLFKLMVQMRNSLTGKEEDNLISPVKNQHGKFFNTSEKIEGLPIDADANGAYNIARKGFMLVEQMKNVEDEKLNKIKYNITEKEWLNYVQNRGM

SEQ ID NO: 225 - protein, 1245 aa, synthetic construct
MTLIIKYKESKNKREVYMSNLYSNLHNLYQVQKTLRFELKPQGKTKENMEKVGILEADEHRAEIYSKVKKYCDEYHKLFIDKSLSNIELNGIDRYYELYSINNRDDKQKEELDQLEASLRKQISDAFKKSAEYKGLFQKDIITSYLVTMYKENQEKMQDIGEFNRFTTYFTGYNKNRENMYSEEDKSTAISYRLINENLPTFIDNIKIYKKIVSLMPEDIEKIYKDLEEYIQVDSIDEIFNISYYNDVLTQRGIECYNILISGRTKNDGDKIKGLNEYINEFNQTHNEKIPKLQELYKQILSDAESASFKIDVIKNDKELMNLIEVYYANILPILNKIEDLFTRISNYNLELILVNNDGTLSTLSNMVFNEWSYIKGAISEKYDEEYSGKEKYGTEKYAQKKQEYLKKQKIYSLKFLNDCIGNNAICEYLKNYIIQNKNIETIKEDYNEVQNIKVEDDTKELIKDEKSIEKIKKFLDDVKSLQEFVKLVIPKDRTVEKDAKFYSELTPYYEKIKEIIPLYNKVRNYVTQKPYSTEKIKLNFERPTLLNGWDANKEEANLGVILLKEGKYYLGIINPYCKKIFEVDEKDSNEQNNYKKMEYKLLPGPNKMLPKVFFSNSRIEEFNPSKELQEKYNKGYHKKGKDFDINFCHELIDFYKQSLNKHEDWKKFNFKFKDTSEYNDISEFYREVEKQGYKIEYTEYSEKYINELVDRGELYLFQIYNKDFSEYSKGKENLHTLYWKAVFDPDNIMNPVYKLNGNAEVFYRKKSLEMKVTHPANQPIANKNISTIEAGRSTSTFKYDLIKDKRYTMDKFQFHVPITMNFKSERLLNINQIVNKYLKYNDDIHVIGIDRGERNLLYVCVIDKNEKIVYQKSLNEIVSEYNNNRYTTDYHGLLDRKEKEREIAREDWKNIENIKELKEGYMSQVIHILVELMKKYNAIIVIEDLNKGFKNSRIKVEKQVYQKFEKMFIDKLNYLVFKDEDKMDEGGVLNAYQLTNKFESFTKLGKQSGILYYIPAWCTSKIDPTTGFINRFYLKYENFDKSKEFVNRIDDIRYNEKENLFEFDIDYSKFTDRLNDTKNKWTLCSYGERILTQKNANGEWFDRRIQLSIEFKKLFEKYGINLNNIKDSILKLDKDNLEFYKGNGESLGFIQLFKLMVQMRNSLTGKEEDNLISPVKNQHGKFFNTSEKIEGLPIDADANGAYNIARKGFMLVEQMKNVEDEKLNKIKYNITEKEWLNYVQNRGM

SEQ ID NO: 226 - protein, 1249 aa, synthetic construct
MDNLLTFSSFTNLYSLSKTLRFKLVPIGKTYDNIVASGILDEDRHRAESYKKVKVIIDDYHKAYIESVLDGYRLPVKSQGKLDSLEEYFYYYHLGQKSDKERDTFKKIQAALRKCIAKQLKNSERFSRIFKKELIQQDLIDFVSGSPEADNEISLISEFKNFTVYFTGFHRNRANMYSEEEKSTAIAYRLINENLPKFVDNIDVFRKIANTEIAENFKSLYKTFEEYLQVEKLEDLFSLDYFSFVLTQKHIDVYNAIIGGKTTEQGEKVQGLNELINIYNQHHKTEKLPKLKFLFKQILSDKEALSWLPDEIESDADLLNSIKDFYDNVVKNLVDNATLKLLMESIANYDLSGIFIRNDQQLTEISQRVAGGWGVISNAVKKYLEATNVQKKKETFENYEERIDKLFKSYPSFSIAFLNQTLAESGLPNVRIEDYFRQSGAVNSDTVQKENLFARITNAYTMVESLLKCQLPSGYNLAQDSQSVALIKQLLDSIKELQLFIKPLLGAGDEADKNETFYGDFCEYWNELDILTPMYNKVRNYLTKKPYSEDKIKLNFKNPTLLDGWDLNKESDNLSVILRRDGSYFLAIMDRNFRKAFVNTPSLDLGEPYYEKMEYKLLPGPNKMLPKVFFSKSRVAEFNPSKELLDKYERGTHKKGDNFNLSDCHDLIDFFKASIQKHDDWKGFGFKFSETSSYSDLSGFYREVEMQGYKLSFAKVSADYVEKLVDEDKLYLFQIYNKDFSSYSKGTPNMHTLYWKMLFDERNLADVVYKLNGQAEVFFRKASLQQGKPTHPANVPLKNKNSLNPKSESVFGYDLIKDKRYTVDQFQLHVPITLNFKTAGQSNINQLVNDYIRTADDLHVIGIDRGERHLLYLVVIDSKGKICEQFSLNEIVNECKGISYKTNYHDLLNQKEQERLNARRSWQSIANIKELKEGYLSQVVHKIAELMVKYKAIVVLEDLNTGFKRGRQKVEKQVYQKFEKMLIDKLNFLAFKNENPELPGGILHAYQLTNKFDSFVNLGKQSGFLFYIPAWNTSKIDPMTGFVNLFDVKYESVEKAKAFFSRFDFIRYQSDKDWFEFEFDYNHFTQKAEGTKTHWTLCTYGTRIRTFRNVAKNSQWDNEEINLTDAFKSLFEKFNIDRHSNLKEELSEISEKEFFVELIHLFKLTLQMRNSVTGTDIDYLISPVMAKNGEFYDSRKADDSLPCNADANGAYNIARKGLMLQERIRMSDDQKKINFKITNKEWLDFAQNL

SEQ ID NO: 227 - protein, 1249 aa, synthetic construct
MDNLLTFSSFTNLYSLSKTLRFKLVPIGKTYDNIVASGILDEDRHRAESYKKVKVIIDDYHKAYIESVLDGYRLPVKSQGKLDSLEEYFYYYHLGQKSDKERDTFKKIQAALRKCIAKQLKNSERFSRIFKKELIQQDLIDFVSGSPEADNEISLISEFKNFTVYFTGFHENRANMYSEEEKSTAIAYRLINENLPKFVDNIDVFRKIANTEIAENFKSLYKTFEEYLQVEKLEDLFSLDYFSFVLTQKHIDVYNAIIGGKTTEQGEKVQGLNELINIYNQHHKTEKLPKLKFLFKQILSDKEALSWLPDEIESDADLLNSIKDFYDNVVKNLVDNATLKLLMESIANYDLSGIFIRNDQQLTEISQRVAGGWGVISNAVKKYLEATNVQKKKETFENYEERIDKLFKSYPSFSIAFLNQTLAESGLPNVRIEDYFRQSGAVNSDTVQKENLFARITNAYTMVESLLKCQLPSGYNLAQDSQSVALIKQLLDSIKELQLFIKPLLGAGDEADKNETFYGDFCEYWNELDILTPMYNKVRNYLTKKPYSEDKIKLNFKNPTLLRGWDLNKESDNLSVILRRDGSYFLAIMDRNFRKAFVNTPSLDLGEPYYEKMEYKLLPGPNKMLPKVFFSKSRVAEFNPSKELLDKYERGTHKKGDNFNLSDCHDLIDFFKASIQKHDDWKGFGFKFSETSSYSDLSGFYREVEMQGYKLSFAKVSADYVEKLVDEDKLYLFQIYNKDFSSYSKGTPNMHTLYWKMLFDERNLADVVYKLNGQAEVFFRKASLQQGKPTHPANVPLKNKNSLNPKSESVFGYDLIKDKRYTVDQFQLHVPITLNFKTAGQSNINQLVNDYIRTADDLHVIGIDRGERHLLYLVVIDSKGKICEQFSLNEIVNECKGISYKTNYHDLLNQKEQERLNARRSWQSIANIKELKEGYLSQVVHKIAELMVKYKAIVVLEDLNTGFKRGRQKVEKQVYQKFEKMLIDKLNFLAFKNENPELPGGILHAYQLTNKFDSFVNLGKQSGFLFYIPAWNTSKIDPMTGFVNLFDVKYESVEKAKAFFSRFDFIRYQSDKDWFEFEFDYNHFTQKAEGTKTHWTLCTYGTRIRTFRNVAKNSQWDNEEINLTDAFKSLFEKFNIDRHSNLKEELSEISEKEFFVELIHLFKLTLQMRNSVTGTDIDYLISPVMAKNGEFYDSRKADDSLPCNADANGAYNIARKGLMLQERIRMSDDQKKINFKITNKEWLDFAQNL

SEQ ID NO: 228 - protein, 1249 aa, synthetic construct
MDNLLTFSSFTNLYSLSKTLRFKLVPIGKTYDNIVASGILDEDRHRAESYKKVKVIIDDYHKAYIESVLDGYRLPVKSQGKLDSLEEYFYYYHLGQKSDKERDTFKKIQAALRKCIAKQLKNSERFSRIFKKELIQQDLIDFVSGSPEADNEISLISEFKNFTVYFTGFHENRANMYSEEEKSTAIAYRLINENLPKFVDNIDVFRKIANTEIAENFKSLYKTFEEYLQVEKLEDLFSLDYFSFVLTQKHIDVYNAIIGGKTTEQGEKVQGLNELINIYNQHHKTEKLPKLKFLFKQILSDKEALSWLPDEIESDADLLNSIKDFYDNVVKNLVDNATLKLLMESIANYDLSGIFIRNDQQLTEISQRVAGGWGVISNAVKKYLEATNVQKKKETFENYEERIDKLFKSYPSFSIAFLNQTLAESGLPNVRIEDYFRQSGAVNSDTVQKENLFARITNAYTMVESLLKCQLPSGYNLAQDSQSVALIKQLLDSIKELQLFIKPLLGAGDEADKNETFYGDFCEYWNELDILTPMYNKVRNYLTKKPYSEDKIKLNFKNPTLLDGWDLNRESDNLSVILRRDGSYFLAIMDRNFRKAFVNTPSLDLGEPYYEKMEYKLLPGPNKMLPKVFFSKSRVAEFNPSKELLDKYERGTHKKGDNFNLSDCHDLIDFFKASIQKHDDWKGFGFKFSETSSYSDLSGFYREVEMQGYKLSFAKVSADYVEKLVDEDKLYLFQIYNKDFSSYSKGTPNMHTLYWKMLFDERNLADVVYKLNGQAEVFFRKASLQQGKPTHPANVPLKNKNSLNPKSESVFGYDLIKDKRYTVDQFQLHVPITLNFKTAGQSNINQLVNDYIRTADDLHVIGIDRGERHLLYLVVIDSKGKICEQFSLNEIVNECKGISYKTNYHDLLNQKEQERLNARRSWQSIANIKELKEGYLSQVVHKIAELMVKYKAIVVLEDLNTGFKRGRQKVEKQVYQKFEKMLIDKLNFLAFKNENPELPGGILHAYQLTNKFDSFVNLGKQSGFLFYIPAWNTSKIDPMTGFVNLFDVKYESVEKAKAFFSRFDFIRYQSDKDWFEFEFDYNHFTQKAEGTKTHWTLCTYGTRIRTFRNVAKNSQWDNEEINLTDAFKSLFEKFNIDRHSNLKEELSEISEKEFFVELIHLFKLTLQMRNSVTGTDIDYLISPVMAKNGEFYDSRKADDSLPCNADANGAYNIARKGLMLQERIRMSDDQKKINFKITNKEWLDFAQNL

SEQ ID NO: 229 - protein, 1249 aa, synthetic construct
MDNLLTFSSFTNLYSLSKTLRFKLVPIGKTYDNIVASGILDEDRHRAESYKKVKVIIDDYHKAYIESVLDGYRLPVKSQGKLDSLEEYFYYYHLGQKSDKERDTFKKIQAALRKCIAKQLKNSERFSRIFKKELIQQDLIDFVSGSPEADNEISLISEFKNFTVYFTGFHRNRANMYSEEEKSTAIAYRLINENLPKFVDNIDVFRKIANTEIAENFKSLYKTFEEYLQVEKLEDLFSLDYFSFVLTQKHIDVYNAIIGGKTTEQGEKVQGLNELINIYNQHHKTEKLPKLKFLFKQILSDKEALSWLPDEIESDADLLNSIKDFYDNVVKNLVDNATLKLLMESIANYDLSGIFIRNDQQLTEISQRVAGGWGVISNAVKKYLEATNVQKKKETFENYEERIDKLFKSYPSFSIAFLNQTLAESGLPNVRIEDYFRQSGAVNSDTVQKENLFARITNAYTMVESLLKCQLPSGYNLAQDSQSVALIKQLLDSIKELQLFIKPLLGAGDEADKNETFYGDFCEYWNELDILTPMYNKVRNYLTKKPYSEDKIKLNFKNPTLLRGWDLNKESDNLSVILRRDGSYFLAIMDRNFRKAFVNTPSLDLGEPYYEKMEYKLLPGPNKMLPKVFFSKSRVAEFNPSKELLDKYERGTHKKGDNFNLSDCHDLIDFFKASIQKHDDWKGFGFKFSETSSYSDLSGFYREVEMQGYKLSFAKVSADYVEKLVDEDKLYLFQIYNKDFSSYSKGTPNMHTLYWKMLFDERNLADVVYKLNGQAEVFFRKASLQQGKPTHPANVPLKNKNSLNPKSESVFGYDLIKDKRYTVDQFQLHVPITLNFKTAGQSNINQLVNDYIRTADDLHVIGIDRGERHLLYLVVIDSKGKICEQFSLNEIVNECKGISYKTNYHDLLNQKEQERLNARRSWQSIANIKELKEGYLSQVVHKIAELMVKYKAIVVLEDLNTGFKRGRQKVEKQVYQKFEKMLIDKLNFLAFKNENPELPGGILHAYQLTNKFDSFVNLGKQSGFLFYIPAWNTSKIDPMTGFVNLFDVKYESVEKAKAFFSRFDFIRYQSDKDWFEFEFDYNHFTQKAEGTKTHWTLCTYGTRIRTFRNVAKNSQWDNEEINLTDAFKSLFEKFNIDRHSNLKEELSEISEKEFFVELIHLFKLTLQMRNSVTGTDIDYLISPVMAKNGEFYDSRKADDSLPCNADANGAYNIARKGLMLQERIRMSDDQKKINFKITNKEWLDFAQNL

SEQ ID NO: 230 - protein, 1249 aa, synthetic construct
MDNLLTFSSFTNLYSLSKTLRFKLVPIGKTYDNIVASGILDEDRHRAESYKKVKVIIDDYHKAYIESVLDGYRLPVKSQGKLDSLEEYFYYYHLGQKSDKERDTFKKIQAALRKCIAKQLKNSERFSRIFKKELIQQDLIDFVSGSPEADNEISLISEFKNFTVYFTGFHRNRANMYSEEEKSTAIAYRLINENLPKFVDNIDVFRKIANTEIAENFKSLYKTFEEYLQVEKLEDLFSLDYFSFVLTQKHIDVYNAIIGGKTTEQGEKVQGLNELINIYNQHHKTEKLPKLKFLFKQILSDKEALSWLPDEIESDADLLNSIKDFYDNVVKNLVDNATLKLLMESIANYDLSGIFIRNDQQLTEISQRVAGGWGVISNAVKKYLEATNVQKKKETFENYEERIDKLFKSYPSFSIAFLNQTLAESGLPNVRIEDYFRQSGAVNSDTVQKENLFARITNAYTMVESLLKCQLPSGYNLAQDSQSVALIKQLLDSIKELQLFIKPLLGAGDEADKNETFYGDFCEYWNELDILTPMYNKVRNYLTKKPYSEDKIKLNFKNPTLLDGWDLNRESDNLSVILRRDGSYFLAIMDRNFRKAFVNTPSLDLGEPYYEKMEYKLLPGPNKMLPKVFFSKSRVAEFNPSKELLDKYERGTHKKGDNFNLSDCHDLIDFFKASIQKHDDWKGFGFKFSETSSYSDLSGFYREVEMQGYKLSFAKVSADYVEKLVDEDKLYLFQIYNKDFSSYSKGTPNMHTLYWKMLFDERNLADVVYKLNGQAEVFFRKASLQQGKPTHPANVPLKNKNSLNPKSESVFGYDLIKDKRYTVDQFQLHVPITLNFKTAGQSNINQLVNDYIRTADDLHVIGIDRGERHLLYLVVIDSKGKICEQFSLNEIVNECKGISYKTNYHDLLNQKEQERLNARRSWQSIANIKELKEGYLSQVVHKIAELMVKYKAIVVLEDLNTGFKRGRQKVEKQVYQKFEKMLIDKLNFLAFKNENPELPGGILHAYQLTNKFDSFVNLGKQSGFLFYIPAWNTSKIDPMTGFVNLFDVKYESVEKAKAFFSRFDFIRYQSDKDWFEFEFDYNHFTQKAEGTKTHWTLCTYGTRIRTFRNVAKNSQWDNEEINLTDAFKSLFEKFNIDRHSNLKEELSEISEKEFFVELIHLFKLTLQMRNSVTGTDIDYLISPVMAKNGEFYDSRKADDSLPCNADANGAYNIARKGLMLQERIRMSDDQKKINFKITNKEWLDFAQNL

SEQ ID NO: 231 - protein, 1249 aa, synthetic construct
MDNLLTFSSFTNLYSLSKTLRFKLVPIGKTYDNIVASGILDEDRHRAESYKKVKVIIDDYHKAYIESVLDGYRLPVKSQGKLDSLEEYFYYYHLGQKSDKERDTFKKIQAALRKCIAKQLKNSERFSRIFKKELIQQDLIDFVSGSPEADNEISLISEFKNFTVYFTGFHENRANMYSEEEKSTAIAYRLINENLPKFVDNIDVFRKIANTEIAENFKSLYKTFEEYLQVEKLEDLFSLDYFSFVLTQKHIDVYNAIIGGKTTEQGEKVQGLNELINIYNQHHKTEKLPKLKFLFKQILSDKEALSWLPDEIESDADLLNSIKDFYDNVVKNLVDNATLKLLMESIANYDLSGIFIRNDQQLTEISQRVAGGWGVISNAVKKYLEATNVQKKKETFENYEERIDKLFKSYPSFSIAFLNQTLAESGLPNVRIEDYFRQSGAVNSDTVQKENLFARITNAYTMVESLLKCQLPSGYNLAQDSQSVALIKQLLDSIKELQLFIKPLLGAGDEADKNETFYGDFCEYWNELDILTPMYNKVRNYLTKKPYSEDKIKLNFKNPTLLRGWDLNRESDNLSVILRRDGSYFLAIMDRNFRKAFVNTPSLDLGEPYYEKMEYKLLPGPNKMLPKVFFSKSRVAEFNPSKELLDKYERGTHKKGDNFNLSDCHDLIDFFKASIQKHDDWKGFGFKFSETSSYSDLSGFYREVEMQGYKLSFAKVSADYVEKLVDEDKLYLFQIYNKDFSSYSKGTPNMHTLYWKMLFDERNLADVVYKLNGQAEVFFRKASLQQGKPTHPANVPLKNKNSLNPKSESVFGYDLIKDKRYTVDQFQLHVPITLNFKTAGQSNINQLVNDYIRTADDLHVIGIDRGERHLLYLVVIDSKGKICEQFSLNEIVNECKGISYKTNYHDLLNQKEQERLNARRSWQSIANIKELKEGYLSQVVHKIAELMVKYKAIVVLEDLNTGFKRGRQKVEKQVYQKFEKMLIDKLNFLAFKNENPELPGGILHAYQLTNKFDSFVNLGKQSGFLFYIPAWNTSKIDPMTGFVNLFDVKYESVEKAKAFFSRFDFIRYQSDKDWFEFEFDYNHFTQKAEGTKTHWTLCTYGTRIRTFRNVAKNSQWDNEEINLTDAFKSLFEKFNIDRHSNLKEELSEISEKEFFVELIHLFKLTLQMRNSVTGTDIDYLISPVMAKNGEFYDSRKADDSLPCNADANGAYNIARKGLMLQERIRMSDDQKKINFKITNKEWLDFAQNL

SEQ ID NO: 232 - protein, 1249 aa, synthetic construct
MDNLLTFSSFTNLYSLSKTLRFKLVPIGKTYDNIVASGILDEDRHRAESYKKVKVIIDDYHKAYIESVLDGYRLPVKSQGKLDSLEEYFYYYHLGQKSDKERDTFKKIQAALRKCIAKQLKNSERFSRIFKKELIQQDLIDFVSGSPEADNEISLISEFKNFTVYFTGFHRNRANMYSEEEKSTAIAYRLINENLPKFVDNIDVFRKIANTEIAENFKSLYKTFEEYLQVEKLEDLFSLDYFSFVLTQKHIDVYNAIIGGKTTEQGEKVQGLNELINIYNQHHKTEKLPKLKFLFKQILSDKEALSWLPDEIESDADLLNSIKDFYDNVVKNLVDNATLKLLMESIANYDLSGIFIRNDQQLTEISQRVAGGWGVISNAVKKYLEATNVQKKKETFENYEERIDKLFKSYPSFSIAFLNQTLAESGLPNVRIEDYFRQSGAVNSDTVQKENLFARITNAYTMVESLLKCQLPSGYNLAQDSQSVALIKQLLDSIKELQLFIKPLLGAGDEADKNETFYGDFCEYWNELDILTPMYNKVRNYLTKKPYSEDKIKLNFKNPTLLRGWDLNRESDNLSVILRRDGSYFLAIMDRNFRKAFVNTPSLDLGEPYYEKMEYKLLPGPNKMLPKVFFSKSRVAEFNPSKELLDKYERGTHKKGDNFNLSDCHDLIDFFKASIQKHDDWKGFGFKFSETSSYSDLSGFYREVEMQGYKLSFAKVSADYVEKLVDEDKLYLFQIYNKDFSSYSKGTPNMHTLYWKMLFDERNLADVVYKLNGQAEVFFRKASLQQGKPTHPANVPLKNKNSLNPKSESVFGYDLIKDKRYTVDQFQLHVPITLNFKTAGQSNINQLVNDYIRTADDLHVIGIDRGERHLLYLVVIDSKGKICEQFSLNEIVNECKGISYKTNYHDLLNQKEQERLNARRSWQSIANIKELKEGYLSQVVHKIAELMVKYKAIVVLEDLNTGFKRGRQKVEKQVYQKFEKMLIDKLNFLAFKNENPELPGGILHAYQLTNKFDSFVNLGKQSGFLFYIPAWNTSKIDPMTGFVNLFDVKYESVEKAKAFFSRFDFIRYQSDKDWFEFEFDYNHFTQKAEGTKTHWTLCTYGTRIRTFRNVAKNSQWDNEEINLTDAFKSLFEKFNIDRHSNLKEELSEISEKEFFVELIHLFKLTLQMRNSVTGTDIDYLISPVMAKNGEFYDSRKADDSLPCNADANGAYNIARKGLMLQERIRMSDDQKKINFKITNKEWLDFAQNL

SEQ ID NO: 233 - protein, 1249 aa, synthetic construct
MDNLLTFSSFTNLYSLSKTLRFKLVPIGKTYDNIVASGILDEDRHRAESYKKVKVIIDDYHKAYIESVLDGYRLPVKSQGKLDSLEEYFYYYHLGQKSDKERDTFKKIQAALRKCIAKQLKNSERFSRIFKKELIQQDLIDFVSGSPEADNEISLISEFKNFTVYFTGFHRNRANMYSEEEKSTAIAYRLINENLPKFVDNIDVFRKIANTEIAENFKSLYKTFEEYLQVEKLEDLFSLDYFSFVLTQKHIDVYNAIIGGKTTEQGEKVQGLNELIAIYNQHHKTEKLPKLKFLFKQILSDKEALSWLPDEIESDADLLNSIKDFYDNVVKNLVDNATLKLLMESIANYDLSGIFIRNDQQLTEISQRVAGGWGVISNAVKKYLEATNVQKKKETFENYEERIDKLFKSYPSFSIAFLNQTLAESGLPNVRIEDYFRQSGAVNSDTVQKENLFARITNAYTMVESLLKCQLPSGYNLAQDSQSVALIKQLLDSIKELQLFIKPLLGAGDEADKNETFYGDFCEYWNELDILTPMYNKVRNYLTKKPYSEDKIKLNFKNPTLLDGWDLNRESDNLSVILRRDGSYFLAIMDRNFRKAFVNTPSLDLGEPYYEKMEYKLLPGPNKMLPKVFFSKSRVAEFNPSKELLDKYERGTHKKGDNFNLSDCHDLIDFFKASIQKHDDWKGFGFKFSETSSYSDLSGFYREVEMQGYKLSFAKVSADYVEKLVDEDKLYLFQIYNKDFSSYSKGTPNMHTLYWKMLFDERNLADVVYKLNGQAEVFFRKASLQQGKPTHPANVPLKNKNSLNPKSESVFGYDLIKDKRYTVDQFQLHVPITLNFKTAGQSNINQLVNDYIRTADDLHVIGIDRGERHLLYLVVIDSKGKICEQFSLNEIVNECKGISYKTNYHDLLNQKEQERLNARRSWQSIANIKELKEGYLSQVVHKIAELMVKYKAIVVLEDLNTGFKRGRQKVEKQVYQKFEKMLIDKLNFLAFKNENPELPGGILHAYQLTNKFDSFVNLGKQSGFLFYIPAWNTSKIDPMTGFVNLFDVKYESVEKAKAFFSRFDFIRYQSDKDWFEFEFDYNHFTQKAEGTKTHWTLCTYGTRIRTFRNVAKNSQWDNEEINLTDAFKSLFEKFNIDRHSNLKEELSEISEKEFFVELIHLFKLTLQMRNSVTGTDIDYLISPVMAKNGEFYDSRKADDSLPCNADANGAYNIARKGLMLQERIRMSDDQKKINFKITNKEWLDFAQNL

SEQ ID NO: 234 - protein, 1249 aa, synthetic construct
MDNLLTFSSFTNLYSLSKTLRFKLVPIGKTYDNIVASGILDEDRHRAESYKKVKVIIDDYHKAYIESVLDGYRLPVKSQGKLDSLEEYFYYYHLGQKSDKERDTFKKIQAALRKCIAKQLKNSERFSRIFKKELIQQDLIDFVSGSPEADNEISLISEFKNFTVYFTGFHENRANMYSEEEKSTAIAYRLINENLPKFVDNIDVFRKIANTEIAENFKSLYKTFEEYLQVEKLEDLFSLDYFSFVLTQKHIDVYNAIIGGKTTEQGEKVQGLNELINIYNQHHKTEKLPKLKFLFKQILSDKEALSWLPDEIESDADLLNSIKDFYDNVVKNLVDNATLKLLMESIANYDLSGIFIRNDQQLTEISQRVAGGWGVISNAVKKYLEATNVQKKKETFENYEERIDKLFKSYPSFSIAFLNQTLAESGLPNVRIEDYFRQSGAVNSDTVQKENLFARITNAYTMVESLLKCQLPSGYNLAQDSQSVALIKQLLDSIKELQLFIKPLLGAGDEADKNETFYGDFCEYWNELDILTPMYNKVRNYLTKKPYSEDKIKLNFKRPTLLDGWDLNKESDNLSVILRRDGSYFLAIMDRNFRKAFVNTPSLDLGEPYYEKMEYKLLPGPNKMLPKVFFSKSRVAEFNPSKELLDKYERGTHKKGDNFNLSDCHDLIDFFKASIQKHDDWKGFGFKFSETSSYSDLSGFYREVEMQGYKLSFAKVSADYVEKLVDEDKLYLFQIYNKDFSSYSKGTPNMHTLYWKMLFDERNLADVVYKLNGQAEVFFRKASLQQGKPTHPANVPLKNKNSLNPKSESVFGYDLIKDKRYTVDQFLLHVPITLNFKTAGQSNINQLVNDYIRTADDLHVIGIDRGERHLLYLVVIDSKGKICEQFSLNEIVNECKGISYKTNYHDLLNQKEQERLNARRSWQSIANIKELKEGYLSQVVHKIAELMVKYKAIVVLEDLNTGFKRGRQKVEKQVYQKFEKMLIDKLNFLAFKNENPELPGGILHAYQLTNKFDSFVNLGKQSGFLFYIPAWNTSKIDPMTGFVNLFDVKYESVEKAKAFFSRFDFIRYQSDKDWFEFEFDYNHFTQKAEGTKTHWTLCTYGTRIRTFRNVAKNSQWDNEEINLTDAFKSLFEKFNIDRHSNLKEELSEISEKEFFVELIHLFKLTLQMRNSVTGTDIDYLISPVMAKNGEFYDSRKADDSLPCNADANGAYNIARKGLMLQERIRMSDDQKKINFKITNKEWLDFAQNL

SEQ ID NO: 235 - protein, 1221 aa, synthetic construct
MEKFYDKFNNLFSVSKTLRFELEPIGKTKEFFEKYILKEDERKSDLFKKVKKYCDEYHKLFISECLKNFDDKDFENLLKEYFALVNLSNPSEKETKRITDILKVLRAKISDRFTKNPKYKGLFGKELINDYLKDYYKNNKNILDEITPFKDFTSYFTGFNRNRKNMYSKEEKHTAIAYRLIDENLSTYIKNIKAFNIILENIPDIKIQIKENLNLDCDEFFSNIENYTNVLTQEQIETYNLAISGKSQENSKIKGINEIVALYKQKNKIKIPKLKELYKQLLSDTTSASFKFDIIEYDYQIVEMINSYYETFKKNFIDDNGCQSALSNIQNYDIKNIYINNDVSLTSLSQNIYNDWNYINSLLGNEYDQNYKGKAKFGTEKYSEKKEETLKKIKEISLLKIEELIKNYDKENNNSEKIFEYFKNAIEENLKKIDIEYKNTKTILQKEYDKTSTDLLKDEQSIEKIKNFLDSIKDLQILIKYLIPKNNTLETDLEFYNALNYDILSEIIPVYNKTRNYLTKKTYSLEKFKLNFDCPTFLNGWDLNKEEANLGTLFRKDGNYYIGIMNTNSRKSFIEYNQAEFKNEKKYQKIEYKLLPNPNQMLPKVFFSESRIKEFNPSEELLSKYERGYHKKGADFDLNFCHQLIDFFKSSINKHEDWSKFNFKFSETNKYNDISEFYKEVELQGYKISFTDISEEYINKLVEEGKLYLFRIYNKDFSSYSKGTPNLHTLYWKALFDEDNLKNIIYKLNGGAEIFYRKKSIEAKITHPKNQPIDNKNPNNPKKQSIFEYDLIKDKRFTLDKFQFHVPITLNFKAKDTHKLNDLVNNKIKNTEDINIIGIDRGERHLLYACVINKNGEILEQYSLNTIGQTNYHYLLSKKEDDRLKQRQAWGTIQNIKELKEGYMSQVIHKLTKLMVQYNAVIVLEDLNSGFKNSRKKVEKSVYDKFETALINKLSYLVDKSIQNKFEQGGIMNAYQLANADIKSAKQNGVIFYIPAWCTSKIDPTTGFVNLFDLKKIDKEFVKKFNSIKFNNTENYFEFDFDYSNFSDKSSGDRTNWTLCSFGNRIRTFKNPKKNNNYDSQTVELTQEFKKLFDCYNLDCSNLRDEILNKSDSKFFNATPEKNNFYGFALLFKLLLQLRNSITGSEEDYILSPIKNKKGLFYDSRTANGVLPENADANGAYNIARKGLLILNRIKNSEENTKTDYTIKNADWLTYVQQQDR

SEQ ID NO: 236 - protein, 1205 aa, synthetic construct
MKLEDFTNLYSLSKTLRFELRPIGKTRENIENGGLLRQDEDRAEKYVHIKKLIDEYHKAYIDKQLSGLVLQYADIGKANSLEEYYHSTRKSKDSDKDKIVKIQDNLRKQIVKRLKDSDEFKRIDKKELIQSDLAEFIKPAEDRALIAEFKNFTTYFTGFNENRQNMYSDKAISTAIAYRLIHENLPKFIDNIETFDRIAGITELYDQTSSDAEIFRLAHFSETLSQKQIDAYNSVMGRYNMLINEYNQTHKQSRLPKFKMLYKQILSDREHPSWLPEQFESDTAVLTAIRECYDDLRIPMANLKTLLEGLGNYDPSGIFLRNDQHLSQISKRLTGDRSSIERSVTEDLLTSRKLNKRKSRTTDEEESRKLFKQKGSLSIGYIADTAKIDVERYFAKLGAINTVTEQSENLFAKAENARTTADELLANDYPAGKRLVQSNDDIALVKNLLDALMELQWFVKPLLGTGDEAGKDERFYGEFTQIWEQLDRITPLYNMVRNYVTRKPYSTDKFKLNFESAALLGGWDKNREPDCLSVILRKDEQYYLGIINKNHKKIFARDILPCEGECYEKMVYKQISISAGIGGFFRKCYRTAEQYGWCCSDSCLNDERKIIIRDDEARENLTEIINCQKDFLNKYEKDGFKYRDFGFRFAASSDYEKLSDFYRDIDEQRYKLSFTPVSATYIDSLVEEGKLYLFQIYNKDFSAYSKGTPNMHTLYWRMLFDERNLSDVVYQLNGGAELFFRRKSLQNGRPTHPANIPIKNKNSRNDKKESLFDYDLIKDRRYTVDKFQFHVPITLNFKSDGAGRINERVREYLRSADDVHVIGIDRGERNLLYLVVTDMDGNICEQFSLNEICNTDYHSLLDEREHKRMQERQSWQAIEGIKELKEGYLSQVVHRIATLMVKYRAIVVLEDLNFGFMRSRQKVEKSVYQKFEHMLIDKLNYLVDKKANPTTPGGLLKAYQLTDKFESFQKLGKQSGFLFYVPAWNTSKIDPATGFVNMLDLGYESIDKAKALLCKFDSIRYNACKDWFEFALDYDKFGSKATGTRTKWTVCTYGQRIDTYRNKDSQWVSRDVDLTNELKSLFSEHGIDIYSNLKDAIVAQNDKEFFANMLRILKLTMQMRNSKTGTDIDYIVSPVADANGRFFDSRQADATMPKDADANGAYNIARKGIMLVQQIKQSDDLRTMKFDISNKSWLRFVQHTNQADE

SEQ ID NO: 237 - DNA, 19 bp, synthetic construct
  source 1..19: mol_type = other DNA
aatttctactatcgtagat

SEQ ID NO: 238 - DNA, 36 bp, synthetic construct
  source 1..36: mol_type = other DNA
gactaaaacccccaacaaatttctacttttgtagat

SEQ ID NO: 239 - DNA, 19 bp, synthetic construct
  source 1..19: mol_type = other DNA
aatttctacttttgtagat

SEQ ID NO: 240 - DNA, 36 bp, synthetic construct
  source 1..36: mol_type = other DNA
atttaaacataatatctaatttctacttttgtagat

SEQ ID NO: 241 - DNA, 19 bp, synthetic construct
  source 1..19: mol_type = other DNA
aatttctacttttgtagat

SEQ ID NO: 242 - DNA, 36 bp, synthetic construct
  source 1..36: mol_type = other DNA
gtttaatacctatttaaaatttctactattgtagat

SEQ ID NO: 243 - DNA, 19 bp, synthetic construct
  source 1..19: mol_type = other DNA
aatttctactattgtagat

SEQ ID NO: 244 - DNA, 36 bp, synthetic construct
  source 1..36: mol_type = other DNA
gtttaataaccctatataatttctactattgtagat

SEQ ID NO: 245 - DNA, 19 bp, synthetic construct
  source 1..19: mol_type = other DNA
aatttctactattgtagat

SEQ ID NO: 246 - DNA, 36 bp, synthetic construct
  source 1..36: mol_type = other DNA
ggctatacgcctacgaaaatttctactcttgtagat

SEQ ID NO: 247 - DNA, 19 bp, synthetic construct
  source 1..19: mol_type = other DNA
aatttctactcttgtagat

SEQ ID NO: 248 - DNA, 36 bp, synthetic construct
  source 1..36: mol_type = other DNA
ggctaacagccttttaaatttctactgtgtgtagat

SEQ ID NO: 249 - DNA, 20 bp, synthetic construct
  source 1..20: mol_type = other DNA
aatttctactgtgtgtagat

SEQ ID NO: 250 - DNA, 35 bp, synthetic construct
  source 1..35: mol_type = other DNA
cttaatagctgatataaatttctactgttgtagat

SEQ ID NO: 251 - DNA, 19 bp, synthetic construct
  source 1..19: mol_type = other DNA
aatttctactgttgtagat

SEQ ID NO: 252 - DNA, 36 bp, synthetic construct
  source 1..36: mol_type = other DNA
gtttattccttatagtagatttctacttttgtagat

SEQ ID NO: 253 - DNA, 19 bp, synthetic construct
  source 1..19: mol_type = other DNA
gatttctacttttgtagat

SEQ ID NO: 254 - DNA, 36 bp, synthetic construct
  source 1..36: mol_type = other DNA
gtttaatgccttatataaatttctactattgtagat

SEQ ID NO: 255 - DNA, 19 bp, synthetic construct
  source 1..19: mol_type = other DNA
aatttctactattgtagat

SEQ ID NO: 256 - DNA, 36 bp, synthetic construct
  source 1..36: mol_type = other DNA
gtttaataccttatataaatttctactattgtagat

SEQ ID NO: 257 - DNA, 19 bp, synthetic construct
  source 1..19: mol_type = other DNA
aatttctactattgtagat

SEQ ID NO: 258 - DNA, 36 bp, synthetic construct; identical to SEQ ID NO: 256
  source 1..36: mol_type = other DNA

SEQ ID NO: 259 - DNA, 19 bp, synthetic construct
  source 1..19: mol_type = other DNA
aatttctactattgtagat

SEQ ID NO: 260 - DNA, 36 bp, synthetic construct
  source 1..36: mol_type = other DNA
gtctaggacaatatgaaaatttctactattgtagat

SEQ ID NO: 261 - DNA, 19 bp, synthetic construct
  source 1..19: mol_type = other DNA
aatttctactattgtagat

SEQ ID NO: 262 - DNA, 36 bp, synthetic construct
  source 1..36: mol_type = other DNA
gtctaatttttacggtcaatttctactaatgtagat

SEQ ID NO: 263 - DNA, 19 bp, synthetic construct
  source 1..19: mol_type = other DNA
aatttctactaatgtagat

SEQ ID NO: 264 - DNA, 36 bp, synthetic construct
  source 1..36: mol_type = other DNA
gtctaaaggtaccaccaaatttctactgttgtagat

SEQ ID NO: 265 - DNA, 19 bp, synthetic construct
  source 1..19: mol_type = other DNA
aatttctactgttgtagat

SEQ ID NO: 266 - DNA, 36 bp, synthetic construct
  source 1..36: mol_type = other DNA
gattaataatccctaataatttctactgttgtagat

SEQ ID NO: 267 - DNA, 19 bp, synthetic construct
  source 1..19: mol_type = other DNA
aatttctactgttgtagat

SEQ ID NO: 268 - DNA, 36 bp, synthetic construct
  source 1..36: mol_type = other DNA
gactaataagccaaatgaatttctactattgtagat

SEQ ID NO: 269 - DNA, 19 bp, synthetic construct
  source 1..19: mol_type = other DNA
aatttctactattgtagat

SEQ ID NO: 270 - DNA, 36 bp, synthetic construct
  source 1..36: mol_type = other DNA
ggctataggccttttataatttctactattgtagat

SEQ ID NO: 271 - DNA, 19 bp, synthetic construct
  source 1..19: mol_type = other DNA
aatttctactattgtagat

SEQ ID NO: 272 - DNA, 36 bp, synthetic construct; identical to SEQ ID NO: 256
  source 1..36: mol_type = other DNA

SEQ ID NO: 273 - DNA, 19 bp, synthetic construct
  source 1..19: mol_type = other DNA
aatttctactattgtagat

SEQ ID NO: 274 - DNA, 36 bp, synthetic construct
  source 1..36: mol_type = other DNA
gcctaataaggtaatgtaatttctactattgtagat

SEQ ID NO: 275 - DNA, 19 bp, synthetic construct
  source 1..19: mol_type = other DNA
aatttctactattgtagat

SEQ ID NO: 276 - DNA, 36 bp, synthetic construct; identical to SEQ ID NO: 248
  source 1..36: mol_type = other DNA

SEQ ID NO: 277 - DNA, 20 bp, synthetic construct
  source 1..20: mol_type = other DNA
aatttctactgtgtgtagat

SEQ ID NO: 278 - DNA, 36 bp, synthetic construct
  source 1..36: mol_type = other DNA
ataggaacaggtatcctaaacggatgctgatgtgac

SEQ ID NO: 279 - DNA, 36 bp, synthetic construct
  source 1..36: mol_type = other DNA
gtgctgttgatcttcacaaggggatcaataaggcac

SEQ ID NO: 280 - DNA, 36 bp, synthetic construct; identical to SEQ ID NO: 266
  source 1..36: mol_type = other DNA

SEQ ID NO: 281 - DNA, 19 bp, synthetic construct
  source 1..19: mol_type = other DNA
aatttctactgttgtagat

SEQ ID NO: 282 - DNA, 36 bp, synthetic construct; identical to SEQ ID NO: 266
  source 1..36: mol_type = other DNA

SEQ ID NO: 283 - DNA, 19 bp, synthetic construct
  source 1..19: mol_type = other DNA
aatttctactgttgtagat

SEQ ID NO: 284 - DNA, 36 bp, synthetic construct; identical to SEQ ID NO: 266
  source 1..36: mol_type = other DNA

SEQ ID NO: 285 - DNA, 19 bp, synthetic construct
  source 1..19: mol_type = other DNA
aatttctactgttgtagat

SEQ ID NO: 286 - DNA, 36 bp, synthetic construct; identical to SEQ ID NO: 266
  source 1..36: mol_type = other DNA

SEQ ID NO: 287 - DNA, 36 bp, synthetic construct
  source 1..36: mol_type = other DNA
gtttataacttataaaaaatttctactattgtagat

SEQ ID NO: 288 - DNA, 19 bp, synthetic construct
  source 1..19: mol_type = other DNA
aatttctactattgtagat

SEQ ID NO: 289 - DNA, 24 bp, synthetic construct
  source 1..24: mol_type = other DNA
agatctgatggtccatgtctgtta

SEQ ID NO: 290 - DNA, 24 bp, synthetic construct
  source 1..24: mol_type = other DNA
agaataacagacatggaccatcag

SEQ ID NO: 291 - DNA, 24 bp, synthetic construct
  source 1..24: mol_type = other DNA
agatggcggggtccagttccggga

SEQ ID NO: 292 - DNA, 24 bp, synthetic construct
  source 1..24: mol_type = other DNA
agaatcccggaactggaccccgcc

SEQ ID NO: 293 - DNA, 24 bp, synthetic construct
  source 1..24: mol_type = other DNA
agatgtcggcatggccccattcgc

SEQ ID NO: 294 - DNA, 24 bp, synthetic construct
  source 1..24: mol_type = other DNA
agaagcgaatggggccatgccgac

SEQ ID NO: 295 - DNA, 27 bp, synthetic construct
  source 1..27: mol_type = other DNA
agatcaaaagtaagattcactttcata

SEQ ID NO: 296 - DNA, 27 bp, synthetic construct
  source 1..27: mol_type = other DNA
agaatatgaaagtgaatcttacttttg

SEQ ID NO: 297 - DNA, 27 bp, synthetic construct
  source 1..27: mol_type = other DNA
agatgtgctcaatgaaaggagataagg

SEQ ID NO: 298 - DNA, 27 bp, synthetic construct
  source 1..27: mol_type = other DNA
agaaccttatctcctttcattgagcac

SEQ ID NO: 299 - DNA, 23 bp, synthetic construct
  source 1..23: mol_type = other DNA
ccagaatgcacaaagtactgcac

SEQ ID NO: 300 - DNA, 21 bp, synthetic construct
  source 1..21: mol_type = other DNA
gccaaagcccgagagagtgcc

SEQ ID NO: 301 - DNA, 20 bp, synthetic construct
  source 1..20: mol_type = other DNA
ggagacgttcatgactggca

SEQ ID NO: 302 - DNA, 20 bp, synthetic construct
  source 1..20: mol_type = other DNA
cgcctgggtcttcatcagag

SEQ ID NO: 303 - DNA, 20 bp, synthetic construct
  source 1..20: mol_type = other DNA
ggagacgttcatgactggca

SEQ ID NO: 304 - DNA, 20 bp, synthetic construct
  source 1..20: mol_type = other DNA
cgcctgggtcttcatcagag

SEQ ID NO: 305 - DNA, 18 bp, synthetic construct
  source 1..18: mol_type = other DNA
cctccaaaagtgcaaggc

SEQ ID NO: 306 - DNA, 20 bp, synthetic construct
  source 1..20: mol_type = other DNA
ttcttccacacaaccaacca

SEQ ID NO: 307 - DNA, 20 bp, synthetic construct
  source 1..20: mol_type = other DNA
gcatactcgcatggctacct

SEQ ID NO: 308 - DNA, 20 bp, synthetic construct
  source 1..20: mol_type = other DNA
ctccctgcagccccttttta

SEQ ID NO: 309 - DNA, 20 bp, synthetic construct
  source 1..20: mol_type = other DNA
ctgatggtccatgtctgtta

SEQ ID NO: 310 - DNA, 20 bp, synthetic construct
  source 1..20: mol_type = other DNA
ggcggggtccagttccggga

SEQ ID NO: 311 - DNA, 20 bp, synthetic construct
  source 1..20: mol_type = other DNA
gtcggcatggccccattcgc

SEQ ID NO: 312 - DNA, 23 bp, synthetic construct
  source 1..23: mol_type = other DNA
caaaagtaagattcactttcata

SEQ ID NO: 313 - DNA, 23 bp, synthetic construct
  source 1..23: mol_type = other DNA
gtgctcaatgaaaggagataagg

SEQ ID NO: 314 - DNA, 20 bp, synthetic construct
  source 1..20: mol_type = other DNA
gtctccaaggtgaaagcgga

SEQ ID NO: 315 - DNA, 20 bp, synthetic construct
  source 1..20: mol_type = other DNA
gcgacaaaaggcagcaaaga

SEQ ID NO: 316 - DNA, 35 bp, synthetic construct
  source 1..35: mol_type = other DNA
ggctaacagccttttaaatttctactgttgtagat

SEQ ID NO: 317 - DNA, 36 bp, synthetic construct
  source 1..36: mol_type = other DNA
ggctaacagccttttaaattcccactgtgtgtgggt

SEQ ID NO: 318 - DNA, 34 bp, synthetic construct
  source 1..34: mol_type = other DNA
ggctaacagccttttaaatttctatgtgttagat

SEQ ID NO: 319 - DNA, 34 bp, synthetic construct
  source 1..34: mol_type = other DNA
ggctaacagccttttaaatttctactgtgtagat

SEQ ID NO: 320 - DNA, 32 bp, synthetic construct
  source 1..32: mol_type = other DNA
ggctaacagccttttaaatttctatgttagat

SEQ ID NO: 321 - DNA, 34 bp, synthetic construct
  source 1..34: mol_type = other DNA
ggctaacagccttttaaatttctctgtgtgagat

SEQ ID NO: 322 - DNA, 32 bp, synthetic construct
  source 1..32: mol_type = other DNA
ggctaacagccttttaaatttctctgtgagat

SEQ ID NO: 323 - DNA, 27 bp, synthetic construct
  source 1..27: mol_type = other DNA
agatctgatggtccatgtctgttactc

SEQ ID NO: 324 - DNA, 27 bp, synthetic construct
  source 1..27: mol_type = other DNA
agaagagtaacagacatggaccatcag

SEQ ID NO: 325 - DNA, 24 bp, synthetic construct
  source 1..24: mol_type = other DNA
agatctgatggtccatgtctgtta

SEQ ID NO: 326 - DNA, 24 bp, synthetic construct
  source 1..24: mol_type = other DNA
agaataacagacatggaccatcag

SEQ ID NO: 327 - DNA, 22 bp, synthetic construct
  source 1..22: mol_type = other DNA
agatctgatggtccatgtctgt

SEQ ID NO: 328 - DNA, 22 bp, synthetic construct
  source 1..22: mol_type = other DNA
agaaacagacatggaccatcag

SEQ ID NO: 329 - DNA, 21 bp, synthetic construct
  source 1..21: mol_type = other DNA
agatctgatggtccatgtctg

SEQ ID NO: 330 - DNA, 21 bp, synthetic construct
  source 1..21: mol_type = other DNA
agaacagacatggaccatcag

SEQ ID NO: 331 - DNA, 20 bp, synthetic construct
  source 1..20: mol_type = other DNA
agatctgatggtccatgtct

SEQ ID NO: 332 - DNA, 20 bp, synthetic construct
  source 1..20: mol_type = other DNA
agaaagacatggaccatcag

SEQ ID NO: 333 - DNA, 19 bp, synthetic construct
  source 1..19: mol_type = other DNA
agatctgatggtccatgtc

SEQ ID NO: 334 - DNA, 19 bp, synthetic construct
  source 1..19: mol_type = other DNA
agaagacatggaccatcag

SEQ ID NO: 335 - DNA, 50 bp, synthetic construct
  source 1..50: mol_type = other DNA
gaaacaccgaatttctactgtgtgtagatctgatggtccatgtctgttac

SEQ ID NO: 336 - DNA, 50 bp, synthetic construct
  source 1..50: mol_type = other DNA
ccccgccatctacacacagtagaaattgagtaacagacatggaccatcag

SEQ ID NO: 337 - DNA, 39 bp, synthetic construct
  source 1..39: mol_type = other DNA
tatatcttgtggaaaggacgaaacaccgaatttctactg

SEQ ID NO: 338 - DNA, 40 bp, synthetic construct
  source 1..40: mol_type = other DNA
aatttaatcccggaactggaccccgccatctacacacagt

SEQ ID NO: 339 - DNA, 50 bp, synthetic construct
  source 1..50: mol_type = other DNA
tctactgtgtgtagatcaaaagtaagattcactttcataaatttctactg

SEQ ID NO: 340 - DNA, 50 bp, synthetic construct
  source 1..50: mol_type = other DNA
ttatctcctttcattgagcacatctacacacagtagaaatttatgaaagt

SEQ ID NO: 341 - DNA, 40 bp, synthetic construct
  source 1..40: mol_type = other DNA
tccagttccgggattaaatttctactgtgtgtagatcaaa

SEQ ID NO: 342 - DNA, 40 bp, synthetic construct
  source 1..40: mol_type = other DNA
ctacacacagtagaaattccttatctcctttcattgagca

SEQ ID NO: 343 - DNA, 50 bp, synthetic construct
  source 1..50: mol_type = other DNA
atttttttgttttagagctagaaatagcaagttaaaataaggctagtccg

SEQ ID NO: 344 - DNA, 50 bp, synthetic construct
  source 1..50: mol_type = other DNA
tgtctgcagaattggcgcacgcgctaaaaacggactagccttattttaac

SEQ ID NO: 345 - DNA, 40 bp, synthetic construct
  source 1..40: mol_type = other DNA
ggaatttctactgtgtgtagatttttttgttttagagcta

SEQ ID NO: 346 - DNA, 40 bp, synthetic construct
  source 1..40: mol_type = other DNA
acggtacctctagagccatttgtctgcagaattggcgcac

SEQ ID NO: 347 - DNA, 28 bp, synthetic construct
  source 1..28: mol_type = other DNA
gtcctttccacaagatatataaagccaa

SEQ ID NO: 348 - DNA, 20 bp, synthetic construct
  source 1..20: mol_type = other DNA
aatggctctagaggtaccgt

SEQ ID NO: 349 - DNA, 24 bp, synthetic construct
  source 1..24: mol_type = other DNA
agatgtgatggtccatgtctgtta

SEQ ID NO: 350 - DNA, 24 bp, synthetic construct
  source 1..24: mol_type = other DNA
agaataacagacatggaccatcac

SEQ ID NO: 351 - DNA, 24 bp, synthetic construct
  source 1..24: mol_type = other DNA
agatcagatggtccatgtctgtta

SEQ ID NO: 352 - DNA, 24 bp, synthetic construct
  source 1..24: mol_type = other DNA
agaataacagacatggaccatctg

SEQ ID NO: 353 - DNA, 24 bp, synthetic construct
  source 1..24: mol_type = other DNA
agatctcatggtccatgtctgtta

SEQ ID NO: 354 - DNA, 24 bp, synthetic construct
  source 1..24: mol_type = other DNA
agaataacagacatggaccatgag

SEQ ID NO: 355 - DNA, 24 bp, synthetic construct
  source 1..24: mol_type = other DNA
agatctgttggtccatgtctgtta

SEQ ID NO: 356 - DNA, 24 bp, synthetic construct
  source 1..24: mol_type = other DNA
agaataacagacatggaccaacag

SEQ ID NO: 357 - DNA, 24 bp, synthetic construct
  source 1..24: mol_type = other DNA
agatctgaaggtccatgtctgtta

SEQ ID NO: 358 - DNA, 24 bp, synthetic construct
  source 1..24: mol_type = other DNA
agaataacagacatggaccttcag

SEQ ID NO: 359 - DNA, 24 bp, synthetic construct
  source 1..24: mol_type = other DNA
agatctgatcgtccatgtctgtta

SEQ ID NO: 360 - DNA, 24 bp, synthetic construct
  source 1..24: mol_type = other DNA
agaataacagacatggacgatcag

SEQ ID NO: 361 - DNA, 24 bp, synthetic construct
  source 1..24: mol_type = other DNA
agatctgatgctccatgtctgtta

SEQ ID NO: 362 - DNA, 24 bp, synthetic construct
  source 1..24: mol_type = other DNA
agaataacagacatggagcatcag

SEQ ID NO: 363 - DNA, 24 bp, synthetic construct
  source 1..24: mol_type = other DNA
agatctgatggaccatgtctgtta

SEQ ID NO: 364 - DNA, 24 bp, synthetic construct
  source 1..24: mol_type = other DNA
agaataacagacatggtccatcag

SEQ ID NO: 365 - DNA, 24 bp, synthetic construct
  source 1..24: mol_type = other DNA
agatctgatggtgcatgtctgtta

SEQ ID NO: 366 - DNA, 24 bp, synthetic construct
  source 1..24: mol_type = other DNA
agaataacagacatgcaccatcag

SEQ ID NO: 367 - DNA, 24 bp, synthetic construct
  source 1..24: mol_type = other DNA
agatctgatggtcgatgtctgtta

SEQ ID NO: 368 - DNA, 24 bp, synthetic construct
  source 1..24: mol_type = other DNA
agaataacagacatcgaccatcag

SEQ ID NO: 369 - DNA, 24 bp, synthetic construct
  source 1..24: mol_type = other DNA
agatctgatggtccttgtctgtta

SEQ ID NO: 370 - DNA, 24 bp, synthetic construct
  source 1..24: mol_type = other DNA
agaataacagacaaggaccatcag

SEQ ID NO: 371 - DNA, 24 bp, synthetic construct
  source 1..24: mol_type = other DNA
agatctgatggtccaagtctgtta

SEQ ID NO: 372 - DNA, 24 bp, synthetic construct
  source 1..24: mol_type = other DNA
agaataacagacttggaccatcag

SEQ ID NO: 373 - DNA, 24 bp, synthetic construct
  source 1..24: mol_type = other DNA
agatctgatggtccatctctgtta

SEQ ID NO: 374 - DNA, 24 bp, synthetic construct
  source 1..24: mol_type = other DNA
agaataacagagatggaccatcag

SEQ ID NO: 375 - DNA, 24 bp, synthetic construct
  source 1..24: mol_type = other DNA
agatctgatggtccatgactgtta

SEQ ID NO: 376 - DNA, 24 bp, synthetic construct
  source 1..24: mol_type = other DNA
agaataacagtcatggaccatcag

SEQ ID NO: 377 - DNA, 24 bp, synthetic construct
  source 1..24: mol_type = other DNA
agatctgatggtccatgtgtgtta

SEQ ID NO: 378 - DNA, 24 bp, synthetic construct
  source 1..24: mol_type = other DNA
agaataacacacatggaccatcag

SEQ ID NO: 379 - DNA, 24 bp, synthetic construct
  source 1..24: mol_type = other DNA
agatctgatggtccatgtcagtta

SEQ ID NO: 380 - DNA, 24 bp, synthetic construct
  source 1..24: mol_type = other DNA
agaataactgacatggaccatcag

SEQ ID NO: 381 - DNA, 24 bp, synthetic construct
  source 1..24: mol_type = other DNA
agatctgatggtccatgtctctta

SEQ ID NO: 382 - DNA, 24 bp, synthetic construct
  source 1..24: mol_type = other DNA
agaataagagacatggaccatcag

SEQ ID NO: 383 - DNA, 24 bp, synthetic construct
  source 1..24: mol_type = other DNA
agatctgatggtccatgtctgata

SEQ ID NO: 384 - DNA, 24 bp, synthetic construct
  source 1..24: mol_type = other DNA
agaatatcagacatggaccatcag

SEQ ID NO: 385 - DNA, 24 bp, synthetic construct
  source 1..24: mol_type = other DNA
agatctgatggtccatgtctgtaa

SEQ ID NO: 386 - DNA, 24 bp, synthetic construct
  source 1..24: mol_type = other DNA
agaattacagacatggaccatcag

SEQ ID NO: 387 - DNA, 24 bp, synthetic construct
  source 1..24: mol_type = other DNA
agatctgatggtccatgtctgttt

SEQ ID NO: 388 - DNA, 24 bp, synthetic construct
  source 1..24: mol_type = other DNA
agaaaaacagacatggaccatcag

SEQ ID NO: 389 - DNA, 200 bp, synthetic construct
  source 1..200: mol_type = other DNA
ccaagcttgccaccatgtacctggccactagtggcgggagccccgccgccaagagagtgaaattggacgccagtgggagtggatacccttatgacgtgcccgactatgcctacccttatgatgtcccagactacgcttatccctacgacgtaccggattatgccgaattcggcagtggatgaggatccgcaggcctctgc

SEQ ID NO: 390 - DNA, 272 bp, synthetic construct
  source 1..272: mol_type = other DNA
ccaagcttgccaccatgtacctggccactagtggcgggagccccgccgccaagagagtgaaattggacggcggcagtcccgccgctaaaagggtgaagctggatggtggatctcccgccgccaagagagttaagttggatgccagtgggagtggatacccttatgacgtgcccgactatgcctacccttatgatgtcccagactacgcttatccctacgacgtaccggattatgccgaattcggcagtggatgaggatccgcaggcctctgc

SEQ ID NO: 391 - DNA, 380 bp, synthetic construct
  source 1..380: mol_type = other DNA
ccaagcttgccaccatgtacctggccactagtggcgggagccccgccgccaagagagtgaaattggacggcggcagtcccgccgctaaaagggtgaagctggatggtggatctcccgccgccaagagagttaagttggatggcggctcccctgcagccaaacgggtgaaactcgacggcgggtccccggcagccaaaagggtaaagctggatggtggctctcccgcagctaaacgggtgaagctcgatgccagtgggagtggatacccttatgacgtgcccgactatgcctacccttatgatgtcccagactacgcttatccctacgacgtaccggattatgccgaattcggcagtggatgaggatccgcaggcctctgc

SEQ ID NO: 392 - DNA, 217 bp, synthetic construct
  source 1..217: mol_type = other DNA
cccgccagcggccgcgccaccatggactacccttatgacgtgcccgactatgcctacccttatgatgtcccagactacgcttatccctacgacgtaccggattatgccatgggcgggagccccgccgccaagagagtgaaattggacggcggcagtaagcttgccaccatgtacctggccactagttacctggcctaaggatccgcaggcctctgct

SEQ ID NO: 393 - DNA, 289 bp, synthetic construct
  source 1..289: mol_type = other DNA
cccgccagcggccgcgccaccatggactacccttatgacgtgcccgactatgcctacccttatgatgtcccagactacgcttatccctacgacgtaccggattatgccatgggcgggagccccgccgccaagagagtgaaattggacggcggcagtcccgccgctaaaagggtgaagctggatggtggatctcccgccgccaagagagttaagttggatggcggctccaagcttgccaccatgtacctggccactagttacctggcctaaggatccgcaggcctctgct

SEQ ID NO: 394 - DNA, 403 bp, synthetic construct
  source 1..403: mol_type = other DNA
cccgccagcggccgcgccaccatggactacccttatgacgtgcccgactatgcctacccttatgatgtcccagactacgcttatccctacgacgtaccggattatgccatgggcgggagccccgccgccaagagagtgaaattggacggcggcagtcccgccgctaaaagggtgaagctggatggtggatctcccgccgccaagagagttaagttggatggcggctcccctgcagccaaacgggtgaaactcgacggcgggtccccggcagccaaaagggtaaagctggatggtggctctcccgcagctaaacgggtgaagctcgatgccagtgggagtggaaagcttgccaccatgtacctggccactagttacctggcctaaggatccgcaggcctctgct

SEQ ID NO: 395 - DNA, 225 bp, synthetic construct
  source 1..225: mol_type = other DNA
cccgccagcggccgcgccaccatggacaagcttgccaccatgtacctggccactagtggcgggagccccgccgccaagagagtgaaattggacgccagtgggagtggatacccttatgacgtgcccgactatgcctacccttatgatgtcccagactacgcttatccctacgacgtaccggattatgccgaattcggcagtggatgaggatccgcaggcctctgc

SEQ ID NO: 396 - DNA, 238 bp, synthetic construct
  source 1..238: mol_type = other DNA
cccgccagcggccgcgccaccatggactacccttatgacgtgcccgactatgcctacccttatgatgtcccagactacgcttatccctacgacgtaccggattatgccatgggcgggagcaaaaggccggcggccacgaaaaaggccggccaggcaaaaaagaaaaagggcggcagtaagcttgccaccatgtacctggccactagttacctggcctaaggatccgcaggcctctgct

SEQ ID NO: 397 - DNA, 273 bp, synthetic construct
  source 1..273: mol_type = other DNA
cccgccagcggccgcgccaccatggacatgggcgggagccccgccgccaagagagtgaaattggacggcggcagtaagcttgccaccatgtacctggccactagtggcgggagccccgccgctaaaagggtgaagctggatgccagtgggagtggatacccttatgacgtgcccgactatgcctacccttatgatgtcccagactacgcttatccctacgacgtaccggattatgccgaattcggcagtggatgaggatccgcaggcctctgc

SEQ ID NO: 398 - DNA, 315 bp, synthetic construct
  source 1..315: mol_type = other DNA
cccgccagcggccgcgccaccatggacatgggcgggagcaaaaggccggcggccacgaaaaaggccggccaggcaaaaaagaaaaagggcggcagtaagcttgccaccatgtacctggccactagtggcgggagcaaaaggccggcggccacgaaaaaggccggccaggcaaaaaagaaaaaggccagtgggagtggatacccttatgacgtgcccgactatgcctacccttatgatgtcccagactacgcttatccctacgacgtaccggattatgccgaattcggcagtggatgaggatccgcaggcctctgc

SEQ ID NO: 399 - DNA, 54 bp, synthetic construct
  source 1..54: mol_type = other DNA
acactctttccctacacgacgctcttccgatctacgttcccttagcactctgcc

SEQ ID NO: 400 - DNA, 52 bp, synthetic construct
  source 1..52: mol_type = other DNA
gactggagttcagacgtgtgctcttccgatctgggagggcagaactagtcct

SEQ ID NO: 401 - RNA, 39 nt, synthetic construct
  source 1..39: mol_type = other RNA
aatttctactgtgtgtagactgatggtccatgtctgtta

SEQ ID NO: 402 - RNA, 39 nt, synthetic construct
  source 1..39: mol_type = other RNA
aatttctactgtgtgtagactgatggaccatgtctgtta

SEQ ID NO: 403 - RNA, 42 nt, synthetic construct
  source 1..42: mol_type = other RNA
aatttctactgtgtgtagattcaatctgtagcctctgggtct

SEQ ID NO: 404 - RNA, 41 nt, synthetic construct
  source 1..41: mol_type = other RNA
aatttctactgtgtgtagatcctcgctggactggtatttgt

SEQ ID NO: 405 - RNA, 4409 nt, synthetic construct
  source 1..4409: mol_type = other RNA
gacatttgcttctgacacaactgtgttcactagcaacctcaaacagacaccaagcttgccaccatgaacttccagcccttcttccagaagttcgtgcacctgtaccccatcagcaagaccctgagattcgagctgatcccccagggcgccacccagaagttcatcagcgagaagcaggtgctgctgcaggacgagatcagagccagaaagtaccccgagatgaagcaggccatcgacggctaccacaaggacttcatccagagagccctgagcaacatcgacagccaggtgttcgagcaggccctgaacaccttcgaggacctgttcctgagaagccaggccgagagagccaccgacgcctacaagaaggacttcgagaccgcccagaccaagctgagagagctgatcgtgcacagcttcgagaagggcgagttcaagcaggagtacaagagcctgttcgacaagaacctgatcaccaacctgctgaagccctgggtggagcagcagaaccagatcggcgacagcaactacacctaccacgaggacttcaacaagttcaccacctacttcctgggcttccacagaaacagaaagaacatctacagcaaggacccccacaagaccgccctggcctacagactgatccacgagaacctgcccaagttcctggagaacaacaagatcctgctgaagatccagaacgaccaccccagcctgtgggagcagctgcagaccctgaaccagaccatgccccagctgttcgacggctgggacttcagccagctgatgcaggtgagcttcttcagcaacaccctgacccagaccggcatcgaccagtacaacaccatcatcggcggcatcagcgagggcgagaacagacagaagatccagggcatcaacgagctgatcaacctgtacaaccagaagcaggacaagaagaacagagtggccaagctgaagcagctgtacaagcagatcctgagcgacagaagcaccctgagcttcctgcccgagaagttcgtggacgacaccgagctgtaccacgccatcaacatgttctacctggagcacctgcaccaccagagcatgatcaacggccacagctacaccctgctggagagagtgcagctgctgatcaacgagctggccaactacgacctgagcaaggtgtacctggcccccaaccagctgagcaccgtgagccaccagatgttcggcgacttcggctacatcggcagagccctgaactactactacatgcaggtgatccagcccgactacgagcagctgctggccagcgccaagaccaccaagaagatcgaggccaccgagaagctgaaaaccatcttcctggacaccccccagagcctggtggtgatccaggccgccatcgacgagtacatccagctgcagcccagcaccaagccccacacccagctgaccgacttcatcatcagcctgctgaagcagtacgagaccgtggccgacgaccagagcatcaaggtgatcaacgtgttcagcgacatcgagggcaagtacagctgcatcaagggcctggtgaacaccaagagcgagagcaagagagaggtgctgcaggacgagaagctggccaccgacatcaaggccttcatggacgccgtgaacaacgtgatcaagctgctgaagcccttcagcctgaacgagaagctggtggccagcgtggagaaggacgccagattctacagcgacttcgaggagatctaccagagcctgctgatcttcgtgcccctgtacaacaaggtgagaaactacatcacccagaagccctacagcaccgagaagttcaagctgaacttcaacaagcccaccctgctgagaggctgggacgccaacagagaggccgacaacctgagcatcctgctgagaaagaacggcaactactacctggccatcatggacaccgccaagggcgccaacaaggccttcgagcccaagaccctgaaccagctgaaggtggacgacaccaccgactgctacgagaagatggtgtacaagctgctgagcggccccagcaagatgttccccaaggccttcaaggccaagaacaacgagggcaactactaccccacccccgagctgctgaccagctacaacaacaacgagcacctgaagaacgacaagaacttcaccctggccagcctgcacgcctacatcgactggtgcaaggagtacatcaacagaaaccccagctggcaccagttcaacttcaagttcagccccacccagagcttccaggacatcagccagttctacagcgaggtgagcagccagagctacaaggtgcacttccagaccatccccagcgactacatcgaccagctggtggccgagggcaagctgtacctgttccagatctacaacaaggacttcagccccaacgccaagggcaaggagaacctgcacaccctgtacttcaaggccctgttcagcgacgagaacctgaagcagcccgtgttcaagctgagcggcgaggccgagatgttctacagacccgccagcctgcagctggccaacaccaccatccacaaggccggcgagcccatggccgccaagaaccccctgacccccaacgccaccagaaccctggcctacgacatcatcaaggacagaagattcaccaccgacaagtacctgctgcacgtgcccatcagcctgaacttccacgcccaggagagcatgagcatcaagaagcacaacgacctggtgagacagatgatcaagcacaaccaccaggacctgcacgtgatcggcatcgacagaggcgagaagcacctgctgtacgtgagcgtgatcgacctgaagggcaacatcgtgtaccaggagagcctgaacagcatcaagagcgaggcccagaacttcgagaccccctaccaccagctgctgcagcacagagaggagggcagagcccaggccagaaccgcctggggcaagatcgagaacatcaaggagctgaaggacggctacctgagccaggtggtgcacagaatccagcagctgatcctgaagtacaacgccatcgtgatgctggaggacctgaacttcggcttcaagagaggcagattcaagatcgagaagcagatctaccagaagttcgagaaggccctgatccacaagctgaactacgtggtggacaagagcacccaggccgacgagctgggcggcgtgagaaaggcctaccagctgaccgcccccttcgagagcttcgagaagctgggcaagcagagcggcgtgctgttctacgtgcccgcctggaacaccagcaagatcgaccccgtgaccggcttcgtggacctgctgaagcccaagtacgagaacctggacaaggcccaggccttcttcaacgccttcgacagcatccactacaacgcccagaagaactacttcgagttcaaggtgaacctgaagcagttcgccggcctgaaggcccaggccgcccaggccgagtggaccatctgcagctacggcgacgagagacacgtgtaccagaagaagaacgcccagcagggcgagaccgtgatcgtgaacgtgaccgaggagctgaaggtgctgttcgccaagaacaacatcgaggtggcccagagcgtggagctgaaggagaccatctgcacccagacccaggtggacttcttcaagagactgatgtggctgctgcaggtgctgctggccctgagatacagcagcagcaaggacaagctggactacatcctgagccccgtggccaacgcccagggcgagttcttcgacagcagacacgccagcgtgcagctgccccaggacagcgacgccaacggcgcctaccacatcgccctgaagggcctgtgggtgatcgagcagctgaaggccgccgacaacctggacaaggtgaagctggccatcagcaacgacgactggctgcacttcgcccagcagaagccctacctggccggcagaagcagcgacgacgaagcaacagccgatagccagcatgccgctccacctaagaagaagcggaaagtcggaggctctggcggcagcggtggaagcggcggttctggtggtagcggaggcagcggaggtagtggtggatctctggaaggcagttacccatacgatgttccagattacgcttatccctacgacgtgcctgattatgcatacccatatgatgtccccgactatgcctaagctgccttctgcggggcttgccttctggccatgcccttcttctctcccttgcacctgtacctcttggtctttgatttttttttttttttttttttttttttttttttttttttttttttttttttttttttttttttttttttttttttttttttttttttttttttttttttttttttttttttttttttttttttttttttttt

SEQ ID NO: 406 - RNA, 20 nt, synthetic construct
  source 1..20: mol_type = other RNA
aatttctactgtgtgtagat

SEQ ID NO: 407 - RNA, 18 nt, synthetic construct
  source 1..18: mol_type = other RNA
aatttctatgtgttagat

SEQ ID NO: 408 - RNA, 16 nt, synthetic construct
  source 1..16: mol_type = other RNA
aatttctatgttagat

SEQ ID NO: 409 - RNA, 19 nt, synthetic construct
  source 1..19: mol_type = other RNA
aatttctactgttgtagat

SEQ ID NO: 410 - RNA, 43 nt, synthetic construct
  source 1..43: mol_type = other RNA
aatttctactgtgtgtagatctgatggtccatgtctgttactc

SEQ ID NO: 411 - RNA, 40 nt, synthetic construct
  source 1..40: mol_type = other RNA
aatttctactgtgtgtagatctgatggtccatgtctgtta

SEQ ID NO: 412 - RNA, 38 nt, synthetic construct
  source 1..38: mol_type = other RNA
aatttctactgtgtgtagatctgatggtccatgtctgt

SEQ ID NO: 413 - RNA, 37 nt, synthetic construct
  source 1..37: mol_type = other RNA
aatttctactgtgtgtagatctgatggtccatgtctg

SEQ ID NO: 414 - RNA, 36 nt, synthetic construct
  source 1..36: mol_type = other RNA
aatttctactgtgtgtagatctgatggtccatgtct

SEQ ID NO: 415 - RNA, 35 nt, synthetic construct
  source 1..35: mol_type = other RNA
aatttctactgtgtgtagatctgatggtccatgtc

SEQ ID NO: 416 - DNA, 46 bp, synthetic construct
  source 1..46: mol_type = other DNA
tttgggcggggtccagttccgggattagcgaacttccaggccctcg

SEQ ID NO: 417 - protein, 79 aa, synthetic construct
GRSSDDEATADSQHAAPPKKKRKVGGSGGSGGSGGSGGSGGSGGSGGSLEGSYPYDVPDYAYPYDVPDYAYPYDVPDYA

SEQ ID NO: 418 - DNA, 36 bp, synthetic construct
  source 1..36: mol_type = other DNA
gtttaataagaatacataatttctactatcgtagat

SEQ ID NO: 419 - protein, 20 aa, unidentified
NKVRNYITQKPYSTEKFKLN

SEQ ID NO: 420 - protein, 24 aa, unidentified
DLHVIGIDRGEKHLLYVSVIDLKG

SEQ ID NO: 421 - protein, 40 aa, unidentified
KQSGVLFYVPAWNTSKIDPVTGFVDLLKPKYENLDKAQAF

SEQ ID NO: 422 - protein, 19 aa, unidentified
NMVRNYLTKKDYIDEKIRL

SEQ ID NO: 423 - protein, 22 aa, unidentified
DIYAIGIDRGERNLLYACVVNS

SEQ ID NO: 424 - protein, 38 aa, unidentified
KNQNGIVFYIPAWMTSKIDPVTGFTNLFYIKYESIEKS

SEQ ID NO: 425 - protein, 18 aa, unidentified
NMVRNYLTKKDYIEEKIR

SEQ ID NO: 426 - protein, 21 aa, unidentified
DIYAIGIDRGERNLLYACVVN

SEQ ID NO: 427 - protein, 38 aa, unidentified
KNQNGIVFYIPAWMTSKIDPVTGFTNLFYIKYESIEKA

SEQ ID NO: 428 - protein, 19 aa, unidentified
NLVRNFVTKKQIQTDKFKL

SEQ ID NO: 429 - protein, 24 aa, unidentified
DIKIIGIDRGEKHLAYYSVIDKTG

SEQ ID NO: 430 - protein, 40 aa, unidentified
ANYKQSGIMFYTRANYTSTTCPCCGFRKNIYISNSDTKEK

SEQ ID NO: 431 - protein, 19 aa, unidentified
NAVRNYLTKKNYSTDKIKL

SEQ ID NO: 432 - protein, 24 aa, unidentified
KISIIGIDRGEKNLIYFSMINENL

SEQ ID NO: 433 - protein, 39 aa, unidentified
YSQTGIIFYTQAAYTSVTCPNCSFRKNIYQKYENESKFK

SEQ ID NO: 434 - protein, 19 aa, unidentified
NKTRNYLTKKTYSLEKFKL

SEQ ID NO: 435 - protein, 24 aa, unidentified
DINIIGIDRGERHLLYACVINKNG

SEQ ID NO: 436 - protein, 41 aa, unidentified
KQNGVIFYIPAWCTSKIDPTTGFVNLFDLKKIDKEFVKKFN

SEQ ID NO: 437 - protein, 18 aa, unidentified
KVRNYVTQKPFSTDKIKL

SEQ ID NO: 438 - protein, 23 aa, unidentified
NNFIIGIDRGERNLLYVSVIDSN

SEQ ID NO: 439 - protein, 42 aa, unidentified
MQSGFIFYVRPDFTSKIDPVTGFVNLLYPRYENIDKAKDMIS

SEQ ID NO: 440 - protein, 18 aa, unidentified
NKTRNYITQKISDSTKKF

SEQ ID NO: 441 - protein, 23 aa, unidentified
VNIIGIDRGERNLIYMTLINQKG

SEQ ID NO: 442 - protein, 40 aa, unidentified
KQNGFLFYVPAAYTSKIDPTTGFVNLFNFRDLTNAEKKKD

SEQ ID NO: 443 - protein, 18 aa, unidentified
MVRNYVTRKPYSTDKFKL

SEQ ID NO: 444 - protein, 23 aa, unidentified
DVHVIGIDRGERNLLYLVVTDMD

SEQ ID NO: 445 - protein, 39 aa, unidentified
KQSGFLFYVPAWNTSKIDPATGFVNMLDLGYESIDKAKA

SEQ ID NO: 446 - protein, 20 aa, unidentified
KVRNYITTSRKPYKLDKYKL

SEQ ID NO: 447 - protein, 23 aa, unidentified
DNYIIGIDRGERNLLYVCVINSA

SEQ ID NO: 448 - protein, 40 aa, unidentified
SYQNGFIFYVPAWLTSKIDPTTGFVDLLKPKYTNVEEAHL

SEQ ID NO: 449 - protein, 19 aa, unidentified
NEFRNYLTKKPYAEDKIKL

SEQ ID NO: 450 - protein, 27 aa, unidentified
ARIIGIDRGEKHLLYISVIDENANILK

SEQ ID NO: 451 - protein, 42 aa, unidentified
KKQVGIIFYIPASFTSAICPVCGFRKRLYGFTFKNINQVKKL

SEQ ID NO: 452 - protein, 19 aa, unidentified
KTRNYLTQKPYSNEKIKIN

SEQ ID NO: 453 - protein, 23 aa, unidentified
DIHVIGIDRGERNLLYVCVIDKD

SEQ ID NO: 454 - protein, 40 aa, unidentified
GKQSGILYYIPAWCTSKIDPTTGFINRFYIKYENLEKSRE

SEQ ID NO: 455 - protein, 20 aa, unidentified
NKTRNYLTQKPYSTEKIKLN

SEQ ID NO: 456 - protein, 23 aa, unidentified
DINVIGIDRGERNLLYVCVIDKN

SEQ ID NO: 457 - protein, 39 aa, unidentified
KQSGVLYYIPAWCTSKIDPTTGFINRFYIKYENLDKSKE

SEQ ID NO: 458 - protein, 20 aa, unidentified
NKTRNYLTQKPYSIEKIKIN

SEQ ID NO: 459 - protein, 23 aa, unidentified
HFNIIGIDRGERNLLYITVINEK

SEQ ID NO: 460 - protein, 40 aa, unidentified
GKQSGILFYIPAWCTSKIDPTTGFVNLFKIKYKNINDAKV

SEQ ID NO: 461 - protein, 19 aa, unidentified
KVRNYVTKKPYSTEKIKLN

SEQ ID NO: 462 - protein, 23 aa, unidentified
NLYVIGVDRGERNLLYVSVVDAD

SEQ ID NO: 463 - protein, 37 aa, unidentified
FGQSGFVFYVPAWNTSNIDPCTGFTNLFFSKDLRYAS

SEQ ID NO: 464 - protein, 18 aa, unidentified
LIRNYLTKKSYSLDKWKL

SEQ ID NO: 465 - protein, 23 aa, unidentified
VNAIIGIDRGEKHLAYVAIVDRS

SEQ ID NO: 466 - protein, 38 aa, unidentified
YNHMGFVFFVDPSYTSAVDPKTGYRQHFRLDDRINTKT

SEQ ID NO: 467 - protein, 19 aa, unidentified
KVRSFASKKPYSTKKIKLN

SEQ ID NO: 468 - protein, 23 aa, unidentified
DVNIIGIDRGERNLLYISMIDRS

SEQ ID NO: 469 - protein, 39 aa, unidentified
KQTGFLFYVPAWNTSKIDPVTGFVDMLKPKYKSIPDARV

SEQ ID NO: 470 - protein, 19 aa, unidentified
KVRNFMTKKPYSLEKFKLN

SEQ ID NO: 471 - protein, 24 aa, unidentified
NIKVIGIDRGERHLLYLSLIDAEG

SEQ ID NO: 472 - protein, 40 aa, unidentified
GKQNGFLFYVPAWNTSKIDPVTGFVNLFDTRYASVEKSKA

SEQ ID NO: 473 - protein, 20 aa, unidentified
DKVRNYITQKPYSKDKIKLN

SEQ ID NO: 474 - protein, 23 aa, unidentified
DNYVIGIDRGERNLIYISVIDSN

SEQ ID NO: 475 - protein, 40 aa, unidentified
KQNGIIFYVPAWDTSKIDPVTGFVNLLKPKYTSVSEAKKL

SEQ ID NO: 476 - protein, 20 aa, unidentified
NKVRNWLTRKPYSTEKIKLN

SEQ ID NO: 477 - protein, 23 aa, unidentified
IKHIIGIDRGERHLLYISLIDLK

SEQ ID NO: 478 - protein, 40 aa, unidentified
KQSGCLFYIPAWNTSKIDPVTGFVNLFDTRYTNADQARKF

SEQ ID NO: 479 - protein, 20 aa, unidentified
NKVRNWLTRKPYSTEKIKLN

SEQ ID NO: 480 - protein, 24 aa, unidentified
GIKHVIGIDRGERHLLYLSLIDLK

SEQ ID NO: 481 - protein, 40 aa, unidentified
KQSGCLFYVPAWNTSKIDPVTGFVNLFDTKNVKGEKVRAF

SEQ ID NO: 482 - protein, 20 aa, unidentified
NKVRNYVTQKPYSTEKIKLN

SEQ ID NO: 483 - protein, 23 aa, unidentified
DIHVIGIDRGERNLLYVCVIDKN

SEQ ID NO: 484 - protein, 41 aa, unidentified
KQSGILYYIPAWCTSKIDPTTGFINRFYLKYENFDKSKEFV

SEQ ID NO: 485 - protein, 20 aa, unidentified
NKVRNYLTKKPYSEDKIKLN

SEQ ID NO: 486 - protein, 23 aa, unidentified
DLHVIGIDRGERHLLYLVVIDSK

SEQ ID NO: 487 - protein, 40 aa, unidentified
KQSGFLFYIPAWNTSKIDPMTGFVNLFDVKYESVEKAKAF

SEQ ID NO: 488 - protein, 34 aa, Acidaminococcus sp.
KLKDQKTPIPDTLYQELYDYVNHRLSHDLSDEAR